(19) United States
(12) Patent Application Publication
(10) Pub. No.: US 2023/0165913 A1
(43) Pub. Date: Jun. 1, 2023

(54) MICROBIAL CONSORTIA

(71) Applicant: Federation Bio, Inc., South San Francisco, CA (US)

(72) Inventors: Lee Robert Swem, Montara, CA (US); Pawan Kumar, Belmont, CA (US); Aditya Bhalla, Union City, CA (US); Shital A. Tripathi, Brisbane, CA (US); Anupreet Parmar, Milpitas, CA (US); Joshua J. Hamilton, Montara, CA (US); Ariel R. Brumbaugh, San Francisco, CA (US); Dante P. Ricci, Richmond, CA (US); Hans Richard William Layman, Pacifica, CA (US); Ariana M. Ciglar, Pacifica, CA (US); James Berleman, Richmond, CA (US); Zachary Walters, Brisbane, CA (US); Kyle Jacoby, Burlingame, CA (US); Nicholas D. Youngblut, Foster City, CA (US); Andreas Grauer, San Francisco, CA (US); Emily Drabant Conley, Redwood City, CA (US); Heather Romasko, Burlingame, CA (US)

(73) Assignee: Federation Bio, Inc., South San Francisco, CA (US)

(21) Appl. No.: 18/060,831

(22) Filed: Dec. 1, 2022

Related U.S. Application Data
(60) Provisional application No. 63/305,476, filed on Feb. 1, 2022.
(60) Provisional application No. 63/285,010, filed on Dec. 1, 2021.

(51) Int. Cl. A61K 35/741; A61K 45/06; A61P 13/04; A61K 38/45; A61K 38/51; C12N 1/20

(52) U.S. Cl. CPC A61K 35/741; A61K 45/06; A61P 13/04; A61K 38/45; A61K 38/51; C12N 1/20

(57) ABSTRACT

The present disclosure provides microbial consortia comprising O. formigenes capable of stable engraftment in the gastrointestinal tract of a subject and methods of using and making the same.

### CROSS-REFERENCE TO RELATED APPLICATIONS

This application claims priority to U.S. Provisional Patent Application No. 63/285,010, filed on Dec. 1, 2021, and to U.S. Provisional Patent Application No. 63/305,476, filed on Feb. 8, 2022, the content of each of which is incorporated by reference in its entirety, and to each of which priority is claimed.

### SEQUENCE LISTING

The instant application contains a Sequence Listing which has been submitted electronically in XML format and is hereby incorporated by reference in its entirety. Said XML copy, created on Dec. 1, 2022, is named 091592.0107.xml and is 393,232 bytes in size.

### FIELD OF THE INVENTION

The present disclosure generally relates to microbial consortia for administration to an animal for degradation of a disease-associated metabolic substrate.

### BACKGROUND

The gastrointestinal tract comprises various biological niches along its longitudinal length having different physical, chemical, and nutrient compositions. As a consequence of these diverse conditions, specific microbial communities are established within a particular biological niche. The microbial species comprising a specific microbial community are highly responsive to their local environment and produce an array of bioactive molecules that facilitate host engraftment, inter-microbial communication, nutrient metabolism, and inclusion or exclusion of competing microbial species. Adding further complexity, there is substantial diversity of microbial species and strains in the human gastrointestinal tract between individuals, which is attributed to a number of factors including genetics, diet, antibiotic and antifungal use, surgical intervention (e.g., gastric by-pass/bowel resection), presence of inflammatory bowel disease and/or irritable bowel syndrome, and other environmental influences. However, despite this interindividual diversity, the functional attributes of the varying human gut microbiota are relatively consistent among healthy adults and comprise core metabolic pathways involved in carbohydrate metabolism, amino acid metabolism, fermentation, and oxidative phosphorylation.

Modulation of microbial species in the gastrointestinal tract through the use of antibiotics, antifungals, and more recently, fecal microbial transplantation (“FMT”), have been approaches clinically investigated for the treatment and/or prevention of certain diseases and disorders. For example, Dodd et al. (Nature, 2007, 551: 648-652) have proposed FMT as a therapeutic to modulate the levels of aromatic amino acid metabolites in the serum of gnotobiotic mice, which affect intestinal permeability and systemic immunity. In further examples, administration of bacterial compositions have also been proposed as a method for treating Clostridium difficile infection, ulcerative colitis, cholestatic disease, and hyperoxaluria.

As a modality for treating various diseases and/or conditions, there is a need for microbial compositions comprising a plurality of microbial species having improved therapeutic efficacy and an ability to efficiently engraft in a host, grow, and metabolize pathogenic substrates to non-pathogenic metabolic products within the various biological niches of the gastrointestinal tract and within the diverse gastrointestinal environments of different individuals. Furthermore, there is an unmet need for a treatment of diseases using a complex microbial community that can engraft and function symbiotically in the human gastrointestinal tract to degradation of a disease-associated metabolic substrate.

### SUMMARY OF THE INVENTION

The present disclosure relates to compositions and methods for reducing oxalate in a subject. In certain non-limiting embodiments, the present disclosure provides a composition comprising at least 1 oxalate-metabolizing microbial strain. In certain embodiments, the at least one strain expresses an enzyme selected from a formyl-CoA transferase, an oxalate-formate antiporter, and an oxalyl-CoA decarboxylase. In certain embodiments, the at least 1 oxalate-metabolizing microbial strain is from the Oxalobacter genus.

In certain embodiments, the composition comprises at least 3 oxalate-metabolizing microbial strains. In certain embodiments, the at least 3 oxalate-metabolizing microbial strains are different strains of the same species. In certain embodiments, the at least 3 oxalate-metabolizing microbial strains are different strains of different species.

In certain embodiments, the species is Oxalobacter formigenes (O. formigenes), and optionally wherein the number of oxalate-metabolizing microbial strains is 3 or more.

In certain embodiments:

a) at least one strain is a low pH tolerance strain;

b) at least one strain is a high oxalate tolerance strain; and/or

c) at least one strain is a high growth rate strain.

In certain non-limiting embodiments, the present disclosure provides a composition comprising at least 2 Oxalobacter formigenes (O. formigenes) strains, wherein each of the strains comprises one or more of the following functions: a) a low pH tolerance strain; b) a high oxalate tolerance strain; and/or c) a high growth rate strain.

The present disclosure further provides a composition comprising at least 3 Oxalobacter formigenes (O. formigenes) strains, wherein: a) at least one strain is a low pH tolerance strain; b) at least one strain is a high oxalate tolerance strain; and c) at least one strain is a high growth rate strain.

In certain embodiments, the low pH tolerance strain can metabolize oxalate at a pH between about 4 and about 6. In certain embodiments, the low pH tolerance strain can metabolize oxalate at a pH of about 5. In certain embodiments, the high oxalate tolerance strain can metabolize oxalate at a concentration between about 5 mM to about 30 mM. In certain embodiments, the high oxalate tolerance strain can metabolize oxalate at a concentration of about 15 mM.

In certain embodiments, each strain comprises a 16s RNA nucleotide sequence that is (a) at least about 80% identical to the nucleotide sequence set forth in SEQ ID NO: 42, SEQ ID NO: 79, or SEQ ID NO: 146, (b) at least about 90% identical to the nucleotide sequence set forth in SEQ ID NO: 42, SEQ ID NO: 79, or SEQ ID NO: 146, or (c) at least about 96% identical to the nucleotide sequence set forth in SEQ ID NO: 42, SEQ ID NO: 79, or SEQ ID NO: 146. In certain embodiments, each strain comprises a 16s RNA nucleotide sequence that is at least about 97% identical or 98.5% identical to the nucleotide sequence set forth in SEQ ID NO: 42, SEQ ID NO: 79, or SEQ ID NO: 146. In certain embodiments, each strain comprises a 16s RNA nucleotide sequence identical to the nucleotide sequence set forth in SEQ ID NO: 42, SEQ ID NO: 79, or SEQ ID NO: 146.

In certain embodiments, the composition further comprises one or more microbes metabolizing formate. In certain embodiments, the composition further comprises one or more microbes catalyzing fermentation of polysaccharides. In certain embodiments, the composition further comprises one or more microbes catalyzing fermentation of amino acids. In certain embodiments, the composition further comprises microbes catalyzing the synthesis of at least one molecules selected from the group consisting of methane, acetate, sulfide, propionate, and succinate. In certain embodiments, the composition further comprises microbes catalyzing deconjugation of conjugated bile acids to produce primary bile acids. In certain embodiments, the composition further comprises microbes catalyzing conversion of cholic acid (CA) to 7-oxocholic acid. In certain embodiments, the composition further comprises microbes catalyzing conversion of 7-oxocholic acid to 7-beta-cholic acid (7betaCA). In certain embodiments, the composition further comprises microbes catalyzing conversion of chenodeoxycholic acid (CDCA) to 7-oxochenodeoxycholic acid. In certain embodiments, the composition further comprises microbes catalyzing conversion of 7-oxochenodeoxycholic acid to ursodeoxycholic acid (UDCA).

In certain embodiments, the composition comprises: a) Consortia I or a functional equivalent thereof, b) Consortia II or a functional equivalent thereof; c) Consortia III or a functional equivalent thereof, d) Consortia IV or a functional equivalent thereof, e) Consortia V or a functional equivalent thereof, f) Consortia VI or a functional equivalent thereof, g) Consortia VII or a functional equivalent thereof, h) Consortia VIII or a functional equivalent thereof; i) Consortia IX or a functional equivalent thereof, j) Consortia X or a functional equivalent thereof, k) Consortia XI or a functional equivalent thereof, l) Consortia XII or a functional equivalent thereof, m) Consortia XIII or a functional equivalent thereof, n) Consortia XIV or a functional equivalent thereof, o) Consortia XV or a functional equivalent thereof, p) Consortia XVI or a functional equivalent thereof, q) Consortia XVII or a functional equivalent thereof, r) Consortia XVIII or a functional equivalent thereof, or s) Consortia XIX or a functional equivalent thereof.

In certain embodiments, the composition further comprises a second composition comprising Clostridium citroniae, Bacteroides salyersiae, Blautia obeum, Parabacteroides merdae, Parabacteroides distasonis, Anaerostipes hadrus, Lachnospiraceae sp. FBI00033, Eubacterium eligens, Bifidobacterium dentium, Blautia wexlerae, Fusicatenibacter saccharivorans, Bacteroides nordii, Dorea formicigenerans, Dorea longicatena, Bacteroides stercorirosoris, Bifidobacterium longum, Bacteroides kribbi, Lachnospiraceae sp. FBI00071, Bacteroides thetaiotaomicron, Clostridium clostridioforme, Clostridium scindens, Roseburia hominis, Clostridium fessum, Coprococcus comes, Blautia faecis, Hungatella hathewayi, Bacteroides stercoris, Collinsella aerofaciens, Hungatella effluvii, Bifidobacterium adolescentis, Bifidobacterium catenulatum, Lactobacillus rogosae, Bacteroides faecis, Bacteroides finegoldii, Clostridiaceae sp. FBI00191, Ruminococcus faecis, Lachnoclostridium pacaense, Clostridium bolteae, Longicatena caecimuris, Eggerthella lenta, Blautia massiliensis, Bacteroides xylanisolvens, Bacteroides vulgatus, Megasphaera massiliensis, Butyricimonas faecihominis, Eisenbergiella tayi, Acidaminococcus intestini, Emergencia timonensis, Bifidobacterium pseudocatenulatum, Eubacterium hallii, Anaerofustis stercorihominis, Eubacterium ventriosum, Blautia hydrogenotrophica, Lachnospiraceae sp. FBI00290, or a functional equivalent microbial consortium.

In certain embodiments, the composition further comprises FBI00001, FBI00002, FBI00010, FBI00013, FBI00029, FBI00032, FBI00033, FBI00034, FBI00043, FBI00044, FBI00048, FBI00050, FBI00051, FBI00057, FBI00059, FBI00060, FBI00070, FBI00071, FBI00076, FBI00079, FBI00087, FBI00093, FBI00102, FBI00109, FBI00117, FBI00120, FBI00125, FBI00127, FBI00128, FBI00145, FBI00162, FBI00174, FBI00184, FBI00190, FBI00191, FBI00194, FBI00198, FBI00199, FBI00200, FBI00201, FBI00205, FBI00206, FBI00211, FBI00220, FBI00221, FBI00236, FBI00245, FBI00248, FBI00251, FBI00254, FBI00267, FBI00278, FBI00288, FBI00290, or a functional equivalent thereof.

In certain embodiments, each strain comprises a 16s RNA nucleotide sequence that is (a) at least about 80% identical to the nucleotide sequence set forth in SEQ ID NO: 1, SEQ ID NO: 2, SEQ ID NO: 5, SEQ ID NO: 8, SEQ ID NO: 18, SEQ ID NO: 20, SEQ ID NO: 21, SEQ ID NO: 22, SEQ ID NO: 25, SEQ ID NO: 26, SEQ ID NO: 29, SEQ ID NO: 31, SEQ ID NO: 32, SEQ ID NO: 36, SEQ ID NO: 37, SEQ ID NO: 38, SEQ ID NO: 45, SEQ ID NO: 46, SEQ ID NO: 48, SEQ ID NO: 51, SEQ ID NO: 55, SEQ ID NO: 57, SEQ ID NO: 61, SEQ ID NO: 63, SEQ ID NO: 70, SEQ ID NO: 71, SEQ ID NO: 74, SEQ ID NO: 76, SEQ ID NO: 77, SEQ ID NO: 83, SEQ ID NO: 89, SEQ ID NO: 94, SEQ ID NO: 100, SEQ ID NO: 102, SEQ ID NO: 103, SEQ ID NO: 104, SEQ ID NO: 106, SEQ ID NO: 107, SEQ ID NO: 108, SEQ ID NO: 109, SEQ ID NO: 110, SEQ ID NO: 111, SEQ ID NO: 113, SEQ ID NO: 115, SEQ ID NO: 116, SEQ ID NO: 123, SEQ ID NO: 128, SEQ ID NO: 129, SEQ ID NO: 130, SEQ ID NO: 131, SEQ ID NO: 136, SEQ ID NO: 143, SEQ ID NO: 145, or SEQ ID NO: 147, (b) at least about 90% identical to the nucleotide sequence set forth in SEQ ID NO: 1, SEQ ID NO: 2, SEQ ID NO: 5, SEQ ID NO: 8, SEQ ID NO: 18, SEQ ID NO: 20, SEQ ID NO: 21, SEQ ID NO: 22, SEQ ID NO: 25, SEQ ID NO: 26, SEQ ID NO: 29, SEQ ID NO: 31, SEQ ID NO: 32, SEQ ID NO: 36, SEQ ID NO: 37, SEQ ID NO: 38, SEQ ID NO: 45, SEQ ID NO: 46, SEQ ID NO: 48, SEQ ID NO: 51, SEQ ID NO: 55, SEQ ID NO: 57, SEQ ID NO: 61, SEQ ID NO: 63, SEQ ID NO: 70, SEQ ID NO: 71, SEQ ID NO: 74, SEQ ID NO: 76, SEQ ID NO: 77, SEQ ID NO: 83, SEQ ID NO: 89, SEQ ID NO: 94, SEQ ID NO: 100, SEQ ID NO: 102, SEQ ID NO: 103, SEQ ID NO: 104, SEQ ID NO: 106, SEQ ID NO: 107, SEQ ID NO: 108, SEQ ID NO: 109, SEQ ID NO: 110, SEQ ID NO: 111, SEQ ID NO: 113, SEQ ID NO: 115, SEQ ID NO: 116, SEQ ID NO: 123, SEQ ID NO: 128, SEQ ID NO: 129, SEQ ID NO: 130, SEQ ID NO: 131, SEQ ID NO: 136, SEQ ID NO: 143, SEQ ID NO: 145, or SEQ ID NO: 147, or (c) at least about 96% identical to the nucleotide sequence set forth in SEQ ID NO: 1, SEQ ID NO: 2, SEQ ID NO: 5, SEQ ID NO: 8, SEQ ID NO: 18, SEQ ID NO: 20, SEQ ID NO: 21, SEQ ID NO: 22, SEQ ID NO: 25, SEQ ID NO: 26, SEQ ID NO: 29, SEQ ID NO: 31, SEQ ID NO: 32, SEQ ID NO: 36, SEQ ID NO: 37, SEQ ID NO: 38, SEQ ID NO: 45, SEQ ID NO: 46, SEQ ID NO: 48, SEQ ID NO: 51, SEQ ID NO: 55, SEQ ID NO: 57, SEQ ID NO: 61, SEQ ID NO: 63, SEQ ID NO: 70, SEQ ID NO: 71, SEQ ID NO: 74, SEQ ID NO: 76, SEQ ID NO: 77, SEQ ID NO: 83, SEQ ID NO: 89, SEQ ID NO: 94, SEQ ID NO: 100, SEQ ID NO: 102, SEQ ID NO: 103, SEQ ID NO: 104, SEQ ID NO: 106, SEQ ID NO: 107, SEQ ID NO: 108, SEQ ID NO: 109, SEQ ID NO: 110, SEQ ID NO: 111, SEQ ID NO: 113, SEQ ID NO: 115, SEQ ID NO: 116, SEQ ID NO: 123, SEQ ID NO: 128, SEQ ID NO: 129, SEQ ID NO: 130, SEQ ID NO: 131, SEQ ID NO: 136, SEQ ID NO: 143, SEQ ID NO: 145, or SEQ ID NO: 147.

In certain embodiments, each strain comprises a 16s RNA nucleotide sequence that is at least about 97% identical or 98.5% identical to the nucleotide sequence set forth in SEQ ID NO: 1, SEQ ID NO: 2, SEQ ID NO: 5, SEQ ID NO: 8, SEQ ID NO: 18, SEQ ID NO: 20, SEQ ID NO: 21, SEQ ID NO: 22, SEQ ID NO: 25, SEQ ID NO: 26, SEQ ID NO: 29, SEQ ID NO: 31, SEQ ID NO: 32, SEQ ID NO: 36, SEQ ID NO: 37, SEQ ID NO: 38, SEQ ID NO: 45, SEQ ID NO: 46, SEQ ID NO: 48, SEQ ID NO: 51, SEQ ID NO: 55, SEQ ID NO: 57, SEQ ID NO: 61, SEQ ID NO: 63, SEQ ID NO: 70, SEQ ID NO: 71, SEQ ID NO: 74, SEQ ID NO: 76, SEQ ID NO: 77, SEQ ID NO: 83, SEQ ID NO: 89, SEQ ID NO: 94, SEQ ID NO: 100, SEQ ID NO: 102, SEQ ID NO: 103, SEQ ID NO: 104, SEQ ID NO: 106, SEQ ID NO: 107, SEQ ID NO: 108, SEQ ID NO: 109, SEQ ID NO: 110, SEQ ID NO: 111, SEQ ID NO: 113, SEQ ID NO: 115, SEQ ID NO: 116, SEQ ID NO: 123, SEQ ID NO: 128, SEQ ID NO: 129, SEQ ID NO: 130, SEQ ID NO: 131, SEQ ID NO: 136, SEQ ID NO: 143, SEQ ID NO: 145, or SEQ ID NO: 147.

In certain embodiments, each strain comprises a 16s RNA nucleotide sequence identical to the nucleotide sequence set forth in SEQ ID NO: SEQ ID NO: 1, SEQ ID NO: 2, SEQ ID NO: 5, SEQ ID NO: 8, SEQ ID NO: 18, SEQ ID NO: 20, SEQ ID NO: 21, SEQ ID NO: 22, SEQ ID NO: 25, SEQ ID NO: 26, SEQ ID NO: 29, SEQ ID NO: 31, SEQ ID NO: 32, SEQ ID NO: 36, SEQ ID NO: 37, SEQ ID NO: 38, SEQ ID NO: 45, SEQ ID NO: 46, SEQ ID NO: 48, SEQ ID NO: 51, SEQ ID NO: 55, SEQ ID NO: 57, SEQ ID NO: 61, SEQ ID NO: 63, SEQ ID NO: 70, SEQ ID NO: 71, SEQ ID NO: 74, SEQ ID NO: 76, SEQ ID NO: 77, SEQ ID NO: 83, SEQ ID NO: 89, SEQ ID NO: 94, SEQ ID NO: 100, SEQ ID NO: 102, SEQ ID NO: 103, SEQ ID NO: 104, SEQ ID NO: 106, SEQ ID NO: 107, SEQ ID NO: 108, SEQ ID NO: 109, SEQ ID NO: 110, SEQ ID NO: 111, SEQ ID NO: 113, SEQ ID NO: 115, SEQ ID NO: 116, SEQ ID NO: 123, SEQ ID NO: 128, SEQ ID NO: 129, SEQ ID NO: 130, SEQ ID NO: 131, SEQ ID NO: 136, SEQ ID NO: 143, SEQ ID NO: 145, or SEQ ID NO: 147.

In certain embodiments, the composition further comprises a third composition comprising Acutalibacter timonensis, Alistipes onderdonkii, Bacteroides uniformis, Eubacterium rectale, Alistipes timonensis, Bacteroides kribbi, Coprococcus eutactus, Bilophila wadsworthia, Bacteroides caccae, Alistipes shahii, Parasutterella excrementihominis, Paraprevotella clara, Sutterella wadsworthensis, Sutterella massiliensis, Porphyromonas asaccharolytica, Ruminococcus bromii, Monoglobus pectinilyticus, Ruminococcaceae sp. FBI00097, Gordonibacter pamelaeae, Bacteroides uniformis, Gordonibacter pamelaeae, Bacteroides fragilis, Phascolarctobacterium faecium, Monoglobus pectinilyticus, Clostridium aldenense, Ruthenibacterium lactatiformans, Bacteroides ovatus, Bifidobacterium bifidum, Anaerotruncus massiliensis, Clostridium aldenense, Sutterella wadsworthensis, Catabacter hongkongensis, Alistipes senegalensis, Ruminococcaceae sp. FBI00233, Alistipes shahii, Dielma fastidiosa, Eubacterium siraeum, Faecalibacterium prausnitzii, Turicibacter sanguinis, Eubacterium rectale, Bacteroides caccae, Methanobrevibacter smithii, Barnesiella intestinihominis, Alistipes onderdonkii, Methanobrevibacter smithii, or a functional equivalent thereof.

In certain embodiments, the composition further comprises FBI00004, FBI00012, FBI00015, FBI00018, FBI00019, FBI00021, FBI00038, FBI00040, FBI00046, FBI00061, FBI00066, FBI00075, FBI00077, FBI00080, FBI00081, FBI00085, FBI00092, FBI00097, FBI00099, FBI00112, FBI00132, FBI00137, FBI00140, FBI00149, FBI00151, FBI00176, FBI00189, FBI00197, FBI00208, FBI00212, FBI00224, FBI00226, FBI00229, FBI00233, FBI00235, FBI00237, FBI00243, FBI00244, FBI00258, FBI00260, FBI00263, FBI00270, FBI00273, FBI00277, FBI00292, or a functional equivalent thereof.

In certain embodiments, each strain comprises a 16s RNA nucleotide sequence that is (a) at least about 80% identical to the nucleotide sequence set forth in SEQ ID NO: 3, SEQ ID NO: 7, SEQ ID NO: 9, SEQ ID NO: 11, SEQ ID NO: 12, SEQ ID NO: 14, SEQ ID NO: 23, SEQ ID NO: 24, SEQ ID NO: 27, SEQ ID NO: 39, SEQ ID NO: 41, SEQ ID NO: 47, SEQ ID NO: 49, SEQ ID NO: 50, SEQ ID NO: 51, SEQ ID NO: 54, SEQ ID NO: 56, SEQ ID NO: 59, SEQ ID NO: 60, SEQ ID NO: 66, SEQ ID NO: 78, SEQ ID NO: 81, SEQ ID NO: 82, SEQ ID NO: 85, SEQ ID NO: 86, SEQ ID NO: 96, SEQ ID NO: 101, SEQ ID NO: 105, SEQ ID NO: 112, SEQ ID NO: 114, SEQ ID NO: 117, SEQ ID NO: 118, SEQ ID NO: 119, SEQ ID NO: 121, SEQ ID NO: 122, SEQ ID NO: 124, SEQ ID NO: 126, SEQ ID NO: 127, SEQ ID NO: 133, SEQ ID NO: 134, SEQ ID NO: 135, SEQ ID NO: 138, SEQ ID NO: 140, SEQ ID NO: 142, or SEQ ID NO: 148, (b) at least about 90% identical to the nucleotide sequence set forth in SEQ ID NO: 3, SEQ ID NO: 7, SEQ ID NO: 9, SEQ ID NO: 11, SEQ ID NO: 12, SEQ ID NO: 14, SEQ ID NO: 23, SEQ ID NO: 24, SEQ ID NO: 27, SEQ ID NO: 39, SEQ ID NO: 41, SEQ ID NO: 47, SEQ ID NO: 49, SEQ ID NO: 50, SEQ ID NO: 51, SEQ ID NO: 54, SEQ ID NO: 56, SEQ ID NO: 59, SEQ ID NO: 60, SEQ ID NO: 66, SEQ ID NO: 78, SEQ ID NO: 81, SEQ ID NO: 82, SEQ ID NO: 85, SEQ ID NO: 86, SEQ ID NO: 96, SEQ ID NO: 101, SEQ ID NO: 105, SEQ ID NO: 112, SEQ ID NO: 114, SEQ ID NO: 117, SEQ ID NO: 118, SEQ ID NO: 119, SEQ ID NO: 121, SEQ ID NO: 122, SEQ ID NO: 124, SEQ ID NO: 126, SEQ ID NO: 127, SEQ ID NO: 133, SEQ ID NO: 134, SEQ ID NO: 135, SEQ ID NO: 138, SEQ ID NO: 140, SEQ ID NO: 142, or SEQ ID NO: 148, or (c) at least about 96% identical to the nucleotide sequence set forth in SEQ ID NO: 3, SEQ ID NO: 7, SEQ ID NO: 9, SEQ ID NO: 11, SEQ ID NO: 12, SEQ ID NO: 14, SEQ ID NO: 23, SEQ ID NO: 24, SEQ ID NO: 27, SEQ ID NO: 39, SEQ ID NO: 41, SEQ ID NO: 47, SEQ ID NO: 49, SEQ ID NO: 50, SEQ ID NO: 51, SEQ ID NO: 54, SEQ ID NO: 56, SEQ ID NO: 59, SEQ ID NO: 60, SEQ ID NO: 66, SEQ ID NO: 78, SEQ ID NO: 81, SEQ ID NO: 82, SEQ ID NO: 85, SEQ ID NO: 86, SEQ ID NO: 96, SEQ ID NO: 101, SEQ ID NO: 105, SEQ ID NO: 112, SEQ ID NO: 114, SEQ ID NO: 117, SEQ ID NO: 118, SEQ ID NO: 119, SEQ ID NO: 121, SEQ ID NO: 122, SEQ ID NO: 124, SEQ ID NO: 126, SEQ ID NO: 127, SEQ ID NO: 133, SEQ ID NO: 134, SEQ ID NO: 135, SEQ ID NO: 138, SEQ ID NO: 140, SEQ ID NO: 142, or SEQ ID NO: 148.

In certain embodiments, each strain comprises a 16s RNA nucleotide sequence that is at least about 97% identical or 98.5% identical to the nucleotide sequence set forth in SEQ ID NO: 3, SEQ ID NO: 7, SEQ ID NO: 9, SEQ ID NO: 11, SEQ ID NO: 12, SEQ ID NO: 14, SEQ ID NO: 23, SEQ ID NO: 24, SEQ ID NO: 27, SEQ ID NO: 39, SEQ ID NO: 41, SEQ ID NO: 47, SEQ ID NO: 49, SEQ ID NO: 50, SEQ ID NO: 51, SEQ ID NO: 54, SEQ ID NO: 56, SEQ ID NO: 59, SEQ ID NO: 60, SEQ ID NO: 66, SEQ ID NO: 78, SEQ ID NO: 81, SEQ ID NO: 82, SEQ ID NO: 85, SEQ ID NO: 86, SEQ ID NO: 96, SEQ ID NO: 101, SEQ ID NO: 105, SEQ ID NO: 112, SEQ ID NO: 114, SEQ ID NO: 117, SEQ ID NO: 118, SEQ ID NO: 119, SEQ ID NO: 121, SEQ ID NO: 122, SEQ ID NO: 124, SEQ ID NO: 126, SEQ ID NO: 127, SEQ ID NO: 133, SEQ ID NO: 134, SEQ ID NO: 135, SEQ ID NO: 138, SEQ ID NO: 140, SEQ ID NO: 142, or SEQ ID NO: 148.

In certain embodiments, each strain comprises a 16s RNA nucleotide sequence identical to the nucleotide sequence set forth in SEQ ID NO: SEQ ID NO: 3, SEQ ID NO: 7, SEQ ID NO: 9, SEQ ID NO: 11, SEQ ID NO: 12, SEQ ID NO: 14, SEQ ID NO: 23, SEQ ID NO: 24, SEQ ID NO: 27, SEQ ID NO: 39, SEQ ID NO: 41, SEQ ID NO: 47, SEQ ID NO: 49, SEQ ID NO: 50, SEQ ID NO: 51, SEQ ID NO: 54, SEQ ID NO: 56, SEQ ID NO: 59, SEQ ID NO: 60, SEQ ID NO: 66, SEQ ID NO: 78, SEQ ID NO: 81, SEQ ID NO: 82, SEQ ID NO: 85, SEQ ID NO: 86, SEQ ID NO: 96, SEQ ID NO: 101, SEQ ID NO: 105, SEQ ID NO: 112, SEQ ID NO: 114, SEQ ID NO: 117, SEQ ID NO: 118, SEQ ID NO: 119, SEQ ID NO: 121, SEQ ID NO: 122, SEQ ID NO: 124, SEQ ID NO: 126, SEQ ID NO: 127, SEQ ID NO: 133, SEQ ID NO: 134, SEQ ID NO: 135, SEQ ID NO: 138, SEQ ID NO: 140, SEQ ID NO: 142, or SEQ ID NO: 148.

In certain embodiments, the composition further comprises a fourth composition comprising Bifidobacterium adolescentis, Bifidobacterium longum, Bifidobacterium pseudocatenulatum, Bacteroides thetaiotaomicron, Coprococcus comes, Fusicatenibacter saccharivorans, Eggerthella lenta, Eubacterium eligens, Bacteroides xylanisolvens, Lactobacillus rogosae, Clostridium citroniae, Collinsella aerofaciens, Blautia obeum, Eggerthella lenta, Blautia wexlerae, Lachnoclostridium pacaense, Bacteroides vulgatus, Parabacteroides merdae, Dorea formicigenerans, Ruminococcus faecis, Roseburia hominis, Anaerostipes hadrus, Bifidobacterium adolescentis, Bifidobacterium pseudocatenulatum, Clostridium bolteae, Eisenbergiella tayi, Dorea longicatena, Eggerthella lenta, Bacteroides stercoris, Hungatella hathewayi, Bacteroides xylanisolvens, or a functional equivalent thereof.

In certain embodiments, the composition further comprises FBI00009, FBI00011, FBI00016, FBI00020, FBI00025, FBI00027, FBI00030, FBI00047, FBI00052, FBI00053, FBI00056, FBI00062, FBI00078, FBI00096, FBI00104, FBI00110, FBI00111, FBI00113, FBI00115, FBI00116, FBI00123, FBI00124, FBI00126, FBI00135, FBI00147, FBI00159, FBI00167, FBI00170, FBI00232, FBI00255, FBI00271, or a functional equivalent thereof.

In certain embodiments, each strain comprises a 16s RNA nucleotide sequence that is (a) at least about 80% identical to the nucleotide sequence set forth in SEQ ID NO: 4, SEQ ID NO: 6, SEQ ID NO: 10, SEQ ID NO: 13, SEQ ID NO: 16, SEQ ID NO: 17, SEQ ID NO: 19, SEQ ID NO: 28, SEQ ID NO: 33, SEQ ID NO: 34, SEQ ID NO: 35, SEQ ID NO: 40, SEQ ID NO: 50, SEQ ID NO: 58, SEQ ID NO: 62, SEQ ID NO: 64, SEQ ID NO: 65, SEQ ID NO: 67, SEQ ID NO: 68, SEQ ID NO: 69, SEQ ID NO: 72, SEQ ID NO: 73, SEQ ID NO: 75, SEQ ID NO: 80, SEQ ID NO: 84, SEQ ID NO: 88, SEQ ID NO: 91, SEQ ID NO: 92, SEQ ID NO: 120, SEQ ID NO: 132, or SEQ ID NO: 139, (b) at least about 90% identical to the nucleotide sequence set forth in SEQ ID NO: 4, SEQ ID NO: 6, SEQ ID NO: 10, SEQ ID NO: 13, SEQ ID NO: 16, SEQ ID NO: 17, SEQ ID NO: 19, SEQ ID NO: 28, SEQ ID NO: 33, SEQ ID NO: 34, SEQ ID NO: 35, SEQ ID NO: 40, SEQ ID NO: 50, SEQ ID NO: 58, SEQ ID NO: 62, SEQ ID NO: 64, SEQ ID NO: 65, SEQ ID NO: 67, SEQ ID NO: 68, SEQ ID NO: 69, SEQ ID NO: 72, SEQ ID NO: 73, SEQ ID NO: 75, SEQ ID NO: 80, SEQ ID NO: 84, SEQ ID NO: 88, SEQ ID NO: 91, SEQ ID NO: 92, SEQ ID NO: 120, SEQ ID NO: 132, or SEQ ID NO: 139, or (c) at least about 96% identical to the nucleotide sequence set forth in SEQ ID NO: 4, SEQ ID NO: 6, SEQ ID NO: 10, SEQ ID NO: 13, SEQ ID NO: 16, SEQ ID NO: 17, SEQ ID NO: 19, SEQ ID NO: 28, SEQ ID NO: 33, SEQ ID NO: 34, SEQ ID NO: 35, SEQ ID NO: 40, SEQ ID NO: 50, SEQ ID NO: 58, SEQ ID NO: 62, SEQ ID NO: 64, SEQ ID NO: 65, SEQ ID NO: 67, SEQ ID NO: 68, SEQ ID NO: 69, SEQ ID NO: 72, SEQ ID NO: 73, SEQ ID NO: 75, SEQ ID NO: 80, SEQ ID NO: 84, SEQ ID NO: 88, SEQ ID NO: 91, SEQ ID NO: 92, SEQ ID NO: 120, SEQ ID NO: 132, or SEQ ID NO: 139.

In certain embodiments, each strain comprises a 16s RNA nucleotide sequence that is at least about 97% identical or 98.5% identical to the nucleotide sequence set forth in SEQ ID NO: 4, SEQ ID NO: 6, SEQ ID NO: 10, SEQ ID NO: 13, SEQ ID NO: 16, SEQ ID NO: 17, SEQ ID NO: 19, SEQ ID NO: 28, SEQ ID NO: 33, SEQ ID NO: 34, SEQ ID NO: 35, SEQ ID NO: 40, SEQ ID NO: 50, SEQ ID NO: 58, SEQ ID NO: 62, SEQ ID NO: 64, SEQ ID NO: 65, SEQ ID NO: 67, SEQ ID NO: 68, SEQ ID NO: 69, SEQ ID NO: 72, SEQ ID NO: 73, SEQ ID NO: 75, SEQ ID NO: 80, SEQ ID NO: 84, SEQ ID NO: 88, SEQ ID NO: 91, SEQ ID NO: 92, SEQ ID NO: 120, SEQ ID NO: 132, or SEQ ID NO: 139. In certain embodiments, each strain comprises a 16s RNA nucleotide sequence identical to the nucleotide sequence set forth in SEQ ID NO: SEQ ID NO: 4, SEQ ID NO: 6, SEQ ID NO: 10, SEQ ID NO: 13, SEQ ID NO: 16, SEQ ID NO: 17, SEQ ID NO: 19, SEQ ID NO: 28, SEQ ID NO: 33, SEQ ID NO: 34, SEQ ID NO: 35, SEQ ID NO: 40, SEQ ID NO: 50, SEQ ID NO: 58, SEQ ID NO: 62, SEQ ID NO: 64, SEQ ID NO: 65, SEQ ID NO: 67, SEQ ID NO: 68, SEQ ID NO: 69, SEQ ID NO: 72, SEQ ID NO: 73, SEQ ID NO: 75, SEQ ID NO: 80, SEQ ID NO: 84, SEQ ID NO: 88, SEQ ID NO: 91, SEQ ID NO: 92, SEQ ID NO: 120, SEQ ID NO: 132, or SEQ ID NO: 139.

In certain embodiments, the composition further comprises a fifth composition comprising Alistipes putredinis, Dialister succinatiphilus, Akkermansia muciniphila, Ruminococcus bromii, Dialister invisus, Bacteroides massiliensis, Bilophila wadsworthia, Holdemanella biformis, Parasutterella excrementihominis, Alistipes sp. FBI00180, Bacteroides coprocola, Alistipes sp. FBI00238, Alistipes putredinis, Eubacterium xylanophilum, Senegalimassilia anaerobia, or a functional equivalent thereof.

In certain embodiments, the composition further comprises FBI00022, FBI00049, FBI00068, FBI00069, FBI00152, FBI00165, FBI00171, FBI00175, FBI00177, FBI00180, FBI00182, FBI00238, FBI00269, FBI00274, FBI00281, or a functional equivalent thereof.

In certain embodiments, each strain comprises a 16s RNA nucleotide sequence that is (a) at least about 80% identical to the nucleotide sequence set forth in SEQ ID NO: 15, SEQ ID NO: 30, SEQ ID NO: 43, SEQ ID NO: 44, SEQ ID NO: 87, SEQ ID NO: 90, SEQ ID NO: 93, SEQ ID NO: 95, SEQ ID NO: 97, SEQ ID NO: 98, SEQ ID NO: 99, SEQ ID NO: 125, SEQ ID NO: 137, SEQ ID NO: 141, or SEQ ID NO: 144, (b) at least about 90% identical to the nucleotide sequence set forth in SEQ ID NO: 15, SEQ ID NO: 30, SEQ ID NO: 43, SEQ ID NO: 44, SEQ ID NO: 87, SEQ ID NO: 90, SEQ ID NO: 93, SEQ ID NO: 95, SEQ ID NO: 97, SEQ ID NO: 98, SEQ ID NO: 99, SEQ ID NO: 125, SEQ ID NO: 137, SEQ ID NO: 141, or SEQ ID NO: 144, or (c) at least about 96% identical to the nucleotide sequence set forth in SEQ ID NO: 15, SEQ ID NO: 30, SEQ ID NO: 43, SEQ ID NO: 44, SEQ ID NO: 87, SEQ ID NO: 90, SEQ ID NO: 93, SEQ ID NO: 95, SEQ ID NO: 97, SEQ ID NO: 98, SEQ ID NO: 99, SEQ ID NO: 125, SEQ ID NO: 137, SEQ ID NO: 141, or SEQ ID NO: 144.

In certain embodiments, each strain comprises a 16s RNA nucleotide sequence that is at least about 97% identical or 98.5% identical to the nucleotide sequence set forth in SEQ ID NO: 15, SEQ ID NO: 30, SEQ ID NO: 43, SEQ ID NO: 44, SEQ ID NO: 87, SEQ ID NO: 90, SEQ ID NO: 93, SEQ ID NO: 95, SEQ ID NO: 97, SEQ ID NO: 98, SEQ ID NO: 99, SEQ ID NO: 125, SEQ ID NO: 137, SEQ ID NO: 141, or SEQ ID NO: 144.

In certain embodiments, each strain comprises a 16s RNA nucleotide sequence identical to the nucleotide sequence set forth in SEQ ID NO: SEQ ID NO: 15, SEQ ID NO: 30, SEQ ID NO: 43, SEQ ID NO: 44, SEQ ID NO: 87, SEQ ID NO: 90, SEQ ID NO: 93, SEQ ID NO: 95, SEQ ID NO: 97, SEQ ID NO: 98, SEQ ID NO: 99, SEQ ID NO: 125, SEQ ID NO: 137, SEQ ID NO: 141, or SEQ ID NO: 144 Moreover, the present disclosure provides a microbial consortium comprising microbial strains set forth in Table 1, Table 2, Table 3, Table 4, Table 5, Table 6, Table 7, Table 8, Table 9, Table 10, Table 11, Table 12, Table 13, Table 14, Table 15, Table 16, Table 17, Table 18, Table 19, or a functional equivalent thereof.

The present disclosure also provides a microbial consortium comprising microbial strains set forth in Table 22 or a functional equivalent thereof.

In certain embodiments, each strain comprises a 16s RNA nucleotide sequence that is (a) at least about 80% identical to the nucleotide sequence set forth in SEQ ID NOs: 1-148, (b) at least about 90% identical to the nucleotide sequence set forth in SEQ ID NOs: 1-148, or (c) at least about 96% identical to the nucleotide sequence set forth in SEQ ID NOs: 1-148. In certain embodiments, each strain comprises a 16s RNA nucleotide sequence that is at least about 97% identical or 98.5% to the nucleotide sequence set forth in SEQ ID NOs: 1-148. In certain embodiments, each strain comprises a 16s RNA nucleotide sequence that is identical to the nucleotide sequence set forth in SEQ ID NOs: 1-148.

The present disclosure further provides a composition comprising a microbial consortium disclosed herein.

In certain embodiments, the composition disclosed herein is a pharmaceutical composition.

In certain embodiments, the composition comprises from about 5×10¹⁰ to about 5×10¹¹ viable cells. In certain embodiments, the composition comprises from about 5×10⁹ to about 5×10¹⁰ viable cells. In certain embodiments, the composition comprises from about 5×10¹¹ to about 5×10¹² viable cells. In certain embodiments, the composition comprises up to about 5×10¹² viable cells.

In certain embodiments, the composition comprises from about 10% to about 50% of oxalate-metabolizing microbial strains. In certain embodiments, the composition comprises from about 10% to about 50% of O. formigenes strains on a viable cell count basis. In certain embodiments, the composition comprises about 20% of O. formigenes strains on a viable cell count basis. In certain embodiments, the composition comprises about 30% of O. formigenes strains on a viable cell count basis. In certain embodiments, the composition comprises about 40% of O. formigenes strains on a viable cell count basis.

The present disclosure further provides a method of manufacturing the compositions or the microbial consortia disclosed herein. In certain embodiments, the method comprises obtaining and blending:

a) a first composition comprising Clostridium citroniae, Bacteroides salyersiae, Blautia obeum, Parabacteroides merdae, Parabacteroides distasonis, Anaerostipes hadrus, Lachnospiraceae sp. FBI00033, Eubacterium eligens, Bifidobacterium dentium, Blautia wexlerae, Fusicatenibacter saccharivorans, Bacteroides nordii, Dorea formicigenerans, Dorea longicatena, Bacteroides stercorirosoris, Bifidobacterium longum, Bacteroides kribbi, Lachnospiraceae sp. FBI00071, Bacteroides thetaiotaomicron, Clostridium clostridioforme, Clostridium scindens, Roseburia hominis, Clostridium fessum, Coprococcus comes, Blautia faecis, Hungatella hathewayi, Bacteroides stercoris, Collinsella aerofaciens, Hungatella effluvii, Bifidobacterium adolescentis, Bifidobacterium catenulatum, Lactobacillus rogosae, Bacteroides faecis, Bacteroides finegoldii, Clostridiaceae sp. FBI00191, Ruminococcus faecis, Lachnoclostridium pacaense, Clostridium bolteae, Longicatena caecimuris, Eggerthella lenta, Blautia massiliensis, Bacteroides xylanisolvens, Bacteroides vulgatus, Megasphaera massiliensis, Butyricimonas faecihominis, Eisenbergiella tayi, Acidaminococcus intestini, Emergencia timonensis, Bifidobacterium pseudocatenulatum, Eubacterium hallii, Anaerofustis stercorihominis, Eubacterium ventriosum, Blautia hydrogenotrophica, and Lachnospiraceae sp. FBI00290, or a functional equivalent thereof;

b) a second composition comprising Acutalibacter timonensis, Alistipes onderdonkii, Bacteroides uniformis, Eubacterium rectale, Alistipes timonensis, Bacteroides kribbi, Coprococcus eutactus, Bilophila wadsworthia, Bacteroides caccae, Alistipes shahii, Parasutterella excrementihominis, Paraprevotella clara, Sutterella wadsworthensis, Sutterella massiliensis, Porphyromonas asaccharolytica, Ruminococcus bromii, Monoglobus pectinilyticus, Ruminococcaceae sp. FBI00097, Gordonibacter pamelaeae, Bacteroides uniformis, Gordonibacter pamelaeae, Bacteroides fragilis, Phascolarctobacterium faecium, Monoglobus pectinilyticus, Clostridium aldenense, Ruthenibacterium lactatiformans, Bacteroides ovatus, Bifidobacterium bifidum, Anaerotruncus massiliensis, Clostridium aldenense, Sutterella wadsworthensis, Catabacter hongkongensis, Alistipes senegalensis, Ruminococcaceae sp. FBI00233, Alistipes shahii, Dielma fastidiosa, Eubacterium siraeum, Faecalibacterium prausnitzii, Turicibacter sanguinis, Eubacterium rectale, Bacteroides caccae, Methanobrevibacter smithii, Barnesiella intestinihominis, Alistipes onderdonkii, and Methanobrevibacter smithii, or a functional equivalent thereof, c) a third composition comprising Bifidobacterium adolescentis, Bifidobacterium longum, Bifidobacterium pseudocatenulatum, Bacteroides thetaiotaomicron, Coprococcus comes, Fusicatenibacter saccharivorans, Eggerthella lenta, Eubacterium eligens, Bacteroides xylanisolvens, Lactobacillus rogosae, Clostridium citroniae, Collinsella aerofaciens, Blautia obeum, Eggerthella lenta, Blautia wexlerae, Lachnoclostridium pacaense, Bacteroides vulgatus, Parabacteroides merdae, Dorea formicigenerans, Ruminococcus faecis, Roseburia hominis, Anaerostipes hadrus, Bifidobacterium adolescentis, Bifidobacterium pseudocatenulatum, Clostridium bolteae, Eisenbergiella tayi, Dorea longicatena, Eggerthella lenta, Bacteroides stercoris, Hungatella hathewayi, and Bacteroides xylanisolvens, or a functional equivalent thereof;

d) a fourth composition comprising Alistipes putredinis, Dialister succinatiphilus, Akkermansia muciniphila, Ruminococcus bromii, Dialister invisus, Bacteroides massiliensis, Bilophila wadsworthia, Holdemanella biformis, Parasutterella excrementihominis, Alistipes sp. FBI00180, Bacteroides coprocola, Alistipes sp. FBI00238, Alistipes putredinis, Eubacterium xylanophilum, and Senegalimassilia anaerobia, or a functional equivalent thereof;

e) a fifth composition comprising a first O. formigenes strain;

f) a sixth composition comprising a second O. formigenes strain; and/or

g) a seventh composition comprising a third O. formigenes strain.

In certain embodiments, the method comprises obtaining and blending:

a) a first composition comprising FBI00001, FBI00002, FBI00010, FBI00013, FBI00029, FBI00032, FBI00033, FBI00034, FBI00043, FBI00044, FBI00048, FBI00050, FBI00051, FBI00057, FBI00059, FBI00060, FBI00070, FBI00071, FBI00076, FBI00079, FBI00087, FBI00093, FBI00102, FBI00109, FBI00117, FBI00120, FBI00125, FBI00127, FBI00128, FBI00145, FBI00162, FBI00174, FBI00184, FBI00190, FBI00191, FBI00194, FBI00198, FBI00199, FBI00200, FBI00201, FBI00205, FBI00206, FBI00211, FBI00220, FBI00221, FBI00236, FBI00245, FBI00248, FBI00251, FBI00254, FBI00267, FBI00278, FBI00288, and FBI00290, or a functional equivalent thereof;

b) a second composition comprising FBI00004, FBI00012, FBI00015, FBI00018, FBI00019, FBI00021, FBI00038, FBI00040, FBI00046, FBI00061, FBI00066, FBI00075, FBI00077, FBI00080, FBI00081, FBI00085, FBI00092, FBI00097, FBI00099, FBI00112, FBI00132, FBI00137, FBI00140, FBI00149, FBI00151, FBI00176, FBI00189, FBI00197, FBI00208, FBI00212, FBI00224, FBI00226, FBI00229, FBI00233, FBI00235, FBI00237, FBI00243, FBI00244, FBI00258, FBI00260, FBI00263, FBI00270, FBI00273, FBI00277, and FBI00292, or a functional equivalent thereof;

c) a third composition comprising FBI00009, FBI00011, FBI00016, FBI00020, FBI00025, FBI00027, FBI00030, FBI00047, FBI00052, FBI00053, FBI00056, FBI00062, FBI00078, FBI00096, FBI00104, FBI00110, FBI00111, FBI00113, FBI00115, FBI00116, FBI00123, FBI00124, FBI00126, FBI00135, FBI00147, FBI00159, FBI00167, FBI00170, FBI00232, FBI00255, and FBI00271, or a functional equivalent thereof;

d) a fourth composition comprising FBI00022, FBI00049, FBI00068, FBI00069, FBI00152, FBI00165, FBI00171, FBI00175, FBI00177, FBI00180, FBI00182, FBI00238, FBI00269, FBI00274, and FBI00281, or a functional equivalent thereof;

e) a fifth composition comprising FBI00067 or a functional equivalent thereof;

f) a sixth composition comprising FBI00133 or a functional equivalent thereof, and/or

g) a seventh composition comprising FBI00289 or a functional equivalent thereof.

In certain embodiments, each strain comprises a 16s RNA nucleotide sequence that is (a) at least about 80% identical to the nucleotide sequence set forth in SEQ ID NOs: 1-148, (b) at least about 90% identical to the nucleotide sequence set forth in SEQ ID NOs: 1-148, or (c) at least about 96% identical to the nucleotide sequence set forth in SEQ ID NOs: 1-148. In certain embodiments, each strain comprises a 16s RNA nucleotide sequence that is at least about 97% identical or 98.5% identical to the nucleotide sequence set forth in SEQ ID NOs: 1-148. In certain embodiments, each strain comprises a 16s RNA nucleotide sequence identical to the nucleotide sequence set forth in SEQ ID NO: 1-148.

In certain embodiments, the fourth composition is obtained by growing microbes in presence of threonine. In certain embodiments, each composition comprises a lyoprotectant. In certain embodiments, each composition comprises maltodextrin, inulin, or a combination thereof. In certain embodiments, the maldextrin is at a concentration of about 8%. In certain embodiments, the inulin is at a concentration of about 0.5%. In certain embodiments, each composition is separately lyophilized.

In certain embodiments, the functional equivalent is based on the characteristics set forth in Table 24. In certain embodiments, the functional equivalent is based on the characteristics set forth in Table 34. In certain embodiments, the functional equivalent is based on the characteristics set forth in Table 35. In certain embodiments, the functional equivalent is based on the characteristics set forth in Table 36. In certain embodiments, the functional equivalent is based on the characteristics set forth in Tables 34-36.

In certain embodiments, the method comprises obtaining and blending microbes comprising a gene regulating oxalate degradation, oxalate resistance, formate metabolism, metabolism of macronutrients, production of microbial metabolites, cross-feeding activity, and/or mucin degradation. In certain embodiments, the method comprises obtaining and blending microbes that are known to protect against diseases and/or that are prevalent in healthy human gut. In certain embodiments, the method comprises obtaining and blending microbes that utilize carbon sources set forth in Table 35. In certain embodiments, each strain can optionally utilize a subset of the carbon sources set forth in Table 35.

In certain embodiments, each composition is prepared using inoculation density adjustment. In certain embodiments, each composition is cultured or has been cultured in presence of gas overlay. In certain embodiments, each composition is cultured or has been cultured in absence of gas sparging.

The present disclosure also provides a composition prepared by the methods of manufacturing disclosed herein.

Moreover, the present disclosure provides methods of treating hyperoxaluria in a subject in need thereof, reducing the risk of developing hyperoxaluria in a subject in need thereof, and/or reducing urinary oxalate in a subject in need thereof. In certain embodiments, the methods comprise administering an effective amount of the compositions or the microbial consortia disclosed herein.

In certain embodiments, the hyperoxaluria is a primary hyperoxaluria, a secondary hyperoxaluria, or an enteric hyperoxaluria. In certain embodiments, the secondary hyperoxaluria is associated with bowel resection surgery. In certain embodiments, the hyperoxaluria is enteric hyperoxaluria.

In certain embodiments, the methods further comprise administering at least one antibacterial agent, antiviral agent, antifungal agent, anti-inflammatory agent, immunosuppressive agent, prebiotic, or a combination thereof. In certain embodiments, the methods further comprise administering NOV-001, SYNB8802, OX-1, Lumasiran, Nedosiran, BBP-711, CNK-336, PBGENE-PH1, or a combination thereof. In certain embodiments, the methods further comprise administering a low oxalate diet, a high hydration diet, calcium supplements, or a combination thereof. In certain embodiments, the composition or the microbial consortium is administered orally.

In certain embodiments, the methods comprise administering a first dose of the compositions or the microbial consortia disclosed herein.

In certain embodiments, the methods further comprise administering an antibiotic treatment. In certain embodiments, the antibiotic treatment is administered for about 2 days, about 3 days, about 4 days, about 5 days, about 6 days, or about 7 days. In certain embodiments, the antibiotic is metronidazole, clarithromycin, or a combination thereof. In certain embodiments, the antibiotic treatment is completed 1 day before administering the first dose. In certain embodiments, the antibiotic treatment is completed 2 days before administering the first dose.

In certain embodiments, the methods further comprise administering a bowel preparation treatment. In certain embodiments, the bowel preparation treatment is administered to the subject after the antibiotic treatment. In certain embodiments, the bowel preparation treatment is administered before the first dose.

In certain embodiments, the first dose comprises an effective amount of the compositions or the microbial consortia. In certain embodiments, the first dose comprises about 10¹² viable cells. In certain embodiments, the first dose is administered for about 1 day. In certain embodiments, the first dose is administered for about 2 days.

In certain embodiments, the methods further comprise administering a second dose of the compositions or the microbial consortia. In certain embodiments, the second dose comprises an effective amount of the composition or the microbial consortium. In certain embodiments, the second dose comprises about 10¹¹ viable cells. In certain embodiments, the second dose is administered up to about 8 days. In certain embodiments, the second dose is administered up to about 10 days.

In certain embodiments, the first dose is administered orally. In certain embodiments, the second dose is administered orally.

The present disclosure also provides a kit comprising the compositions or the microbial consortia disclosed herein. In certain embodiments, the kit comprises a container comprising a desiccant. In certain embodiments, the container comprises anaerobic conditions. In certain embodiments, the container is a blister. In certain embodiments, the kit further comprises written instructions for administering the composition or microbial consortium.

The present disclosure also provides a method of culturing a microbial strain from the Akkermansia genus comprising contacting the strain with N-Acetylgalactosamine (GalNAc). In certain embodiments, the strain is Akkermansia muciniphilia.

The present disclosure also provides a microbial consortium comprising the functional properties set forth in Table 23, Table 24, Table 34, Table 35, Table 36. Finally, the present disclosure provides microbial consortia comprising FB-001 or a functional equivalent thereof.

### BRIEF DESCRIPTION OF THE DRAWINGS

FIGS. 1A, 1B, and 1C. FIG. 1A shows the reduction in urinary oxalate in mice fed a refined, sugary diet and gavaged with a Consortia described herein. FIG. 1B shows the reduction in urinary oxalate in mice fed a complex, grain-free diet and gavaged with a Consortia described herein. FIGS. 1A and 1B collectively show that the efficacy of reducing urinary oxalate using a Consortia described herein is independent of diet. FIG. 1C shows that the gastrointestinal microbiota present in an animal before treatment with a Consortia described herein does not affect the ability of the Consortia to reduce urinary oxalate levels.

FIGS. 2A and 2B. FIG. 2A shows an exemplary coculture experiment and FIG. 2B shows an exemplary coculture experiment that was modified to yield 100% strain detection following coculture.

FIGS. 3A and 3B. FIG. 3A shows the design of the DS buckets for a Consortia and FIG. 3B shows the yield of strains after coculture depending on the inoculum seed.

FIGS. 4A and 4B. FIGS. 4A and 4B show examples of different lyophilization excipients.

FIGS. 5A and 5B. FIGS. 5A and 5B show examples of different lyophilization excipients and reducing agents.

FIGS. 6A and 6B. FIGS. 6A and 6B show examples of different lyophilization excipients.

FIGS. 7A and 7B. FIG. 7A is a venn diagram showing the overlapping microbes of five representative consortia designed and disclosed herein. FIG. 7B shows the breakdown of the type of microbe in each of the 5 representative consortia.

FIGS. 8A and 8B. FIG. 8A shows a graph plotting the induction of EH in germ-free mice on different diets (control and oxalate diets as described in Example 4). FIG. 8B are graphs showing the relative abundance of O. formigenes and oxalate degradation.

FIG. 9. FIG. 9 shows oxalate and Ox:Cr ratios of Germ-free and “humanized” mice fed oxalate diets.

FIGS. 10A-10D. FIG. 10A shows the relative abundance of O. formigenes after dosing of Community I (Prevalence Based Community), Community II (2 Donor Community), Community III (Metabolism A Community), Community 4 (Metabolism B Community), or Community 5 (Diversity Community). FIG. 10B shows the species richness of mice fed an Ox36 diet followed by dosing of one of the five representative consortia. FIG. 10C shows the species richness of mice fed a 5021+0.875% Ox diet followed by dosing of one of the five representative consortia. FIG. 10D shows the species richness of humanized mice dosed with one of the five representative consortia.

FIGS. 11A and 11B. FIGS. 11A and 11B show the schematics of the experimental designs of the studies described in Example 5.

FIG. 12. FIG. 12 shows that YCFAC+GalNAc is not able to support the growth of Akkermansia.

FIG. 13. FIG. 13 shows that Threonine supports the growth of Akkermansia in the absence of GalNAc.

FIG. 14. FIG. 14 shows a diagram of the coculture method of manufacture.

FIG. 15. FIG. 15 shows an overview of the strain isolation and purification process, RCB banking, and RCB identity/purity testing.

FIG. 16. FIG. 16 shows a method for generation of master cell banks (MCB).

FIG. 17. FIG. 17 shows a phylogenetic tree indicating the taxonomic composition of the FB-001 Consortium.

FIGS. 18A-18C. FIGS. 18A-18C show a table summarizing the strains and species of the microbial consortia disclosed herein.

FIGS. 19A and 19B. FIG. 19A shows the effect FB-001 has on reducing gut permeability and FIG. 19B shows the ability of FB-001 to produce short chain fatty acids (SCFA) at a level that is comparable to a normal, healthy gut. Butyrate, a SCFA, is important because it supports gastrointestinal epithelial cell health, energy metabolism and cell signaling to improve barrier function.

FIGS. 20A-20D. FIGS. 20A-20D show that FB-001 reduces urinary oxalate (UrOx) by 35-68% in vivo across different diets (i.e., the ability of FB-001 and DS1-DS4 to reduce urinary oxalate independent of diet and existing microbiota). FIG. 20A shows a depiction of the study design. FIG. 20B shows the Oxalate:Creatinine ratio of mice fed a complex, grain-based diet. FIG. 20C shows the Oxalate:Creatinine ratio of mice fed a refined, high-sugar diet. FIG. 20D shows the Oxalate:Creatinine ratio of humanized mice.

FIG. 21. FIG. 21 shows a comparison done by mathematical modelling of the oxalate degradation rate (per cell) of FB-001 compared to Novome's WW554 and WW626 hyperoxaluria drug products and Synlogics 8802 drug product). The data shows that FB-001 is able to achieve oxalate consumption at a significantly higher rate than the other drug products and suggests it will be more effective at treating hyperoxaluria in subjects in need thereof.

FIG. 22. FIG. 22 shows the manufacturing process used for O. formigenes in the production of the Consortia described herein. Furthermore, DS5-DS7 (i.e., the three O. formigenes drug substances) of FB-001 used this manufacturing process for GMP and non-GMP manufacture.

FIG. 23. FIG. 23 shows the manufacturing process used for DS1 in the production of the Consortia described herein. Furthermore, DS1 of FB-001 used this manufacturing process for GMP and non-GMP manufacture.

FIG. 24. FIG. 24 shows the manufacturing process used for DS2 in the production of the Consortia described herein. Furthermore, DS2 of FB-001 used this manufacturing process for GMP and non-GMP manufacture.

FIG. 25. FIG. 25 shows the manufacturing process used for DS3 in the production of the Consortia described herein. Furthermore, DS3 of FB-001 used this manufacturing process for GMP and non-GMP manufacture.

FIG. 26. FIG. 26 shows the manufacturing process used for DS4 in the production of the Consortia described herein. Furthermore, DS4 of FB-001 used this manufacturing process for GMP and non-GMP manufacture.

### DETAILED DESCRIPTION

The present disclosure relates to compositions and methods for reducing oxalate in a subject. For clarity of description, and not by way of limitation, this section is divided into the following subsections:
(a) Definitions;
(b) Biological Niches;
(c) Physical Compartments;
(d) Metabolic Compartments;
(e) Consortia;
(f) Active Microbes;
(g) Oxalate-Metabolizing Active Microbes;
(h) Supportive Community of Microbes;
(j) Consortia Design;
(k) Methods of Preparation;
(i) Pharmaceutical Compositions;
(l) Functionally Equivalent and Identical Drug Products;
(m) Therapeutic Applications;
(n) Methods of Treating Hyperoxaluria;
(o) Dosages;
(p) Combination Therapy;
(q) Kits; and
(r) Exemplary Embodiments.

### Definitions

Unless defined otherwise, all technical and scientific terms used herein have the meaning commonly understood by a person skilled in the art. The following references provide one of skill with a general definition of many of the terms used in the presently disclosed subject matter: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

It is understood that aspects and embodiments of the present disclosure described herein include “comprising,” “consisting,” and “consisting essentially of” aspects and embodiments.

The terms “comprises” and “comprising” are intended to have the broad meaning ascribed to them in U.S. Patent Law and can mean “includes,” “including” and the like.

To facilitate an understanding of the present disclosure, a number of terms and phrases are defined below.

The term “a” and “an” as used herein mean “one or more” and include the plural unless the context is appropriate

As used herein, the term “active microbes” refers to microbes that express sufficient amounts of one or more than one metabolic enzyme to metabolize a substrate that causes or contributes to disease in an animal.

As used herein, the term “biomass,” refers to the total mass of one or more than one microbe, or consortium in a given area or volume.

As used herein, the terms “microbial consortia” and “microbial consortium” are used interchangeably and refer to a mixture of two or more isolated microbial strains that are expanded in culture, wherein one microbial strain in the mixture has a beneficial or desired effect on another microbial strain in the mixture.

As used herein, the term “Consortia” is used as a capitalized term to refer to one or more of the microbial consortia described herein.

As used herein, the term “gastrointestinal engraftment” or “engraft” or “engraftment” refers to the establishment of one or more than one microbe, or microbial consortium, in one or more than one niche of the gastrointestinal tract that, prior to administration of the one or more than one microbe, or microbial consortium, is absent in the one or more than one microbe, or microbial consortium.

Gastrointestinal engraftment may be transient, or may be persistent.

As used herein, the term “effective amount” refers to an amount sufficient to achieve a beneficial or desired result. In certain embodiments, an effective amount can be improved gastrointestinal engraftment of one or more than one of the plurality of active microbes, increased biomass of one or more than one of the plurality of active microbes, increased metabolism of the first metabolic substrate, or improved longitudinal stability).

As used herein, the term “fermenting microbe” refers to a microbe that expresses sufficient amounts of one or more than one enzyme to catalyze a fermentation reaction in a gastrointestinal niche.

As used herein, the term “longitudinal stability” refers to the ability of one or more than one microbe, or microbial consortium to remain engrafted and metabolically active in one of more than one niche of the gastrointestinal tract despite transient or long-term environmental changes to the gastrointestinal niche.

As used herein, the term “metabolism,” “metabolize,” “metabolization,” or variants thereof refers to the biochemical conversion of a metabolic substrate to a metabolic product. In certain embodiments, metabolization includes isomerization.

As used herein, the term “microbe” or “microbiota” refers to a microbial organism including, but not limited to, bacteria, archaea, protozoa, and unicellular fungi.

As used herein, the term “pharmaceutical composition” refers to the combination of an active agent with a carrier, inert or active, making the composition especially suitable for therapeutic use in vivo or ex vivo.

As used herein, the term “pharmaceutically acceptable carrier” refers to any of the standard pharmaceutical carriers, such as phosphate buffered saline solution, water, emulsions (e.g., such as oil/water or water/oil emulsions), and various types of wetting agents. The compositions also can include stabilizers and preservatives. For examples of carriers, stabilizers, and adjuvants, see e.g., Martin, Remington's Pharmaceutical Sciences, 15^{th} Ed. Mack Publ. Co., Easton, Pa. [1975].

As used herein, “significantly” or “significant” refers to a change or alteration in a measurable parameter to a statistically significant degree as determined in accordance with an appropriate statistically relevant test. For example, in certain non-limiting embodiments, a change or alteration is significant if it is statistically significant in accordance with, e.g., a Student's t-test, chi-square, or Mann Whitney test.

As used herein, the term “standardized substrate metabolization assay” refers to an experimental assay known to persons of ordinary skill in the art used to quantify the amount of substrate converted to a metabolic product.

As used herein, the term “subject” refers to an organism to be treated by the microbial consortium and compositions described herein. Such organisms preferably include, but are not limited to, mammals (e.g., murines, simians, equines, bovines, porcines, canines, felines, and the like), and more preferably include humans.

As used herein, the term “supportive community” refers to one or more than one microbial strain that, when administered with an active microbe, enhances one or more than one characteristic of the active microbe selected from the group consisting of gastrointestinal engraftment, biomass, metabolic substrate metabolism, and longitudinal stability.

As used herein, the term “synthesizing microbe” refers to a microbe that expresses sufficient amounts of one or more than one enzyme to catalyze the combination of one or more than one metabolite produced by an active microbe, and one or more than one fermentation product produced by a fermenting microbe in a gastrointestinal niche.

The term percent “identity” or “sequence identity,” in the context of two or more nucleic acid or polypeptide sequences, refer to two or more sequences or subsequences that have a specified percentage of nucleotides or amino acid residues that are the same, when compared and aligned for maximum correspondence, as measured using one of the sequence comparison algorithms described below (e.g., BLASTP and BLASTN or other algorithms available to persons of skill) or by visual inspection.

Depending on the application, the percent “identity” can exist over a region of the sequence being compared, e.g., over a functional domain, or, alternatively, exist over the full length of the two sequences to be compared.

For sequence comparison, typically one sequence acts as a reference sequence to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by visual inspection (see generally Ausubel et al., infra).

One example of an algorithm that is suitable for determining percent sequence identity and sequence similarity is the BLAST algorithm, which is described in Altschul et al., J. Mol. Biol. 215:403-410 (1990). Software for performing BLAST analyses is publicly available through the National Center for Biotechnology Information (www.ncbi.nlm.nih.gov/).

When used in reference to 16S rRNA sequences, a “sequence identity” of at least 97% indicates that two microbial strains are likely to belong to the same species, whereas 16S rRNA sequences having less than 97% sequence identity indicate that two microbial strains likely belong to different species, and 16S rRNA sequences having less than 95% sequence identity indicates that two microbial strains likely belong to distinct genera (Stackebrandt E., and Goebel, B. M., Int J Syst Bact, 44 (1994) 846-849.).

As used herein, the terms “functional equivalent” or “functionally equivalent” refers to microbes, microbial consortia, and compositions that share similar or identical role (e.g., metabolism of oxalate). For example, without any limitation, two different microbial consortia that can catalyze high concentration of oxalate are functional equivalent to each other. In certain non-limiting embodiments, a microbe, a microbial consortium, and a composition that is functional equivalent can be based on the characteristic outlined in Table 24 (see Example section).

Throughout the description, where compositions are described as having, including, or comprising specific components, or where processes and methods are described as having, including, or comprising specific steps, it is contemplated that, additionally, there are compositions of the present disclosure that consist essentially of, or consist of, the recited components, and that there are processes and methods according to the present disclosure that consist essentially of, or consist of, the recited processing steps.

As a general matter, compositions specifying a percentage are by weight unless otherwise specified. Further, if a variable is not accompanied by a definition, then the previous definition of the variable controls.

### Biological Niches

Disclosed herein are microbial consortia for administration to an animal comprising a plurality of active microbes which metabolize a first metabolic substrate which causes or contributes to disease in the animal. The microbial consortia disclosed herein further comprise an effective amount of a supportive community of microbes that metabolize one or more than one metabolite produced by the plurality of active microbes, and wherein the one or more than one metabolite inhibits metabolism of the plurality of active microbes. These microbial consortia are advantageous in having enhanced characteristics when administered to an animal as compared to administration of the plurality of active microbes alone. Enhanced characteristics of the microbial consortia include one or more of improved gastrointestinal engraftment, increased biomass, increased metabolism of the first metabolic substrate, and improved longitudinal stability.

The present disclosure provides microbial consortia capable of engrafting into one or more than one niche of a gastrointestinal tract where it is capable of metabolizing a substrate that causes or contributes to disease in an animal. These niches comprise specific microbial communities whose composition varies according to a number of environmental factors including, but not limited to, the particular physical compartment of the gastrointestinal tract inhabited by a microbial community, the chemical and physicochemical properties of the environment inhabited, the metabolic substrate composition of the environment inhabited, and other co-inhabiting microbial species.

### Physical Compartments

A gastrointestinal tract comprises a number of physical compartments. For example, the human gastrointestinal tract includes the oral cavity, pharynx, esophagus, stomach, small intestine (duodenum, jejunum, ileum), cecum, large intestine (ascending colon, transverse colon, descending colon), and rectum. The pancreas, liver, gallbladder, and associated ducts, additionally comprise compartments of the human gastrointestinal tract. Each of these compartments has, for example, variable anatomical shape and dimension, aeration, water content, levels of mucus secretion, luminal presence of antimicrobial peptides, and presence or absence of peristaltic motility. Furthermore, the different gastrointestinal compartments vary in their pH. In humans, the pH of the oral cavity, upper stomach, lower stomach, duodenum, jejunum, ileum, and colon range from 6.5-7.5, 4.0-6.5, 1.5-4.0, 7.0-8.5, 4.0-7.0, and 4.0-7.0, respectively. Compartments of the gastrointestinal tract also differ in their levels of oxygenation which are subject to large degrees of fluctuation. For example, the luminal partial pressure of oxygen in the stomach of mice has been measured to be approximately 58 mm Hg, while the luminal partial pressure of oxygen in the distal sigmoid colon has been measured to be approximately 3 mm Hg (He et al., 1999). Oxygen levels of the gastrointestinal tract are highly determinative of the biochemical pathways utilized by commensal microbes. For example, commensal bacteria utilize aerobic respiration at oxygen concentrations above 5 mbar of O₂, anaerobic respiration between 1-5 mbar of O₂, and fermentation at O₂ concentrations below 1 mbar. The sensitivity of microbes to O₂ levels and their ability to carry out metabolic reactions under aerobic and/or anaerobic conditions influences which microbial species engraft in a particular gastrointestinal compartment.

### Metabolic Compartments

In addition to the various physical and chemical environments contributing to a gastrointestinal niche, different niches comprise different metabolic substrates.

Metabolic substrates that may be present in a gastrointestinal niche may include, but are not limited to, oxalate, fructan, inulin, glucuronoxylan, arabinoxylan, glucomannan, β-mannan, dextran, starch, arabinan, xyloglucan, galacturonan, β-glucan, galactomannan, rhamnogalacturonan I, rhamnogalacturonan II, arabinogalactan, mucin O-linked glycans, yeast α-mannan, yeast β-glucan, chitin, alginate, porphyrin, laminarin, carrageenan, agarose, alternan, levan, xanthan gum, galactooligosaccharides, hyaluronan, chondrointin sulfate, dermatan sulfate, heparin sulfate, keratan sulfate, phenylalanine, tyrosine, tryptophan, leucine, valine, isoleucine, glycine, proline, asparagine, glutamine, aspartate, glutamate, cysteine, lysine, arginine, serine, methionine, alanine, arginine, histidine, ornithine, citrulline, carnitine, hydroxyproline, cholic acid, chenodeoxycholic acid, taurochenodeoxycholic acid, glycochenodeoxycholic acid, cholesterol, cinnamic acid, coumaric acid, sinapinic acid, ferulic acid, caffeic acid, quinic acid, chlorogenic acid, catechin, epicatechin, gallic acid, pyrogallol, catechol, quercetin, myricetin, campherol, luteolin, apigenin, naringenin, and hesperidin.

### Consortia

The present disclosure provides Consortia comprising a plurality of active microbes and an effective amount of a supportive community of microbes. In certain embodiments, the Consortia comprises the microbiota listed in any of Tables 1-19. Tables 1-19 are provided below:

TABLE 1
Consortia I

| | | | | |
|---|---|---|---|---|
| Acidaminococcus | Bacteroides | Blautia | Coprococcus | Holdemanella |
| intestini | stercoris | hydrogenotrophica | eutactus | biformis |
| Akkermansia | Bacteroides | Blautia luti | Coprococcus | Holdemanella |
| muciniphila | stercoris | | eutactus | biformis |
| Alistipes | Bacteroides | Blautia luti | Desulfovibrio | Hungatella |
| finegoldii | thetaiotaomicron | | desulfuricans | hathewayi |
| Alistipes | Bacteroides | Blautia luti | Desulfovibrio | Hungatella |
| onderdonkii | thetaiotaomicron | | desulfuricans | hathewayi |
| Alistipes | Bacteroides | Blautia obeum | Dialister invisus | Neglecta |
| onderdonkii | thetaiotaomicron | | | timonensis |
| Alistipes | Bacteroides | Blautia obeum | Dorea | Oxalobacter |
| onderdonkii | uniformis | | formicigenerans | formigenes |
| Alistipes | Bacteroides | Blautia obeum | Dorea | Oxalobacter |
| putredinis | uniformis | | formicigenerans | formigenes |
| Alistipes | Bacteroides | Blautia obeum | Dorea | Oxalobacter |
| putredinis | uniformis | | formicigenerans | formigenes |
| Alistipes | Bacteroides | Blautia wexlerae | Dorea | Parabacteroides |
| senegalensis | uniformis | | longi catena | distasonis |
| Alistipes | Bacteroides | Blautia wexlerae | Dorea | Parabacteroides |
| senegalensis | vulgatus | | longi catena | distasonis |
| Alistipes shahii | Bacteroides | Blautia wexlerae | Dorea | Parabacteroides |
| | vulgatus | | longi catena | distasonis |
| Alistipes shahii | Bacteroides | Clostridium | Eggerthella lenta | Parabacteroides |
| | vulgatus | aldenense | | merdae |
| Alistipes shahii | Bacteroides | Clostridium | Eggerthella lenta | Parabacteroides |
| | vulgatus | aldenense | | merdae |
| Alistipes | Bacteroides | Clostridium | Eggerthella lenta | Parabacteroides |
| timonensis | xylanisolvens | amygdalinum | | merdae |
| Anaerofustis | Bacteroides | Clostridium | Eggerthella lenta | Paraprevotella |
| stercorihominis | xylanisolvens | bolteae | | clara |
| Anaerostipes | Bacteroides | Clostridium | Eubacterium | Parasutterella |
| hadrus | xylanisolvens | bolteae | eligens | excrementihominis |
| Anaerostipes | Bifidobacterium | Clostridium | Eubacterium | Parasutterella |
| hadrus | adolescentis | citroniae | eligens | excrementihominis |
| Anaerotruncus | Bifidobacterium | Clostridium | Eubacterium | Roseburia hominis |
| colihominis | adolescentis | citroniae | eligens | |
| Bacteroides | Bifidobacterium | Clostridium | Eubacterium | Roseburia hominis |
| caccae | catenulatum | scindens | eligens | |
| Bacteroides | Bifidobacterium | Clostridium | Eubacterium | Roseburia hominis |
| caccae | dentium | symbiosum | hallii | |
| Bacteroides | Bifidobacterium | Clostridium | Eubacterium | Roseburia hominis |
| cellulosilyticus | longum | symbiosum | rectale | |
| Bacteroides | Bifidobacterium | Clostridium | Eubacterium | Ruminococcus |
| coprocola | longum | symbiosum | rectale | bromii |
| Bacteroides | Bifidobacterium | Collinsella | Eubacterium | Ruminococcus |
| finegoldii | longum | aerofaciens | rectale | bromii |
| Bacteroides | Bifidobacterium | Collinsella | Eubacterium | Ruminococcus |
| fragilis | longum | aerofaciens | rectale | bromii |
| Bacteroides | Bifidobacterium | Collinsella | Eubacterium | Ruminococcus |
| massiliensis | pseudocatenulatum | aerofaciens | siraeum | bromii |
| Bacteroides | Bifidobacterium | Collinsella | Eubacterium | Ruminococcus |
| massiliensis | pseudocatenulatum | aerofaciens | ventriosum | faecis |
| Bacteroides | Bifidobacterium | Coprococcus | Eubacterium | Ruminococcus |
| nordii | pseudocatenulatum | comes | xylanophilum | faecis |
| Bacteroides | Blautia faecis | Coprococcus | Faecalibacterium | Turicibacter |
| oleiciplenus | | comes | prausnitzii | sanguinis |
| Bacteroides | Blautia faecis | Coprococcus | Faecalibacterium | |
| ovatus | | comes | prausnitzii | |
| Bacteroides | Blautia faecis | Coprococcus | Gordonibacter | |
| salyersiae | | comes | pamelaeae | |
| Bacteroides | Blautia faecis | Coprococcus | Gordonibacter | |
| stercoris | | eutactus | pamelaeae | |

TABLE 2
Consortia II

| | | | | |
|---|---|---|---|---|
| Akkermansia | Bacteroides | Clostridium | Eggerthella lenta | Oxalobacter |
| muciniphila | vulgatus | amygdalinum | | formigenes |
| Alistipes | Bifidobacterium | Clostridium | Eubacterium | Parabacteroides |
| onderdonkii | dentium | citroniae | eligens | distasonis |
| Alistipes | Bifidobacterium | Clostridium | Eubacterium | Parabacteroides |
| putredinis | faecale | citroniae | eligens | distasonis |
| Alistipes shahii | Bifidobacterium | Clostridium | Eubacterium | Parabacteroides |
| | longum | scindens | rectale | merdae |
| Alistipes | Bifidobacterium | Clostridium | Eubacterium | Paraprevotella clara |
| timonensis | longum | symbiosum | rectale | |
| Bacteroides | Bifidobacterium | Collinsella | Faecalibacterium | Parasutterella |
| caccae | pseudocatenulatum | aerofaciens | prausnitzii | excrementihominis |
| Bacteroides | Bifidobacterium | Coprococcus | Fusicatenibacter | Phascolarctobacterium |
| koreensis | pseudocatenulatum | comes | saccharivorans | faecium |
| Bacteroides | Bifidobacterium | Coprococcus | Fusicatenibacter | Phascolarctobacterium |
| kribbi | pseudocatenulatum | eutactus | saccharivorans | faecium |
| Bacteroides | Blautia faecis | Desulfovibrio | Gordonibacter | Phascolarctobacterium |
| kribbi | | desulfuricans | pamelaeae | faecium |
| Bacteroides | Blautia faecis | Dialister | Lachnoclostridium | Roseburia hominis |
| nordii | | succinatiphilus | pacaense | |
| Bacteroides | Blautia obeum | Dorea | Lachnospira | Ruminococcus bromii |
| ovatus | | formicigenerans | pectinoschiza | |
| Bacteroides | Blautia obeum | Dorea | Monoglobus | Ruminococcus bromii |
| salyersiae | | longicatena | pectinilyticus | |
| Bacteroides | Blautia obeum | Eggerthella | Neglecta | Ruminococcus faecis |
| thetaiotaomicron | | lenta | timonensis | |
| Bacteroides | Blautia wexlerae | Eggerthella | Oxalobacter | Sutterella massiliensis |
| thetaiotaomicron | | lenta | formigenes | |
| Bacteroides | Blautia wexlerae | Eggerthella | Oxalobacter | Sutterella |
| uniformis | | lenta | formigenes | wadsworthensis |

TABLE 3
Consortia III

| | | | | |
|---|---|---|---|---|
| Akkermansia | Bacteroides | Clostridium | Eubacterium | Parabacteroides |
| muciniphila | vulgatus | scindens | rectale | merdae |
| Anaerotruncus | Bacteroides | Clostridium | Eubacterium | Ruminococcus |
| colihominis | vulgatus | symbiosum | rectale | bromii |
| Bacteroides | Bacteroides | Clostridium | Eubacterium | Ruminococcus |
| caccae | vulgatus | symbiosum | rectale | bromii |
| Bacteroides | Bifidobacterium | Clostridium | Eubacterium | Ruminococcus |
| caccae | adolescentis | symbiosum | rectale | bromii |
| Bacteroides | Bifidobacterium | Collinsella | Eubacterium | Ruminococcus |
| cellulosilyticus | adolescentis | aerofaciens | siraeum | bromii |
| Bacteroides | Bifidobacterium | Collinsella | Faecalibacterium | Sutterella |
| fragilis | bifidum | aerofaciens | prausnitzii | wadsworthensis |
| Bacteroides | Bifidobacterium | Collinsella | Gordonibacter | Sutterella |
| massiliensis | bifidum | aerofaciens | pamelaeae | wadsworthensis |
| Bacteroides | Bifidobacterium | Collinsella | Gordonibacter | Sutterella |
| massiliensis | catenulatum | aerofaciens | pamelaeae | wadsworthensis |
| Bacteroides | Bifidobacterium | Coprococcus | Hydrogeno- | Bacteroides |
| salyersiae | dentium | comes | anaerobacterium | vulgatus |
| | | | saccharovorans | |
| Bacteroides | Bifidobacterium | Coprococcus | Lachnospiraceae | Clostridium |
| stercoris | longum | comes | sp. | citroniae |
| Bacteroides | Bifidobacterium | Coprococcus | Lactonifactor | Eggerthella lenta |
| stercoris | longum | comes | longoviformis | |
| Bacteroides | Bifidobacterium | Desulfovibrio | Neglecta | Parabacteroides |
| stercoris | longum | desulfuricans | timonensis | merdae |
| Bacteroides | Bifidobacterium | Desulfovibrio | Oxalobacter | Parabacteroides |
| thetaiotaomicron | longum | desulfuricans | formigenes | merdae |
| Bacteroides | Bifidobacterium | Dorea longicatena | Oxalobacter | Eggerthella lenta |
| thetaiotaomicron | pseudocatenulatum | | formigenes | |
| Bacteroides | Bifidobacterium | Dorea longicatena | Oxalobacter | Clostridium |
| thetaiotaomicron | pseudocatenulatum | | formigenes | citroniae |
| Bacteroides | Bifidobacterium | Dorea longicatena | Parabacteroides | Bacteroides |
| uniformis | pseudocatenulatum | | distasonis | uniformis |
| Bacteroides | Citrobacter | Eggerthella lenta | Parabacteroides | |
| uniformis | freundii | | distasonis | |
| Bacteroides | Clostridium | Eggerthella lenta | Parabacteroides | |
| uniformis | amygdalinum | | distasonis | |

TABLE 4
Consortia IV

| | | | | |
|---|---|---|---|---|
| Alistipes | Bacteroides | Clostridium | Dorea longicatena | Oxalobacter |
| finegoldii | vulgatus | bolteae | | formigenes |
| Alistipes | Bacteroides | Clostridium | Eggerthella lenta | Parabacteroides |
| putredinis | vulgatus | bolteae | | merdae |
| Alistipes | Bacteroides | Clostridium | Eggerthella lenta | Parabacteroides |
| putredinis | xylanisolvens | citroniae | | merdae |
| Anaerotruncus | Bacteroides | Clostridium | Eggerthella lenta | Parabacteroides |
| colihominis | xylanisolvens | citroniae | | merdae |
| Bacteroides | Bacteroides | Clostridium | Eggerthella lenta | Ruminococcus |
| caccae | xylanisolvens | scindens | | bromii |
| Bacteroides | Bifidobacterium | Clostridium | Eubacterium | Ruminococcus |
| cellulosilyticus | bifidum | symbiosum | eligens | bromii |
| Bacteroides | Bifidobacterium | Clostridium | Eubacterium | Ruminococcus |
| coprocola | bifidum | symbiosum | eligens | bromii |
| Bacteroides | Bifidobacterium | Clostridium | Eubacterium | Ruminococcus |
| fragilis | catenulatum | symbiosum | eligens | bromii |
| Bacteroides | Bifidobacterium | Collinsella | Eubacterium | Sutterella |
| ovatus | dentium | aerofaciens | eligens | wadsworthensis |
| Bacteroides | Bifidobacterium | Collinsella | Eubacterium | |
| salyersiae | longum | aerofaciens | hallii | |
| Bacteroides | Bifidobacterium | Collinsella | Eubacterium | |
| stercoris | longum | aerofaciens | rectale | |
| Bacteroides | Bifidobacterium | Collinsella | Eubacterium | |
| stercoris | longum | aerofaciens | siraeum | |
| Bacteroides | Bifidobacterium | Coprococcus | Eubacterium | |
| stercoris | longum | comes | ventriosum | |
| Bacteroides | Bifidobacterium | Coprococcus | Faecalibacterium | |
| thetaiotaomicron | pseudocatenulatum | eutactus | prausnitzii | |
| Bacteroides | Bifidobacterium | Coprococcus | Faecalibacterium | |
| thetaiotaomicron | pseudocatenulatum | eutactus | prausnitzii | |
| Bacteroides | Bifidobacterium | Coprococcus | Hungatella | |
| thetaiotaomicron | pseudocatenulatum | eutactus | hathewayi | |
| Bacteroides | Blautia | Desulfovibrio | Hungatella | |
| uniformis | hydrogenotrophica | desulfuricans | hathewayi | |
| Bacteroides | Blautia obeum | Desulfovibrio | Neglecta | |
| uniformis | | desulfuricans | timonensis | |
| Bacteroides | Blautia obeum | Dorea | Oxalobacter | |
| vulgatus | | formicigenerans | formigenes | |
| Bacteroides | Clostridium | Dorea longicatena | Oxalobacter | |
| vulgatus | amygdalinum | | formigenes | |

TABLE 5
Consortia V

| | | | | |
|---|---|---|---|---|
| Acidaminococcus | Bacteroides | Clostridium | Eubacterium | Phascolarctobacter |
| intestini | uniformis | citroniae | ventriosum | ium faecium |
| Akkermansia | Bacteroides | Clostridium | Eubacterium | Phascolarctobacter |
| muciniphila | vulgatus | clostridioforme | siraeum | ium faecium |
| Alistipes | Bacteroides | Clostridium | Eubacterium | Phocea |
| finegoldii | vulgatus | scindens | xylanophilum | massiliensis |
| Alistipes | Bacteroides | Clostridium | Faecalibacterium | Phocea |
| onderdonkii | xylanisolvens | swellfunianum | prausnitzii | massiliensis |
| Alistipes | Bacteroides | Clostridium | Faecalibacterium | Porphyromonas |
| onderdonkii | xylanisolvens | symbiosum | prausnitzii | asaccharolytica |
| Alistipes | Barnesiella | Clostridium | Faecalicatena | Porphyromonas |
| putredinis | intestinihominis | symbiosum | contorta | asaccharolytica |
| Alistipes | Bifidobacterium | Collinsella | Fusicatenibacter | Roseburia hominis |
| putredinis | adolescentis | aerofaciens | saccharivorans | |
| Alistipes | Bifidobacterium | Collinsella | Fusicatenibacter | Roseburia hominis |
| senegalensis | adolescentis | aerofaciens | saccharivorans | |
| Alistipes | Bifidobacterium | Coprococcus | Gordonibacter | Ruminococcus |
| senegalensis | bifidum | comes | pamelaeae | bromii |
| Alistipes shahii | Bifidobacterium | Coprococcus | Gordonibacter | Ruminococcus |
| | bifidum | comes | pamelaeae | bromii |
| Alistipes | Bifidobacterium | Coprococcus | Holdemanella | Ruminococcus |
| timonensis | catenulatum | eutactus | biformis | faecis |
| Anaerofustis | Bifidobacterium | Coprococcus | Holdemanella | Ruminococcus |
| stercorihominis | dentium | eutactus | biformis | faecis |
| Anaerostipes | Bifidobacterium | Desulfovibrio | Hungatella | Ruthenibacterium |
| hadrus | faecale | desulfuricans | effluvii | lactatiformans |
| Anaerostipes | Bifidobacterium | Desulfovibrio | Hungatella | Senegalimassilia |
| hadrus | longum | desulfuricans | hathewayi | anaerobia |
| Anaerotruncus | Bifidobacterium | Dialister invisus | Hungatella | Sutterella |
| colihominis | longum | | hathewayi | massiliensis |
| Bacteroides | Bifidobacterium | Dialister | Hydrogeno- | Sutterella |
| caccae | pseudocatenulatum | succinatiphilus | anaerobacterium | wadsworthensis |
| | | | saccharovorans | |
| Bacteroides | Bifidobacterium | Dielma | Lachnoclostridium | Sutterella |
| caccae | pseudocatenulatum | fastidiosa | pacaense | wadsworthensis |
| Bacteroides | Bifidobacterium | Dorea | Lachnoclostridium | Turicibacter |
| coprocola | pseudocatenulatum | formicigenerans | pacaense | sanguinis |
| Bacteroides | Blautia faecis | Dorea | Lachnospira | |
| faecis | | formicigenerans | pectinoschiza | |
| Bacteroides | Blautia faecis | Dorea | Lachnospira | |
| finegoldii | | longicatena | pectinoschiza | |
| Bacteroides | Blautia | Dorea | Lactonifactor | |
| fragilis | hydrogenotrophica | longicatena | longoviformis | |
| Bacteroides | Blautia luti | Eggerthella lenta | Longicatena | |
| koreensis | | | caecimuris | |
| Bacteroides | Blautia obeum | Eggerthella lenta | Megasphaera | |
| koreensis | | | massiliensis | |
| Bacteroides | Blautia obeum | Eggerthella lenta | Monoglobus | |
| kribbi | | | pectinilyticus | |
| Bacteroides | Blautia wexlerae | Eggerthella lenta | Monoglobus | |
| kribbi | | | pectinilyticus | |
| Bacteroides | Blautia wexlerae | Eisenbergiella | Neglecta | |
| massiliensis | | tayi | timonensis | |
| Bacteroides | Butyricimonas | Eisenbergiella | Oxalobacter | |
| nordii | faecihominis | tayi | formigenes | |
| Bacteroides | Catabacter | Emergencia | Oxalobacter | |
| oleiciplenus | hongkongensis | timonensis | formigenes | |
| Bacteroides | Citrobacter | Eubacterium | Oxalobacter | |
| ovatus | freundii | eligens | formigenes | |
| Bacteroides | Clostridium | Eubacterium | Parabacteroides | |
| salyersiae | aldenense | eligens | distasonis | |
| Bacteroides | Clostridium | Eubacterium | Parabacteroides | |
| stercoris | aldenense | hallii | merdae | |
| Bacteroides | Clostridium | Eubacterium | Parabacteroides | |
| stercoris | amygdalinum | oxidoreducens | merdae | |
| Bacteroides | Clostridium | Eubacterium | Paraprevotella | |
| thetaiotaomicron | bolteae | rectale | clara | |
| Bacteroides | Clostridium | Eubacterium | Parasutterella | |
| thetaiotaomicron | bolteae | rectale | excrementihominis | |
| Bacteroides | Clostridium | Eubacterium | Parasutterella | |
| uniformis | citroniae | ruminantium | excrementihominis | |

TABLE 6
Consortia VI

| | | | | |
|---|---|---|---|---|
| Acidaminococcus | Bacteroides | Butyricimonas sp. | Enterococcus | Longicatena |
| intestini | stercorirosoris | FBI00158 | casse liflavus | caecimuris |
| Acidaminococcus | Bacteroides | Catabacter | Enterococcus | Megasphaera |
| intestini | stercoris | hongkongensis | casse liflavus | massiliensis |
| Acutalibacter | Bacteroides | Citrobacter | Enterococcus | Methanobrevibacter |
| timonensis | stercoris | portucalensis | durans | smithii |
| Akkermansia | Bacteroides | Clostridiaceae sp. | Enterococcus | Monoglobus |
| muciniphila | thetaiotaomicron | FBI00191 | durans | pectinilyticus |
| Alistipes | Bacteroides | Clostridium | Enterococcus | Monoglobus |
| onderdonkii | thetaiotaomicron | aldenense | durans | pectinilyticus |
| Alistipes | Bacteroides | Clostridium | Enterococcus | Oxalobacter |
| onderdonkii | uniformis | aldenense | faecalis | formigenes |
| Alistipes | Bacteroides | Clostridium | Enterococcus | Oxalobacter |
| putredinis | uniformis | bolteae | faecium | formigenes |
| Alistipes | Bacteroides | Clostridium | Escherichia | Oxalobacter |
| putredinis | vulgatus | bolteae | flexneri | formigenes |
| Alistipes | Bacteroides | Clostridium | Eubacterium | Parabacteroides |
| senegalensis | vulgatus | citroniae | eligens | distasonis |
| Alistipes shahii | Bacteroides | Clostridium | Eubacterium | Parabacteroides |
| | xylanisolvens | citroniae | eligens | distasonis |
| Alistipes shahii | Bacteroides | Clostridium | Eubacterium | Parabacteroides |
| | xylanisolvens | clostridioforme | hallii | merdae |
| Alistipes sp. | Bacteroides | Clostridium | Eubacterium | Parabacteroides |
| FBI00180 | xylanisolvens | fessum | rectale | merdae |
| Alistipes sp. | Barnesiella | Clostridium | Eubacterium | Paraprevotella |
| FBI00238 | intestinihominis | fessum | rectale | clara |
| Alistipes | Bifidobacterium | Clostridium | Eubacterium | Parasutterella |
| timonensis | adolescentis | scindens | siraeum | excrementihominis |
| Anaerofustis | Bifidobacterium | Collinsella | Eubacterium | Parasutterella |
| stercorihominis | adolescentis | aerofaciens | ventriosum | excrementihominis |
| Anaerostipes | Bifidobacterium | Collinsella | Eubacterium | Phascolarctobacterium |
| hadrus | adolescentis | aerofaciens | xylanophilum | faecium |
| Anaerostipes | Bifidobacterium | Coprococcus | Faecalibacterium | Phascolarctobacterium |
| hadrus | adolescentis | comes | prausnitzii | faecium |
| Anaerotruncus | Bifidobacterium | Coprococcus | Faecalibacterium | Porphyromonas |
| massiliensis | bifidum | comes | prausnitzii | asaccharolytica |
| Bacteroides | Bifidobacterium | Coprococcus | Faecalicatena | Porphyromonas |
| caccae | bifidum | eutactus | contorta | asaccharolytica |
| Bacteroides | Bifidobacterium | Dialister invisus | Fusicatenibacter | Roseburia hominis |
| caccae | catenulatum | | saccharivorans | |
| Bacteroides | Bifidobacterium | Coprococcus | Fusicatenibacter | Roseburia hominis |
| cellulosilyticus | dentium | eutactus | saccharivorans | |
| Bacteroides | Bifidobacterium | Dialister | Gordonibacter | Ruminococcaceae |
| cellulosilyticus | longum | succinatiphilus | pamelaeae | sp. FBI00097 |
| Bacteroides | Bifidobacterium | Dialister | Gordonibacter | Ruminococcaceae |
| coprocola | longum | succinatiphilus | pamelaeae | sp. FBI00097 |
| Bacteroides dorei | Bifidobacterium | Dielma fastidiosa | Holdemanella | Ruminococcaceae |
| | pseudocatenulatum | | biformis | sp. FBI00233 |
| Bacteroides dorei | Bifidobacterium | Dorea | Holdemanella | Ruminococcus |
| | pseudocatenulatum | formicigenerans | biformis | bromii |
| Bacteroides faecis | Bilophila | Dorea | Hungatella | Ruminococcus |
| | wadsworthia | formicigenerans | effluvii | bromii |
| Bacteroides | Bilophila | Dorea longicatena | Hungatella | Ruminococcus |
| finegoldii | wadsworthia | | effluvii | faecis |
| Bacteroides | Blautia faecis | Dorea longicatena | Hungatella | Ruminococcus |
| fragilis | | | effluvii | faecis |
| Bacteroides kribbi/ | Blautia faecis | Eggerthella lenta | Lachnoclostridium | Ruthenibacterium |
| Bacteroides | | | pacaense | lactatiformans |
| koreensis species | | | | |
| cluster | | | | |
| Bacteroides kribbi/ | Blautia | Eggerthella lenta | Lachnoclostridium | Senegalimassilia |
| Bacteroides | hydrogenotrophica | | pacaense | anaerobia |
| koreensis species | | | | |
| cluster | | | | |
| Bacteroides kribbi/ | Blautia luti | Eisenbergiella tayi | Lachnospiraceae | Sutterella |
| Bacteroides | | | sp. FBI00033 | massiliensis |
| koreensis species | | | | |
| cluster | | | | |
| Bacteroides | Blautia | Emergencia | Lachnospiraceae | Sutterella |
| massiliensis | massiliensis | timonensis | sp. FBI00071 | wadsworthensis |
| Bacteroides | Blautia obeum | Eisenbergiella tayi | Lachnospiraceae | Sutterella |
| massiliensis | | | sp. FBI00150 | wadsworthensis |
| Bacteroides nordii | Blautia obeum | Enterobacter | Lachnospiraceae | Turicibacter |
| | | himalayensis | sp. FBI00290 | sanguinis |
| Bacteroides ovatus | Blautia wexlerae | Enterobacter | Lactobacillus | |
| | | hormaechei | rogosae | |
| Bacteroides | Blautia wexlerae | Enterococcus | Lactobacillus | |
| salyersiae | | casseliflavus | rogosae | |
| Bacteroides | Butyricimonas | Enterococcus | Lactonifactor | |
| salyersiae | faecihominis | casseliflavus | longoviformis | |

TABLE 7
Consortia VII

| | | | | |
|---|---|---|---|---|
| Acidaminococcus | Bacteroides | Citrobacter | Eubacterium | Oxalobacter |
| intestini | thetaiotaomicron | portucalensis | eligens | formigenes |
| Acutalibacter | Bacteroides | Clostridiaceae | Eubacterium | Parabacteroides |
| timonensis | uniformis | sp. FBI00191 | hallii | distasonis |
| Akkermansia | Bacteroides | Clostridium | Eubacterium | Parabacteroides |
| muciniphila | uniformis | aldenense | rectale | merdae |
| Alistipes | Bacteroides | Clostridium | Eubacterium | Parabacteroides |
| onderdonkii | vulgatus | aldenense | rectale | merdae |
| Alistipes | Bacteroides | Clostridium | Eubacterium | Paraprevotella clara |
| onderdonkii | vulgatus | bolteae | siraeum | |
| Alistipes putredinis | Bacteroides | Clostridium | Eubacterium | Parasutterella |
| | xylanisolvens | bolteae | ventriosum | excrementihominis |
| Alistipes putredinis | Bacteroides | Clostridium | Faecalibacterium | Parasutterella |
| | xylanisolvens | citroniae | prausnitzii | excrementihominis |
| Alistipes | Bacteroides | Clostridium | Eubacterium | Phascolarctobacterium |
| senegalensis | xylanisolvens | citroniae | xylanophilum | faecium |
| Alistipes shahii | Barnesiella | Clostridium | Faecalibacterium | Phascolarctobacterium |
| | intestinihominis | clostridioforme | prausnitzii | faecium |
| Alistipes sp. | Bifidobacterium | Clostridium | Faecalicatena | Porphyromonas |
| FBI00180 | adolescentis | fessum | contorta | asaccharolytica |
| Alistipes sp. | Bifidobacterium | Clostridium | Fusicatenibacter | Porphyromonas |
| FBI00238 | adolescentis | fessum | saccharivorans | asaccharolytica |
| Alistipes | Bifidobacterium | Clostridium | Fusicatenibacter | Roseburia hominis |
| timonensis | adolescentis | scindens | saccharivorans | |
| Anaerofustis | Bifidobacterium | Collinsella | Gordonibacter | Roseburia hominis |
| stercorihominis | bifidum | aerofaciens | pamelaeae | |
| Anaerostipes | Bifidobacterium | Collinsella | Gordonibacter | Ruminococcaceae sp. |
| hadrus | bifidum | aerofaciens | pamelaeae | FBI00097 |
| Anaerostipes | Bifidobacterium | Coprococcus | Holdemanella | Ruminococcaceae sp. |
| hadrus | catenulatum | comes | biformis | FBI00097 |
| Anaerotruncus | Bifidobacterium | Coprococcus | Holdemanella | Ruminococcaceae sp. |
| massiliensis | dentium | comes | biformis | FBI00233 |
| Bacteroides caccae | Bifidobacterium | Coprococcus | Hungatella | Ruminococcus bromii |
| | longum | eutactus | effluvii | |
| Bacteroides caccae | Bifidobacterium | Coprococcus | Hungatella | Ruminococcus bromii |
| | longum | eutactus | effluvii | |
| Bacteroides | Bifidobacterium | Dialister invisus | Hungatella | Ruminococcus faecis |
| coprocola | pseudocatenulatum | | effluvii | |
| Bacteroides faecis | Bifidobacterium | Dialister | Lachnoclostridium | Ruminococcus faecis |
| | pseudocatenulatum | succinatiphilus | pacaense | |
| Bacteroides | Bifidobacterium | Dielma | Lachnoclostridium | Ruthenibacterium |
| finegoldii | pseudocatenulatum | fastidiosa | pacaense | lactatiformans |
| Bacteroides fragilis | Bilophila | Dorea | Lachnospiraceae | Senegalimassilia |
| | wadsworthia | formicigenerans | sp. FBI00033 | anaerobia |
| Bacteroides kribbi/ | Bilophila | Dorea | Lachnospiraceae | Sutterella massiliensis |
| Bacteroides | wadsworthia | formicigenerans | sp. FBI00071 | |
| koreensis species | | | | |
| cluster | | | | |
| Bacteroides kribbi/ | Blautia faecis | Dorea | Lachnospiraceae | Sutterella |
| Bacteroides | | longicatena | sp. FBI00290 | wadsworthensis |
| koreensis species | | | | |
| cluster | | | | |
| Bacteroides kribbi/ | Blautia faecis | Dorea | Lactobacillus | Sutterella |
| Bacteroides | | longicatena | rogosae | wadsworthensis |
| koreensis species | | | | |
| cluster | | | | |
| Bacteroides | Blautia | Eggerthella | Lactobacillus | Turicibacter sanguinis |
| massiliensis | hydrogenotrophica | lenta | rogosae | |
| Bacteroides nordii | Blautia massiliensis | Eggerthella | Lactonifactor | |
| | | lenta | longoviformis | |
| Bacteroides ovatus | Blautia obeum | Eggerthella | Longicatena | |
| | | lenta | caecimuris | |
| Bacteroides | Blautia obeum | Eggerthella | Megasphaera | |
| salyersiae | | lenta | massiliensis | |
| Bacteroides | Blautia wexlerae | Eisenbergiella | Monoglobus | |
| stercorirosoris | | tayi | pectinilyticus | |
| Bacteroides | Blautia wexlerae | Eisenbergiella | Monoglobus | |
| stercoris | | tayi | pectinilyticus | |
| Bacteroides | Butyricimonas | Emergencia | Oxalobacter | |
| stercoris | faecihominis | timonensis | formigenes | |
| Bacteroides | Catabacter | Eubacterium | Oxalobacter | |
| thetaiotaomicron | hongkongensis | eligens | formigenes | |

TABLE 8
Consortia VIII

| | | | | |
|---|---|---|---|---|
| Acidaminococcus | Bacteroides | Butyricimonas | Eisenbergiella | Monoglobus |
| intestini | thetaiotaomicron | faecihominis | tayi | pectinilyticus |
| Acutalibacter | Bacteroides | Catabacter | Emergencia | Monoglobus |
| timonensis | thetaiotaomicron | hongkongensis | timonensis | pectinilyticus |
| Akkermansia | Bacteroides | Citrobacter | Eubacterium | Oxalobacter |
| muciniphila | uniformis | portucalensis | eligens | formigenes |
| Alistipes | Bacteroides | Clostridiaceae | Eubacterium | Oxalobacter |
| onderdonkii | uniformis | sp. FBI00191 | eligens | formigenes |
| Alistipes | Bacteroides | Clostridium | Eubacterium | Oxalobacter |
| onderdonkii | vulgatus | aldenense | hallii | formigenes |
| Alistipes putredinis | Bacteroides | Clostridium | Eubacterium | Parabacteroides |
| | vulgatus | aldenense | rectale | distasonis |
| Alistipes putredinis | Bacteroides | Clostridium | Eubacterium | Parabacteroides |
| | xylanisolvens | bolteae | rectale | merdae |
| Alistipes | Bacteroides | Clostridium | Eubacterium | Parabacteroides |
| senegalensis | xylanisolvens | bolteae | siraeum | merdae |
| Alistipes shahii | Bacteroides | Clostridium | Eubacterium | Paraprevotella clara |
| | xylanisolvens | citroniae | ventriosum | |
| Alistipes sp. | Barnesiella | Clostridium | Faecalibacterium | Parasutterella |
| FBI00180 | intestinihominis | citroniae | prausnitzii | excrementihominis |
| Alistipes sp. | Bifidobacterium | Clostridium | Eubacterium | Parasutterella |
| FBI00238 | adolescentis | clostridioforme | xylanophilum | excrementihominis |
| Alistipes | Bifidobacterium | Clostridium | Faecalibacterium | Phascolarctobacterium |
| timonensis | adolescentis | fessum | prausnitzii | faecium |
| Anaerofustis | Bifidobacterium | Clostridium | Faecalicatena | Phascolarctobacterium |
| stercorihominis | adolescentis | fessum | contorta | faecium |
| Anaerostipes | Bifidobacterium | Clostridium | Fusicatenibacter | Porphyromonas |
| hadrus | bifidum | scindens | saccharivorans | asaccharolytica |
| Anaerostipes | Bifidobacterium | Collinsella | Fusicatenibacter | Porphyromonas |
| hadrus | bifidum | aerofaciens | saccharivorans | asaccharolytica |
| Anaerotruncus | Bifidobacterium | Collinsella | Gordonibacter | Roseburia hominis |
| massiliensis | catenulatum | aerofaciens | pamelaeae | |
| Bacteroides caccae | Bifidobacterium | Coprococcus | Gordonibacter | Roseburia hominis |
| | dentium | comes | pamelaeae | |
| Bacteroides caccae | Bifidobacterium | Coprococcus | Holdemanella | Ruminococcaceae sp. |
| | longum | comes | biformis | FBI00097 |
| Bacteroides | Bifidobacterium | Coprococcus | Holdemanella | Ruminococcaceae sp. |
| coprocola | longum | eutactus | biformis | FBI00097 |
| Bacteroides faecis | Bifidobacterium | Coprococcus | Hungatella effluvii | Ruminococcaceae sp. |
| | pseudocatenulatum | eutactus | | FBI00233 |
| Bacteroides | Bifidobacterium | Dialister invisus | Hungatella effluvii | Ruminococcus bromii |
| finegoldii | pseudocatenulatum | | | |
| Bacteroides fragilis | Bifidobacterium | Dialister | Hungatella effluvii | Ruminococcus bromii |
| | pseudocatenulatum | succinatiphilus | | |
| Bacteroides kribbi/ | Bilophila | Dielma | Lachnoclostridium | Ruminococcus faecis |
| Bacteroides | wadsworthia | fastidiosa | pacaense | |
| koreensis species | | | | |
| cluster | | | | |
| Bacteroides kribbi/ | Bilophila | Dorea | Lachnoclostridium | Ruminococcus faecis |
| Bacteroides | wadsworthia | formicigenerans | pacaense | |
| koreensis species | | | | |
| cluster | | | | |
| Bacteroides kribbi/ | Blautia faecis | Dorea | Lachnospiraceae | Ruthenibacterium |
| Bacteroides | | formicigenerans | sp. FBI00033 | lactatiformans |
| koreensis species | | | | |
| cluster | | | | |
| Bacteroides | Blautia faecis | Dorea | Lachnospiraceae | Senegalimassilia |
| massiliensis | | longicatena | sp. FBI00071 | anaerobia |
| Bacteroides nordii | Blautia | Dorea | Lachnospiraceae | Sutterella massiliensis |
| | hydrogenotrophica | longicatena | sp. FBI00290 | |
| Bacteroides ovatus | Blautia | Eggerthella | Lactobacillus | Sutterella |
| | massiliensis | lenta | rogosae | wadsworthensis |
| Bacteroides | Blautia obeum | Eggerthella | Lactobacillus | Sutterella |
| salyersiae | | lenta | rogosae | wadsworthensis |
| Bacteroides | Blautia obeum | Eggerthella | Lactonifactor | Turicibacter sanguinis |
| stercorirosoris | | lenta | longoviformis | |
| Bacteroides | Blautia wexlerae | Eggerthella | Longicatena | |
| stercoris | | lenta | caecimuris | |
| Bacteroides | Blautia wexlerae | Eisenbergiella | Megasphaera | |
| stercoris | | tayi | massiliensis | |

TABLE 9
Consortia IX

| | | | | |
|---|---|---|---|---|
| Acidaminococcus | Bacteroides | Butyricimonas | Eubacterium | Neglecta timonensis |
| intestini | thetaiotaomicron | faecihominis | eligens | |
| Akkermansia | Bacteroides | Catabacter | Eubacterium | Oxalobacter |
| muciniphila | thetaiotaomicron | hongkongensis | eligens | formigenes |
| Alistipes | Bacteroides | Clostridiaceae | Eubacterium hallii | Oxalobacter |
| onderdonkii | uniformis | sp. FBI00191 | | formigenes |
| Alistipes | Bacteroides | Clostridiales sp. | Eubacterium rectale | Oxalobacter |
| onderdonkii | uniformis | FBI00377 | | formigenes |
| Alistipes putredinis | Bacteroides | Clostridium | Eubacterium rectale | Parabacteroides |
| | vulgatus | aldenense | | distasonis |
| Alistipes putredinis | Bacteroides | Clostridium | Eubacterium | Parabacteroides |
| | vulgatus | aldenense | siraeum | distasonis |
| Alistipes | Bacteroides | Clostridium | Eubacterium | Parabacteroides |
| senegalensis | xylanisolvens | bolteae | ventriosum | merdae |
| Alistipes shahii | Bacteroides | Clostridium | Eubacterium | Parabacteroides |
| | xylanisolvens | bolteae | xylanophilum | merdae |
| Alistipes shahii | Bacteroides | Clostridium | Faecalibacterium | Paraprevotella clara |
| | xylanisolvens | citroniae | prausnitzii | |
| Alistipes sp. | Barnesiella | Clostridium | Fusicatenibacter | Parasutterella |
| FBI00180 | intestinihominis | citroniae | saccharivorans | excrementihominis |
| Alistipes sp. | Bifidobacterium | Clostridium | Fusicatenibacter | Parasutterella |
| FBI00238 | adolescentis | clostridioforme | saccharivorans | excrementihominis |
| Alistipes | Bifidobacterium | Clostridium | Gordonibacter | Phascolarctobacterium |
| timonensis | adolescentis | fessum | pamelaeae | faecium |
| Anaerofustis | Bifidobacterium | Clostridium | Gordonibacter | Porphyromonas |
| stercorihominis | adolescentis | scindens | pamelaeae | asaccharolytica |
| Anaerostipes | Bifidobacterium | Collinsella | Holdemanella | Porphyromonas |
| hadrus | bifidum | aerofaciens | biformis | asaccharolytica |
| Anaerostipes | Bifidobacterium | Collinsella | Hungatella effluvii | Roseburia hominis |
| hadrus | catenulatum | aerofaciens | | |
| Anaerotruncus | Bifidobacterium | Coprococcus | Hungatella effluvii | Roseburia hominis |
| massiliensis | dentium | comes | | |
| Bacteroides caccae | Bifidobacterium | Coprococcus | Hungatella effluvii | Ruminococcaceae sp. |
| | longum | comes | | FBI00082 FBI00097 |
| Bacteroides caccae | Bifidobacterium | Coprococcus | Lachnoclostridium | Ruminococcaceae sp. |
| | longum | eutactus | pacaense | FBI00233 |
| Bacteroides | Bifidobacterium | Dialister invisus | Lachnoclostridium | Ruminococcus bromii |
| coprocola | pseudocatenulatum | | pacaense | |
| Bacteroides faecis | Bifidobacterium | Dialister | Lachnospiraceae sp. | Ruminococcus bromii |
| | pseudocatenulatum | succinatiphilus | FBI00033 | |
| Bacteroides | Bifidobacterium | Dielma | Lachnospiraceae sp. | Ruminococcus faecis |
| finegoldii | pseudocatenulatum | fastidiosa | FBI00071 | |
| Bacteroides fragilis | Bilophila | Dorea | Lachnospiraceae sp. | Ruminococcus faecis |
| | wadsworthia | formicigenerans | FBI00290 | |
| Bacteroides kribbi/ | Bilophila | Dorea | Lactobacillus | Ruthenibacterium |
| Bacteroides | wadsworthia | formicigenerans | rogosae | lactatiformans |
| koreensis species | | | | |
| cluster | | | | |
| Bacteroides kribbi/ | Blautia faecis | Dorea | Lactobacillus | Senegalimassilia |
| Bacteroides | | longicatena | rogosae | anaerobia |
| koreensis species | | | | |
| cluster | | | | |
| Bacteroides | Blautia faecis | Dorea | Longicatena | Sutterella massiliensis |
| massiliensis | | longicatena | caecimuris | |
| Bacteroides nordii | Blautia | Eggerthella | Megasphaera | Sutterella |
| | hydrogenotrophica | lenta | massiliensis | wadsworthensis |
| Bacteroides ovatus | Blautia massiliensis | Eggerthella | Methanobrevibacter | Sutterella |
| | | lenta | smithii | wadsworthensis |
| Bacteroides | Blautia obeum | Eggerthella | Methanobrevibacter | Turicibacter sanguinis |
| salyersiae | | lenta | smithii | |
| Bacteroides | Blautia obeum | Eggerthella | Monoglobus | |
| stercorirosoris | | lenta | pectinilyticus | |
| Bacteroides | Blautia wexlerae | Eisenbergiella | Monoglobus | |
| stercoris | | tayi | pectinilyticus | |
| Bacteroides | Blautia wexlerae | Eisenbergiella | Neglecta timonensis | |
| stercoris | | tayi | | |

TABLE 10
Consortia X

| | | | | |
|---|---|---|---|---|
| Acidaminococcus | Bacteroides | Butyricimonas | Eubacterium eligens | Monoglobus |
| intestini | thetaiotaomicron | faecihominis | | pectinilyticus |
| Akkermansia | Bacteroides | Catabacter | Eubacterium eligens | Neglecta timonensis |
| muciniphila | thetaiotaomicron | hongkongensis | | |
| Alistipes | Bacteroides | Clostridiaceae | Eubacterium hallii | Neglecta timonensis |
| onderdonkii | uniformis | sp. FBI00191 | | |
| Alistipes | Bacteroides | Clostridiales sp. | Eubacterium rectale | Oxalobacter |
| onderdonkii | uniformis | FBI00377 | | formigenes |
| Alistipes putredinis | Bacteroides | Clostridium | Eubacterium rectale | Oxalobacter |
| | vulgatus | aldenense | | formigenes |
| Alistipes putredinis | Bacteroides | Clostridium | Eubacterium | Oxalobacter |
| | vulgatus | aldenense | siraeum | formigenes |
| Alistipes | Bacteroides | Clostridium | Eubacterium | Parabacteroides |
| senegalensis | xylanisolvens | bolteae | ventriosum | distasonis |
| Alistipes shahii | Bacteroides | Clostridium | Eubacterium | Parabacteroides |
| | xylanisolvens | bolteae | xylanophilum | distasonis |
| Alistipes shahii | Bacteroides | Clostridium | Faecalibacterium | Parabacteroides |
| | xylanisolvens | citroniae | prausnitzii | merdae |
| Alistipes sp. | Barnesiella | Clostridium | Faecalibacterium | Parabacteroides |
| FBI00180 | intestinihominis | citroniae | prausnitzii | merdae |
| Alistipes sp. | Bifidobacterium | Clostridium | Fusicatenibacter | Paraprevotella clara |
| FBI00238 | adolescentis | clostridioforme | saccharivorans | |
| Alistipes | Bifidobacterium | Clostridium | Fusicatenibacter | Parasutterella |
| timonensis | adolescentis | fessum | saccharivorans | excrementihominis |
| Anaerofustis | Bifidobacterium | Clostridium | Gordonibacter | Parasutterella |
| stercorihominis | adolescentis | scindens | pamelaeae | excrementihominis |
| Anaerostipes | Bifidobacterium | Collinsella | Gordonibacter | Phascolarctobacterium |
| hadrus | bifidum | aerofaciens | pamelaeae | faecium |
| Anaerostipes | Bifidobacterium | Collinsella | Holdemanella | Porphyromonas |
| hadrus | catenulatum | aerofaciens | biformis | asaccharolytica |
| Anaerotruncus | Bifidobacterium | Coprococcus | Holdemanella | Porphyromonas |
| massiliensis | dentium | comes | biformis | asaccharolytica |
| Bacteroides caccae | Bifidobacterium | Coprococcus | Hungatella effluvii | Roseburia hominis |
| | longum | comes | | |
| Bacteroides caccae | Bifidobacterium | Coprococcus | Hungatella effluvii | Roseburia hominis |
| | longum | eutactus | | |
| Bacteroides | Bifidobacterium | Dialister invisus | Hungatella effluvii | Ruminococcaceae sp. |
| coprocola | pseudocatenulatum | | | FBI00082 FBI00097 |
| Bacteroides faecis | Bifidobacterium | Dialister | Lachnoclostridium | Ruminococcaceae sp. |
| | pseudocatenulatum | succinatiphilus | pacaense | FBI00233 |
| Bacteroides | Bifidobacterium | Dielma | Lachnoclostridium | Ruminococcus bromii |
| finegoldii | pseudocatenulatum | fastidiosa | pacaense | |
| Bacteroides fragilis | Bilophila | Dorea | Lachnospiraceae sp. | Ruminococcus bromii |
| | wadsworthia | formicigenerans | FBI00033 | |
| Bacteroides kribbi/ | Bilophila | Dorea | Lachnospiraceae sp. | Ruminococcus faecis |
| Bacteroides | wadsworthia | formicigenerans | FBI00071 | |
| koreensis species | | | | |
| cluster | | | | |
| Bacteroides kribbi/ | Blautia faecis | Dorea | Lachnospiraceae sp. | Ruminococcus faecis |
| Bacteroides | | longicatena | FBI00290 | |
| koreensis species | | | | |
| cluster | | | | |
| Bacteroides | Blautia faecis | Dorea | Lactobacillus | Ruthenibacterium |
| massiliensis | | longicatena | rogosae | lactatiformans |
| Bacteroides nordii | Blautia | Eggerthella | Lactobacillus | Senegalimassilia |
| | hydrogenotrophica | lenta | rogosae | anaerobia |
| Bacteroides ovatus | Blautia massiliensis | Eggerthella | Longicatena | Sutterella massiliensis |
| | | lenta | caecimuris | |
| Bacteroides | Blautia obeum | Eggerthella | Megasphaera | Sutterella |
| salyersiae | | lenta | massiliensis | wadsworthensis |
| Bacteroides | Blautia obeum | Eggerthella | Methanobrevibacter | Sutterella |
| stercorirosoris | | lenta | smithii | wadsworthensis |
| Bacteroides | Blautia wexlerae | Eisenbergiella | Methanobrevibacter | Turicibacter sanguinis |
| stercoris | | tayi | smithii | |
| Bacteroides | Blautia wexlerae | Eisenbergiella | Monoglobus | |
| stercoris | | tayi | pectinilyticus | |

TABLE 11
Consortia XI

| | | | | |
|---|---|---|---|---|
| Acidaminococcus | Bifidobacterium | Fusicatenibacter | Bacteroides | Clostridium |
| intestini | longum | saccharivorans | xylanisolvens | bolteae |
| Akkermansia | Bilophila | Gordonibacter | Turicibacter | Collinsella |
| muciniphila | wadsworthia | pamelaeae | sanguinis | aerofaciens |
| Alistipes | Blautia | Hungatella effluvii | Bifidobacterium | Coprococcus |
| onderdonkii | hydrogenotrophica | | adolescentis | comes |
| Alistipes | Blautia | Lachnoclostridium | Bifidobacterium | Dorea |
| putredinis | massiliensis | pacaense | pseudocatenulatum | formicigenerans |
| Alistipes | Blautia obeum | Lachnospiraceae sp. | Blautia faecis | Dorea longicatena |
| senegalensis | | FBI00033 | | |
| Alistipes shahii | Blautia wexlerae | Lachnospiraceae sp. | Clostridium | Eggerthella lenta |
| | | FBI00071 | citroniae | |
| Alistipes sp. | Butyricimonas | Lachnospiraceae sp. | Faecalibacterium | Eggerthella lenta |
| FBI00180 | faecihominis | FBI00290 | prausnitzii | |
| Alistipes sp. | Catabacter | Lactobacillus rogosae | Holdemanella | Eggerthella lenta |
| FBI00238 | hongkongensis | | biformis | |
| Alistipes | Clostridiaceae sp. | Longicatena | Bacteroides | Eisenbergiella tayi |
| timonensis | FBI00191 | caecimuris | xylanisolvens | |
| Anaerofustis | Clostridiales sp. | Megasphaera | Bifidobacterium | Eubacterium |
| stercorihominis | FBI00377 | massiliensis | adolescentis | eligens |
| Anaerostipes | Clostridium | Methanobrevibacter | Bifidobacterium | Eubacterium |
| hadrus | aldenense | smithii | pseudocatenulatum | rectale |
| Anaerotruncus | Clostridium | Monoglobus | Blautia faecis | Fusicatenibacter |
| massiliensis | bolteae | pectinilyticus | | saccharivorans |
| Bacteroides | Clostridium | Neglecta timonensis | Alistipes | Gordonibacter |
| caccae | clostridioforme | | onderdonkii | pamelaeae |
| Bacteroides | Clostridium | Oxalobacter | Clostridium | Hungatella effluvii |
| coprocola | fessum | formigenes | citroniae | |
| Bacteroides | Clostridium | Oxalobacter | Alistipes | Hungatella effluvii |
| faecis | scindens | formigenes | putredinis | |
| Bacteroides | Collinsella | Oxalobacter | Alistipes shahii | Lachnoclostridium |
| finegoldii | aerofaciens | formigenes | | pacaense |
| Bacteroides | Coprococcus | Parabacteroides | Anaerostipes | Lactobacillus |
| fragilis | comes | distasonis | hadrus | rogosae |
| Bacteroides | Coprococcus | Parabacteroides | Bacteroides | Methanobrevibacter |
| kribbi/ | eutactus | merdae | caccae | smithii |
| Bacteroides | | | | |
| koreensis species | | | | |
| cluster | | | | |
| Bacteroides | Dialister invisus | Paraprevotella clara | Bacteroides kribbi/ | Monoglobus |
| massiliensis | | | Bacteroides | pectinilyticus |
| | | | koreensis species | |
| | | | cluster | |
| Bacteroides | Dialister | Parasutterella | Bacteroides | Neglecta |
| nordii | succinatiphilus | excrementihominis | stercoris | timonensis |
| Bacteroides | Dielma fastidiosa | Phascolarctobacterium | Bacteroides | Parabacteroides |
| ovatus | | faecium | thetaiotaomicron | distasonis |
| Bacteroides | Dorea | Porphyromonas | Bacteroides | Parabacteroides |
| salyersiae | formicigenerans | asaccharolytica | uniformis | merdae |
| Bacteroides | Dorea longicatena | Roseburia hominis | Bacteroides | Parasutterella |
| stercorirosoris | | | vulgatus | excrementihominis |
| Bacteroides | Eggerthella lenta | Ruminococcaceae sp. | Bacteroides | Porphyromonas |
| stercoris | | FBI00082 FBI00097 | xylani solvens | asaccharolytica |
| Bacteroides | Eisenbergiella | Ruminococcaceae sp. | Bifidobacterium | Roseburia hominis |
| thetaiotaomicron | tayi | FBI00233 | adolescentis | |
| Bacteroides | Eubacterium | Ruminococcus bromii | Bifidobacterium | Ruminococcus |
| uniformis | eligens | | longum | bromii |
| Bacteroides | Eubacterium | Ruminococcus faecis | Bifidobacterium | Ruminococcus |
| vulgatus | hallii | | pseudocatenulatum | faecis |
| Barnesiella | Eubacterium | Ruthenibacterium | Bilophila | Sutterella |
| intestinihominis | rectale | lactatiformans | wadsworthia | wadsworthensis |
| Bifidobacterium | Eubacterium | Senegalimassilia | Blautia obeum | |
| bifidum | siraeum | anaerobia | | |
| Bifidobacterium | Eubacterium | Sutterella massiliensis | Blautia wexlerae | |
| catenulatum | ventriosum | | | |
| Bifidobacterium | Eubacterium | Sutterella | Clostridium | |
| dentium | xylanophilum | wadsworthensis | aldenense | |

TABLE 12
Consortia XII

| | | | | |
|---|---|---|---|---|
| Acidaminococcus | Bacteroides | Clostridium | Faecalibacterium | Parasutterella |
| intestini | uniformis | bolteae | prausnitzii | excrementihominis |
| Akkermansia | Bacteroides | Clostridium | Faecalibacterium | Phascolarctobacterium |
| muciniphila | vulgatus | bolteae | prausnitzii | faecium |
| Alistipes | Bacteroides | Clostridium | Fusicatenibacter | Phascolarctobacterium |
| onderdonkii | vulgatus | citroniae | saccharivorans | faecium |
| Alistipes | Bacteroides | Clostridium | Fusicatenibacter | Porphyromonas |
| onderdonkii | xylanisolvens | citroniae | saccharivorans | asaccharolytica |
| Alistipes | Bacteroides | Clostridium | Gordonibacter | Porphyromonas |
| putredinis | xylanisolvens | clostridioforme | pamelaeae | asaccharolytica |
| Alistipes | Bacteroides | Clostridium | Gordonibacter | Roseburia hominis |
| putredinis | xylanisolvens | fessum | pamelaeae | |
| Alistipes | Barnesiella | Clostridium | Holdemanella | Roseburia hominis |
| senegalensis | intestinihominis | fessum | biformis | |
| Alistipes shahii | Bifidobacterium | Clostridium | Holdemanella | Ruminococcaceae sp. |
| | adolescentis | scindens | biformis | FBI00082 FBI00097 |
| Alistipes shahii | Bifidobacterium | Collinsella | Hungatella | Ruminococcaceae sp. |
| | adolescentis | aerofaciens | effluvii | FBI00082 FBI00097 |
| Alistipes sp. | Bifidobacterium | Collinsella | Hungatella | Ruminococcaceae sp. |
| FBI00180 | adolescentis | aerofaciens | effluvii | FBI00233 |
| Alistipes sp. | Bifidobacterium | Coprococcus | Hungatella | Ruminococcus bromii |
| FBI00238 | bifidum | comes | effluvii | |
| Alistipes | Bifidobacterium | Coprococcus | Lachnoclostridium | Ruminococcus bromii |
| timonensis | bifidum | comes | pacaense | |
| Anaerofustis | Bifidobacterium | Coprococcus | Lachnoclostridium | Ruminococcus faecis |
| stercorihominis | catenulatum | eutactus | pacaense | |
| Anaerostipes | Bifidobacterium | Coprococcus | Lachnospiraceae | Ruminococcus faecis |
| hadrus | dentium | eutactus | sp. FBI00033 | |
| Anaerostipes | Bifidobacterium | Dialister | Lachnospiraceae | Ruthenibacterium |
| hadrus | longum | invisus | sp. FBI00071 | lactatiformans |
| Anaerotruncus | Bifidobacterium | Dialister | Lachnospiraceae | Senegalimassilia |
| massiliensis | longum | succinatiphilus | sp. FBI00290 | anaerobia |
| Bacteroides | Bifidobacterium | Dielma | Lactobacillus | Sutterella massiliensis |
| caccae | pseudocatenulatum | fastidiosa | rogosae | |
| Bacteroides | Bifidobacterium | Dorea | Lactobacillus | Sutterella wadsworthensis |
| caccae | pseudocatenulatum | formicigenerans | rogosae | |
| Bacteroides | Bifidobacterium | Dorea | Longicatena | Sutterella wadsworthensis |
| coprocola | pseudocatenulatum | formicigenerans | caecimuris | |
| Bacteroides | Bilophila | Dorea | Megasphaera | Turicibacter sanguinis |
| faecis | wadsworthia | longicatena | massiliensis | |
| Bacteroides | Bilophila | Dorea | Methanobrevibacter | Bacteroides |
| finegoldii | wadsworthia | longicatena | smithii | thetaiotaomicron |
| Bacteroides | Blautia faecis | Eggerthella | Methanobrevibacter | Bacteroides uniformis |
| fragilis | | lenta | smithii | |
| Bacteroides | Blautia faecis | Eggerthella | Monoglobus | Clostridium aldenense |
| kribbi/ | | lenta | pectinilyticus | |
| Bacteroides | | | | |
| koreensis | | | | |
| species cluster | | | | |
| Bacteroides | Blautia | Eggerthella | Monoglobus | Clostridium aldenense |
| kribbi/ | hydrogeno- | lenta | pectinilyticus | |
| Bacteroides | trophica | | | |
| koreensis | | | | |
| species cluster | | | | |
| Bacteroides | Blautia | Eggerthella | Neglecta | Eubacterium ventriosum |
| kribbi/ | massiliensis | lenta | timonensis | |
| Bacteroides | | | | |
| koreensis | | | | |
| species cluster | | | | |
| Bacteroides | Blautia obeum | Eisenbergiella | Neglecta | Eubacterium xylanophilum |
| massiliensis | | tayi | timonensis | |
| Bacteroides | Blautia obeum | Eisenbergiella | Oxalobacter | Paraprevotella clara |
| nordii | | tayi | formigenes | |
| Bacteroides | Blautia wexlerae | Eubacterium | Oxalobacter | Parasutterella |
| ovatus | | eligens | formigenes | excrementihominis |
| Bacteroides | Blautia wexlerae | Eubacterium | Oxalobacter | Bacteroides |
| salyersiae | | eligens | formigenes | thetaiotaomicron |
| Bacteroides | Butyricimonas | Eubacterium | Parabacteroides | Clostridiales sp. FBI00377 |
| stercorirosoris | faecihominis | hallii | distasonis | |
| Bacteroides | Catabacter | Eubacterium | Parabacteroides | Eubacterium siraeum |
| stercoris | hongkongensis | rectale | distasonis | |
| Bacteroides | Clostridiaceae sp. | Eubacterium | Parabacteroides | Parabacteroides merdae |
| stercoris | FBI00191 | rectale | merdae | |

TABLE 13
Consortia XIII

| | | | | |
|---|---|---|---|---|
| Acidaminococcus | Bacteroides | Butyricimonas | Eubacterium | Monoglobus |
| intestini | thetaiotaomicron | faecihominis | eligens | pectinilyticus |
| Akkermansia | Bacteroides | Catabacter | Eubacterium eligens | Neglecta timonensis |
| muciniphila | thetaiotaomicron | hongkongensis | | |
| Alistipes | Bacteroides | Clostridiaceae | Eubacterium hallii | Neglecta timonensis |
| onderdonkii | uniformis | sp. FBI00191 | | |
| Alistipes | Bacteroides | Clostridiales sp. | Eubacterium rectale | Oxalobacter |
| onderdonkii | uniformis | FBI00377 | | formigenes |
| Alistipes | Bacteroides | Clostridium | Eubacterium | Oxalobacter |
| putredinis | vulgatus | aldenense | rectale | formigenes |
| Alistipes | Bacteroides | Clostridium | Eubacterium siraeum | Oxalobacter |
| putredinis | vulgatus | aldenense | | formigenes |
| Alistipes | Bacteroides | Clostridium | Eubacterium | Parabacteroides |
| senegalensis | xylanisolvens | bolteae | ventriosum | distasonis |
| Alistipes shahii | Bacteroides | Clostridium | Eubacterium | Parabacteroides |
| | xylanisolvens | bolteae | xylanophilum | distasonis |
| Alistipes shahii | Bacteroides | Clostridium | Faecalibacterium | Parabacteroides |
| | xylanisolvens | citroniae | prausnitzii | merdae |
| Alistipes sp. | Barnesiella | Clostridium | Faecalibacterium | Parabacteroides |
| FBI00180 | intestinihominis | citroniae | prausnitzii | merdae |
| Alistipes sp. | Bifidobacterium | Clostridium | Fusicatenibacter | Paraprevotella clara |
| FBI00238 | adolescentis | clostridioforme | saccharivorans | |
| Alistipes | Bifidobacterium | Clostridium | Fusicatenibacter | Parasutterella |
| timonensis | adolescentis | fessum | saccharivorans | excrementihominis |
| Anaerofustis | Bifidobacterium | Clostridium | Gordonibacter | Parasutterella |
| stercorihominis | adolescentis | scindens | pamelaeae | excrementihominis |
| Anaerostipes | Bifidobacterium | Collinsella | Gordonibacter | Phascolarctobacterium |
| hadrus | bifidum | aerofaciens | pamelaeae | faecium |
| Anaerostipes | Bifidobacterium | Collinsella | Holdemanella | Porphyromonas |
| hadrus | catenulatum | aerofaciens | biformis | asaccharolytica |
| Anaerotruncus | Bifidobacterium | Coprococcus | Holdemanella | Porphyromonas |
| massiliensis | dentium | comes | biformis | asaccharolytica |
| Bacteroides | Bifidobacterium | Coprococcus | Hungatella effluvii | Roseburia hominis |
| caccae | longum | comes | | |
| Bacteroides | Bifidobacterium | Coprococcus | Hungatella effluvii | Roseburia hominis |
| caccae | longum | eutactus | | |
| Bacteroides | Bifidobacterium | Dialister invisus | Hungatella effluvii | Ruminococcaceae sp. |
| coprocola | pseudocatenulatum | | | FBI00082 FBI00097 |
| Bacteroides | Bifidobacterium | Dialister | Lachnoclostridium | Ruminococcaceae sp. |
| faecis | pseudocatenulatum | succinatiphilus | pacaense | FBI00233 |
| Bacteroides | Bifidobacterium | Dielma fastidiosa | Lachnoclostridium | Ruminococcus bromii |
| finegoldii | pseudocatenulatum | | pacaense | |
| Bacteroides | Bilophila | Dorea | Lachnospiraceae sp. | Ruminococcus |
| fragilis | wadsworthia | formicigenerans | FBI00033 | bromii |
| Bacteroides | Bilophila | Dorea | Lachnospiraceae sp. | Ruminococcus faecis |
| kribbi/ | wadsworthia | formicigenerans | FBI00071 | |
| Bacteroides | | | | |
| koreensis species | | | | |
| cluster | | | | |
| Bacteroides | Blautia faecis | Dorea | Lachnospiraceae sp. | Ruminococcus |
| kribbi/ | | longicatena | FBI00290 | faecis |
| Bacteroides | | | | |
| koreensis | | | | |
| species cluster | | | | |
| Bacteroides | Blautia faecis | Dorea | Lactobacillus | Ruthenibacterium |
| massiliensis | | longicatena | rogosae | lactatiformans |
| Bacteroides | Blautia | Eggerthella lenta | Lactobacillus | Senegalimassilia |
| nordii | hydrogenotrophica | | rogosae | anaerobia |
| Bacteroides | Blautia massiliensis | Eggerthella | Longicatena | Sutterella massiliensis |
| ovatus | | lenta | caecimuris | |
| Bacteroides | Blautia obeum | Eggerthella | Megasphaera | Sutterella |
| salyersiae | | lenta | massiliensis | wadsworthensis |
| Bacteroides | Blautia obeum | Eggerthella | Methanobrevibacter | Sutterella |
| stercorirosoris | | lenta | smithii | wadsworthensis |
| Bacteroides | Blautia wexlerae | Eisenbergiella | Methanobrevibacter | Turicibacter sanguinis |
| stercoris | | tayi | smithii | |
| Bacteroides | Blautia wexlerae | Eisenbergiella | Monoglobus | |
| stercoris | | tayi | pectinilyticus | |

TABLE 14
Consortia XIV

| | | | | |
|---|---|---|---|---|
| Acidaminococcus | Bacteroides | Clostridium | Eubacterium siraeum | Parasutterella |
| intestini | uniformis | citroniae | | excrementihominis |
| Akkermansia | Bacteroides | Clostridium | Eubacterium | Parasutterella |
| muciniphila | uniformis | citroniae | ruminantium | excrementihominis |
| Alistipes | Bacteroides | Clostridium | Eubacterium ventriosum | Phascolarctobacterium |
| finegoldii | vulgatus | clostridioforme | | faecium |
| Alistipes | Bacteroides | Clostridium | Eubacterium | Phascolarctobacterium |
| onderdonkii | vulgatus | scindens | xylanophilum | faecium |
| Alistipes | Bacteroides | Clostridium | Faecalibacterium | Phocea massiliensis |
| onderdonkii | xylanisolvens | swellfunianum | prausnitzii | |
| Alistipes | Bacteroides | Clostridium | Faecalibacterium | Phocea massiliensis |
| putredinis | xylanisolvens | symbiosum | prausnitzii | |
| Alistipes | Barnesiella | Clostridium | Fusicatenibacter | Porphyromonas |
| putredinis | intestinihominis | symbiosum | saccharivorans | asaccharolytica |
| Alistipes | Bifidobacterium | Collinsella | Fusicatenibacter | Porphyromonas |
| senegalensis | adolescentis | aerofaciens | saccharivorans | asaccharolytica |
| Alistipes | Bifidobacterium | Collinsella | Gordonibacter | Roseburia hominis |
| senegalensis | adolescentis | aerofaciens | pamelaeae | |
| Alistipes shahii | Bifidobacterium | Coprococcus | Gordonibacter | Roseburia hominis |
| | bifidum | comes | pamelaeae | |
| Alistipes shahii | Bifidobacterium | Coprococcus | Holdemanella biformis | Ruminococcus bromii |
| | bifidum | comes | | |
| Alistipes | Bifidobacterium | Coprococcus | Holdemanella biformis | Ruminococcus bromii |
| timonensis | catenulatum | eutactus | | |
| Anaerofustis | Bifidobacterium | Coprococcus | Hungatella effluvii | Ruminococcus faecis |
| stercorihominis | dentium | eutactus | | |
| Anaerostipes | Bifidobacterium | Desulfovibrio | Hungatella hathewayi | Ruminococcus faecis |
| hadrus | faecale | desulfuricans | | |
| Anaerostipes | Bifidobacterium | Desulfovibrio | Hungatella hathewayi | Ruthenibacterium |
| hadrus | longum | desulfuricans | | lactatiformans |
| Anaerotruncus | Bifidobacterium | Dialister | Hydrogenoanaerobacterium | Senegalimassilia |
| colihominis | longum | invisus | saccharovorans | anaerobia |
| Bacteroides | Bifidobacterium | Dialister | Lachnoclostridium | Sutterella massiliensis |
| caccae | pseudocatenulatum | succinatiphilus | pacaense | |
| Bacteroides | Bifidobacterium | Dielma | Lachnoclostridium | Sutterella wadsworthensis |
| caccae | pseudocatenulatum | fastidiosa | pacaense | |
| Bacteroides | Bifidobacterium | Dorea | Lachnospira | Sutterella wadsworthensis |
| coprocola | pseudocatenulatum | formicigenerans | pectinoschiza | |
| Bacteroides | Blautia faecis | Dorea | Lachnospira | Turicibacter sanguinis |
| faecis | | formicigenerans | pectinoschiza | |
| Bacteroides | Blautia faecis | Dorea | Longicatena caecimuris | Bacteroides stercoris |
| finegoldii | | longicatena | | |
| Bacteroides | Blautia | Dorea | Megasphaera | Bacteroides stercoris |
| fragilis | hydrogenotrophica | longicatena | massiliensis | |
| Bacteroides | Blautia luti | Eggerthella | Methanobrevibacter smithii | Bacteroides |
| koreensis | | lenta | | thetaiotaomicron |
| Bacteroides | Blautia obeum | Eggerthella | Methanobrevibacter smithii | Bacteroides |
| koreensis | | lenta | | thetaiotaomicron |
| Bacteroides | Blautia obeum | Eggerthella | Monoglobus | Clostridium aldenense |
| kribbi | | lenta | pectinilyticus | |
| Bacteroides | Blautia wexlerae | Eggerthella | Monoglobus | Clostridium aldenense |
| kribbi | | lenta | pectinilyticus | |
| Bacteroides | Blautia wexlerae | Eisenbergiella | Neglecta timonensis | Clostridium bolteae |
| massiliensis | | tayi | | |
| Bacteroides | Butyricimonas | Eisenbergiella | Oxalobacter formigenes | Clostridium bolteae |
| nordii | faecihominis | tayi | | |
| Bacteroides | Catabacter | Emergencia | Oxalobacter formigenes | Parabacteroides merdae |
| oleiciplenus | hongkongensis | timonensis | | |
| Bacteroides | Citrobacter | Eubacterium | Oxalobacter formigenes | Parabacteroides merdae |
| ovatus | freundii | eligens | | |
| Bacteroides | Clostridiaceae | Eubacterium | Parabacteroides | Paraprevotella clara |
| salyersiae | sp. | eligens | distasonis | |
| Eubacterium | Eubacterium | Eubacterium | Parabacteroides | Eubacterium |
| rectale | rectale | hallii | distasonis | oxidoreducens |

TABLE 15
Consortia XV

| | | | | |
|---|---|---|---|---|
| Acidaminococcus | Bacteroides | Butyricimonas | Eubacterium | Monoglobus |
| intestini | thetaiotaomicron | faecihominis | eligens | pectinilyticus |
| Akkermansia | Bacteroides | Catabacter | Eubacterium eligens | Neglecta timonensis |
| muciniphila | thetaiotaomicron | hongkongensis | | |
| Alistipes | Bacteroides | Clostridiaceae | Eubacterium hallii | Neglecta timonensis |
| onderdonkii | uniformis | sp. FBI00191 | | |
| Alistipes | Bacteroides | Clostridiales sp. | Eubacterium rectale | Oxalobacter |
| onderdonkii | uniformis | FBI00377 | | formigenes |
| Alistipes | Bacteroides | Clostridium | Eubacterium | Oxalobacter |
| putredinis | vulgatus | aldenense | rectale | formigenes |
| Alistipes | Bacteroides | Clostridium | Eubacterium siraeum | Oxalobacter |
| putredinis | vulgatus | aldenense | | formigenes |
| Alistipes | Bacteroides | Clostridium | Eubacterium | Parabacteroides |
| senegalensis | xylanisolvens | bolteae | ventriosum | distasonis |
| Alistipes shahii | Bacteroides | Clostridium | Eubacterium | Parabacteroides |
| | xylanisolvens | bolteae | xylanophilum | distasonis |
| Alistipes shahii | Bacteroides | Clostridium | Faecalibacterium | Parabacteroides |
| | xylanisolvens | citroniae | prausnitzii | merdae |
| Alistipes sp. | Barnesiella | Clostridium | Faecalibacterium | Parabacteroides |
| FBI00180 | intestinihominis | citroniae | prausnitzii | merdae |
| Alistipes sp. | Bifidobacterium | Clostridium | Fusicatenibacter | Paraprevotella clara |
| FBI00238 | adolescentis | clostridioforme | saccharivorans | |
| Alistipes | Bifidobacterium | Clostridium | Fusicatenibacter | Parasutterella |
| timonensis | adolescentis | fessum | saccharivorans | excrementihominis |
| Anaerofustis | Bifidobacterium | Clostridium | Gordonibacter | Parasutterella |
| stercorihominis | adolescentis | scindens | pamelaeae | excrementihominis |
| Anaerostipes | Bifidobacterium | Collinsella | Gordonibacter | Phascolarctobacterium |
| hadrus | bifidum | aerofaciens | pamelaeae | faecium |
| Anaerostipes | Bifidobacterium | Collinsella | Holdemanella | Porphyromonas |
| hadrus | catenulatum | aerofaciens | biformis | asaccharolytica |
| Anaerotruncus | Bifidobacterium | Coprococcus | Holdemanella | Porphyromonas |
| massiliensis | dentium | comes | biformis | asaccharolytica |
| Bacteroides | Bifidobacterium | Coprococcus | Hungatella effluvii | Roseburia hominis |
| caccae | longum | comes | | |
| Bacteroides | Bifidobacterium | Coprococcus | Hungatella effluvii | Roseburia hominis |
| caccae | longum | eutactus | | |
| Bacteroides | Bifidobacterium | Dialister invisus | Hungatella effluvii | Ruminococcaceae sp. |
| coprocola | pseudocatenulatum | | | FBI00082 FBI00097 |
| Bacteroides | Bifidobacterium | Dialister | Lachnoclostridium | Ruminococcaceae sp. |
| faecis | pseudocatenulatum | succinatiphilus | pacaense | FBI00233 |
| Bacteroides | Bifidobacterium | Dielma fastidiosa | Lachnoclostridium | Ruminococcus bromii |
| finegoldii | pseudocatenulatum | | pacaense | |
| Bacteroides | Bilophila | Dorea | Lachnospiraceae sp. | Ruminococcus |
| fragilis | wadsworthia | formicigenerans | FBI00033 | bromii |
| Bacteroides | Bilophila | Dorea | Lachnospiraceae sp. | Ruminococcus faecis |
| kribbi/ | wadsworthia | formicigenerans | FBI00071 | |
| Bacteroides | | | | |
| koreensis species | | | | |
| cluster | | | | |
| Bacteroides | Blautia faecis | Dorea | Lachnospiraceae sp. | Ruminococcus |
| kribbi/ | | longicatena | FBI00290 | faecis |
| Bacteroides | | | | |
| koreensis | | | | |
| species cluster | | | | |
| Bacteroides | Blautia faecis | Dorea | Lactobacillus | Ruthenibacterium |
| massiliensis | | longicatena | rogosae | lactatiformans |
| Bacteroides | Blautia | Eggerthella lenta | Lactobacillus | Senegalimassilia |
| nordii | hydrogenotrophica | | rogosae | anaerobia |
| Bacteroides | Blautia massiliensis | Eggerthella | Longicatena | Sutterella massiliensis |
| ovatus | | lenta | caecimuris | |
| Bacteroides | Blautia obeum | Eggerthella | Megasphaera | Sutterella |
| salyersiae | | lenta | massiliensis | wadsworthensis |
| Bacteroides | Blautia obeum | Eggerthella | Methanobrevibacter | Sutterella |
| stercorirosoris | | lenta | smithii | wadsworthensis |
| Bacteroides | Blautia wexlerae | Eisenbergiella | Methanobrevibacter | Turicibacter sanguinis |
| stercoris | | tayi | smithii | |
| Bacteroides | Blautia wexlerae | Eisenbergiella | Monoglobus | |
| stercoris | | tayi | pectinilyticus | |

TABLE 16
Consortia XVI

| | | | | |
|---|---|---|---|---|
| Acidaminococcus | Bacteroides | Clostridium | Eubacterium | Parasutterella |
| intestini | uniformis | citroniae | ventriosum | excrementihominis |
| Akkermansia | Bacteroides | Clostridium | Eubacterium | Phascolarctobacterium |
| muciniphila | uniformis | clostridioforme | siraeum | faecium |
| Alistipes | Bacteroides | Clostridium | Eubacterium | Phascolarctobacterium |
| finegoldii | vulgatus | scindens | xylanophilum | faecium |
| Alistipes | Bacteroides | Clostridium | Faecalibacterium | Phocea massiliensis |
| onderdonkii | vulgatus | swellfunianum | prausnitzii | |
| Alistipes | Bacteroides | Clostridium | Faecalibacterium | Phocea massiliensis |
| onderdonkii | xylanisolvens | symbiosum | prausnitzii | |
| Alistipes | Bacteroides | Clostridium | Fusicatenibacter | Porphyromonas |
| putredinis | xylanisolvens | symbiosum | saccharivorans | asaccharolytica |
| Alistipes | Barnesiella | Collinsella | Fusicatenibacter | Porphyromonas |
| putredinis | intestinihominis | aerofaciens | saccharivorans | asaccharolytica |
| Alistipes | Bifidobacterium | Collinsella | Gordonibacter | Roseburia hominis |
| senegalensis | adolescentis | aerofaciens | pamelaeae | |
| Alistipes | Bifidobacterium | Coprococcus | Gordonibacter | Roseburia hominis |
| senegalensis | adolescentis | comes | pamelaeae | |
| Alistipes shahii | Bifidobacterium | Coprococcus | Holdemanella | Ruminococcus bromii |
| | bifidum | comes | biformis | |
| Alistipes | Bifidobacterium | Coprococcus | Holdemanella | Ruminococcus |
| shahii | bifidum | eutactus | biformis | bromii |
| Alistipes | Bifidobacterium | Coprococcus | Hungatella | Ruminococcus faecis |
| timonensis | catenulatum | eutactus | effluvii | |
| Anaerofustis | Bifidobacterium | Desulfovibrio | Hungatella | Ruminococcus faecis |
| stercorihominis | dentium | desulfuricans | hathewayi | |
| Anaerostipes | Bifidobacterium | Desulfovibrio | Hungatella | Ruthenibacterium |
| hadrus | faecale | desulfuricans | hathewayi | lactatiformans |
| Anaerostipes | Bifidobacterium | Dialister invisus | Hydrogenoanaero- | Senegalimassilia |
| hadrus | longum | | bacterium | anaerobia |
| | | | saccharovorans | |
| Anaerotruncus | Bifidobacterium | Dialister | Lachnoclostridium | Sutterella massiliensis |
| colihominis | longum | succinatiphilus | pacaense | |
| Bacteroides | Bifidobacterium | Dielma fastidiosa | Lachnoclostridium | Sutterella |
| caccae | pseudocatenulatum | | pacaense | wadsworthensis |
| Bacteroides | Bifidobacterium | Dorea | Lachnospira | Sutterella |
| caccae | pseudocatenulatum | formicigenerans | pectinoschiza | wadsworthensis |
| Bacteroides | Bifidobacterium | Dorea | Lachnospira | Turicibacter sanguinis |
| coprocola | pseudocatenulatum | formicigenerans | pectinoschiza | |
| Bacteroides | Blautia faecis | Dorea | Longicatena | Bacteroides stercoris |
| faecis | | longicatena | caecimuris | |
| Bacteroides | Blautia faecis | Dorea | Megasphaera | Bacteroides stercoris |
| finegoldii | | longicatena | massiliensis | |
| Bacteroides | Blautia | Eggerthella lenta | Methanobrevibacter | Bacteroides |
| fragilis | hydrogenotrophica | | smithii | thetaiotaomicron |
| Bacteroides | Blautia luti | Eggerthella | Methanobrevibacter | Bacteroides |
| koreensis | | lenta | smithii | thetaiotaomicron |
| Bacteroides | Blautia obeum | Eggerthella | Monoglobus | Clostridium |
| koreensis | | lenta | pectinilyticus | aldenense |
| Bacteroides | Blautia obeum | Eggerthella | Monoglobus | Clostridium bolteae |
| kribbi | | lenta | pectinilyticus | |
| Bacteroides | Blautia wexlerae | Eisenbergiella | Neglecta | Clostridium bolteae |
| kribbi | | tayi | timonensis | |
| Bacteroides | Blautia wexlerae | Eisenbergiella | Oxalobacter | Clostridium citroniae |
| massiliensis | | tayi | formigenes | |
| Bacteroides | Butyricimonas | Emergencia | Oxalobacter | Eubacterium |
| nordii | faecihominis | timonensis | formigenes | oxidoreducens |
| Bacteroides | Catabacter | Eubacterium | Oxalobacter | Eubacterium rectale |
| oleiciplenus | hongkongensis | eligens | formigenes | |
| Bacteroides | Clostridiaceae sp. | Eubacterium | Parabacteroides | Eubacterium rectale |
| ovatus | | eligens | distasonis | |
| Bacteroides | Clostridium | Eubacterium | Parabacteroides | Eubacterium |
| salyersiae | aldenense | hallii | distasonis | ruminantium |
| Parasutterella | Paraprevotella | Parabacteroides | Parabacteroides | |
| excrementihominis | clara | merdae | merdae | |

TABLE 17
Consortia XVII

| | | | | |
|---|---|---|---|---|
| Acidaminococcus | Bacteroides | Clostridium | Faecalicatena | Roseburia |
| intestini | uniformis | bolteae | contorta | hominis |
| Acutalibacter | Bacteroides | Clostridium | Fusicatenibacter | Roseburia |
| timonensis | vulgatus | citroniae | saccharivorans | hominis |
| Akkermansia | Bacteroides | Clostridium | Fusicatenibacter | Ruminococcaceae |
| muciniphila | vulgatus | citroniae | saccharivorans | sp. FBI00097 |
| Alistipes | Bacteroides | Clostridium | Gordonibacter | Ruminococcaceae |
| onderdonkii | xylanisolvens | clostridioforme | pamelaeae | sp. FBI00097 |
| Alistipes | Bacteroides | Clostridium | Gordonibacter | Ruminococcaceae |
| onderdonkii | xylanisolvens | fessum | pamelaeae | sp. FBI00233 |
| Alistipes | Bacteroides | Clostridium | Holdemanella | Ruminococcus |
| putredinis | xylanisolvens | fessum | biformis | bromii |
| Alistipes | Barnesiella | Clostridium | Holdemanella | Ruminococcus |
| putredinis | intestinihominis | scindens | biformis | bromii |
| Alistipes | Bifidobacterium | Collinsella | Hungatella | Ruminococcus |
| Senegalensis | adolescentis | aerofaciens | effluvii | faecis |
| Alistipes shahii | Bifidobacterium | Collinsella | Hungatella | Ruminococcus |
| | adolescentis | aerofaciens | effluvii | faecis |
| Alistipes sp. | Bifidobacterium | Coprococcus | Hungatella | Ruthenibacterium |
| FBI00180 | adolescentis | comes | effluvii | lactatiformans |
| Alistipes sp. | Bifidobacterium | Coprococcus | Lachnoclostridium | Senegalimassilia |
| FBI00238 | bifidum | comes | pacaense | anaerobia |
| Alistipes | Bifidobacterium | Coprococcus | Lachnoclostridium | Sutterella |
| timonensis | bifidum | eutactus | pacaense | massiliensis |
| Anaerofustis | Bifidobacterium | Coprococcus | Lachnospiraceae | Sutterella |
| stercorihominis | catenulatum | eutactus | sp. FBI00033 | wadsworthensis |
| Anaerostipes | Bifidobacterium | Dialister invisus | Lachnospiraceae | Sutterella |
| hadrus | dentium | | sp. FBI00071 | wadsworthensis |
| Anaerostipes | Bifidobacterium | Dialister | Lachnospiraceae | Turicibacter |
| hadrus | longum | succinatiphilus | sp. FBI00290 | sanguinis |
| Anaerotruncus | Bifidobacterium | Dielma | Lactobacillus | Bacteroides |
| massiliensis | longum | fastidiosa | rogosae | stercoris |
| Bacteroides caccae | Bifidobacterium | Dorea | Lactobacillus | Bacteroides |
| | pseudocatenulatum | formicigenerans | rogosae | stercoris |
| Bacteroides caccae | Bifidobacterium | Dorea | Lactonifactor | Bacteroides |
| | pseudocatenulatum | formicigenerans | longoviformis | thetaiotaomicron |
| Bacteroides | Bilophila | Dorea | Longicatena | Bacteroides |
| coprocola | wadsworthia | longicatena | caecimuris | thetaiotaomicron |
| Bacteroides faecis | Bilophila | Dorea | Megasphaera | Bacteroides |
| | wadsworthia | longicatena | massiliensis | uniformis |
| Bacteroides | Blautia faecis | Eggerthella | Monoglobus | Citrobacter |
| finegoldii | | lenta | pectinilyticus | portucalensis |
| Bacteroides | Blautia faecis | Eggerthella | Monoglobus | Clostridiaceae |
| fragilis | | lenta | pectinilyticus | sp. FBI00191 |
| Bacteroides kribbi/ | Blautia | Eisenbergiella | Oxalob acter | Clostridium |
| Bacteroides | hydrogenotrophica | tayi | formigenes | aldenense |
| koreensis species | | | | |
| cluster | | | | |
| Bacteroides kribbi/ | Blautia | Eisenbergiella | Oxalobacter | Clostridium |
| Bacteroides | massiliensis | tayi | formigenes | aldenense |
| koreensis species | | | | |
| cluster | | | | |
| Bacteroides kribbi/ | Blautia obeum | Emergencia | Oxalobacter | Clostridium |
| Bacteroides | | timonensis | formigenes | bolteae |
| koreensis species | | | | |
| cluster | | | | |
| Bacteroides | Blautia obeum | Eubacterium | Parabacteroides | Eubacterium |
| massiliensis | | eligens | distasonis | siraeum |
| Bacteroides nordii | Blautia wexlerae | Eubacterium | Parabacteroides | Eubacterium |
| | | eligens | merdae | ventriosum |
| Bacteroides ovatus | Blautia wexlerae | Eubacterium | Parabacteroides | Eubacterium |
| | | hallii | merdae | xylanophilum |
| Bacteroides | Butyricimonas | Eubacterium | Paraprevotella | Faecalibacterium |
| salyersiae | faecihominis | rectale | clara | prausnitzii |
| Bacteroides | Catabacter | Eubacterium | Parasutterella | Faecalibacterium |
| stercorirosoris | hongkongensis | rectale | excrementihom | prausnitzii |
| | | | inis | |
| Phascolarcto- | Phascolarcto- | Porphyromonas | Parasutterella | Porphyromonas |
| bacterium | bacterium faecium | asaccharolytica | excrementi- | asaccharolytica |
| faecium | | | hominis | |

TABLE 18
Consortia XVIII

| | | | | |
|---|---|---|---|---|
| Bacteroides | Bifidobacterium | Clostridium | Eggerthella | Parabacteroides |
| caccae | longum | scindens | lenta | merdae |
| Bacteroides | Bifidobacterium | Clostridium | Eggerthella | Ruminococcus |
| salyersiae | pseudocatenulatum | symbiosum | lenta | bromii |
| Bacteroides | Bifidobacterium | Collinsella | Faecalibacterium | Ruminococcus |
| thetaiotaomicron | pseudocatenulatum | aerofaciens | prausnitzii | bromii |
| Bacteroides | Bifidobacterium | Desulfovibrio | Neglecta | |
| thetaiotaomicron | pseudocatenulatum | desulfuricans | timonensis | |
| Bacteroides | Clostridium | Dorea | Oxalobacter | |
| vulgatus | amygdalinum | longicatena | formigenes | |
| Bifidobacterium | Clostridium | Eggerthella | Oxalobacter | |
| dentium | citroniae | lenta | formigenes | |
| Bifidobacterium | Clostridium | Eggerthella | Oxalobacter | |
| longum | citroniae | lenta | formigenes | |

TABLE 19
Consortia XIX

| | | | | |
|---|---|---|---|---|
| Acidaminococcus | Bacteroides stercoris | Blautia wexlerae | Eisenbergiella tayi | Monoglobus |
| intestini | | | | pectinilyticus |
| Acutalibacter | Bacteroides stercoris | Blautia wexlerae | Eisenbergiella tayi | Monoglobus |
| timonensis | | | | pectinilyticus |
| Akkermansia | Bacteroides | Butyricimonas | Emergencia | Parabacteroides |
| muciniphila | thetaiotaomicron | faecihominis | timonensis | distasonis |
| Alistipes | Bacteroides | Catabacter | Eubacterium | Oxalobacter |
| onderdonkii | thetaiotaomicron | hongkongensis | eligens | formigenes |
| Alistipes | Bacteroides uniformis | Clostridiaceae sp. | Eubacterium | Oxalobacter |
| onderdonkii | | FBI00191 | eligens | formigenes |
| Alistipes | Bacteroides uniformis | Clostridium | Eubacterium hallii | Oxalobacter |
| putredinis | | aldenense | | formigenes |
| Alistipes | Bacteroides vulgatus | Clostridium | Eubacterium rectale | Parabacteroides |
| putredinis | | aldenense | | distasonis |
| Alistipes | Bacteroides vulgatus | Clostridium | Eubacterium rectale | Parabacteroides |
| senegalensis | | bolteae | | merdae |
| Alistipes shahii | Bacteroides | Clostridium | Eubacterium | Parabacteroides |
| | xylanisolvens | bolteae | siraeum | merdae |
| Alistipes shahii | Bacteroides | Clostridium | Eubacterium | Paraprevotella clara |
| | xylanisolvens | citroniae | ventriosum | |
| Alistipes sp. | Bacteroides | Clostridium | Eubacterium | Parasutterella |
| FBI00180 | xylanisolvens | citroniae | xylanophilum | excrementihominis |
| Alistipes sp. | Bamesiella | Clostridium | Faecalibacterium | Parasutterella |
| FBI00238 | intestinihominis | clostridioforme | prausnitzii | excrementihominis |
| Alistipes | Bifidobacterium | Clostridium | Fusicatenibacter | Phascolarctobacterium |
| timonensis | adolescentis | fessum | saccharivorans | faecium |
| Anaerofustis | Bifidobacterium | Clostridium | Fusicatenibacter | Porphyromonas |
| stercorihominis | adolescentis | scindens | saccharivorans | asaccharolytica |
| Anaerostipes | Bifidobacterium | Collinsella | Gordonibacter | Roseburia hominis |
| hadrus | bifidum | aerofaciens | pamelaeae | |
| Anaerostipes | Bifidobacterium | Collinsella | Gordonibacter | Roseburia hominis |
| hadrus | catenulatum | aerofaciens | pamelaeae | |
| Anaerotruncus | Bifidobacterium | Coprococcus | Holdemanella | Ruminococcaceae |
| massiliensis | dentium | comes | biformis | sp. FBI00097 |
| Bacteroides | Bifidobacterium | Coprococcus | Hungatella effluvii | Ruminococcaceae |
| caccae | longum | comes | | sp. FBI00233 |
| Bacteroides | Bifidobacterium | Coprococcus | Hungatella effluvii | Ruminococcus |
| caccae | longum | eutactus | | bromii |
| Bacteroides | Bifidobacterium | Dialister invisus | Hungatella | Ruminococcus |
| coprocola | pseudocatenulatum | | hathewayi | bromii |
| Bacteroides | Bifidobacterium | Dialister | Lachnoclostridium | Ruminococcus |
| faecis | pseudocatenulatum | succinatiphilus | pacaense | faecis |
| Bacteroides | Bifidobacterium | Dielma fastidiosa | Lachnoclostridium | Ruminococcus |
| finegoldii | pseudocatenulatum | | pacaense | faecis |
| Bacteroides | Bifidobacterium | Dorea | Lachnospiraceae | Ruthenibacterium |
| fragilis | adolescentis | formicigenerans | sp. FBI00033 | lactatiformans |
| Bacteroides | Bilophila wadsworthia | Dorea | Lachnospiraceae | Senegalimassilia |
| kribbi | | formicigenerans | sp. FBI00071 | anaerobia |
| Bacteroides | Bilophila wadsworthia | Dorea | Lachnospiraceae | Sutterella |
| kribbi | | longicatena | sp. FBI00290 | massiliensis |
| Bacteroides | Blautia faecis | Dorea | Lactobacillus | Sutterella |
| massiliensis | | longicatena | rogosae | wadsworthensis |
| Bacteroides | Blautia | Eggerthella lenta | Lactobacillus | Sutterella |
| nordii | hydrogenotrophica | | rogosae | wadsworthensis |
| Bacteroides | Blautia massiliensis | Eggerthella lenta | Longicatena | Turicibacter |
| ovatus | | | caecimuris | sanguinis |
| Bacteroides | Blautia obeum | Eggerthella lenta | Megasphaera | |
| salyersiae | | | massiliensis | |
| Bacteroides | Blautia obeum | Eggerthella lenta | Methanobrevibacter | |
| stercorirosoris | | | smithii | |

In certain embodiments, the Consortia comprises the microbiota listed in Table 1. In certain embodiments, the Consortia comprises the microbiota listed in Table 2. In certain embodiments, the Consortia comprises the microbiota listed in Table 3. In certain embodiments, the Consortia comprises the microbiota listed in Table 4. In certain embodiments, the Consortia comprises the microbiota listed in Table 5. In certain embodiments, the Consortia comprises the microbiota listed in Table 6. In certain embodiments, the Consortia comprises the microbiota listed in Table 7. In certain embodiments, the Consortia comprises the microbiota listed in Table 8. In certain embodiments, the Consortia comprises the microbiota listed in Table 9. In certain embodiments, the Consortia comprises the microbiota listed in Table 10. In certain embodiments, the Consortia comprises the microbiota listed in Table 11. In certain embodiments, the Consortia comprises the microbiota listed in Table 12. In certain embodiments, the Consortia comprises the microbiota listed in Table 13. In certain embodiments, the Consortia comprises the microbiota listed in Table 14. In certain embodiments, the Consortia comprises the microbiota listed in Table 15. In certain embodiments, the Consortia comprises the microbiota listed in Table 16. In certain embodiments, the Consortia comprises the microbiota listed in Table 17. In certain embodiments, the Consortia comprises the microbiota listed in Table 18. In certain embodiments, the Consortia comprises the microbiota listed in Table 19.

In certain embodiments, the Consortia comprises the microbiota that are at least 90% or at least 95% identical to those listed in Table 1. In certain embodiments, the Consortia comprises the microbiota that are at least 90% or at least 95% identical to those listed in Table 2. In certain embodiments, the Consortia comprises the microbiota that are at least 90% or at least 95% identical to those listed in Table 3. In certain embodiments, the Consortia comprises the microbiota that are at least 90% or at least 95% identical to those listed in Table 4. In certain embodiments, the Consortia comprises the microbiota that are at least 90% or at least 95% identical to those listed in Table 5. In certain embodiments, the Consortia comprises the microbiota that are at least 90% or at least 95% identical to those listed in Table 6. In certain embodiments, the Consortia comprises the microbiota that are at least 90% or at least 95% identical to those listed in Table 7. In certain embodiments, the Consortia comprises the microbiota that are at least 90% or at least 95% identical to those listed in Table 8. In certain embodiments, the Consortia comprises the microbiota that are at least 90% or at least 95% identical to those listed in Table 9. In certain embodiments, the Consortia comprises the microbiota that are at least 90% or at least 95% identical to those listed in Table 10. In certain embodiments, the Consortia comprises the microbiota that are at least 90% or at least 95% identical to those listed in Table 11. In certain embodiments, the Consortia comprises the microbiota that are at least 90% or at least 95% identical to those listed in Table 12. In certain embodiments, the Consortia comprises the microbiota that are at least 90% or at least 95% identical to those listed in Table 13. In certain embodiments, the Consortia comprises the microbiota that are at least 90% or at least 95% identical to those that are at least 90% or at least 95% identical to those listed in Table 14. In certain embodiments, the Consortia comprises the microbiota that are at least 90% or at least 95% identical to those listed in Table 15. In certain embodiments, the Consortia comprises the microbiota that are at least 90% or at least 95% identical to those listed in Table 16. In certain embodiments, the Consortia comprises the microbiota that are at least 90% or at least 95% identical to those listed in Table 17. In certain embodiments, the Consortia comprises the microbiota that are at least 90% or at least 95% identical to those listed in Table 18. In certain embodiments, the Consortia comprises the microbiota that are at least 90% or at least 95% identical to those listed in Table 19.

In certain embodiments, a microbial consortium described herein comprises a microbial strain having a relative abundance of approximately 90%, 80%, 70%, 60%, 50%, 40%, 30%, 20%, 10%, 5%, 1%, 0.1%, 0.01%, 0.001%, 0.0001%, 0.00001%, or 0.000001% of the total microbial consortium. In certain embodiments, the relative abundance of a microbial strain is determined by metagenomic sequencing and calculated as the percentage of reads that are classified as an identified microbial strain, divided by the genome size. In certain embodiments, the relative abundance of a microbial strain of the present disclosure is determined by metagenomic shotgun sequencing.

In certain embodiments, the Consortia comprises the microbiota that are at least 90% or at least 95% identical to those listed in Table 22. Table 22 is provided below:

TABLE 22
FB-001 Drug Substances

| Drug Substance (DS) | | |
|---|---|---|
| (aka CoCulture, CoC) | Strain ID | Strain Species |
| DSI | FBI00001 | Clostridium citroniae |
| | FBI00002 | Bacteroides salyersiae |
| | FBI00010 | Blautia obeum |
| | FBI00013 | Parabacteroides merdae |
| | FBI00029 | Parabacteroides distasonis |
| | FBI00032 | Anaero stipes hadrus |
| | FBI00033 | Lachnospiraceae sp. FBI00033 |
| | FBI00034 | Eubacterium eligens |
| | FBI00043 | Bifidobacterium dentium |
| | FBI00044 | Blautia wexlerae |
| | FBI00048 | Fusicatenibacter saccharivorans |
| | FBI00050 | Bacteroides nordii |
| | FBI00051 | Dorea formicigenerans |
| | FBI00057 | Dorea longicatena |
| | FBI00059 | Bacteroides stercorirosoris |
| | FBI00060 | Bifidobacterium longum |
| | FBI00070 | Bacteroides kribbi |
| | FBI00071 | Lachnospiraceae sp. FBI00071 |
| | FBI00076 | Bacteroides thetaiotaomicron |
| | FBI00079 | Clostridium clostridioforme |
| | FBI00087 | Clostridium scindens |
| | FBI00093 | Roseburia hominis |
| | FBI00102 | Clostridium fessum |
| | FBI00109 | Coprococcus comes |
| | FBI00117 | Blautia faecis |
| | FBI00120 | Hungatella hathewayi |
| | FBI00125 | Bacteroides stercoris |
| | FBI00127 | Collinsella aerofaciens |
| | FBI00128 | Hungatella effluvii |
| | FBI00145 | Bifidobacterium adolescentis |
| | FBI00162 | Bifidobacterium catenulatum |
| | FBI00174 | Lactobacillus rogosae |
| | FBI00184 | Bacteroides faecis |
| | FBI00190 | Bacteroides finegoldii |
| | FBI00191 | Clostridiaceae sp. FBI00191 |
| | FBI00194 | Ruminococcus faecis |
| | FBI00198 | Lachnoclostridium pacaense |
| | FBI00199 | Clostridium bolteae |
| | FBI00200 | Longicatena caecimuris |
| | FBI00201 | Eggerthella lenta |
| | FBI00205 | Blautia massiliensis |
| | FBI00206 | Bacteroides xylanisolvens |
| | FBI00211 | Bacteroides vulgatus |
| | FBI00220 | Megasphaera massiliensis |
| | FBI00221 | Butyricimonas faecihominis |
| | FBI00236 | Eisenbergiella tayi |
| | FBI00245 | Acidaminococcus intestini |
| | FBI00248 | Emergencia timonensis |
| | FBI00251 | Bifidobacterium pseudocatenulatum |
| | FBI00254 | Eubacterium hallii |
| | FBI00267 | Anaerofustis stercorihominis |
| | FBI00278 | Eubacterium ventriosum |
| | FBI00288 | Blautia hydrogenotrophica |
| | FBI00290 | Lachnospiraceae sp. FBI00290 |
| DS2 | FBI00004 | Acutalibacter timonensis |
| | FBI00012 | Alistipes onderdonkii |
| | FBI00015 | Bacteroides uniformis |
| | FBI00018 | Eubacterium rectale |
| | FBI00019 | Alistipes timonensis |
| | FBI00021 | Bacteroides kribbi |
| | FBI00038 | Coprococcus eutactus |
| | FBI00040 | Bilophila wadsworthia |
| | FBI00046 | Bacteroides caccae |
| | FBI00061 | Alistipes shahii |
| | FBI00066 | Parasutterella excrementihominis |
| | FBI00075 | Paraprevotella clara |
| | FBI00077 | Sutterella wadsworthensis |
| | FBI00080 | Sutterella massiliensis |
| | FBI00081 | Porphyromonas asaccharolytica |
| | FBI00085 | Ruminococcus bromii |
| | FBI00092 | Monoglobus pectinilyticus |
| | FBI00097 | Ruminococcaceae sp. FBI00097 |
| | FBI00099 | Gordonibacter pamelaeae |
| | FBI00112 | Bacteroides uniformis |
| | FBI00132 | Gordonibacter pamelaeae |
| | FBI00137 | Bacteroides fragilis |
| | FBI00140 | Phascolarctobacterium faecium |
| | FBI00149 | Monoglobus pectinilyticus |
| | FBI00151 | Clostridium aldenense |
| | FBI00176 | Ruthenibacterium lactatiformans |
| | FBI00189 | Bacteroides ovatus |
| | FBI00197 | Bifidobacterium bifidum |
| | FBI00208 | Anaerotruncus massiliensis |
| | FBI00212 | Clostridium aldenense |
| | FBI00224 | Sutterella wadsworthensis |
| | FBI00226 | Catabacter hongkongensis |
| | FBI00229 | Alistipes senegalensis |
| | FBI00233 | Ruminococcaceae sp. FBI00233 |
| | FBI00235 | Alistipes shahii |
| | FBI00237 | Dielma fastidiosa |
| | FBI00243 | Eubacterium siraeum |
| | FBI00244 | Faecalibacterium prausnitzii |
| | FBI00258 | Turicibacter sanguinis |
| | FBI00260 | Eubacterium rectale |
| | FBI00263 | Bacteroides caccae |
| | FBI00270 | Methanobrevibacter smithii |
| | FBI00273 | Barnesiella intestinihominis |
| | FBI00277 | Alistipes onderdonkii |
| | FBI00292 | Methanobrevibacter smithii |
| DS3 | FBI00009 | Bifidobacterium adolescentis |
| | FBI00011 | Bifidobacterium longum |
| | FBI00016 | Bifidobacterium pseudocatenulatum |
| | FBI00020 | Bacteroides thetaiotaomicron |
| | FBI00025 | Coprococcus comes |
| | FBI00027 | Fusicatenibacter saccharivorans |
| | FBI00030 | Eggerthella lenta |
| | FBI00047 | Eubacterium eligens |
| | FBI00052 | Bacteroides xylanisolvens |
| | FBI00053 | Lactobacillus rogosae |
| | FBI00056 | Clostridium citroniae |
| | FBI00062 | Collinsella aerofaciens |
| | FBI00078 | Blautia obeum |
| | FBI00096 | Eggerthella lenta |
| | FBI00104 | Blautia wexlerae |
| | FBI00110 | Lachnoclostridium pacaense |
| | FBI00111 | Bacteroides vulgatus |
| | FBI00113 | Parabacteroides merdae |
| | FBI00115 | Dorea formicigenerans |
| | FBI00116 | Ruminococcus faecis |
| | FBI00123 | Roseburia hominis |
| | FBI00124 | Anaero stipes hadrus |
| | FBI00126 | Bifidobacterium adolescentis |
| | FBI00135 | Bifidobacterium pseudocatenulatum |
| | FBI00147 | Clostridium bolteae |
| | FBI00159 | Eisenbergiella tayi |
| | FBI00167 | Dorea longicatena |
| | FBI00170 | Eggerthella lenta |
| | FBI00232 | Bacteroides stercoris |
| | FBI00255 | Hungatella hathewayi |
| | FBI00271 | Bacteroides xylanisolvens |
| DS4 | FBI00022 | Alistipes putredinis |
| | FBI00049 | Dialister succinatiphilus |
| | FBI00068 | Akkermansia muciniphila |
| | FBI00069 | Ruminococcus bromii |
| | FBI00152 | Dialister invisus |
| | FBI00165 | Bacteroides massiliensis |
| | FBI00171 | Bilophila wadsworthia |
| | FBI00175 | Holdemanella biformis |
| | FBI00177 | Parasutterella excrementihominis |
| | FBI00180 | Alistipes sp. FBI00180 |
| | FBI00182 | Bacteroides coprocola |
| | FBI00238 | Alistipes sp. FBI00238 |
| | FBI00269 | Alistipes putredinis |
| | FBI00274 | Eubacterium xylanophilum |
| | FBI00281 | Senegalimassilia anaerobia |
| DS5 (DS-OF1) | FBI00067 | Oxalobacter formigenes |
| DS6 (DS-OF2) | FBI00133 | Oxalobacter formigenes |
| DS7 (DS-OF3) | FBI00289 | Oxalobacter formigenes |

In certain embodiments, the Consortia comprises the microbiota that are at least 90% or at least 95% identical to those listed in any of Tables 1-19.

In certain embodiments, a Consortia comprises a microbial strain having a relative abundance of approximately 9000, 8000, 7000, 6000, 5000, 4000, 3000, 2000, 1000, 500, 10%, 0.100, 0.0100, 0.00100, 0.0001%, 0.00001%, or 0.000001% of the total microbial consortium. In certain embodiments, the relative abundance of a microbial strain is determined by metagenomic sequencing and calculated as the percentage of reads that are classified as an identified microbial strain, divided by the genome size. In certain embodiments, the relative abundance of a microbial strain of the present disclosure is determined by metagenomic shotgun sequencing.

### Active Microbes

The Consortia described herein comprise a plurality of active microbes capable of metabolizing a first metabolic substrate that causes or contributes to disease in an animal. In certain embodiments, the current disclosure provides a microbial consortium capable of metabolizing the first metabolic substrate at a pH within a range of 4 to 8. For example, in certain non-limiting embodiments, one or more than one of the plurality of active microbes is capable of metabolizing a first metabolic substrate at a pH within a range of about 4 to about 8, about 4.2 to about 8, about 4.4 to about 8, about 4.6 to about 8, about 4.8 to about 8, about 5 to about 8, about 5.2 to about 8, about 5.4 to about 8, about 5.6 to about 8, about 5.8 to about 8, about 6 to about 8, about 6.2 to about 8, about 6.4 to about 8, about 6.6 to about 8, about 6.8 to about 8, about 7 to about 8, about 7.2 to about 8, about 7.4 to about 8, about 7.6 to about 8, about 7.8 to about 8, about 4 to about 7, about 4.2 to about 7, about 4.4 to about 7, about 4.6 to about 7, about 4.8 to about 7, about 5 to about 7, about 5.2 to about 7, about 5.4 to about 7, about 5.6 to about 7, about 5.8 to about 7, about 6 to about 7, about 6.2 to about 7, about 6.4 to about 7, about 6.6 to about 7, about 6.8 to about 7, about 4 to about 6, about 4.2 to about 6, about 4.4 to about 6, about 4.6 to about 6, about 4.8 to about 6, about 5 to about 6, about 5.2 to about 6, about 5.4 to about 6, about 5.6 to about 6, about 5.8 to about 6, about 4 to about 6, about 4.2 to about 6, about 4.4 to about 6, about 4.6 to about 6, about 4.8 to about 6, about 5 to about 6, about 5.2 to about 6, about 5.4 to about 6, about 5.6 to about 6, or about 5.8 to about 6.

In certain embodiments, the plurality of active microbes comprises one microbial strain having a significantly different first metabolic substrate-metabolizing activity in a standard substrate-metabolizing assay conducted at two pH values differing by 1 pH unit and within a pH range of about 4 to about 8. In certain embodiments, the difference between the two pH values is about 1.0, about 1.1, about 1.2, about 1.3, about 1.4, about 1.5, about 1.6, about 1.7, about 1.8, about 1.9, about 2.0, about 2.1, about 2.2, about 2.3, about 2.4, about 2.5, about 2.6, about 2.7, about 2.8, about 2.9, about 3.0, about 3.2, about 3.3, about 3.4, about 3.5, about 3.6, about 3.7, about 3.8, about 3.9, or about 4.0 pH units. For example, in certain non-limiting embodiments, one microbial strain has significantly different first metabolic substrate-metabolizing activities in a standard substrate metabolizing assay at pH 4 and pH 8, pH 5 and pH 8, pH 6 and pH 8, pH 7 and pH 8, pH 4 and pH 7, pH 5 and pH 7, pH 6 and pH 7, pH 4 and pH 6, pH 5 and pH 6, or pH 4 and pH 5.

As used herein, “lower pH” or a “low pH” refers to a pH in a standardized substrate metabolization assay that is lower in value as compared to another pH value. For example, a standardized substrate metabolization assay performed at pH 4.5 has a lower pH as compared to a standardized substrate metabolization assay preformed at a pH of 7.5. “Higher pH,” as used herein, refers to a pH in a standardized substrate metabolization assay that is higher in value as compared to another pH value. For example a standardized substrate metabolization assay preformed at pH 7.5 has a higher pH as compared to a standardized substrate metabolization assay performed at a pH of 4.5.

As used herein, “higher first metabolic substrate-metabolizing activity” means either a first metabolic substrate-metabolizing activity of a microbial strain that is higher as compared to a first metabolic substrate-metabolizing activity of the same microbial strain under different conditions, and/or a first metabolic substrate-metabolizing activity of a microbial strain that is higher as compared to a first metabolic substrate-metabolizing activity of a different microbial strain under the same conditions.

In certain embodiments, the plurality of active microbes comprises two microbial strains having significantly different first metabolic substrate-metabolizing activities. For example, in certain non-limiting embodiments, one of the plurality of active microbes has a significantly higher first metabolic substrate-metabolizing activity at a lower pH as compared to the first metabolic substrate-metabolizing activity of another microbial strain in the plurality of active microbes at the same lower pH. In certain embodiments, one of the plurality of active microbes has a significantly higher first metabolic substrate-metabolizing activity at pH 4.0, 4.5, 5.0, 5.5, 6.0, or 6.5 as compared to the first metabolic substrate-metabolizing activity of another microbial strain in the plurality of active microbes at pH 4.0, 4.5, 5.0, 5.5, 6.0, or 6.5, respectively. In certain embodiments, one of the plurality of active microbes has a significantly higher first metabolic substrate-metabolizing activity at a higher pH as compared to the first metabolic substrate-metabolizing activity of another microbial strain in the plurality of active microbes at the same higher pH. In certain embodiments, one of the plurality of active microbes has a significantly higher first metabolic substrate-metabolizing activity at pH 7.5, 7.6. 7.7, 7.8, 7.9, or 8.0 as compared to the first metabolic substrate-metabolizing activity of another microbial strain in the plurality of active microbes at pH 7.5, 7.6, 7.7, 7.8, 7.9, or 8.0, respectively.

In certain embodiments, one of the plurality of active microbes has a significantly higher first metabolic substrate-metabolizing activity at a lower pH as compared to its first metabolic substrate-metabolizing activity at a higher pH. For example, in some embodiments one of the plurality of active microbes has a significantly higher first metabolic substrate-metabolizing activity at pH 4.0, 4.5, 5.0, 5.5, 6.0, or 6.5 than it does at pH 7.5, 7.6, 7.7, 7.8, 7.9, or 8.0. In certain embodiments, one of the plurality of active microbes has a significantly higher first metabolic substrate-metabolizing activity at a higher pH as compared to its first metabolic substrate-metabolizing activity at a lower pH. For example, in some embodiments one of the plurality of active microbes has a significantly higher first metabolic substrate-metabolizing activity at pH 7.5, 7.6. 7.7, 7.8, 7.9, or 8.0 than it does at pH 4.0, 4.5, 5.0, 5.5, 6.0, or 6.5.

In certain embodiments, the plurality of active microbes comprises an active microbe having a higher first metabolic substrate-metabolizing activity at a lower pH and another microbe having a higher first metabolic substrate-metabolizing activity at a higher pH. For example, in certain non-limiting embodiments, the plurality of active microbes comprises an active microbe having a higher first metabolic substrate-metabolizing activity at pH 4.0 and another microbe having a higher first metabolic substrate-metabolizing activity at pH 7.5. In certain embodiments, the plurality of active microbes comprises an active microbe having a higher first metabolic substrate-metabolizing activity at pH 4.0 and another microbe having a higher first metabolic substrate-metabolizing activity at pH 7.6. In certain embodiments, the plurality of active microbes comprises an active microbe having a higher first metabolic substrate-metabolizing activity at pH 4.0 and another microbe having a higher first metabolic substrate-metabolizing activity at pH 7.7. In certain embodiments, the plurality of active microbes comprises an active microbe having a higher first metabolic substrate-metabolizing activity at pH 4.0 and another microbe having a higher first metabolic substrate-metabolizing activity at pH 7.8. In certain embodiments, the plurality of active microbes comprises an active microbe having a higher first metabolic substrate-metabolizing activity at pH 4.0 and another microbe having a higher first metabolic substrate-metabolizing activity at pH 7.9. In certain embodiments, the plurality of active microbes comprises an active microbe having a higher first metabolic substrate-metabolizing activity at pH 4.0 and another microbe having a higher first metabolic substrate-metabolizing activity at pH 8.0. In certain embodiments, the plurality of active microbes comprises an active microbe having a higher first metabolic substrate-metabolizing activity at pH 4.5 and another microbe having a higher first metabolic substrate-metabolizing activity at pH 7.5. In certain embodiments, the plurality of active microbes comprises an active microbe having a higher first metabolic substrate-metabolizing activity at pH 4.5 and another microbe having a higher first metabolic substrate-metabolizing activity at pH 7.6. In certain embodiments, the plurality of active microbes comprises an active microbe having a higher first metabolic substrate-metabolizing activity at pH 4.5 and another microbe having a higher first metabolic substrate-metabolizing activity at pH 7.7. In certain embodiments, the plurality of active microbes comprises an active microbe having a higher first metabolic substrate-metabolizing activity at pH 4.5 and another microbe having a higher first metabolic substrate-metabolizing activity at pH 7.8. In certain embodiments, the plurality of active microbes comprises an active microbe having a higher first metabolic substrate-metabolizing activity at pH 4.5 and another microbe having a higher first metabolic substrate-metabolizing activity at pH 7.9. In certain embodiments, the plurality of active microbes comprises an active microbe having a higher first metabolic substrate-metabolizing activity at pH 4.5 and another microbe having a higher first metabolic substrate-metabolizing activity at pH 8.0. In certain embodiments, the plurality of active microbes comprises an active microbe having a higher first metabolic substrate-metabolizing activity at pH 5.0 and another microbe having a higher first metabolic substrate-metabolizing activity at pH 7.5. In certain embodiments, the plurality of active microbes comprises an active microbe having a higher first metabolic substrate-metabolizing activity at pH 5.0 and another microbe having a higher first metabolic substrate-metabolizing activity at pH 7.6. In certain embodiments, the plurality of active microbes comprises an active microbe having a higher first metabolic substrate-metabolizing activity at pH 5.0 and another microbe having a higher first metabolic substrate-metabolizing activity at pH 7.7. In certain embodiments, the plurality of active microbes comprises an active microbe having a higher first metabolic substrate-metabolizing activity at pH 5.0 and another microbe having a higher first metabolic substrate-metabolizing activity at pH 7.8. In certain embodiments, the plurality of active microbes comprises an active microbe having a higher first metabolic substrate-metabolizing activity at pH 5.0 and another microbe having a higher first metabolic substrate-metabolizing activity at pH 7.9. In certain embodiments, the plurality of active microbes comprises an active microbe having a higher first metabolic substrate-metabolizing activity at pH 5.0 and another microbe having a higher first metabolic substrate-metabolizing activity at pH 8.0. In certain embodiments, the plurality of active microbes comprises an active microbe having a higher first metabolic substrate-metabolizing activity at pH 5.5 and another microbe having a higher first metabolic substrate-metabolizing activity at pH 7.5. In certain embodiments, the plurality of active microbes comprises an active microbe having a higher first metabolic substrate-metabolizing activity at pH 5.5 and another microbe having a higher first metabolic substrate-metabolizing activity at pH 7.6. In certain embodiments, the plurality of active microbes comprises an active microbe having a higher first metabolic substrate-metabolizing activity at pH 5.5 and another microbe having a higher first metabolic substrate-metabolizing activity at pH 7.7. In certain embodiments, the plurality of active microbes comprises an active microbe having a higher first metabolic substrate-metabolizing activity at pH 5.5 and another microbe having a higher first metabolic substrate-metabolizing activity at pH 7.8. In certain embodiments, the plurality of active microbes comprises an active microbe having a higher first metabolic substrate-metabolizing activity at pH 5.5 and another microbe having a higher first metabolic substrate-metabolizing activity at pH 7.9. In certain embodiments, the plurality of active microbes comprises an active microbe having a higher first metabolic substrate-metabolizing activity at pH 5.5 and another microbe having a higher first metabolic substrate-metabolizing activity at pH 8.0. In certain embodiments, the plurality of active microbes comprises an active microbe having a higher first metabolic substrate-metabolizing activity at pH 6.0 and another microbe having a higher first metabolic substrate-metabolizing activity at pH 7.5. In certain embodiments, the plurality of active microbes comprises an active microbe having a higher first metabolic substrate-metabolizing activity at pH 6.0 and another microbe having a higher first metabolic substrate-metabolizing activity at pH 7.6. In certain embodiments, the plurality of active microbes comprises an active microbe having a higher first metabolic substrate-metabolizing activity at pH 6.0 and another microbe having a higher first metabolic substrate-metabolizing activity at pH 7.7. In certain embodiments, the plurality of active microbes comprises an active microbe having a higher first metabolic substrate-metabolizing activity at pH 6.0 and another microbe having a higher first metabolic substrate-metabolizing activity at pH 7.8. In certain embodiments, the plurality of active microbes comprises an active microbe having a higher first metabolic substrate-metabolizing activity at pH 6.0 and another microbe having a higher first metabolic substrate-metabolizing activity at pH 7.9. In certain embodiments, the plurality of active microbes comprises an active microbe having a higher first metabolic substrate-metabolizing activity at pH 6.0 and another microbe having a higher first metabolic substrate-metabolizing activity at pH 8.0. In certain embodiments, the plurality of active microbes comprises an active microbe having a higher first metabolic substrate-metabolizing activity at pH 6.5 and another microbe having a higher first metabolic substrate-metabolizing activity at pH 7.5. In certain embodiments, the plurality of active microbes comprises an active microbe having a higher first metabolic substrate-metabolizing activity at pH 6.5 and another microbe having a higher first metabolic substrate-metabolizing activity at pH 7.6. In certain embodiments, the plurality of active microbes comprises an active microbe having a higher first metabolic substrate-metabolizing activity at pH 6.5 and another microbe having a higher first metabolic substrate-metabolizing activity at pH 7.7. In certain embodiments, the plurality of active microbes comprises an active microbe having a higher first metabolic substrate-metabolizing activity at pH 6.5 and another microbe having a higher first metabolic substrate-metabolizing activity at pH 7.8. In certain embodiments, the plurality of active microbes comprises an active microbe having a higher first metabolic substrate-metabolizing activity at pH 6.5 and another microbe having a higher first metabolic substrate-metabolizing activity at pH 7.9. In certain embodiments, the plurality of active microbes comprises an active microbe having a higher first metabolic substrate-metabolizing activity at pH 6.5 and another microbe having a higher first metabolic substrate-metabolizing activity at pH 8.0.

In certain embodiments, the plurality of active microbes comprises one microbial strain having a significantly different first metabolic substrate-metabolizing activity in a standard substrate-metabolizing assay conducted at a first metabolic substrate concentration as compared to its first metabolic substrate-metabolizing activity in a standard substrate-metabolizing assay conducted at a different first metabolic substrate concentration, wherein the difference between the two first metabolic substrate concentrations is within a 100 fold range. In certain embodiments, the difference between the two first metabolic concentrations is about 1.2 fold. For example, in certain non-limiting embodiments, the difference between the two first metabolic substrate concentrations is at least about 1.2 fold, about 1.4 fold, about 1.6 fold, about 1.8 fold, about 2.0 fold, about 4 fold, about 6 fold, about 8 fold, about 10 fold, about 20 fold, about 30 fold, about 40 fold, about 50 fold, about 60 fold, about 70 fold, about 80 fold, about 90 fold, or about 100 fold or greater.

As used herein, “lower concentration of first metabolic substrate” refers to a substrate concentration in a standardized substrate metabolization assay that is lower in value as compared to another substrate concentration. “Higher concentration of first metabolic substrate,” as used herein, refers to a substrate concentration in a standardized substrate metabolization assay that is higher in value as compared to another substrate concentration.

In certain embodiments, the plurality of active microbes comprises two microbial strains having significantly different first metabolic substrate-metabolizing activities. For example, in certain non-limiting embodiments, one of the plurality of active microbes has a significantly higher first metabolic substrate-metabolizing activity at a lower concentration of first metabolic substrate as compared to the first metabolic substrate-metabolizing activity of another microbial strain in the plurality of active microbes at the same lower concentration of first metabolic substrate. In certain embodiments, one of the plurality of active microbes has a significantly higher first metabolic substrate-metabolizing activity at a higher concentration of first metabolic substrate as compared to the first metabolic substrate-metabolizing activity of another microbial strain in the plurality of active microbes at the same higher concentration of first metabolic substrate.

In certain embodiments, one of the plurality of active microbes has a significantly higher first metabolic substrate-metabolizing activity at a lower concentration of first metabolic substrate as compared to its first metabolic substrate-metabolizing activity at a higher concentration of first metabolic substrate. In certain embodiments, one of the plurality of active microbes has a significantly higher first metabolic substrate-metabolizing activity at a higher concentration of first metabolic substrate as compared to its first metabolic substrate-metabolizing activity at a lower concentration of first metabolic substrate.

In certain embodiments, the plurality of active microbes comprises an active microbe having a higher first metabolic substrate-metabolizing activity at a lower concentration of first metabolic substrate and another microbe having a higher first metabolic substrate-metabolizing activity at a higher concentration of first metabolic substrate. For example, in certain non-limiting embodiments, the difference between the lower concentration of first metabolic substrate and the higher concentration of first metabolic substrate is at least about 1.2 fold, about 1.4 fold, about 1.6 fold, about 1.8 fold, about 2.0 fold, about 4 fold, about 6 fold, about 8 fold, about 10 fold, about 20 fold, about 30 fold, about 40 fold, about 50 fold, about 60 fold, about 70 fold, about 80 fold, about 90 fold, or about 100 fold or greater.

In certain embodiments, the plurality of active microbes comprises two microbial strains having significantly different growth rates. For example, in certain non-limiting embodiments, one of the plurality of active microbes has a significantly higher growth rate at a lower pH as compared to the growth rate of another microbial strain in the plurality of active microbes at the same lower pH. In certain embodiments, one of the plurality of active microbes has a significantly higher growth rate at pH 4.0, 4.5, 5.0, 5.5, 6.0, or 6.5 as compared to the growth rate of another microbial strain in the plurality of active microbes at pH 4.0, 4.5, 5.0, 5.5, 6.0, or 6.5, respectively. In certain embodiments, one of the plurality of active microbes has a significantly higher growth rate at a higher pH as compared to the growth rate of another microbial strain in the plurality of active microbes at the same higher pH. In certain embodiments, one of the plurality of active microbes has a significantly higher growth rate at pH 7.5, 7.6. 7.7, 7.8, 7.9, or 8.0 as compared to the growth rate of another microbial strain in the plurality of active microbes at pH 7.5, 7.6, 7.7, 7.8, 7.9, or 8.0, respectively.

In certain embodiments, one of the plurality of active microbes has a significantly higher growth rate at a lower pH as compared to its growth rate at a higher pH. For example, in some embodiments one of the plurality of active microbes has a significantly higher growth rate at pH 4.0, 4.5, 5.0, 5.5, 6.0, or 6.5 than it does at pH 7.5, 7.6, 7.7, 7.8, 7.9, or 8.0. In certain embodiments, one of the plurality of active microbes has a significantly higher growth rate at a higher pH as compared to its growth rate at a lower pH. For example, in some embodiments one of the plurality of active microbes has a significantly higher growth rate at pH 7.5, 7.6. 7.7, 7.8, 7.9, or 8.0 than it does at pH 4.0, 4.5, 5.0, 5.5, 6.0, or 6.5.

In certain embodiments, the plurality of active microbes comprises one microbial strain having a significantly higher growth rate when cultured in media containing a certain concentration of first metabolic substrate concentration as compared to the growth rate of another microbial strain in the plurality of active microbes cultured in the same media containing the same concentration of the first metabolic substrate. In certain embodiments, the difference between the two growth rates is at least about 0.2 fold, at least about 0.4 fold, at least about 0.6 fold, at least about 0.8 fold, at least about 1.0 fold, at least about 1.2 fold, at least about 1.4 fold, at least about 1.6 fold, at least about 1.8 fold, or at least about 2.0 fold.

In certain embodiments, the first metabolic substrate may be selected from, but not limited to, oxalate and a bile acid (e.g., lithocholic acid (LCA), deoxycholic acid (DCA)).

In certain embodiments, the current disclosure provides a microbial consortium comprising a plurality of active microbes capable of metabolizing a first metabolic substrate to one or more than one metabolite. For example, in certain non-limiting embodiments, the one or more than one metabolite may be selected from, but not limited to, formate, CO₂, and a secondary bile acid (e.g., 3-oxo-deoxycholic acid (3 oxoDCA), 3-oxo-lithocholic acid (3oxoLCA), iso-lithocholic acid (iso-LCA), or iso-deoxycholic acid (iso-DCA)). In certain embodiments, the plurality of active microbes can comprise 2 to 200 microbial strains. For example, in certain non-limiting embodiments, a microbial consortium comprises 2 to 10, 2 to 15, 2 to 20, 2 to 25, 2 to 30, 2 to 35, 2 to 40, 2 to 45, 2 to 50, 2 to 75, 2 to 100, 2 to 125, 2 to 150, 2 to 175, or 2 to 200 active microbial strains. In certain embodiments, the plurality of active microbes can comprise 2 to 20 microbial strains.

### Oxalate-Metabolizing Active Microbes

The Consortia described herein comprise a plurality of active microbes that metabolize oxalate. In certain embodiments, each of the plurality of active microbes that metabolize oxalate express sufficient amounts of one or more than one enzyme involved in oxalate metabolism. For example, in certain non-limiting embodiments, one or more than one active microbe expresses formyl-CoA transferase (Frc), an oxalate-formate antiporter (e.g., OxIT), and oxalyl-CoA decarboxylase (e.g., OxC), and/or oxalate decarboxylase (e.g., OxD).

In certain embodiments, the plurality of active microbes that metabolize oxalate comprise 2 to 20 oxalate-metabolizing microbial strains. In certain embodiments, the plurality of active microbes that metabolize oxalate comprise 2 to 5 oxalate-metabolizing microbial strains. In certain embodiments, the plurality of active microbes that metabolize oxalate comprise 2 to 7 oxalate-metabolizing microbial strains. In certain embodiments, the plurality of active microbes that metabolize oxalate comprise 2 to 7 oxalate-metabolizing microbial strains. In certain embodiments, the plurality of active microbes that metabolize oxalate comprise more than 20 oxalate-metabolizing microbial strains. In certain embodiments, the plurality of active microbes comprises 3 strains of oxalate-metabolizing microbes. In certain embodiments, 2 or more of the active microbes are different strains of the same species.

In certain embodiments, the plurality of active microbes that metabolize oxalate may comprise one or more microbial species selected from, but not limited to Oxalobacter formigenes, Bifidobacterium sp., Bifidobacterium dentium, Dialister invisus, Lactobacillus acidophilus, Lactobacillus gasseri, Lactobacillus helveticus, Lactobacillus reuteri, Eggerthella lenta, Lactobacillus rhamnosus, Enterococcus faecalis, Enterococcus gallinarum, Enterococcus faecium, Providencia rettgeri, Streptococcus thermophilus, Lactobacillus plantarum, Lactobacillus casei, Lactobacillus salivarius, Lactobacillus johnsii, Bifidobacterium infantis, Bifidobacterium animalis, Clostridium sporogenes, Leuconostoc lactis, or Leuconostoc mesenteroides.

In certain embodiments, the Consortia described herein comprise 3 strains of Oxalobacter formigenes. In certain embodiments, the Consortia described herein comprise 3 strains of Oxalobacter formigenes, each with different phenotypic properties. In certain embodiments, the Consortia described herein comprise 3 strains of Oxalobacter formigenes wherein 1 strain is low pH tolerant, 1 strain is high oxalate tolerant, and 1 strain has a high growth rate. In certain embodiments, the low pH tolerance is approximately pH 5. In certain embodiments, the high oxalate tolerance is approximately 150 mM. In certain embodiments, the high oxalate tolerance is approximately 15 mM.

In certain embodiments, the plurality of active microbes comprises an Oxalobacter formigenes strain having a 16S sequence at least about 80% identical to the nucleotide sequence set forth in SEQ ID NO: 42, SEQ ID NO: 79, or SEQ ID NO: 146. In certain embodiments, the plurality of active microbes comprises an Oxalobacter formigenes strain having a 16S sequence at least about 90%, at least about 91%, at least about 92%, at least about 93%, at least about 94%, at least about 95%, at least about 96%, at least about 97%, at least about 98%, or at least about 99% identical to the nucleotide sequence set forth in SEQ ID NO: 42, SEQ ID NO: 79, or SEQ ID NO: 146. In certain embodiments, the plurality of active microbes comprises three Oxalobacter formigenes strains, wherein the first, second, and third have a respective 16S sequence that is identical to the nucleotide sequence set forth in SEQ ID NO: 42, SEQ ID NO: 79, or SEQ ID NO: 146. In certain embodiments, the plurality of active microbes comprises three Oxalobacter formigenes strains, wherein the first, second, and third have a respective 16S sequence that is at least about 90%, at least about 91%, at least about 92%, at least about 93%, at least about 94%, at least about 95%, at least about 96%, at least about 97%, at least about 98%, or at least about 99% identical to the nucleotide sequence set forth in SEQ ID NO: 42, SEQ ID NO: 79, or SEQ ID NO: 146.

In certain embodiments, the plurality of active microbes comprises three Oxalobacter formigenes strains, wherein the first, second, and third have a respective 16S sequence that is at least about 97% identical to the nucleotide sequence set forth in SEQ ID NO: 42, SEQ ID NO: 79, or SEQ ID NO: 146.

In some embodiments the plurality of active microbes comprises an Oxalobacter formigenes strain having a 16S sequence at least about 80% identical to the nucleotide sequence set forth in SEQ ID NO: 42 and an Oxalobacter formigenes strain having a 16S sequence at least about 80% identical to the nucleotide sequence set forth in SEQ ID NO: 79. In certain embodiments, the plurality of active microbes comprises an Oxalobacter formigenes strain having a 16S sequence at least about 90%, at least about 91%, at least about 92%, at least about 93%, at least about 94%, at least about 95%, at least about 96%, at least about 97%, at least about 98%, or at least about 99%, identical to the nucleotide sequence set forth in SEQ ID NO: 42 and an Oxalobacter formigenes strain having a 16S sequence at least about 90%, at least about 91%, at least about 92%, at least about 93%, at least about 94%, at least about 95%, at least about 96%, at least about 97%, at least about 98%, or at least about 99% identical to the nucleotide sequence set forth in SEQ ID NO: 79.

In some embodiments the plurality of active microbes comprises an Oxalobacter formigenes strain having a 16S sequence at least about 80% identical to the nucleotide sequence set forth in SEQ ID NO: 42 and an Oxalobacter formigenes strain having a 16S sequence at least about 80% identical to the nucleotide sequence set forth in SEQ ID NO: 146. In certain embodiments, the plurality of active microbes comprises an Oxalobacter formigenes strain having a 16S sequence at least about 90%, at least about 91%, at least about 92%, at least about 93%, at least about 94%, at least about 95%, at least about 96%, at least about 97%, at least about 98%, or at least about 99% identical to the nucleotide sequence set forth in SEQ ID NO: 42 and an Oxalobacter formigenes strain having a 16S sequence at least about 90%, at least about 91%, at least about 92%, at least about 93%, at least about 94%, at least about 95%, at least about 96%, at least about 97%, at least about 98%, or at least about 99% identical to the nucleotide sequence set forth in SEQ ID NO: 146.

In some embodiments the plurality of active microbes comprises an Oxalobacter formigenes strain having a 16S sequence at least 80% identical to the nucleotide sequence set forth in SEQ ID NO: 79 and an Oxalobacter formigenes strain having a 16S sequence at least about 80% identical to the nucleotide sequence set forth in SEQ ID NO: 146. In certain embodiments, the plurality of active microbes comprises an Oxalobacter formigenes strain having a 16S sequence at least about 90%, at least about 91%, at least about 92%, at least about 93%, at least about 94%, at least about 95%, at least about 96%, at least about 97%, at least about 98%, or at least about 99% identical to the nucleotide sequence set forth in SEQ ID NO: 79 and an Oxalobacter formigenes strain having a 16S sequence at least about 90%, at least about 91%, at least about 92%, at least about 93%, at least about 94%, at least about 95%, at least about 96%, at least about 97%, at least about 98%, or at least about 99% identical to the nucleotide sequence set forth in SEQ ID NO: 146.

As used herein, “substantially metabolizing oxalate,” “substantial metabolization of oxalate,” and variants thereof, refer to a statistically significant reduction in the amount of oxalate in an in vitro assay. In certain embodiments, one or more than one of the plurality of active microbes is capable of substantially metabolizing oxalate at a pH within a range of 4 to 8. In certain embodiments, one or more than one of the plurality of active microbes is capable of metabolizing oxalate at a pH within a range of about 4 to about 8, about 4.2 to about 8, about 4.4 to about 8, about 4.6 to about 8, about 4.8 to about 8, about 5 to about 8, about 5.2 to about 8, about 5.4 to about 8, about 5.6 to about 8, about 5.8 to about 8, about 6 to about 8, about 6.2 to about 8, about 6.4 to about 8, about 6.6 to about 8, about 6.8 to about 8, about 7 to about 8, about 7.2 to about 8, about 7.4 to about 8, about 7.6 to about 8, about 7.8 to about 8, about 4 to about 7, about 4.2 to about 7, about 4.4 to about 7, about 4.6 to about 7, about 4.8 to about 7, about 5 to about 7, about 5.2 to about 7, about 5.4 to about 7, about 5.6 to about 7, about 5.8 to about 7, about 6 to about 7, about 6.2 to about 7, about 6.4 to about 7, about 6.6 to about 7, about 6.8 to about 7, about 4 to about 6, about 4.2 to about 6, about 4.4 to about 6, about 4.6 to about 6, about 4.8 to about 6, about 5 to about 6, about 5.2 to about 6, about 5.4 to about 6, about 5.6 to about 6, about 5.8 to about 6, about 4 to about 6, about 4.2 to about 6, about 4.4 to about 6, about 4.6 to about 6, about 4.8 to about 6, about 5 to about 6, about 5.2 to about 6, about 5.4 to about 6, about 5.6 to about 6, or about 5.8 to about 6.

In certain embodiments, the plurality of active microbes comprises one microbial strain having a significantly different oxalate-metabolizing activity in a standard oxalate metabolizing assay conducted at two pH values differing by at least 1 pH unit and within a pH range of 4 to 8. In certain embodiments, one microbial strain has significantly different oxalate-metabolizing activities in a standard oxalate metabolizing assay at pH 4 and pH 8, pH 5 and pH 8, pH 6 and pH 8, pH 7 and pH 8, pH 4 and pH 7, pH 5 and pH 7, pH 6 and pH 7, pH 4 and pH 6, pH 5 and pH 6, or pH 4 and pH 5.

In certain embodiments, oxalate-metabolizing activity is detected using a standard oxalate metabolization assay. In certain embodiments, oxalate-metabolizing activity is detected using a colorimetric enzyme assay that measures the activity of oxalate oxidase. In certain embodiments, relative changes in oxalate abundance in culture media inoculated with microbial strains are measured using a commercial oxalate assay kit (e.g., Sigma-Aldrich, Catalog #MAK315). In certain embodiments, oxalate-metabolizing activity is detected using liquid chromatography-mass spectrometry (LC-MS/MS). In certain embodiments, relative changes in oxalate abundance is compared between the abundance of oxalate at the beginning of incubation (i.e. t=0), and after about 2 hours, about 4 hours, about 6 hours, about 8, hours, about 10 hours, about 12 hours, about 14 hours, about 16 hours, about 18 hours, about 24 hours, about 30 hours, about 36 hours, about 48 hours, about 60 hours, about 72 hours, about 84 hours, about 96 hours, about 120 hours, or about 144 hours incubation.

As used herein, “higher oxalate metabolizing activity” means either an oxalate metabolizing activity of a microbial strain that is higher as compared to an oxalate metabolizing activity of the same microbial strain under different conditions, and/or an oxalate metabolizing activity of a microbial strain that is higher as compared to an oxalate metabolizing activity of a different microbial strain under the same conditions.

In certain embodiments, the plurality of active microbes comprises two microbial strains having significantly different oxalate metabolizing activities. In certain embodiments, one of the plurality of active microbes has a significantly higher oxalate metabolizing activity at a lower pH as compared to the oxalate metabolizing activity of another microbial strain in the plurality of active microbes at the same lower pH. In certain embodiments, one of the plurality of active microbes has a significantly higher oxalate metabolizing activity at pH 4.0, 4.5, 5.0, 5.5, 6.0, or 6.5 as compared to the oxalate metabolizing activity of another microbial strain in the plurality of active microbes at pH 4.0, 4.5, 5.0, 5.5, 6.0, or 6.5, respectively. In certain embodiments, one of the plurality of active microbes has a significantly higher oxalate metabolizing activity at a higher pH as compared to the oxalate metabolizing activity of another microbial strain in the plurality of active microbes at the same higher pH. In certain embodiments, one of the plurality of active microbes has a significantly higher oxalate metabolizing activity at pH 7.5, 7.6. 7.7, 7.8, 7.9, or 8.0 as compared to the oxalate metabolizing activity of another microbial strain in the plurality of active microbes at pH 7.5, 7.6. 7.7, 7.8, 7.9, or 8.0, respectively.

In certain embodiments, one of the plurality of active microbes has a significantly higher oxalate metabolizing activity at a lower pH as compared to its oxalate metabolizing activity at a higher pH. In certain embodiments one of the plurality of active microbes has a significantly higher oxalate metabolizing activity at pH 4.0, 4.5, 5.0, 5.5, 6.0, or 6.5 than it does at pH 7.5, 7.6. 7.7, 7.8, 7.9, or 8.0. In certain embodiments, one of the plurality of active microbes has a significantly higher oxalate metabolizing activity at a higher pH as compared to its oxalate metabolizing activity at a lower pH. In certain embodiments one of the plurality of active microbes has a significantly higher oxalate metabolizing activity at pH 7.5, 7.6. 7.7, 7.8, 7.9, or 8.0 than it does at pH 4.0, 4.5, 5.0, 5.5, 6.0, or 6.5.

In certain embodiments, the plurality of active microbes comprises an active microbe having a higher oxalate metabolizing activity at a lower pH and another microbe having a higher oxalate metabolizing activity at a higher pH. In certain embodiments, the plurality of active microbes comprises an active microbe having a higher oxalate metabolizing activity at pH 4.0 and another microbe having a higher oxalate metabolizing activity at pH 7.5. In certain embodiments, the plurality of active microbes comprises an active microbe having a higher oxalate metabolizing activity at pH 4.0 and another microbe having a higher oxalate metabolizing activity at pH 7.6. In certain embodiments, the plurality of active microbes comprises an active microbe having a higher oxalate metabolizing activity at pH 4.0 and another microbe having a higher oxalate metabolizing activity at pH 7.7. In certain embodiments, the plurality of active microbes comprises an active microbe having a higher oxalate metabolizing activity at pH 4.0 and another microbe having a higher oxalate metabolizing activity at pH 7.8. In certain embodiments, the plurality of active microbes comprises an active microbe having a higher oxalate metabolizing activity at pH 4.0 and another microbe having a higher oxalate metabolizing activity at pH 7.9. In certain embodiments, the plurality of active microbes comprises an active microbe having a higher oxalate metabolizing activity at pH 4.0 and another microbe having a higher oxalate metabolizing activity at pH 8.0. In certain embodiments, the plurality of active microbes comprises an active microbe having a higher oxalate metabolizing activity at pH 4.5 and another microbe having a higher oxalate metabolizing activity at pH 7.5. In certain embodiments, the plurality of active microbes comprises an active microbe having a higher oxalate metabolizing activity at pH 4.5 and another microbe having a higher oxalate metabolizing activity at pH 7.6. In certain embodiments, the plurality of active microbes comprises an active microbe having a higher oxalate metabolizing activity at pH 4.5 and another microbe having a higher oxalate metabolizing activity at pH 7.7. In certain embodiments, the plurality of active microbes comprises an active microbe having a higher oxalate metabolizing activity at pH 4.5 and another microbe having a higher oxalate metabolizing activity at pH 7.8. In certain embodiments, the plurality of active microbes comprises an active microbe having a higher oxalate metabolizing activity at pH 4.5 and another microbe having a higher oxalate metabolizing activity at pH 7.9. In certain embodiments, the plurality of active microbes comprises an active microbe having a higher oxalate metabolizing activity at pH 4.5 and another microbe having a higher oxalate metabolizing activity at pH 8.0. In certain embodiments, the plurality of active microbes comprises an active microbe having a higher oxalate metabolizing activity at pH 5.0 and another microbe having a higher oxalate metabolizing activity at pH 7.5. In certain embodiments, the plurality of active microbes comprises an active microbe having a higher oxalate metabolizing activity at pH 5.0 and another microbe having a higher oxalate metabolizing activity at pH 7.6. In certain embodiments, the plurality of active microbes comprises an active microbe having a higher oxalate metabolizing activity at pH 5.0 and another microbe having a higher oxalate metabolizing activity at pH 7.7. In certain embodiments, the plurality of active microbes comprises an active microbe having a higher oxalate metabolizing activity at pH 5.0 and another microbe having a higher oxalate metabolizing activity at pH 7.8. In certain embodiments, the plurality of active microbes comprises an active microbe having a higher oxalate metabolizing activity at pH 5.0 and another microbe having a higher oxalate metabolizing activity at pH 7.9. In certain embodiments, the plurality of active microbes comprises an active microbe having a higher oxalate metabolizing activity at pH 5.0 and another microbe having a higher oxalate metabolizing activity at pH 8.0. In certain embodiments, the plurality of active microbes comprises an active microbe having a higher oxalate metabolizing activity at pH 5.5 and another microbe having a higher oxalate metabolizing activity at pH 7.5. In certain embodiments, the plurality of active microbes comprises an active microbe having a higher oxalate metabolizing activity at pH 5.5 and another microbe having a higher oxalate metabolizing activity at pH 7.6. In certain embodiments, the plurality of active microbes comprises an active microbe having a higher oxalate metabolizing activity at pH 5.5 and another microbe having a higher oxalate metabolizing activity at pH 7.7. In certain embodiments, the plurality of active microbes comprises an active microbe having a higher oxalate metabolizing activity at pH 5.5 and another microbe having a higher oxalate metabolizing activity at pH 7.8. In certain embodiments, the plurality of active microbes comprises an active microbe having a higher oxalate metabolizing activity at pH 5.5 and another microbe having a higher oxalate metabolizing activity at pH 7.9. In certain embodiments, the plurality of active microbes comprises an active microbe having a higher oxalate metabolizing activity at pH 5.5 and another microbe having a higher oxalate metabolizing activity at pH 8.0. In certain embodiments, the plurality of active microbes comprises an active microbe having a higher oxalate metabolizing activity at pH 6.0 and another microbe having a higher oxalate metabolizing activity at pH 7.5. In certain embodiments, the plurality of active microbes comprises an active microbe having a higher oxalate metabolizing activity at pH 6.0 and another microbe having a higher oxalate metabolizing activity at pH 7.6. In certain embodiments, the plurality of active microbes comprises an active microbe having a higher oxalate metabolizing activity at pH 6.0 and another microbe having a higher oxalate metabolizing activity at pH 7.7. In certain embodiments, the plurality of active microbes comprises an active microbe having a higher oxalate metabolizing activity at pH 6.0 and another microbe having a higher oxalate metabolizing activity at pH 7.8. In certain embodiments, the plurality of active microbes comprises an active microbe having a higher oxalate metabolizing activity at pH 6.0 and another microbe having a higher oxalate metabolizing activity at pH 7.9. In certain embodiments, the plurality of active microbes comprises an active microbe having a higher oxalate metabolizing activity at pH 6.0 and another microbe having a higher oxalate metabolizing activity at pH 8.0. In certain embodiments, the plurality of active microbes comprises an active microbe having a higher oxalate metabolizing activity at pH 6.5 and another microbe having a higher oxalate metabolizing activity at pH 7.5. In certain embodiments, the plurality of active microbes comprises an active microbe having a higher oxalate metabolizing activity at pH 6.5 and another microbe having a higher oxalate metabolizing activity at pH 7.6. In certain embodiments, the plurality of active microbes comprises an active microbe having a higher oxalate metabolizing activity at pH 6.5 and another microbe having a higher oxalate metabolizing activity at pH 7.7. In certain embodiments, the plurality of active microbes comprises an active microbe having a higher oxalate metabolizing activity at pH 6.5 and another microbe having a higher oxalate metabolizing activity at pH 7.8. In certain embodiments, the plurality of active microbes comprises an active microbe having a higher oxalate metabolizing activity at pH 6.5 and another microbe having a higher oxalate metabolizing activity at pH 7.9. In certain embodiments, the plurality of active microbes comprises an active microbe having a higher oxalate metabolizing activity at pH 6.5 and another microbe having a higher oxalate metabolizing activity at pH 8.0.

In certain embodiments, one or more than one of the plurality of active microbes is capable of substantially metabolizing oxalate at an oxalate concentration of about 0.75 mM to about 40 mM of oxalate. In certain embodiments, one or more than one of the plurality of active microbes is capable of substantially metabolizing oxalate at an oxalate concentration within a range of about 0.75 mM to about 40 mM, of about 1 mM to about 40 mM, of about 2.5 mM to about 40 mM, of about 5 mM to about 40 mM, of about 7.5 mM to about 40 mM, of about 10 mM to about 40 mM, of about 15 mM to about 40 mM, of about 20 mM to about 40 mM, of about 25 mM to about 40 mM, of about 30 mM to about 40 mM, of about 0.75 mM to about 30 mM, of about 1 mM to about 30 mM, of about 2.5 mM to about 30 mM, of about 5 mM to about 30 mM, of about 7.5 mM to about 30 mM, of about 10 mM to about 30 mM, of about 15 mM to about 30 mM, of about 20 mM to about 30 mM, of about 25 mM to about 30 mM, of about 0.75 mM to about 25 mM, of about 1 mM to about 25 mM, of about 2.5 mM to about 25 mM, of about 5 mM to about 25 mM, of about 7.5 mM to about 25 mM, of about 10 mM to about 25 mM, of about 15 mM to about 25 mM, of about 20 mM to about 25 mM, of about 0.75 mM to about 20 mM, of about 1 mM to about 20 mM, of about 2.5 mM to about 20 mM, of about 5 mM to about 20 mM, of about 7.5 mM to about 20 mM, of about 10 mM to about 20 mM, of about 15 mM to about 20 mM, of about 0.75 mM to about 15 mM, of about 1 mM to about 15 mM, of about 2.5 mM to about 15 mM, of about 5 mM to about 15 mM, of about 7.5 mM to about 15 mM, of about 10 mM to about 15 mM, of about 0.75 mM to about 10 mM, of about 1 mM to about 10 mM, of about 2.5 mM to about 10 mM, of about 5 mM to about 10 mM, of about 7.5 mM to about 10 mM, of about 0.75 mM to about 5 mM, of about 1 mM to about 5 mM, of about 2.5 mM to about 5 mM, or of about 0.75 mM to about 1 mM.

In certain embodiments, the plurality of active microbes comprises one microbial strain having a significantly different oxalate-metabolizing activity in a standard in vitro oxalate metabolizing assay at an oxalate concentration as compared to its oxalate-metabolizing activity in a standard in vitro oxalate metabolizing assay conducted at a different oxalate concentration, wherein the difference between the two oxalate concentrations is within 100 fold. In certain embodiments, one microbial strain has significantly different oxalate-metabolizing activities in a standard oxalate metabolizing assay conducted at about 0.75 mM oxalate and about 40 mM oxalate, about 1 mM and about 40 mM, about 2.5 mM and about 40 mM, about 5 mM and about 40 mM, about 7.5 mM and about 40 mM, about 10 mM and about 40 mM, about 15 mM and about 40 mM, about 20 mM and about 40 mM, about 25 mM and about 40 mM, about 30 mM and about 40 mM, about 0.75 mM and about 30 mM, about 1 mM and about 30 mM, about 2.5 mM and about 30 mM, about 5 mM and about 30 mM, about 7.5 mM and about 30 mM, about 10 mM and about 30 mM, about 15 mM and about 30 mM, about 20 mM and about 30 mM, about 25 mM and about 30 mM, about 0.75 mM and about 25 mM, about 1 mM and about 25 mM, about 2.5 mM and about 25 mM, about 5 mM and about 25 mM, about 7.5 mM and about 25 mM, about 10 mM and about 25 mM, about 15 mM and about 25 mM, about 20 mM and about 25 mM, about 0.75 mM and about 20 mM, about 1 mM and about 20 mM, about 2.5 mM and about 20 mM, about 5 mM and about 20 mM, about 7.5 mM and about 20 mM, about 10 mM and about 20 mM, about 15 mM and about 20 mM, about 0.75 mM and about 15 mM, about 1 mM and about 15 mM, about 2.5 mM and about 15 mM, about 5 mM and about 15 mM, about 7.5 mM and about 15 mM, about 10 mM and about 15 mM, about 0.75 mM and about 10 mM, about 1 mM and about 10 mM, about 2.5 mM and about 10 mM, about 5 mM and about 10 mM, about 7.5 mM and about 10 mM, about 0.75 mM and about 5 mM, about 1 mM and about 5 mM, about 2.5 mM and about 5 mM, or about 0.75 mM and about 1 mM.

In certain embodiments, the plurality of active microbes comprises two microbial strains having significantly different oxalate metabolizing activities. In certain embodiments, one of the plurality of active microbes has a significantly higher oxalate metabolizing activity at a lower concentration of oxalate as compared to the oxalate metabolizing activity of another microbial strain in the plurality of active microbes at the same lower concentration of oxalate. In certain embodiments, one of the plurality of active microbes has a significantly higher oxalate metabolizing activity at an oxalate concentration of about 0.75 mM, about 1 mM, about 2.5 mM, about 5 mM, or about 7.5 mM, as compared to the oxalate metabolizing activity of another microbial strain in the plurality of active microbes at an oxalate concentration of about 0.75 mM, about 1 mM, about 2.5 mM, about 5 mM, or about 7.5 mM, respectively. In certain embodiments, one of the plurality of active microbes has a significantly higher oxalate metabolizing activity at a higher concentration of oxalate as compared to the oxalate metabolizing activity of another microbial strain in the plurality of active microbes at the same higher concentration of oxalate. In certain embodiments, one of the plurality of active microbes has a significantly higher oxalate metabolizing activity at an oxalate concentration of about 15 mM, about 20 mM, about 25 mM, about 30 mM, or about 40 mM as compared to the oxalate metabolizing activity of another microbial strain in the plurality of active microbes at an oxalate concentration of about 15 mM, about 20 mM, about 25 mM, about 30 mM, or about 40 mM, respectively.

In certain embodiments, one of the plurality of active microbes has a significantly higher oxalate metabolizing activity at a lower oxalate concentration as compared to its oxalate metabolizing activity at a higher oxalate concentration. In certain embodiments one of the plurality of active microbes has a significantly higher oxalate metabolizing activity at about 0.75 mM, about 1 mM, about 2.5 mM, about 5 mM, or about 7.5 mM of oxalate than it does at about 15 mM, about 20 mM, about 25 mM, about 30 mM, or about 40 mM of oxalate. In certain embodiments, one of the plurality of active microbes has a significantly higher oxalate metabolizing activity at a higher oxalate concentration as compared to its oxalate metabolizing activity at a lower oxalate concentration. In certain embodiments one of the plurality of active microbes has a significantly higher oxalate metabolizing activity at about 15 mM, about 20 mM, about 25 mM, about 30 mM, or about 40 mM of oxalate than it does at about 0.75 mM, about 1 mM, about 2.5 mM, about 5 mM, or about 7.5 mM of oxalate.

In certain embodiments, the plurality of active microbes comprises an active microbe having a higher oxalate metabolizing activity at a lower concentration of oxalate and another microbe having a higher oxalate metabolizing activity at a higher concentration of oxalate. In certain embodiments, the plurality of active microbes comprises an active microbe having a higher oxalate metabolizing activity at about 0.75 mM oxalate and another active microbe having a higher oxalate metabolizing activity at about 40 mM oxalate. In certain embodiments, the plurality of active microbes comprises an active microbe having a higher oxalate metabolizing activity at about 1 mM oxalate and another active microbe having a higher oxalate metabolizing activity at about 40 mM oxalate. In certain embodiments, the plurality of active microbes comprises an active microbe having a higher oxalate metabolizing activity at about 2.5 mM oxalate and another active microbe having a higher oxalate metabolizing activity at about 40 mM oxalate. In certain embodiments, the plurality of active microbes comprises an active microbe having a higher oxalate metabolizing activity at about 5 mM oxalate and another active microbe having a higher oxalate metabolizing activity at about 40 mM oxalate. In certain embodiments, the plurality of active microbes comprises an active microbe having a higher oxalate metabolizing activity at about 7.5 mM oxalate and another active microbe having a higher oxalate metabolizing activity at about 40 mM oxalate. In certain embodiments, the plurality of active microbes comprises an active microbe having a higher oxalate metabolizing activity at about 0.75 mM oxalate and another active microbe having a higher oxalate metabolizing activity at about 30 mM oxalate. In certain embodiments, the plurality of active microbes comprises an active microbe having a higher oxalate metabolizing activity at about 1 mM oxalate and another active microbe having a higher oxalate metabolizing activity at about 30 mM oxalate. In certain embodiments, the plurality of active microbes comprises an active microbe having a higher oxalate metabolizing activity at about 2.5 mM oxalate and another active microbe having a higher oxalate metabolizing activity at about 30 mM oxalate. In certain embodiments, the plurality of active microbes comprises an active microbe having a higher oxalate metabolizing activity at about 5 mM oxalate and another active microbe having a higher oxalate metabolizing activity at about 30 mM oxalate. In certain embodiments, the plurality of active microbes comprises an active microbe having a higher oxalate metabolizing activity at about 7.5 mM oxalate and another active microbe having a higher oxalate metabolizing activity at about 30 mM oxalate. In certain embodiments, the plurality of active microbes comprises an active microbe having a higher oxalate metabolizing activity at about 0.75 mM oxalate and another active microbe having a higher oxalate metabolizing activity at about 25 mM oxalate. In certain embodiments, the plurality of active microbes comprises an active microbe having a higher oxalate metabolizing activity at about 1 mM oxalate and another active microbe having a higher oxalate metabolizing activity at about 25 mM oxalate. In certain embodiments, the plurality of active microbes comprises an active microbe having a higher oxalate metabolizing activity at about 2.5 mM oxalate and another active microbe having a higher oxalate metabolizing activity at about 25 mM oxalate. In certain embodiments, the plurality of active microbes comprises an active microbe having a higher oxalate metabolizing activity at about 5 mM oxalate and another active microbe having a higher oxalate metabolizing activity at about 25 mM oxalate. In certain embodiments, the plurality of active microbes comprises an active microbe having a higher oxalate metabolizing activity at about 7.5 mM oxalate and another active microbe having a higher oxalate metabolizing activity at about 25 mM oxalate. In certain embodiments, the plurality of active microbes comprises an active microbe having a higher oxalate metabolizing activity at about 0.75 mM oxalate and another active microbe having a higher oxalate metabolizing activity at about 20 mM oxalate. In certain embodiments, the plurality of active microbes comprises an active microbe having a higher oxalate metabolizing activity at about 1 mM oxalate and another active microbe having a higher oxalate metabolizing activity at about 20 mM oxalate. In certain embodiments, the plurality of active microbes comprises an active microbe having a higher oxalate metabolizing activity at about 2.5 mM oxalate and another active microbe having a higher oxalate metabolizing activity at about 20 mM oxalate. In certain embodiments, the plurality of active microbes comprises an active microbe having a higher oxalate metabolizing activity at about 5 mM oxalate and another active microbe having a higher oxalate metabolizing activity at about 20 mM oxalate. In certain embodiments, the plurality of active microbes comprises an active microbe having a higher oxalate metabolizing activity at about 7.5 mM oxalate and another active microbe having a higher oxalate metabolizing activity at about 20 mM oxalate. In certain embodiments, the plurality of active microbes comprises an active microbe having a higher oxalate metabolizing activity at about 0.75 mM oxalate and another active microbe having a higher oxalate metabolizing activity at about 15 mM oxalate. In certain embodiments, the plurality of active microbes comprises an active microbe having a higher oxalate metabolizing activity at about 1 mM oxalate and another active microbe having a higher oxalate metabolizing activity at about 15 mM oxalate. In certain embodiments, the plurality of active microbes comprises an active microbe having a higher oxalate metabolizing activity at about 2.5 mM oxalate and another active microbe having a higher oxalate metabolizing activity at about 15 mM oxalate. In certain embodiments, the plurality of active microbes comprises an active microbe having a higher oxalate metabolizing activity at about 5 mM oxalate and another active microbe having a higher oxalate metabolizing activity at about 15 mM oxalate. In certain embodiments, the plurality of active microbes comprises an active microbe having a higher oxalate metabolizing activity at about 7.5 mM oxalate and another active microbe having a higher oxalate metabolizing activity at about 15 mM oxalate.

In certain embodiments, when tested in an in vitro oxalate metabolization assay a plurality of active microbes of the present disclosure significantly reduces the concentration of oxalate present in a sample by at least about 20%, by at least about 30%, by at least about 40%, by at least about 50%, by at least about 60%, by at least about 70%, or by at least about 80%.

In certain embodiments, a plurality of active microbes of the present disclosure significantly reduces the concentration of oxalate present in a sample of blood, serum, bile, stool, or urine when administered to a subject by at least about 20%, by at least about 30%, by at least about 40%, by at least about 50%, by at least about 60%, by at least about 70%, or by at least about 80% as compared to an untreated control subject or pre-administration levels. Concentrations of oxalate in a blood, serum, bile, stool or urine sample can be measured using a liquid chromatography-mass spectrometry (LC-MS).

### Supportive Community of Microbes

The microbial consortia of the present disclosure further comprise a supportive community of microbes that enhances one or more than one characteristic of the plurality of active microbes. For example, in certain non-limiting embodiments, the supportive community of microbes enhances gastrointestinal engraftment of the plurality of active microbes. In other embodiments, the supportive community of microbes enhances biomass of the plurality of active microbes. In other embodiments, the supportive community of microbes enhances metabolism of the first metabolic substrate by the plurality of active microbes. In other embodiments, the supportive community of microbes enhances longitudinal stability of the plurality of active microbes.

The supportive community of microbes disclosed herein metabolize one or more than one metabolite produced by the plurality of active microbes, wherein the one or more than one metabolite inhibits metabolism of the plurality of active microbes. For example, in certain non-limiting embodiments, the supportive community of microbes metabolizes formate produced by the plurality of active microbes, wherein the presence of formate inhibits the metabolism of oxalate by the plurality of active microbes. In certain embodiments, the supportive community of microbes of the current disclosure catalyzes the fermentation of polysaccharides to one or more than one of the group consisting of acetate, acetoin, 2-oxoglutarate, propionate, 1,3-propanediol, succinate, ethanol, lactate, butyrate, 2,3-butanediol, acetone, butanol, formate, H₂, and CO₂. In certain embodiments, the supportive community of microbes catalyzes the fermentation of amino acids to one or more than one of the group consisting of acetate, propionate, butanoate, butyrate, isobutyrate, 2-methylbutyrate, isovalerate, isocaproate, 3-phenylpropanoate, phloretate, 3-(1H-indol-3-yl)propanoate, 5-aminopentanoate, H₂, H₂S, and CO₂, In certain embodiments, the supportive community catalyzes the synthesis of one or more than one of the group consisting of methane from H₂ and CO₂, methane from formate and H₂, acetate from H₂ and CO₂, acetate from formate and H₂, acetate and sulfide from H₂, CO₂, and sulfate, propionate and CO₂ from succinate, succinate from H₂ and fumarate; synthesis of succinate from formate and fumarate, and butyrate, acetate, H₂, and CO₂ from lactate. In certain embodiments, the supportive community of microbes of the current disclosure catalyzes the deconjugation of conjugated bile acids to produce primary bile acids, the conversion of cholic acid (CA) to 7-oxocholic acid, the conversion of 7-oxocholic acid to 7-beta-cholic acid (7betaCA), the conversion of chenodeoxycholic acid (CDCA) to 7-oxochenodeoxycholic acid, and/or the conversion of 7-oxochenodeoxycholic acid to ursodeoxycholic acid (UDCA).

### Consortia Design

In certain embodiments, microbial consortia disclosed herein are designed to meet one or more than one of the following criteria:

(i) an ability to eliminate or reduce levels of a first metabolic substrate causing or contributing to a disease in an animal;

(ii) an ability to metabolize or convert one or more than one metabolite produced by the metabolism of the first metabolic substrate;

(iii) an ability to metabolize one or more than one nutrient typically found in the human diet;

(iv) an ability to fulfill unique and potentially beneficial biological functions in the gastrointestinal (GI) tract (e.g., bile salt hydrolase activity or butyrate production);

(v) an ability to engraft in various biological niches and physical and metabolic compartments of the GI tract of an animal;

(vi) an ability to increase biomass upon engraftment in the GI tract;

(vii) an ability to have longitudinal stability in the GI tract of an animal;

(viii) an ability to increase the flux of a precursor of the first metabolic substrate into a biochemical pathway that converts said precursor into a metabolite that is not the first metabolic substrate;

(ix) diversity of component microbial species across one or more than one taxonomic phyla; and

(x) natural prevalence of component microbial species in the GI tract of healthy adults.

In certain embodiments, the microbial consortia of the present disclosure are designed to comprise a plurality of active microbes capable of metabolizing a first metabolic substrate that causes or contributes to disease in an animal. In certain embodiments, the first metabolic substrate may be selected from, but not limited to, oxalate and a bile acid (e.g., lithocholic acid (LCA), deoxycholic acid (DCA)). In certain embodiments, the microbial consortium is designed to be capable of metabolizing the first metabolic substrate across a variety of pH ranges found within the GI tract (e.g., pH 4 to 8). In certain embodiments, the microbial consortium is designed to be capable of metabolizing the first metabolic substrate in the presence of various concentrations of first metabolic substrate as they exist in different regions of the GI tract.

In certain embodiments, the Consortia is FB-001 (Table 22) or a functional equivalent thereof. In certain embodiments, FB-001 is defined by its function. In certain embodiments, FB-001 is defined by its function as set forth in Tables 23 and/or 24. In certain embodiments, FB-001 is defined by its function as set forth in Tables 23 and 24. In certain embodiments, FB-001 is defined by its function as set forth in Table 23 or 24. In certain embodiments, FB-001 is defined by its function as set forth in Tables 34, 35, and 36. In certain embodiments, FB-001 is defined by its function as set forth in one or more of Tables 34, 35, and 36. In certain embodiments, FB-001 is defined by its function as set forth in Tables 23, 24, 34, 35, and 36. In certain embodiments, FB-001 is defined by its function as set forth in one or more of Tables 23, 24, 34, 35, and 36. In certain embodiments, methods for determining function of FB-001 are provided in Examples 6 and 7.

### Methods of Preparation

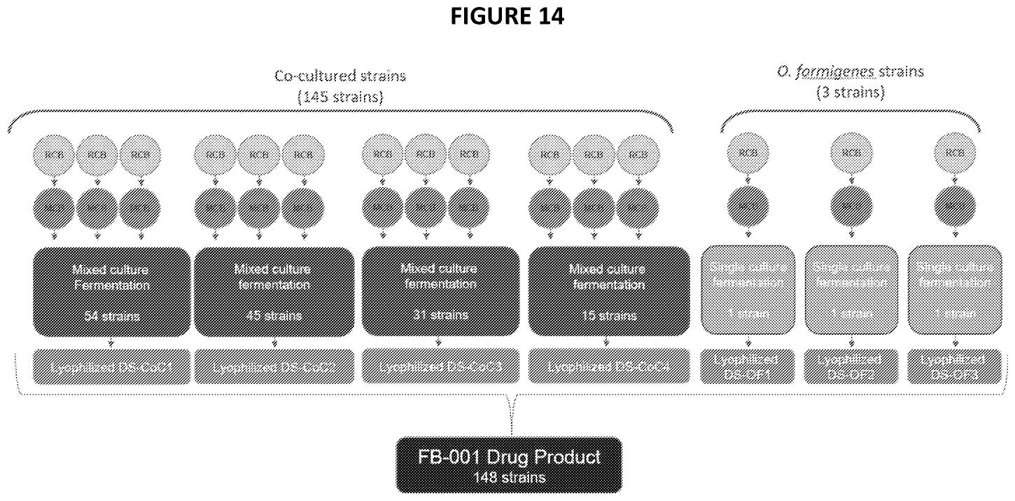

The present disclosure also provides methods for preparing and/or manufacturing the microbial consortia described herein. FIGS. 14-16 illustrate certain methods for the preparation and manufacturing of the microbial consortia described herein.

In certain embodiments, the methods comprise obtaining a donor stool and preparing a stool dilution. In certain embodiments, the stool dilution is plated onto an agar plate. In certain embodiments, the agar plate includes an anaerobic media. In certain embodiments, the agar plate includes colonies. Characterization and quality analysis of these colonies can be performed. For example, but without any limitation, 16s RNA and/or MALDI mass spectrometry could be performed. In certain embodiments, the characterized colonies can be further expanded in a broth culture. After growth and expansion, the microbes can be stored in vials for further use.

In certain embodiments, the microbes can be further expanded in a bioreactor including a cell culture medium. In certain embodiments, the cell culture medium can include:

a) soytone, D-cellobiose, yeast extract, dextrose (glucose), maltose monohydrate, magnesium sulfate heptahydrate, calcium chloride dihydrate, potassium phosphate monobasic, potassium phosphate dibasic, sodium chloride, sodium bicarbonate, volatile fatty acid solution, L-cysteine HCl monohydrate, hemin solution, vitamin solution, or a combination thereof, or

b) soytone, D-cellobiose, yeast extract, dextrose (glucose), maltose monohydrate, magnesium sulfate heptahydrate, calcium chloride dihydrate, potassium phosphate monobasic, potassium phosphate dibasic, sodium chloride, ammonium sulfate, sodium bicarbonate, volatile fatty acid solution, L-cysteine HCl monohydrate, hemin solution, vitamin solution, or a combination thereof.

In certain embodiments, the cell culture medium is YCFAC. In certain embodiments, the cell culture medium further comprises threonine.

In certain embodiments, the microbes can be expanded in a bioreactor in anaerobic conditions. In certain embodiments, the microbes can be expanded in a bioreactor in the presence of gas overlay. In certain embodiments, the microbes can be expanded in a bioreactor in absence of gas sparing.

In certain embodiments, the methods include expanding microbes in mixed cultures.

In certain embodiments, the methods comprise expanding microbes in a first mixed culture or composition comprising:

a) Clostridium citroniae, Bacteroides salyersiae, Blautia obeum, Parabacteroides merdae, Parabacteroides distasonis, Anaerostipes hadrus, Lachnospiraceae sp. FBI00033, Eubacterium eligens, Bifidobacterium dentium, Blautia wexlerae, Fusicatenibacter saccharivorans, Bacteroides nordii, Dorea formicigenerans, Dorea longicatena, Bacteroides stercorirosoris, Bifidobacterium longum, Bacteroides kribbi, Lachnospiraceae sp. FBI00071, Bacteroides thetaiotaomicron, Clostridium clostridioforme, Clostridium scindens, Roseburia hominis, Clostridium fessum, Coprococcus comes, Blautia faecis, Hungatella hathewayi, Bacteroides stercoris, Collinsella aerofaciens, Hungatella effluvii, Bifidobacterium adolescentis, Bifidobacterium catenulatum, Lactobacillus rogosae, Bacteroides faecis, Bacteroides finegoldii, Clostridiaceae sp. FBI00191, Ruminococcus faecis, Lachnoclostridium pacaense, Clostridium bolteae, Longicatena caecimuris, Eggerthella lenta, Blautia massiliensis, Bacteroides xylanisolvens, Bacteroides vulgatus, Megasphaera massiliensis, Butyricimonas faecihominis, Eisenbergiella tayi, Acidaminococcus intestini, Emergencia timonensis, Bifidobacterium pseudocatenulatum, Eubacterium hallii, Anaerofustis stercorihominis, Eubacterium ventriosum, Blautia hydrogenotrophica, and Lachnospiraceae sp. FBI00290, or a functional equivalent thereof, or

b) FBI00001, FBI00002, FBI00010, FBI00013, FBI00029, FBI00032, FBI00033, FBI00034, FBI00043, FBI00044, FBI00048, FBI00050, FBI00051, FBI00057, FBI00059, FBI00060, FBI00070, FBI00071, FBI00076, FBI00079, FBI00087, FBI00093, FBI00102, FBI00109, FBI00117, FBI00120, FBI00125, FBI00127, FBI00128, FBI00145, FBI00162, FBI00174, FBI00184, FBI00190, FBI00191, FBI00194, FBI00198, FBI00199, FBI00200, FBI00201, FBI00205, FBI00206, FBI00211, FBI00220, FBI00221, FBI00236, FBI00245, FBI00248, FBI00251, FBI00254, FBI00267, FBI00278, FBI00288, and FBI00290, or a functional equivalent thereof.

In certain embodiments, the methods comprise expanding microbes in a second mixed culture or composition comprising:

a) Acutalibacter timonensis, Alistipes onderdonkii, Bacteroides uniformis, Eubacterium rectale, Alistipes timonensis, Bacteroides kribbi, Coprococcus eutactus, Bilophila wadsworthia, Bacteroides caccae, Alistipes shahii, Parasutterella excrementihominis, Paraprevotella clara, Sutterella wadsworthensis, Sutterella massiliensis, Porphyromonas asaccharolytica, Ruminococcus bromii, Monoglobus pectinilyticus, Ruminococcaceae sp. FBI00097, Gordonibacter pamelaeae, Bacteroides uniformis, Gordonibacter pamelaeae, Bacteroides fragilis, Phascolarctobacterium faecium, Monoglobus pectinilyticus, Clostridium aldenense, Ruthenibacterium lactatiformans, Bacteroides ovatus, Bifidobacterium bifidum, Anaerotruncus massiliensis, Clostridium aldenense, Sutterella wadsworthensis, Catabacter hongkongensis, Alistipes senegalensis, Ruminococcaceae sp. FBI00233, Alistipes shahii, Dielma fastidiosa, Eubacterium siraeum, Faecalibacterium prausnitzii, Turicibacter sanguinis, Eubacterium rectale, Bacteroides caccae, Methanobrevibacter smithii, Barnesiella intestinihominis, Alistipes onderdonkii, and Methanobrevibacter smithii, or a functional equivalent thereof, or

b) FBI00004, FBI00012, FBI00015, FBI00018, FBI00019, FBI00021, FBI00038, FBI00040, FBI00046, FBI00061, FBI00066, FBI00075, FBI00077, FBI00080, FBI00081, FBI00085, FBI00092, FBI00097, FBI00099, FBI00112, FBI00132, FBI00137, FBI00140, FBI00149, FBI00151, FBI00176, FBI00189, FBI00197, FBI00208, FBI00212, FBI00224, FBI00226, FBI00229, FBI00233, FBI00235, FBI00237, FBI00243, FBI00244, FBI00258, FBI00260, FBI00263, FBI00270, FBI00273, FBI00277, and FBI00292, or a functional equivalent thereof.

In certain embodiments, the methods comprise expanding microbes in a third mixed culture or composition comprising:

a) Bifidobacterium adolescentis, Bifidobacterium longum, Bifidobacterium pseudocatenulatum, Bacteroides thetaiotaomicron, Coprococcus comes, Fusicatenibacter saccharivorans, Eggerthella lenta, Eubacterium eligens, Bacteroides xylanisolvens, Lactobacillus rogosae, Clostridium citroniae, Collinsella aerofaciens, Blautia obeum, Eggerthella lenta, Blautia wexlerae, Lachnoclostridium pacaense, Bacteroides vulgatus, Parabacteroides merdae, Dorea formicigenerans, Ruminococcus faecis, Roseburia hominis, Anaerostipes hadrus, Bifidobacterium adolescentis, Bifidobacterium pseudocatenulatum, Clostridium bolteae, Eisenbergiella tayi, Dorea longicatena, Eggerthella lenta, Bacteroides stercoris, Hungatella hathewayi, and Bacteroides xylanisolvens, or a functional equivalent thereof, or

b) FBI00009, FBI00011, FBI00016, FBI00020, FBI00025, FBI00027, FBI00030, FBI00047, FBI00052, FBI00053, FBI00056, FBI00062, FBI00078, FBI00096, FBI00104, FBI00110, FBI00111, FBI00113, FBI00115, FBI00116, FBI00123, FBI00124, FBI00126, FBI00135, FBI00147, FBI00159, FBI00167, FBI00170, FBI00232, FBI00255, and FBI00271, or a functional equivalent thereof.

In certain embodiments, the methods comprise expanding microbes in a fourth mixed culture or composition comprising:

a) Alistipes putredinis, Dialister succinatiphilus, Akkermansia muciniphila, Ruminococcus bromii, Dialister invisus, Bacteroides massiliensis, Bilophila wadsworthia, Holdemanella biformis, Parasutterella excrementihominis, Alistipes sp. FBI00180, Bacteroides coprocola, Alistipes sp. FBI00238, Alistipes putredinis, Eubacterium xylanophilum, and Senegalimassilia anaerobia, or a functional equivalent thereof, or

b) FBI00022, FBI00049, FBI00068, FBI00069, FBI00152, FBI00165, FBI00171, FBI00175, FBI00177, FBI00180, FBI00182, FBI00238, FBI00269, FBI00274, and FBI00281, or a functional equivalent thereof.

In certain embodiments, the methods include expanding microbes in single cultures.

In certain embodiments, the methods comprise expanding microbes in a first single culture (or fifth composition) comprising a) a first O. formigenes strain; or b) FBI00067 or a functional equivalent thereof.

In certain embodiments, the methods comprise expanding microbes in a second single culture (or sixth composition) comprising a) a second O. formigenes strain; or b) FBI00133 or a functional equivalent thereof.

In certain embodiments, the methods comprise expanding microbes in a third single culture (or seventh composition) comprising a) a third O. formigenes strain; or b) FBI00289 or a functional equivalent thereof.

In certain embodiments, the methods comprise lyophilizing cultures and compositions described herein. In certain embodiments, the cultures and compositions comprises a lyoprotectant. In certain embodiments, the lyoprotectant comprises maltodextrin. In certain embodiments, the lyoprotectant comprises inulin. In certain embodiments, the lyoprotectant comprises maltodextrin and inulin. In certain embodiments, the maltodextrin is present at a concentration of about 8%. In certain embodiments, the inulin is present at a concentration of about 0.5%.

In certain embodiments, the methods comprise blending and/or mixing lyophilized cultures and compositions outlined above. Additional information on the strains for each composition can be found in Table 22.

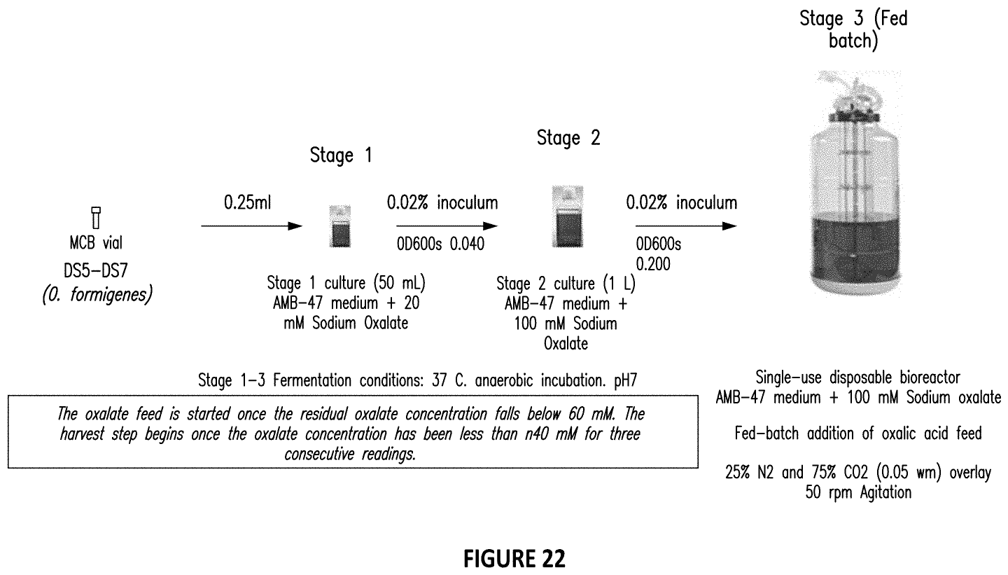

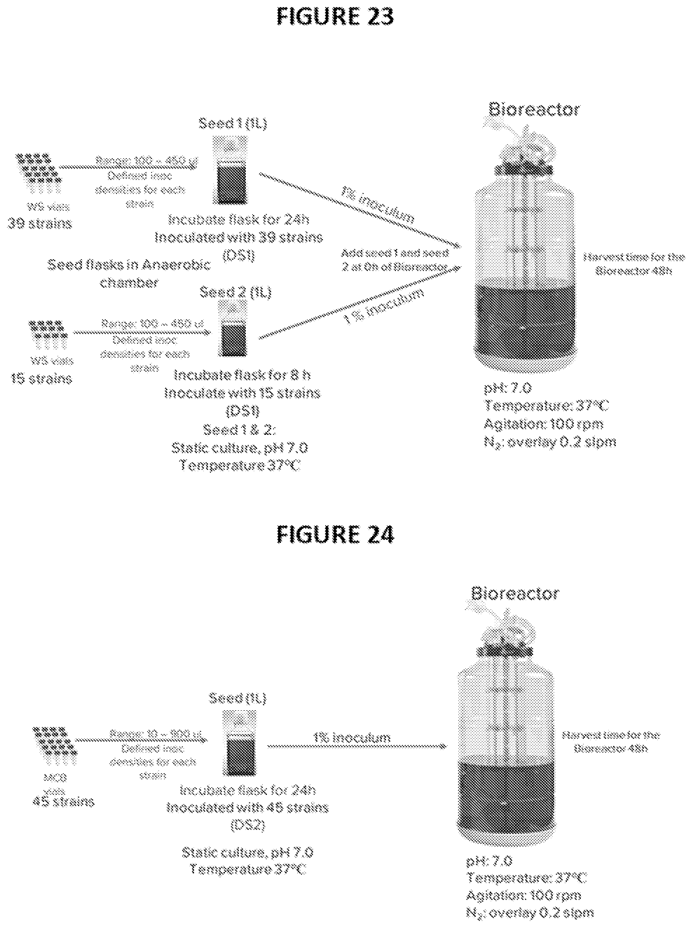

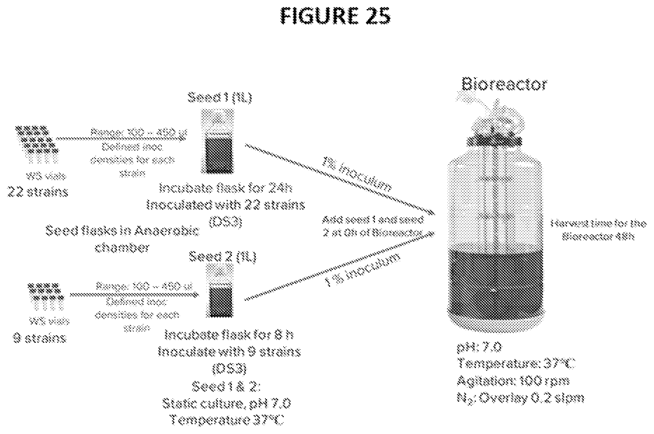

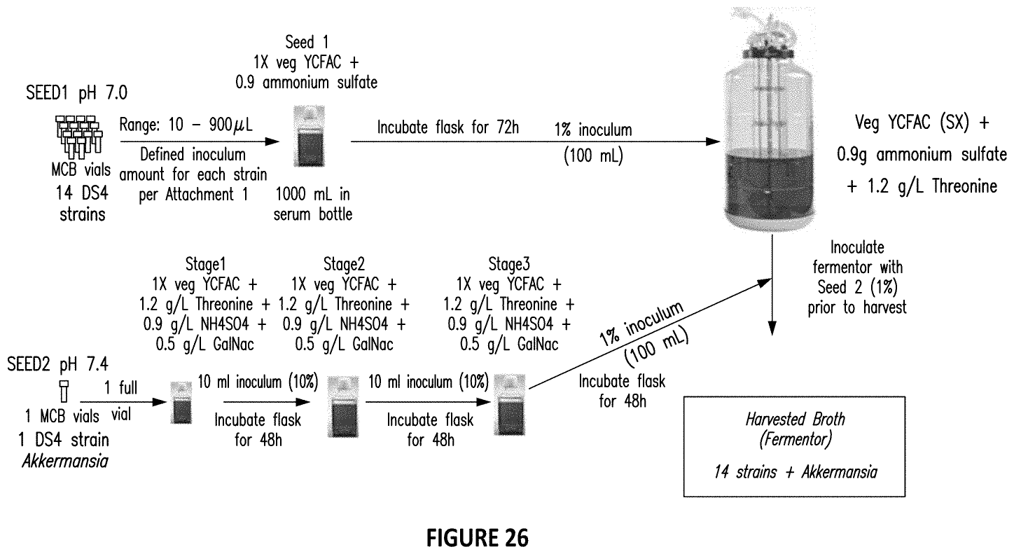

In certain embodiments, DS1 as described in Table 22 is prepared using the method described in FIG. 23. In certain embodiments, DS2 as described in Table 22 is prepared using the method described in FIG. 24. In certain embodiments, DS3 as described in Table 22 is prepared using the method described in FIG. 25. In certain embodiments, DS4 as described in Table 22 is prepared using the method described in FIG. 26. In certain embodiments, DS5-DS7 (i.e., the manufacture of O. formigenes) as described in Table 22 are prepared using the method described in FIG. 22. In certain embodiments, the manufacture of FB-001 comprises the separate manufacture of each of DS1-DS7 as described in FIGS. 22-26, followed by blending to achieve a uniform distribution of each of the DSs. In certain embodiments, the blending of DS1-DS7 is followed by encapsulation for oral administration.

### Pharmaceutical Compositions

The present disclosure also provides pharmaceutical compositions that contain an effective amount of a microbial consortium described herein. The composition can be formulated for use in a variety of delivery systems. One or more physiologically acceptable buffer(s) or carrier(s) can also be included in the composition for proper formulation. Suitable formulations for use in the present disclosure are found in Remington's Pharmaceutical Sciences, Mack Publishing Company, Philadelphia, Pa., 17th ed., 1985. For a brief review of methods for drug delivery, see, e.g., Langer (Science 249:1527-1533, 1990).

In certain embodiments, microbial cells of the present disclosure are harvested by microfiltration and centrifugation. In certain embodiments, microfiltration is done with a membrane comprising a nonreactive polymer. For example, in certain non-limiting embodiments, said membrane comprises Polyvinylidene fluoride, Polysulfones, or nitrocellulose. In certain embodiments, a membrane for microfiltration has a pore size of approximately 0.2 to 0.45 μm. In certain embodiments, the cells are centrifuged at approximately 1000 to 30000, 5000 to 30000, 10000 to 30000, 15000 to 30000, 20000 to 30000, 25000 to 30000, 1000 to 25000, 5000 to 25000, 10000 to 25000, 15000 to 25000, 20000 to 25000, 1000 to 20000, 5000 to 20000, 10000 to 20000, 15000 to 20000, 1000 to 15000, 5000 to 15000, 10000 to 15000, 1000 to 10000, 5000 to 10000, 1000 to 5000 g force. In certain embodiments, the cells are concentrated to approximately 1×10⁶ CFUs per milliliter to 1×10¹² CFUs per milliliter, 1×10⁷ CFUs per milliliter to 1×10¹² CFUs per milliliter, 1×10⁸ CFUs per milliliter to 1×10¹² CFUs per milliliter, 1×10⁹ CFUs per milliliter to 1×10¹² CFUs per milliliter, 1×10¹⁰ CFUs per milliliter to 1×10¹² CFUs per milliliter, 1×10¹¹ CFUs per milliliter to 1×10¹² CFUs per milliliter, 1×10⁶ CFUs per milliliter to 1×10¹¹ CFUs per milliliter, 1×10⁷ CFUs per milliliter to 1×10¹¹ CFUs per milliliter, 1×10⁸ CFUs per milliliter to 1×10¹¹ CFUs per milliliter, 1×10⁹ CFUs per milliliter to 1×10¹¹ CFUs per milliliter, 1×10¹⁰ CFUs per milliliter to 1×10¹¹ CFUs per milliliter, 1×10⁶ CFUs per milliliter to 1×10¹⁰ CFUs per milliliter, 1×10⁷ CFUs per milliliter to 1×10¹⁰ CFUs per milliliter, 1×10⁸ CFUs per milliliter to 1×10¹⁰ CFUs per milliliter, 1×10⁹ CFUs per milliliter to 1×10¹⁰ CFUs per milliliter, 1×10⁶ CFUs per milliliter to 1×10⁹ CFUs per milliliter, 1×10⁷ CFUs per milliliter to 1×10⁹ CFUs per milliliter, 1×10⁸ CFUs per milliliter to 1×10⁹ CFUs per milliliter, 1×10⁶ CFUs per milliliter to 1×10⁸ CFUs per milliliter, 1×10⁷ CFUs per milliliter to 1×10⁸ CFUs per milliliter, or 1×10⁶ CFUs per milliliter to 1×10⁷ CFUs per milliliter.

In certain embodiments, microbial cells of the present disclosure are frozen. In certain embodiments, the microbial cells of the present disclosure are mixed with one or more cryoprotective agents (CPAs) before freezing. In certain embodiments, the ratio of cells to CPA is approximately 25:1, 10:1, 5:1, 4:1, 3:1, 2:1, 1:1, 1:2, 1:3, 1:4, 1:5, 1:10, or 1:25. In certain embodiments, a CPA comprises one or more of glycerol, maltodextrin, sucrose, inulin, trehalose, and alginate. In certain embodiments, a CPA further comprises one or more antioxidants. In certain embodiments, an antioxidant is selected from the list of cysteine, ascorbic acid, and riboflavin.

In certain embodiments, the microbial cells of the present disclosure are lyophilized. In certain embodiments, the lyophilized cells are used to make an orally-administered dose of the disclosure. In certain embodiments, primary drying is conducted below approximately −20° C. In certain embodiments, primary drying is followed by a secondary drying at a higher temperature, e.g. greater than 0° C., greater than 5° C., or greater than 10° C.

### Functionally Equivalent and Identical Drug Products to FB-001

The strains included in FB-001 are described herein by 16S RNA sequences and functional characteristics. Based on this, equivalent Consortia to FB-001 can be generated by screening multiple of the same strain to find equivalent strains with equivalent function to those that comprise FB-001. Accordingly, identical strains may theoretically have different functions, strains can be screened using 16S RNA and Biolog as described herein to identify functionally identical and equivalent strains from any fecal collection using the methods of collection described herein.

It is important to note that FB-001 was articulately designed to have multiple of the same strain in the Consortia. The reason for this to have redundancy to ensure function; however, such redundancy is not required for equivalent function so long as one of the otherwise redundant strains is included in the final drug product at a sufficient viable cell count amount to achieve in vivo function in a subject. Accordingly, a Consortia that is equivalent or identical to FB-001 may contain all redundancies (see Table 22) or alternatively may contain no or fewer redundancies per strain so long as the included strains achieve in vivo function in a subject.

In an alternative approach to creating a functionally equivalent Consortia to FB-001, one of skill in the art could recreate a consortia of supportive microbes from healthy fecal donors and supplement the supportive microbes with one or more O. formigenes strains. In certain embodiments, the supportive microbes will be supplemented with two or more O. formigenes strains or specifically three O. formigenes strains. The supportive microbes may comprise anywhere between 10 and 200 microbes so long as such supportive community supports and encourages the growth, health, and engraftment of the O. formigenes strain(s) in a subject. FB-001 was designed to have 148 microbes to mimic a complete, healthy microbiome. Accordingly, equivalent Consortia may comprise approximately 148 microbes, including O. formigenes strain(s). However, it is interesting to note that older subjects often have smaller microbiomes; accordingly, a functionally equivalent Consortia to FB-001 may also have far fewer microbes (e.g., 30-40, 40-50, 50-60, 60-70, 70-80, 8-90, 90-100, 100-110, 110-120, 120-130, 130-140, or 140-150 microbes, including O. formigenes strain(s)).

### Therapeutic Applications

The present disclosure provides Consortia capable of engrafting into one or more than one niche of a gastrointestinal tract where it is capable of metabolizing a first metabolic substrate that causes or contributes to disease in an animal. In certain embodiments, the animal is a human.

In certain embodiments of the disclosure, when administered to an animal, the animal is pre-treated with one or more antibiotics prior to administration of the Consortium. In certain embodiments, the one or more antibiotics is selected from ampicillin, enrofloxacin, clarithromycin, and metronidazole. In certain embodiments, the animal is pre-treated with a polyethylene glycol bowel-preparation procedure.

In certain embodiments, when administered to an animal, the Consortia significantly reduces the concentration of a first metabolic substrate present in the blood, serum, bile, stool or urine as compared to samples collected pretreatment from the same animal or from corresponding control animal that have not been administered with the microbial consortium.

In certain embodiments, a Consortia is used to treat a subject having or at risk of developing a metabolic disease or condition. In certain embodiments, the metabolic disease is primary hyperoxaluria. In certain embodiments, the metabolic disease is secondary hyperoxaluria. In certain embodiments, the metabolic disease is enteric hyperoxaluria. In certain embodiments, the metabolic disease is secondary hyperoxaluria associated with bowel resection surgery or IBD. In certain embodiments, a Consortium significantly reduces the concentration of oxalate present in a sample of blood, serum, bile, stool, or urine when administered to a subject by at least about 20%, by at least about 30%, by at least about 40%, by at least about 50%, by at least about 60%, by at least about 70%, or by at least about 80% as compared to untreated subjects or pre-administration concentrations.

In certain embodiments, a Consortia significantly alters the profile and/or concentration of bile acids present in an animal. For example, in certain non-limiting embodiments, a Consortia significantly alters the profile and/or concentration of Tβ-MCA, Tα-MCA, TUDCA, THDCA, TCA, 7β-CA, 7-oxo-CA, TCDCA, Tω-MCA, TDCA, α-MCA, β-MCA, ω-MCA, Muro-CA, d4-CA, CA, TLCA, UDCA, HDCA, CDCA, DCA, and LCA in an animal.

In certain embodiments, a high-complexity defined gut microbial community of the present disclosure can be used to treat an animal having a cholestatic disease, such as, for example, primary sclerosing cholangitis, primary biliary cholangitis, progressive familial intrahepatic cholestasis, or nonalcoholic steatohepatitis. For example, in certain non-limiting embodiments, the animal may be a mammal, and more particularly a human.

In certain embodiments, a Consortia can be administered via an enteric route. For example, in certain non-limiting embodiments, a microbial consortium is administered orally, rectally (e.g., by enema, suppository, or colonoscope), or by oral or nasal tube.

In certain embodiments, a Consortia is administered orally. In certain embodiments the oral administration is by a powder. In certain embodiments the oral administration is by a slurry. In certain embodiments the oral administration is by pills or capsules.

In certain embodiments, a Consortia can be administered to a specific location along the gastrointestinal tract. For example, in certain non-limiting embodiments, a microbial consortium can be administered into one or more than one gastrointestinal location including the mouth, esophagus, stomach, small intestine (duodenum, jejunum, ileum), large intestine (cecum, ascending colon, transverse colon, descending colon), or rectum. In certain embodiments, a microbial consortium can be administered in all regions of the gastrointestinal tract.

### Methods of Treating Hyperoxaluria

In certain embodiments, a Consortia is used to treat hyperoxaluria. Hyperoxaluria is a metabolic disorder characterized by a significant increase in urinary oxalate (UOx) excretion (>40 mg/24 h) that can lead to the formation of kidney stones and ultimately kidney damage. It is either due to a genetic defect that results in overproduction of oxalate by the liver (primary) or from absorption of too much oxalate from the diet (secondary). Secondary hyperoxaluria is further characterized as either dietary, due to excessive intake of oxalate or its precursors, or enteric hyperoxaluria (EH). Enteric hyperoxaluria is a complex medical condition characterized by excess absorption of dietary oxalate, usually caused by malabsorption of fat, for example after gastric bypass surgery, or an increased permeability of the gut for oxalate due to underlying gastrointestinal diseases. Twenty-four-hour UOx excretion is an established biomarker of disease that is routinely measured in clinical practice to diagnose and manage patients at risk for EH and calcium oxalate kidney stones. While an increase in UOx increases the risk for kidney stone events, it is believed that a decrease of 20% or more will reduce the incidence of kidney stones by 25% or more. The increase in UOx excretion (>40 mg/24 h) that characterizes EH occurs because non-absorbed fatty acids bind to calcium in the small intestine, thereby making it unavailable to precipitate oxalate. Soluble oxalate consequently builds up to a relatively high concentration in the lumen and can diffuse passively out of the colon into the blood for excretion in the urine. Calcium oxalate crystals can precipitate within kidney tubules, bind to epithelial cells, and cause obstruction. Attached crystals can be phagocytosed and transcytosed into the kidney interstitium, thereby releasing inflammatory mediators that can contribute to oxalate nephropathy and potentially progressive loss of kidney function.

In certain embodiments, while the presentation of hyperoxaluria can be variable, the first clinical manifestation is often the occurrence of a kidney stone (nephrolithiasis), which can be extremely painful and debilitating and sometimes requires surgical removal. As oxalate can complex with calcium to form insoluble crystals, chronically elevated UOx levels are a major risk factor for the development of kidney stones and ultimately kidney damage. Regardless of the frequency of kidney stones, oxalate nephropathy in patients with severe hyperoxaluria can lead to progressive kidney deterioration, chronic kidney disease (CKD) and eventually end stage renal disease (ESRD) which can be fatal.

The prevalence of EH has increased in recent years affecting over 250,000 Americans. Of the 250,000 patients with EH in the US in 2019, approximately 60% were a result of RYGB surgery for the treatment of obesity. As the global prevalence of obesity has increased in recent years, bariatric surgery procedures, RYGB in particular, have emerged as a widely used procedure to treat obesity. While the RYGB procedure can be advantageous for patients, including increasing life expectancy and reducing the risk of obesity related cancers, it can also lead to EH within 6 to 24 months of surgery, which can then progress to kidney stones and, in severe cases, kidney damage. A 36.4% increase in UOx was identified as a key lithogenic risk factor after RYGB in an analysis of seven studies including 277 patients before and after RYGB. Additionally, plasma oxalate and urine calcium oxalate supersaturation were found to be significantly increased compared with presurgical levels at 6 and 12 months following RYGB. Collectively, biomarkers such as urinary and plasma oxalate as well as calcium oxalate supersaturation are excellent prognostic indicators of EH, kidney stone formation and kidney damage and reduction of these markers may lead to improved outcomes.

There are currently no approved therapies for the reduction of UOx excretion in patients with EH. The management or standard of care options for patients with EH are limited to high fluid intake to increase urine output, correcting the underlying GI disease to reduce fat malabsorption, intensive dietary modifications to reduce intake of oxalate, and the use of calcium salts to bind oxalate in the GI tract. Compliance with these strategies tends to be low and many patients continue to experience hyperoxaluria with recurrent kidney stones and are at continued risk for long-term significant, irreversible, and progressive kidney damage.

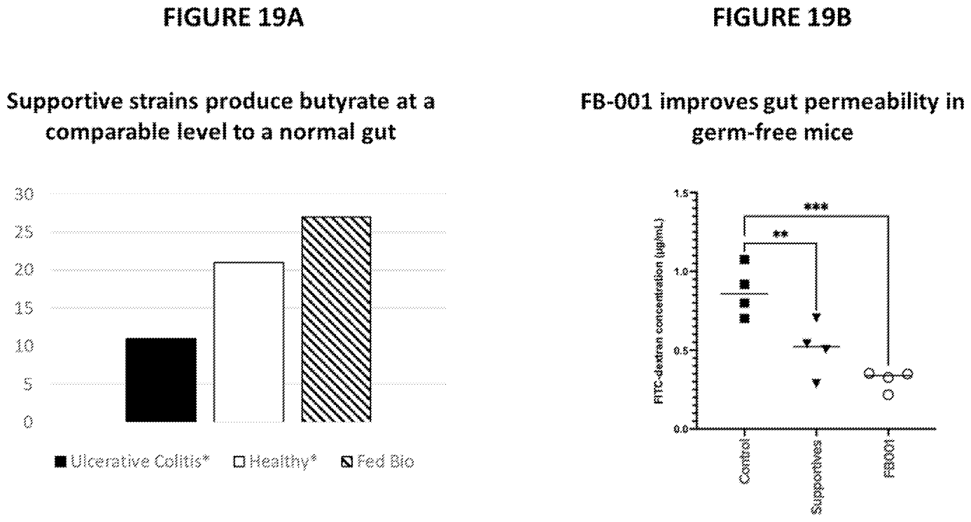

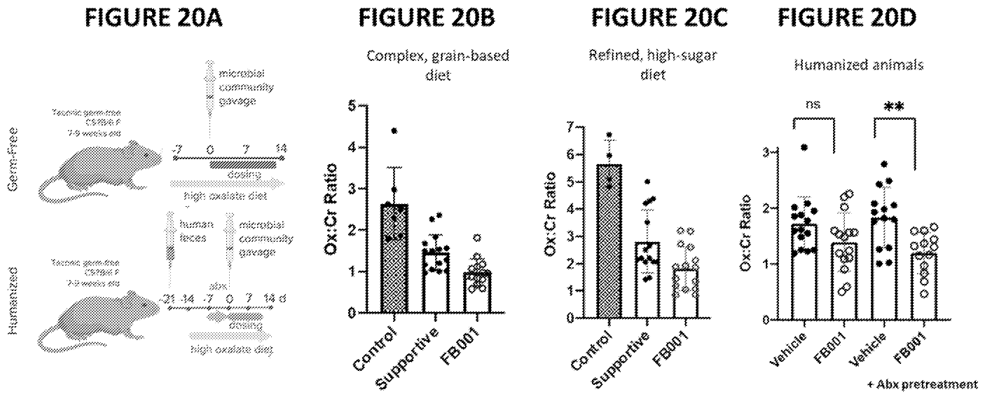

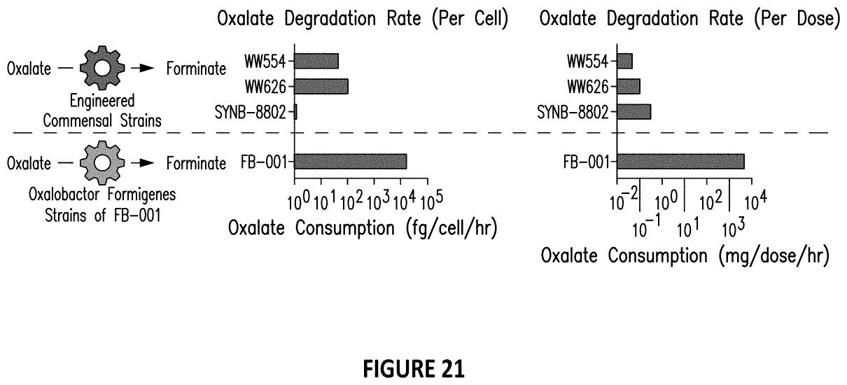

In certain embodiments, the Consortia described herein comprise one or more O. formigenes strain(s) and can be administered to subjects for the treatment of enteric hyperoxaluria. In certain embodiments, the Consortia described herein comprise one or more O. formigenes strain(s) and can be administered to subjects for the treatment of hyperoxaluria. In certain embodiments, the Consortia described herein comprise one or more O. formigenes strain(s) and can be administered to subjects for the treatment of primary hyperoxaluria. In certain embodiments, the Consortia described herein comprise one or more O. formigenes strain(s) and can be administered to subjects for the treatment of secondary hyperoxaluria. In certain embodiments, the FB-001 can be administered to subjects for the treatment of enteric hyperoxaluria. In certain embodiments, the FB-001 can be administered to subjects for the treatment of hyperoxaluria. In certain embodiments, the FB-001 can be administered to subjects for the treatment of primary hyperoxaluria. In certain embodiments, the FB-001 can be administered to subjects for the treatment of secondary hyperoxaluria. In certain embodiments, the treatment of hyperoxaluria by FB-001 or a functionally equivalent Consortia thereof comprises the reduction of gut permeability (FIG. 19). In certain embodiments, the treatment of hyperoxaluria by FB-001 or a functionally equivalent Consortia thereof comprises the increased production or production equivalent to a normal, healthy gut of SCFAs (FIG. 20). In certain embodiments, the treatment of hyperoxaluria by FB-001 or a functionally equivalent Consortia thereof comprises the reduction of urinary oxalate independent of diet (FIGS. 20A-20D). In certain embodiments, the treatment of hyperoxaluria by FB-001 or a functionally equivalent Consortia thereof comprises oxalate degradation (FIG. 21). In certain embodiments, the treatment of hyperoxaluria by FB-001 or a functionally equivalent Consortia thereof comprises oxalate degradation at a rate of at least 10³ fg/cell/hr oxalate consumption (FIG. 21). In certain embodiments, the treatment of hyperoxaluria by FB-001 or a functionally equivalent Consortia thereof comprises oxalate degradation at a rate of at least 10² fg/cell/hr oxalate consumption (FIG. 21). In certain embodiments, the treatment of hyperoxaluria by FB-001 or a functionally equivalent Consortia thereof comprises oxalate degradation at a rate of at least 10⁴ fg/cell/hr oxalate consumption (FIG. 21). In certain embodiments, the treatment of hyperoxaluria by FB-001 or a functionally equivalent Consortia thereof comprises oxalate degradation at a rate of at least 103 mg/dose/hr oxalate consumption (FIG. 21). In certain embodiments, the treatment of hyperoxaluria by FB-001 or a functionally equivalent Consortia thereof comprises oxalate degradation at a rate of at least 10¹ mg/dose/hr oxalate consumption (FIG. 21). In certain embodiments, the treatment of hyperoxaluria by FB-001 or a functionally equivalent Consortia thereof comprises oxalate degradation at a rate of at least 102 mg/dose/hr oxalate consumption (FIG. 21). In certain embodiments, the treatment of hyperoxaluria by FB-001 or a functionally equivalent Consortia thereof comprises oxalate degradation at a rate of greater than 10³ mg/dose/hr oxalate consumption (FIG. 21). In certain embodiments, the treatment of hyperoxaluria by FB-001 or a functionally equivalent Consortia thereof comprises oxalate degradation at a rate of at least 10⁻¹ mg/dose/hr oxalate consumption (FIG. 21).

### Dosages

In certain embodiments, a Consortia is administered as a single dose or as multiple doses. In certain embodiments, a Consortia is administered once a day for 2 days, 3 days, 4 days, 5 days, 6 days, 1 week, 2 weeks 3 weeks, 1 month, 2 months, 3 months, 4 months, 5 months, 6 months, or 1 year. In certain embodiments, a Consortia is administered multiple times daily. In certain embodiments, a Consortia is administered twice daily, three times daily, 4 times daily, or 5 times daily. In certain embodiments, a Consortia is administered intermittently. In certain embodiments, a Consortia is administered once weekly, once monthly, or when a subject is in need thereof.

In certain embodiments, a Consortia is administered at an effective dose to allow for engraftment and substrate metabolism. In certain embodiments, a Consortia is administered at an effective dose to allow for engraftment and oxalate metabolism. In certain embodiments, a Consortia is administered at an effective dose to allow for engraftment and urinary oxalate reduction.

In certain embodiments, a Consortia is administered at a first loading dose and then followed by maintenance doses. In certain embodiments, the first loading dose is administered for 1 day, 2 days, 3 days, 4 days, 5 days, 6 days, 7 days, 8 days, 9 days, or 10 days. In certain embodiments, the loading dose is administered for 1-3 days. In certain embodiments, the loading dose is administered for 2-4 days. In certain embodiments, the loading dose is administered for 2-3 days. In certain embodiments, the loading dose is administered for 3-5 days. In certain embodiments, the loading dose is administered for 4-6 days. In certain embodiments, the loading dose is administered for 5-7 days. In certain embodiments, the loading dose is administered for 1 day. In certain embodiments, the loading dose is administered for 3 days. In certain embodiments, the loading dose is administered for 2 days. In certain embodiments, the maintenance doses are administered for 5-10 days following the last loading dose. In certain embodiments, the maintenance doses are administered for 7-12 days following the last loading dose. In certain embodiments, the maintenance doses are administered for 10-14 days following the last loading dose. In certain embodiments, the maintenance doses are administered for 14-21 days following the last loading dose. In certain embodiments, the maintenance doses are administered for 21-28 days following the last loading dose. In certain embodiments, the maintenance doses are administered for 14 days following the last loading dose. In certain embodiments, the maintenance doses are administered for 21 days following the last loading dose. In certain embodiments, the maintenance doses are administered for 28 days following the last loading dose. In certain embodiments, the maintenance doses are administered for about 8 days following the last loading dose. In certain embodiments, the maintenance doses are administered for about 7 days following the last loading dose. In certain embodiments, the maintenance doses are administered for about 6 days following the last loading dose. In certain embodiments, the maintenance doses are administered for about 9 days following the last loading dose. In certain embodiments, the maintenance doses are administered for about 10 days following the last loading dose. In certain embodiments, the loading dose is administered for 2 days and the maintenance dose is administered for 6 days (for a total of a 8 day course of treatment). In certain embodiments, the loading dose is administered for 2 days and the maintenance dose is administered for 7 days (for a total of a 9 day course of treatment). In certain embodiments, the loading dose is administered for 2 days and the maintenance dose is administered for 8 days (for a total of a 10 day course of treatment). In certain embodiments, the loading dose is administered for 9 days and the maintenance dose is administered for 9 days (for a total of a 11 day course of treatment). In certain embodiments, the loading dose is administered for 2 days and the maintenance dose is administered for 10 days (for a total of a 12 day course of treatment). In certain embodiments, the Consortia is FB-001. In certain embodiments, the loading dose follows the pretreatment with antibiotics as described in the Combination Therapy section below. In certain embodiments, the loading dose follows the pretreatment with a bowel preparation as described in the Combination Therapy section below. In certain embodiments, the loading dose follows the pretreatment with antibiotics and a bowel preparation as described in the Combination Therapy section below.

In certain embodiments, FB-001 (i.e., FB-001), is formulated by blending the seven lyophilized DSs containing the 148 microbial species and filling them into coated enteric capsules. In certain embodiments, the capsules are provided in blister packaging or alternative packaging to allow for no or low oxygen exposure (e.g., packaging to sustain the viability of anaerobic microbes). In certain embodiments, each capsule contains a range of 5×10¹⁰ to 5×10¹¹ viable cells/capsule. In certain embodiments, each capsule contains a range of 5×10⁹ to 5×10¹⁰ viable cells/capsule. In certain embodiments, each capsule contains a range of 5×10¹¹ to 5×10¹, viable cells/capsule. In certain embodiments, FB-001 is orally dosed at up to 10¹² viable cells on Days 1 and 2, and up to 10¹¹ viable cells on Days 3 to 10. In certain embodiments, maltodextrin is included as an excipient in the capsules.

In certain embodiments, the FB-001 is comprised of approximately 10-15% O. formigenes. In certain embodiments, the FB-001 is comprised of approximately 15-20% O. formigenes. In certain embodiments, the FB-001 is comprised of approximately 20-25% O. formigenes. In certain embodiments, the FB-001 is comprised of approximately 25-30% O. formigenes. In certain embodiments, the FB-001 is comprised of approximately 30-35% O. formigenes. In certain embodiments, the FB-001 is comprised of approximately 35-40% O. formigenes. In certain embodiments, the FB-001 is comprised of approximately 45-50% O. formigenes. In certain embodiments, the three strains of O. formigenes with 16S RNA sequences of SEQ ID NOs: 42, 79, and 146 are provided in approximately equal amounts. In certain embodiments, the three strains of O. formigenes with 16S RNA sequences of SEQ ID NOs: 42, 79, and 146 are provided in unequal amounts. In certain embodiments, the three strains of O. formigenes with 16S RNA sequences of SEQ ID NOs: 42, 79, and 146 are provided in similar amounts. In certain embodiments, the three strains of O. formigenes with 16S RNA sequences of SEQ ID NOs: 42, 79, and 146 are provided in equal amounts.

In certain embodiments, the total O. formigenes content of each capsule is approximately 25-35% on a relative abundance basis. In certain embodiments, the total O. formigenes content of each capsule is approximately 20%, 21%, 22%, 23%, 24% or 25% on a relative abundance basis. In certain embodiments, the total O. formigenes content of each capsule is approximately 15%, 16%, 17%, 18% or 19% on a relative abundance basis. In certain embodiments, the total O. formigenes content of each capsule is approximately 20%, 21%, 22%, 23%, 24% or 25% on a relative abundance basis. In certain embodiments, the total O. formigenes content of each capsule is approximately 30%, 31%, 32%, 33%, 34% or 35% on a relative abundance basis.

In certain embodiments, the total O. formigenes content of each capsule is approximately 32% on a relative abundance basis. In certain embodiments, this translates to a total O. formigenes content of 40% on a viable cell count basis. In certain embodiments, for the remaining strains, relative abundance values ranged from 18% to 0.015%, or three orders of magnitude. In certain embodiments, the distribution is typical of the human microbiome, which follows a power law distribution in which most species are at a low relative abundance. In certain embodiments, the absence of detection of a strain should not be interpreted as its absence from the drug substance. In certain embodiments, the 60 detected strains account for 95.932% of the biomarkers detected in FB-001 DP. In certain embodiments, the remaining 88 strains therefore account for 4.068% of the biomarkers. In certain embodiments, the relative abundance profile is expected to vary between batches and data will continue to be collected during development to understand the magnitude of the variability.

In certain embodiments, each capsule of FB-001 contains a range of 5×10¹⁰ to 5×10¹¹ viable cells/capsule with approximately 40% O. formigenes and a viable cell count basis and with relative abundance values of the remaining 145 strains ranging from 18% to 0.015%.

In certain embodiments, the dosage comprises treatment for 10 days consisting of a loading dose of 10 capsules (1×10\hat{ }12 viable cells) on Day 1 and Day 2 and a dose of 1 capsule (1×10\hat{ }11 viable cells) on Day 3 to Day 10. In certain embodiments, this dosing scheme follows pretreatment with antibiotics as described herein. In certain embodiments, the pretreatment with antibiotics comprises pretreatment with 500 mg metronidazole and 500 mg clarithromycin as described herein. In certain embodiments, this dosing scheme follows pretreatment with a bowel preparation as described herein. In certain embodiments, the bowel preparation comprises pretreatment with MiraLax. In certain embodiments, this dosing scheme follows pretreatment with antibiotics as and pretreatment with a bowel preparation as described herein.

### Combination Therapy

In certain embodiments, a Consortia can be administered in combination with other agents. In certain embodiments, a Consortia can be administered with an antimicrobial agent, an antifungal agent, an antiviral agent, an antiparasitic agent or a prebiotic. In certain embodiments, a Consortia can be administered subsequent to administration of an antimicrobial agent, an antifungal agent, an antiviral agent, an antiparasitic agent or a prebiotic. In certain embodiments, administration may be sequential over a period of hours or days, or simultaneously.

For example, in certain non-limiting embodiments, a microbial consortium can be administered with, or pre-administered with, one or more than one antibacterial agent selected from fluoroquinolone antibiotics (ciprofloxacin, Levaquin, floxin, tequin, avelox, and norflox); cephalosporin antibiotics (cephalexin, cefuroxime, cefadroxil, cefazolin, cephalothin, cefaclor, cefamandole, cefoxitin, cefprozil, and ceftobiprole); penicillin antibiotics (amoxicillin, ampicillin, penicillin V, dicloxacillin, carbenicillin, vancomycin, and methicillin); tetracycline antibiotics (tetracycline, minocycline, oxytetracycline, and doxycycline); and carbapenem antibiotics (ertapenem, doripenem, imipenem/cilastatin, and meropenem).

For example, in certain non-limiting embodiments, a microbial consortium can be administered with one or more than one antiviral agent selected from Abacavir, Acyclovir, Adefovir, Amprenavir, Atazanavir, Cidofovir, Darunavir, Delavirdine, Didanosine, Docosanol, Efavirenz, Elvitegravir, Emtricitabine, Enfuviltide, Etravirine, Famciclovir, Foscamet, Fomivirsen, Ganciclovir, Indinavir, Idoxuridine, Lamivudine, Lopinavir Maraviroc, MK-2048, Nelfinavir, Nevirapine, Penciclovir, Raltegravir, Rilpivirine, Ritonavir, Saquinavir, Stavudine, Tenofovir Trifluridine, Valaciclovir, Valganciclovir, Vidarabine, Ibacitabine, Amantadine, Oseltamivir, Rimantidine, Tipranavir, Zalcitabine, Zanamivir, and Zidovudine.

In certain embodiments, a microbial consortium can be administered with one or more than one antifungal agent selected from miconazole, ketoconazole, clotrimazole, econazole, omoconazole, bifonazole, butoconazole, fenticonazole, isoconazole, oxiconazole, sertaconazole, sulconazole, and tioconazole; triazole antifungals such as fluconazole, itraconazole, isavuconazole, ravuconazole, posaconazole, voriconazok, terconazole, and albaconazole; thiazole antifungals such as abafungin; allylamine antifungals such as terbinafine, naftifine, and butenafine; and echinocandin antifungals such as anidulafungin, caspofungin, and micafungin; polygodial; benzoic acid; ciclopirox; tolnaftate; undecylenic acid; flucytosine or 5-fluorocytosine; griseofulvin; and haloprogin.

In certain embodiments, a microbial consortium can be administered with one or more than one anti-inflammatory and/or immunosuppressive agent selected from cyclophosphamide, mycophenolate mofetil, corticosteroids, mesalazine, mesalamine, sulfasalazine, sulfasalazine derivatives, cyclosporin A, mercaptopurine, azathiopurine, prednisone, methotrexate, antihistamines, glucocorticoids, epinephrine, theophylline, cromolyn sodium, anti-leukotrienes, anticholinergics, monoclonal anti-IgE, immunomodulatory peptides, immunomodulatory small molecules, immunomodulatory cytokines, immunomodulatory antibodies, and vaccines.

In certain embodiments, a Consortia can be administered with one or more than one prebiotic selected from, but not limited to, amino acids, biotin, fructooligosaccharides, galactooligosaccharides, inulin, lactulose, mannan oligosaccharides, oligofructose-enriched inulin, oligofructose, oligodextrose, tagatose, trans-galactooligosaccharide, and xylooligosaccharides.

In certain embodiments, a Consortia described herein is administered in combination with NOV-001 (Novome). In certain embodiments, the Consortia is administered prior to the administration of NOV-001 (Novome). In certain embodiments, the Consortia is administered after to the administration of NOV-001 (Novome). In certain embodiments, the Consortia is administered concurrently with the administration of NOV-001 (Novome). In certain embodiments, the consortia administered in combination with NOV-001 (Novome) is FB-001.

In certain embodiments, a Consortia is administered in combination with SYNB8802 (Synlogic). In certain embodiments, the Consortia is administered prior to the administration of SYNB8802 (Synlogic). In certain embodiments, the Consortia is administered after to the administration of SYNB8802 (Synlogic). In certain embodiments, the Consortia is administered concurrently with the administration of SYNB8802 (Synlogic). In certain embodiments, the consortia administered in combination with SYNB8802 (Synlogic) is FB-001.

In certain embodiments, a Consortia is administered in combination with OX-1 (Oxidien). In certain embodiments, the Consortia is administered prior to the administration of OX-1 (Oxidien). In certain embodiments, the Consortia is administered after to the administration of OX-1 (Oxidien). In certain embodiments, the Consortia is administered concurrently with the administration of OX-1 (Oxidien). In certain embodiments, the consortia administered in combination with OX-1 (Oxidien) is FB-001.

In certain embodiments, a Consortia is administered in combination with Lumasiran (Alnylam). In certain embodiments, the Consortia is administered prior to the administration of Lumasiran (Alnylam). In certain embodiments, the Consortia is administered after to the administration of Lumasiran (Alnylam). In certain embodiments, the Consortia is administered concurrently with the administration of Lumasiran (Alnylam). In certain embodiments, the consortia administered in combination with Lumasiran (Alnylam) is FB-001.

In certain embodiments, a Consortia is administered in combination with Nedosiran (Dicerna). In certain embodiments, the Consortia is administered prior to the administration of Nedosiran (Dicerna). In certain embodiments, the Consortia is administered after to the administration of Nedosiran (Dicerna). In certain embodiments, the Consortia is administered concurrently with the administration of Nedosiran (Dicerna). In certain embodiments, the consortia administered in combination with Nedosiran (Dicerna) is FB-001.

In certain embodiments, a Consortia is administered in combination with BBP-711 (Cantero/Bridge Bio). In certain embodiments, the Consortia is administered prior to the administration of BBP-711 (Cantero/Bridge Bio). In certain embodiments, the Consortia is administered after to the administration of BBP-711 (Cantero/Bridge Bio). In certain embodiments, the Consortia is administered concurrently with the administration of BBP-711 (Cantero/Bridge Bio). In certain embodiments, the consortia administered in combination with BBP-711 (Cantero/Bridge Bio) is FB-001.

In certain embodiments, a Consortia is administered in combination with CNK-336 (Chinook). In certain embodiments, the Consortia is administered prior to the administration of CNK-336 (Chinook). In certain embodiments, the Consortia is administered after to the administration of CNK-336 (Chinook). In certain embodiments, the Consortia is administered concurrently with the administration of CNK-336 (Chinook). In certain embodiments, the consortia administered in combination with CNK-336 (Chinook) is FB-001.

In certain embodiments, a Consortia is administered in combination with PBGENE-PH1 (Precision Bio). In certain embodiments, the Consortia is administered prior to the administration of PBGENE-PH1 (Precision Bio). In certain embodiments, the Consortia is administered after to the administration of PBGENE-PH1 (Precision Bio). In certain embodiments, the Consortia is administered concurrently with the administration of PBGENE-PH1 (Precision Bio). In certain embodiments, the consortia administered in combination with PBGENE-PH1 (Precision Bio) is FB-001.

In certain embodiments, a Consortia is administered in combination with a low oxalate diet. In certain embodiments, a Consortia is administered in combination with a high hydration diet. In certain embodiments, a Consortia is administered in combination with calcium supplements. In certain embodiments, a Consortia is administered in combination with a low oxalate diet and with calcium supplements. In certain embodiments, the Consortia is FB-001 and FB-001 is administered in combination with a low oxalate diet, with calcium supplements, or with a low oxalate diet and calcium supplements. In certain embodiments, calcium supplements comprise a diet with sufficient calcium without additional supplementation.

In certain embodiments, a Consortia is administered in combination with 1) one of NOV-001, 2) OX-1, (Oxidien), Lumasiran (Alnylam), Nedosiran (Dicerna), BBP-711 (Cantero/Bridge Bio), CNK-336 (Chinook), and PBGENE-PH1 (Precision Bio), and 2) a low oxalate diet. In certain embodiments, a Consortia is administered in combination with 1) one of NOV-001, 2) OX-1, (Oxidien), Lumasiran (Alnylam), Nedosiran (Dicerna), BBP-711 (Cantero/Bridge Bio), CNK-336 (Chinook), and PBGENE-PH1 (Precision Bio), and 2) a high calcium diet (including but not limited to calcium supplements). In certain embodiments, a Consortia is administered in combination with 1) one of NOV-001, 2) OX-1, (Oxidien), Lumasiran (Alnylam), Nedosiran (Dicerna), BBP-711 (Cantero/Bridge Bio), CNK-336 (Chinook), and PBGENE-PH1 (Precision Bio), 2) a low oxalate diet, and 3) a high calcium diet (including but not limited to calcium supplements). In certain embodiments, FB-001 is administered in combination with 1) one of NOV-001, 2) OX-1, (Oxidien), Lumasiran (Alnylam), Nedosiran (Dicerna), BBP-711 (Cantero/Bridge Bio), CNK-336 (Chinook), and PBGENE-PH1 (Precision Bio), and 2) a low oxalate diet. In certain embodiments, FB-001 is administered in combination with 1) one of NOV-001, 2) OX-1, (Oxidien), Lumasiran (Alnylam), Nedosiran (Dicerna), BBP-711 (Cantero/Bridge Bio), CNK-336 (Chinook), and PBGENE-PH1 (Precision Bio), and 2) a high calcium diet (including but not limited to calcium supplements). In certain embodiments, FB-001 is administered in combination with 1) one of NOV-001, 2) OX-1, (Oxidien), Lumasiran (Alnylam), Nedosiran (Dicerna), BBP-711 (Cantero/Bridge Bio), CNK-336 (Chinook), and PBGENE-PH1 (Precision Bio), 2) a low oxalate diet, and 3) a high calcium diet (including but not limited to calcium supplements). In certain embodiments within this paragraph, “in combination” refers to concurrent, prior to, or after the administration of a Consortia. In certain embodiments within this paragraph, “in combination” refers to concurrent, prior to, or after the administration of FB-001.

In certain embodiments, the combination treatment of a Consortia comprises the pretreatment with antibiotics. In certain embodiments, the pretreatment of antibiotics comprises a 2, 3, 4, 5, 6, or 7 day pretreatment. In certain embodiments, the pretreatment is 4, 5, or 6 days. In certain embodiments, the pretreatment is 5 days. In certain embodiments, the pretreatment of antibiotics comprises 500 mg metronidazole. In certain embodiments, the pretreatment of antibiotics comprises 500 mg clarithromycin. In certain embodiments, the pretreatment of antibiotics comprises 500 mg metronidazole and 500 mg clarithromycin. In certain embodiments, the pretreatment of antibiotics consists of 500 mg metronidazole and 500 mg clarithromycin. In certain embodiments, the dose of antibiotics may be adjusted based on the body mass of a subject. In certain embodiments, the 500 mg metronidazole and 500 mg clarithromycin are administered every 12 hrs (Q12h). In certain embodiments, there is a 1 day gap between the last dose of antibiotics and the administration of a Consortia. In certain embodiments, there is a 2 day gap between the last dose of antibiotics and the administration of a Consortia. In certain embodiments, metronidazole and/or clarithromycin may be substituted for one or more different antibiotics with a similar or substantially similar mode of action (e.g., type of anti-bacterial). In certain embodiments, metronidazole and/or clarithromycin may be substituted for one or more different antibiotics with a similar or substantially similar mode of action (e.g., type of anti-bacterial) if a subject has a sensitivity or allergy to metronidazole and/or clarithromycin, respectively. In certain embodiments, the Consortia is FB-001. In certain embodiments, the Consortia is FB-001 and the pretreatment is 500 mg metronidazole and 500 mg clarithromycin administered as a 5 day Q12h pretreatment. In certain embodiments, the Consortia is FB-001 and the pretreatment is 500 mg metronidazole and 500 mg clarithromycin administered as a 5 day Q12h pretreatment with a 1 day gap between the administration of the last dose of the antibiotics and the first dose of FB-001. In certain embodiments, the Consortia is FB-001 and the pretreatment is 500 mg metronidazole and 500 mg clarithromycin administered as a 5 day Q12h pretreatment with no gap between the administration of the last dose of the antibiotics and the first dose of FB-001.

In certain embodiments, a bowel preparation (e.g., MiraLax) is administered in the late afternoon or early evening following the final dose of antibiotics, wherein the final dose of antibiotics is administered the morning of the same day. In certain embodiments, a bowel preparation (e.g., MiraLax) is administered in the late afternoon or early evening following the final dose of 500 mg metronidazole and 500 mg clarithromycin, wherein the final dose of 500 mg metronidazole and 500 mg clarithromycin is administered the morning of the same day. In certain embodiments, the MiraLax is administered at least 8 hrs after the last dose of 500 mg metronidazole and 500 mg clarithromycin. In certain embodiments, metronidazole and/or clarithromycin may be substituted for one or more different antibiotics with a similar or substantially similar mode of action (e.g., type of anti-bacterial). In certain embodiments, the bowel prep is MiraLax. In certain embodiments, 238 g of MiraLax is administered. In certain embodiments, the MiraLax is mixed with a flavored hydration beverage such as Gatorade, a sugar-free Gatorade, or a similar brand of alike. In certain embodiments, the MiraLax is mixed with approximately 2 L of a flavored hydration beverage. In certain embodiments, the MiraLax is mixed with approximately 1.5-2 L of a flavored hydration beverage. In certain embodiments, the MiraLax is mixed with approximately 1.9 L of a flavored hydration beverage. In certain embodiments, the diluted MiraLax is consumed by the subject at approximately 8 oz every 10-20 min. In certain embodiments, the diluted MiraLax is consumed by the subject at approximately 8 oz every 10-15 min. In certain embodiments, the diluted MiraLax is fully consumed by the subject within 90-150 min. In certain embodiments, the diluted MiraLax is fully consumed by the subject within 100-140 min. In certain embodiments, the diluted MiraLax is fully consumed by the subject within 100-130 min. In certain embodiments, the diluted MiraLax is fully consumed by the subject within 100-120 min. In certain embodiments, the diluted MiraLax is fully consumed by the subject within 120 min. In certain embodiments, the Consortia is FB-001. In certain embodiments, the MiraLax pretreatment comprises 238 g of MiraLax mixed (i.e., diluted) in approximately 1.9 L of a flavored hydration beverage (e.g., zero sugar Gatorade) that is fully consumed by the subject within approximately 120 min (e.g., 8 oz every 10-20 min) at least 8 hrs following the last dose of 500 mg metronidazole and 500 mg clarithromycin; wherein a Consortia is administered the day following the MiraLax administration. In certain embodiments, the Consortia is FB-001 and the MiraLax pretreatment comprises 238 g of MiraLax mixed (i.e., diluted) in approximately 1.9 L of a flavored hydration beverage (e.g., zero sugar Gatorade) that is fully consumed by the subject within approximately 120 min (e.g., 8 oz every 10-20 min) at least 8 hrs following the last dose of 500 mg metronidazole and 500 mg clarithromycin; wherein FB-001 is administered the day following the MiraLax administration.

### Kits

The presently disclosed subject matter provides kits for treating hyperoxaluria, enteric hyperoxaluria, primary hyperoxaluria, and secondary hyperoxaluria in a subject. In certain embodiments, the kit comprises an effective amount of presently disclosed Consortia or a pharmaceutical composition comprising thereof. In certain embodiments, the kit comprises an effective amount of FB-001 or a pharmaceutical composition comprising thereof. In certain embodiments, the kit comprises an effective amount of a functionally equivalent Consortia to FB-001 or a pharmaceutical composition comprising thereof. In certain embodiments, the kit comprises an effective amount of a functionally identical Consortia to FB-001 or a pharmaceutical composition comprising thereof. In certain embodiments, the kit comprises an effective amount of a substantially similar Consortia to FB-001 or a pharmaceutical composition comprising thereof. In certain embodiments, the kit comprises an effective amount of a similar Consortia to FB-001 or a pharmaceutical composition comprising thereof. In certain embodiments, the kit comprises a sterile container; such containers can be boxes, ampules, bottles, vials, tubes, bags, pouches, blister-packs, or other suitable container forms known in the art. Such containers can be made of plastic, glass, laminated paper, metal foil, or other materials suitable for holding medicaments. In certain non-limiting embodiments, the kit includes anaerobic containers to hold the Consortia(s) described herein. In certain non-limiting embodiments, the kit includes blister packs to hold the Consortia(s) described herein in the presence of no or limited amounts of oxygen. In certain non-limiting embodiments, the kit includes blister packs with desiccant to hold the Consortia(s) described herein in the presence of no or limited amounts of oxygen. In certain non-limiting embodiments, the kit includes bottles with desiccant to hold the Consortia(s) described herein in the presence of no or limited amounts of oxygen.

In certain embodiments, the kits include instructions for administering the Consortia as described herein. In certain embodiments, the instructions include directions for administering the loading and the maintenance dose.

In certain embodiments, the kits include storage instructions. In certain embodiments, the storage instructions are for storage at approximately −20° C. In certain embodiments, the storage instructions are for storage at less than −5° C. In certain embodiments, the storage instructions are for storage at less than approximately −15 to −20° C., −10 to −20° C., −10 to −15° C., −5 to −10° C., 0 to −5° C., below 0° C., or 0 to −20° C.

In certain embodiments, the storage instructions are for storage at less than approximately 4° C. In certain embodiments, the storage instructions are for storage at room temperature.

In certain embodiments, the kits include instructions for maintaining the Consortia in no or low oxygen conditions.

In certain embodiments, the kits include instructions for a low oxalate and/or high calcium diet.

In certain embodiments, the kits include instructions for remaining hydrated.

In certain embodiments, the kits include instructions for the subject to remain off all antibiotics during treatment with the Consortia.

In certain embodiments, the kit includes FB-001 and instructions for administering FB-001.

### EXEMPLARY EMBODIMENTS

In certain non-limiting embodiments, the present disclosure is directed to a composition comprising a microbial consortia comprising at least 1 oxalate-metabolizing microbial strain, wherein the at least one strain expresses an enzyme selected from a formyl-CoA transferase, an oxalate-formate antiporter, and an oxalyl-CoA decarboxylase.

In certain embodiments of the compositions disclosed herein, the at least 1 oxalate-metabolizing microbial strain is from the Oxalobacter genus.

In certain embodiments of the compositions disclosed herein, the composition comprises at least 3 oxalate-metabolizing microbial strains, wherein the at least 3 oxalate-metabolizing microbial strains are different strains of the same species.

In certain embodiments of the compositions disclosed herein, the composition comprises at least 3 oxalate-metabolizing microbial strains, wherein the at least 3 oxalate-metabolizing microbial strains are different strains of different species.

In certain embodiments of the compositions disclosed herein, the species is Oxalobacter formigenes (O. formigenes), and optionally wherein the number of oxalate-metabolizing microbial strains is 3 or more.

In certain embodiments of the compositions disclosed herein:

a) at least one strain is a low pH tolerance strain;

b) at least one strain is a high oxalate tolerance strain; and/or

c) at least one strain is a high growth rate strain.

In certain non-limiting embodiments, the present disclosure is directed to a composition comprising at least 2 Oxalobacter formigenes (O. formigenes) strains, wherein each of the strains comprises one or more of the following functions:

a) a low pH tolerance strain;

b) a high oxalate tolerance strain; and/or

c) a high growth rate strain.

In certain non-limiting embodiments, the present disclosure is directed to a composition comprising at least 3 Oxalobacter formigenes (O. formigenes) strains, wherein: a) at least one strain is a low pH tolerance strain; b) at least one strain is a high oxalate tolerance strain; and c) at least one strain is a high growth rate strain.

In certain embodiments of the compositions disclosed herein, the low pH tolerance strain can metabolize oxalate at a pH between about 4 and about 6.

In certain embodiments of the compositions disclosed herein, the low pH tolerance strain can metabolize oxalate at a pH of about 5.

In certain embodiments of the compositions disclosed herein, the high oxalate tolerance strain can metabolize oxalate at a concentration between about 5 mM to about 30 mM.

In certain embodiments of the compositions disclosed herein, the high oxalate tolerance strain can metabolize oxalate at a concentration of about 15 mM.

In certain embodiments of the compositions disclosed herein, each strain comprises a 16s RNA nucleotide sequence that is (a) at least about 80% identical to the nucleotide sequence set forth in SEQ ID NO: 42, SEQ ID NO: 79, or SEQ ID NO: 146, (b) at least about 90% identical to the nucleotide sequence set forth in SEQ ID NO: 42, SEQ ID NO: 79, or SEQ ID NO: 146, or (c) at least about 96% identical to the nucleotide sequence set forth in SEQ ID NO: 42, SEQ ID NO: 79, or SEQ ID NO: 146.

In certain embodiments of the compositions disclosed herein, each strain comprises a 16s RNA nucleotide sequence that is at least about 97% identical or 98.5% identical to the nucleotide sequence set forth in SEQ ID NO: 42, SEQ ID NO: 79, or SEQ ID NO: 146.

In certain embodiments of the compositions disclosed herein, each strain comprises a 16s RNA nucleotide sequence identical to the nucleotide sequence set forth in SEQ ID NO: 42, SEQ ID NO: 79, or SEQ ID NO: 146.

In certain embodiments of the compositions disclosed herein, the composition further comprises one or more microbes metabolizing formate.

In certain embodiments of the compositions disclosed herein, the composition further comprises one or more microbes catalyzing fermentation of polysaccharides.

In certain embodiments of the compositions disclosed herein, the composition further comprises one or more microbes catalyzing fermentation of amino acids.

In certain embodiments of the compositions disclosed herein, the composition further comprises microbes catalyzing the synthesis of at least one molecules selected from the group consisting of methane, acetate, sulfide, propionate, and succinate.

In certain embodiments of the compositions disclosed herein, the composition further comprises microbes catalyzing: a) deconjugation of conjugated bile acids to produce primary bile acids; b) conversion of cholic acid (CA) to 7-oxocholic acid; c) conversion of 7-oxocholic acid to 7-beta-cholic acid (7betaCA); d) conversion of chenodeoxycholic acid (CDCA) to 7-oxochenodeoxycholic acid; and/or e) conversion of 7-oxochenodeoxycholic acid to ursodeoxycholic acid (UDCA).

In certain embodiments of the compositions disclosed herein, the composition comprises: a) Consortia I or a functional equivalent thereof, b) Consortia II or a functional equivalent thereof; c) Consortia III or a functional equivalent thereof, d) Consortia IV or a functional equivalent thereof; e) Consortia V or a functional equivalent thereof, f) Consortia VI or a functional equivalent thereof, g) Consortia VII or a functional equivalent thereof, h) Consortia VIII or a functional equivalent thereof, i) Consortia IX or a functional equivalent thereof, j) Consortia X or a functional equivalent thereof; k) Consortia XI or a functional equivalent thereof; l) Consortia XII or a functional equivalent thereof, m) Consortia XIII or a functional equivalent thereof, n) Consortia XIV or a functional equivalent thereof; o) Consortia XV or a functional equivalent thereof, p) Consortia XVI or a functional equivalent thereof, q) Consortia XVII or a functional equivalent thereof, r) Consortia XVIII or a functional equivalent thereof, or s) Consortia XIX or a functional equivalent thereof.

In certain embodiments of the compositions disclosed herein, the composition further comprises a second composition comprising Clostridium citroniae, Bacteroides salyersiae, Blautia obeum, Parabacteroides merdae, Parabacteroides distasonis, Anaerostipes hadrus, Lachnospiraceae sp. FBI00033, Eubacterium eligens, Bifidobacterium dentium, Blautia wexlerae, Fusicatenibacter saccharivorans, Bacteroides nordii, Dorea formicigenerans, Dorea longicatena, Bacteroides stercorirosoris, Bifidobacterium longum, Bacteroides kribbi, Lachnospiraceae sp. FBI00071, Bacteroides thetaiotaomicron, Clostridium clostridioforme, Clostridium scindens, Roseburia hominis, Clostridium fessum, Coprococcus comes, Blautia faecis, Hungatella hathewayi, Bacteroides stercoris, Collinsella aerofaciens, Hungatella effluvii, Bifidobacterium adolescentis, Bifidobacterium catenulatum, Lactobacillus rogosae, Bacteroides faecis, Bacteroides finegoldii, Clostridiaceae sp. FBI00191, Ruminococcus faecis, Lachnoclostridium pacaense, Clostridium bolteae, Longicatena caecimuris, Eggerthella lenta, Blautia massiliensis, Bacteroides xylanisolvens, Bacteroides vulgatus, Megasphaera massiliensis, Butyricimonas faecihominis, Eisenbergiella tayi, Acidaminococcus intestini, Emergencia timonensis, Bifidobacterium pseudocatenulatum, Eubacterium hallii, Anaerofustis stercorihominis, Eubacterium ventriosum, Blautia hydrogenotrophica, Lachnospiraceae sp. FBI00290, or a functional equivalent microbial consortium.

In certain embodiments of the compositions disclosed herein, the composition further comprises FBI00001, FBI00002, FBI00010, FBI00013, FBI00029, FBI00032, FBI00033, FBI00034, FBI00043, FBI00044, FBI00048, FBI00050, FBI00051, FBI00057, FBI00059, FBI00060, FBI00070, FBI00071, FBI00076, FBI00079, FBI00087, FBI00093, FBI00102, FBI00109, FBI00117, FBI00120, FBI00125, FBI00127, FBI00128, FBI00145, FBI00162, FBI00174, FBI00184, FBI00190, FBI00191, FBI00194, FBI00198, FBI00199, FBI00200, FBI00201, FBI00205, FBI00206, FBI00211, FBI00220, FBI00221, FBI00236, FBI00245, FBI00248, FBI00251, FBI00254, FBI00267, FBI00278, FBI00288, FBI00290, or a functional equivalent thereof.

In certain embodiments of the compositions disclosed herein, each strain comprises a 16s RNA nucleotide sequence that is (a) at least about 80% identical to the nucleotide sequence set forth in SEQ ID NO: 1, SEQ ID NO: 2, SEQ ID NO: 5, SEQ ID NO: 8, SEQ ID NO: 18, SEQ ID NO: 20, SEQ ID NO: 21, SEQ ID NO: 22, SEQ ID NO: 25, SEQ ID NO: 26, SEQ ID NO: 29, SEQ ID NO: 31, SEQ ID NO: 32, SEQ ID NO: 36, SEQ ID NO: 37, SEQ ID NO: 38, SEQ ID NO: 45, SEQ ID NO: 46, SEQ ID NO: 48, SEQ ID NO: 51, SEQ ID NO: 55, SEQ ID NO: 57, SEQ ID NO: 61, SEQ ID NO: 63, SEQ ID NO: 70, SEQ ID NO: 71, SEQ ID NO: 74, SEQ ID NO: 76, SEQ ID NO: 77, SEQ ID NO: 83, SEQ ID NO: 89, SEQ ID NO: 94, SEQ ID NO: 100, SEQ ID NO: 102, SEQ ID NO: 103, SEQ ID NO: 104, SEQ ID NO: 106, SEQ ID NO: 107, SEQ ID NO: 108, SEQ ID NO: 109, SEQ ID NO: 110, SEQ ID NO: 111, SEQ ID NO: 113, SEQ ID NO: 115, SEQ ID NO: 116, SEQ ID NO: 123, SEQ ID NO: 128, SEQ ID NO: 129, SEQ ID NO: 130, SEQ ID NO: 131, SEQ ID NO: 136, SEQ ID NO: 143, SEQ ID NO: 145, or SEQ ID NO: 147, (b) at least about 90% identical to the nucleotide sequence set forth in SEQ ID NO: 1, SEQ ID NO: 2, SEQ ID NO: 5, SEQ ID NO: 8, SEQ ID NO: 18, SEQ ID NO: 20, SEQ ID NO: 21, SEQ ID NO: 22, SEQ ID NO: 25, SEQ ID NO: 26, SEQ ID NO: 29, SEQ ID NO: 31, SEQ ID NO: 32, SEQ ID NO: 36, SEQ ID NO: 37, SEQ ID NO: 38, SEQ ID NO: 45, SEQ ID NO: 46, SEQ ID NO: 48, SEQ ID NO: 51, SEQ ID NO: 55, SEQ ID NO: 57, SEQ ID NO: 61, SEQ ID NO: 63, SEQ ID NO: 70, SEQ ID NO: 71, SEQ ID NO: 74, SEQ ID NO: 76, SEQ ID NO: 77, SEQ ID NO: 83, SEQ ID NO: 89, SEQ ID NO: 94, SEQ ID NO: 100, SEQ ID NO: 102, SEQ ID NO: 103, SEQ ID NO: 104, SEQ ID NO: 106, SEQ ID NO: 107, SEQ ID NO: 108, SEQ ID NO: 109, SEQ ID NO: 110, SEQ ID NO: 111, SEQ ID NO: 113, SEQ ID NO: 115, SEQ ID NO: 116, SEQ ID NO: 123, SEQ ID NO: 128, SEQ ID NO: 129, SEQ ID NO: 130, SEQ ID NO: 131, SEQ ID NO: 136, SEQ ID NO: 143, SEQ ID NO: 145, or SEQ ID NO: 147, or (c) at least about 96% identical to the nucleotide sequence set forth in SEQ ID NO: 1, SEQ ID NO: 2, SEQ ID NO: 5, SEQ ID NO: 8, SEQ ID NO: 18, SEQ ID NO: 20, SEQ ID NO: 21, SEQ ID NO: 22, SEQ ID NO: 25, SEQ ID NO: 26, SEQ ID NO: 29, SEQ ID NO: 31, SEQ ID NO: 32, SEQ ID NO: 36, SEQ ID NO: 37, SEQ ID NO: 38, SEQ ID NO: 45, SEQ ID NO: 46, SEQ ID NO: 48, SEQ ID NO: 51, SEQ ID NO: 55, SEQ ID NO: 57, SEQ ID NO: 61, SEQ ID NO: 63, SEQ ID NO: 70, SEQ ID NO: 71, SEQ ID NO: 74, SEQ ID NO: 76, SEQ ID NO: 77, SEQ ID NO: 83, SEQ ID NO: 89, SEQ ID NO: 94, SEQ ID NO: 100, SEQ ID NO: 102, SEQ ID NO: 103, SEQ ID NO: 104, SEQ ID NO: 106, SEQ ID NO: 107, SEQ ID NO: 108, SEQ ID NO: 109, SEQ ID NO: 110, SEQ ID NO: 111, SEQ ID NO: 113, SEQ ID NO: 115, SEQ ID NO: 116, SEQ ID NO: 123, SEQ ID NO: 128, SEQ ID NO: 129, SEQ ID NO: 130, SEQ ID NO: 131, SEQ ID NO: 136, SEQ ID NO: 143, SEQ ID NO: 145, or SEQ ID NO: 147.

In certain embodiments of the compositions disclosed herein, each strain comprises a 16s RNA nucleotide sequence that is at least about 97% identical or 98.5% identical to the nucleotide sequence set forth in SEQ ID NO: 1, SEQ ID NO: 2, SEQ ID NO: 5, SEQ ID NO: 8, SEQ ID NO: 18, SEQ ID NO: 20, SEQ ID NO: 21, SEQ ID NO: 22, SEQ ID NO: 25, SEQ ID NO: 26, SEQ ID NO: 29, SEQ ID NO: 31, SEQ ID NO: 32, SEQ ID NO: 36, SEQ ID NO: 37, SEQ ID NO: 38, SEQ ID NO: 45, SEQ ID NO: 46, SEQ ID NO: 48, SEQ ID NO: 51, SEQ ID NO: 55, SEQ ID NO: 57, SEQ ID NO: 61, SEQ ID NO: 63, SEQ ID NO: 70, SEQ ID NO: 71, SEQ ID NO: 74, SEQ ID NO: 76, SEQ ID NO: 77, SEQ ID NO: 83, SEQ ID NO: 89, SEQ ID NO: 94, SEQ ID NO: 100, SEQ ID NO: 102, SEQ ID NO: 103, SEQ ID NO: 104, SEQ ID NO: 106, SEQ ID NO: 107, SEQ ID NO: 108, SEQ ID NO: 109, SEQ ID NO: 110, SEQ ID NO: 111, SEQ ID NO: 113, SEQ ID NO: 115, SEQ ID NO: 116, SEQ ID NO: 123, SEQ ID NO: 128, SEQ ID NO: 129, SEQ ID NO: 130, SEQ ID NO: 131, SEQ ID NO: 136, SEQ ID NO: 143, SEQ ID NO: 145, or SEQ ID NO: 147.

In certain embodiments of the compositions disclosed herein, each strain comprises a 16s RNA nucleotide sequence identical to the nucleotide sequence set forth in SEQ ID NO: SEQ ID NO: 1, SEQ ID NO: 2, SEQ ID NO: 5, SEQ ID NO: 8, SEQ ID NO: 18, SEQ ID NO: 20, SEQ ID NO: 21, SEQ ID NO: 22, SEQ ID NO: 25, SEQ ID NO: 26, SEQ ID NO: 29, SEQ ID NO: 31, SEQ ID NO: 32, SEQ ID NO: 36, SEQ ID NO: 37, SEQ ID NO: 38, SEQ ID NO: 45, SEQ ID NO: 46, SEQ ID NO: 48, SEQ ID NO: 51, SEQ ID NO: 55, SEQ ID NO: 57, SEQ ID NO: 61, SEQ ID NO: 63, SEQ ID NO: 70, SEQ ID NO: 71, SEQ ID NO: 74, SEQ ID NO: 76, SEQ ID NO: 77, SEQ ID NO: 83, SEQ ID NO: 89, SEQ ID NO: 94, SEQ ID NO: 100, SEQ ID NO: 102, SEQ ID NO: 103, SEQ ID NO: 104, SEQ ID NO: 106, SEQ ID NO: 107, SEQ ID NO: 108, SEQ ID NO: 109, SEQ ID NO: 110, SEQ ID NO: 111, SEQ ID NO: 113, SEQ ID NO: 115, SEQ ID NO: 116, SEQ ID NO: 123, SEQ ID NO: 128, SEQ ID NO: 129, SEQ ID NO: 130, SEQ ID NO: 131, SEQ ID NO: 136, SEQ ID NO: 143, SEQ ID NO: 145, or SEQ ID NO: 147.

In certain embodiments of the compositions disclosed herein, the composition further comprises a third composition comprising Acutalibacter timonensis, Alistipes onderdonkii, Bacteroides uniformis, Eubacterium rectale, Alistipes timonensis, Bacteroides kribbi, Coprococcus eutactus, Bilophila wadsworthia, Bacteroides caccae, Alistipes shahii, Parasutterella excrementihominis, Paraprevotella clara, Sutterella wadsworthensis, Sutterella massiliensis, Porphyromonas asaccharolytica, Ruminococcus bromii, Monoglobus pectinilyticus, Ruminococcaceae sp. FBI00097, Gordonibacter pamelaeae, Bacteroides uniformis, Gordonibacter pamelaeae, Bacteroides fragilis, Phascolarctobacterium faecium, Monoglobus pectinilyticus, Clostridium aldenense, Ruthenibacterium lactatiformans, Bacteroides ovatus, Bifidobacterium bifidum, Anaerotruncus massiliensis, Clostridium aldenense, Sutterella wadsworthensis, Catabacter hongkongensis, Alistipes senegalensis, Ruminococcaceae sp. FBI00233, Alistipes shahii, Dielma fastidiosa, Eubacterium siraeum, Faecalibacterium prausnitzii, Turicibacter sanguinis, Eubacterium rectale, Bacteroides caccae, Methanobrevibacter smithii, Barnesiella intestinihominis, Alistipes onderdonkii, Methanobrevibacter smithii, or a functional equivalent thereof.

In certain embodiments of the compositions disclosed herein, the composition further comprises FBI00004, FBI00012, FBI00015, FBI00018, FBI00019, FBI00021, FBI00038, FBI00040, FBI00046, FBI00061, FBI00066, FBI00075, FBI00077, FBI00080, FBI00081, FBI00085, FBI00092, FBI00097, FBI00099, FBI00112, FBI00132, FBI00137, FBI00140, FBI00149, FBI00151, FBI00176, FBI00189, FBI00197, FBI00208, FBI00212, FBI00224, FBI00226, FBI00229, FBI00233, FBI00235, FBI00237, FBI00243, FBI00244, FBI00258, FBI00260, FBI00263, FBI00270, FBI00273, FBI00277, FBI00292, or a functional equivalent thereof.

In certain embodiments of the compositions disclosed herein, each strain comprises a 16s RNA nucleotide sequence that is (a) at least about 80% identical to the nucleotide sequence set forth in SEQ ID NO: 3, SEQ ID NO: 7, SEQ ID NO: 9, SEQ ID NO: 11, SEQ ID NO: 12, SEQ ID NO: 14, SEQ ID NO: 23, SEQ ID NO: 24, SEQ ID NO: 27, SEQ ID NO: 39, SEQ ID NO: 41, SEQ ID NO: 47, SEQ ID NO: 49, SEQ ID NO: 50, SEQ ID NO: 51, SEQ ID NO: 54, SEQ ID NO: 56, SEQ ID NO: 59, SEQ ID NO: 60, SEQ ID NO: 66, SEQ ID NO: 78, SEQ ID NO: 81, SEQ ID NO: 82, SEQ ID NO: 85, SEQ ID NO: 86, SEQ ID NO: 96, SEQ ID NO: 101, SEQ ID NO: 105, SEQ ID NO: 112, SEQ ID NO: 114, SEQ ID NO: 117, SEQ ID NO: 118, SEQ ID NO: 119, SEQ ID NO: 121, SEQ ID NO: 122, SEQ ID NO: 124, SEQ ID NO: 126, SEQ ID NO: 127, SEQ ID NO: 133, SEQ ID NO: 134, SEQ ID NO: 135, SEQ ID NO: 138, SEQ ID NO: 140, SEQ ID NO: 142, or SEQ ID NO: 148, (b) at least about 90% identical to the nucleotide sequence set forth in SEQ ID NO: 3, SEQ ID NO: 7, SEQ ID NO: 9, SEQ ID NO: 11, SEQ ID NO: 12, SEQ ID NO: 14, SEQ ID NO: 23, SEQ ID NO: 24, SEQ ID NO: 27, SEQ ID NO: 39, SEQ ID NO: 41, SEQ ID NO: 47, SEQ ID NO: 49, SEQ ID NO: 50, SEQ ID NO: 51, SEQ ID NO: 54, SEQ ID NO: 56, SEQ ID NO: 59, SEQ ID NO: 60, SEQ ID NO: 66, SEQ ID NO: 78, SEQ ID NO: 81, SEQ ID NO: 82, SEQ ID NO: 85, SEQ ID NO: 86, SEQ ID NO: 96, SEQ ID NO: 101, SEQ ID NO: 105, SEQ ID NO: 112, SEQ ID NO: 114, SEQ ID NO: 117, SEQ ID NO: 118, SEQ ID NO: 119, SEQ ID NO: 121, SEQ ID NO: 122, SEQ ID NO: 124, SEQ ID NO: 126, SEQ ID NO: 127, SEQ ID NO: 133, SEQ ID NO: 134, SEQ ID NO: 135, SEQ ID NO: 138, SEQ ID NO: 140, SEQ ID NO: 142, or SEQ ID NO: 148, or (c) at least about 96% identical to the nucleotide sequence set forth in SEQ ID NO: 3, SEQ ID NO: 7, SEQ ID NO: 9, SEQ ID NO: 11, SEQ ID NO: 12, SEQ ID NO: 14, SEQ ID NO: 23, SEQ ID NO: 24, SEQ ID NO: 27, SEQ ID NO: 39, SEQ ID NO: 41, SEQ ID NO: 47, SEQ ID NO: 49, SEQ ID NO: 50, SEQ ID NO: 51, SEQ ID NO: 54, SEQ ID NO: 56, SEQ ID NO: 59, SEQ ID NO: 60, SEQ ID NO: 66, SEQ ID NO: 78, SEQ ID NO: 81, SEQ ID NO: 82, SEQ ID NO: 85, SEQ ID NO: 86, SEQ ID NO: 96, SEQ ID NO: 101, SEQ ID NO: 105, SEQ ID NO: 112, SEQ ID NO: 114, SEQ ID NO: 117, SEQ ID NO: 118, SEQ ID NO: 119, SEQ ID NO: 121, SEQ ID NO: 122, SEQ ID NO: 124, SEQ ID NO: 126, SEQ ID NO: 127, SEQ ID NO: 133, SEQ ID NO: 134, SEQ ID NO: 135, SEQ ID NO: 138, SEQ ID NO: 140, SEQ ID NO: 142, or SEQ ID NO: 148.

In certain embodiments of the compositions disclosed herein, each strain comprises a 16s RNA nucleotide sequence that is at least about 97% identical or 98.5% identical to the nucleotide sequence set forth in SEQ ID NO: 3, SEQ ID NO: 7, SEQ ID NO: 9, SEQ ID NO: 11, SEQ ID NO: 12, SEQ ID NO: 14, SEQ ID NO: 23, SEQ ID NO: 24, SEQ ID NO: 27, SEQ ID NO: 39, SEQ ID NO: 41, SEQ ID NO: 47, SEQ ID NO: 49, SEQ ID NO: 50, SEQ ID NO: 51, SEQ ID NO: 54, SEQ ID NO: 56, SEQ ID NO: 59, SEQ ID NO: 60, SEQ ID NO: 66, SEQ ID NO: 78, SEQ ID NO: 81, SEQ ID NO: 82, SEQ ID NO: 85, SEQ ID NO: 86, SEQ ID NO: 96, SEQ ID NO: 101, SEQ ID NO: 105, SEQ ID NO: 112, SEQ ID NO: 114, SEQ ID NO: 117, SEQ ID NO: 118, SEQ ID NO: 119, SEQ ID NO: 121, SEQ ID NO: 122, SEQ ID NO: 124, SEQ ID NO: 126, SEQ ID NO: 127, SEQ ID NO: 133, SEQ ID NO: 134, SEQ ID NO: 135, SEQ ID NO: 138, SEQ ID NO: 140, SEQ ID NO: 142, or SEQ ID NO: 148.

In certain embodiments of the compositions disclosed herein, each strain comprises a 16s RNA nucleotide sequence identical to the nucleotide sequence set forth in SEQ ID NO: SEQ ID NO: 3, SEQ ID NO: 7, SEQ ID NO: 9, SEQ ID NO: 11, SEQ ID NO: 12, SEQ ID NO: 14, SEQ ID NO: 23, SEQ ID NO: 24, SEQ ID NO: 27, SEQ ID NO: 39, SEQ ID NO: 41, SEQ ID NO: 47, SEQ ID NO: 49, SEQ ID NO: 50, SEQ ID NO: 51, SEQ ID NO: 54, SEQ ID NO: 56, SEQ ID NO: 59, SEQ ID NO: 60, SEQ ID NO: 66, SEQ ID NO: 78, SEQ ID NO: 81, SEQ ID NO: 82, SEQ ID NO: 85, SEQ ID NO: 86, SEQ ID NO: 96, SEQ ID NO: 101, SEQ ID NO: 105, SEQ ID NO: 112, SEQ ID NO: 114, SEQ ID NO: 117, SEQ ID NO: 118, SEQ ID NO: 119, SEQ ID NO: 121, SEQ ID NO: 122, SEQ ID NO: 124, SEQ ID NO: 126, SEQ ID NO: 127, SEQ ID NO: 133, SEQ ID NO: 134, SEQ ID NO: 135, SEQ ID NO: 138, SEQ ID NO: 140, SEQ ID NO: 142, or SEQ ID NO: 148.

In certain embodiments of the compositions disclosed herein, the composition further comprises a fourth composition comprising Bifidobacterium adolescentis, Bifidobacterium longum, Bifidobacterium pseudocatenulatum, Bacteroides thetaiotaomicron, Coprococcus comes, Fusicatenibacter saccharivorans, Eggerthella lenta, Eubacterium eligens, Bacteroides xylanisolvens, Lactobacillus rogosae, Clostridium citroniae, Collinsella aerofaciens, Blautia obeum, Eggerthella lenta, Blautia wexlerae, Lachnoclostridium pacaense, Bacteroides vulgatus, Parabacteroides merdae, Dorea formicigenerans, Ruminococcus faecis, Roseburia hominis, Anaerostipes hadrus, Bifidobacterium adolescentis, Bifidobacterium pseudocatenulatum, Clostridium bolteae, Eisenbergiella tayi, Dorea longicatena, Eggerthella lenta, Bacteroides stercoris, Hungatella hathewayi, Bacteroides xylanisolvens, or a functional equivalent thereof.

In certain embodiments of the compositions disclosed herein, the composition further comprises FBI00009, FBI00011, FBI00016, FBI00020, FBI00025, FBI00027, FBI00030, FBI00047, FBI00052, FBI00053, FBI00056, FBI00062, FBI00078, FBI00096, FBI00104, FBI00110, FBI00111, FBI00113, FBI00115, FBI00116, FBI00123, FBI00124, FBI00126, FBI00135, FBI00147, FBI00159, FBI00167, FBI00170, FBI00232, FBI00255, FBI00271, or a functional equivalent thereof.

In certain embodiments of the compositions disclosed herein, each strain comprises a 16s RNA nucleotide sequence that is (a) at least about 80% identical to the nucleotide sequence set forth in SEQ ID NO: 4, SEQ ID NO: 6, SEQ ID NO: 10, SEQ ID NO: 13, SEQ ID NO: 16, SEQ ID NO: 17, SEQ ID NO: 19, SEQ ID NO: 28, SEQ ID NO: 33, SEQ ID NO: 34, SEQ ID NO: 35, SEQ ID NO: 40, SEQ ID NO: 50, SEQ ID NO: 58, SEQ ID NO: 62, SEQ ID NO: 64, SEQ ID NO: 65, SEQ ID NO: 67, SEQ ID NO: 68, SEQ ID NO: 69, SEQ ID NO: 72, SEQ ID NO: 73, SEQ ID NO: 75, SEQ ID NO: 80, SEQ ID NO: 84, SEQ ID NO: 88, SEQ ID NO: 91, SEQ ID NO: 92, SEQ ID NO: 120, SEQ ID NO: 132, or SEQ ID NO: 139, (b) at least about 90% identical to the nucleotide sequence set forth in SEQ ID NO: 4, SEQ ID NO: 6, SEQ ID NO: 10, SEQ ID NO: 13, SEQ ID NO: 16, SEQ ID NO: 17, SEQ ID NO: 19, SEQ ID NO: 28, SEQ ID NO: 33, SEQ ID NO: 34, SEQ ID NO: 35, SEQ ID NO: 40, SEQ ID NO: 50, SEQ ID NO: 58, SEQ ID NO: 62, SEQ ID NO: 64, SEQ ID NO: 65, SEQ ID NO: 67, SEQ ID NO: 68, SEQ ID NO: 69, SEQ ID NO: 72, SEQ ID NO: 73, SEQ ID NO: 75, SEQ ID NO: 80, SEQ ID NO: 84, SEQ ID NO: 88, SEQ ID NO: 91, SEQ ID NO: 92, SEQ ID NO: 120, SEQ ID NO: 132, or SEQ ID NO: 139, or (c) at least about 96% identical to the nucleotide sequence set forth in SEQ ID NO: 4, SEQ ID NO: 6, SEQ ID NO: 10, SEQ ID NO: 13, SEQ ID NO: 16, SEQ ID NO: 17, SEQ ID NO: 19, SEQ ID NO: 28, SEQ ID NO: 33, SEQ ID NO: 34, SEQ ID NO: 35, SEQ ID NO: 40, SEQ ID NO: 50, SEQ ID NO: 58, SEQ ID NO: 62, SEQ ID NO: 64, SEQ ID NO: 65, SEQ ID NO: 67, SEQ ID NO: 68, SEQ ID NO: 69, SEQ ID NO: 72, SEQ ID NO: 73, SEQ ID NO: 75, SEQ ID NO: 80, SEQ ID NO: 84, SEQ ID NO: 88, SEQ ID NO: 91, SEQ ID NO: 92, SEQ ID NO: 120, SEQ ID NO: 132, or SEQ ID NO: 139.

In certain embodiments of the compositions disclosed herein, each strain comprises a 16s RNA nucleotide sequence that is at least about 97% identical or 98.5% identical to the nucleotide sequence set forth in SEQ ID NO: 4, SEQ ID NO: 6, SEQ ID NO: 10, SEQ ID NO: 13, SEQ ID NO: 16, SEQ ID NO: 17, SEQ ID NO: 19, SEQ ID NO: 28, SEQ ID NO: 33, SEQ ID NO: 34, SEQ ID NO: 35, SEQ ID NO: 40, SEQ ID NO: 50, SEQ ID NO: 58, SEQ ID NO: 62, SEQ ID NO: 64, SEQ ID NO: 65, SEQ ID NO: 67, SEQ ID NO: 68, SEQ ID NO: 69, SEQ ID NO: 72, SEQ ID NO: 73, SEQ ID NO: 75, SEQ ID NO: 80, SEQ ID NO: 84, SEQ ID NO: 88, SEQ ID NO: 91, SEQ ID NO: 92, SEQ ID NO: 120, SEQ ID NO: 132, or SEQ ID NO: 139.

In certain embodiments of the compositions disclosed herein, each strain comprises a 16s RNA nucleotide sequence identical to the nucleotide sequence set forth in SEQ ID NO: SEQ ID NO: 4, SEQ ID NO: 6, SEQ ID NO: 10, SEQ ID NO: 13, SEQ ID NO: 16, SEQ ID NO: 17, SEQ ID NO: 19, SEQ ID NO: 28, SEQ ID NO: 33, SEQ ID NO: 34, SEQ ID NO: 35, SEQ ID NO: 40, SEQ ID NO: 50, SEQ ID NO: 58, SEQ ID NO: 62, SEQ ID NO: 64, SEQ ID NO: 65, SEQ ID NO: 67, SEQ ID NO: 68, SEQ ID NO: 69, SEQ ID NO: 72, SEQ ID NO: 73, SEQ ID NO: 75, SEQ ID NO: 80, SEQ ID NO: 84, SEQ ID NO: 88, SEQ ID NO: 91, SEQ ID NO: 92, SEQ ID NO: 120, SEQ ID NO: 132, or SEQ ID NO: 139.

In certain embodiments of the compositions disclosed herein, the composition further comprises a fifth composition comprising Alistipes putredinis, Dialister succinatiphilus, Akkermansia muciniphila, Ruminococcus bromii, Dialister invisus, Bacteroides massiliensis, Bilophila wadsworthia, Holdemanella biformis, Parasutterella excrementihominis, Alistipes sp. FBI00180, Bacteroides coprocola, Alistipes sp. FBI00238, Alistipes putredinis, Eubacterium xylanophilum, Senegalimassilia anaerobia, or a functional equivalent thereof.

In certain embodiments of the compositions disclosed herein, the composition further comprises FBI00022, FBI00049, FBI00068, FBI00069, FBI00152, FBI00165, FBI00171, FBI00175, FBI00177, FBI00180, FBI00182, FBI00238, FBI00269, FBI00274, FBI00281, or a functional equivalent thereof.

In certain embodiments of the compositions disclosed herein, each strain comprises a 16s RNA nucleotide sequence that is (a) at least about 80% identical to the nucleotide sequence set forth in SEQ ID NO: 15, SEQ ID NO: 30, SEQ ID NO: 43, SEQ ID NO: 44, SEQ ID NO: 87, SEQ ID NO: 90, SEQ ID NO: 93, SEQ ID NO: 95, SEQ ID NO: 97, SEQ ID NO: 98, SEQ ID NO: 99, SEQ ID NO: 125, SEQ ID NO: 137, SEQ ID NO: 141, or SEQ ID NO: 144, (b) at least about 90% identical to the nucleotide sequence set forth in SEQ ID NO: 15, SEQ ID NO: 30, SEQ ID NO: 43, SEQ ID NO: 44, SEQ ID NO: 87, SEQ ID NO: 90, SEQ ID NO: 93, SEQ ID NO: 95, SEQ ID NO: 97, SEQ ID NO: 98, SEQ ID NO: 99, SEQ ID NO: 125, SEQ ID NO: 137, SEQ ID NO: 141, or SEQ ID NO: 144, or (c) at least about 96% identical to the nucleotide sequence set forth in SEQ ID NO: 15, SEQ ID NO: 30, SEQ ID NO: 43, SEQ ID NO: 44, SEQ ID NO: 87, SEQ ID NO: 90, SEQ ID NO: 93, SEQ ID NO: 95, SEQ ID NO: 97, SEQ ID NO: 98, SEQ ID NO: 99, SEQ ID NO: 125, SEQ ID NO: 137, SEQ ID NO: 141, or SEQ ID NO: 144.

In certain embodiments of the compositions disclosed herein, each strain comprises a 16s RNA nucleotide sequence that is at least about 97% identical or 98.5% identical to the nucleotide sequence set forth in SEQ ID NO: 15, SEQ ID NO: 30, SEQ ID NO: 43, SEQ ID NO: 44, SEQ ID NO: 87, SEQ ID NO: 90, SEQ ID NO: 93, SEQ ID NO: 95, SEQ ID NO: 97, SEQ ID NO: 98, SEQ ID NO: 99, SEQ ID NO: 125, SEQ ID NO: 137, SEQ ID NO: 141, or SEQ ID NO: 144.

In certain embodiments of the compositions disclosed herein, each strain comprises a 16s RNA nucleotide sequence identical to the nucleotide sequence set forth in SEQ ID NO: SEQ ID NO: 15, SEQ ID NO: 30, SEQ ID NO: 43, SEQ ID NO: 44, SEQ ID NO: 87, SEQ ID NO: 90, SEQ ID NO: 93, SEQ ID NO: 95, SEQ ID NO: 97, SEQ ID NO: 98, SEQ ID NO: 99, SEQ ID NO: 125, SEQ ID NO: 137, SEQ ID NO: 141, or SEQ ID NO: 144 In certain non-limiting embodiments, the present disclosure is directed to a microbial consortium comprising microbial strains set forth in Table 1, Table 2, Table 3, Table 4, Table 5, Table 6, Table 7, Table 8, Table 9, Table 10, Table 11, Table 12, Table 13, Table 14, Table 15, Table 16, Table 17, Table 18, Table 19, or a functional equivalent thereof.

In certain non-limiting embodiments, the present disclosure is directed to a microbial consortium comprising microbial strains set forth in Table 22 or a functional equivalent thereof.

In certain embodiments of the microbial consortia disclosed herein, each strain comprises a 16s RNA nucleotide sequence that is (a) at least about 80% identical to the nucleotide sequence set forth in SEQ ID NOs: 1-148, (b) at least about 90% identical to the nucleotide sequence set forth in SEQ ID NOs: 1-148, or (c) at least about 96% identical to the nucleotide sequence set forth in SEQ ID NOs: 1-148.

In certain embodiments of the microbial consortia disclosed herein, each strain comprises a 16s RNA nucleotide sequence that is at least about 97% identical or 98.5% to the nucleotide sequence set forth in SEQ ID NOs: 1-148.

In certain embodiments of the microbial consortia disclosed herein, each strain comprises a 16s RNA nucleotide sequence that is identical to the nucleotide sequence set forth in SEQ ID NOs: 1-148.

In certain non-limiting embodiments, the present disclosure is directed to a composition comprising a microbial consortium disclosed herein.

In certain embodiments of the compositions disclosed herein, the composition is a pharmaceutical composition.

In certain embodiments of the compositions disclosed herein, the composition comprises from about 5×10¹⁰ to about 5×10¹¹ viable cells.

In certain embodiments of the compositions disclosed herein, the composition comprises from about 5×10⁹ to about 5×10¹⁰ viable cells.

In certain embodiments of the compositions disclosed herein, the composition comprises from about 5×10¹¹ to about 5×10¹² viable cells.

In certain embodiments of the compositions disclosed herein, the composition comprises up to about 5×10¹² viable cells.

In certain embodiments of the compositions disclosed herein, the composition comprises from about 10% to about 50% of oxalate-metabolizing microbial strains.

In certain embodiments of the compositions disclosed herein, the composition comprises from about 10% to about 50% of O. formigenes strains on a viable cell count basis.

In certain embodiments of the compositions disclosed herein, the composition comprises about 20% of O. formigenes strains on a viable cell count basis.

In certain embodiments of the compositions disclosed herein, the composition comprises about 30% of O. formigenes strains on a viable cell count basis.

In certain embodiments of the compositions disclosed herein, the composition comprises about 40% of O. formigenes strains on a viable cell count basis.

In certain non-limiting embodiments, the present disclosure is directed to a method of manufacturing the compositions or the microbial consortia disclosed herein. In certain embodiments of the methods of manufacturing disclosed herein, the method comprises obtaining and blending:

a) a first composition comprising Clostridium citroniae, Bacteroides salyersiae, Blautia obeum, Parabacteroides merdae, Parabacteroides distasonis, Anaerostipes hadrus, Lachnospiraceae sp. FBI00033, Eubacterium eligens, Bifidobacterium dentium, Blautia wexlerae, Fusicatenibacter saccharivorans, Bacteroides nordii, Dorea formicigenerans, Dorea longicatena, Bacteroides stercorirosoris, Bifidobacterium longum, Bacteroides kribbi, Lachnospiraceae sp. FBI00071, Bacteroides thetaiotaomicron, Clostridium clostridioforme, Clostridium scindens, Roseburia hominis, Clostridium fessum, Coprococcus comes, Blautia faecis, Hungatella hathewayi, Bacteroides stercoris, Collinsella aerofaciens, Hungatella effluvii, Bifidobacterium adolescentis, Bifidobacterium catenulatum, Lactobacillus rogosae, Bacteroides faecis, Bacteroides finegoldii, Clostridiaceae sp. FBI00191, Ruminococcus faecis, Lachnoclostridium pacaense, Clostridium bolteae, Longicatena caecimuris, Eggerthella lenta, Blautia massiliensis, Bacteroides xylanisolvens, Bacteroides vulgatus, Megasphaera massiliensis, Butyricimonas faecihominis, Eisenbergiella tayi, Acidaminococcus intestini, Emergencia timonensis, Bifidobacterium pseudocatenulatum, Eubacterium hallii, Anaerofustis stercorihominis, Eubacterium ventriosum, Blautia hydrogenotrophica, and Lachnospiraceae sp. FBI00290, or a functional equivalent thereof;

b) a second composition comprising Acutalibacter timonensis, Alistipes onderdonkii, Bacteroides uniformis, Eubacterium rectale, Alistipes timonensis, Bacteroides kribbi, Coprococcus eutactus, Bilophila wadsworthia, Bacteroides caccae, Alistipes shahii, Parasutterella excrementihominis, Paraprevotella clara, Sutterella wadsworthensis, Sutterella massiliensis, Porphyromonas asaccharolytica, Ruminococcus bromii, Monoglobus pectinilyticus, Ruminococcaceae sp. FBI00097, Gordonibacter pamelaeae, Bacteroides uniformis, Gordonibacter pamelaeae, Bacteroides fragilis, Phascolarctobacterium faecium, Monoglobus pectinilyticus, Clostridium aldenense, Ruthenibacterium lactatiformans, Bacteroides ovatus, Bifidobacterium bifidum, Anaerotruncus massiliensis, Clostridium aldenense, Sutterella wadsworthensis, Catabacter hongkongensis, Alistipes senegalensis, Ruminococcaceae sp. FBI00233, Alistipes shahii, Dielma fastidiosa, Eubacterium siraeum, Faecalibacterium prausnitzii, Turicibacter sanguinis, Eubacterium rectale, Bacteroides caccae, Methanobrevibacter smithii, Barnesiella intestinihominis, Alistipes onderdonkii, and Methanobrevibacter smithii, or a functional equivalent thereof;

c) a third composition comprising Bifidobacterium adolescentis, Bifidobacterium longum, Bifidobacterium pseudocatenulatum, Bacteroides thetaiotaomicron, Coprococcus comes, Fusicatenibacter saccharivorans, Eggerthella lenta, Eubacterium eligens, Bacteroides xylanisolvens, Lactobacillus rogosae, Clostridium citroniae, Collinsella aerofaciens, Blautia obeum, Eggerthella lenta, Blautia wexlerae, Lachnoclostridium pacaense, Bacteroides vulgatus, Parabacteroides merdae, Dorea formicigenerans, Ruminococcus faecis, Roseburia hominis, Anaerostipes hadrus, Bifidobacterium adolescentis, Bifidobacterium pseudocatenulatum, Clostridium bolteae, Eisenbergiella tayi, Dorea longicatena, Eggerthella lenta, Bacteroides stercoris, Hungatella hathewayi, and Bacteroides xylanisolvens, or a functional equivalent thereof;

d) a fourth composition comprising Alistipes putredinis, Dialister succinatiphilus, Akkermansia muciniphila, Ruminococcus bromii, Dialister invisus, Bacteroides massiliensis, Bilophila wadsworthia, Holdemanella biformis, Parasutterella excrementihominis, Alistipes sp. FBI00180, Bacteroides coprocola, Alistipes sp. FBI00238, Alistipes putredinis, Eubacterium xylanophilum, and Senegalimassilia anaerobia, or a functional equivalent thereof;

e) a fifth composition comprising a first O. formigenes strain;

f) a sixth composition comprising a second O. formigenes strain; and/or

g) a seventh composition comprising a third O. formigenes strain.

In certain embodiments of the methods of manufacturing disclosed herein, the method comprises obtaining and blending:

a) a first composition comprising FBI00001, FBI00002, FBI00010, FBI00013, FBI00029, FBI00032, FBI00033, FBI00034, FBI00043, FBI00044, FBI00048, FBI00050, FBI00051, FBI00057, FBI00059, FBI00060, FBI00070, FBI00071, FBI00076, FBI00079, FBI00087, FBI00093, FBI00102, FBI00109, FBI00117, FBI00120, FBI00125, FBI00127, FBI00128, FBI00145, FBI00162, FBI00174, FBI00184, FBI00190, FBI00191, FBI00194, FBI00198, FBI00199, FBI00200, FBI00201, FBI00205, FBI00206, FBI00211, FBI00220, FBI00221, FBI00236, FBI00245, FBI00248, FBI00251, FBI00254, FBI00267, FBI00278, FBI00288, and FBI00290, or a functional equivalent thereof;

b) a second composition comprising FBI00004, FBI00012, FBI00015, FBI00018, FBI00019, FBI00021, FBI00038, FBI00040, FBI00046, FBI00061, FBI00066, FBI00075, FBI00077, FBI00080, FBI00081, FBI00085, FBI00092, FBI00097, FBI00099, FBI00112, FBI00132, FBI00137, FBI00140, FBI00149, FBI00151, FBI00176, FBI00189, FBI00197, FBI00208, FBI00212, FBI00224, FBI00226, FBI00229, FBI00233, FBI00235, FBI00237, FBI00243, FBI00244, FBI00258, FBI00260, FBI00263, FBI00270, FBI00273, FBI00277, and FBI00292, or a functional equivalent thereof;

c) a third composition comprising FBI00009, FBI00011, FBI00016, FBI00020, FBI00025, FBI00027, FBI00030, FBI00047, FBI00052, FBI00053, FBI00056, FBI00062, FBI00078, FBI00096, FBI00104, FBI00110, FBI00111, FBI00113, FBI00115, FBI00116, FBI00123, FBI00124, FBI00126, FBI00135, FBI00147, FBI00159, FBI00167, FBI00170, FBI00232, FBI00255, and FBI00271, or a functional equivalent thereof;

d) a fourth composition comprising FBI00022, FBI00049, FBI00068, FBI00069, FBI00152, FBI00165, FBI00171, FBI00175, FBI00177, FBI00180, FBI00182, FBI00238, FBI00269, FBI00274, and FBI00281, or a functional equivalent thereof;

e) a fifth composition comprising FBI00067 or a functional equivalent thereof;

f) a sixth composition comprising FBI00133 or a functional equivalent thereof, and/or

g) a seventh composition comprising FBI00289 or a functional equivalent thereof.

In certain embodiments of the methods of manufacturing disclosed herein, each strain comprises a 16s RNA nucleotide sequence that is (a) at least about 80% identical to the nucleotide sequence set forth in SEQ ID NOs: 1-148, (b) at least about 90% identical to the nucleotide sequence set forth in SEQ ID NOs: 1-148, or (c) at least about 96% identical to the nucleotide sequence set forth in SEQ ID NOs: 1-148.

In certain embodiments of the methods of manufacturing disclosed herein, each strain comprises a 16s RNA nucleotide sequence that is at least about 97% identical or 98.5% identical to the nucleotide sequence set forth in SEQ ID NOs: 1-148.

In certain embodiments of the methods of manufacturing disclosed herein, each strain comprises a 16s RNA nucleotide sequence identical to the nucleotide sequence set forth in SEQ ID NO: 1-148.

In certain embodiments of the methods of manufacturing disclosed herein, the fourth composition is obtained by growing microbes in presence of threonine.

In certain embodiments of the methods of manufacturing disclosed herein, each composition comprises a lyoprotectant.

In certain embodiments of the methods of manufacturing disclosed herein, each composition comprises maltodextrin, inulin, or a combination thereof.

In certain embodiments of the methods of manufacturing disclosed herein, the maldextrin is at a concentration of about 8%.

In certain embodiments of the methods of manufacturing disclosed herein, the inulin is at a concentration of about 0.5%.

In certain embodiments of the methods of manufacturing disclosed herein, each composition is separately lyophilized.

In certain embodiments of the methods of manufacturing disclosed herein, the functional equivalent is based on the characteristics set forth in Table 24.

In certain embodiments of the methods of manufacturing disclosed herein, the functional equivalent is based on the characteristics set forth in Table 34.

In certain embodiments of the methods of manufacturing disclosed herein, the functional equivalent is based on the characteristics set forth in Table 35.

In certain embodiments of the methods of manufacturing disclosed herein, the functional equivalent is based on the characteristics set forth in Table 36.

In certain embodiments of the methods of manufacturing disclosed herein, the functional equivalent is based on the characteristics set forth in Tables 34-36.

In certain embodiments of the methods of manufacturing disclosed herein, the method comprises obtaining and blending microbes comprising a gene regulating oxalate degradation, oxalate resistance, formate metabolism, metabolism of macronutrients, production of microbial metabolites, cross-feeding activity, and/or mucin degradation.

In certain embodiments of the methods of manufacturing disclosed herein, the method comprises obtaining and blending microbes that are known to protect against diseases and/or that are prevalent in healthy human gut.

In certain embodiments of the methods of manufacturing disclosed herein, the method comprises obtaining and blending microbes that utilize carbon sources set forth in Table 35.

In certain embodiments of the methods of manufacturing disclosed herein, each strain can optionally utilize a subset of the carbon sources set forth in Table 35.

In certain embodiments of the methods of manufacturing disclosed herein, each composition is prepared using inoculation density adjustment.

In certain embodiments of the methods of manufacturing disclosed herein, each composition is cultured or has been cultured in presence of gas overlay.

In certain embodiments of the methods of manufacturing disclosed herein, each composition is cultured or has been cultured in absence of gas sparging.

In certain non-limiting embodiments, the present disclosure is directed to a composition prepared by the methods of manufacturing disclosed herein.

In certain non-limiting embodiments, the present disclosure is directed to a method of treating hyperoxaluria in a subject in need thereof comprising administering an effective amount of the compositions or the microbial consortia disclosed herein.

In certain non-limiting embodiments, the present disclosure is directed to a method of reducing the risk of developing hyperoxaluria in a subject in need thereof comprising administering an effective amount of the compositions or the microbial consortia disclosed herein.

In certain non-limiting embodiments, the present disclosure is directed to a method of reducing urinary oxalate in a subject in need thereof comprising administering an effective amount of the compositions or the microbial consortia disclosed herein.

In certain embodiments of the methods disclosed herein, the hyperoxaluria is a primary hyperoxaluria, a secondary hyperoxaluria, or an enteric hyperoxaluria.

In certain embodiments of the methods disclosed herein, the secondary hyperoxaluria is associated with bowel resection surgery.

In certain embodiments of the methods disclosed herein, the hyperoxaluria is enteric hyperoxaluria.

In certain embodiments of the methods disclosed herein, the method further comprises administering at least one antibacterial agent, antiviral agent, antifungal agent, anti-inflammatory agent, immunosuppressive agent, prebiotic, or a combination thereof.

In certain embodiments of the methods disclosed herein, the method further comprises administering NOV-001, SYNB8802, OX-1, Lumasiran, Nedosiran, BBP-711, CNK-336, PBGENE-PH1, or a combination thereof.

In certain embodiments of the methods disclosed herein, the method further comprises administering a low oxalate diet, a high hydration diet, calcium supplements, or a combination thereof.

In certain embodiments of the methods disclosed herein, the composition or the microbial consortium is administered orally.

In certain non-limiting embodiments, the present disclosure is directed to a method of treating hyperoxaluria in a subject in need thereof comprising administering a first dose of the compositions or microbial consortia disclosed herein.

In certain non-limiting embodiments, the present disclosure is directed to a method of reducing the risk of developing hyperoxaluria in a subject in need thereof comprising administering a first dose of the compositions or microbial consortia disclosed herein.

In certain non-limiting embodiments, the present disclosure is directed to a method of reducing urinary oxalate in a subject in need thereof comprising administering a first dose of the compositions or microbial consortia disclosed herein.

In certain embodiments of the methods disclosed herein, the hyperoxaluria is a primary hyperoxaluria, a secondary hyperoxaluria, or an enteric hyperoxaluria.

In certain embodiments of the methods disclosed herein, the secondary hyperoxaluria is associated with bowel resection surgery.

In certain embodiments of the methods disclosed herein, the hyperoxaluria is enteric hyperoxaluria.

In certain embodiments of the methods disclosed herein, the method further comprises administering an antibiotic treatment.

In certain embodiments of the methods disclosed herein, the antibiotic treatment is administered for about 2 days, about 3 days, about 4 days, about 5 days, about 6 days, or about 7 days.

In certain embodiments of the methods disclosed herein, the antibiotic is metronidazole, clarithromycin, or a combination thereof.

In certain embodiments of the methods disclosed herein, the antibiotic treatment is completed 1 day before administering the first dose.

In certain embodiments of the methods disclosed herein, the antibiotic treatment is completed 2 days before administering the first dose.

In certain embodiments of the methods disclosed herein, the method further comprises administering a bowel preparation treatment.

In certain embodiments of the methods disclosed herein, the bowel preparation treatment is administered to the subject after the antibiotic treatment.

In certain embodiments of the methods disclosed herein, the bowel preparation treatment is administered before the first dose.

In certain embodiments of the methods disclosed herein, the first dose comprises an effective amount of the composition or the microbial consortium.

In certain embodiments of the methods disclosed herein, the first dose comprises about 10¹² viable cells.

In certain embodiments of the methods disclosed herein, the first dose is administered for about 1 day.

In certain embodiments of the methods disclosed herein, the first dose is administered for about 2 days.

In certain embodiments of the methods disclosed herein, the method further comprises administering a second dose of the compositions or microbial consortia disclosed herein.

In certain embodiments of the methods disclosed herein, the second dose comprises an effective amount of the composition or the microbial consortium.

In certain embodiments of the methods disclosed herein, the second dose comprises about 10¹¹ viable cells.

In certain embodiments of the methods disclosed herein, the second dose is administered up to about 8 days.

In certain embodiments of the methods disclosed herein, the second dose is administered up to about 10 days.

In certain embodiments of the methods disclosed herein, the first dose is administered orally.

In certain embodiments of the methods disclosed herein, the second dose is administered orally.

In certain non-limiting embodiments, the present disclosure is directed to a kit comprising the compositions or the microbial consortia disclosed herein.

In certain embodiments of the kits disclosed herein, the kit comprises a container comprising a desiccant.

In certain embodiments of the kits disclosed herein, the container comprises anaerobic conditions.

In certain embodiments of the kits disclosed herein, the container is a blister.

In certain embodiments of the kits disclosed herein, the kit further comprises written instructions for administering the composition or microbial consortium.

In certain non-limiting embodiments, the present disclosure is directed to a method of culturing a microbial strain from the Akkermansia genus comprising contacting the strain with N-Acetylgalactosamine (GalNAc).

In certain embodiments of the methods of culturing disclosed herein, the strain is Akkermansia muciniphilia.

In certain non-limiting embodiments, the present disclosure is directed to a microbial consortium comprising the functional properties set forth in Table 23.

In certain non-limiting embodiments, the present disclosure is directed to a microbial consortium comprising the functional properties set forth in Table 24.

In certain non-limiting embodiments, the present disclosure is directed to a microbial consortium comprising the functional properties set forth in Table 34.

In certain non-limiting embodiments, the present disclosure is directed to a microbial consortium comprising the functional properties set forth in Table 35.

In certain non-limiting embodiments, the present disclosure is directed to a microbial consortium comprising the functional properties set forth in Table 36.

In certain non-limiting embodiments, the present disclosure is directed to a microbial consortia comprising FB-001 or a functional equivalent thereof.

In certain non-limiting embodiments, the present disclosure is directed to any method or composition described herein.

### Examples

### Example 1: Design of Consortia

While microbial consortia of two or more microbial strains have been made before, limitations existed that prevent manufacturing and clinical efficacy. Specifically, manufacturing limitations have prevented the design and generation of large consortia that are able to engraft in the gastrointestinal tract and build a functional microbiota system.

Isolation of donor-derived microbial strains. Microbial strains were isolated and identified using the methods described in PCT/US2021/021790.

Generation of Consortia. Using the microbial strains identified using the isolation and identification methods described in PCT/US2021/021790, over 30 large consortia were made and examined for their functional ability to metabolize oxalate, absence of phages, acceptable endotoxin levels, and their ability to be manufactured in multi-strain drug substances. The reason for the large number of experimental large consortia was because it was unknown what combination of microbial strains would be optimal given the considerations above. More so, the optimal combination of microbial strains could not be predicted with algorithms and required wet laboratory work to determine efficacy and manufacturability.

Nineteen exemplary consortia are provided in Tables 1-19.

Drug products comprising each of the consortia above were tested for the ability to metabolize oxalate using in vitro and/or in vivo assays.

In exemplary experiments, in vitro studies were performed on germ-free mice. determine whether diet and existing gastrointestinal microbiota had an effect on the efficacy of Consortia in reducing oxalate in vivo. Germ-free mice were divided into three groups: 1) diet was a refined, sugary diet, 2) diet was a complex, grain-based diet, and 3) diet was a complex, grain-based diet and the mice were colonized with human FMT. The mice from groups 1-3 were then given one of Consortia I-VIII. The refined, sugary diet (also referred to as the Ox36 diet) consisted of 316.22 g/kg sucrose, 280 g/kg corn starch, 200 g/kg casein, 50 g/kg corn oil, 35 g/kg inulin, 35 g/kg pectin, 25 g/kg cellulose, 16.23 g/kg sodium chloride, 13.37 g/kg mineral mix (Ca—P deficient), 11.4 g/kg potassium phosphate monobasic, 10 g/kg vitamin mix (Teklad), 3.72 g/kg sodium oxalate, 3 g/kg DL-methionine, 1.05 g/kg calcium chloride, and 0.01 g/kg ethoxyquin (antioxidant). As formulated, the Ox36 diet contained 0.372% sodium oxalate, 1.88% NaCl, 2.5% cellulose, 3.5% inulin and 3.5% pectin and the nutritional breakdown of the diet was 58.3% carbohydrates, 17.7% protein, and 5.2% fat (by weight). The complex, grain-based diet consisted of 22.7% protein by weigh, 40.3% carbohydrate by weigh, 5% fat by weigh and was made using the PMI Laboratory Autoclavable Rodent Diet (Envigo Cat No 5010) with the addition of sodium oxalate and sodium chloride (final product consisting of 970.82 g/Kg PMI Laboratory Autoclavable Rodent Diet, 21.5 g/Kg sodium oxalate, and 7.68 g/Kg sodium chloride).

In these experiments, the germ-free C57Bl/6 mice are fed either the refined, sugary diet or the complex, grain-based diet to induce hyperoxaluria. After one week, one of Consortias I-VIII were introduced via oral gavage to the mice. Mice were sampled thereafter to determine microbiome composition and urinary oxalate levels. Specifically, on day −7, the mice began the diets, on day 0 the mice were gavaged, on day 7 fecal samples were taken and food consumption was measured, and on day 14 the mice were taken down to collect urine and feces and serum samples, cecal images, and kidney/liver inspection and/or images were taken when possible. The negative control for these experiments were a gavage with PBS instead of a Consortia.

Oxalate and creatinine were measured by LC-MS/MS from urine samples acquired on day 14.

Representative data from mice fed the complex, grain-based diet that were gavaged with Consortias is provided in Tables 20 and 21.

TABLE 20
Oxalate urine Concentrations, μM

| Treatment | R1 | R2 | R3 | R4 | R5 | Ave | SD | CV, % |
|---|---|---|---|---|---|---|---|---|
| PBS Control | 9,157 | 3,962 | 6,452 | 6,999 | 6,778 | 6,669 | 1,850 | 27.73 |
| Consortia I | 1,313 | 1,858 | 3,874 | 3,517 | | 2,640 | 1,247 | 47.23 |
| Consortia II | 2,118 | 3,270 | 4,237 | 3,422 | | 3,261 | 873 | 26.76 |
| Consortia III | 1,468 | 1,783 | 2,153 | 2,353 | | 1,939 | 393 | 20.27 |
| Consortia IV | 972 | 1,596 | 1,249 | 713 | | 1,132 | 379 | 33.46 |
| Consortia V | 1,173 | 750 | 846 | 801 | | 893 | 191 | 21.42 |

TABLE 21
Creatinine Urine Concentrations, μM

| Treatment | R1 | R2 | R3 | R4 | R5 | Ave | SD | CV, % |
|---|---|---|---|---|---|---|---|---|
| PBS Control | 2,563 | 3,346 | 3,977 | 4,129 | 2,906 | 3,384 | 673 | 19.88 |
| Consortia I | 1,417 | 1,413 | 1,820 | 1,975 | | 1,656 | 286 | 17.25 |
| Consortia II | 1,702 | 1,822 | 2,218 | 1,476 | | 1,805 | 311 | 17.22 |
| Consortia III | 1,449 | 1,363 | 1,661 | 1,723 | | 1,549 | 171 | 11.03 |
| Consortia IV | 1,219 | 1,304 | 1,190 | 1,245 | | 1,239 | 49 | 3.92 |
| Consortia V | 923 | 897 | 1,264 | 1,250 | | 1,084 | 201 | 18.51 |

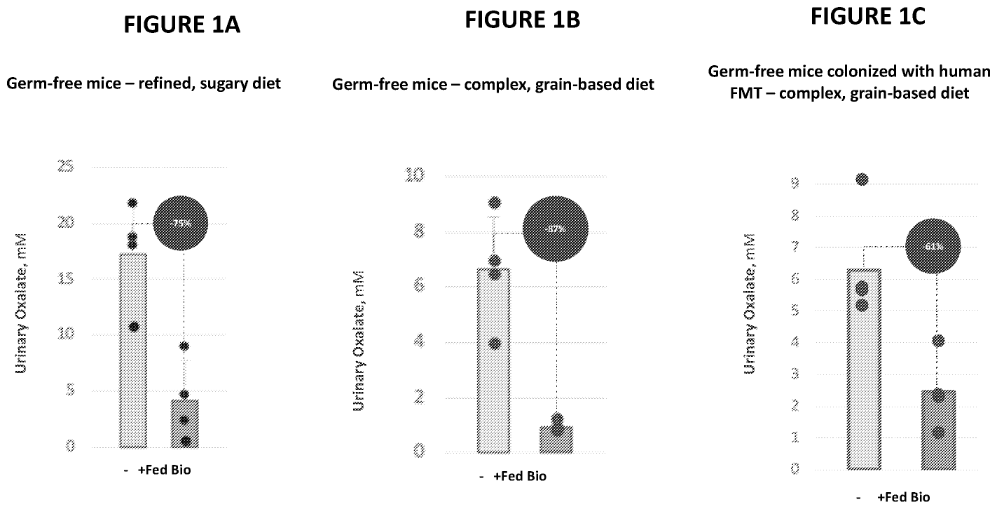

Furthermore, it was surprising to see that the ability of the Consortia to reduce urinary oxalate was independent of diet. Representative data from mice fed either the complex grain based diet or the refined sugary diet that were gavaged with Consortia VI (FIG. 1A) or VIII (FIG. 1B) show the ability of the Consortia to reduce urinary oxalate is independent of diet.

An additional question that was unknown was whether existing microbiota in the gastrointestinal tract would affect the efficacy of the Consortias. Accordingly, the experiments described above were repeated in germ-free mice that were colonized with human FMT prior to the initiation of the oxalate diets (either the refined, sugary diet or the complex, grain-based diet). As shown in FIG. 1C, the pre-existing microbiota did not affect the efficacy of the Consortia (Consortia VII, in this example). The ability of the Consortia to have an active effect on the reduction of urinary oxalate levels regardless of the existing microbiota was unexpected because literature suggested that it was necessary to eliminate existing microbiota using antibiotics in order for microbiome products to engraft and function in a gastrointestinal tract.

While Table 20 shows that Consortia V was most effective at oxalate metabolism and degradation (i.e., Consortia V had the lowest concentration of urinary oxalate), additional investigation and modification of the Consortia was needed to design a product for the treatment of disease, specifically a disease that causes or is caused by decrease ability or inability to effectively metabolize and degrade oxalate in the gastrointestinal tract. Accordingly, modifications of Consortia V were made to determine which microbiota provided functional benefits, including but not limited consortia growth, oxalate metabolism and degradation, consortia engraftment, and consortia survival, and which microbiota were either not needed or provided a detriment to the patient receiving the consortia as treatment of the disease or a detriment to the function of the consortia as a whole (including but not limited consortia growth, oxalate metabolism and degradation, consortia engraftment, and consortia survival). Examples of such designed and investigated consortia are Consortia IX-XVI.

Of Consortia IX-XVI that were designed and tested, Consortia IX was selected as the lead for clinical development. Key changes made as variations of the consortia were made to modify for the treatment of disease, specifically a disease that causes or is caused by decrease ability or inability to effectively metabolize and degrade oxalate in the gastrointestinal tract, include removing the Citrobacter freundii strain because through experimentation it was determined to be facultative anaerobes (see e.g., strain removal between Consortia XIII and XV and between Consortia XXIV and XIII and XII), replacement of one Bacteroides kribbi species with a different Bacteroides kribbi species cluster (see e.g., strain replacements between Consortia XV and XVI), replacement of one Blautia faecis species with a different Blautia faecis species (see e.g., strain replacements between Consortia XV and XVI), strains that were determined to be duplicative strains based on Whole Genome Sequencing cluster (see e.g., strain removals between Consortia XVII and XVI), replacement of one Bifidobacterium adolescentis with an alternate Bifidobacterium adolescentis to improve growth in culture (see e.g., strain replacement between Consortia X and XII), replacement of one Bifidobacterium pseudocatenulatum with an alternate Bifidobacterium pseudocatenulatum to improve growth in culture (see e.g., strain replacement between Consortia X and XII), replacement of one Bacteroides xylanisolvens with an alternate Bacteroides xylanisolvens to improve growth in culture (see e.g., strain replacement between Consortia X and XII), replacement of one Clostridium citroniae with an alternate Clostridium citroniae to improve growth in culture (see e.g., strain replacement between Consortia X and XII), replacement of one Blautia faecis with an alternate Blautia faecis in order to identify a Blautia strain that was able to grow sufficiently to produce a master cell bank (see e.g., strain replacement between Consortia X and XII), removal of Holdemanella biformis to eliminate phage risk because while a phage was not detected in co-culture it was detected using bioinformatic methods (see e.g., strain replacement between Consortia X and XII), and removal of Faecalibacterium prausnitzii to eliminate phage risk because while a phage was not detected in co-culture it was detected using bioinformatic methods (see e.g., strain replacement between Consortia X and XII).

### Example 2: Oxalobacter formigenes Microbiota

Oxalobacter formigenes (O. formigenes) is a key active microbiota for the degradation and metabolism of oxalate and it is included in the Consortia I-XIX. However, as shown in Tables 1-19 above, certain Consortia have O. formigenes listed three times in each of the Consortia. The reason for this is because there are multiple strains of O. formigenes and it was determined through experimentation that the different strains identified had different physiologies that directly affected engraftment and function in the gastrointestinal tract. The three O. formigenes strains that were selected for Consortia I-XIX comprise 1) one strain with a low pH tolerance, 2) one strain with a high oxalate tolerance, and 3) one strain that has a high growth rate.

While any set of O. formigenes strains that meet the criteria of 1-3 above can be used in a consortium designed to increase oxalate metabolism and degradation, the strains used in Consortia I-XIX comprise the 16S RNA sequences of SEQ ID NO:42, SEQ ID NO: 79, and SEQ ID NO: 146.

### Example 3: Drug Product Design and Manufacture

As shown in Example 1, the Consortia described herein were designed to be a complex community of anaerobic microbiota that can engraft and function in a gastrointestinal tract. However, prior methods known to one of skill in the art were not capable of manufacturing such large consortia. Accordingly, new methods of manufacture were needed in order to grow the microbiota in discrete groups (i.e., drug substances) to then form a final drug product.

Conventionally, Live Biotherapeutic Products (LBPs) are manufactured one strain at a time (i.e., single strain manufacturing). Single strain manufacture necessitates fermentation scale-up of each single strain followed by lyophilization to make individual drug substances (each a “DS”). Thereafter the multiple DSs of individual lyophilized stains are then blended into a mixture and filled into capsules or other suitable packaging/filling to make a final drug product (a “DP”). While this works for small consortia, it is not feasible to grow 100+ strains separately, make 100+ DSs, and then blend 100+ DSs into a stable DP. In addition to stability limitations, current technology would require 1 or more year(s) to manufacture a single DP. Accordingly, conventional manufacturing using current technology was not an option for a DP comprising 100+ strains, and preferably 145+ strains as provided in Consortia IX.

As the Consortia were designed and modified as described in Examples 1 and 2, manufacturing methods were developed that were capable of manufacturing the 145+ strain consortia that comprise over 90 species, and 4 or more or the 6 taxonomic phyla found in the human gastrointestinal tract microbiome. More so, methods were developed to modify for Consortia IX that comprises approximately 99 species across the taxonomic phyla of Bacteroidetes, Firmicutes, Actinobacteria, Proteobacteria, and Archaea. The methods developed and described herein are mixed co-culture methods that are capable of stably growing greater than 50 strains in one co-culture to generate DSs with greater than 50 strains.

Strains were selected for co-culture by based on growth rates and the manufacturing was initially designed to add strains to the co-culture at different times throughout the manufacturing process in order to achieve optimal growth of each strain. This approach was termed “time of addition” manufacturing. The rationale behind this initial approach was to ensure the strains reanimate in the gastrointestinal tract to increase efficacy of engraftment (i.e., allow for engraftment before the strains are excreted. Optimal reanimation and engraftment of the lyophilized strains require preserving the strains in an “active state” (i.e., active growth state). However, this “time of addition” manufacturing approach was not successful because growth rates of the strains in the Consortia described herein are highly variable which makes it difficult to achieve exponential growth simultaneously for diverse strains in coculture. Accordingly, it was determined that additional experimentation was needed in order to understand each strain's unique growth kinetics to enable binning of strains based on growth rate and further modification of time of addition to the bioreactor. Growth kinetic assays were performed using HTP anaerobic growth kinetic assays on each individual strain in each of Consortia IX-XVI at 8 different inoculation densities.

While experimentation to understand each strain's unique growth kinetics proved helpful with the time of addition manufacturing, ultimately the highly variable nature of growing strains from a lyophilized powder to an active consortia in a bioreactor proved undesirable for the time of addition methods.

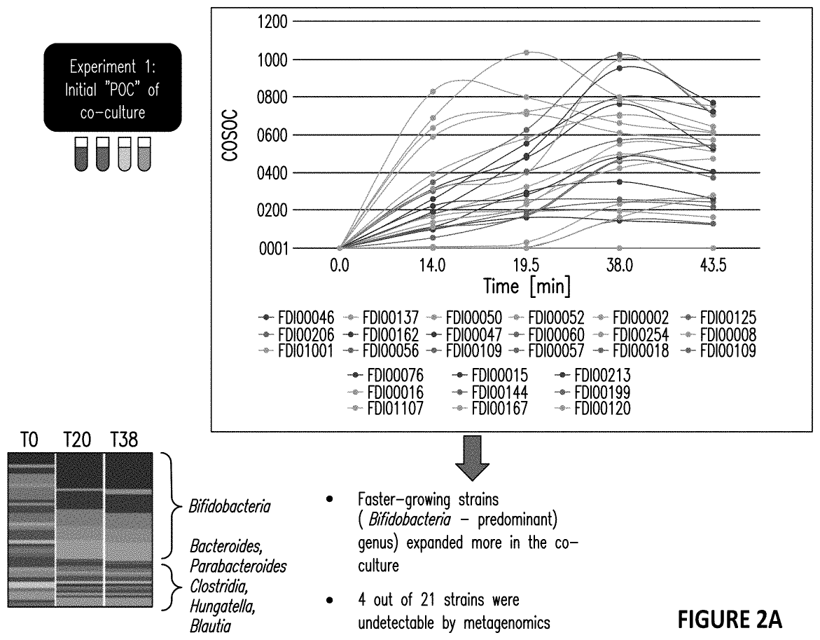

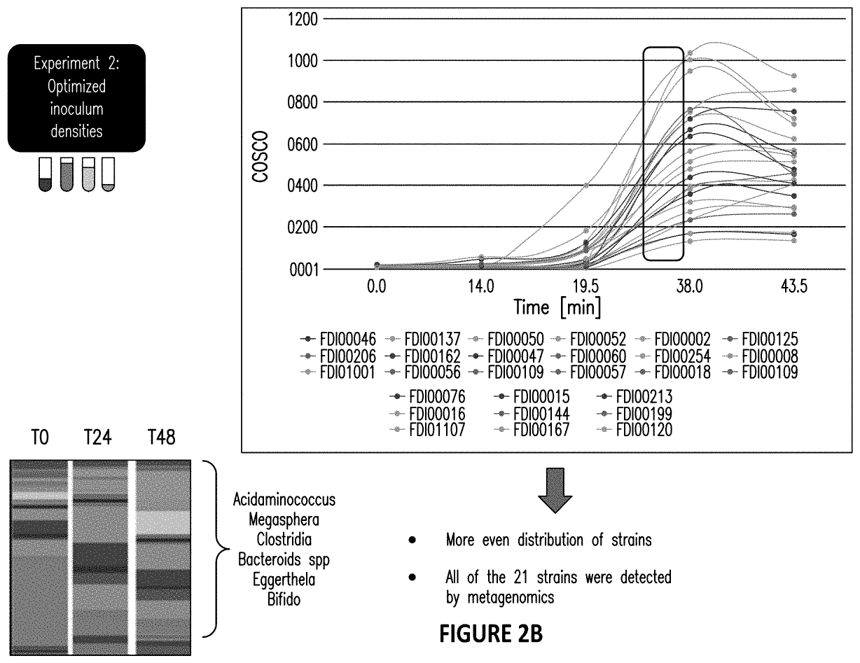

Accordingly, a second approach for coculture was developed. Instead of applying different time of additions, the second approach used inoculation density adjustment for each strain to synchronize growth and control of strain distribution at the time of harvest from the co-culture (“inoculation density” manufacturing). Using the unique growth kinetics determined for each strain in the Consortia, specifically Consortia IX-XVI, optimal growth zones were determined for each strain. In doing so, it was determined that coculture was effective and possible if each strain was added to culture at an initial time point based on inoculum density (i.e., number of cells per strain added to the co-culture) such that higher inoculum densities of certain strains resulted in shorter growth lag time for such strains. Based on this, higher inoculum density of slow growing strains and lower inoculum density of fast growing strains resulted in a synchronized harvest time. As shown by means of example in FIGS. 2A and 2B, modifying inoculation densities of individual strains allowed control over the strain distribution and improved strain recovery in cocultures (i.e., even distribution of strains as well as higher number of strain recovery are achieved by adjusting inoculum densities). FIG. 2A shows an example of a co-culture of 21 fast growing strains where only 4 of the 21 strains were undetectable by metagenomics in the final product. However, it is important to note that even if a strain is not detected in the final product, the strain may still provide a community advantage to allow for more efficient and robust growth of other strains that are detectable in the final product. FIG. 2B shows a further modified experiment of that show in FIG. 2A where the time of harvest and strain detection was modified. As shown the different timing of growth and culture led to a better distribution of strains and detection of all 21 strains.

Further modification of the coculture process was needed to improve fermentation. For example, additional modification was performed to control for pH and to achieve conditions of growth based on the bioreactor container (i.e., the type of container and the size of the container).

Using the methods developed and described herein, Consortia IX-XVI were each manufactured using only 7 DSs. One exemplary 7 DS Drug Product comprises: 3 O. formigenes monocultures (see the 3 phenotypes of the 3 O. formigenes cultures described in Example 2), the strains of DS1 (e.g., listed in Table 22), the strains of DS2 (e.g., listed in Table 22), the strains of DS3 (e.g., listed in Table 22), the strains of DS4 (e.g., listed in Table 22).

In order to identify each DS without sequencing the entire genome of all strains and in order to ensure proper growth throughout the coculture process, identifier strains were developed. For DS1, the identifier strains were Bacteroides thetaiotaomicron, Bifidobacterium pseudocatenulatum, and Megasphaera massiliensis. For DS2, the identifier strains were Bacteroides ovatus, Faecalibacterium prausnitzii, and Phascolarctobacterium faecium. For DS3, the identifier strains were Blautia wexlerae, Anaerostipes hadrus, and Clostridium bolteae. For DS4, the identifier strains were Holdemanella biformis, Parasutterella excrementihominis, and Dialister invisus.

As described herein, the number of strains detected at the conclusion of the co-culture may be less than the number of strains added at the beginning of the culture. This may be a result of limited detection methods. Furthermore, while not all strains may be detected at the conclusion of the coculture process, the inclusion of the undetected strains may still be vital for the survival and propagation of other strains that are detected.

In one experiment, DS1 consisted of 54 initial strains and 50 strains were detected at the end of the coculture process; DS2 consisted of 47 initial strains and 39 strains were detected at the end of the coculture process; DS3 consisted of 33 initial strains and 30 strains were detected at the end of the coculture process; and DS4 consisted of 14 initial strains and 11 strains were detected at the end of the coculture process. Accordingly, in this experiment 148 strains were detectable at the beginning of the coculture and 130 strains were detected at the completion of the culture.

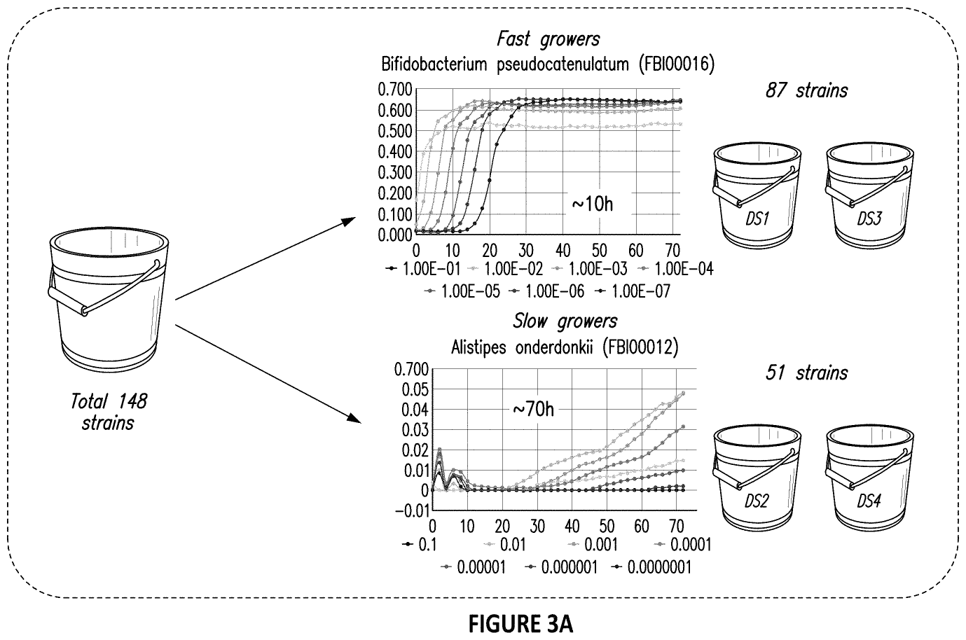

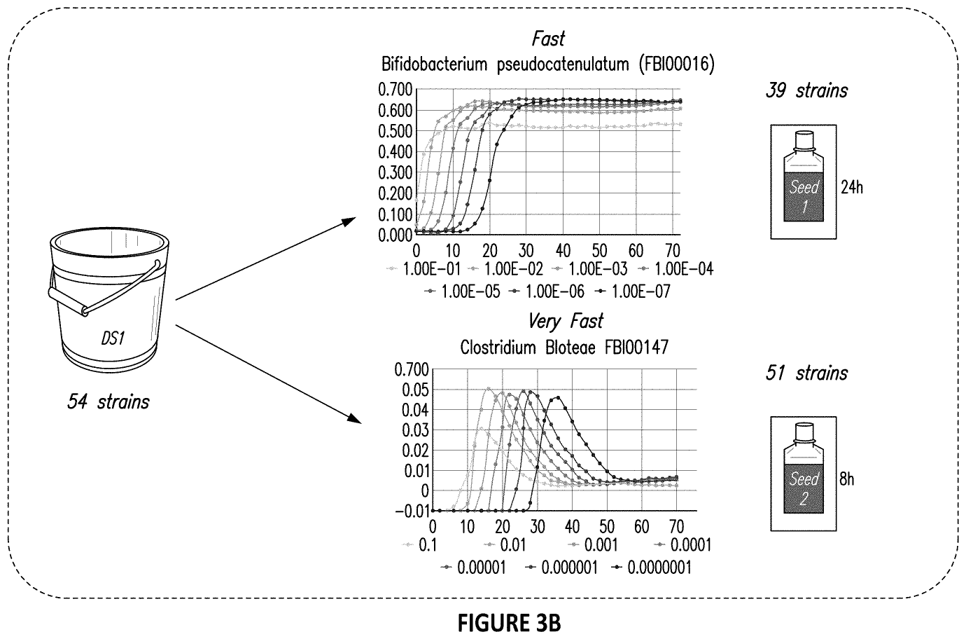

This achievement of 130/148 strains was achieved through development of a fermentation process that allowed for optimal growth of diverse strains in coculture. Variables that were investigated include growth kinetics of each strain, nutritional requirements for each strain, competition for nutritional sources in each DS, selection of the optimal starting inoculum concentration to achieve strain growth and distribution in each DS. For example growth curves were performed and used to define DS buckets as well as starting inoculum composition. This is shown in FIGS. 3A and 3B. FIG. 3A shows the design of strain segregation into 4 DS buckets based on slow and fast growing strains. FIG. 3B shows the starting inoculum seed design for fast and very fast growing strains. Using 5 iterations of the strain segregation and inoculum seed design methods, the DS1, for example, was able to increase its yield rate from approximately 35/54 strains detected at the conclusion of the coculture process to 50/54 strains detected at the conclusion of the coculture process.

Additional experimentation was required to successfully manufacture the DSs at large scale. For example, experimentation was performed on sterilization procedures and raw materials used in the media, gas solubility in the bioreactor (i.e., fermenter), shear stress caused by the impeller and gas sparging in the bioreactor, and mass transfer and mixing times. Each of these factors are necessary in order develop a process that could successfully produce a complex consortia such as any of the Consortia described herein. For example, through experimentation, it was determined that nitrogen sparging lead to higher sheering and impacted gas solubility. Accordingly, experiments were performed to adjust the speed of the sparger, location of the sparger, and replacement of sparging to gas overlay. The data showed that gas overlay was the only approach that provided successful coculture of DSs. For example, data from different sparging conditions only allowed for the detection of up to 36 out of 54 strains from DS1 while gas overlay allowed for detection of an additional 11 species at the conclusion of the coculture (i.e., 47/54 strains).

The next step in the manufacturing process that had to be developed was a method of storing the final product in a way that preserved the stability and activity of the strains. Freezing and lyophilization methods were investigated to determine what would preserve the activity and viability of the strains for each DS.

In order to determine if lyophilization would be better than freezing to preserve the activity and viability of the strains in each DS, lyophilization processes had to be developed because none were known in the art for the complexity of the DSs and Consortia provided herein. Key variables that were investigated in order to develop lyophilization process for each DS included but were not limited to: formulation of the broth or alternative microbiota suspension media, methods to prevent oxygen contamination during the lyophilization process, excipient:broth ratio, parameters for freezing the microbiota suspension prior to the lyophilization, cycle parameters for the lyophilization, sterilization requirements, methods for reviving the microbiota following lyophilized storage, buffers for reviving the microbiota, and storage of the lyophilized DS.

By means of example, high throughput, foil covered plates were used as one of the test options for storage of the lyophilized DS. This was presumed to work because the foil cover should prevent oxygen exposure. However, it was determined that foil covered plates in fact did not prevent oxygen contamination because there was no way to partially stopper the plate. Another storage method that was investigated was glass and plastic tray vials with multiplexed stoppers. The theoretical advantage of this approach was hypothesized to be the ability to do high throughput screening without the need to individually stopper each vial because the multiplexed stoppers can be pushed into the vials in a single step. However, this method proved ineffective because oxygen contamination occurred with the removal of the multiplexed stoppers. After exploring additional options for methods of preserving the lyophilized product, it was determined that individual glass vials with individual stoppers allowed for long term storage without oxygen contamination.

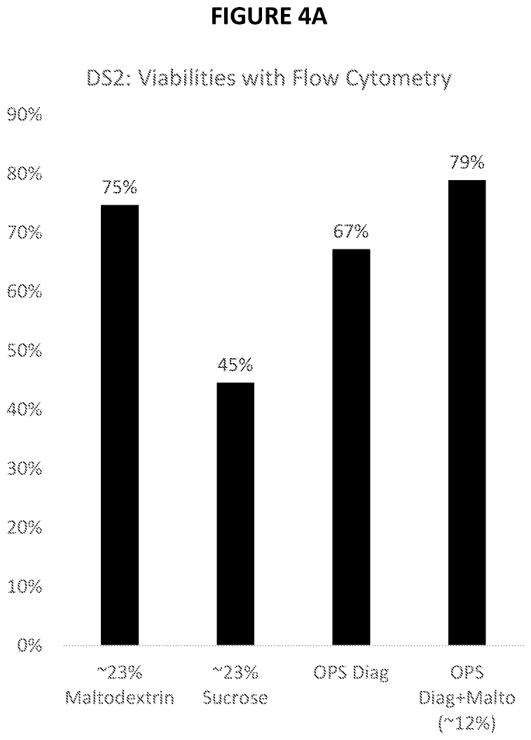

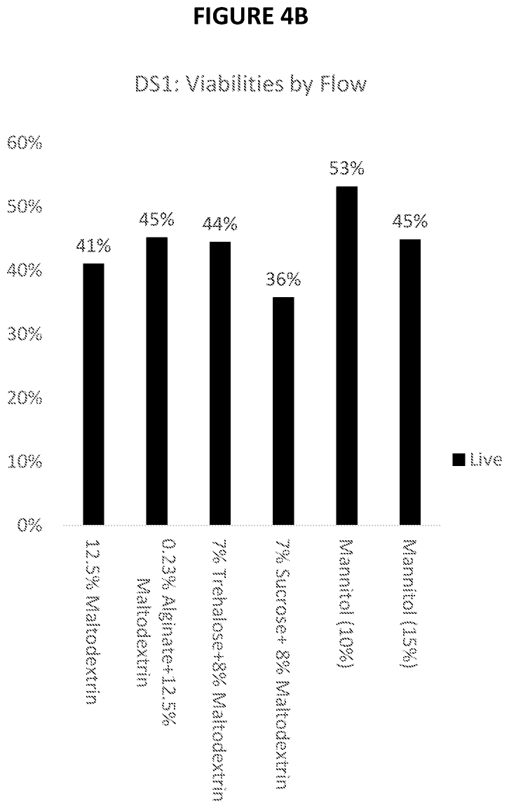

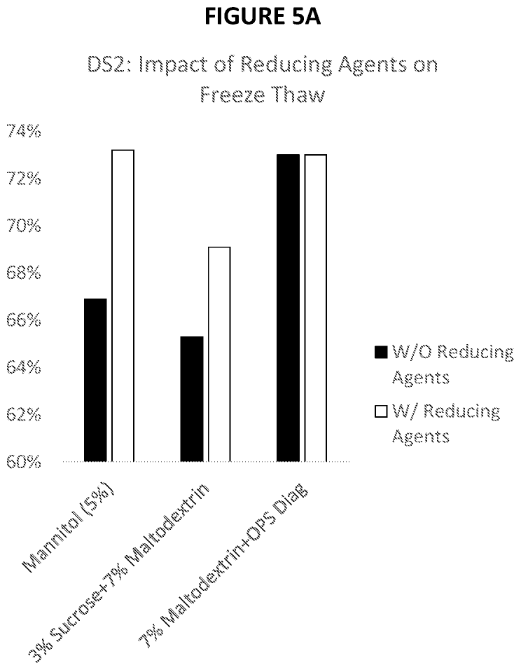

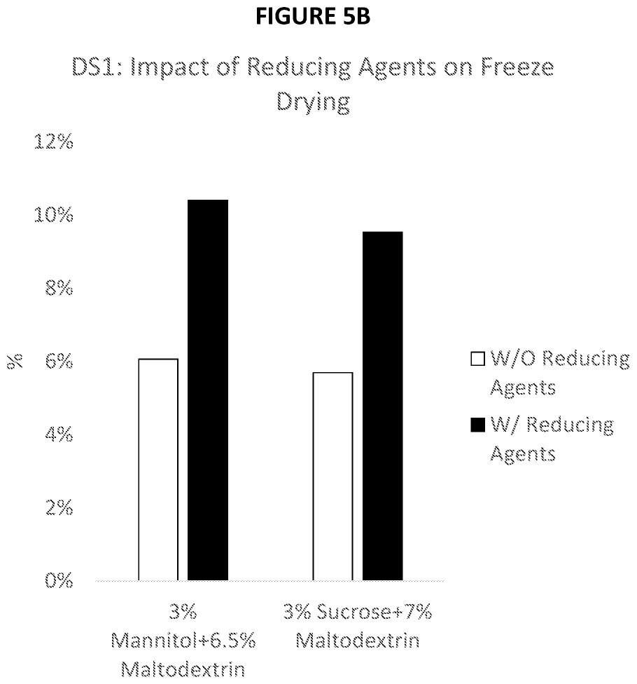

By means of a second example, it was necessary to determine the correct formulation for the lyophilization buffer/media. The following lyoprotectants were investigated to determine the correct formulation for each DS: sorbitol, maltodextrin, OPS diagnostics buffer, sucrose, inulin, alginate, mannitol, trehalose, and skim milk. For example, FIG. 4A shows examples of different viabilities of DS2 based on different lyoprotectants and FIG. 4B shows examples of different viabilities of DS1 based on different lyoprotectants. The addition of reducing agents including but not limited to cysteine HCL and riboflavin were also investigated as shown in FIG. 5A (DS2) and FIG. 5B (DS1). Additional lyophilization formulations that were tested include 8% Maltodextrin+0.5% Inulin+RA, 5% Sucrose+10% Glycerol+0.3% Inulin+RA, 7% Trehalose+8% Maltodextrin+RA, 3% Sucrose+5% Maltodextrin+0.5% Inulin+RA, 5% Maltodextrin+OPS Diag+0.5% Inulin+RA, and 5% maltodextrin+10% Glycerol+0.3% Inulin+RA.

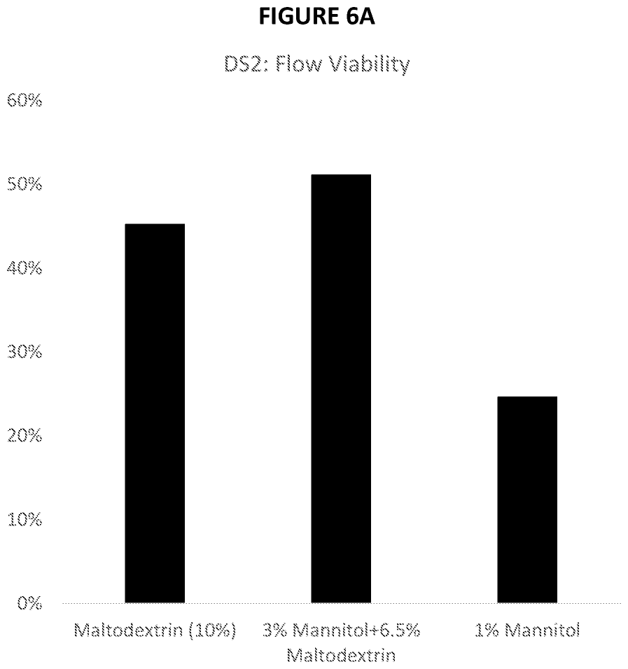

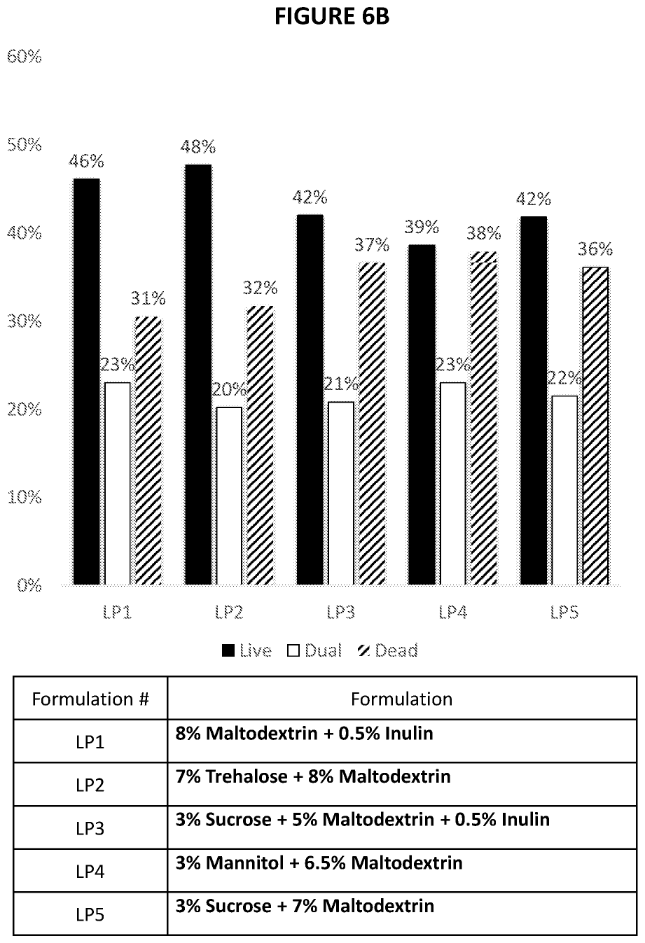

Based on freeze thaw and lyophilization experiments, data suggested that 10-12% solids was the selected dose. However, additional experiments were performed to determine if a lower dose would be possible. One exemplary experiment on DS2 is shown in FIG. 6A and a second exemplary experiment is shown in FIG. 6B.

Assays were then performed to determine the success rates of cell revival. Cell revival was done using the Anaerobe systems YCFAC media and dilution schemes were conducted using 100 fold dilution to the lyophilized powder (e.g., 50 mg (0.05 g) of powder was diluted in 5.0 mL of YCFAC media). Revival was then detected using flow cytometry and the Coulter Counter.

The experiments performed herein and the data generated determined that lyophilized material produced comparable colonization of strains in mice.

### Example 4: EH Mouse Models and Efficacy of Consortia

As described herein, enteric hyperoxaluria (EH) is caused by excess absorption of dietary oxalate leading to elevated urinary oxalate (UOx) levels. Once absorbed, oxalate can complex with calcium to form insoluble crystals, and as a result chronically elevated UOx levels are a major risk factor for the development of kidney stones and progression to kidney damage. There are currently no approved therapies for EH; the standard of care options is limited to supportive measures and dietary restrictions that have relatively low compliance. Most oxalate degradation in the human GI is carried out by Oxalobacter formigenes, a fastidious human commensal that metabolizes dietary oxalate as its primary energy source. However, it is hypothesized that increased antibiotic usage and western diets have decreased the prevalence of O. formigenes. Preliminary human studies have explored the therapeutic use of orally dosed O. formigenes and demonstrated limited engraftment of O. formigenes, leading to reduced durability of UOx reduction. Therefore, we reasoned that the metabolic support of a diverse consortia of GI commensals will enable engraftment of O. formigenes and maximum degradation of oxalate. To this end, microbial consortia were designed as described herein that mimic the taxonomic, phylogenetic, and functional structure of a healthy human microbiome. These consortia are not only enriched in O. formigenes to maximize oxalate metabolism but also contain numerous bacterial species to support the metabolism of formate, a byproduct of oxalate metabolism. A candidate was selected for clinical development in part by evaluating these consortia for their ability to engraft and reduce UOx in mouse models of diet-induced hyperoxaluria (HO).

Methods. Metagenomics and liquid chromatography-mass spectrophotometry (LC-MS) were used to evaluate bacterial species and urinary metabolites, respectively. Metagenomic sequencing was performed on select fecal samples from each study to evaluate O. formigenes engraftment, species richness, and community-specific strain level engraftment. LC-MS was used to evaluate levels of oxalate and creatinine from terminal spot urine samples collected.

Isolation and Processing. Isolation of bacterial strains to create synthetic consortia: bacterial strains to create consortia were isolated from healthy human stool samples collected under anaerobic conditions, homogenized, and then bacterial species from each sample were identified using whole-genome sequencing (WGS). From there, the bacterial strains and abundance thereof were identified.

Stool samples were then processed and bacterial strains isolated for culture on appropriate culture media (e.g. BHI, blood agar). Isolation of oxalate degrades and strains specific to metabolize EH-related pathways were prioritized along with fastidious and unique strains and strains associated with a healthy gut microbiome. Following culture, strains were purified and sequenced using metagenomics. From the cultured, isolated strains, communities to treat enteric hyperoxaluria were created based on the notion of our bacteria to fill critical functional niches in the gut, support normal GI physiology, support engraftment of specialty strains such as O. formigenes, and degrade oxalate.

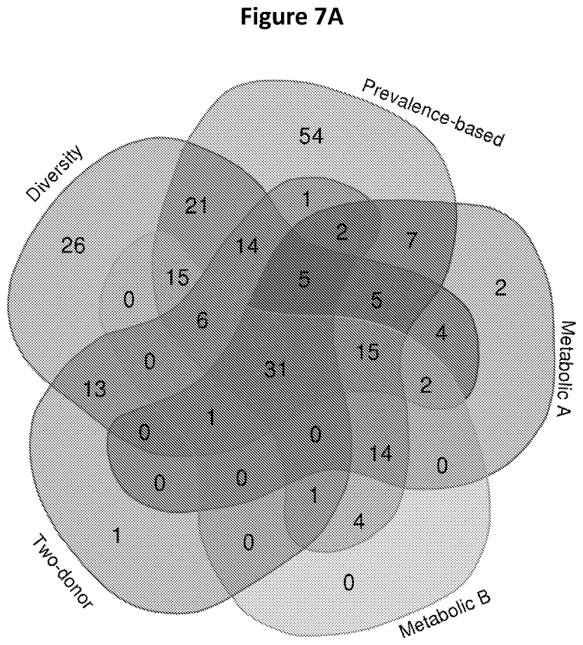

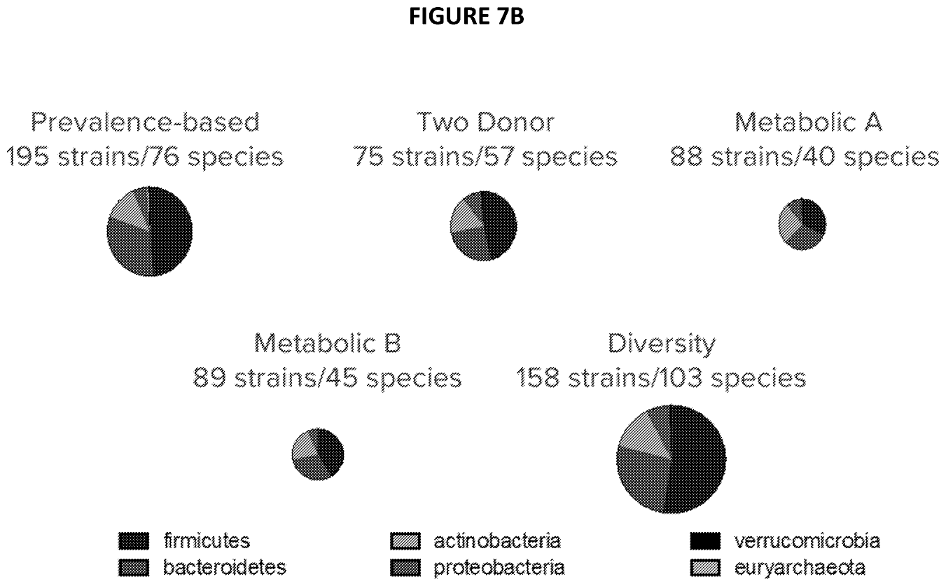

Diversity of synthetic consortia: consortia were created to support engraftment of O. formigenes in the GI and each consortium contains unique species and strains to cover various metabolic phenotypes (e.g. bile acid metabolism, short chain fatty acid synthesis, oxalate degradation). A core set of 31 bacterial strains were similar between synthetic consortia and each community had its unique signature as indicated in the Venn diagram. The number of species present in each consortium created ranged from 40 to 103 species and the number of strains ranged from 75 to 195 as shown FIGS. 7A and 7B. The species and strains comprised varying proportions of the phylum-level diversity where the Bacteroidetes to firmicutes ratio ranges from 51% to 96% indicating that the general composition varied.

EH Model Development. Diet induced EH mouse models were created. Dietary components for induction of EH: three diets (Ox36, 5021+0.875% oxalate in drinking water (DW), and 5010 1.51) were created to induce EH for three weeks in germ-free mice with different caloric intake and sodium oxalate. Diet 1 (Ox36): Fat (% kcal): 13.5, Carbohydrate (% kcal): 66.0, Protein (% kcal): 20.5, Fiber (%): 6.0, and Sodium Oxalate (g/kg): 3.7. Diet 2 (5021): Fat (% kcal): 23.7, Carbohydrate (% kcal): 53.2, Protein (% kcal): 23.1, Fiber (%): 3.7, and Sodium Oxalate (g/kg): in drinking water. Diet 3 (5010 1.51): Fat (% kcal): 15.0, Carbohydrate (% kcal): 54.3, Protein (% kcal): 30.6, Fiber (%): 4.2, and Sodium Oxalate (g/kg): 21.5.

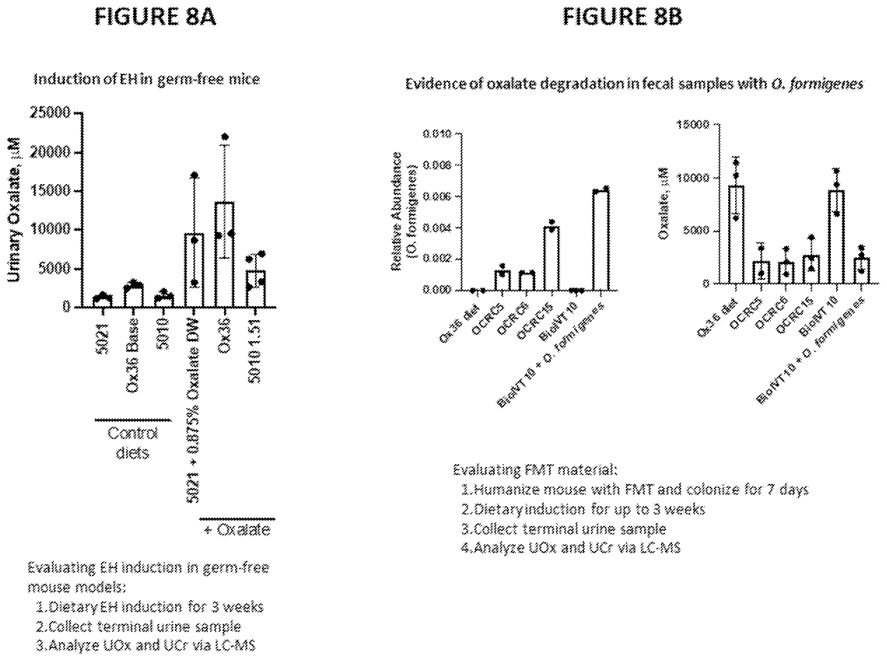

Induction of EH in germ-free and humanized mice: terminal urine samples were collected to measure UOx (urinary oxalate). BioIVT10 was identified as a possible FMT sample to develop and humanized, germ-free model of EH as the fecal sample was unable to control oxalate excretion and did not have O. formigenes present. This fecal sample showed that it could not degrade oxalate when colonized in a germ-free mouse and when supplemented with O. formigenes it was able to degrade oxalate. Additionally, this material showed no presence of O. formigenes. See FIGS. 8A and 8B.

Synthetic consortia reduce UOx and UOx:UCr ratio in EH-induced murine models: the three diets described above were tested in the development of microbial consortia to treat EH. All mice were dosed, via gavage, with 200 μL of each consortium on day 1. Two sets of mice were used: 1) Taconic germ-free C57BL/6NTac F (7-9 weeks old) that were Germ-Free, and 2) Taconic germ-free C57BL/6NTac F (7-9 weeks old) that were Humanized. For the Germ-Free mice, dietary EH induction began on D-7, consortia dosing began on D1, and the endpoint for feces and urine collection was on D15. For the Humanized mice, FMT was administered on D-21, dietary EH induction began on D-14, antibiotic treatment occurred on D-7, consortia dosing began on D1, and the endpoint for feces and urine collection was on D15. It was demonstrated that in using germ-free mice, a significant, 3-5 fold increase in urinary oxalate levels are observed across all diets. Furthermore, the 5010 1.51 diet was used in a humanization model where mice were colonized with an FMT. Three different FMT materials with and without O. formigenes were used and it was shown that an FMT that does not have O. formigenes present was unable to reduce oxalate degradation compared to control.

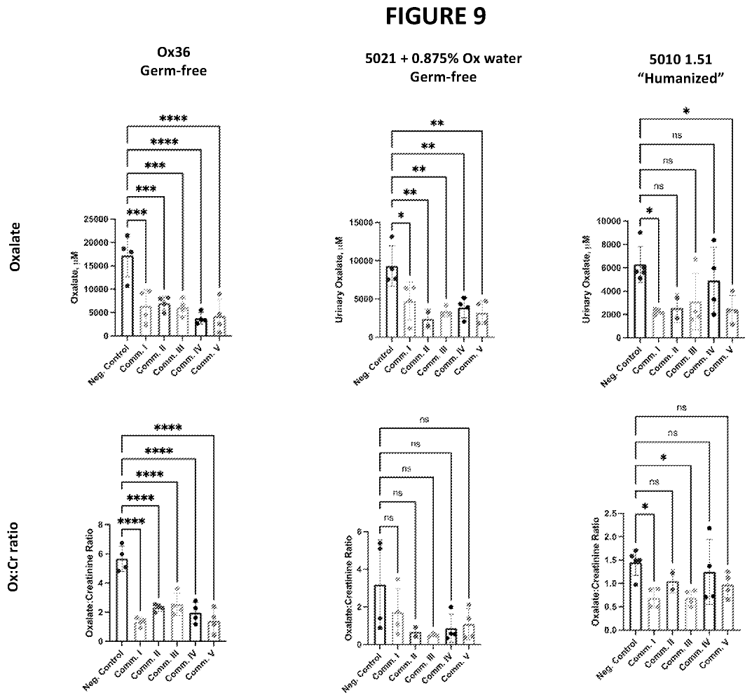

Synthetic consortia reduce UOx and UOx: UCr ratio in EH-induced murine models. After establishing that hyperoxaluria could be induced in germ-free mice, the question of whether or not oxalate excretion could be controlled with the administration of a consortia described herein. To do that, the germ free mice were induced for hyperoxaluria for 7 days by providing one of the three diets described above, given a single dose of one of the consortia, and then euthanized for terminal urine collection 14 days later. The diets were shown to effectively induce hyperoxaluria. On average, the consortia described herein reduced levels of oxalate in terminal urine samples collected. Because spot urine samples were collected, the oxalate to creatinine ratio was calculated as a more robust measure of EH and the Prevalence-based and Diversity Communities consistently reduced the UOx:UCr ratio across all diets. The average % reduction in UOx:UCr across consortia was between 40 to 55%. See FIG. 9.

As described above, humanized mice were also created by providing an FMT to a germ-free mouse using a stool sample that cannot degrade oxalate. These mice were provided a complex high oxalate diet and then were pre-treated with antibiotic to reduce the host microbiome. After a 1-week course of antibiotics, mice were dosed with one of the consortia described herein. The consortia described herein had varying degrees of oxalate reduction.

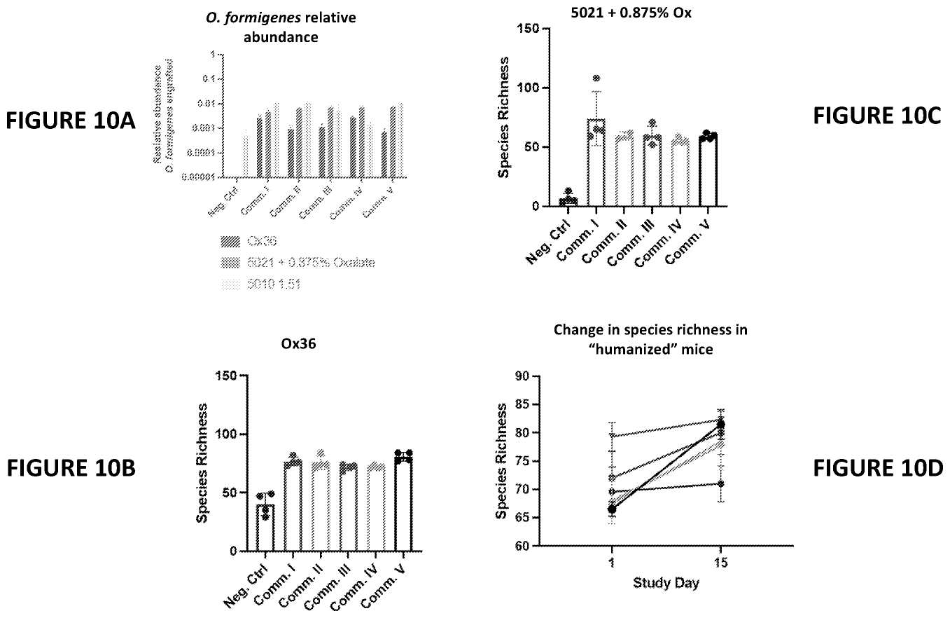

Consortia engraftment in various EH-induced models: the engraftment of O. formigenes and other consortia members were evaluated using metagenomic sequencing. O. formigenes engrafted to robust levels across all diets tested with Prevalence-based and Diversity Communities engrafting at the greatest relative abundance. Additionally, a greater proportion of strains and species in Prevalence-based and Diversity Communities engrafted to detectable levels as shown in species richness plots. Lastly, the Diversity Community had greater species richness compared to Five rationally-designed, synthetic consortia were created from donor fecal samples with varying degrees of diversity, fortified with O. formigenes, to control oxalate metabolism in the GI tractbaseline in the “humanized” model, indicating that in a complex model, the Diversity Community stably engrafts and displaces a previously established human community in germ-free mice. See, FIGS. 10A, 10B, 10C, and 10D.

Based on these experiments, it was determined that the five rationally-designed, synthetic consortia used in this experiment had varying degrees of diversity and were able to control oxalate metabolism in the GI tract to varying degrees. It was further shown that the diverse consortia described herein are able to engraft following dosing. Specifically, the experiments described herein show that O. formigenes was one of the microbes that were able to engraft. Furthermore, it was shown that the consortia described herein were able to reduce oxalate excretion (UOx and UOx:UCr ration) in dietary induced EH models to varying degrees and that the Community V, the consortium with the greatest diversity described in this Example 5, had the ability to stably engraft O. formigenes, therapeutically reduce UOx, and lead to a healthy human microbiome.

### Example 5: The Manufacture of Threonine Auxotrophic Microorganisms

Certain microorganisms are auxotrophs. This means that the microorganism is not able to synthesize a particular organic compound required for its growth. One such organic compound that certain microorganisms are incapable of synthesizing themselves is threonine. Furthermore, while some microorganisms are not per se auxotrophs of threonine, they are inefficient producers of threonine which prevent effective growth in commonly used growth medias.

N-Acetylgalactosamine (GalNAc) is an amino sugar derivative of galactose that is typically the first monosaccharide that connects serine or threonine in particular forms of protein O-glycosylation. While it is possible to supplement certain small batch growth medias with GalNAc to grow threonine auxotrophs without the addition of threonine, such supplementation is not preferred for large batch manufacture because GalNAc is costly and large amounts are needed for effective growth of microorganisms that require such galactose derivative. Furthermore, certain medias such as YCFAC media is incapable of effectively growing certain threonine auxotrophs even in the presence of GalNAc.

Accordingly, a method of improving the expansion and growth of inefficient producers of threonine is needed to effectively grow such microorganisms.

One such microorganism included in the consortia described herein is Akkermansia muciniphilia. Akkermansia is not capable of synthesizing threonine itself and thus is not able to effectively expand and grow in culture that is lacking a GalNAc source (or a primary source that can be metabolized into GalNAc). Furthermore, GalNAc is the preferred carbon source for Akkermansia and thus known methods of effectively growing and manufacturing Akkermansia comprise the addition of GalNAc to the growth media.

Accordingly, experiments were designed to identify novel methods of growing Akkermansia in large batches without large amounts of GalNAc. Specifically, three different growth medias were tested: YCFAC+GalNAc, YCFAC+GalNAc+Threonine, and YCFAC+Threonine. SinceBHIis an animal-based media that contains threonine, BHI media was used as a positive control (specifically BHI media+GalNAc+Hemin+VitaminK). Because GalNAc is the preferred carbon source for Akkermansia, it was expected to be needed in all medias in order to allow expansion and growth of the microorganism; however, the expected question was how much GalNAc is needed, not whether GalNAc was needed at all, if threonine is also added. Surprisingly, it was determined that 1) YCFAC+0.5 g/L GalNAc did not support Akkermansia growth, 2) YCFAC+0.5 g/L GalNAc+10 mM threonine did support growth, and that 3) YCFAC+10 mM threonine alone supports the growth of Akkermansia. In these experiments, a seed culture containing 0.5 g/L GalNAc in YCFAC was used to initiate cell growth before being transferred to large fermenter for growth and expansion with the 3 medias described above.

However, certain of the consortia described herein comprise more than 100 different microorganisms, Akkermansia being only one of the more than 100 different microorganisms. Furthermore, the manufacturing methods described herein allow for the growth and manufacturing of multiple microorganisms in a single large batch culture (e.g., in a fermenter). The question then became how to grow Akkermansia in a large co-culture when it is the only microorganism that is a threonine auxotroph that has a preferred carbon source of GalNAc. Accordingly, an experiment was designed to determine if it was possible to start a seed culture with Akkermansia alone and then combine it with a second seed culture of multiple microorganisms for the large batch expansion.

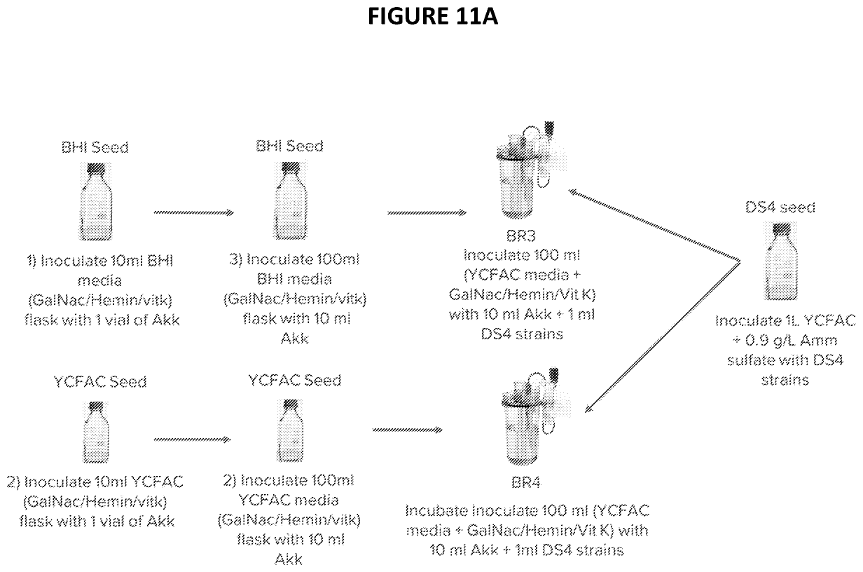

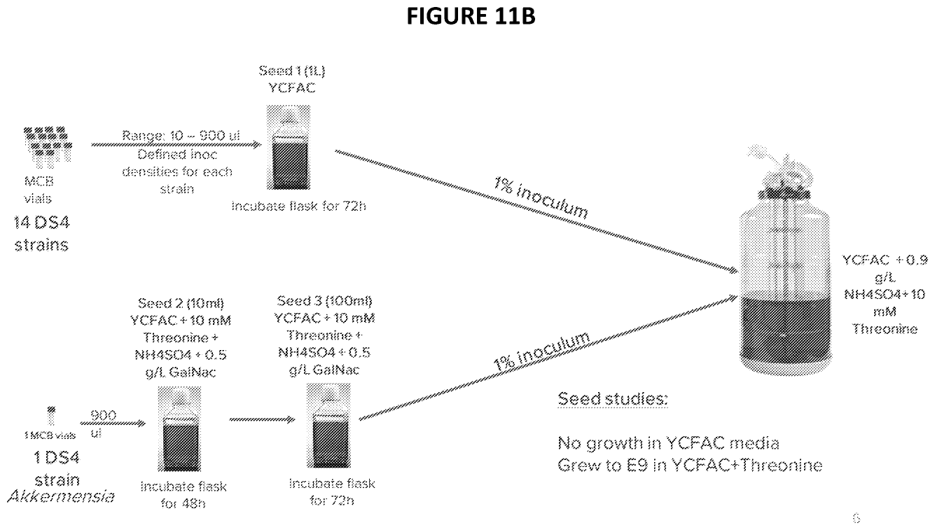

This experiment comprised: 1) a seed culture was first grown to allow the Akkermansia to begin growing in a small culture (i.e., a seed culture) of 10 mL before expansion into a large batch fermenter, 2) concurrently with the Akkermansia seed culture, a second 100 mL seed culture of all other microorganism in the drug substance was separately grown, 3) the 100 mL seed co-culture and the 10 mL Akkermansia seed culture were combined into a large batch fermenter (e.g., 1 L or more), and 4) the strains of the drug substance were detected and the ability of Akkermansia to grow and expand in the co-culture was assessed. A diagram of this experiment is shown in FIG. 11A.

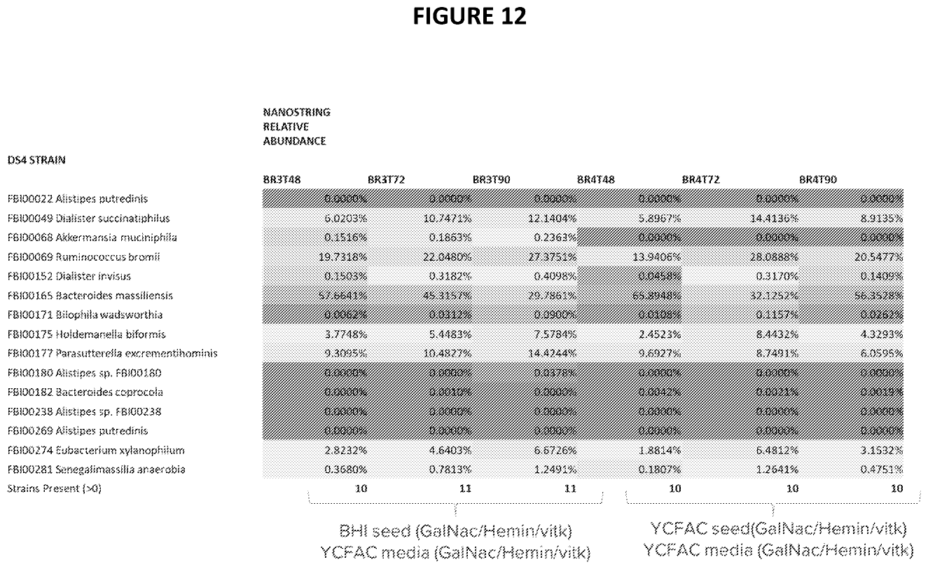

As shown in FIG. 12, it was surprising to see that Akkermansia was unable to grow in YCFAC media that was supplemented with GalNAc, Hemin, and VitaminK (0.0000% Akkermansia detected) compared to BHI media that was supplemented with GalNAc, Hemin, and VitaminK. Accordingly, it was determined that YCFAC+GalNAc cannot support the growth of Akkermansia. The question then became whether the addition of threonine could recover the growth of the Akkermansia.

The next question was whether GalNAc was needed for if threonine was added. Specifically, the question was how would Akkermansia grow in YCFAC+10 mM threonine (72 hr growth) compared to a media comprising YCFAC+10 mM threonine+0.5 g/L GalNAc (48 hr growth). It was surprising to find that the results showed comparable growth with and without the GalNAc (an OD of 0.25 for w/o GalNAc and an OD of 0.35 for w/GalNAc).

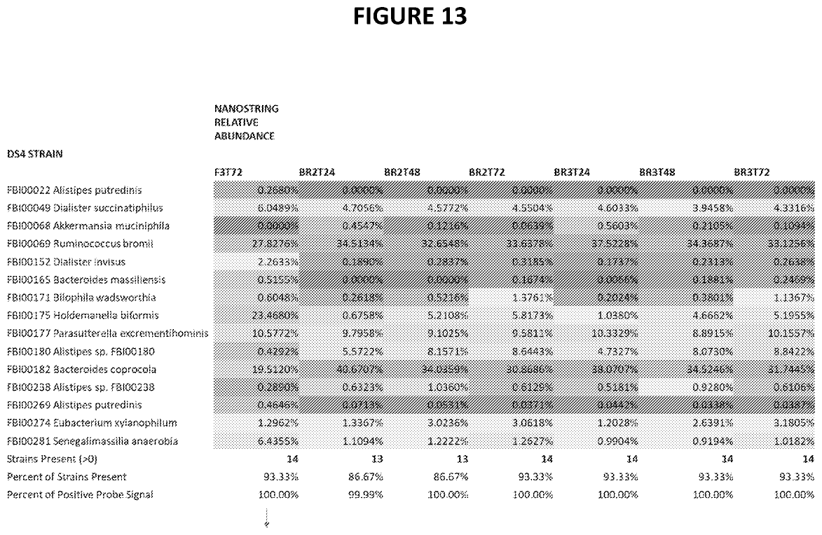

A co-culture experiment similar to that described above and shown in FIG. 11A was designed to evaluate the need for GalNAc and threonine. In this experiment, two seed cultures were used: 1) Akkermansia seed grown in YCFAC+10 mM threonine+0.5 g/L GalNAc, and 2) the other microorganisms in the drug substance (14 microorganisms) grown in YCFAC alone. The seed cultures were then combined into a large batch fermenter comprising YCFAC+10 mM threonine (i.e., no GalNAc). See FIG. 11B. This study showed that no GalNAc was needed in the presence of 10 mM threonine in a large batch fermenter in order for Akkermansia to grow in a co-culture with other microorganisms that are not threonine auxotrophs. Furthermore, in the 10 mM threonine YCFAC media, Akkermansia was detected at all growth time points (FIG. 13).

Additional experiments further showed that GalNAc was not even needed in the seed culture in order to achieve Akkermansia growth.

The ability to grow Akkermansia without GalNAc was very surprising given that GalNAc is Akkermansia's preferred carbon source. Furthermore, the ability to grow Akkermansia in a media without GalNAc provides a means of making microbial drug products comprising GalNAc wherein the Akkermansia is grown in a co-culture of multiple microbes.

### Example 6: Clinical Candidate Selection

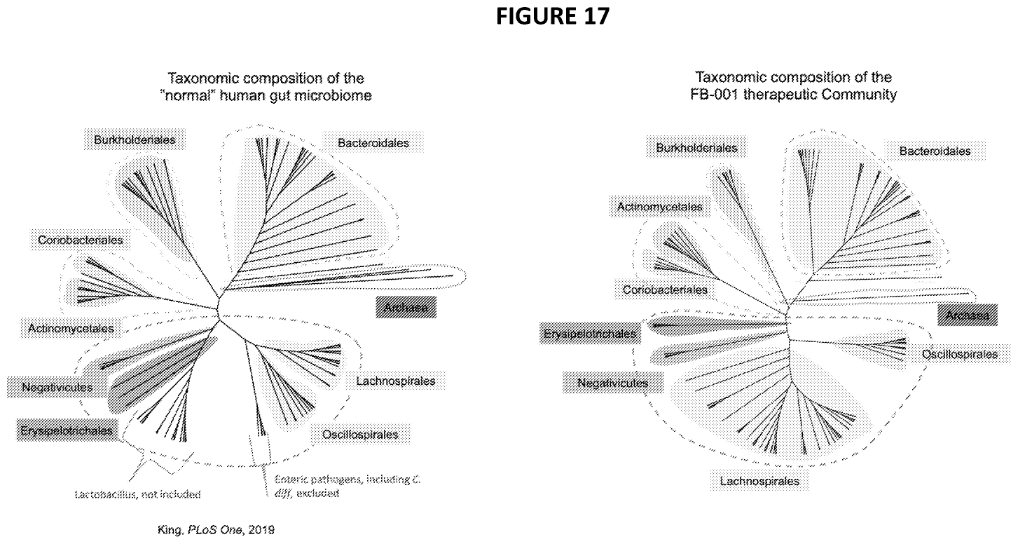

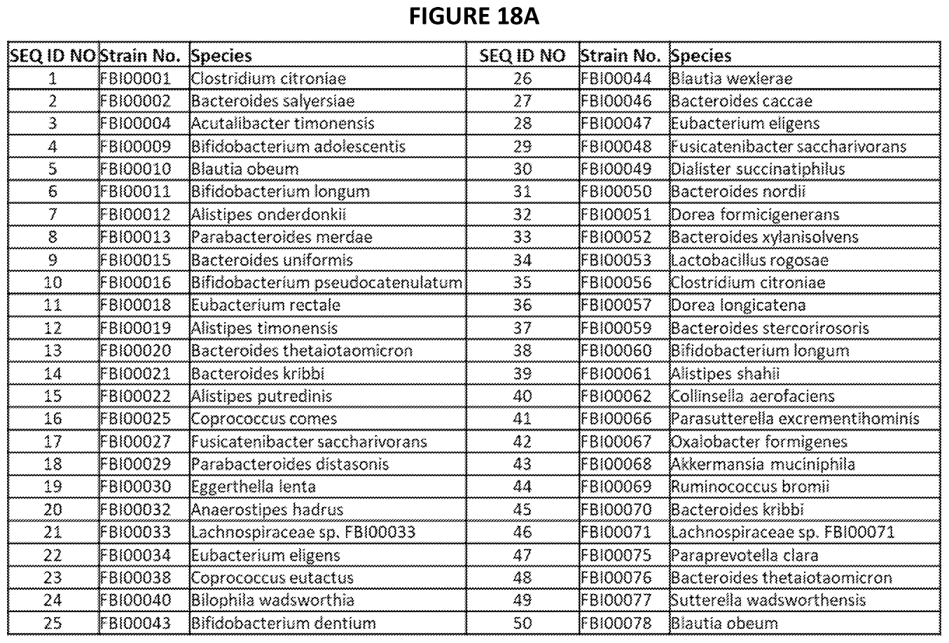

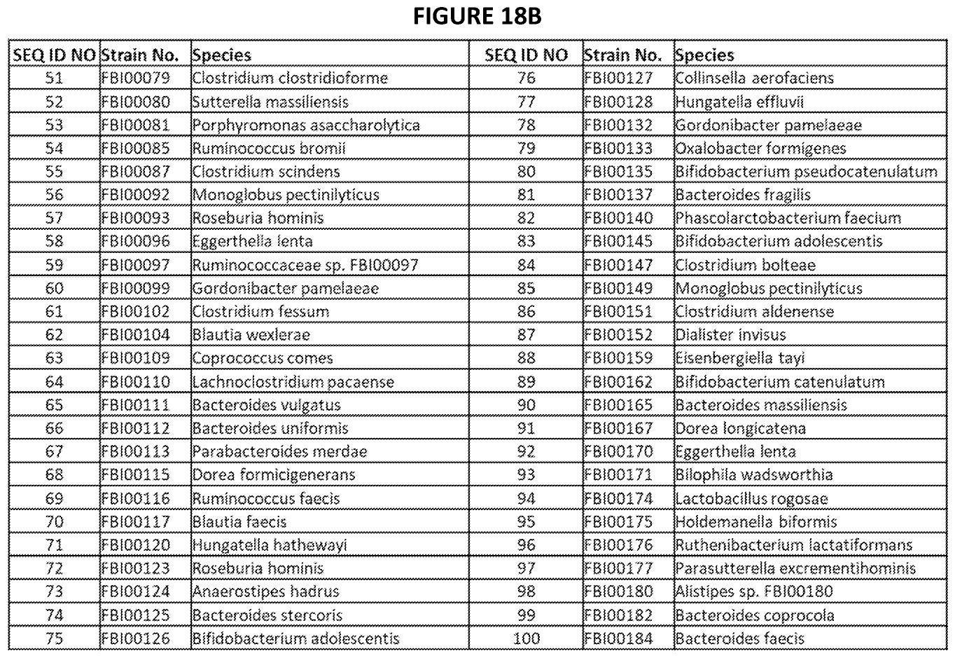

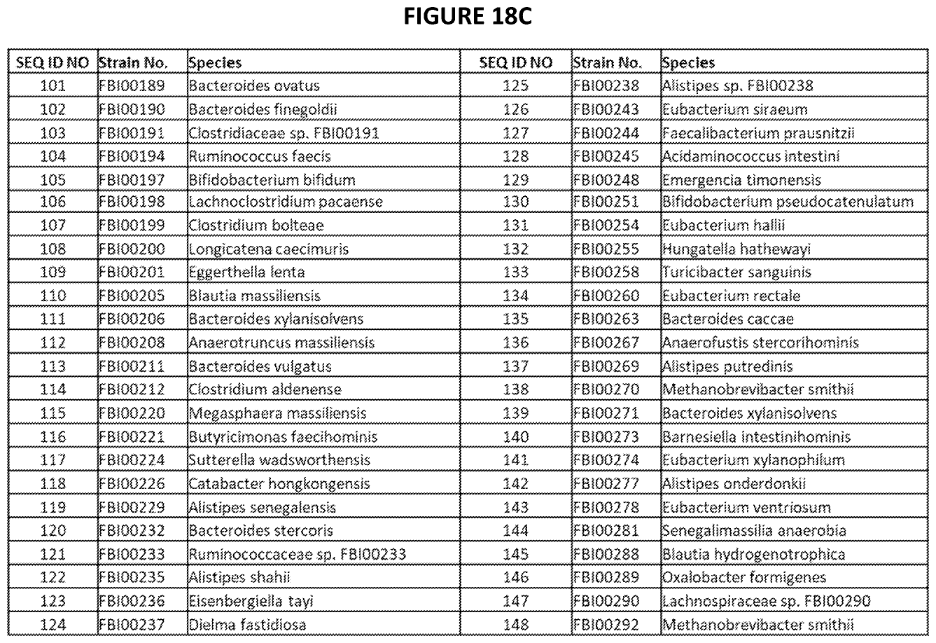

As described above, Consortia IX was selected as the clinical candidate for clinical trials and was termed FB-001. FB-001 comprises 148 different anaerobic microbial strains that was designed to emulate the metabolic and phylogenetic diversity of the human microbiome (FIG. 17) and was split into 7 different drug substances for manufacturing purposes. Table 22 shows the 7 different drug substances. Species were identified by 16S rRNA gene sequencing and whole genome sequencing of RCBs. The species in the consortium span six of the major phyla found in the GI tracts of healthy adults (King, Desai et al. 2019) with the deliberate exception of Fusobacteria, a phylum generally associated with human infections and enriched for opportunistic pathogens. The 148 strains encompass 10 distinct classes, 18 orders, 26 families, and 59 genera.

Prior to lyophilization, the cell pellet containing the FB-001 microbial strains was resuspended in YCFAC media with lyoprotectants and then lyophilized. The YCFAC media and lyoprotectants were chosen to stabilize the DS during the lyophilization step. The lyoprotectant combination of 8% maltodextrin+0.5% inulin was chosen for the final DS formulation as it demonstrated high viability of the FB-001 microbial strains in formulation development studies.

Maltodextrin was also added as a filler during DP manufacturing.

The capsules to encapsulate the DP were enteric coated and were chosen to release the DP in the small intestine and resist the gastric acids as they pass through the gastrointestinal tract. The dissolution of these capsules was tested per USP <701> at a pH of 1.2 and showed no disintegration for 2 hours. At a pH of 6.8, the capsules fully disintegrated within 30 minutes, which is the target release pH in the GI tract for FB-001 DP (Hydroxypropyl methylcellulose [HPMC] Capsule COA).

Function Properties of FB-001. FB-001 was manufactured using 7 individual drug substances (DS) that contain a total of 148 anaerobic microbial strains and is enriched for species performing beneficial or normalizing functions in the human GI tract.

The first of these beneficial or normalizing functions is oxalate degradation, which is the primary EH disease modifying mechanism of FB-001. Oxalobacter formigenes is the principal driver of oxalate degradation in the human GI tract. O. formigenes uses oxalate as its exclusive energy source, metabolizing significant concentrations of oxalate for energy generation and biomass production. The metabolism of oxalate is mediated by a series of enzymatic and transport reactions that ultimately consume oxalate and release CO₂ and formate.

Formate, as a by-product of oxalate metabolism, can ultimately inhibit further oxalate metabolism in vitro if it is not removed. Therefore, FB-001 also contains strains capable of formate degradation. These formate-utilizing bacteria help to clear the potentially inhibitory metabolic byproducts of oxalate metabolism.

FB-001 also contains strains that are oxalate resistant, able to grow in the presence of oxalate concentrations that are over a magnitude or higher than the physiologically normal concentrations of oxalate. This enrichment of oxalate-tolerant strains in the FB-001 consortium may support stable engraftment despite potentially elevated levels of free oxalate in the GI lumen of patients with EH, as the abundance of the key oxalotrophs will naturally increase with spikes in oxalate concentration.

The FB-001 consortium was specifically designed to contain phylogenetically diverse microbial species that function mutualistically to maximize the metabolic flux of oxalate (primary mechanism) and improve the dysbiosis associated with malabsorption (secondary mechanism). To ensure execution of both mechanisms, the FB-001 consortium is enriched for oxalate degrading strains to reduce free oxalate concentrations in the GI tract, as well as numerous species intended to support the community by restoring essential metabolic functions that reduce the malabsorption of any oxalate that is not degraded. The strains that make up the FB-001 consortium were selected based on their predicted ability to perform a variety of supportive metabolic functions that would contribute to engraftment regardless of differences in patient physiology or diet. Metabolism of macronutrients and dietary molecules that are not digested or utilized by host cells may result in the release of metabolic products that feed other members of the microbiome community.

Other strains in FB-001 were evaluated for unique and potentially beneficial biological functions in the GI tract, including production of short-chain fatty acids (SCFAs), cross-feeding activity, and mucin degradation. SCFAs are absorbed by the host and have been recognized to confer a range of health-promoting functions by acting as key energy substrates for colonocytes, enterocytes, and hepatocytes, while also acting as signaling molecules recognized by specific G-protein couple receptors targeting primarily enteroendocrine and immune cells in the lamina propria of the intestinal mucosa. Strains in FB-001 were evaluated for their cross-feeding activity, a process in which bacteria make by-products that feed other bacteria. Cross-feeding stabilizes the gut microbiome and creates novels niches. Strains in FB-001 were also evaluated for putative protective and/or anti-inflammatory properties.

Table 23 summarizes the number of strains in FB-001 that contribute to each of these functional properties, and characteristics that are associated with each FB-001 species are summarized in Table 24.

TABLE 23
Function Properties of FB-001 DP

| | | Number of |
|---|---|---|
| | | FB-001 |
| Properties | Classification | DP Strains |
| Oxalate and formate | Oxalate degradation | 7 |
| metabolism | Oxalate resistance | 38 |
| | Formate metabolism | 45 |
| Supportive metabolic | Metabolism of macronutrients | 98 |
| functions | Production of microbial metabolites | 70 |
| | Production of short-chain fatty acids | 131 |
| | Cross-feeding activity | 12 |
| | Mucin degradation | 4 |

| | |
|---|---|
| Putative protection against disease | 22 |
| Prevalence in healthy human gut | 97 |

TABLE 24
Species Included in FB-001 Drug Product and Characteristics

| | | | | | | Production |
|---|---|---|---|---|---|---|
| | # | Oxalate | Oxalate | Formate | Metabolism of | of Microbial |
| Species (by phylum) | strains | Degradation | Resistance | Metabolism | Macronutrients | Metabolites |
| Actinobacteria | | | | | | |
| Bifidobacterium adolescentis | 3 | | | | ● | ● |
| Bifidobacterium bifidum | 1 | | | | ● | |
| Bifidobacterium catenulatum | 1 | | | | ● | ● |
| Bifidobacterium dentium | 1 | ● | | | ● | ● |
| Bifidobacterium longum | 2 | | | | ● | ● |
| Bifidobacterium pseudocatenulatum | 3 | ● | | | ● | ● |
| Collinsella aerofaciens | 2 | | | ● | ● | ● |
| Eggerthella lenta | 4 | | ● | ● | ● | |
| Gordonibacter pamelaeae | 2 | | | ● | ● | |
| Senegalimassilia anaerobia | 1 | | | ● | | |
| Bacteroidetes | | | | | | |
| Alistipes onderdonkii | 2 | | | | ● | ● |
| Alistipes putredinis | 2 | | | | | ● |
| Alistipes senegalensis | 1 | | | | ● | |
| Alistipes shahii | 2 | | | | ● | |
| Alistipes timonensis | 1 | | | ● | ● | |
| Alistipes sp. FBI00180 | 1 | | | | | |
| Alistipes sp. FBI00238 | 1 | | | | | |
| Bacteroides caccae | 2 | | | | ● | |
| Bacteroides coprocola | 1 | | | | | |
| Bacteroides faecis | 1 | | | ● | | ● |
| Bacteroides finegoldii | 1 | | | ● | | |
| Bacteroides fragilis | 1 | | | | ● | |
| Bacteroides kribbi | 2 | | | ● | ● | |
| Bacteroides massiliensis | 1 | | | | ● | |
| Bacteroides nordii | 1 | | | | | |
| Bacteroides ovatus | 1 | | | ● | ● | |
| Bacteroides salyersiae | 1 | | | ● | | |
| Bacteroides stercorirosoris | 1 | | | ● | | |
| Bacteroides stercoris | 2 | | | | ● | ● |
| Bacteroides thetaiotaomicron | 2 | | ● | ● | ● | ● |
| Bacteroides uniformis | 2 | | | | ● | |
| Bacteroides vulgatus | 2 | | | ● | ● | ● |
| Bacteroides xylanisolvens | 3 | | ● | ● | ● | ● |
| Barnesiella intestinihominis | 1 | | | | | |
| Butyricimonas faecihominis | 1 | | | | ● | ● |
| Parabacteroides distasonis | 1 | | ● | | ● | ● |
| Parabacteroides merdae | 2 | | | | ● | |
| Paraprevotella clara | 1 | | | ● | ● | ● |
| Porphyromonas asaccharolytica | 1 | | | | | |
| Euryarchaeota | | | | | | |
| Methanobrevibacter smithii | 2 | | | ● | ● | ● |
| Acidaminococcus intestini | 1 | | | | | ● |
| Acutalibacter timonensis | 1 | | ● | | | |
| Anaerofustis stercorihominis | 1 | | ● | | ● | ● |
| Anaerostipes hadrus | 2 | | | | ● | ● |
| Anaerotruncus massiliensis | 1 | | | ● | ● | |
| Blautia faecis | 1 | | ● | | | ● |
| Blautia hydrogenotrophica | 1 | | | ● | | ● |
| Blautia massiliensis | 1 | | | ● | | ● |
| Blautia obeum | 2 | | ● | | ● | ● |
| Blautia wexlerae | 2 | | | ● | ● | ● |
| Catabacter hongkongensis | 1 | | ● | | ● | |
| Clostridiaceae sp. FBI00191 | 1 | | | | | |
| Clostridium aldenense | 2 | | ● | | ● | |
| Clostridium bolteae | 2 | | ● | ● | ● | |
| Clostridium citroniae | 2 | | ● | | ● | |
| Clostridium clostridioforme | 1 | | | | | |
| Clostridium fessum | 1 | | | | | |
| Clostridium scindens | 1 | | | | ● | |
| Coprococcus comes | 2 | | | | ● | ● |
| Coprococcus eutactus | 1 | | | | | |
| Dialister invisus | 1 | | | | | |
| Dialister succinatiphilus | 1 | | ● | | ● | ● |
| Dielma fastidiosa | 1 | | | | ● | |
| Dorea formicigenerans | 2 | | | ● | ● | |
| Dorea longicatena | 2 | | | | ● | |
| Eisenbergiella tayi | 2 | | | | | ● |
| Emergencia timonensis | 1 | | | | ● | ● |
| Eubacterium eligens | 2 | | | | ● | |
| Eubacterium hallii | 1 | | | | ● | ● |
| Eubacterium rectale | 2 | | | | ● | |
| Eubacterium siraeum | 1 | | | | ● | |
| Eubacterium ventriosum | 1 | | | | ● | |
| Eubacterium xylanophilum | 1 | | | | ● | ● |
| Faecalibacterium prausnitzii | 1 | | | ● | ● | ● |
| Fusicatenibacter saccharivorans | 2 | | | | | ● |
| Holdemanella biformis | 1 | | | | | ● |
| Hungatella effluvii | 1 | | | | | |
| Hungatella hathewayi | 2 | | | | ● | ● |
| Lachnoclostridium pacaense | 2 | | | | | |
| Lachnospiraceae sp. FBI00033 | 1 | | | | | |
| Lachnospiraceae sp. FBI00071 | 1 | | | | | |
| Lachnospiraceae sp. FBI00290 | 1 | | | | | |
| Lactobacillus rogosae | 2 | | | | | |
| Longicatena caecimuris | 1 | | | | | |
| Megasphaera massiliensis | 1 | | | ● | | ● |
| Monoglobus pectinilyticus | 2 | | ● | | ● | ● |
| Phascolarctobacterium faecium | 1 | | ● | | | ● |
| Roseburia hominis | 2 | | ● | | ● | ● |
| Ruminococcaceae sp. FBI00097 | 1 | | | | | ● |
| Ruminococcaceae sp. FB100233 | 1 | | | | | ● |
| Ruminococcus bromii | 2 | | ● | | ● | |
| Ruminococcus faecis | 2 | | ● | | ● | ● |
| Ruthenibacterium lactatiformans | 1 | | | | ● | ● |
| Turicibacter sanguinis | 1 | | | | | |
| Proteobacteria | | | | | | |
| Bilophila wadsworthia | 2 | | | ● | ● | |
| Oxalobacter formigenes | 3 | ● | ● | | | |
| Parasutterella excrementihominis | 2 | | | ● | | ● |
| Sutterella massiliensis | 1 | | ● | ● | | ● |
| Sutterella wadsworthensis | 2 | | ● | ● | | |
| Verrucomicrobia | | | | | | |
| Akkermansia muciniphila | 1 | | | | ● | |

| | | | | Putative | Known |
|---|---|---|---|---|---|
| | Production of | Cross- | | Protection | Prevalence |
| | Short-Chain | Feeding | Mucin | Against | in Healthy |
| Species (by phylum) | Fatty Acids | Activity | Degradation | Disease | Human Gut |
| Actinobacteria | | | | | |
| Bifidobacterium adolescentis | ● | | | | ● |
| Bifidobacterium bifidum | | ● | ● | | ● |
| Bifidobacterium catenulatum | ● | | | | ● |
| Bifidobacterium dentium | ● | | | | ● |
| Bifidobacterium longum | ● | | | ● | ● |
| Bifidobacterium pseudocatenulatum | ● | | | | ● |
| Collinsella aerofaciens | ● | | | | ● |
| Eggerthella lenta | ● | | | | ● |
| Gordonibacter pamelaeae | ● | | | | ● |
| Senegalimassilia anaerobia | | | | | |
| Bacteroidetes | | | | | |
| Alistipes onderdonkii | ● | | | ● | ● |
| Alistipes putredinis | ● | | | | ● |
| Alistipes senegalensis | | | | | ● |
| Alistipes shahii | ● | | | ● | ● |
| Alistipes timonensis | ● | | | | ● |
| Alistipes sp. FBI00180 | ● | | ● | | |
| Alistipes sp. FBI00238 | ● | | | | |
| Bacteroides caccae | ● | | | ● | ● |
| Bacteroides coprocola | | | | | ● |
| Bacteroides faecis | ● | | | | ● |
| Bacteroides finegoldii | | | | | ● |
| Bacteroides fragilis | ● | | | | ● |
| Bacteroides kribbi | ● | | | | |
| Bacteroides massiliensis | ● | | | | ● |
| Bacteroides nordii | ● | | | | ● |
| Bacteroides ovatus | | | | ● | ● |
| Bacteroides salyersiae | ● | | | | ● |
| Bacteroides stercorirosoris | ● | | | | |
| Bacteroides stercoris | ● | | | | ● |
| Bacteroides thetaiotaomicron | ● | ● | | | ● |
| Bacteroides uniformis | ● | ● | | | ● |
| Bacteroides vulgatus | ● | | | | ● |
| Bacteroides xylanisolvens | ● | | | | ● |
| Barnesiella intestinihominis | | | | ● | ● |
| Butyricimonas faecihominis | ● | | | | |
| Parabacteroides distasonis | ● | | | | ● |
| Parabacteroides merdae | ● | | | | ● |
| Paraprevotella clara | ● | | | | ● |
| Porphyromonas asaccharolytica | ● | | | | ● |
| Euryarchaeota | | | | | |
| Methanobrevibacter smithii | ● | | | | ● |
| Acidaminococcus intestini | ● | | | | ● |
| Acutalibacter timonensis | ● | | | | |
| Anaerofustis stercorihominis | ● | | | | ● |
| Anaerostipes hadrus | ● | ● | | | ● |
| Anaerotruncus massiliensis | | | | | ● |
| Blautia faecis | ● | | | | ● |
| Blautia hydrogenotrophica | ● | | | | ● |
| Blautia massiliensis | ● | | | | |
| Blautia obeum | ● | | | ● | ● |
| Blautia wexlerae | ● | | | ● | |
| Catabacter hongkongensis | | | | | ● |
| Clostridiaceae sp. FBI00191 | ● | | | | |
| Clostridium aldenense | ● | | | | ● |
| Clostridium bolteae | ● | | | | ● |
| Clostridium citroniae | ● | | | | ● |
| Clostridium clostridioforme | ● | | | | ● |
| Clostridium fessum | ● | | | | |
| Clostridium scindens | ● | | | | |
| Coprococcus comes | ● | | | | ● |
| Coprococcus eutactus | ● | | | | ● |
| Dialister invisus | ● | | | | ● |
| Dialister succinatiphilus | ● | | | | |
| Dielma fastidiosa | | | | | |
| Dorea formicigenerans | ● | | | | ● |
| Dorea longicatena | ● | | | | ● |
| Eisenbergiella tayi | ● | | | | |
| Emergencia timonensis | | | | | |
| Eubacterium eligens | ● | | | ● | |
| Eubacterium hallii | ● | | | | ● |
| Eubacterium rectale | ● | ● | | | ● |
| Eubacterium siraeum | | | | | |
| Eubacterium ventriosum | | | | ● | ● |
| Eubacterium xylanophilum | ● | | | | ● |
| Faecalibacterium prausnitzii | ● | | ● | ● | ● |
| Fusicatenibacter saccharivorans | ● | | | | |
| Holdemanella biformis | | | | ● | ● |
| Hungatella effluvii | ● | | | | |
| Hungatella hathewayi | ● | | | | |
| Lachnoclostridium pacaense | ● | | | | |
| Lachnospiraceae sp. FBI00033 | ● | | | | |
| Lachnospiraceae sp. FBI00071 | ● | | | | |
| Lachnospiraceae sp. FBI00290 | ● | | | | |
| Lactobacillus rogosae | ● | | | | |
| Longicatena caecimuris | ● | | | | |
| Megasphaera massiliensis | ● | | | | |
| Monoglobus pectinilyticus | ● | | | | |
| Phascolarctobacterium faecium | ● | | | | |
| Roseburia hominis | ● | | | ● | ● |
| Ruminococcaceae sp. FBI00097 | ● | | | | |
| Ruminococcaceae sp. FB100233 | ● | | | | |
| Ruminococcus bromii | ● | ● | | | ● |
| Ruminococcus faecis | ● | | | | |
| Ruthenibacterium lactatiformans | ● | | | | |
| Turicibacter sanguinis | ● | | | ● | |
| Proteobacteria | | | | | |
| Bilophila wadsworthia | ● | | | | |
| Oxalobacter formigenes | | | | | |
| Parasutterella excrementihominis | ● | | | | ● |
| Sutterella massiliensis | ● | | | | |
| Sutterella wadsworthensis | ● | | | | ● |
| Verrucomicrobia | | | | | |
| Akkermansia muciniphila | ● | ● | ● | | ● |

Formate Metabolism. The FB-001 DP consortium also contains formate-utilizing bacteria to maintain maximal carbon flux through the pathway. Formate, as a by-product of oxalate metabolism, can ultimately inhibit further oxalate metabolism in vitro if it is not removed. Symbiotic bacterial species such as methanogens found in the human GI tract can efficiently remove formate via reduction to methane in the presence of hydrogen gas produced by microbial fermenters. Therefore, the FB-001 Consortia includes Methanobrevibacter smithii (DS-CoC2), the most prevalent and abundant archaeal methanogen in the gut, and one that efficiently metabolizes formate, as well as the acetogenic gut commensal Blautia hydrogenotrophica (DS-CoC1), which utilizes formate to generate acetate for short-chain fatty acid (SCFA) synthesis, and a panel of anaerobes (eg, Sutterella and Parasutterella, found in DS-CoC2 and DS-CoC4) that express cytochrome-dependent formate dehydrogenases that oxidize formate to CO₂. These formate-utilizing bacteria therefore help to clear the potentially inhibitory metabolic byproducts of oxalate metabolism.

Supportive Metabolic Functions. FB-001 also contains a diverse panel of broadly functional commensals that fulfill unique and potentially beneficial biological functions in the GI tract, including metabolism of macro-nutrients, production of short-chain fatty acids, cross-feeding activity, and mucin degradation.

Composition of FB-001 DP. FB-001 DP is a highly complex, mixed fermentation of 148 microbial strains, chosen for their potential role in supporting a healthy GI tract. To support clinical studies, FB-001 DP was characterized for relative abundance of individual species in the final DP using metagenomic sequencing, as well as for total O. formigenes content. In metagenomic sequencing and analysis, strains were first confirmed to be present in the sample by positive identification of pre-specified biomarkers (short sequences of DNA) that are unique to the strain of interest. Then, the results of metagenomic sequencing were reported as the relative abundance of each strain, which approximates the percentage of genome copies that belong to each strain and can range from 0 to 100%. The relative abundance was then calculated by comparing the number and frequency of detected biomarkers to the total number of strain-specific biomarkers and the number of sequencing reads. The percent contribution of each strain in the FB-001 DP comprises a predominant portion of the three O. formigenes strains identified by 16S RNA and carbon source analysis described below as follows: approximately 32% O. formigenes on a relative abundance basis (i.e., approximately 40% on a viable cell count basis) with the other 145 strains having relative abundance values ranging from 18% to 0.015% (distribution of a typical human microbiome).

FB-001 DP was manufactured as a single batch. A single capsule of DP from was collected and stored at −20° C.±5 until DNA extraction. FB-001 DP was sequenced via shotgun metagenomics and the metagenomic sequences of DP were analyzed to determine the composition of FB-001 DP. Results were reported as the relative abundance of each strain. Relative abundance approximates the percentage of FB-001 DP genome copies that belong to each strain and can range from 0 to 100%. A total of 60 of 148 strains were detected at or above their qualified limit of detection, including 21 strains from DS-CoC1, 13 strains from DS-CoC2, 16 strains from DS-CoC3, 7 strains from DS-CoC4, and each of DS-OF1, DS-OF2, and DS-OF3. The absence of detection of a strain should not be interpreted as its absence from the drug substance. The 60 detected strains account for 95.932% of the biomarkers detected in FB-001 DP. The remaining 88 strains therefore account for 4.068% of the biomarkers. The relative abundance profile is expected to vary between batches and data will continue to be collected during development to understand the magnitude of the variability. Furthermore, the exact percentages should not be interpreted as limiting or exclusive; rather each batch of DP may vary in its microbial distribution based on natural growth of bacterial in co-cultures. An example of the relative abundance profile of the microbes in one lot of FB-001 is provided in Table 25.

Table 25. Relative Abundance Profile of a FB-001 DP Lot

TABLE 25
Relative Abundance Profile of a FB-001 DP Lot

| | Rel- | | Rel- | | Rel- |
|---|---|---|---|---|---|
| | ative | | ative | | ative |
| | Abun- | | Abun- | | Abun- |
| | dance | | dance | | dance |
| Strain | (%) | Strain | (%) | Strain | (%) |
| FBI00180 | 18 | FBI00245 | 0.076 | FBI00097 | <LoD |
| FBI00289 | 11 | FBI00043 | 0.063 | FBI00099 | <LoD |
| FBI00067 | 11 | FBI00237 | 0.059 | FBI00109 | <LoD |
| FBI00133 | 10 | FBI00206 | 0.052 | FBI00113 | <LoD |
| FBI00038 | 4.3 | FBI00032 | 0.04 | FBI00115 | <LoD |
| FBI00175 | 4.1 | FBI00243 | 0.038 | FBI00117 | <LoD |
| FBI00255 | 3.9 | FBI00116 | 0.035 | FBI00123 | <LoD |
| FBI00120 | 3.3 | FBI00002 | 0.032 | FBI00126 | <LoD |
| FBI00177 | 2.5 | FB100167 | 0.031 | FBI00127 | <LoD |
| FBI00212 | 2.4 | FBI00068 | 0.015 | FBI00132 | <LoD |
| FBI00025 | 2.2 | FBI00010 | <LoD | FBI00137 | <LoD |
| FBI00060 | 1.8 | FB100011 | <LoD | FBI00145 | <LoD |
| FBI00048 | 1.6 | FB100012 | <LoD | FBI00149 | <LoD |
| FBI00104 | 1.5 | FBI00013 | <LoD | FBI00159 | <LoD |
| FBI00151 | 1.5 | FBI00015 | <LoD | FBI00162 | <LoD |
| FBI00220 | 1.1 | FBI00018 | <LoD | FBI00165 | <LoD |
| FBI00004 | 1 | FBI00019 | <LoD | FBI00170 | <LoD |
| FBI00020 | 0.93 | FBI00021 | <LoD | FBI00174 | <LoD |
| FBI00251 | 0.91 | FBI00022 | <LoD | FBI00182 | <LoD |
| FBI00016 | 0.73 | FBI00030 | <LoD | FBI00184 | <LoD |
| FBI00079 | 0.72 | FBI00033 | <LoD | FBI00189 | <LoD |
| FBI00102 | 0.71 | FBI00034 | <LoD | FBI00190 | <LoD |
| FBI00233 | 0.65 | FBI00040 | <LoD | FBI00191 | <LoD |
| FBI00171 | 0.64 | FBI00044 | <LoD | FBI00194 | <LoD |
| FBI00029 | 0.63 | FBI00046 | <LoD | FBI00200 | <LoD |
| FBI00076 | 0.56 | FBI00047 | <LoD | FBI00201 | <LoD |
| FBI00147 | 0.53 | FBI00049 | <LoD | FBI00208 | <LoD |
| FBI00128 | 0.5 | FBI00050 | <LoD | FBI00221 | <LoD |
| FBI00197 | 0.49 | FBI00051 | <LoD | FBI00224 | <LoD |
| FBI00110 | 0.47 | FBI00052 | <LoD | FBI00229 | <LoD |
| FBI00112 | 0.47 | FBI00053 | <LoD | FBI00235 | <LoD |
| FBI00135 | 0.47 | FBI00056 | <LoD | FBI00236 | <LoD |
| FBI00226 | 0.44 | FBI00057 | <LoD | FBI00238 | <LoD |
| FBI00199 | 0.43 | FBI00059 | <LoD | FBI00244 | <LoD |
| FBI00152 | 0.43 | FBI00061 | <LoD | FBI00248 | <LoD |
| FBI00027 | 0.42 | FBI00066 | <LoD | FBI00254 | <LoD |
| FBI00211 | 0.4 | FBI00069 | <LoD | FBI00258 | <LoD |
| FBI00232 | 0.37 | FBI00070 | <LoD | FBI00260 | <LoD |
| FBI00111 | 0.31 | FBI00071 | <LoD | FBI00267 | <LoD |
| FBI00140 | 0.3 | FBI00075 | <LoD | FBI00269 | <LoD |
| FBI00263 | 0.25 | FBI00077 | <LoD | FBI00270 | <LoD |
| FBI00124 | 0.24 | FBI00078 | <LoD | FBI00271 | <LoD |
| FBI00001 | 0.23 | FBI00080 | <LoD | FBI00273 | <LoD |
| FBI00198 | 0.21 | FBI00081 | <LoD | FBI00274 | <LoD |
| FBI00176 | 0.2 | FBI00085 | <LoD | FBI00277 | <LoD |
| FBI00125 | 0.2 | FBI00087 | <LoD | FBI00278 | <LoD |
| FBI00205 | 0.17 | FBI00092 | <LoD | FBI00288 | <LoD |
| FBI00062 | 0.096 | FBI00093 | <LoD | FBI00290 | <LoD |
| FBI00281 | 0.095 | FBI00096 | <LoD | FBI00292 | <LoD |
| FBI00009 | 0.09 | | | | |

Process Development. The blending process during DP manufacture was developed to create a homogenous mixture of the DSs. During the development phase, the blend-sieve-blend technique for mixing the DSs was tested. Using this technique, several of the DSs were blended in a Turbula mixer for 15 minutes at 43 rpm followed by sieving of the material through #50 sieve. The material was again blended for 15 minutes at 43 rpm. An aliquot of blended material from the top, middle and bottom of the container were taken and evaluated for TCC, VCC and strain distribution by relative abundance. The blending study results showed that the DS material was homogenously mixed with blend-sieve-blend mixing technique. The VCC/g, TCC/g and relative abundance of the three O. formigenes strains in the top, middle and bottom of the mixing container are very similar, which indicates a homogenous blend of DSs in the blending container.

A diagram of the coculture method of manufacture is provided if FIG. 14.

Manufacture of DSL. Yeast casitone fatty acids with carbohydrates (YCFAC) medium, pH 7, was prepared at 1× concentration in batches of 4 L each for Seed 1 fermentation and Seed 2 fermentation. The medium was prepared by adding the components indicated in Table 26 to 3.46 kg of water for injection, boiling for 5 to 10 minutes, then allowing the medium to cool down. Upon reaching a temperature of 50° C. or lower, the medium was sparged with N₂ while the rest of the components were added in the following order: sodium bicarbonate, 50× volatile fatty acid solution, L-cysteine HCl monohydrate, 0.5% hemin solution, and 25× vitamin solution. The pH was adjusted to 7 with 10 N NaOH or sulfuric acid, and the medium was autoclaved at 122.5° C. for 45 minutes. The medium was incubated at 37° C. for a minimum of 24 hours prior to inoculation for a contamination check.

TABLE 26
YCFAC Media

| | Quantity/4.0 L | | Final | |
|---|---|---|---|---|
| Reagent Description | of Medium | Unit | Conc. (1×) | Addition |
| Soytone | 40.00 | g | 1% w/w | Boiled for |
| D-cellobiose | 8.00 | g | 0.2% w/w | 5 to 10 minutes in |
| Yeast extract | 10.00 | g | 0.25% w/w | 3.46 kg Water for |
| Dextrose (glucose) | 20.00 | g | 0.5% w/w | Injection (WFI) |
| Maltose monohydrate | 8.00 | g | 0.2% w/w | |
| Magnesium sulfate heptahydrate | 0.36 | g | 0.009% w/w | |
| Calcium chloride dihydrate | 0.36 | g | 0.009% w/w | |
| Potassium phosphate monobasic | 1.80 | g | 0.045% w/w | |
| Potassium phosphate dibasic | 1.80 | g | 0.045% w/w | |
| Sodium chloride | 3.60 | g | 0.09% w/w | |
| Sodium bicarbonate (7.5%) | 213.0 | mL | 5.325% w/w | Added after the media |
| Volatile fatty acid solution | 11.56 | mL | 1× volatile fatty | cools down to 50° C. |
| (50×) | | | acid solution^{b} | or lower |
| L-cysteine HCI monohydrate | 4.0 | g | 0.1% w/w | |
| Hemin solution (0.5% w/w) | 8.00 | mL | 0.2% w/w | |
| Vitamin solution (25×) | 160.00 | mL | 1× vitamin | |
| | | | solution | |

A 5× concentration media was also made for use in the main fermentation. The 5× stock was made using the same proportions as described in Table 26, scaled up to 5×. The 5× media was diluted to a 1× concentration before the main fermentation process.

Resuspension medium was also made and comprised YCFAC medium with reducing agents L-cysteine HCl and riboflavin, pH 7. To prepare resuspension medium, 0.6 g of riboflavin and 2.0 g of cysteine-HCl are added per kg of YCFAC medium. The medium is stirred until completely dissolved, then titrated with 10 N NaOH or sulfuric acid to obtain a final pH of 7. The medium is filtered with a 0.22 m polyethersulfone (PES) filter. The final concentration of Riboflavin was 0.0600 and the final concentration of L-cysteine HCL was 0.2%, in YCFAC media.

The volatile fatty acid solution (50×) for the YCFAC media was made and comprised Glacial acetic acid (65.7% w/w for the 50× concentration; 1.31% w/w for the 1× concentration), Propionic acid (24.2% w/w for the 50× concentration; 0.48% w/w for the 1× concentration), Iso-butyric acid (3.1% w/w for the 50× concentration; 0.06% w/w for the 1× concentration), n-Valeric acid (3.5% w/w for the 50× concentration; 0.07% w/w for the 1× concentration), and Iso-valeric acid (3.5% w/w for the 50× concentration; 0.07% w/w for the 1× concentration).

The vitamin solution (25×) for the YCFAC media comprised Biotin powder (1.31 Quantity/6 kg WFI (g)), Folic acid (1.31 Quantity/6 kg WFI (g)), Pyridoxine hydrochloride (6.56 Quantity/6 kg WFI (g)), Thiamine-HCl-2H₂O (3.28 Quantity/6 kg WFI (g)), Riboflavin (0.13 Quantity/6 kg WFI (g)), Nicotinic acid (3.28 Quantity/6 kg WFI (g)), D-calcium pantothenate (3.28 Quantity/6 kg WFI (g)), Vitamin B12 (0.07 Quantity/6 kg WFI (g)), 4-aminobenzoic acid (3.28 Quantity/6 kg WFI (g)), and DL-alfa-lipoic acid (3.28 Quantity/6 kg WFI (g)).

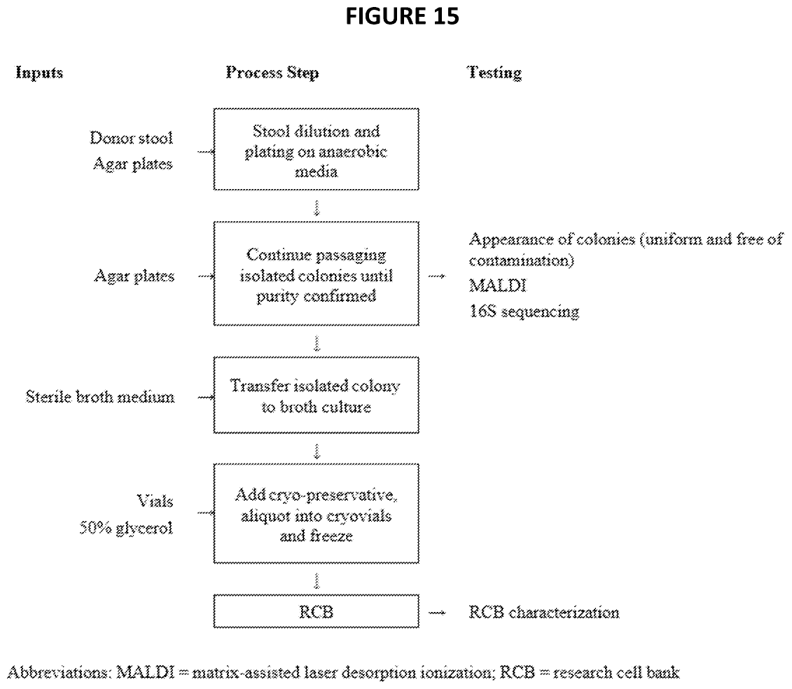

Microbial strains intended for FB-001 DS-CoC1 were isolated from stool samples obtained after extensive donor screening. An overview of the strain isolation and purification process, RCB banking, and RCB identity/purity testing is provided in FIG. 15. The entire stool sample homogenization and aliquoting was carried out under anaerobic conditions, starting with transfer of the stool sample to the anaerobic chamber within 15 to 30 minutes of the collection, followed by homogenization and addition of a 1:1 solution of PBS and 50% glycerol prior to aliquoting into 6 to 9 separate cryovials and transferring to ≤−65° C. for storage until further processing.

To isolate individual strains, fecal samples were serially diluted and then plated onto a variety of agar plates containing anaerobic microbial cultivation media (counted as passage 1). The plates were incubated at 37° C. under anaerobic conditions. Single colonies from these initial growth plates were picked for further isolation on appropriate microbial cultivation agar media plates (counted as passage 2). After incubation at 37° C., if the single-colony plating resulted in isolated colonies with uniform morphology, the culture was further characterized for strain identification. Preliminary strain identification was performed either by 16S rRNA gene sequencing or by creating and analyzing proteomic fingerprinting using high-throughput matrix-assisted laser desorption/ionization-time of flight spectrometry. If the single-colony plating resulted in multiple colony morphologies, each unique colony type was picked from this plating for further isolation on an appropriate cultivation agar plate until uniform colony morphology was achieved (counted as passage 3 or more). The passage history of each strain in FB-001 DS-CoC1 and the agar and broth medias are listed in Table 27.

TABLE 27
Isolation of Research Cell Banks Used in FB-001 DS-CoC1

| | RCB Agar Passaging | | RCB Broth Passaging | |
|---|---|---|---|---|
| FBI | | Passage | | Passage |
| Strain ID | Agar Type | # | Broth Type | # |
| FBI00001 | Bifidobacterium selective agar + 40 mM oxalate | 1 | YCFAC, pH 6.0 + 40 mM | 1 |
| | Bifidobacterium selective agar | 2 | oxalate | |
| FBI00002 | Bifidobacterium selective agar + 40 mM oxalate | 1 | YCFAC, pH 6.0 + 40 mM | 1 |
| | Bifidobacterium selective agar | 2 | oxalate | |
| FBI00010 | YCFAC-B agar | 3 | YCFAC, pH 6.0 | 1 |
| FBI00013 | YCFAC-B agar | 3 | YCFAC, pH 6.0 | 1 |
| FBI00029 | Brain heart infusion agar with hemin and | 2 | YCFAC, pH 6.0 | 1 |
| | vitamin K | | | |
| FBI00032 | Bifidobacterium selective agar | 3 | YCFAC, pH 6.0 | 1 |
| | YCFAC agar | 1 | | |
| FBI00033 | YCFAC-B agar + 20 mM oxalate | 1 | YCFAC, pH 6.0 + 20 mM | 1 |
| | YCFAC-BO 40 mM agar | 2 | oxalate | |
| | YCFAC agar | 1 | | |
| FBI00034 | Brain heart infusion agar with hemin and | 2 | YCFAC, pH 6.0 | 1 |
| | vitamin K | | | |
| FBI00043 | Reinforced clostridial agar | 2 | YCFAC, pH 6.0 | 1 |
| FBI00044 | Chocolate agar | 2 | YCFAC, pH 6.0 | 1 |
| FBI00048 | YCFAC-B agar | 3 | YCFAC, pH 6.0 | 1 |
| FBI00050 | Bifidobacterium selective agar + 40 mM oxalate | 1 | YCFAC, pH 6.0 + 40 mM | 1 |
| | YCFAC-BO 40 mM agar | 2 | oxalate | |
| FBI00051 | YCFAC-B agar | 2 | YCFAC, pH 6.0 | 1 |
| | YCFAC agar | 1 | | |
| FBI00057 | Reinforced clostridial agar | 2 | YCFAC, pH 6.0 | 1 |
| | YCFAC agar | 1 | | |
| FBI00059 | Columbia agar with 5% sheep blood | 3 | YCFAC, pH 6.0 | 1 |
| FBI00060 | YCFAC-B agar | 3 | YCFAC, pH 6.0 | 1 |
| FBI00070 | YCFAC-BO 40 mM agar | 2 | YCFAC, pH 6.0 + 40 mM | 1 |
| | YCFAC agar | 1 | oxalate | |
| FBI00071 | YCFAC-B agar | 3 | YCFAC, pH 6.0 | 1 |
| FBI00076 | YCFAC-BO 80 mM agar | 2 | YCFAC, pH 6.0 | 1 |
| | YCFAC agar | 1 | | |
| FBI00079 | Chocolate agar | 2 | YCFAC, pH 6.0 | 1 |
| FBI00087 | Brain heart infusion agar with hemin and | 2 | YCFAC, pH 6.0 | 1 |
| | vitamin K | | | |
| | YCFAC agar | 1 | | |
| FBI00093 | YCFAC-BO 40 mM agar | 2 | YCFAC, pH 6.0 | 1 |
| FBI00102 | YCFAC-BO 40 mM agar | 2 | YCFAC, pH 6.0 | 1 |
| FBI00109 | YCFAC-B agar | 4 | YCFAC, pH 6.0 | 1 |
| FBI00117 | YCFAC-BO 80 mM agar | 2 | YCFAC, pH 6.0 | 2 |
| | YCFAC agar | 1 | | |
| FBI00120 | YCFAC-BO 80 mM agar | 2 | YCFAC, pH 6.0 | 1 |
| FBI00125 | YCFAC-BO 80 mM agar | 2 | YCFAC, pH 6.0 | 1 |
| FBI00127 | YCFAC-B agar | 3 | YCFAC, pH 6.0 | 1 |
| FBI00128 | YCFAC-BO 80 mM agar | 2 | YCFAC, pH 6.0 | 1 |
| | YCFAC-BO 40 mM agar | 1 | | |
| FBI00145 | YCFAC-B agar | 3 | YCFAC, pH 6.0 | 1 |
| FBI00162 | Reinforced clostridial agar | 2 | YCFAC, pH 6.0 | 1 |
| FBI00174 | YCFAC-B agar | 2 | YCFAC, pH 6.0 | 1 |
| FBI00184 | YCFAC-B agar | 3 | YCFAC, pH 6.0 | 1 |
| FBI00190 | Brain heart infusion agar with hemin and | 2 | YCFAC, pH 6.0 | 1 |
| | vitamin K | | | |
| FBI00191 | OxyPras plus brucella blood agar | 2 | YCFAC, pH 6.0 + BBL ™ | 1 |
| | | | Vitamin K1-Hemin Solution | |
| FBI00194 | Brain heart infusion agar with hemin and | 2 | YCFAC, pH 6.0 | 1 |
| | vitamin K | | | |
| FBI00198 | YCFAC-BO 40 mM agar | 3 | YCFAC, pH 6.0 | 1 |
| FBI00199 | YCFAC-BO 40 mM agar | 3 | YCFAC, pH 6.0 | 1 |
| FBI00200 | YCFAC-B agar | 3 | YCFAC, pH 6.0 | 1 |
| FBI00201 | Columbia agar with 5% sheep blood | 3 | YCFAC, pH 6.0 | 1 |
| FBI00205 | YCFAC-BO 40 mM agar | 3 | YCFAC, pH 6.0 | 1 |
| FBI00206 | YCFAC-BO 40 mM agar | 3 | YCFAC, pH 6.0 | 1 |
| FBI00211 | YCFAC-B agar | 3 | YCFAC, pH 6.0 | 1 |
| FBI00220 | Brain heart infusion agar with hemin and | 4 | YCFAC, pH 6.0 | 1 |
| | vitamin K | | | |
| FBI00221 | Brain heart infusion agar with hemin and | 2 | YCFAC, pH 6.0 | 1 |
| | vitamin K | | | |
| FBI00236 | YCFAC-BO 40 mM agar | 2 | YCFAC pH 6.0 | 1 |
| FBI00245 | Columbia agar with 5% sheep blood | 4 | YCFAC, pH 6.0 + BBL ™ | 1 |
| | | | Vitamin K1-Hemin Solution | |
| FBI00248 | Brain heart infusion agar with hemin and | 3 | YCFAC, pH 6.0 | 1 |
| | vitamin K | | | |
| FBI00251 | Reinforced clostridial agar | 2 | YCFAC, pH 6.0 | 1 |
| FBI00254 | Brain heart infusion with hemin and vitamin K | 3 | YCFAC, pH 6.0 | 1 |
| FBI00267 | YCFAC-BO 80 mM agar | 3 | YCFAC, pH 6.0 | 1 |
| FBI00278 | Brain heart infusion agar with hemin and | 3 | YCFAC, pH 6.0 | 1 |
| | vitamin K | | | |
| FBI00288 | Brain heart infusion agar with hemin and | 3 | YCFAC, pH 6.0 | 2 |
| | vitamin K | | | |
| | YCFAC agar | 3 | | |
| | YCFAC-B agar | 1 | | |
| FBI00290 | Brain heart infusion agar with hemin and | 3 | YCFAC, pH 6.0 | 2 |
| | vitamin K | | | |

Abbreviations:
FBI = Federation Bio isolate;
RCA = reinforced clostridial agar;
RCB = research cell bank;
YCFAC = yeast casitone fatty acids with carbohydrates

To bank the RCBs used in FB-001 DS-CoCl, monocultures were inoculated into culture tubes containing appropriate broth media and incubated under anaerobic conditions at 37° C. until sufficient growth was observed. Sterile glycerol solution was added to achieve a final glycerol concentration of 25% prior to aliquoting approximately 0.2 mL into 2D-barcoded cryo-vials. After removing the cryovials from the anaerobic gas chambers, the 2D bar codes at the bottom of the vials were scanned promptly and the vials were transferred to ≤−65° C. as the final step in the banking of the RCBs.

After at least 10 hours of freezing, one vial of each purified frozen RCB was retrieved from the freezer and thawed under anaerobic conditions followed by plating on agar plates containing appropriate growth media. The plates were incubated under anaerobic conditions at 37° C. Growth on the plate was observed to confirm revival and uniform colony morphology for each purified isolate. Following confirmation of uniform colony morphology for each RCB, individual colonies were analyzed by 16S rRNA gene sequencing (see Sequence Listing). RCBs were further characterized using whole-genome sequencing followed by genome assembly. Strain-level identification was performed using both 16S rRNA gene sequences and whole-genome assemblies.

An explicit criterion for inclusion of each strain in FB-001 DS-CoC1 was demonstrated susceptibility to at least 2 FDA-approved antibiotics. The anaerobic microbes in the FB-001 DS-CoC1 were tested against multiple FDA-approved, clinically relevant antimicrobials, most of which show especially potent activity against anaerobes. All strains in FB-001 DS-CoC1 were found to demonstrate sensitivity in vitro to 2 or more clinically relevant antibiotics, implying a straightforward means for biological control. Importantly, no strain in the FB-001 DS-CoC1 were resistant to both clindamycin and amoxicillin-clavulanate, suggesting that a combination of the 2 agents could cover all FB-001 DS-CoC1 strains.

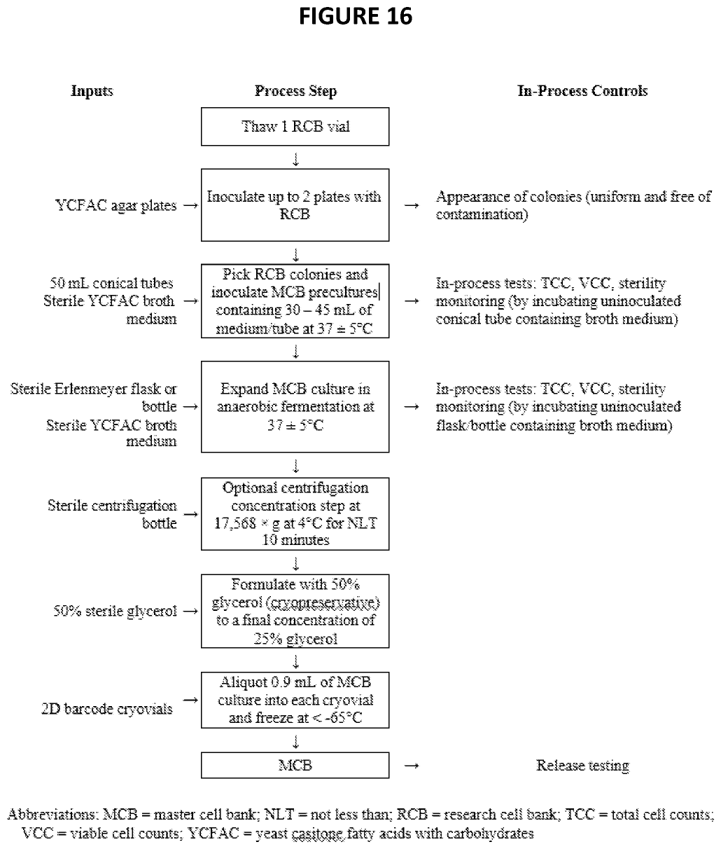

Fgu described in FIG. 16. The first step of MCB generation for DS-CoC1 strains involved reviving each RCB by plating on YCFAC agar plates followed by incubation under anaerobic conditions at 37° C. Isolated colonies were used for inoculating MCB precultures in 30 to 45 mL of YCFAC broth and were incubated anaerobically at 37° C. Each MCB was passaged 2 to 3 times in YCFAC broth prior to banking. Growth of precultures was monitored using total cell counts and viable cell counts to determine suitable time, inoculation, and culture volumes for MCB cultures. Sterility monitoring was performed by incubating a sterile agar plate or broth during the entire culturing process. A minimum total cell count of 2×10⁸ cells per mL was targeted for the harvest of the MCB culture. When required, cells were harvested by centrifugation to allow concentration of the biomass. Sterile glycerol was added as cryoprotectant to a final concentration of 25% v/v prior to aliquoting cells from MCB culture into 2D barcoded cryovials. The barcodes of cryovials were scanned and entered into an electronic inventory system, then the vials are transferred to long-term storage at ≤−65° C. All MCBs are stored in at least 2 physically distinct locations.

Manufacture of DS2. The same YCFAC media used for DS1 was used for DS2. Similarly, the same general strain isolation methods were used as described above for DS1. The specific agar types, passages, and broth types used for DS2 strains is provided in Table 28.

TABLE 28
Isolation of Research Cell Banks Used in FB-001 DS-CoC2

| FBI | RCB Agar Passaging | | RCB Broth Passaging | |
|---|---|---|---|---|
| Strain ID | Agar Type | Passage # | Broth Type | Passage # |
| FBI00004 | YCFAC-B agar + 80 mM oxalate | 1 | YCFAC, pH 6.0 + 80 mM | 1 |
| | YCFAC-BO 80 mM agar | 2 | oxalate | |
| FBI00012 | Bacteroides bile esculin agar | 2 | YCFAC, pH 6.0 | 1 |
| | YCFAC-B agar | 1 | | |
| FBI00015 | YCFAC-B agar | 2 | YCFAC, pH 6.0 | 1 |
| | YCFAC agar | 1 | | |
| FBI00018 | Bifidobacterium selective agar | 3 | YCFAC, pH 6.0 | 1 |
| FBI00019 | Bacteroides bile esculin agar | 2 | YCFAC, pH 6.0 | 1 |
| | YCFAC-B agar | 1 | | |
| FBI00021 | YCFAC-B agar + 80 mM oxalate | 1 | YCFAC, pH 6.0 + 80 mM | 1 |
| | YCFAC-BO 80 mM agar | 2 | oxalate | |
| FBI00038 | Chocolate agar | 2 | YCFAC, pH 6.0 | 1 |
| FBI00040 | Bacteroides bile esculin agar | 2 | YCFAC, pH 6.0 | 1 |
| | YCFAC-B agar | 1 | | |
| FBI00046 | YCFAC-B agar | 3 | YCFAC, pH 6.0 | 1 |
| FBI00061 | Bacteroides bile esculin | 2 | YCFAC, pH 6.0 | 1 |
| | Brain heart infusion agar with hemin and | 1 | | |
| | vitamin K | | | |
| FBI00066 | Bacteroides bile esculin | 2 | YCFAC, pH 6.0 | 1 |
| | YCFAC-B agar | 1 | | |
| FBI00075 | YCFAC-B agar | 3 | YCFAC, pH 6.0 | 1 |
| FBI00077 | Lactobacillus MRS agar + 20 mM | 2 | YCFAC, pH 6.0 | 1 |
| | oxalate | | | |
| | YCFAC | 1 | | |
| FBI00080 | Lactobacillus MRS agar + 40 mM | 2 | YCFAC, pH 6.0 | 1 |
| | oxalate | | | |
| | YCFAC agar | 1 | | |
| FBI00081 | Columbia agar with 5% sheep blood | 3 | YCFAC, pH 6.0 | 1 |
| FBI00085 | Reinforced clostridial agar | 2 | YCFAC, pH 6.0 | 1 |
| FBI00092 | YCFAC-BO 40 mM agar | 2 | YCFAC, pH 6.0 | 1 |
| FBI00097 | Brain heart infusion agar with hemin and | 2 | YCFAC, pH 6.0 | 2 |
| | vitamin K | | | |
| | YCFAC agar | 2 | | |
| FBI00099 | Chocolate agar | 2 | YCFAC, pH 6.0 | 1 |
| FBI00112 | Brain heart infusion agar with hemin and | 2 | YCFAC, pH 6.0 | 1 |
| | vitamin K | | | |
| | YCFAC agar | 1 | | |
| FBI00132 | YCFAC-BO 80 mM agar | 2 | YCFAC, pH 6.0 | 1 |
| | YCFAC-BO 80 mM agar | 1 | | |
| FBI00137 | YCFAC-BO 40 mM agar | 2 | YCFAC, pH 6.0 | 1 |
| FBI00140 | YCFAC-BO 160 mM agar | 1 | YCFAC, pH 6.0 + 80 mM | 1 |
| | YCFAC-BO 80 mM agar | 2 | oxalate | |
| FBI00149 | YCFAC-BO 80 mM agar | 1 | YCFAC, pH 6.0 | 1 |
| | YCFAC-BO 80 mM agar | 1 | | |
| | YCFAC-BO 40 mM agar | 1 | | |
| FBI00151 | YCFAC-BO 80 mM agar | 1 | YCFAC, pH 6.0 | 1 |
| | YCFAC-BO 80 mM agar | 1 | | |
| | YCFAC-BO 40 mM agar | 2 | | |
| FBI00176 | Brain heart infusion agar with hemin and | 2 | YCFAC, pH 6.0 | 1 |
| | vitamin K | | | |
| FBI00189 | YCFAC-BO 40 mM agar | 3 | YCFAC, pH 6.0 | 1 |
| | YCFAC-BO 40 mM agar | 1 | | |
| FBI00197 | YCFAC-B agar | 3 | YCFAC, pH 6.0 | 1 |
| FBI00208 | YCFAC-BO 40 mM agar | 3 | YCFAC, pH 6.0 | 1 |
| FBI00212 | YCFAC-B agar | 3 | YCFAC, pH 6.0 | 1 |
| FBI00224 | YCFAC-BO 40 mM agar | 3 | YCFAC, pH 6.0 | 1 |
| FBI00226 | YCFAC-BO 40 mM agar | 3 | YCFAC, pH 6.0 with BBL ™ | 1 |
| | | | Vitamin K1-Hemin Solution | |
| FBI00229 | Brain heart infusion agar with hemin and | 4 | Thioglycollate broth with | 1 |
| | vitamin K | | hemin and vitamin K | |
| FBI00233 | Brain heart infusion agar with hemin and | 4 | YCFAC, pH 6.0 with BBL ™ | 1 |
| | vitamin K | | Vitamin K1-Hemin Solution | |
| FBI00235 | Brain heart infusion agar with hemin and | 4 | YCFAC, pH 6.0 | 1 |
| | vitamin K | | | |
| FBI00237 | Brain heart infusion agar with hemin and | 3 | YCFAC, pH 6.0 | 1 |
| | vitamin K | | | |
| FBI00243 | Brain heart infusion agar with hemin and | 4 | YCFAC, pH 6.0 | 1 |
| | vitamin K | | | |
| | YCFAC agar | 1 | | |
| FBI00244 | YCFAC-BO 40 mM agar | 3 | YCFAC, pH 6.0 | 1 |
| | YCFAC-B agar + 40 mM oxalate | 3 | | |
| | YCFAC agar | 1 | | |
| FBI00258 | Modified Eggerth-Gagnon medium agara | 3 | Thioglycollate broth with | 1 |
| | | | hemin and vitamin K | |
| FBI00260 | Brain heart infusion agar with hemin and | 3 | YCFAC, pH 6.0 | 1 |
| | vitamin K | | | |
| FBI00263 | Modified Eggerth-Gagnon medium agara | 3 | YCFAC, pH 6.0 | 1 |
| FBI00270 | Columbia agar with 5% sheep blood + | 2 | SAB mediac | 1 |
| | antibioticsb | | | |
| | Columbia agar with 5% sheep blood | 3 | | |
| FBI00273 | Brain heart infusion agar with hemin and | 3 | YCFAC, pH 6.0 | 1 |
| | vitamin K | | | |
| FBI00277 | Brain heart infusion agar with hemin and | 3 | YCFAC, pH 6.0 | 1 |
| | vitamin K | | | |
| FBI00292 | Columbia agar with 5% sheep blood | 3 | SAB mediac | 1 |

Abbreviations:
FBI = Federation Bio isolate;
RCB = research cell bank;
YCFAC = yeast casitone fatty acids with carbohydrates,
YCFAC-B = yeast casitone fatty acids with carbohydrates and sheep blood;
YCFAC-BO = yeast casitone fatty acids with carbohydrates, sheep blood, and oxalate
^{a} Modified Eggerth-Gagnon medium agar is prepared in house and consists of peptone (1% w/v), Na₂HPO₄ (0.32% w/v), mucin (0.2% w/v), BactoAgar (1.5% w/v), and sheep blood (5% v/v), pH 7.45.
^{b} Antibiotics used for isolation of FBI00270 included vancomycin (100 μg/mL), penicillin 100 units/mL, streptomycin (100 μg/mL), and amphotericin B (0.25 μg /mL)
^{c} SAB media was prepared at described in (Khelaifia, Raoult etal. 2013).

Characterization and banking of the DS2 strains were performed as described above for DS1. It is important to note that while not all DS2 strains were sensitive to both clindamycin and amoxicillin-clavulanate as were the D S1 strains, all strains were still sensitive to at least 2 FDA approved antibiotics.

Manufacture of DS3. The same YCFAC media used for DS1 was used for DS2. Similarly, the same general strain isolation methods were used as described above for DS1. The specific agar types, passages, and broth types used for DS2 strains is provided in Table 29.

TABLE 29
Isolation of Research Cell Banks Used in FB-001 DS-CoC3

| | RCB Agar Passaging | | RCB Broth Passaging | |
|---|---|---|---|---|
| FBI | | Passage | | Passage |
| Strain ID | Agar Type | # | Broth Type | # |
| FBI00009 | YCFAC-B agar | 3 | YCFAC, pH 6.0 | 1 |
| FBI00011 | Bifidobacterium selective agar | 3 | YCFAC, pH 6.0 | 1 |
| FBI00016 | YCFAC-B agar | 3 | YCFAC, pH 6.0 | 1 |
| FBI00020 | Bifidobacterium selective | 1 | YCFAC, pH 6.0 with 40 mM | 1 |
| | agar + 40 mM oxalate | | Oxalate | |
| | YCFAC-BO 40 mM agar | 1 | | |
| | YCFAC-BO 40 mM agar | 1 | | |
| FBI00025 | Chocolate agar | 2 | YCFAC, pH 6.0 | 1 |
| FBI00027 | Brain-heart infusion agar with | 2 | YCFAC, pH 6.0 | 1 |
| | hemin and vitamin K | | | |
| FBI00030 | YCFAC-B agar | 3 | YCFAC, pH 6.0 | 1 |
| FBI00047 | YCFAC-B agar | 3 | YCFAC, pH 6.0 | 1 |
| FBI00052 | YCFAC-BO 40 mM agar | 2 | YCFAC, pH 6.0 with 40 mM | 1 |
| | YCFAC agar | 1 | Oxalate | |
| FBI00053 | YCFAC-BO 40 mM agar | 2 | YCFAC, pH 6.0 with 40 mM | 1 |
| | YCFAC agar | 1 | Oxalate | |
| FBI00056 | YCFAC-BO 80 mM agar | 2 | YCFAC, pH 6.0 with 80 mM | 1 |
| | YCFAC agar | 1 | Oxalate | |
| FBI00062 | YCFAC-B agar | 3 | YCFAC, pH 6.0 | 1 |
| FBI00078 | YCFAC-B agar | 2 | YCFAC, pH 6.0 | 1 |
| FBI00096 | Brain-heart infusion agar with | 2 | YCFAC, pH 6.0 | 1 |
| | hemin and vitamin K | | | |
| | YCFAC agar | 1 | | |
| FBI00104 | Brain-heart infusion agar with | 2 | YCFAC, pH 6.0 | 1 |
| | hemin and vitamin K | | | |
| | YCFAC agar | 1 | | |
| FBI00110 | YCFAC-BO 80 mM agar | 2 | YCFAC, pH 6.0 | 1 |
| | YCFAC agar | 1 | | |
| | YCFAC-B agar | 1 | | |
| FBI00111 | YCFAC-BO 80 mM agar | 2 | YCFAC, pH 6.0 | 1 |
| | YCFAC agar | 1 | | |
| | YCFAC-B agar | 1 | | |
| FBI00113 | Brain-heart infusion agar with | 2 | YCFAC, pH 6.0 | 1 |
| | hemin and vitamin K | | | |
| | YCFAC-B agar | 1 | | |
| FBI00115 | Brain-heart infusion agar with | 2 | YCFAC, pH 6.0 | 2 |
| | hemin and vitamin K | | | |
| | YCFAC agar | 3 | | |
| FBI00116 | Bifidobacterium selective | 1 | YCFAC, pH 6.0 with 40 mM | 1 |
| | agar + 40 mM oxalate | | Oxalate | |
| | Bifidobacterium selective agar | 2 | YCFAC, pH 6.0 | 1 |
| FBI00123 | YCFAC-BO 160 mM agar | 1 | YCFAC, pH 6.0 | 1 |
| | YCFAC-B agar | 2 | | |
| FBI00124 | YCFAC-B agar | 3 | YCFAC, pH 6.0 | 1 |
| FBI00126 | YCFAC-BO 40 mM agar | 1 | YCFAC, pH 6.0 | 1 |
| | YCFAC-BO 40 mM agar | 1 | | |
| FBI00135 | YCFAC-B agar | 3 | YCFAC, pH 6.0 | 1 |
| FBI00147 | YCFAC-BO 80 mM agar | 1 | YCFAC, pH 6.0 | 1 |
| | YCFAC-BO 80 mM agar | 1 | | |
| | YCFAC-BO 40 mM agar | 1 | | |
| FBI00159 | YCFAC-BO 160 mM agar | 1 | YCFAC, pH 6.0 | 1 |
| | YCFAC-BO 80 mM agar | 2 | | |
| FBI00167 | YCFAC-B agar | 2 | YCFAC, pH 6.0 | 1 |
| FBI00170 | YCFAC-BO 80 mM agar | 1 | YCFAC, pH 6.0 | 2 |
| | YCFAC-BO 80 mM agar | 1 | | |
| | YCFAC-BO 40 mM agar | 1 | | |
| FBI00232 | Columbia agar with 5% sheep | 4 | YCFAC, pH 6.0 | 1 |
| | blood | | | |
| FBI00255 | YCFAC-BO 80 mM agar | 3 | YCFAC, pH 6.0 | 1 |
| FBI00271 | Brain-heart infusion agar with | 3 | YCFAC, pH 6.0 | 1 |
| | hemin and vitamin K | | | |

Abbreviations:
FBI = Federation Bio isolate;
RCB = research cell bank;
YCFAC = yeast casitone fatty acids with carbohydrates,
YCFAC-B = yeast casitone fatty acids with carbohydrates and sheep blood;
YCFAC-BO = yeast casitone fatty acids with carbohydrates, sheep blood and oxalate

Characterization and banking of the DS3 strains were performed as described above for DS1. It is important to note that while not all DS2 strains were sensitive to both clindamycin and amoxicillin-clavulanate as were the D S1 strains, all strains were still sensitive to at least 2 FDA approved antibiotics.

Manufacture of DS4. YCFAC media with ammonium sulfate, pH 7 for Seed 1 Fermentation was prepared at 1× concentration in batches of 4 L. The medium is prepared by adding the components indicated in Table 30 to 3.46 kg of water for injection and boiling for 5 to 10 minutes. Then the media was sparged for at least 30 minutes with N₂ and allowed to cool down. Upon reaching a temperature of 50° C. or lower, the rest of the components were added in the following order while sparging continues: sodium bicarbonate, 50× volatile fatty acid solution, L-cysteine HCl monohydrate, and 0.5% hemin solution. The medium was adjusted to a pH of 7 with 10 N NaOH or sulfuric acid and was autoclaved at 122.5° C. for 45 minutes. Vitamin solution (25×) was filtered using a 0.22 m filter and added post-sterilization. The medium was incubated at 37° C. for a minimum of 24 hours prior to inoculation for a contamination check.

TABLE 30
Yeast Casitone Fatty Acids With Carbohydrates Medium Composition (1×) For Seed 1 Fermentation

| | Quantity/4.0 L | | Final | |
|---|---|---|---|---|
| Reagent Description | of Medium | Unit | Conc. (1×) | Addition |
| Soytone | 40.00 | g | 1% (w/w) | Boiled for |
| D-cellobiose | 8.00 | g | 0.2% (w/w) | 5 to 10 minutes in |
| Yeast extract | 10.00 | g | 0.25% (w/w) | 3.46 kg WFI; |
| Dextrose (glucose) | 20.00 | g | 0.5% (w/w) | sparging initiated |
| Maltose monohydrate | 8.00 | g | 0.2% (w/w) | |
| Magnesium sulfate heptahydrate | 0.36 | g | 0.009% (w/w) | |
| Calcium chloride dihydrate | 0.36 | g | 0.009% (w/w) | |
| Potassium phosphate monobasic | 1.80 | g | 0.045% (w/w) | |
| Potassium phosphate dibasic | 1.80 | g | 0.045% (w/w) | |
| Sodium chloride | 3.60 | g | 0.09% (w/w) | |
| Ammonium sulfate | 3.60 | g | 0.09% (w/w) | |
| Sodium bicarbonate (7.5% w/w) | 213 | mL | 5.325% (w/w) | Added after the media |
| Volatile fatty acid solution | 11.6 | mL | 1× volatile fatty | cools down to 50° C. |
| (50×) | | | acid solution | or lower (sparging |
| L-cysteine HCl monohydrate | 4.0 | g | 0.1% | continues) |
| Hemin solution (0.5% w/w) | 8.00 | mL | 0.2% w/w hemin | |
| Vitamin solution (25×) | 160.00 | mL | 1× vitamin | Added post- |
| | | | solution | sterilization |

YCFAC medium with ammonium sulfate, threonine, and N-acetylgalactosamine, pH 7.4 for Seed 2 Fermentation (Stage 1 and Stage 2) is prepared at 1× concentration in batches of 4 L. The medium is prepared by adding the components indicated in Table 31 to 3.46 kg of water for injection and boiling for 5 to 10 minutes. Then the media is sparged for at least 30 minutes with N₂ and allowed to cool down. Upon reaching a temperature of 50° C. or lower, the rest of the components are added in the following order while sparging continues: sodium bicarbonate, 50× volatile fatty acid solution, L-cysteine HCl monohydrate, and 0.5% hemin solution. The medium is adjusted to a pH of 7 with 10 NNaOH or sulfuric acid and is autoclaved at 122.5° C. for 45 minutes. Sterile 25× vitamin solution (25×), threonine solution, and N-acetylgalactosamine solution are added post-sterilization. The medium is incubated at 37° C. for a minimum of 24 hours prior to inoculation for a contamination check.

TABLE 31
Yeast Casitone Fatty Acids With Carbohydrates Medium Composition (1×) For Seed 2 Fermentation

| | Quantity/4.0 L | | Final | |
|---|---|---|---|---|
| Reagent Description | of Medium | Unit | Conc. (1×) | Addition |
| Soytone | 40.00 | g | 1% (w/w) | Boiled for |
| D-cellobiose | 8.00 | g | 0.2% (w/w) | 5 to 10 minutes in |
| Yeast extract | 10.00 | g | 0.25% (w/w) | 3.46 kg WFI; sparging |
| Dextrose (glucose) | 20.00 | g | 0.5% (w/w) | initiated |
| Maltose monohydrate | 8.00 | g | 0.2% (w/w) | |
| Magnesium sulfate heptahydrate | 0.36 | g | 0.009% (w/w) | |
| Calcium chloride dihydrate | 0.36 | g | 0.009% (w/w) | |
| Potassium phosphate monobasic | 1.80 | g | 0.045% (w/w) | |
| Potassium phosphate dibasic | 1.80 | g | 0.045% (w/w) | |
| Sodium chloride | 3.60 | g | 0.09% (w/w) | |
| Ammonium sulfate | 3.60 | g | 0.09% (w/w) | |
| Sodium bicarbonate (7.5% w/w) | 213 | mL | 5.325% (w/w) | Added after the |
| Volatile fatty acid solution (50×) | 11.6 | mL | 1× volatile fatty | media cools down |
| | | | acid solution | to 50° C. or lower |
| L-cysteine HCl monohydrate | 4.0 | g | 0.1% | (sparging continues) |
| Hemin solution (0.5% w/w) | 8.00 | mL | 0.2% w/w hemin | |
| Vitamin solution (25×) | 160.00 | mL | 1× vitamin | Added post- |
| | | | solution | sterilization |
| N-acetylgalactosamine solution | 20 | mL | 0.5% (w/w) | |
| (1% w/w) | | | | |
| Threonine | 59.6 | g | 1.5% (w/w) | |

YCFAC medium with ammonium sulfate and threonine, pH 7, used for the main fermentation, is prepared at 5×. The 5× medium is prepared by adding the components indicated in Table 32 to 40.0 kg of water for injection, mixing, then autoclaving. The medium is incubated at 37° C. for a minimum of 24 hours prior to inoculation for a contamination check. After pumping the 5× solution into the fermenter, 50× volatile fatty acid solution, threonine solution, 25× vitamin solution, L-cysteine HCl solution, and WFI are added for a final 1× concentration.

TABLE 32
Yeast Casitone Fatty Acids with Carbohydrates Medium Composition (5×)

| | Quantity/60.0 kg | | |
|---|---|---|---|
| | YCFAC (5×) | | Final Conc. |
| Reagent Description | Medium | Unit | (1×) |
| Soytone | 3.00 | kg | 1% (w/w) |
| D-(+) cellobiose | 600 | g | 0.2% (w/w) |
| Yeast extract | 750 | g | 0.25% (w/w) |
| Dextrose (glucose) | 1.50 | kg | 0.5% (w/w) |
| Maltose monohydrate | 600.0 | g | 0.2% (w/w) |
| Magnesium sulfate heptahydrate | 27.0 | g | 0.009% (w/w) |
| Calcium chloride dihydrate | 27.0 | g | 0.009% (w/w) |
| Potassium phosphate monobasic | 135 | g | 0.045% (w/w) |
| Potassium phosphate dibasic | 135 | g | 0.045% (w/w) |
| Sodium chloride | 270 | g | 0.09% (w/w) |
| Ammonium sulfate | 270 | g | 0.09% (w/w) |
| Sodium bicarbonate (7.5% w/w) | 16.0 | kg | 5.33% (w/w) |
| Hemin solution (0.5% w/w) | 600 | mL | 0.2% (w/w) |

| | |
|---|---|
| Volatile fatty acid solution (50×) | 1× |
| L-cysteine HCl monohydrate solution (3% w/w) | 3.32% (w/w) |
| Vitamin solution (25×) | 1× |
| Threonine solution (7.2% w/w) | 4.0% (w/w) |

The specific agar types, passages, and broth types used for DS2 strains is provided in Table 33.

TABLE 33
Isolation of Research Cell Banks Used in FB-001 DS-CoC4

| FBI Strain | RCB Agar Passaging | | RCB Broth Passaging | |
|---|---|---|---|---|
| ID | Agar Type | Passage # | Broth Type | Passage # |
| FBI00022 | Bacteroides bile esculin agar | 2 | YCFAC, pH 6.0 | 1 |
| | YCFAC-B agar | 1 | | |
| FBI00049 | YCFAC-B agar | 3 | YCFAC, pH 6.0 | 1 |
| FBI00068 | Bicarbonate-buffered basal | 2 | YCFAC, pH 6.0 | 2 |
| | medium agar^{a} | | | |
| | YCFAC-B agar | 1 | | |
| FBI00069 | Bifidobacterium selective agar + | 1 | YCFAC, pH 6.0 | 1 |
| | 40 mM oxalate | | | |
| | Bifidobacterium selective agar | 2 | | |
| FBI00152 | YCFAC-B agar | 2 | YCFAC, pH 6.0 with | 1 |
| | | | BBL ™ Vitamin K1- | |
| | | | Hemin Solution | |
| FBI00165 | Brain-heart infusion agar with | 3 | YCFAC, pH 6.0 | 1 |
| | hemin and vitamin K | | | |
| FBI00171 | YCFAC-BO 80 mM agar | 1 | YCFAC, pH 6.0 | 2 |
| | YCFAC-BO 80 mM agar | 1 | | |
| | YCFAC-BO 40 mM agar | 1 | | |
| FBI00175 | YCFAC-B agar | 2 | YCFAC, pH 6.0 with BBL | 1 |
| | | | Vitamin K1-Hemin Solution | |
| FBI00177 | Bacteroides bile esculin agar | 2 | YCFAC, pH 6.0 | 1 |
| FBI00180 | Bacteroides bile esculin agar | 2 | YCFAC, pH 6.0 | 1 |
| FBI00182 | YCFAC-B agar | 2 | YCFAC, pH 6.0 | 1 |
| FBI00238 | Columbia agar with 5% sheep | 3 | YCFAC, pH 6.0 | 1 |
| | blood | | | |
| FBI00269 | Brain-heart infusion agar with | 3 | YCFAC, pH 6.0 | 1 |
| | hemin and vitamin K | | | |
| FBI00274 | YCFAC-BO 80 mM agar | 3 | YCFAC, pH 6.0 | 1 |
| FBI00281 | Reinforced clostridial agar | 3 | YCFAC, pH 6.0 | 1 |

Abbreviations:
FBI = Federation Bio isoate;
RCA = reinforced clostridial agar;
RCB = research cell bank;
YCFAC = yeast casitone fatty acids with carbohydrates,
YCFAC-B = yeast casitone fatty acids with carbohydrates and sheep blood;
YCFAC-BO = yeast casitone fatty acids with carbohydrates, sheep blood, and oxalate; Bicarbonate-buffered basal medium was prepared as described in Derrien 2004 (Derrien, Vaughan et al. 2004).

Characterization and banking of the DS4 strains was performed as described above for DS1. It is important to note that while not all DS2 strains were sensitive to both clindamycin and amoxicillin-clavulanate as were the DS1 strains, all strains were still sensitive to at least 2 FDA approved antibiotics.

### Example 7: Functional Characterization of DS1-7

FB-001 was characterized through 16S sequence identity, macronutrient utilization, metabolite production and Biolog analysis of individual strains. At the species level, FB-001 was characterized by the DNA sequences of 16S rRNA genes which represent 100 species. 16S sequence length varied by strain, from a minimum of 1177 bp (FBI00109, Coprococcus comes) to a maximum of 1532 bp (FBI00087, Clostridium scindens). The 148 16S DNA sequences uniquely identified the majority of the 148 strains within FB-001, with exceptions for closely related strains such as two of the Oxalobacter formigenes strains (FBI00133 and FBI00289) which share identical 16S sequences. To provide phenotypic characterization, Biolog assays were used to characterize the strains in FB-001, as described below.

Biolog phenotype assays were used to determine unique macronutrient signatures for FB-001 strains. These data provide empirical characterization of growth features of each strain. The 148 strains of FB-001 fit into several broad categories of growth characteristics based on our Biolog analyses: 98 strains showed positive growth signatures; 41 strains did not have positive growth signatures; 9 were not tested using Biolog due to insufficient growth. Table 34 shows the 98 strains with positive growth signatures, with the specific macronutrients that supported growth listed along with the Genus species identification of each strain. Of the 98 strains with positive growth signatures, 60 were tested against the 190 individual carbon and energy sources present in the 96 well plate format of PM1 and 2 plates and the remaining 38 were tested using 2 plates alone. Each 96 well plate contains one negative control well that lacks any additional carbon or energy source. The total number of substrates utilized by any single strain in this assay showed great diversity, ranging from 1 to 59 substrates that yield growth. Furthermore, each of the 98 strains with growth on at least one substrate presented with an entirely unique growth fingerprint, or combination of permissive growth substrates, relative to every other strain in the set.

TABLE 34
Characterization of strain-level macronutrient utilization by Biolog assay in 98
strains with positive growth signatures. For each strain, the Biolog PM plates tested are given
along with the Genus species identity and the macronutrients that supported growth. Positive
growth is defined as an increase of 0.1 or more in optical density at 600 nm above the negative control
that container no supplied carbon and energy source.

| Strain | PM | Macronutrient growth |
|---|---|---|
| FBI00001 | 1, 2 | 2-Deoxy-D-Ribose; D-Fructose; D-Fructose-6-Phosphate; D-Galactose; D- |
| | | Galacturonic Acid; D-Gluconic Acid; D-Glucosamine; D-Glucuronic Acid; D- |
| | | Mannose; D-Ribose; D-Saccharic Acid; D-Trehalose; D-Xylose; Inosine; L- |
| | | Arabinose; L-Galactonic Acid-g-Lactone; Maltose; Mucic Acid; N-Acetyl-D- |
| | | Glucosamine; N-Acetyl-Neuraminic Acid; Sucrose; Thymidine; Uridine; a-D- |
| | | Glucose; b-D-Allose |
| FBI00002 | 1, 2 | D-Galactose; a-D-Glucose; a-D-Lactose |
| FBI00004 | 1, 2 | D-Glucose-6-Phosphate; Maltose; Maltotriose |
| FBI00010 | 1, 2 | D-Arabinose; D-Cellobiose; D-Fructose; D-Fucose; D-Galactose; D-Mannose; |
| | | D-Raffinose; D-Ribose; D-Sorbitol; D-Xylose; L-Arabinose; L-Fucose; L- |
| | | Rhamnose; Maltose; Maltotriose; Stachyose; a-D-Glucose; a-D-Lactose |
| FBI00013 | 1, 2 | Caproic Acid; D-Melibiose; L-Pyroglutamic Acid; Melibionic Acid; N-Acetyl- |
| | | D-Glucosamine; N-Acetyl-L-Glutamic Acid; Oxalic Acid; a-D-Glucose; a-D- |
| | | Lactose |
| FBI00015 | 1, 2 | 3-Hydroxy 2-Butanone; 3-Methyl Glucose; Amygdalin; Arbutin; D- |
| | | Cellobiose; D-Glucosamine; D-Glucuronic Acid; D-Melibiose; D-Raffinose; |
| | | Inulin; L-Galactonic Acid-g-Lactone; Lactulose; N-Acetyl-D-Galactosamine; |
| | | N-Acetyl-b-D-Mannosamine; Palatinose; Salicin; a-D-Lactose; a-Methyl-D- |
| | | Galactoside; b-Cyclodextrin; b-Methyl-D-Galactoside |
| FBI00025 | 1, 2 | D-Fructose; D-Fucose; D-Galactose; D-Melibiose; D-Raffinose; D-Xylose; |
| | | Glycogen; L-Arabinose; Lactulose; Maltose; Maltotriose; Stachyose; Sucrose; |
| | | a-D-Glucose; a-D-Lactose |
| FBI00027 | 1, 2 | D-Cellobiose; D-Galactose; D-Glucosamine; D-Raffinose; L-Tartaric Acid; |
| | | Lactulose; Maltitol; Maltose; Maltotriose; Palatinose; Pectin; Stachyose; |
| | | Sucrose; a-D-Glucose; a-D-Lactose; a-Methyl-D-Galactoside; a-Methyl-D- |
| | | Glucoside; b-Methyl-D-Galactoside; b-Methyl-D-Glucoside; b-Methyl-D- |
| | | Xyloside |
| FBI00030 | 1, 2 | D-Mannitol; D-Trehalose; D-Xylose; Glycogen; Palatinose; a-D-Lactose |
| FBI00033 | 1, 2 | Lactulose; a-D-Lactose |
| FBI00044 | 1, 2 | Amygdalin; Arbutin; D-Arabinose; D-Cellobiose; D-Fructose; D-Galactose; |
| | | D-Glucosamine; D-Mannitol; D-Melezitose; D-Melibiose; D-Raffinose; D- |
| | | Sorbitol; D-Trehalose; D-Xylose; Gentiobiose; L-Arabinose; L-Fucose; |
| | | Lactitol; Lactulose; Maltitol; Maltose; Maltotriose; N-Acetyl-Neuraminic |
| | | Acid; Palatinose; Pectin; Salicin; Stachyose; Sucrose; Turanose; a-D-Glucose; |
| | | a-D-Lactose; b-Methyl-D-Galactoside; b-Methyl-D-Glucoside |
| FBI00046 | 1, 2 | D-Arabinose; D-Cellobiose; D-Fructose; D-Galactose; D-Galacturonic Acid; |
| | | D-Glucosamine; D-Glucuronic Acid; D-Mannose; D-Melezitose; D-Melibiose; |
| | | D-Raffinose; D-Ribose; D-Trehalose; D-Xylose; Gentiobiose; L-Arabinose; L- |
| | | Fucose; L-Galactonic Acid-g-Lactone; Lactitol; Lactulose; Maltitol; Maltose; |
| | | Maltotriose; N-Acetyl-D-Galactosamine; N-Acetyl-D-Glucosamine; |
| | | Palatinose; Pectin; Salicin; Stachyose; Sucrose; Turanose; Uridine; a-D- |
| | | Glucose; a-D-Lactose; a-Methyl-D-Glucoside; b-Methyl-D-Galactoside |
| FBI00047 | 1, 2 | D-Gluconic Acid |
| FBI00048 | 1, 2 | Arbutin; D-Cellobiose; D-Fructose; D-Galactose; D-Glucosamine; D- |
| | | Glucosaminic Acid; D-Melibiose; D-Raffinose; Gentiobiose; L-Arabinose; |
| | | Lactitol; Lactulose; Maltitol; Maltose; Maltotriose; Melibionic Acid; |
| | | Palatinose; Pectin; Salicin; Stachyose; Sucrose; Turanose; a-D-Glucose; a-D- |
| | | Lactose; a-Methyl-D-Glucoside; b-Methyl-D-Galactoside; b-Methyl-D- |
| | | Glucoside |
| FBI00050 | 1, 2 | 3-Methyl Glucose; Amygdalin; Chondroitin Sulfate C; D-Cellobiose; D- |
| | | Fructose; D-Galactose; D-Galacturonic Acid; D-Glucosamine; D-Glucuronic |
| | | Acid; D-Mannose; D-Melibiose; D-Raffinose; D-Xylose; Dextrin; |
| | | Gentiobiose; Glycogen; Inulin; L-Fucose; L-Galactonic Acid-g-Lactone; L- |
| | | Lyxose; L-Rhamnose; Lactulose; Laminarin; Maltose; Maltotriose; N-Acetyl- |
| | | D-Galactosamine; N-Acetyl-D-Glucosamine; N-Acetyl-b-D-Mannosamine; |
| | | Palatinose; Pectin; Stachyose; Sucrose; Thymidine; Uridine; a-Cyclodextrin; |
| | | a-D-Glucose; a-D-Lactose; b-Cyclodextrin; b-Methyl-D-Galactoside; g- |
| | | Cyclodextrin |
| FBI00051 | 1, 2 | D-Fructose; D-Galactose; D-Galacturonic Acid; D-Sorbitol; Inulin; L- |
| | | Arabinose; L-Galactonic Acid-g-Lactone; L-Tartaric Acid; Maltose; |
| | | Maltotriose; N-Acetyl-Neuraminic Acid; a-D-Glucose |
| FBI00053 | 1, 2 | D-Galacturonic Acid; D-Gluconic Acid; D-Glucuronic Acid; L-Galactonic |
| | | Acid-g-Lactone |
| FBI00057 | 1, 2 | Arbutin; D-Cellobiose; D-Fructose; D-Glucosamine; D-Raffinose; D-Sorbitol; |
| | | L-Arabinose; Maltose; Maltotriose; N-Acetyl-D-Galactosamine; N-Acetyl- |
| | | Neuraminic Acid; Stachyose; Turanose; a-D-Glucose; a-D-Lactose; m-Inositol |
| FBI00059 | 1, 2 | Amygdalin; Arbutin; D-Arabinose; D-Galacturonic Acid; D-Glucosamine; D- |
| | | Glucuronic Acid; D-Ribose; L-Fucose; Lactulose; Laminarin; N-Acetyl-D- |
| | | Glucosamine; Pectin; a-D-Lactose; b-Methyl-D-Galactoside |
| FBI00070 | 1, 2 | 3-0-b-D-Galacto-pyranosyl-D-Arabinose; Amygdalin; Arbutin; Chondroitin |
| | | Sulfate C; D-Arabinose; D-Cellobiose; D-Fructose; D-Fructose-6-Phosphate; |
| | | D-Galactose; D-Galacturonic Acid; D-Glucosamine; D-Glucose-1-Phosphate; |
| | | D-Glucose-6-Phosphate; D-Glucuronic Acid; D-Mannose; D-Melezitose; D- |
| | | Melibiose; D-Raffinose; D-Ribose; D-Trehalose; D-Xylose; Dextrin; |
| | | Gentiobiose; Glycogen; L-Arabinose; L-Fucose; L-Galactonic Acid-g- |
| | | Lactone; L-Rhamnose; Lactitol; Lactulose; Maltitol; Maltose; Maltotriose; N- |
| | | Acetyl-D-Galactosamine; N-Acetyl-D-Glucosamine; N-Acetyl-b-D- |
| | | Mannosamine; Palatinose; Pectin; Salicin; Stachyose; Sucrose; Thymidine; |
| | | Turanose; Uridine; a-Cyclodextrin; a-D-Glucose; a-D-Lactose; a-Methyl-D- |
| | | Galactoside; a-Methyl-D-Glucoside; a-Methyl-D-Mannoside; b-Cyclodextrin; |
| | | b-Methyl-D-Galactoside; b-Methyl-D-Glucoside; g-Cyclodextrin |
| FBI00078 | 1, 2 | D-Arabinose; D-Cellobiose; D-Fructose; D-Galactose; D-Mannose; D- |
| | | Melibiose; D-Raffinose; D-Ribose; D-Sorbitol; D-Xylose; L-Arabinose; L- |
| | | Fucose; L-Lyxose; L-Rhamnose; L-Sorbose; Lactitol; Lactulose; Maltose; |
| | | Maltotriose; Pectin; Sedoheptulosan; Stachyose; Sucrose; Xylitol; a-D- |
| | | Glucose; a-D-Lactose; b-Methyl-D-Galactoside |
| FBI00079 | 1, 2 | 5-Keto-D-Gluconic Acid; Amygdalin; Arbutin; D-Cellobiose; D-Fructose; D- |
| | | Galactonic Acid-g-Lactone; D-Galactose; D-Gluconic Acid; D-Glucosamine; |
| | | D-Glucose-1-Phosphate; D-Glucuronic Acid; D-Mannose; D-Ribono-1,4- |
| | | Lactone; D-Ribose; D-Saccharic Acid; D-Xylose; Gentiobiose; L-Arabinose; |
| | | L-Fucose; L-Galactonic Acid-g-Lactone; Lactulose; Maltose; Maltotriose; |
| | | Mucic Acid; N-Acetyl-D-Glucosamine; N-Acetyl-Neuraminic Acid; N- |
| | | Acetyl-b-D-Mannosamine; Pectin; Salicin; Sucrose; Thymidine; Uridine; a-D- |
| | | Glucose; a-D-Lactose; b-Methyl-D-Galactoside; b-Methyl-D-Glucoside |
| FBI00087 | 1, 2 | D-Arabitol; D-Fructose; D-Galactose; D-Gluconic Acid; D-Ribose; D- |
| | | Sorbitol; D-Xylose; L-Arabinose; Sucrose; a-D-Glucose |
| FBI00102 | 1, 2 | Amygdalin; Chondroitin Sulfate C; D-Arabinose; D-Cellobiose; D-Fructose; |
| | | D-Galactose; D-Galacturonic Acid; D-Glucosamine; D-Glucuronic Acid; D- |
| | | Mannose; D-Melibiose; D-Raffinose; D-Ribose; D-Trehalose; D-Xylose; |
| | | Dextrin; Gentiobiose; Glycogen; Inulin; L-Arabinose; L-Fucose; L-Galactonic |
| | | Acid-g-Lactone; L-Rhamnose; Lactulose; Laminarin; Maltitol; Maltose; |
| | | Maltotriose; N-Acetyl-D-Galactosamine; N-Acetyl-D-Glucosamine; N-Acetyl- |
| | | b-D-Mannosamine; Palatinose; Pectin; Salicin; Sucrose; Turanose; Uridine; a- |
| | | Cyclodextrin; a-D-Glucose; a-D-Lactose; a-Methyl-D-Glucoside; a-Methyl-D- |
| | | Mannoside; b-Cyclodextrin; b-Methyl-D-Galactoside; g-Cyclodextrin |
| FBI00104 | 1, 2 | Amygdalin; D-Arabinose; D-Cellobiose; D-Fructose; D-Galactose; D- |
| | | Glucosamine; D-Melibiose; D-Raffinose; D-Sorbitol; D-Xylose; L-Arabinose; |
| | | L-Fucose; L-Rhamnose; Maltose; N-Acetyl-Neuraminic Acid; Stachyose; |
| | | Uridine; a-D-Glucose; a-D-Lactose |
| FBI00109 | 1, 2 | Arbutin; D-Fructose; D-Galactose; D-Melibiose; D-Raffinose; D-Sorbitol; |
| | | Maltose; Maltotriose; Salicin; Stachyose; a-D-Glucose; a-D-Lactose; b- |
| | | Methyl-D-Glucoside |
| FBI00110 | 1, 2 | 5-Keto-D-Gluconic Acid; Arbutin; D-Cellobiose; D-Fructose; D-Fructose-6- |
| | | Phosphate; D-Galactose; D-Gluconic Acid; D-Glucosamine; D-Mannose; D- |
| | | Melezitose; D-Raffinose; D-Ribose; D-Trehalose; D-Xylose; Gentiobiose; |
| | | Inosine; L-Arabinose; L-Fucose; Lactulose; Maltose; Maltotriose; N-Acetyl- |
| | | D-Glucosamine; Palatinose; Salicin; Stachyose; Sucrose; Thymidine; |
| | | Turanose; a-D-Glucose; b-D-Allose; b-Methyl-D-Glucoside |
| FBI00113 | 1, 2 | Arbutin; N-Acetyl-D-Galactosamine; a-Methyl-D-Galactoside |
| FBI00115 | 1, 2 | D-Fructose; D-Galactose; D-Xylose; L-Arabinose; Maltose; Maltotriose; N- |
| | | Acetyl-Neuraminic Acid; Turanose; a-D-Glucose |
| FBI00117 | 1, 2 | Arbutin; Maltotriose |
| FBI00125 | 1, 2 | Chondroitin Sulfate C; D-Fructose; D-Galactose; D-Glucosamine; D- |
| | | Mannose; Gentiobiose; Lactulose; Laminarin; Maltose; Maltotriose; N-Acetyl- |
| | | D-Galactosamine; N-Acetyl-D-Glucosamine; Pectin; Sucrose; a-Cyclodextrin; |
| | | a-D-Glucose; b-Cyclodextrin; b-Methyl-D-Galactoside; g-Cyclodextrin |
| FBI00128 | 1, 2 | 1,2-Propanediol; D-Fructose-6-Phosphate; a-D-Glucose |
| FBI00137 | 1, 2 | D-Fructose; D-Galactose; D-Glucosamine; D-Glucuronic Acid; D-Mannose; |
| | | D-Melibiose; D-Raffinose; D-Xylose; Dextrin; Glycogen; Inulin; L-Fucose; |
| | | Lactitol; Lactulose; Maltose; Maltotriose; N-Acetyl-D-Galactosamine; N- |
| | | Acetyl-D-Glucosamine; N-Acetyl-Neuraminic Acid; Sucrose; a-Cyclodextrin; |
| | | a-D-Glucose; a-D-Lactose; b-Cyclodextrin; b-Methyl-D-Galactoside; g- |
| | | Cyclodextrin |
| FBI00147 | 1, 2 | 2-Deoxy-D-Ribose; D-Fructose; D-Galactose; D-Gluconic Acid; D- |
| | | Glucosamine; D-Glucuronic Acid; D-Mannose; D-Melibiose; D-Psicose; D- |
| | | Raffinose; D-Ribose; D-Sorbitol; D-Trehalose; D-Xylose; L-Arabinose; |
| | | Lactulose; Maltose; Maltotriose; N-Acetyl-D-Glucosamine; N-Acetyl- |
| | | Neuraminic Acid; Palatinose; Stachyose; Sucrose; Thymidine; Uridine; a-D- |
| | | Glucose |
| FBI00165 | 1, 2 | N-Acetyl-D-Galactosamine; N-Acetyl-D-Glucosamine; N-Acetyl-Neuraminic |
| | | Acid |
| FBI00167 | 1, 2 | Amygdalin; Arbutin; D-Cellobiose; D-Fructose; D-Galactose; D-Glucosamine; |
| | | D-Raffinose; D-Sorbitol; Gentiobiose; L-Arabinose; Lactulose; Maltose; |
| | | Maltotriose; N-Acetyl-D-Galactosamine; N-Acetyl-Neuraminic Acid; Salicin; |
| | | Stachyose; Sucrose; Thymidine; Uridine; a-D-Glucose; a-D-Lactose; b- |
| | | Methyl-D-Glucoside; m-Inositol |
| FBI00174 | 1, 2 | Adenosine; D-Fructose-6-Phosphate; D-Galacturonic Acid; D-Gluconic Acid; |
| | | D-Glucose-1-Phosphate; D-Glucuronic Acid; D-Melezitose; D-Trehalose; |
| | | Inosine; L-Fucose; L-Galactonic Acid-g-Lactone; Laminarin; N-Acetyl-D- |
| | | Glucosamine; Pectin |
| FBI00180 | 1, 2 | Inulin; b-D-Allose |
| FBI00182 | 1, 2 | Amygdalin; Arbutin; D-Cellobiose; D-Fructose; D-Galactose; D-Galacturonic |
| | | Acid; D-Mannose; D-Melibiose; D-Raffinose; Dextrin; Dihydroxy Acetone; |
| | | Gentiobiose; L-Galactonic Acid-g-Lactone; L-Rhamnose; Lactulose; Maltose; |
| | | Maltotriose; N-Acetyl-D-Glucosamine; Pectin; Salicin; Stachyose; Sucrose; a- |
| | | D-Glucose; a-D-Lactose; a-Methyl-D-Galactoside; b-Methyl-D-Galactoside; |
| | | g-Cyclodextrin |
| FBI00184 | 1, 2 | Amygdalin; D-Galactose; D-Glucosamine; D-Mannose; D-Melibiose; D- |
| | | Raffinose; D-Trehalose; Gentiobiose; Lactulose; Maltitol; Maltose; |
| | | Maltotriose; N-Acetyl-D-Galactosamine; N-Acetyl-D-Glucosamine; N-Acetyl- |
| | | b-D-Mannosamine; Palatinose; Salicin; Stachyose; Sucrose; Turanose; |
| | | Uridine; a-Cyclodextrin; a-D-Glucose; a-D-Lactose; a-Methyl-D-Glucoside; b- |
| | | Cyclodextrin; b-Methyl-D-Galactoside; g-Cyclodextrin |
| FBI00189 | 1, 2 | Amygdalin; Arbutin; D-Arabinose; D-Cellobiose; D-Galactose; D- |
| | | Glucosamine; D-Mannitol; D-Mannose; D-Melibiose; D-Raffinose; D- |
| | | Sorbitol; D-Trehalose; Gentiobiose; Glycogen; L-Arabinose; L-Fucose; |
| | | Lactitol; Lactulose; Maltitol; Maltose; Maltotriose; N-Acetyl-D- |
| | | Galactosamine; N-Acetyl-D-Glucosamine; Palatinose; Salicin; Stachyose; |
| | | Sucrose; Turanose; a-Cyclodextrin; a-D-Glucose; a-D-Lactose; a-Methyl-D- |
| | | Glucoside; a-Methyl-D-Mannoside; b-Cyclodextrin; b-Methyl-D-Galactoside; |
| | | b-Methyl-D-Glucoside; g-Cyclodextrin |
| FBI00190 | 1, 2 | Amygdalin; Arbutin; Chondroitin Sulfate C; D-Arabinose; D-Cellobiose; D- |
| | | Fructose; D-Fructose-6-Phosphate; D-Galactose; D-Galacturonic Acid; D- |
| | | Glucosamine; D-Glucose-1-Phosphate; D-Glucose-6-Phosphate; D-Glucuronic |
| | | Acid; D-Mannose; D-Melezitose; D-Melibiose; D-Raffinose; D-Trehalose; D- |
| | | Xylose; Dextrin; Gentiobiose; Glycogen; Glycyl-L-Aspartic Acid; L- |
| | | Arabinose; L-Fucose; L-Galactonic Acid-g-Lactone; L-Rhamnose; Lactitol; |
| | | Lactulose; Laminarin; Maltitol; Maltose; Maltotriose; N-Acetyl-D- |
| | | Galactosamine; N-Acetyl-D-Glucosamine; N-Acetyl-b-D-Mannosamine; |
| | | Palatinose; Pectin; Salicin; Stachyose; Sucrose; Thymidine; Turanose; |
| | | Uridine; a-D-Glucose; a-D-Lactose; a-Methyl-D-Galactoside; a-Methyl-D- |
| | | Glucoside; a-Methyl-D-Mannoside; b-Methyl-D-Galactoside; b-Methyl-D- |
| | | Glucoside |
| FBI00191 | 1, 2 | D-Trehalose; L-Arabinose; Maltotriose; N-Acetyl-D-Glucosamine; a- |
| | | Cyclodextrin; b-Cyclodextrin |
| FBI00194 | 1, 2 | Arbutin; D-Arabinose; D-Fructose; D-Galactose; D-Glucosamine; D- |
| | | Melibiose; D-Raffinose; D-Sorbitol; D-Trehalose; Gentiobiose; L-Fucose; |
| | | Lactulose; Maltose; Maltotriose; N-Acetyl-D-Glucosamine; Salicin; |
| | | Stachyose; Sucrose; Turanose; a-D-Glucose; a-D-Lactose |
| FBI00198 | 1, 2 | 2-Deoxy Adenosine; Arbutin; D-Cellobiose; D-Fructose; D-Galactose; D- |
| | | Galacturonic Acid; D-Gluconic Acid; D-Glucosamine; D-Glucuronic Acid; D- |
| | | Mannose; D-Melezitose; D-Melibiose; D-Raffinose; D-Ribose; D-Trehalose; |
| | | D-Xylose; Inosine; L-Arabinose; L-Fucose; Lactulose; Maltose; Maltotriose; |
| | | N-Acetyl-D-Glucosamine; N-Acetyl-Neuraminic Acid; Palatinose; Stachyose; |
| | | Thymidine; Turanose; a-D-Glucose; a-D-Lactose; b-D-Allose |
| FBI00199 | 1, 2 | D-Fructose; D-Galactonic Acid-g-Lactone; D-Galactose; D-Gluconic Acid; D- |
| | | Glucosamine; D-Glucose-1-Phosphate; D-Glucose-6-Phosphate; D-Mannose; |
| | | D-Melibiose; D-Psicose; D-Raffinose; D-Ribose; D-Sorbitol; D-Trehalose; D- |
| | | Xylose; L-Arabinose; L-Serine; L-Tartaric Acid; Lactulose; Maltose; |
| | | Maltotriose; N-Acetyl-D-Glucosamine; N-Acetyl-Neuraminic Acid; |
| | | Palatinose; Stachyose; Sucrose; Uridine; a-D-Glucose |
| FBI00200 | 1, 2 | D-Galacturonic Acid; D-Gluconic Acid; D-Glucuronic Acid; L-Galactonic |
| | | Acid-g-Lactone; Pectin |
| FBI00201 | 1, 2 | Dextrin; Glycogen; Inulin; Laminarin; g-Cyclodextrin |
| FBI00205 | 1, 2 | 3-0-b-D-Galacto-pyranosyl-D-Arabinose; Arbutin; D-Arabinose; D-Fructose; |
| | | D-Galactose; D-Gluconic Acid; D-Mannitol; D-Melibiose; D-Raffinose; D- |
| | | Sorbitol; D-Xylose; L-Fucose; L-Rhamnose; L-Tartaric Acid; Lactitol; |
| | | Lactulose; Maltitol; Maltose; Maltotriose; Melibionic Acid; N-Acetyl-D- |
| | | Glucosamine; Palatinose; Pectin; Salicin; Stachyose; Sucrose; Turanose; a-D- |
| | | Glucose; a-D-Lactose; a-Methyl-D-Galactoside; b-Methyl-D-Galactoside; m- |
| | | Inositol |
| FBI00220 | 1, 2 | D-Arabitol; D-Fructose; D-Galacturonic Acid; D-Gluconic Acid; D-Mannitol; |
| | | D-Xylose; L-Serine; Maltose; Maltotriose; Pyruvic Acid; Sucrose; a-D- |
| | | Glucose |
| FBI00232 | 1, 2 | Chondroitin Sulfate C; D-Galactose; Glycogen; Inulin; N-Acetyl-D- |
| | | Galactosamine; N-Acetyl-D-Glucosamine; Pectin; Sucrose; a-Cyclodextrin; b- |
| | | Cyclodextrin; g-Cyclodextrin |
| FBI00235 | 1, 2 | 1,2-Propanediol; 2-Hydroxy Benzoic Acid; 5-Keto-D-Gluconic Acid; Acetic |
| | | Acid; Amygdalin; D-Arabinose; D-Fucose; D-Ribose; D-Tagatose; D- |
| | | Threonine; L-Arabitol; L-Arginine; L-Pyroglutamic Acid; L-Rhamnose; |
| | | Maltitol; N-Acetyl-L-Glutamic Acid; Oxalic Acid; Quinic Acid; Sebacic Acid; |
| | | Stachyose; Succinamic Acid; Turanose; a-D-Lactose; a-Hydroxy Glutaric |
| | | Acid-g-Lactone; a-Keto-Butyric Acid; a-Keto-Valeric Acid; a-Methyl-D- |
| | | Galactoside; a-Methyl-D-Glucoside; a-Methyl-D-Mannoside; b-Methyl-D- |
| | | Xyloside; d-Amino Valeric Acid; g-Amino Butyric Acid; g-Hydroxy Butyric |
| | | Acid |
| FBI00236 | 1, 2 | D-Galactose; D-Mannose; D-Melibiose; D-Raffinose; D-Trehalose; L-Fucose; |
| | | Maltose; Maltotriose; N-Acetyl-D-Glucosamine; Stachyose; Turanose; a- |
| | | Methyl-D-Galactoside; b-Methyl-D-Galactoside; b-Methyl-D-Glucoside; b- |
| | | Methyl-D-Xyloside |
| FBI00245 | 1, 2 | Amygdalin; Chondroitin Sulfate C; D-Arabinose; D-Cellobiose; D-Fructose; |
| | | D-Galactose; D-Galacturonic Acid; D-Gluconic Acid; D-Glucosamine; D- |
| | | Glucuronic Acid; D-Mannose; D-Melezitose; D-Melibiose; D-Raffinose; D- |
| | | Ribose; D-Saccharic Acid; D-Trehalose; D-Xylose; Dextrin; Gentiobiose; |
| | | Glycogen; Inulin; L-Arabinose; L-Fucose; L-Galactonic Acid-g-Lactone; L- |
| | | Glutamine; L-Histidine; L-Pyroglutamic Acid; L-Rhamnose; Lactitol; |
| | | Lactulose; Maltitol; Maltose; Maltotriose; Mannan; Mucic Acid; N-Acetyl-D- |
| | | Galactosamine; N-Acetyl-D-Glucosamine; N-Acetyl-b-D-Mannosamine; |
| | | Palatinose; Pectin; Pyruvic Acid; Salicin; Stachyose; Sucrose; Turanose; |
| | | Uridine; a-Cyclodextrin; a-D-Glucose; a-D-Lactose; a-Keto-Butyric Acid; a- |
| | | Keto-Glutaric Acid; a-Methyl-D-Galactoside; a-Methyl-D-Glucoside; a- |
| | | Methyl-D-Mannoside; b-Cyclodextrin; b-Methyl-D-Galactoside; b-Methyl-D- |
| | | Glucoside; g-Cyclodextrin |
| FBI00263 | 1, 2 | D-Galactose; D-Glucosamine; D-Mannose; D-Melibiose; D-Raffinose; D- |
| | | Trehalose; L-Arabinose; L-Fucose; Lactulose; Maltitol; Maltose; N-Acetyl-D- |
| | | Galactosamine; N-Acetyl-D-Glucosamine; N-Acetyl-Neuraminic Acid; |
| | | Stachyose; Sucrose; Turanose; a-D-Glucose; a-D-Lactose; a-Methyl-D- |
| | | Glucoside; b-Methyl-D-Galactoside |
| FBI00269 | 1, 2 | D,L-a-Glycerol-Phosphate; D-Gluconic Acid; D-Glucuronic Acid; Dextrin; |
| | | Gelatin; Glycogen; Inulin; Laminarin; g-Cyclodextrin |
| FBI00278 | 1, 2 | Glycogen; Thymidine |
| FBI00281 | 1, 2 | D-Ribose; N-Acetyl-Neuraminic Acid |
| FBI00290 | 1, 2 | Glycogen |
| FBI00009 | 2 | Acetamide; D-Raffinose; g-Cyclodextrin |
| FBI00011 | 2 | 4-Hydroxy Benzoic Acid; Capric Acid; D,L-Carnitine; D,L-Octopamine; D- |
| | | Glucosamine; D-Ribono-1,4-Lactone; Dihydroxy Acetone; Glycine; Inulin; L- |
| | | Alaninamide; L-Arginine; L-Histidine; L-Homoserine; N-Acetyl-L-Glutamic |
| | | Acid; Putrescine; Quinic Acid; Turanose |
| FBI00016 | 2 | D-Arabinose; D-Glucosamine; D-Raffinose; Dextrin; Glycogen; Lactitol; N- |
| | | Acetyl-D-Galactosamine; N-Acetyl-Neuraminic Acid; Palatinose; Stachyose; |
| | | a-Cyclodextrin; b-Cyclodextrin; b-Methyl-D-Galactoside; g-Cyclodextrin |
| FBI00020 | 2 | Amygdalin; D-Arabinose; D-Glucosamine; D-Melezitose; D-Raffinose; |
| | | Gentiobiose; Maltitol; N-Acetyl-D-Galactosamine; Palatinose; Salicin; |
| | | Stachyose; a-Methyl-D-Glucoside; a-Methyl-D-Mannoside; b-Methyl-D- |
| | | Galactoside; g-Cyclodextrin |
| FBI00021 | 2 | Amygdalin; Arbutin; D-Arabinose; D-Arabitol; D-Glucosamine; D- |
| | | Melezitose; D-Raffinose; Gentiobiose; Glycogen; Lactitol; Maltitol; N-Acetyl- |
| | | D-Galactosamine; Palatinose; Pectin; Salicin; Stachyose; Turanose; a- |
| | | Cyclodextrin; a-Methyl-D-Glucoside; a-Methyl-D-Mannoside; b- |
| | | Cyclodextrin; b-Methyl-D-Galactoside; g-Cyclodextrin |
| FBI00029 | 2 | Amygdalin; Arbutin; D-Glucosamine; D-Melezitose; D-Raffinose; Dextrin; |
| | | Gentiobiose; Inulin; Lactitol; Laminarin; Maltitol; N-Acetyl-D-Galactosamine; |
| | | N-Acetyl-Neuraminic Acid; Palatinose; Salicin; Stachyose; Turanose; a- |
| | | Cyclodextrin; a-Methyl-D-Glucoside; a-Methyl-D-Mannoside; b- |
| | | Cyclodextrin; b-Methyl-D-Galactoside; g-Cyclodextrin |
| FBI00032 | 2 | 5-Keto-D-Gluconic Acid; Arbutin; D-Glucosamine; Inulin; L-Sorbose; L- |
| | | Tartaric Acid; Lactitol; Maltitol; N-Acetyl-D-Glucosaminitol; Palatinose; |
| | | Pectin; Salicin; Turanose; a-Methyl-D-Mannoside |
| FBI00043 | 2 | 2-Hydroxy Benzoic Acid; Amygdalin; D-Arabinose; D-Ribono-1,4-Lactone; |
| | | Dextrin; Dihydroxy Acetone; Glycine; L-Leucine; Maltitol; N-Acetyl- |
| | | Neuraminic Acid; Oxalomalic Acid; Palatinose; Quinic Acid; Sebacic Acid; |
| | | Stachyose; a-Methyl-D-Glucoside; b-Methyl-D-Galactoside |
| FBI00049 | 2 | b-Cyclodextrin; g-Cyclodextrin |
| FBI00052 | 2 | Amygdalin; Arbutin; D-Arabinose; D-Glucosamine; D-Melezitose; D- |
| | | Raffinose; Gentiobiose; Maltitol; N-Acetyl-D-Galactosamine; Palatinose; |
| | | Salicin; Turanose; a-Methyl-D-Glucoside; a-Methyl-D-Mannoside; b-Methyl- |
| | | D-Galactoside |
| FBI00056 | 2 | Amygdalin; D-Raffinose; Dextrin; Gentiobiose; Maltitol; N-Acetyl- |
| | | Neuraminic Acid; Palatinose; Stachyose; Turanose; b-Methyl-D-Galactoside |
| FBI00060 | 2 | Amygdalin; Arbutin; D-Arabinose; D-Glucosamine; D-Melezitose; D- |
| | | Raffinose; Dextrin; Gentiobiose; Glycogen; Lactitol; Maltitol; N-Acetyl-D- |
| | | Galactosamine; Palatinose; Salicin; Turanose; a-Cyclodextrin; a-Methyl-D- |
| | | Glucoside; a-Methyl-D-Mannoside; b-Cyclodextrin; b-Methyl-D-Galactoside; |
| | | g-Cyclodextrin |
| FBI00062 | 2 | Dextrin; Lactitol; Maltitol; Palatinose; Salicin; Stachyose; Turanose; b- |
| | | Methyl-D-Galactoside; g-Cyclodextrin |
| FBI00075 | 2 | b-Methyl-D-Galactoside |
| FBI00076 | 2 | Amygdalin; D-Arabinose; D-Glucosamine; D-Melezitose; D-Raffinose; |
| | | Gentiobiose; Glycogen; Inulin; Laminarin; Maltitol; N-Acetyl-D- |
| | | Galactosamine; Palatinose; Pectin; Salicin; Turanose; a-Methyl-D-Glucoside; |
| | | a-Methyl-D-Mannoside; b-Methyl-D-Galactoside; g-Cyclodextrin |
| FBI00080 | 2 | Amygdalin; Arbutin; D-Melezitose; D-Raffinose; Dextrin; L-Arginine; |
| | | Laminarin; Maltitol; N-Acetyl-Neuraminic Acid; Pectin; Stachyose; a-Methyl- |
| | | D-Mannoside; b-Cyclodextrin; b-Methyl-D-Galactoside |
| FBI00111 | 2 | D-Arabinose; D-Glucosamine; D-Raffinose; Stachyose; b-D-Allose |
| FBI00112 | 2 | Amygdalin; Arbutin; D-Arabinose; D-Glucosamine; Inulin; N-Acetyl-D- |
| | | Galactosamine; Salicin; a-Cyclodextrin; b-Cyclodextrin; b-Methyl-D- |
| | | Galactoside; g-Cyclodextrin |
| FBI00116 | 2 | Amygdalin; D-Arabinose; D-Glucosamine; D-Raffinose; Gentiobiose; |
| | | Palatinose; Pectin; Stachyose; Turanose |
| FBI00124 | 2 | D-Glucosamine; D-Raffinose; Lactitol; Pectin; Stachyose; a-Methyl-D- |
| | | Mannoside |
| FBI00126 | 2 | Arbutin; D-Glucosamine; D-Tagatose; N-Acetyl-D-Galactosamine; Pectin |
| FBI00127 | 2 | Amygdalin; Arbutin; Lactitol; Pectin; Salicin; Turanose; b-Methyl-D- |
| | | Galactoside |
| FBI00135 | 2 | Amygdalin; D-Glucosamine; N-Acetyl-D-Galactosamine; N-Acetyl- |
| | | Neuraminic Acid |
| FBI00140 | 2 | Dextrin; Gentiobiose; Glycogen; Laminarin; Stachyose; a-Cyclodextrin; b- |
| | | Cyclodextrin; g-Cyclodextrin |
| FBI00145 | 2 | Amygdalin; Arbutin; D-Raffinose; Dextrin; Gentiobiose; Lactitol; Salicin; |
| | | Stachyose; b-Methyl-D-Galactoside; g-Cyclodextrin |
| FBI00151 | 2 | 2-Deoxy-D-Ribose; D-Arabinose; D-Fructose; D-Gluconic Acid; D- |
| | | Glucosamine; D-Mannose; D-Raffinose; Dextrin; Gentiobiose; Glycogen; L- |
| | | Isoleucine; Maltitol; Maltose; Maltotriose; Mannan; N-Acetyl-D- |
| | | Glucosamine; Palatinose; Pectin; Stachyose; Sucrose; Turanose; a- |
| | | Cyclodextrin; a-D-Glucose; b-Cyclodextrin; b-D-Allose; b-Methyl-D- |
| | | Galactoside; g-Cyclodextrin |
| FBI00152 | 2 | D-Raffinose; Dextrin; Gentiobiose; Glycogen; Laminarin; Stachyose; a- |
| | | Cyclodextrin; b-Cyclodextrin; g-Cyclodextrin |
| FBI00175 | 2 | D-Arabinose; D-Arabitol |
| FBI00197 | 2 | Amygdalin; Arbutin; Chondroitin Sulfate C; D-Arabinose; D-Glucosamine; D- |
| | | Melezitose; D-Raffinose; Gentiobiose; Lactitol; Laminarin; N-Acetyl-D- |
| | | Galactosamine; N-Acetyl-Neuraminic Acid; Pectin; Salicin; Stachyose; |
| | | Turanose; a-Cyclodextrin; b-Cyclodextrin; b-Methyl-D-Galactoside; b- |
| | | Methyl-D-Glucuronic Acid; b-Methyl-D-Xyloside; g-Cyclodextrin |
| FBI00206 | 2 | Amygdalin; Arbutin; D-Arabinose; D-Glucosamine; D-Melezitose; D- |
| | | Raffinose; Dextrin; Gentiobiose; Glycogen; Inulin; Lactitol; Maltitol; N- |
| | | Acetyl-D-Galactosamine; Palatinose; Pectin; Salicin; Stachyose; Turanose; a- |
| | | Cyclodextrin; a-Methyl-D-Glucoside; a-Methyl-D-Mannoside; b- |
| | | Cyclodextrin; b-Methyl-D-Galactoside; g-Cyclodextrin |
| FBI00211 | 2 | Amygdalin; D-Raffinose; Glycogen; Lactitol; Maltitol; Palatinose; Turanose; |
| | | b-Methyl-D-Galactoside |
| FBI00212 | 2 | 3-Methyl Glucose; 4-Hydroxy Benzoic Acid; Amygdalin; Arbutin; D- |
| | | Arabinose; D-Arabitol; D-Melezitose; Dextrin; Glycogen; Inulin; L- |
| | | Isoleucine; L-Lysine; L-Methionine; Lactitol; Maltitol; Palatinose; Turanose; |
| | | a-Methyl-D-Glucoside; b-D-Allose; b-Methyl-D-Galactoside; g-Cyclodextrin; |
| | | i-Erythritol |
| FBI00243 | 2 | Sec-Butylamine |
| FBI00251 | 2 | D-Raffinose; Dextrin; Glycogen; Lactitol; Palatinose; Salicin; Turanose; b- |
| | | Methyl-D-Galactoside; g-Cyclodextrin |
| FBI00255 | 2 | Arbutin; D-Arabinose; D-Melezitose; D-Raffinose; Inulin; Stachyose; b- |
| | | Cyclodextrin; b-Methyl-D-Glucuronic Acid; b-Methyl-D-Xyloside; g- |
| | | Cyclodextrin |
| FBI00267 | 2 | D,L-Octopamine; Sec-Butylamine |
| FBI00271 | 2 | 3-Methyl Glucose; Arbutin; D,L-Carnitine; D-Arabinose; D-Arabitol; D- |
| | | Raffinose; D-Ribono-1,4-Lactone; D-Tartaric Acid; Gentiobiose; Glycogen; |
| | | Hydroxy-L-Proline; Itaconic Acid; L-Histidine; L-Isoleucine; L-Valine; |
| | | Laminarin; Quinic Acid; Sebacic Acid; Sorbic Acid; Stachyose; Succinamic |
| | | Acid; b-Hydroxy Butyric Acid; b-Methyl-D-Galactoside |
| FBI00274 | 2 | D-Ribono-1,4-Lactone; L-Histidine; L-Homoserine; Quinic Acid |

The single carbon source conditions of Biolog plates are restrictive and not expected to promote growth of strains with more complex growth requirements. This was observed with the 41 strains of FB-001 that do not have a positive growth signature in the Biolog assays performed and the 9 strains that were not tested using Biolog due to insufficient growth. Of these combined 50 strains, 23 were tested with just 2 plates, 18 were tested with both PM1 and 2 plates, and 9 fastidious strains failed to reach the turbidity necessary to conduct a Biolog assay and are characterized in more detail below. The 41 strains that reached sufficient growth OD in complex growth media, but did not show positive growth in the Biolog plates tested, are shown in Table 35. These strains are routinely grown on complex YCFAC media and growth data in this medium are provided as the OD600 reached in the time given. Further information on the cultivation of these strains is available in the primary literature and summarized in Table 35 as well. In brief, for each strain we provide known macronutrient utilization, metabolite production and oxalate-formate characters. Macronutrients describe the primary contributors to biomass for a given strain, whereas metabolite production describes excreted small molecules that accumulate during cultivation and oxalate-formate focuses on the ability to degrade or resist the presence of these molecules. In cases where a macronutrient is predicted to be a substrate for growth, but growth was not directly observed in Biolog assays, it is expected that a second nutrient is required such as a vitamin or alternative nitrogen source that can be provided by the YCFAC recipe used for routine growth.

TABLE 35
Characterization of the 41 strains that did not show positive growth signatures by Biolog assay

| | | YCFAC | Incubation | |
|---|---|---|---|---|
| Strain | PM | OD₆₀₀ | time (h) | Macronutrient |
| FBI00068 | 1, 2 | 0.573 | 72 | mucin o-linked glycans |
| FBI00012 | 1, 2 | 0.788 | 68 | indole; mannose; raffinose |
| FBI00277 | 1, 2 | 1.097 | 68 | indole; mannose; raffinose |
| FBI00022 | 1, 2 | 0.342 | 72 | N/A |
| FBI00229 | 1, 2 | 0.186 | 72 | mannose |
| FBI00061 | 1, 2 | 0.047 | 72 | mannose; raffinose |
| FBI00238 | 1, 2 | 0.251 | 72 | N/A |
| FBI00288 | 1, 2 | 0.877 | 66.6 | N/A |
| FBI00038 | 1, 2 | 0.512 | 72 | acetate; cellobiose; fructose; glucose; |
| | | | | lactose; maltose; mannose; raffinose; |
| | | | | starch |
| FBI00170 | 1, 2 | 0.067 | 72 | acetate; arginine; secoisolariciresinol |
| | | | | diglucoside |
| FBI00096 | 1, 2 | 0.068 | 72 | acetate; arginine; secoisolariciresinol |
| | | | | diglucoside |
| FBI00159 | 1, 2 | 0.063 | 68 | N/A |
| FBI00018 | 1, 2 | 0.799 | 72 | acetate; arabinoxylan; arginine; inulin; |
| | | | | starch; xos |
| FBI00099 | 1, 2 | 0.094 | 72 | arginine; methionine |
| FBI00081 | 1, 2 | 0.612 | 72 | N/A |
| FBI00071 | 1, 2 | 1.066 | 70.7 | N/A |
| FBI00097 | 1, 2 | 0.179 | 72 | N/A |
| FBI00233 | 1, 2 | 0.432 | 72 | N/A |
| FBI00019 | 2 | 0.12 | 72 | indole |
| FBI00208 | 2 | 0.768 | 70 | inulin; mannose |
| FBI00162 | 2 | 1.146 | 70 | N/A |
| FBI00171 | 2 | 0.164 | 72 | N/A |
| FBI00221 | 2 | 0.963 | 70 | glucose |
| FBI00226 | 2 | 0.097 | 72 | arabinose; glucose; mannose; xylose |
| FBI00040 | 2 | 0.056 | 72 | N/A |
| FBI00237 | 2 | 0.053 | 71.8 | N/A |
| FBI00248 | 2 | 1.13 | 71.8 | N/A |
| FBI00260 | 2 | 1.153 | 72 | acetate; arabinoxylan; arginine; inulin; |
| | | | | starch; xos |
| FBI00244 | 2 | 0.114 | 71.8 | acetate; arginine; chondroitin sulfate; |
| | | | | fructose; galacturonate; glucose; inulin; |
| | | | | maltose; starch |
| FBI00132 | 2 | 0.154 | 72 | arginine; methionine |
| FBI00120 | 2 | 0.735 | 72 | starch |
| FBI00092 | 2 | 0.15 | 87.8 | arabinose; xylose |
| FBI00149 | 2 | 0.157 | 87.8 | arabinose; xylose |
| FBI00177 | 2 | 0.082 | 87.8 | N/A |
| FBI00066 | 2 | 0.09 | 87.8 | N/A |
| FBI00093 | 2 | 0.896 | 71.8 | starch |
| FBI00123 | 2 | 1.4 | 71.8 | starch |
| FBI00069 | 2 | 0.11 | 72 | starch |
| FBI00085 | 2 | 0.733 | 72 | starch |
| FBI00224 | 2 | 0.047 | 70 | cysteine |
| FBI00077 | 2 | 0.062 | 72 | cysteine |

While most strains show a Biolog or YCFAC signature, the nine most fastidious strains require more characterization, which are provided here. Of these nine strains, two are isolates of Methanobrevibacter smithii (FBI00270 and FBI00292), the only archaeal strains in FB-001. M. smithii grows through methanogenesis (CH₄ production) with utilization of CO₂+H₂, or formate (HCO₂⁻) as macronutrients. Because of these specific growth conditions and phylogeny, M. smithii can be challenging to grow, but is readily identifiable. Another two strains are Oxalobacter formigenes strains FBI0133 and FBI0289, which can be readily grown with YCFAC supplemented with 20 mM Sodium oxalate.

Strain FBI00258 Turicibacter sanguinis, is most easily identified through its distinctive filamentous cell shape, with filamentous growth contributing to a lack of turbidity observed in dispersed culture. For strains FBI00254 Eubacterium hallii, FBI00034 Eubacterium eligens, FBI00176 Ruthenibacterium lactatiformans, and FBI00273 Barnesiella intestinihominis identification was conducted with differential plating on four recipes of complex media (Table 36).

TABLE 36
Seven-day growth scores for strains FBI00176 Ruthenibacterium
lactatiformans and FBI00273 Barnesiella intestinihominis

| Strain ID | YCFAC | YCFAC-B | BHI | CBA |
|---|---|---|---|---|
| FBI00273 | + | − | − | + |
| FBI00176 | + | − | + | + |
| FBI00254 | + | + | − | + |
| FBI00034 | + | + | − | + |

PM1 plates contained the following molecules: L-Arabinose; N-Acetyl-D-Glucosamine; D-Saccharic Acid; Succinic Acid; D-Galactose; L-Aspartic Acid; L-Proline; D-Alanine; D-Trehalose; D-Mannose; Dulcitol; D-Serine; D-Sorbitol; Glycerol; L-Fucose; D-Glucuronic Acid; D-Gluconic Acid; D,L-a-Glycerol-Phosphate; D-Xylose; L-Lactic Acid; Formic Acid; D-Mannitol; L-Glutamic Acid; D-Glucose-6-Phosphate; D-Galactonic Acid-g-Lactone; D,L-Malic Acid; D-Ribose; Tween 20; L-Rhamnose; D-Fructose; Acetic Acid; a-D; Glucose; Maltose; D-Melibiose; Thymidine; L-Asparagine; D-Aspartic Acid; D-Glucosaminic Acid; 1,2-Propanediol; Tween 40; a-Keto-Glutaric Acid; a-Keto-Butyric Acid; a-Methyl-D-Galactoside; a-D-Lactose; Lactulose; Sucrose; Uridine; L-Glutamine; m-Tartaric Acid; D-Glucose-1-Phosphate; D-Fructose-6-Phosphate; Tween 80; a-Hydroxy Glutaric Acid-g-Lactone; a-Hydroxy Butyric Acid; b-Methyl-D-Glucoside; Adonitol; Maltotriose; 2-Deoxy Adenosine; Adenosine; Glycyl-L-Aspartic Acid; Citric Acid; m-Inositol; D-Threonine; Fumaric Acid; Bromo Succinic Acid; Propionic Acid; Mucic Acid; Glycolic Acid; Glyoxylic Acid; D-Cellobiose; InosinevGlycyl-L-Glutamic Acid; Tricarballylic Acid; L-Serine; L-Threonine; L-Alanine; L-Alanyl-Glycine; Acetoacetic Acid; N-Acetyl-b-D-Mannosamine; Mono Methyl Succinate; Methyl Pyruvate; D-Malic Acid; L-Malic Acid; Glycyl-L-Proline; p-Hydroxy Phenyl Acetic Acid; m-Hydroxy Phenyl Acetic Acid; Tyramine; D-Psicose; L-Lyxose; Glucuronamide; Pyruvic Acid; L-Galactonic Acid-g-Lactone; D; Galacturonic Acid; Phenylethyl-amine; 2-Aminoethanol.

PM2 plates contained the following molecules: Chondroitin Sulfate C; a-Cyclodextrin; b-Cyclodextrin; g-Cyclodextrin; Dextrin; Gelatin; Glycogen; Inulin; Laminarin; Mannan; Pectin; N-Acetyl-D-Galactosamine; N-Acetyl-Neuraminic Acid; b-D-Allose; Amygdalin; D-Arabinose; D-Arabitol; L-Arabitol; Arbutin; 2-Deoxy-D-Ribose; i-Erythritol; D-Fucose; 3-0-b-D-Galacto-pyranosyl-D-Arabinose; Gentiobiose; L-Glucose; Lactitol; D-Melezitose; Maltitol; a-Methyl-D-Glucoside; b-Methyl-D-Galactoside; 3-Methyl Glucose; b-Methyl-D-Glucuronic Acid; a-Methyl-D-Mannoside; b-Methyl-D-Xyloside; Palatinose; D-Raffinose; Salicin; Sedoheptulosan; L-Sorbose; Stachyose; D-Tagatose; Turanose; Xylitol; N-Acetyl-D-Glucosaminitol; g-Amino Butyric Acid; d-Amino Valeric Acid; Butyric Acid; Capric Acid; Caproic Acid; Citraconic Acid; Citramalic Acid; D-Glucosamine; 2-Hydroxy Benzoic Acid; 4-Hydroxy Benzoic Acid; b-Hydroxy Butyric Acid; g-Hydroxy Butyric Acid; a-Keto-Valeric Acid; Itaconic Acid; 5-Keto-D-Gluconic Acid; D-Lactic Acid Methyl Ester; Malonic Acid; Melibionic Acid; Oxalic Acid; Oxalomalic Acid; Quinic Acid; D-Ribono-1,4-Lactone; Sebacic Acid; Sorbic Acid; Succinamic Acid; D-Tartaric Acid; L-Tartaric Acid; Acetamide; L-Alaninamide; N-Acetyl-L-Glutamic Acid; L-Arginine; Glycine; L-Histidine; L-Homoserine; Hydroxy-L-Proline; L-Isoleucine; L-Leucine; L-Lysine; L-Methionine; L-Ornithine; L-Phenylalanine; L-Pyroglutamic Acid; L-Valine; D,L-Carnitine; Sec-Butylamine; D.L-Octopamine; Putrescine; Dihydroxy Acetone; 2,3-Butanediol; 2,3-Butanedione; 3-Hydroxy 2-Butanone.

Preparation of cell suspension and PM MicroPlate Inoculation. AN IF-0a Inoculating Fluid (1.2×) was prepared by adding 1.5 ml of 1 M NaHCO₃, 0.15 ml of 0.4 M thioglycolate and 0.15 ml of 1 mM methylene green to a bottle of IF-0a GN/GP base inoculating fluid (1.2×), for a total of 125 ml AN IF-0a Inoculating Fluid (1.2×). The inoculating fluid is confirmed to be fully deoxygenated when colorless as the methylene green indicator changes from the oxidized (green) to the reduced (colorless) form. PM MicroPlates were removed from packaging, placed in an anaerobic chamber. And allowed to equilibrate to the oxygen-free gas mix (5% CO₂, 5% H₂, 90% N₂) for two days to become anaerobic. Preparation of PM inoculating fluids comprised: 1) Prepared a test tube containing 10 ml of 1.2×AN IF-0a, 2) Prepared inoculating fluids as described below, and 3) Dispensed inoculating fluids into vials.

Inoculation of PM MicroPlates. All the following steps were done in a strictly anaerobic atmosphere containing 5% CO₂, 5% H₂, 90% N₂. Step 1: Prepare Cell Suspensions (a. Strains were re-streaked from Research Cell Banks (RCBs) onto four plates of YCFAC media by streaking heavily and allowing the cells to grow 1-7 days at 37° C. in an atmosphere containing 5% CO₂, 5% H₂, 90% N₂; b.

Cells were harvested from agar plates using a sterile swab and transferred into a tube containing 10 ml of 1.2×AN IF-0a. Cell suspensions were gently stirred with the swab to obtain a uniform suspension.

Turbidity of the suspension was measured in Turbidimeter, and cells added to achieve a density of 40% T (transmittance)). Step 2: Inoculate PMs 1 and 2 (a. MicroPlates were prepped and labeled for each strain; b. 1.5 ml of cell suspension (Mix A) were added to 22.5 ml of AN PM1,2 inoculating fluid (Mix B) to a total of 24.0 ml. The final cell density is a 1:16 dilution of 40% T; c. PM MicroPlates were inoculated anaerobically from the 24 ml AB mixture by multichannel pipettor, with 100 ml aliquots per well).

Incubation and Data Collection. All cultures were maintained at 37° C. and anerobic conditions throughout the incubation. Growth of cells was measure by reading optical density at 600 nm (OD600) every 2 hours for using an Agilent Biostack microplate reader for 50-90 hours, depending on when stationary phase was reached across the plate.

While the present invention has been described at some length and with some particularity with respect to the several described embodiments, it is not intended that it should be limited to any such particulars or embodiments or any particular embodiment, but it is to be construed with references to the appended claims so as to provide the broadest possible interpretation of such claims in view of the prior art and, therefore, to effectively encompass the intended scope of the invention.

All publications, patent applications, patents, and other references mentioned herein are incorporated by reference in their entirety. In case of conflict, the present specification, including definitions, will control. In addition, section headings, the materials, methods, and examples are illustrative only and not intended to be limiting.

1. A composition comprising a microbial consortia comprising at least 1 oxalate-metabolizing microbial strain, wherein the at least one strain expresses an enzyme selected from a formyl-CoA transferase, an oxalate-formate antiporter, and an oxalyl-CoA decarboxylase, wherein the at least 1 oxalate-metabolizing microbial strain is from the Oxalobacter genus.

2. (canceled)

3. The composition of claim 1 comprising at least 3 oxalate-metabolizing microbial strains, wherein the at least 3 oxalate-metabolizing microbial strains are different strains of the same species or of different species.

4. (canceled)

5. The composition of claim 3, wherein the species is Oxalobacter formigenes (O. formigenes), and optionally wherein the number of oxalate-metabolizing microbial strains is 3 or more.

6. The composition of claim 3, wherein:
a) at least one strain is a low pH tolerance strain;
b) at least one strain is a high oxalate tolerance strain; and/or
c) at least one strain is a high growth rate strain.

7. A composition comprising at least 2 Oxalobacter formigenes (O. formigenes) strains, wherein each of the strains comprises one or more of the following functions:
a) a low pH tolerance strain;
b) a high oxalate tolerance strain; and/or
c) a high growth rate strain.

8. (canceled)

9. The composition of claim 6, wherein:
a) the low pH tolerance strain can metabolize oxalate at a pH between about 4 and about 6;
b) the high oxalate tolerance strain can metabolize oxalate at a concentration between about 5 mM to about 30 mM;
c) the low pH tolerance strain can metabolize oxalate at a pH of about 5; and/or
d) the high oxalate tolerance strain can metabolize oxalate at a concentration of about 15 mM.

10.-12. (canceled)

13. The composition of claim 1, wherein each strain comprises a 16s RNA nucleotide sequence that is
(a) at least about 80% identical to the nucleotide sequence set forth in SEQ ID NO: 42, SEQ ID NO: 79, or SEQ ID NO: 146,
(b) at least about 90% identical to the nucleotide sequence set forth in SEQ ID NO: 42, SEQ ID NO: 79, or SEQ ID NO: 146,
(c) at least about 96% identical to the nucleotide sequence set forth in SEQ ID NO: 42, SEQ ID NO: 79, or SEQ ID NO: 146 (d) at least about 97% identical to the nucleotide sequence set forth in SEQ ID NO: 42, SEQ ID NO: 79, or SEQ ID NO: 146,
(e) at least about 98.5% identical to the nucleotide sequence set forth in SEQ ID NO: 42, SEQ ID NO: 79, or SEQ ID NO: 146, or
(f) identical to the nucleotide sequence set forth in SEQ ID NO: 42, SEQ ID NO: 79, or SEQ ID NO: 146.

14.-15. (canceled)

16. The composition of claim 1 further comprising:
a) one or more microbes metabolizing formate;
b) one or more microbes catalyzing fermentation of polysaccharides;
c) one or more microbes catalyzing fermentation of amino acids;
d) one or more microbes catalyzing the synthesis of at least one molecules selected from the group consisting of methane, acetate, sulfide, propionate, and succinate; and/or
e) microbes catalyzing i) deconjugation of conjugated bile acids to produce primary bile acids, ii) conversion of cholic acid (CA) to 7-oxocholic acid, iii) conversion of 7-oxocholic acid to 7-beta-cholic acid (7betaCA), iv) conversion of chenodeoxycholic acid (CDCA) to 7-oxochenodeoxycholic acid, and/or v) conversion of 7-oxochenodeoxycholic acid to ursodeoxycholic acid (UDCA).

17.-20. (canceled)

21. The composition of claim 1, wherein the composition comprises:
a) Consortia I or a functional equivalent thereof,
b) Consortia II or a functional equivalent thereof;
c) Consortia III or a functional equivalent thereof,
d) Consortia IV or a functional equivalent thereof;
e) Consortia V or a functional equivalent thereof,
f) Consortia VI or a functional equivalent thereof,
g) Consortia VII or a functional equivalent thereof;
h) Consortia VIII or a functional equivalent thereof;
i) Consortia IX or a functional equivalent thereof;
j) Consortia X or a functional equivalent thereof,
k) Consortia XI or a functional equivalent thereof;
l) Consortia XII or a functional equivalent thereof;
m) Consortia XIII or a functional equivalent thereof,
n) Consortia XIV or a functional equivalent thereof;
o) Consortia XV or a functional equivalent thereof,
p) Consortia XVI or a functional equivalent thereof;
q) Consortia XVII or a functional equivalent thereof;
r) Consortia XVIII or a functional equivalent thereof; or
s) Consortia XIX or a functional equivalent thereof.

22. The composition of claim 1, further comprising:
(a) a second composition comprising Clostridium citroniae, Bacteroides salyersiae, Blautia obeum, Parabacteroides merdae, Parabacteroides distasonis, Anaerostipes hadrus, Lachnospiraceae sp. FBI00033, Eubacterium eligens, Bifidobacterium dentium, Blautia wexlerae, Fusicatenibacter saccharivorans, Bacteroides nordii, Dorea formicigenerans, Dorea longicatena, Bacteroides stercorirosoris, Bifidobacterium longum, Bacteroides kribbi, Lachnospiraceae sp. FBI00071, Bacteroides thetaiotaomicron, Clostridium clostridioforme, Clostridium scindens, Roseburia hominis, Clostridium fessum, Coprococcus comes, Blautia faecis, Hungatella hathewayi, Bacteroides stercoris, Collinsella aerofaciens, Hungatella effluvii, Bifidobacterium adolescentis, Bifidobacterium catenulatum, Lactobacillus rogosae, Bacteroides faecis, Bacteroides finegoldii, Clostridiaceae sp. FBI00191, Ruminococcus faecis, Lachnoclostridium pacaense, Clostridium bolteae, Longicatena caecimuris, Eggerthella lenta, Blautia massiliensis, Bacteroides xylanisolvens, Bacteroides vulgatus, Megasphaera massiliensis, Butyricimonas faecihominis, Eisenbergiella tayi, Acidaminococcus intestini, Emergencia timonensis, Bifidobacterium pseudocatenulatum, Eubacterium hallii, Anaerofustis stercorihominis, Eubacterium ventriosum, Blautia hydrogenotrophica, Lachnospiraceae sp. FBI00290, or a functional equivalent microbial consortium,
(b) a third composition comprising Acutalibacter timonensis, Alistipes onderdonkii, Bacteroides uniformis, Eubacterium rectale, Alistipes timonensis, Bacteroides kribbi, Coprococcus eutactus, Bilophila wadsworthia, Bacteroides caccae, Alistipes shahii, Parasutterella excrementihominis, Paraprevotella clara, Sutterella wadsworthensis, Sutterella massiliensis, Porphyromonas asaccharolytica, Ruminococcus bromii, Monoglobus pectinilyticus, Ruminococcaceae sp. FBI00097, Gordonibacter pamelaeae, Bacteroides uniformis, Gordonibacter pamelaeae, Bacteroides fragilis, Phascolarctobacterium faecium, Monoglobus pectinilyticus, Clostridium aldenense, Ruthenibacterium lactatiformans, Bacteroides ovatus, Bifidobacterium bifidum, Anaerotruncus massiliensis, Clostridium aldenense, Sutterella wadsworthensis, Catabacter hongkongensis, Alistipes senegalensis, Ruminococcaceae sp. FBI00233, Alistipes shahii, Dielma fastidiosa, Eubacterium siraeum, Faecalibacterium prausnitzii, Turicibacter sanguinis, Eubacterium rectale, Bacteroides caccae, Methanobrevibacter smithii, Barnesiella intestinihominis, Alistipes onderdonkii, Methanobrevibacter smithii, or a functional equivalent thereof;
(c) a fourth composition comprising Bifidobacterium adolescentis, Bifidobacterium longum, Bifidobacterium pseudocatenulatum, Bacteroides thetaiotaomicron, Coprococcus comes, Fusicatenibacter saccharivorans, Eggerthella lenta, Eubacterium eligens, Bacteroides xylanisolvens, Lactobacillus rogosae, Clostridium citroniae, Collinsella aerofaciens, Blautia obeum, Eggerthella lenta, Blautia wexlerae, Lachnoclostridium pacaense, Bacteroides vulgatus, Parabacteroides merdae, Dorea formicigenerans, Ruminococcus faecis, Roseburia hominis, Anaerostipes hadrus, Bifidobacterium adolescentis, Bifidobacterium pseudocatenulatum, Clostridium bolteae, Eisenbergiella tayi, Dorea longicatena, Eggerthella lenta, Bacteroides stercoris, Hungatella hathewayi, Bacteroides xylanisolvens, or a functional equivalent thereof; and/or
(d) a fifth composition comprising Alistipes putredinis, Dialister succinatiphilus, Akkermansia muciniphila, Ruminococcus bromii, Dialister invisus, Bacteroides massiliensis, Bilophila wadsworthia, Holdemanella biformis, Parasutterella excrementihominis, Alistipes sp. FBI00180, Bacteroides coprocola, Alistipes sp. FBI00238, Alistipes putredinis, Eubacterium xylanophilum, Senegalimassilia anaerobia, or a functional equivalent thereof.

23. The composition of claim 1, further comprising:
(a) FBI00001, FBI00002, FBI00010, FBI00013, FBI00029, FBI00032, FBI00033, FBI00034, FBI00043, FBI00044, FBI00048, FBI00050, FBI00051, FBI00057, FBI00059, FBI00060, FBI00070, FBI00071, FBI00076, FBI00079, FBI00087, FBI00093, FBI00102, FBI00109, FBI00117, FBI00120, FBI00125, FBI00127, FBI00128, FBI00145, FBI00162, FBI00174, FBI00184, FBI00190, FBI00191, FBI00194, FBI00198, FBI00199, FBI00200, FBI00201, FBI00205, FBI00206, FBI00211, FBI00220, FBI00221, FBI00236, FBI00245, FBI00248, FBI00251, FBI00254, FBI00267, FBI00278, FBI00288, FBI00290, or a functional equivalent thereof;
(b) FBI00004, FBI00012, FBI00015, FBI00018, FBI00019, FBI00021, FBI00038, FBI00040, FBI00046, FBI00061, FBI00066, FBI00075, FBI00077, FBI00080, FBI00081, FBI00085, FBI00092, FBI00097, FBI00099, FBI00112, FBI00132, FBI00137, FBI00140, FBI00149, FBI00151, FBI00176, FBI00189, FBI00197, FBI00208, FBI00212, FBI00224, FBI00226, FBI00229, FBI00233, FBI00235, FBI00237, FBI00243, FBI00244, FBI00258, FBI00260, FBI00263, FBI00270, FBI00273, FBI00277, FBI00292, or a functional equivalent thereof;
(c) FBI00009, FBI00011, FBI00016, FBI00020, FBI00025, FBI00027, FBI00030, FBI00047, FBI00052, FBI00053, FBI00056, FBI00062, FBI00078, FBI00096, FBI00104, FBI00110, FBI00111, FBI00113, FBI00115, FBI00116, FBI00123, FBI00124, FBI00126, FBI00135, FBI00147, FBI00159, FBI00167, FBI00170, FBI00232, FBI00255, FBI00271, or a functional equivalent thereof; and/or
(d) FBI00022, FBI00049, FBI00068, FBI00069, FBI00152, FBI00165, FBI00171, FBI00175, FBI00177, FBI00180, FBI00182, FBI00238, FBI00269, FBI00274, FBI00281, or a functional equivalent thereof.

24. (canceled)

25. The composition of claim 22, wherein
(a) each strain comprises a 16s RNA nucleotide sequence that is at least about 97% identical or 98.5% identical to the nucleotide sequence set forth in SEQ ID NO: 1, SEQ ID NO: 2, SEQ ID NO: 5, SEQ ID NO: 8, SEQ ID NO: 18, SEQ ID NO: 20, SEQ ID NO: 21, SEQ ID NO: 22, SEQ ID NO: 25, SEQ ID NO: 26, SEQ ID NO: 29, SEQ ID NO: 31, SEQ ID NO: 32, SEQ ID NO: 36, SEQ ID NO: 37, SEQ ID NO: 38, SEQ ID NO: 45, SEQ ID NO: 46, SEQ ID NO: 48, SEQ ID NO: 51, SEQ ID NO: 55, SEQ ID NO: 57, SEQ ID NO: 61, SEQ ID NO: 63, SEQ ID NO: 70, SEQ ID NO: 71, SEQ ID NO: 74, SEQ ID NO: 76, SEQ ID NO: 77, SEQ ID NO: 83, SEQ ID NO: 89, SEQ ID NO: 94, SEQ ID NO: 100, SEQ ID NO: 102, SEQ ID NO: 103, SEQ ID NO: 104, SEQ ID NO: 106, SEQ ID NO: 107, SEQ ID NO: 108, SEQ ID NO: 109, SEQ ID NO: 110, SEQ ID NO: 111, SEQ ID NO: 113, SEQ ID NO: 115, SEQ ID NO: 116, SEQ ID NO: 123, SEQ ID NO: 128, SEQ ID NO: 129, SEQ ID NO: 130, SEQ ID NO: 131, SEQ ID NO: 136, SEQ ID NO: 143, SEQ ID NO: 145, or SEQ ID NO: 147;
(b) each strain comprises a 16s RNA nucleotide sequence that is at least about 97% identical or 98.5% identical to the nucleotide sequence set forth in SEQ ID NO: 3, SEQ ID NO: 7, SEQ ID NO: 9, SEQ ID NO: 11, SEQ ID NO: 12, SEQ ID NO: 14, SEQ ID NO: 23, SEQ ID NO: 24, SEQ ID NO: 27, SEQ ID NO: 39, SEQ ID NO: 41, SEQ ID NO: 47, SEQ ID NO: 49, SEQ ID NO: 50, SEQ ID NO: 51, SEQ ID NO: 54, SEQ ID NO: 56, SEQ ID NO: 59, SEQ ID NO: 60, SEQ ID NO: 66, SEQ ID NO: 78, SEQ ID NO: 81, SEQ ID NO: 82, SEQ ID NO: 85, SEQ ID NO: 86, SEQ ID NO: 96, SEQ ID NO: 101, SEQ ID NO: 105, SEQ ID NO: 112, SEQ ID NO: 114, SEQ ID NO: 117, SEQ ID NO: 118, SEQ ID NO: 119, SEQ ID NO: 121, SEQ ID NO: 122, SEQ ID NO: 124, SEQ ID NO: 126, SEQ ID NO: 127, SEQ ID NO: 133, SEQ ID NO: 134, SEQ ID NO: 135, SEQ ID NO: 138, SEQ ID NO: 140, SEQ ID NO: 142, or SEQ ID NO: 148;
(c) each strain comprises a 16s RNA nucleotide sequence that is at least about 97% identical or 98.5% identical to the nucleotide sequence set forth in SEQ ID NO: 4, SEQ ID NO: 6, SEQ ID NO: 10, SEQ ID NO: 13, SEQ ID NO: 16, SEQ ID NO: 17, SEQ ID NO: 19, SEQ ID NO: 28, SEQ ID NO: 33, SEQ ID NO: 34, SEQ ID NO: 35, SEQ ID NO: 40, SEQ ID NO: 50, SEQ ID NO: 58, SEQ ID NO: 62, SEQ ID NO: 64, SEQ ID NO: 65, SEQ ID NO: 67, SEQ ID NO: 68, SEQ ID NO: 69, SEQ ID NO: 72, SEQ ID NO: 73, SEQ ID NO: 75, SEQ ID NO: 80, SEQ ID NO: 84, SEQ ID NO: 88, SEQ ID NO: 91, SEQ ID NO: 92, SEQ ID NO: 120, SEQ ID NO: 132, or SEQ ID NO: 139; and/or
(d) each strain comprises a 16s RNA nucleotide sequence that is at least about 97% identical or 98.5% identical to the nucleotide sequence set forth in SEQ ID NO: 15, SEQ ID NO: 30, SEQ ID NO: 43, SEQ ID NO: 44, SEQ ID NO: 87, SEQ ID NO: 90, SEQ ID NO: 93, SEQ ID NO: 95, SEQ ID NO: 97, SEQ ID NO: 98, SEQ ID NO: 99, SEQ ID NO: 125, SEQ ID NO: 137, SEQ ID NO: 141, or SEQ ID NO: 144.

26. The composition of claim 23, wherein
(a) each strain comprises a 16s RNA nucleotide sequence identical to the nucleotide sequence set forth in SEQ ID NO: SEQ ID NO: 1, SEQ ID NO: 2, SEQ ID NO: 5, SEQ ID NO: 8, SEQ ID NO: 18, SEQ ID NO: 20, SEQ ID NO: 21, SEQ ID NO: 22, SEQ ID NO: 25, SEQ ID NO: 26, SEQ ID NO: 29, SEQ ID NO: 31, SEQ ID NO: 32, SEQ ID NO: 36, SEQ ID NO: 37, SEQ ID NO: 38, SEQ ID NO: 45, SEQ ID NO: 46, SEQ ID NO: 48, SEQ ID NO: 51, SEQ ID NO: 55, SEQ ID NO: 57, SEQ ID NO: 61, SEQ ID NO: 63, SEQ ID NO: 70, SEQ ID NO: 71, SEQ ID NO: 74, SEQ ID NO: 76, SEQ ID NO: 77, SEQ ID NO: 83, SEQ ID NO: 89, SEQ ID NO: 94, SEQ ID NO: 100, SEQ ID NO: 102, SEQ ID NO: 103, SEQ ID NO: 104, SEQ ID NO: 106, SEQ ID NO: 107, SEQ ID NO: 108, SEQ ID NO: 109, SEQ ID NO: 110, SEQ ID NO: 111, SEQ ID NO: 113, SEQ ID NO: 115, SEQ ID NO: 116, SEQ ID NO: 123, SEQ ID NO: 128, SEQ ID NO: 129, SEQ ID NO: 130, SEQ ID NO: 131, SEQ ID NO: 136, SEQ ID NO: 143, SEQ ID NO: 145, or SEQ ID NO: 147;
(b) each strain comprises a 16s RNA nucleotide sequence identical to the nucleotide sequence set forth in SEQ ID NO: SEQ ID NO: 3, SEQ ID NO: 7, SEQ ID NO: 9, SEQ ID NO: 11, SEQ ID NO: 12, SEQ ID NO: 14, SEQ ID NO: 23, SEQ ID NO: 24, SEQ ID NO: 27, SEQ ID NO: 39, SEQ ID NO: 41, SEQ ID NO: 47, SEQ ID NO: 49, SEQ ID NO: 50, SEQ ID NO: 51, SEQ ID NO: 54, SEQ ID NO: 56, SEQ ID NO: 59, SEQ ID NO: 60, SEQ ID NO: 66, SEQ ID NO: 78, SEQ ID NO: 81, SEQ ID NO: 82, SEQ ID NO: 85, SEQ ID NO: 86, SEQ ID NO: 96, SEQ ID NO: 101, SEQ ID NO: 105, SEQ ID NO: 112, SEQ ID NO: 114, SEQ ID NO: 117, SEQ ID NO: 118, SEQ ID NO: 119, SEQ ID NO: 121, SEQ ID NO: 122, SEQ ID NO: 124, SEQ ID NO: 126, SEQ ID NO: 127, SEQ ID NO: 133, SEQ ID NO: 134, SEQ ID NO: 135, SEQ ID NO: 138, SEQ ID NO: 140, SEQ ID NO: 142, or SEQ ID NO: 148;
(c) each strain comprises a 16s RNA nucleotide sequence identical to the nucleotide sequence set forth in SEQ ID NO: SEQ ID NO: 4, SEQ ID NO: 6, SEQ ID NO: 10, SEQ ID NO: 13, SEQ ID NO: 16, SEQ ID NO: 17, SEQ ID NO: 19, SEQ ID NO: 28, SEQ ID NO: 33, SEQ ID NO: 34, SEQ ID NO: 35, SEQ ID NO: 40, SEQ ID NO: 50, SEQ ID NO: 58, SEQ ID NO: 62, SEQ ID NO: 64, SEQ ID NO: 65, SEQ ID NO: 67, SEQ ID NO: 68, SEQ ID NO: 69, SEQ ID NO: 72, SEQ ID NO: 73, SEQ ID NO: 75, SEQ ID NO: 80, SEQ ID NO: 84, SEQ ID NO: 88, SEQ ID NO: 91, SEQ ID NO: 92, SEQ ID NO: 120, SEQ ID NO: 132, or SEQ ID NO: 139; and/or
(d) each strain comprises a 16s RNA nucleotide sequence identical to the nucleotide sequence set forth in SEQ ID NO: SEQ ID NO: 15, SEQ ID NO: 30, SEQ ID NO: 43, SEQ ID NO: 44, SEQ ID NO: 87, SEQ ID NO: 90, SEQ ID NO: 93, SEQ ID NO: 95, SEQ ID NO: 97, SEQ ID NO: 98, SEQ ID NO: 99, SEQ ID NO: 125, SEQ ID NO: 137, SEQ ID NO: 141, or SEQ ID NO: 144.

27.-41. (canceled)

42. A microbial consortium comprising microbial strains set forth in Table 1, Table 2, Table 3, Table 4, Table 5, Table 6, Table 7, Table 8, Table 9, Table 10, Table 11, Table 12, Table 13, Table 14, Table 15, Table 16, Table 17, Table 18, Table 19, Table 22, or a functional equivalent thereof.

43.-46. (canceled)

47. A composition comprising a microbial consortium of claim 42.

48. (canceled)

49. The microbial consortium of claim 42, comprising from about 5×10¹⁰ to about 5×10¹¹ viable cells, from about 5×10⁹ to about 5×10¹⁰ viable cells, from about 5×10¹¹ to about 5×10¹¹ viable cells, or up to about 5×1012 viable cells.

50.-53. (canceled)

54. The composition of claim 1, wherein the composition comprises from about 10% to about 50% of O. formigenes strains on a viable cell count basis, about 20% of O. formigenes strains on a viable cell count basis, about 30% of O. formigenes strains on a viable cell count basis, or about 40% of O. formigenes strains on a viable cell count basis.

55.-57. (canceled)

58. A method of manufacturing the composition of claim 1, the method comprising
1) obtaining and blending:
a) a first composition comprising Clostridium citroniae, Bacteroides salyersiae, Blautia obeum, Parabacteroides merdae, Parabacteroides distasonis, Anaerostipes hadrus, Lachnospiraceae sp. FBI00033, Eubacterium eligens, Bifidobacterium dentium, Blautia wexlerae, Fusicatenibacter saccharivorans, Bacteroides nordii, Dorea formicigenerans, Dorea longicatena, Bacteroides stercorirosoris, Bifidobacterium longum, Bacteroides kribbi, Lachnospiraceae sp. FBI00071, Bacteroides thetaiotaomicron, Clostridium clostridioforme, Clostridium scindens, Roseburia hominis, Clostridium fessum, Coprococcus comes, Blautia faecis, Hungatella hathewayi, Bacteroides stercoris, Collinsella aerofaciens, Hungatella effluvii, Bifidobacterium adolescentis, Bifidobacterium catenulatum, Lactobacillus rogosae, Bacteroides faecis, Bacteroides finegoldii, Clostridiaceae sp. FBI00191, Ruminococcus faecis, Lachnoclostridium pacaense, Clostridium bolteae, Longicatena caecimuris, Eggerthella lenta, Blautia massiliensis, Bacteroides xylanisolvens, Bacteroides vulgatus, Megasphaera massiliensis, Butyricimonas faecihominis, Eisenbergiella tayi, Acidaminococcus intestini, Emergencia timonensis, Bifidobacterium pseudocatenulatum, Eubacterium hallii, Anaerofustis stercorihominis, Eubacterium ventriosum, Blautia hydrogenotrophica, and Lachnospiraceae sp. FBI00290, or a functional equivalent thereof,
b) a second composition comprising Acutalibacter timonensis, Alistipes onderdonkii, Bacteroides uniformis, Eubacterium rectale, Alistipes timonensis, Bacteroides kribbi, Coprococcus eutactus, Bilophila wadsworthia, Bacteroides caccae, Alistipes shahii, Parasutterella excrementihominis, Paraprevotella clara, Sutterella wadsworthensis, Sutterella massiliensis, Porphyromonas asaccharolytica, Ruminococcus bromii, Monoglobus pectinilyticus, Ruminococcaceae sp. FBI00097, Gordonibacter pamelaeae, Bacteroides uniformis, Gordonibacter pamelaeae, Bacteroides fragilis, Phascolarctobacterium faecium, Monoglobus pectinilyticus, Clostridium aldenense, Ruthenibacterium lactatiformans, Bacteroides ovatus, Bifidobacterium bifidum, Anaerotruncus massiliensis, Clostridium aldenense, Sutterella wadsworthensis, Catabacter hongkongensis, Alistipes senegalensis, Ruminococcaceae sp. FBI00233, Alistipes shahii, Dielma fastidiosa, Eubacterium siraeum, Faecalibacterium prausnitzii, Turicibacter sanguinis, Eubacterium rectale, Bacteroides caccae, Methanobrevibacter smithii, Barnesiella intestinihominis, Alistipes onderdonkii, and Methanobrevibacter smithii, or a functional equivalent thereof;
c) a third composition comprising Bifidobacterium adolescentis, Bifidobacterium longum, Bifidobacterium pseudocatenulatum, Bacteroides thetaiotaomicron, Coprococcus comes, Fusicatenibacter saccharivorans, Eggerthella lenta, Eubacterium eligens, Bacteroides xylanisolvens, Lactobacillus rogosae, Clostridium citroniae, Collinsella aerofaciens, Blautia obeum, Eggerthella lenta, Blautia wexlerae, Lachnoclostridium pacaense, Bacteroides vulgatus, Parabacteroides merdae, Dorea formicigenerans, Ruminococcus faecis, Roseburia hominis, Anaerostipes hadrus, Bifidobacterium adolescentis, Bifidobacterium pseudocatenulatum, Clostridium bolteae, Eisenbergiella tayi, Dorea longicatena, Eggerthella lenta, Bacteroides stercoris, Hungatella hathewayi, and Bacteroides xylanisolvens, or a functional equivalent thereof;
d) a fourth composition comprising Alistipes putredinis, Dialister succinatiphilus, Akkermansia muciniphila, Ruminococcus bromii, Dialister invisus, Bacteroides massiliensis, Bilophila wadsworthia, Holdemanella biformis, Parasutterella excrementihominis, Alistipes sp. FBI00180, Bacteroides coprocola, Alistipes sp. FBI00238, Alistipes putredinis, Eubacterium xylanophilum, and Senegalimassilia anaerobia, or a functional equivalent thereof;
e) a fifth composition comprising a first O. formigenes strain;
f) a sixth composition comprising a second O. formigenes strain; and/or
g) a seventh composition comprising a third O. formigenes strain; or
2) obtaining and blending:
a) a first composition comprising FBI00001, FBI00002, FBI00010, FBI00013, FBI00029, FBI00032, FBI00033, FBI00034, FBI00043, FBI00044, FBI00048, FBI00050, FBI00051, FBI00057, FBI00059, FBI00060, FBI00070, FBI00071, FBI00076, FBI00079, FBI00087, FBI00093, FBI00102, FBI00109, FBI00117, FBI00120, FBI00125, FBI00127, FBI00128, FBI00145, FBI00162, FBI00174, FBI00184, FBI00190, FBI00191, FBI00194, FBI00198, FBI00199, FBI00200, FBI00201, FBI00205, FBI00206, FBI00211, FBI00220, FBI00221, FBI00236, FBI00245, FBI00248, FBI00251, FBI00254, FBI00267, FBI00278, FBI00288, and FBI00290, or a functional equivalent thereof;
b) a second composition comprising FBI00004, FBI00012, FBI00015, FBI00018, FBI00019, FBI00021, FBI00038, FBI00040, FBI00046, FBI00061, FBI00066, FBI00075, FBI00077, FBI00080, FBI00081, FBI00085, FBI00092, FBI00097, FBI00099, FBI00112, FBI00132, FBI00137, FBI00140, FBI00149, FBI00151, FBI00176, FBI00189, FBI00197, FBI00208, FBI00212, FBI00224, FBI00226, FBI00229, FBI00233, FBI00235, FBI00237, FBI00243, FBI00244, FBI00258, FBI00260, FBI00263, FBI00270, FBI00273, FBI00277, and FBI00292, or a functional equivalent thereof;
c) a third composition comprising FBI00009, FBI00011, FBI00016, FBI00020, FBI00025, FBI00027, FBI00030, FBI00047, FBI00052, FBI00053, FBI00056, FBI00062, FBI00078, FBI00096, FBI00104, FBI00110, FBI00111, FBI00113, FBI00115, FBI00116, FBI00123, FBI00124, FBI00126, FBI00135, FBI00147, FBI00159, FBI00167, FBI00170, FBI00232, FBI00255, and FBI00271, or a functional equivalent thereof;
d) a fourth composition comprising FBI00022, FBI00049, FBI00068, FBI00069, FBI00152, FBI00165, FBI00171, FBI00175, FBI00177, FBI00180, FBI00182, FBI00238, FBI00269, FBI00274, and FBI00281, or a functional equivalent thereof;
e) a fifth composition comprising FBI00067 or a functional equivalent thereof;
f) a sixth composition comprising FBI00133 or a functional equivalent thereof; and/or
g) a seventh composition comprising FBI00289 or a functional equivalent thereof.

59.-62. (canceled)

63. The method of claim 58, wherein
(a) the fourth composition is obtained by growing microbes in presence of threonine;
(b) each composition comprises a lyoprotectant;
(c) each composition comprises maltodextrin, inulin, or a combination thereof; and/or
(d) each composition is separately lyophilized.

64.-72. (canceled)

73. The method of claim 58, wherein the functional equivalent is based on the characteristics set forth in Tables 24 or 34-36.

74. The method of any one of claims 58-73 comprising obtaining and blending
(a) microbes comprising a gene regulating oxalate degradation, oxalate resistance, formate metabolism, metabolism of macronutrients, production of microbial metabolites, cross-feeding activity, and/or mucin degradation,
(b) microbes that are known to protect against diseases and/or that are prevalent in healthy human gut;
(c) microbes that utilize carbon sources set forth in Table 35.

75.-77. (canceled)

78. The method of claim 58, wherein
(a) each composition is prepared using inoculation density adjustment;
(b) each composition is cultured or has been cultured in presence of gas overlay;
(c) each composition is cultured or has been cultured in absence of gas sparging.

79.-80. (canceled)

81. A composition prepared by the method of claim 58.

82. A method of treating hyperoxaluria, reducing the risk of developing hyperoxaluria, and/or reducing urinary oxalate in a subject in need thereof comprising administering an effective amount of the composition of claim 1.

83.-84. (canceled)

85. The method of claim 82, wherein the hyperoxaluria is a primary hyperoxaluria, a secondary hyperoxaluria, or an enteric hyperoxaluria.

86.-87. (canceled)

88. The method of claim 82, further comprising administering at least one antibacterial agent, antiviral agent, antifungal agent, anti-inflammatory agent, immunosuppressive agent, prebiotic, a low oxalate diet, a high hydration diet, calcium supplements, or a combination thereof.

89. The method of claim 82, further comprising administering NOV-001, SYNB8802, OX-1, Lumasiran, Nedosiran, BBP-711, CNK-336, PBGENE-PH1, or a combination thereof.

90.-91. (canceled)

92. A method of treating hyperoxaluria, reducing the risk of developing hyperoxaluria, and/or reducing urinary oxalate in a subject in need thereof comprising administering a first dose and two or more additional doses of the composition of claim 1.

93.-94. (canceled)

95. The method of claim 92, wherein the hyperoxaluria is a primary hyperoxaluria, a secondary hyperoxaluria, or an enteric hyperoxaluria.

96.-97. (canceled)

98. The method of claim 92, further comprising administering an antibiotic treatment.

99.-100. (canceled)

101. The method of claim 98, wherein the antibiotic treatment is completed 1 day or 2 days before administering the first dose.

102.-116. (canceled)

117. A kit comprising the composition of claim 1.

118.-121. (canceled)

122. A method of culturing a microbial strain from the Akkermansia genus comprising contacting the strain with N-Acetylgalactosamine (GalNAc).

123. The method of claim 122, wherein the strain is Akkermansia muciniphilia.

124.-130. (canceled)


SEQUENCE LISTING (148 sequences)

SEQ ID NO: 1 - DNA, 1393 bp, synthetic construct
  misc_feature 1..1393: note = Synthetic: FBI00001
  source 1..1393: mol_type = other DNA
gcagtcgnacgaagcaatcagaatgaanttttcggatggatttctggttgactgagtggcggacgggtgagtaacgcgtggataacctgcctcacactgggggataacagttagaaatggctgctaataccgcataagcgcacagtaccgcatggtacggtgtgaaaaactccggtggtgtgagatggatccgcgtctgattagttagttggcggggtaacggcccaccaagacagcgatcagtagccgacctgagagggtgaccggccacattgggactgagacacggcccaaactcctacgggaggcagcagtggggaatattgcacaatgggcgaaagcctgatgcagcgacgccgcgtgagtgaagaagtatttcggtatgtaaagctctatcagcagggaagaaaatgacggtacctgactaagaagccccggctaactacgtgccagcagccgcggtaatacgtagggggcaagcgttatccggatttactgggtgtaaagggagcgtagacggcgaagcaagtctggagtgaaaacccagggctcaaccctgggactgctttggaaactgttttgctagagtgtcggagaggtaagtggaattcctagtgtagcggtgaaatgcgtagatattaggaggaacaccagtggcgaaggcggcttactggacgataactgacgttgaggctcgaaagcgtggggagcaaacaggattagataccctggtagtccacgccgtaaacgatgaatgctaggtgttggggggcaaagcccttcggtgccgtcgcaaacgcaataagcattccacctggggagtacgttcgcaagaatgaaactcaaaggaattgacggggacccgcacaagcggtggagcatgtggtttaattcgaagcaacgcgaagaaccttaccaagtcttgacatccccctgaccggtcagtaaagtgacctttccttcgggacaggggagacaggtggtgcatggttgtcgtcagctcgtgtcgtgagatgttgggttaagtcccgcaacgagcgcaacccttatccttagtagccagcaggtaaagctgggcactctagggagactgccagggataacctggaggaaggtggggatgacgtcaaatcatcatgccccttatgatttgggctacacacgtgctacaatggcgtaaacaaagggaagcgaccctgcgaaggcaagcaaatcccaaaaataacgtcccagttcggactgtagtctgcaacccgactacacgaagctggaatcgctagtaatcgcgaatcagaatgtcgcggtgaatacgttcccgggtcttgtacacaccgcccgtcacaccatgggagtcagcaacgcccgaagtcaagtgacccaaccgaaaggaggg

SEQ ID NO: 2 - DNA, 1397 bp, synthetic construct
  misc_feature 1..1397: note = Synthetic: FBI00002
  source 1..1397: mol_type = other DNA
anncntgcaagtcgaggggnagcnnggtgtagcaatacaccgcgtggcgaccggcgcacgggtgagtaacacgtatccaacctgccctttactcggggatagcctttcgaaagaaagattaatacccgatggtataacatgacctcctggttttgttattaaagaatttcggtagaggatggggatgcgttccattaggcagttggcggggtaacggcccaccaaaccttcgatggataggggttctgagaggaaggtcccccacattggaactgagacacggtccaaactcctacgggaggcagcagtgaggaatattggtcaatgggcgagagcctgaaccagccaagtagcgtgaaggatgaccgccctatgggttgtaaacttcttttatatgggaataaagggtgccacgtgtggcattttgtatgtaccatatgaataaggatcggctaactccgtgccagcagccgcggtaatacggaggatccgagcgttatccggatttattgggtttaaagggagcgtaggtggacatgtaagtcagttgtgaaagtttgcggctcaaccgtaaaattgcagttgatactgcgtgtcttgagtacagtagaggtgggcggaattcgtggtgtagcggtgaaatgcttagatatcacgaagaactccgattgcgaaggcagctcactggactgcaactgacactgatgctcgaaagtgtgggtatcaaacaggattagataccctggtagtccacacagtaaacgatgaatactcgctgtttgcgatatacagtaagcggccaagcgaaagcattaagtattccacctggggagtacgccggcaacggtgaaactcaaaggaattgacgggggcccgcacaagcggaggaacatgtggtttaattcgatgatacgcgaggaaccttacccgggcttaaattgcaaatgaatatgccggaaacggcatagccgcaaggcatttgtgaaggtgctgcatggttgtcgtcagctcgtgccgtgaggtgtcggcttaagtgccataacgagcgcaacccttatcttcagttactaacaggttatgctgaggactctggagagactgccgtcgtaagatgtgaggaaggtggggatgacgtcaaatcagcacggcccttacgtccggggctacacacgtgttacaatggggggtacagaaggccgctacacagcgatgtgatgccaatccctaaagcccctctcagttcggatcgaagtctgcaacccgacttcgtgaagctggattcgctagtaatcgcgcatcagccacggcgcggtgaatacgttcccgggccttgtacacaccgcccgtcaagccatgggagccgggggtacnngaagtacgtaaccgcaaggagcnnc

SEQ ID NO: 3 - DNA, 1404 bp, synthetic construct
  misc_feature 1..1404: note = Synthetic: FBI00004
  source 1..1404: mol_type = other DNA
tannnntgcagtcgaacggagatagacgctgaaagggagacagcttgctgtaagaatttcttgtttatcttagtggcggacgggtgagtaacgcgtgagtaacctgcctttcagagggggataacgtctggaaacggacgctaataccgcatgagaccacagcttcacatggagcggcggtcaaaggagcaatccgctgaaagatggactcgcgtccgattagatagttggcggggtaacggcccaccaagtcgacgatcggtagccggactgagaggttgaacggccacattgggactgagacacggcccagactcctacgggaggcagcagtgagggatattggtcaatgggggaaaccctgaaccagcaacgccgcgtgagggaagacggttttcggattgtaaacctctgtcctctgtgaagatagtgacggtagcagaggaggaagctccggctaactacgtgccagcagccgcggtaatacgtagggagcgagcgttgtccggatttactgggtgtaaagggtgcgtaggcggctctgcaagtcagaagtgaaatccatgggcttaacccatgaactgcttttgaaaactgtagagcttgagtgaagtagaggtaggcggaattcccggtgtagcggtgaaatgcgtagagatcgggaggaacaccagtgggcgaanggcgnctactgggctttaactgacgctgagcacgaaagcatgggtagcaaacaggattagataccctggtagtccatgccgtaaacgatgattactaggntgtgggggggtctgaccccctccgtgccggagttaacacaataagtaatccaccnggggagtacgaccgcaaggttgaaactcaaaggaattgacgggggcccgcacaagcagtggagtatgtggattaattngaagcaacgcgaaganccttaccaggtcttgacatccaactaacgaagcagagatgcattaggtgcccttcggggaaagttgagacaggtggtgcatggttgtcgtcagctcgtgtcgtgagatgttgggttaagtcccgcaacgagcgcaacccttactgttagttgctacgcaagagcactctagcaggactgccgttgacaaaacggaggaaggtggggacgacgtcaaatcatcatgccccttatgacctgggcctcacacgtactacaatggccattaacagagggaagcaagcccgcgaggtggagcaaaaccctaaaaatggtctcagttcggatcgtaggctgaaacccgcctgcgtgaagttggaattgctagtaatcgcggatcagcatgccgcggtgaatacgttcccgggccttgtacacaccgcccgtcacaccatgggagccggtaatacccgaagtcagtagtctaaccgcaa

SEQ ID NO: 4 - DNA, 1285 bp, synthetic construct
  misc_feature 1..1285: note = Synthetic: FBI00009
  source 1..1285: mol_type = other DNA
tcgacgggatcccaggagcttgctcctgggtgagagtggcgaacgggtgagtaatgcgtgaccgacctgccccatacaccggaatagctcctggaaacgggtggtaatgccggatgctccagttgaccgcatggtcttctgggaaagcttttgcggtatgggatggggtcgcgtcctatcagcttgatggcggggtaacggcccaccatggcttcgacgggtagccggcctgagagggcgaccggccacattgggactgagatacggcccanactcctacgggaggcagcagtggggaatattgcacaatgggcgcaagcctgatgcagcgacgccgcgtgcgggatgacggccttcgggttgtaaaccgcttttgactgggagcaagcccttcggggtgagtgtacctttcgaataancaccggctaactacgtgccagcagccgcggtaatacgtanggtgcaagcgttatccggaattattgggcgtaaagggctcgtangcggttcgtcgcgtccggtgtgaaagtccatcgcttaacggtggatccgcgccnggtacgggcgggcttgagtgcggtaggggagactggaattccccggtgtaacggtggaatgtgtagatatcgggaagaacaccaatggcgaaggcaggtctctgggccgtcactgacgctgaggagcgaaagcgtggggagcgaacaggattagataccctggtagtccacgccgtaaacggtggatgctggatgtggggaccattccacggtctccgtgtcggagccaacgcgttaagcatcccgcctggggagtacggccgcaaggctaaaactcaaagaaatgacgggggcccgcacaagcggcggagcatgcggattaattcgatgcaacgcgaagaaccttacctgggcttgacatgttcccgacagccgtagagatacggtctcccttcggggcgggttcacaggtggtgcatggtcgtcgtcagctcgtgtcgtgagatgttgggttaagtcccgcaacgagcgcaaccctcgccctgtgttgccagcacgtcgtggtgggaactcacgggggaccgccggggtcaactcggaggaagggggggaagacgtcagatcatcatgccccttacgtccagggcttcacgcatgctacaatggccggtacaacgggatgcgacaccgcgaggtggagcgaatcccttaaaaccggtctcagttcggattggagtctgcaaccggactccatgaaggcggagtcgntagtaatggcggatcagcaa

SEQ ID NO: 5 - DNA, 1390 bp, synthetic construct
  misc_feature 1..1390: note = Synthetic: FBI00010
  source 1..1390: mol_type = other DNA
tgcagtcgaacgggaacctttnattgaagcttcggcagatttagctggtttctagtggcggacgggtgagtaacgcgtgggtaacctgccttatacagggggataacaaccagaaatggttgctaataccgcataagcgcacaggaccgcatggtccggtgtgaaaaactccggtggtataagatggacccgcgttggattagctagttggcagggtaacggcctaccaaggcgacgatccatagccggcctgagagggtgaacggccacattgggactgaaacacggcccagactcctacgggaggcagcagtggggaatattgcacaatgggggaaaccctgatgcagcgacgccgcgtgaaggaagaagtatctcggtatgtaaacttctatcagcagggaagatagtgacggtacctgactaagaagccccggctaactacgtgccagcagccgcggtaatacgtagggggcaagcgttatccggatttactgggtgtaaagggagcgtagacggactggcaagtctgatgtgaaaggcgggggctcaacccctggactgcattggaaactgttagtcttgagtgccggagaggtaagcggaattcctagtgtagcggtgaaatgcgtagatattaggaggaacaccagtggcgaaggcggcttactggacggtaactgacgttgaggctcgaaagcgtggggagcaaacaggattagataccctggtagtccacgccgtaaacgatgaatactaggtgttggggagcaaagctcttcggtgccgccgcaaacgcattaagtattccacctggggagtacgttcgcaagaatgaaactcaaaggaattgacggggacccgcacaagcggtggagcatgtggtttaattcgaagcaacgcgaagaaccttaccaagtcttgacatccctctgaccgnncntaaccgagactttccttcgggacaggggagacaggtggtgcatggttgtcgtcagctcgtgtcgtgagatgttgggttaagtcccgcaacgagcgcaacccctatccccagtagccagcggtccggccgggcactctgaggagactgccagggataacctggaggaaggcggggatgacgtcaaatcatcatgccccttatgatttgggctacacacgtgctacaatggcgtaaacaaagggaagcaagcctgcgaaggtaagcaaatcccaaaaataacgtcccagttcggactgcagtctgcaactcgactgcacgaagctggaatcgctagtaatcgcggatcagaatgccgcggtgaatacgttcccgggtcttgtacacaccgcccgtcacaccatgggagtcagtaacgcccgaagtcagtgacctaactgcaaagaagga

SEQ ID NO: 6 - DNA, 1383 bp, synthetic construct
  misc_feature 1..1383: note = Synthetic: FBI00011
  source 1..1383: mol_type = other DNA
tgcagtcgnncgggatccatcaggctttgcttggtggtgagagtggcgaacgggtgagtaatgcgtgaccgacctgccccatacaccggaatagctcctggaaacgggtggtaatgccggatgctccagttgatcgcatggtcttctgggaaagctttcgcggtatgggatggggtcgcgtcctatcagcttgacggcggggtaacggcccaccgtggcttcgacgggtagccggcctgagagggcgaccggccacattgggactgagatacggcccagactcctacgggaggcagcagtggggaatattgcacaatgggcgcaagcctgatgcagcgacgccgcgtgagggatggaggccttcgggttgtaaacctcttttatcggggagcaagcgagagtgagtttacccgttgaataagcaccggctaactacgtgccagcagccgcggtaatacgtagggtgcaagcgttatccggaattattgggcgtaaagggctcgtaggcggttcgtcgcgtccggtgtgaaagtccatcgcttaacggtggatccgcgccgggtacgggcgggcttgagtgcggtaggggagactggaattcccggtgtaacggtggaatgtgtagatatcgggaagaacaccaatggcgaaggcaggtctctgggccgttactgacgctgaggagcgaaagcgtggggagcgaacaggattagataccctggtagtccacgccgtaaacggtggatgctggatgtggggcccgttccacgggttccgtgtcggagctaacgcgttaagcatcccgcctggggagtacggccgcaaggctaaaactcaaagaaattgacgggggcccgcacaagcggcggagcatgcggattaattcgatgcaacgcgaagaaccttacctgggcttgacatgttcccgacggtcgtagagatacggcttcccttcggggcgggttcacaggtggtgcatggtcgtcgtcagctcgtgtcgtgagatgttgggttaagtcccgcaacgagcgcaaccctcgccccgtgttgccagcggattatgccgggaactcacgggggaccgccggggttaactcggaggaaggtggggatgacgtcagatcatcatgccccttacgtccagggcttcacgcatgctacaatggccggtacaacgggatgcgacgcggcgacgcggagcggatccctgaaaaccggtctcagttcggatcgcagtctgcaactcgactgcgtgaaggcggagtcgctagtaatcgcgaatcagcaacgtcgcggtgaatgcgttcccgggccttgtacacaccgcccgtcaagtcatgaaagtgggcagcacccgaagccggtggcctaacccctttgtgg

SEQ ID NO: 7 - DNA, 1400 bp, synthetic construct
  misc_feature 1..1400: note = Synthetic: FBI00012
  source 1..1400: mol_type = other DNA
anncntgcagtcgaggggcatcgggattgaagcttgcttcaattgccggcgaccggcgcacgggtgcgtaacgcgtatgtaacctacctataacaggggcataacactgagaaattggtactaattccccataatattcggagaggcatctctccgggttgaaaactccggtggttatagatggacatgcgttgtattagctagttggtgaggtaacggctcaccaaggcaacgatacatagggggactgagaggttaaccccccacactggtactgagacacggaccagactcctacgggaggcagcagtgaggaatattggtcaatggacgcaagtctgaaccagccatgccgcgtgcaggaagacggctctatgagttgtaaactgcttttgtacgagggtaaactcacctacgtgtaggtgactgaaagtatcgtacgaataaggatcggctaactccgtgccagcagccgcggtaatacggaggattcaagcgttatccggatttattgggtttaaagggtgcgtaggcggtttgataagttagaggtgaaatcccggggcttaactccggaactgcctctaatactgttagactagagagtagttgcggtaggcggaatgtatggtgtagcggtgaaatgcttagagatcatacagaacaccgattgcgaaggcagcttaccaaactatatctgacgttgaggcacgaaagcgtggggagcaaacaggattagataccctggtagtccacgcagtaaacgatgataactcgttgtcggcgatacacagtcggtgactaagcgaaagcgataagttatccacctggggagtacgttcgcaagaatgaaactcaaaggaattgacgggggcccgcacaagcggaggaacatgtggtttaattcgatgatacgcgaggaaccttacccgggcttgaaagttactgacgattctggaaacaggatttcccttcggggcaggaaactaggtgctgcatggttgtcgtcagctcgtgccgtgaggtgtcgggttaagtcccataacgagcgcaacccctaccgttagttgccatcaggtcaagctgggcactctggcgggactgccggtgtaagccgagaggaaggtggggatgacgtcaaatcagcacggcccttacgtccggggctacacacgtgttacaatggtaggtacagagggtcgctactccgtgaggagatgccaatctcgaaagcctatctcagttcggattggaggctgaaacccgcctccatgaagttggattcgctagtaatcgcgcatcagccatggcgcggtgaatacgttcccgggccttgtacacaccgcccgtcaagccatggaagctgggggtgcctgaagttcgtgaccgcaaggagcgnc

SEQ ID NO: 8 - DNA, 1394 bp, synthetic construct
  misc_feature 1..1394: note = Synthetic: FBI00013
  source 1..1394: mol_type = other DNA
cngtcgaggggcagcatgatttgtancnntacanantgatggcgaccggcgcacgggtgagtaacgcgtatgcaacttacctatcagagggggatagcccggcgaaagtcggattaataccccataaaacaggggtcccgcatgggaatatttgttaaagattcatcgctgatagataggcatgcgttccattaggcagttggcggggtaacggcccaccaaaccgacgatggataggggttctgagaggaaggtcccccacattggtactgagacacggaccaaactcaaacgggaggcagcagtgaggaatattggtcaatggccgagaggctgaaccagccaagtcgcgtgaaggaagaaggatctatggtttgtaaacttcttttataggggaataaagtggaggacgtgtccttttttgtatgtaccctatgaataagcatcggctaactccgtgccagcagccgcggtaatacggaggatgcgagcgttatccggatttattgggtttaaagggtgcgtaggtggtgatttaagtcagcggtgaaagtttgtggctcaaccataaaattgccgttgaaactgggttacttgagtgtgtttgaggtaggcggaatgcgtggtgtagcggtgaaatgcatagatatcacgcagaactccgattgcgaaggcagcttactaaaccataactgacactgaagcacgaaagcgtggggatcaaacaggattagataccctggtagtccacgcagtaaacgatgattactaggagtttgcgatacaatgtaagctctacagcgaaagcgttaagtaatccacctggggagtacgccggcaacggtgaaactcaaaggaattgacgggggcccgcacaagcggaggaacatgtggtttaattcgatgatacgcgaggaaccttacccgggtttgaacgtagtctgaccggagtggaaacactccttctagcaatagcagattacgaggtgctgcatggttgtcgtcagctcgtgccgtgaggtgtcggcttaagtgccataacgagcgcaacccttatcactagttactaacaggtgaagctgaggactctggtgagactgccagcgtaagctgtgaggaaggtggggatgacgtcaaatcagcacggcccttacatccggggcgacacacgtgttacaatggcatggacaaagggcagctacctggcgacaggatgctaatctccaaaccatgtctcagttcggatcggagtctgcaactcgactccgtgaagctggattcgctagtaatcgcgcatcagccatggcgcggtgaatacgttcccgggccttgtacacaccgcccgtcaagccatgggagccgggggtacctgaagtccgtaaccgcaaggatcg

SEQ ID NO: 9 - DNA, 1357 bp, synthetic construct
  misc_feature 1..1357: note = Synthetic: FBI00015
  source 1..1357: mol_type = other DNA
gctannnnnnatggcgaccggcgcaggggtgagtaacacgtatgccaacctgccgatgactcggggatagcctttcgaaagaaagattaatacccgatggcataattcttccgcatggtagaactattaaagaatttcggtcatcgatggggatgcgttccattaggttgttggcggggtaacggcccaccaagccttcgatggataggggttctgagaggaaggtcccccacattggaactgagacacggtccaaactcctacgggaggcagcagtgaggaatattggtcaatggacgagagtctgaaccagccaagtagcgtgaaggatgactgccctatgggttgtaaacttcttttatacgggaataaagtgaggcacgtgtgcctttttgtatgtaccgtatgaataaggatcggctaactccgtgccagcagccgcggtaatacggaggatccgagcgttatccggatttattgggtttaaagggagcgtaggcggacgcttaagtcagttgtgaaagtttgcggctcaaccgtaaaattgcagttgatactgggtgtcttgagtacagtagaggcaggcggaattcgtggtgtagcggtgaaatgcttagatatcacgaagaactccgattgcgaaggcagcttgctggactgtaactgacgctgatgctcgaaagtgtgggtatcaaacaggattagataccctggtagtccacacagtaaacgatgaatactcgctgtttgcgatatacagtaagcggccaagcgaaagcgttaagtattccacctggggagtacgccggcaacggtgaaactcaaaggaattgacgggggcccgcacaagcggaggaacatgtggtttaattcgatgatacgcgaggaaccttacccgggcttgaattgcaactgaatgatgtggagacatgtcagccgcaaggcagttgtgaaggtgctgcatggttgtcgtcagctcgtgccgtgaggtgtcggcttaagtgccataacgagcgcaacccttatcgatagttaccatcaggttatgctggggactctgtcgagactgccgtcgtaagatgtgaggaaggtggggatgacgtcaaatcagcacggcccttacgtccggggctacacacgtgttacaatggggggtacagaaggcagctacacggcgacgtgatgctaatccctaaagcctctctcagttcggattggagtctgcaacccgactccatgaagctggattcgctagtaatcgcgcatcagccacggcgcggtgaatacgttcccgggccttgtacacaccgcccgtcaagccatgaaagccgggggtacctgaagtgcgtaaccgcga

SEQ ID NO: 10 - DNA, 1390 bp, synthetic construct
  misc_feature 1..1390: note = Synthetic: FBI00016
  source 1..1390: mol_type = other DNA
tgcagtcgnacgggatccatcaggctttgcttggtggtgagagtggcgaacgggtgagtaatgcgtgaccgacctgccccatacaccggaatagctcctggaaacgggtggtaatgccggatgctccgactcctcgcatggggtgtcgggaaagatttcatcggtatgggatggggtcgcgtcctatcaggtagtcggcggggtaacggcccaccgagcctacgacgggtagccggcctgagagggcgaccggccacattgggactgaaatacggcccaaactcctacgggaggcagcagtggggaatattgcacaatgggcgcaagcctgatgcagcgacgccgcgtgcgggatgacggccttcgggttgtaaaccgcttttgatcgggagcaagccttcgggtgagtgtacctttcgaataagcaccggctaactacgtgccagcagccgcggtaatacgtagggtgcaagcgttatccggaattattgggcgtaaagggctcgtaggcggttcgtcgcgtccggtgtgaaagtccatcgcttaacggtggatctgcgccgggtacgggcgggctggagtgcggtaggggagactggaattcccggtgtaacggtggaatgtgtagatatcgggaagaacaccaatggcgaaggcaggtctctgggccgttactgacgctgaggagcgaaagcgtggggagcgaacaggattagataccctggtagtccacgccgtaaacggtggatgctggatgtggggcccgttccacgggttccgtgtcggagctaacgcgttaagcatcccgcctggggagtacggccgcaaggctaaaactcaaagaaattgacgggggcccgcacaagcggcggagcatgcggattaattcgatgcaacgcgaagaaccttacctgggcttgacatgttcccgacagccgtagagatatggcctcccttcggggcgggttcacaggtggtgcatggtcgtcgtcagctcgtgtcgtgagatgttgggttaagtcccgcaacgagcgcaaccctcgccctgtgttgccagcacgtcatggtgggaactcacgggggaccgccggggtcaactcggaggaaggtggggatgacgtcagatcatcatgccccttacgtccagggcttcacgcatgctacaatggccggtacaacgggatgcgacacggcgacgtggagcggatccctgaaaaccggtctcagttcggattggagtctgcaacccgactccatgaaggcggagtcgctagtaatcgcggatcagcaacgccgcggtgaatgcgttcccgggccttgtacacaccgcccgtcaagtcatgaaagtgggtagcacccgaagccggtggcctaaccctttgtggangga

SEQ ID NO: 11 - DNA, 1400 bp, synthetic construct
  misc_feature 1..1400: note = Synthetic: FBI00018
  source 1..1400: mol_type = other DNA
annnntgcagtcgaacgaagcactttatttgatttccttcgggactgattattttgtgactgagtggcggacgggtgagtaacgcgtgggtaacctgccttgtacagggggataacagttggaaacggctgctaataccgcataagcgcacagcatcgcatgatgcagtgtgaaaaactccggtggtataagatggacccgcgttggattagctagttggtgaggtaacggcccaccaaggcgacgatccatagccgacctgagagggtgaccggccacattgggactgagacacggcccaaactcctacgggaggcagcagtggggaatattgcacaatgggcgaaagcctgatgcagcgacgccgcgtgagcgaagaagtatttcggtatgtaaagctctatcagcagggaagataatgacggtacctgactaagaagcaccggctaaatacgtgccagcagccgcggtaatacgtatggtgcaagcgttatccggatttactgggtgtaaagggagcgcaggcggtgcggcaagtctgatgtgaaagcccggggctcaaccccggtactgcattggaaactgtcgtactagagtgtcggaggggtaagcggaattcctagtgtagcggtgaaatgcgtagatattaggaggaacaccagtggcgaaggcggcttactggacgataactgacgctgaggctcgaaagcgtggggagcaaacaggattagataccctggtagtccacgccgtaaacgatgaatactaggtgttgggaagcattgcttctcggtgccgtcgcaaacgcagtaagtattccacctggggagtacgttcgcaagaatgaaactcaaaggaattgacggggacccgcacaagcggtggagcatgtggtttaattcgaagcaacgcgaagaaccttaccaagtcttgacatccttctgaccggtacttaaccgtatcttctcttcggagcaggagtgacaggtggtgcatggttgtcgtcagctcgtgtcgtgagatgttgggttaagtcccgcaacgagcgcaacccttatctttagtagccagcggttcggccgggcactctagagagactgccagggataacctggaggaaggcggggatgacgtcaaatcatcatgccccttatgacttgggctacacacgtgctacaatggcgtaaacaaagggaagcaaagctgtgaagccgagcaaatctcaaaaataacgtctcagttcggactgtagtctgcaacccgactacacgaagctggaatcgctagtaatcgcagatcagaatgctgcggtgaatacgttcccgggtcttgtacacaccgcccgtcacaccatgggagttgggaatgcccgaagccagtgncctaaccgaaaggaagga

SEQ ID NO: 12 - DNA, 1393 bp, synthetic construct
  misc_feature 1..1393: note = Synthetic: FBI00019
  source 1..1393: mol_type = other DNA
tgcagtcgaggggcagcgggnntgaagctntgcttcaatcgccggcgaccggcgcangggtgcgtaacgcgtatgcaacctacccagaacagggggataacactgagaaattggtactaatatcccataacatcataaggggcatcccttttggttgaaaactccggtggttctggatgggcatgcgttgtattagctagttggtgaggtaacggctcaccaaggcaacgatacatagggggactgagaggttaaccccccacattggtactgagacacggaccaaactccnacgggaggcagcagtgaggaatattggtcaatggacgcaagtctgaaccagccatgccgcgtgcaggaagacggctctatgagttgtaaactgcttttgtactagggtaaactcagatacgtgtatctgactgaaagtatagtacgaataaggatcggctaactccgtgccagcagccgcggtaatacggaggattcaagcgttatccggatttattgggtttaaagggtgcgtaggcggtttgataagttagaggtgaaataccggtgcttaacaccggaactgcctctaatactgttgagctagagagtagttgcggtaggcggaatgtatggtgtagcggtgaaatgcttagagatcatacagaacaccgattgcgaaggcagcttaccaaactatatctgacgttgaggcacgaaagcgtggggagcaaacaggattagataccctggtagtccacgcagtaaacgatgataactcgctgtcggcgatacacagtcggtggctaagcgaaagcgataagttatccacctggggagtacgttcgcaagaatgaaactcaaaggaattgacgggggcccgcacaagcggaggaacatgtggtttaattcgatgatacgcgaggaaccttacccgggcttgaaagttagtgacggatctggaaacaggtcttcccttcggggcacgaaactaggtgctgcatggttgtcgtcagctcgtgccgtgaggtgtcgggttaagtcccataacgagcgcaacccctaccgttagttgccatcaggtcaagctgggcactctgacgggactgccggtgtaagccgagaggaaggtggggatgacgtcaaatcagcacggcccttacgtccggggccacacacgtgttacaatggtaggtacagagggcagctacccagcgatgggatgcgaatctcgaaagcctatctcagttcggatcggaggctgaaacccgcctccgtgaagttggattcgctagtaatcgcgcatcagccatggcgcggtgaatacgttcccgggccttgtacacaccgcccgtcaagccatggaagctgggggtgcctgaagttcgtgaccgcaaggagc

SEQ ID NO: 13 - DNA, 1390 bp, synthetic construct
  misc_feature 1..1390: note = Synthetic: FBI00020
  source 1..1390: mol_type = other DNA
gtcgaggggcagcntttnagttntgcnttgcaaactggagatggcgaccggcgcacgggtgagtaacacgtatccaacctgccgataactcggggatagcctttcgaaagaaagattaatacccgatggtataatcagaccgcatggtcttgttattaaagaatttcggttatcgatggggatgcgttccattaggcagttggtgaggtaacggctcaccaaaccttcgatggataggggttctgagaggaaggtcccccacattggaactgagacacggtccaaactcctacgggaggcagcagtgaggaatattggtcaatgggcgcaggcctgaaccagccaagtagcgtgaaggatgactgccctatgggttgtaaacttcttttatatgggaataaagttttccacgtgtggaattttgtatgtaccatatgaataaggatcggctaactccgtgccagcagccgcggtaatacggaggatccgagcgttatccggatttattgggtttaaagggagcgtaggtggacagttaagtcagttgtgaaagtttgcggctcaaccgtaaaattgcagttgatactggctgtcttgagtacagtagaggtgggcggaattcgtggtgtagcggtgaaatgcttagatatcacgaagaactccgattgcgaaggcagctcactggactgcaactgacactgatgctcgaaagtgtgggtatcaaacaggattagataccctggtagtccacacagtaaacgatgaatactcgctgtttgcgatatacagtaagcggccaagcgaaagcattaagtattccacctggggagtacgccggcaacggtgaaactcaaaggaattgacgggggcccgcacaagcggaggaacatgtggtttaattcgatgatacgcgaggaaccttacccgggcttaaattgcatttgaatatattggaaacagtatagtcgtaagacaaatgtgaaggtgctgcatggttgtcgtcagctcgtgccgtgaggtgtcggcttaagtgccataacgagcgcaacccttatctttagttactaacaggtcatgctgaggactctagagagactgccgtcgtaagatgtgaggaaggtggggatgacgtcaaatcagcacggcccttacgtccggggctacacacgtgttacaatggggggtacagaaggcagctacctggtgacaggatgctaatcccaaaagcctctctcagttcggatcgaagtctgcaacccgacttcgtgaagctggattcgctagtaatcgcgcatcagccatggcgcggtgaatacgttcccgggccttgtacacaccgcccgtcaagccatgaaagccgggggtacctgaagtacgtaaccgcaaggag

SEQ ID NO: 14 - DNA, 1395 bp, synthetic construct
  misc_feature 1..1395: note = Synthetic: FBI00021
  source 1..1395: mol_type = other DNA
cagtcgaggggcagcatttntangnttgcgttgcaaacgngaagatggcgacgggcggncgggtgagtaacacgtatccaacctgccgataactccggaatagcctttcgaaagaaagattaataccggatagcatacgaatatcgcatgatatttttattaaagaatttcggttatcgatggggatgcgttccattagtttgttggcggggtaacggcccaccaagactacgatggataggggttctgagaggaaggtccnccacattggaactgagacacggtccaaactcctacgggaggcagcagtgaggaatattggtcaatgggcgagagcctgaaccagccaagtagcgtgaaggatgaaggctctatgggtcgtaaacttcttttatatgggaataaagttttccacgtgtggaattttgtatgtaccatatgaataaggatcggctaactccgtgccagcagccgcggtaatacggaggatccgagcgttatccggatttattgggtttaaagggagcgtaggtggattgttaagtcagttgtgaaagtttgcggctcaaccgtaaaattgcagttgaaactggcagtcttgagtacagtagaggtgggcggaattcgtggtgtagcggtgaaatgcttagatatcacgaagaactccgattgcgaaggcagctcactagactgttactgacactgatgctcgaaagtgtgggtatcaaacaggattagataccctggtagtccacacagtaaacgatgaatactcgctgtttgcgatatacagtaagcggccaagcgaaagcattaagtattccacctggggagtacgccggcaacggtgaaactcaaaggaattgacgggggcccgcacaagcggaggaacatgtggtttaattcgatgatacgcgaggaaccttacccgggcttaaattgcaacagaatatattggaaacagtatagccgtaaggctgttgtgaaggtgctgcatggttgtcgtcagctcgtgccgtgaggtgtcggcttaagtgccataacgagcgcaacccttatctttagttactaacaggttatgctgaggactctagagagactgccgtcgtaagatgtgaggaaggtggggatgacgtcaaatcagcacggcccttacgtccggggctacacacgtgttacaatggggggtacagaaggcggctacctggtgacaggatgctaatcccaaaaacctctctcagttcggatcgaagtctgcaacccgacttcgtgaagctggattcgctagtaatcgcgcatcagccatggcgcggtgaatacgttcccgggccttgtacacaccgcccgtcaagccatgaaagccgggggtacctgaagtacgtaaccgcaagnagc

SEQ ID NO: 15 - DNA, 1378 bp, synthetic construct
  misc_feature 1..1378: note = Synthetic: FBI00022
  source 1..1378: mol_type = other DNA
tgcaagtcgaggggcagcataatggatagcaatatctatggtggcgaccggcgcacgggtgcgtaacgcgtatgcaacctacctttaacagggggataacactgagaaattggtactaataccccataatatcatagaaggcatcttttatggttgaaaattccgatggttagagatgggcatgcgttgtattagctagttggtggggtaacggctcaccaaggcgacgatacatagggggactgagaggttaaccccccacactggtactgagacacggaccagactcctacgggaggcagcagtgaggaatattggtcaatggacgcaagtctgaaccagccatgccgcgtgcaggatgacggctctatgagttgtaaactgcttttgtacgagggtaaacgcagatacgtgtatctgtctgaaagtatcgtacgaataaggatcggctaactccgtgccagcagccgcggtaatacggaggattcaagcgttatccggatttattgggtttaaagggtgcgtaggcggtttgataagttagaggtgaaatttcggggctcaaccctgaacgtgcctctaatactgttgagctagagagtagttgcggtaggcggaatgtatggtgtagcggtgaaatgcttagagatcatacagaacaccgattgcgaaggcagcttaccaaactatatctgacgttgaggcacgaaagcgtggggagcaaacaggattagataccctggtagtccacgcagtaaacgatgataactcgttgtcggcgatacacagtcggtgactaagcgaaagcgataagttatccacctggggagtacgttcgcaagaatgaaactcaaaggaattgacgggggcccgcacaagcggaggaacatgtggtttaattcgatgatacgcgaggaaccttacccgggcttgaaagttagcgacgattcttgaaagaggatttcccttcggggcgcgaaactaggtgctgcatggttgtcgtcagctcgtgccgtgaggtgtcgggttaagtcccataacgagcgcaacccctaccgttagttgccatcaggtgaagctgggcactctggcgggactgccggtgtaagccgagaggaaggtggggatgacgtcaaatcagcacggcccttacgtccggggctacacacgtgttacaatggtaggtacagagggcagctacccagcgatgggatgcgaatctcgaaagcctatctcagttcggattggaggctgaaacccgcctccatgaagttggattcgctagtaatcgcgcatcagccatggcgcggtgaatacgttcccgggccttgtacacaccgcccgtcaagccatgggagccgggggtgccngaagttcg

SEQ ID NO: 16 - DNA, 1393 bp, synthetic construct
  misc_feature 1..1393: note = Synthetic: FBI00025
  source 1..1393: mol_type = other DNA
gcagtcgnncgaagcacttnnctttgattcttcggatgaagaggtttgtgactgagtggcggacgggtgagtaacgcgtgggtaacctgcctcatacagggggataacagttagaaatgactgctaataccgcataagaccacagagccgcatggctcagagggaaaaactccggtggtatgagatggacccgcgtctgattaggtagttggtggggtaacggcctaccaagccaacgatcagtagccgacctgagagggtgaccggccacattgggactgagacacggcccaaactcctacgggaggcagcagtggggaatattgcacaatgggggaaaccctgatgcagcgacgccgcgtgagcgaagaagtatttcggtatgtaaagctctatcagcagggaagaaaatgacggtacctgactaagaagcaccggctaaatacgtgccagcagccgcggtaatacgtatggtgcaagcgttatccggatttactgggtgtaaagggagcgtagacggctgtgtaagtctgaagtgaaagcccggggctcaaccccgggactgctttggaaactatgcagctagagtgtcggagaggtaagtggaattcccagtgtagcggtgaaatgcgtagatattgggaggaacaccagtggcgaaggcggcttactggacgatgactgacgttgaggctcgaaagcgtggggagcaaacaggattagataccctggtagtccacgccgtaaacgatgactactaggtgtcggggagcaaagctcttcggtgccgcagcaaacgcaataagtagtccacctggggagtacgttcgcaagaatgaaactcaaaggaattgacggggacccgcacaagcggtggagcatgtggtttaattcgaagcaacgcgaagaaccttacctgctcttgacatcccggtgaccggcgtgtaatgacgccttttcttcggaacaccggtgacaggtggtgcatggttgtcgtcagctcgtgtcgtgagatgttgggttaagtcccgcaacgagcgcaacccttatcttcagtagccagcaattcggatgggcactctggagagactgccagggataacctggaggaaggtggggatgacgtcaaatcatcatgccccttatgagcagggctacacacgtgctacaatggcgtaaacaaagggaagcgagcctgcgagggtaagcaaatctcaaaaataacgtctcagttcggattgtagtctgcaactcgactacatgaagctggaatcgctagtaatcgcgaatcagcatgtcgcggtgaatacgttcccgggtcttgtacacaccgcccgtcacaccatgggagttggtaacgcccgaagtcagtgacccaaccgtaagganggag

SEQ ID NO: 17 - DNA, 1527 bp, synthetic construct
  misc_feature 1..1527: note = Synthetic: FBI00027
  source 1..1527: mol_type = other DNA
tggagagtttgatcctggctcaggatgaacgctggcggcgtgcttaacacatgcaagtcgagcgaagcrgytargwwgattcttcggatgattytyrrytgactgagcggcggacgggtgagtaacgcgtgggtgacctgccccataccgggggataacagctggaaacggctgctaataccgcataagcgcacagagctgcatggctcrgtgtgaaaaactccggtggtatgggatgggcccgcgtctgattaggcagttggcggggtaacggcccaccaaaccgacgatcagtagccggcctgagagggcgaccggccacattgggactgagacacggcccaaactcctacgggaggcagcagtggggaatattgcacaatgggggaaaccctgatgcagcgacgccgcgtgagcgaagaagtatttcggtatgtaaagctctatcagcagggaagataatgacggtacctgactaagaagccccggctaactacgtgccagcagccgcggtaatacgtagggggcaagcgttatccggatttactgggtgtaaagggagcgtagacggcaaggcaagtctgatgtgaaaacccagggcttaaccctgggactgcattggaaactgtctggctcgagtgccggagaggtaagcggaattcctagtgtagcggtgaaatgcgtagatattaggaagaacaccagtggcgaaggcggcttactggacggtaactgacgttgaggctcgaaagcgtggggagcaaacaggattagataccctggtagtccacgccgtaaacgatgaatgctaggtgttggggagcaaagctcttcggtgccgccgcaaacgcattaagcattccacctggggagtacgttcgcaagaatgaaactcaaaggaattgacggggacccgcacaagcggtggagcatgtggtttaattcgaagcaacgcgaagaaccttaccaggtcttgacatcccgatgaycggtycgtaayggrrccttctcttcggagcattggagacaggtggtgcatggttgtcgtcagctcgtgtcgtgagatgttgggttaagtcccgcaacgagcgcaacccttatcctcagtagccagcargtaawgytgggcactctgtggagactgccagggataacctggaggaaggtggggatgacgtcaaatcatcatgccccttatgatctgggctacacacgtgctacaatggcgtaaacaaagggaggcaaagccgcgaggtggagcaaatcccaaaaataacgtctcagttcggactgcagtctgcaactcgactgcacgaagctggaatcgctagtaatcgcgaatcagaatgtcgcggtgaatacgttcccgggtcttgtacacaccgcccgtcacaccatgggagttggtaacgcccgaagtcagtgacccaaccgtttacggagggagctgccgaaggcgggactgataactggggtgaagtcgtaacaaggtagccgtagcggaaggtgcggytggatcacctcctt

SEQ ID NO: 18 - DNA, 1520 bp, synthetic construct
  misc_feature 1..1520: note = Synthetic: FBI00029
  source 1..1520: mol_type = other DNA
tggagagtttgatcctggctcaggatgaacgctagcgacaggcttaacacatgcaagtcgaggggcagcrsrggtagcrataccsgsyggcgaccggcgcacgggtgagtaacgcgtatgcaacttgcctatcagagggggataacccggcgaaagtcggactaataccgcatgaagcagggrycccgcatgggratatttgctaaagattcatcgctgatagataggcatgcgttccattaggcagttggcggggtaacggcccaccaaaccgacgatggataggggttctgagaggaaggtcccccacattggtactgagacacggaccaaactcctacgggaggcagcagtgaggaatattggtcaatggccgagaggctgaaccagccaagtcgcgtgagggatgaaggttctatggatcgtaaacctcttttataagggaataaagtgcgggacgtgtcccgttttgtatgtaccttatgaataaggatcggctaactccgtgccagcagccgcggtaatacggaggatccgagcgttatccggatttattgggtttaaagggtgcgtaggcggccttttaagtcagcggtgaaagtctgtggctcaaccatagaattgccgttgaaactggggggcttgagtatgtttgaggcaggcggaatgcgtggtgtagcggtgaaatgcatagatatcacgcagaaccccgattgcgaaggcagcctgccaagccatgactgacgctgatgcacgaaagcgtggggatcaaacaggattagataccctggtagtccacgcagtaaacgatgatcactagctgtttgcgatacactgtaagcggcacagcgaaagcgttaagtgatccacctggggagtacgccggcaacggtgaaactcaaaggaattgacgggggcccgcacaagcggaggaacatgtggtttaattcgatgatacgcgaggaaccttacccgggtttgaacgcattcggaccgaggtggaaacaccttttctagcaatagccgtttgcgaggtgctgcatggttgtcgtcagctcgtgccgtgaggtgtcggcttaagtgccataacgagcgcaacccttgccactagttactaacaggtrawgctgaggactctggtgggactgccagcgtaagctgcgaggaaggcggggatgacgtcaaatcagcacggcccttacatccggggcgacacacgtgttacaatggcgtggacaaagggaggccacctggcgacagggagcgaatccccaaaccacgtctcagttcggatcggagtctgcaacccgactccgtgaagctggattcgctagtaatcgcgcatcagccatggcgcggtgaatacgttcccgggccttgtacacaccgcccgtcaagccatgggagccgggggtacctgaagtccgtaaccgcgaggatcggcctagggtaaaactggtgactggggctaagtcgtaacaaggtagccgtaccggaaggtgcggytggatcacctcctt

SEQ ID NO: 19 - DNA, 1376 bp, synthetic construct
  misc_feature 1..1376: note = Synthetic: FBI00030
  source 1..1376: mol_type = other DNA
tgcagtcgancgatgaaccgccctcgggcggacatgaagtggcgaacgggtgagtaacacgtgaccaacctgcccccctctccgggacaaccttgggaaaccgaggctaataccggatactccctcccctnntcctgcaggggtcgggaaagcccaggcggagggggatggggtcgcggcccattaggtagtaggcggggtaacggcccacctagcccgcgatgggtagccgggttgagagaccgaccggccacattgggactgagatacggcccagactcctacgggaggcagcagtggggaattttgcgcaatgggggcaaccctgacgcagcaacgccgcgtgcgggacgacggcctttcgggttgtaaaccgctttcagcagggaagaaattcgacggtacctgcagaagaagctccggctaactacgtgccagcagccgcggtaatacgtagggagcgagcgttatccggattcattgggcgtaaagagcgcgtaggcggcctctcaagcgggatctctaatccgagggctcaacccccggccggatcccgaactgggaggctcgagttcggtagaggcaggcggaattcccggtgtagcggtggaatgcgcagatatcgggaagaacaccgatggcgaaggcagcctgctgggccgcaactgacgctgaggcgcgaaagctaggggagcgaacaggattagataccctggtagtcctagccgtaaacgatggatactaggtgtggggggctccgccctccgtgccgcagccaacgcattaagtatcccgcctggggagtacggccgcaaggctaaaactcaaaggaattgacgggggcccgcacaagcagcggagcatgtggcttaattcgaagcaacgcgaagaaccttaccagggcttgacatggacgtgaagccggggaaacccggtggccgagaggagcgtccgcaggtggtgcatggctgtcgtcagctcgtgtcgtgagatgttgggttaagtcccgcaacgagcgcaacccctgccccatgttgccagcattaggttggggactcatgggggactgccggcgtcaagccggaggaaggtggggacgacgtcaagtcatcatgccctttatgccctgggctgcacacgtgctacaatggccggtacaacgggctgcgagaccgcgaggtcgagcgaatccctcaaagccggccccagttcggatcggaggctgcaacccgcctccgtgaagtcggagttgctagtaatcgcggatcagcatgccgcggtgaatacgttcccgggccttgtacacaccgcccgtcacaccacccgagtcgtctgcacccgaagccgccggccgaacccgcaaggggcggaggcnt

SEQ ID NO: 20 - DNA, 1391 bp, synthetic construct
  misc_feature 1..1391: note = Synthetic: FBI00032
  source 1..1391: mol_type = other DNA
tcgaacgaaacaccttatttgattttcttcggaactgaagatttggtgattgagtggcggacgggtgagtaacgcgtgggtaacctgccctgtacagggggataacagtcagaaatgactgctaataccgcataagaccacagcaccgcatggtgcaggggtaaaaactccggtggtacaggatggacccgcgtctgattagctggttggtgaggtaacggctcaccaaggcgacgatcagtagccggcttgagagagtgaacggccacattgggactgagacacggcccaaactcctacgggaggcagcagtggggaatattgcacaatgggggaaaccctgatgcagcgacgccgcgtgagtgaagaagtatctcggtatgtaaagctctatcagcagggaagaaaatgacggtacctgactaagaagccccggctaactacgtgccagcagccgcggtaatacgtagggggcaagcgttatccggaattactgggtgtaaagggtgcgtaggtggtatggcaagtcagaagtgaaaacccagggcttaactctgggactgcttttgaaactgtcagactggagtgcaggagaggtaagcggaattcctagtgtagcggtgaaatgcgtagatattaggaggaacatcagtggcgaaggcggcttactggactgaaactgacactgaggcacgaaagcgtggggagcaaacaggattagataccctggtagtccacgccgtaaacgatgaatactaggtgtcggggccgtagaggcttcggtgccgcagccaacgcagtaagtattccacctggggagtacgttcgcaagaatgaaactcaaaggaattgacggggacccgcacaagcggtggagcatgtggtttaattcgaagcaacgcgaagaaccttacctggtcttgacatccttctgaccggtccttaaccggacctttccttcgggacaggagtgacaggtggtgcatggttgtcgtcagctcgtgtcgtgagatgttgggttaagtcccgcaacgagcgcaacccctatctttagtagccagcatataaggtgggcactctagagagactgccagggataacctggaggaaggtggggacgacgtcaaatcatcatgccccttatgaccagggctacacacgtgctacaatggcgtaaacagagggaagcagcctcgtgagagtgagcaaatcccaaaaataacgtctcagttcggattgtagtctgcaactcgactacatgaagctggaatcgctagtaatcgcgaatcagaatgtcgcggtgaatacgttcccgggtcttgtacacaccgcccgtcacaccatgggagtcagtaacgcccgaagtcannnacccaaccgtaaggagggag

SEQ ID NO: 21 - DNA, 1526 bp, synthetic construct
  misc_feature 1..1526: note = Synthetic: FBI00033
  source 1..1526: mol_type = other DNA
tggagagtttgatcctggctcaggatgaacgctggcggcgtgcctaacacatgcaagtcgaacgaggyawwtdrrwtgaagttttcggayrgattyywakwtaccgagtggcggacgggtgagtaacgcgtgggtaacctgcctcatacagggggataacggttagaaatgactgctaataccgcataagcgcacagtaccgcatggtacggtgtgaaaaactccggtggtatgagatggacccgcgtctgattagctagttggtgrggtaacggcccaccaaggcgacgatcagtagccgacctgagagggtgaccggccacattgggactgagacacggcccagactcctacgggaggcagcagtgggggatattgcacaatggaggaaactctgatgcagcgacgccgcgtgagtgaagaagtatttcggtatgtaaagctctatcagcagggaagaaaatgacggtacctgactaagaagccccggctaactacgtgccagcagccgcggtaatacgtagggggcaagcgttatccggatttactgggtgtaaagggagcgtagacggcgacgcaagtctgaagtgaaataccygggctcaacctgggaactgctttggaaactgtgttgctagagtgctggagaggtaagcggaattcctagtgtagcggtgaaatgcgtagatattaggaagaacaccagtggcgaaggcggcttactggacagtaactgacgttgaggctcgaaagcgtggggagcaaacaggattagataccctggtagtccacgccgtaaacgatgaatactaggtgttggtgagcaaagctcatcggtgccgccgcaaacgcaataagtattccacctggggagtacgttcgcaagaatgaaactcaaaggaattgacggggacccgcacaagcggtggagcatgtggtttaattcgaagcaacgcgaagaaccttaccaaatcttgacatccctctgaaamryyyttaatcgrgykcctccttcgggacagaggwgacaggtggtgcatggttgtcgtcagctcgtgtcgtgagatgttgggttaagtcccgcaacgagcgcaacccctattgtcagtagccagcaggtmaagctgggcactctgatragactgccagggataacctggaggaaggtggggatgacgtcaaatcatcatgccccttatgatttgggctacacacgtgctacaatggcgtaaacaaagagaagcgagcctgygagggggagcaaatctcaaaaataacgtctcagttcggattgtagtctgcaactcgactacatgaagctggaatcgctagtaatcgcagatcagaatgctgcggtgaatacgttcccgggtcttgtacacaccgcccgtcacaccatgggagtcggaaatgcccgaagccagtgacccaagcgaaagcagggagctgtcgaaggcaggtctgataactggggtgaagtcgtaacaaggtagccgtatcggaaggtgcggytggatcacctcctt

SEQ ID NO: 22 - DNA, 1392 bp, synthetic construct
  misc_feature 1..1392: note = Synthetic: FBI00034
  source 1..1392: mol_type = other DNA
antcgaacgaagcatttacnacagatttcttcggattgaagtnntttatgactgagtggcggacgggtgagtaacgcgtgggtaacctgccttgtactgggggatagcagctggaaacggctggtaataccgcataagcgcacaatgttgcatgacatggtgtgaaaaactccggtggtataagatggacccgcgtctgattagctagttggtgagataacagcccaccaaggcgacgatcagtagccgacctgagagggtgaccggccacattgggactgagacacggcccagactcctacgggaggcagcagtggggaatattgcacaatggaggaaactctgatgcagcgacgccgcgtgagtgaagaagtaattcgttatgtaaagctctatcagcagggaagatagtgacggtacctgactaagaagctccggctaaatacgtgccagcagccgcggtaatacgtatggagcaagcgttatccggatttactgggtgtaaagggagtgtaggtggccatgcaagtcagaagtgaaaatccggggctcaaccccggaactgcttttgaaactgtaaggctggagtgcaggaggggtgagtggaattcctagtgtagcggtgaaatgcgtagatattaggaggaacaccagtggcgaaggcggctcactggactgtaactgacactgaggctcgaaagcgtggggagcaaacaggattagataccctggtagtccacgccgtaaacgatgaatactaggtgtcggggctcataagngcttcggtgccgcagcaaacgcaataagtattccacctggggagtacgttcgcaagaatgaaactcaaaggaattgacggggacccgcacaagcggtggagcatgtggtttaattcgaagcaacgcgaagaaccttaccaagtcttgacatcccactgaccggacagtaatgtgtcctttccttcgggacagtggtgacaggtggtgcatggttgtcgtcagctcgtgtcgtgagatgttgggttaagtcccgcaacgagcgcaacccctatccttagtagccagcagtaagatgggcactctagggagactgccagggataacctggaggaaggtggggatgacgtcaaatcatcatgccccttatgacttgggctacacacgtgctacaatggcgtaaacaaagtgaagcgaagtcgtgaggccaagcaaatcacaaaaataacgtctcagttcggattgtagtctgcaactcgactacatgaagctggaatcgctagtaatcgcagatcagaatgctgcggtgaatacgttcccgggtcttgtacacaccgcccgtcacaccatgggagtcgaaaatgcccgaagtcggtgacctaacgtaagaaggagc

SEQ ID NO: 23 - DNA, 1382 bp, synthetic construct
  misc_feature 1..1382: note = Synthetic: FBI00038
  source 1..1382: mol_type = other DNA
tgcagtcgacgagagaagattggtgcttgcactgatccgatcgagtggcggacgggtgagtaacgcgtgggtaacctgcctcatacaggggaataacagttggaaacggctgctaaaaccgcataagcgcacggaatcgcatgatacagtgtgaaaaactccggtggtatgagatggacccgcgtctgattagctagttggtgaggtaacggcccaccaaggcaacgatcagtagccggcctgagagggtgaacggccacattgggactgagacacggcccaaactcctacgggaggcagcagtggggaatattgcacaatgggggaaaccctgatgcagcgacgccgcgtgagtgatgaagtatttcggtatgtaaagctctatcagcagggaagataatgacggtacctgactaagaagcaccggctaaatacgtgccagcagccgcggtaatacgtatggtgcaagcgttatccggatttactgggtgtaaagggtgcgtaggtggtgagacaagtctgaagtgaaaatccggggcttaaccccggaactgctttggaaactgcctgactagagtacaggagaggtaagtggaattcctagtgtagcggtgaaatgcgtagatattaggaggaacaccagtggcgaaggcgacttactggactgctactgacactgaggcacgaaagcgtggggagcaaacaggattagataccctggtagtccacgccgtaaacgatgaatactaggtgtcggggcccaaagggcttcggtgccgcagccaacgcaataagtattccacctggggagtacgttcgcaagaatgaaactcaaaggaattgacggggacccgcacaagcggtggagcatgtggtttaattcgaagcaacgcgaagaaccttaccaagtcttgacatcctgctgaccgttccttaaccggaattttccttcgggacagcagagacaggtggtgcatggttgtcgtcagctcgtgtcgtgagatgttgggttaagtcccgcaacgagcgcaacccctatttccagtagccagcagtcagatgggcactctggagagactgccggggataacccggaggaaggtggggatgacgtcaaatcatcatgccccttatgatttgggctacacacgtgctacaatggcagttacaaagagaagcgaaactgtgaagtggagcaaacctcaaaaaggctgtctcagttcggattgtagtctgcaactcgacnacatgaagctggaatcgctagtaatcgcagatcagaatgctgcggtgaatacgttcccgggtcttgtacacaccgcccgtcacaccatgggagtcggaaacgcccgaagtcagtgacccaaccgcaaggagggagc

SEQ ID NO: 24 - DNA, 1400 bp, synthetic construct
  misc_feature 1..1400: note = Synthetic: FBI00040
  source 1..1400: mol_type = other DNA
gtcgaacgtgaaagtccttcgggacnagtaaagtggcgcacgggtgagtaacgcgtggataatctacccttaagatggggataacggctggaaacggtcgctaataccgaatacgctcccgattttatcattggggggaaagatggcctctgcttgcaagctatcgcttaaggatgagtccgcgtcccattagctagttggcggggtaacggcccaccaaggcaacgatgggtagccggtctgagaggatgaccggccacactggaactggaacacggtccaaactcctacgggaggcagcagtggggaatattgcgcaatgggcgaaagcctgacgcagcgacgccgcgtgagggatgaaggttctcggatcgtaaacctctgtcaggggggaaaaaaccccctcgtgtgaataatgcgagggcttgacggtacccccaaaggaagcaccggctaactccgtgccagcagccgcggtaatacggagggtgcaagcgttaatcggaatcactgggcgtaaagcgcacgtaggcggcttggtaagtcaggggtgaaatcccacagcccaactgtggaactgcctttgatactgccaggcttgagtaccggagagggtggcggaattccaggtgtaggagtgaaatccgtagatatctggaggaacaccggtggcgaaggcggccacctggacggtaactgacgctgaggtgcgaaagcgtgggtagcaaacaggattagataccctggtagtccacgctgtaaacgatgggtgctgggtgctgggatgtatgtctcggtgccgtagctaacgcgataagcaccccgcctggggagtacggtcgcaaggctgaaactcaaagaaattgacgggggcccgcacaagcggtggagtatgtggtttaattcgatgcaacgcgaagaaccttacccaggcttgacatctagggaacccttcggaaatgaaggggtgcccttcggggagccctaagacaggtgctgcatggctgtcgtcagctcgtgccgtgaggtgttgggttaagtcccgcaacgagcgcaacccctatcttcagttgccagcaggtaaggctgggcactctggagagaccgccccggtcaacggggaggaaggtggggacgacgtcaagtcatcatggcccttacgcctggggctacacacgtactacaatggcgcgcacaaagggtagcgagaccgcgaggtggagccaatcccaaaaaacgcgtcccagtccggattggagtctgcaactcgactccatgaagtcggaatcgctagtaattcgagatcagcatgctcgggtgaatgcgttcccgggccttgtacacaccgcccgtcacaccacgaaagtcggttttacccgaagccggtgagctaactntgc

SEQ ID NO: 25 - DNA, 1525 bp, synthetic construct
  misc_feature 1..1525: note = Synthetic: FBI00043
  source 1..1525: mol_type = other DNA
tggagagtttgatcctggctcaggatgaacgctggcggcgtgcttaacacatgcaagtcgaacgggatcccgggggttcgctcccgggtgagagtggcgaacgggtgagtaatgcgtgaccgacctgccccatacaccggaatagctcctggaaacgggtggtaatgccggatgctccggttggatgcatgtccttccgggaaagattccatcggtatgggatggggtcgcgtcctatcagcttgatggcggggtaacggcccaccatggcctcgacgggtagccggcctgagagggcgaccggccacattgggactgagatacggcccagactcctacgggaggcagcagtggggaatattgcacaatgggcgcaagcctgatgcagcgacgccgcgtgcgggatggaggccttcgggttgtaaaccgcttttgatcgggagcaagcccttcggggtgagtgtacccttcgaataagcaccggctaactacgtgccagcagccgcggtaatacgtagggtgcaagcgttatccggaattattgggcgtaaagggctcgtaggcggttcgtcgcgtccggtgtgaaagcccatcgcttaacggtgggtctgcgccgggtacgggcgggctggagtgcggtaggggagactggaattcccggtgtaacggtggaatgtgtagatatcgggaagaacaccaatggcgaaggcaggtctctgggccgtcactgacgctgaggagcgaaagcgtggggagcgaacaggattagataccctggtagtccacgccgtaaacggtggatgctggatgtggggcccgttccacgggttccgtgtcggagctaacgcgttaagcatcccgcctggggagtacggccgcaaggctaaaactcaaagaaattgacgggggcccgcacaagcggcggagcatgcggattaattcgatgcaacgcgaagaaccttacctgggcttgacatgttcccgacggtcgtagagatatggcctcccttcggggcgggttcacaggtggtgcatggtcgtcgtcagctcgtgtcgtgagatgttgggttaagtcccgcaacgagcgcaaccctcgccctgtgttgccagcacgtcgtggtgggaactcacgggggaccgccggggtcaactcggaggaaggtggggatgacgtcagatcatcatgccccttacgtccagggcttcacgcatgctacaatggccggtacagcgggatgcgacatggcgacatggagcggatccctgaaaaccggtctcagttcggattggagtctgcaacccgactccatgaaggcggagtcgctagtaatcgcggatcagcaacgccgcggtgaatgcgttcccgggccttgtacacaccgcccgtcaagtcatgaaagtgggcagcacccgaagccggtggcctaaccccttgtgggatggagccgtctaaggtgaggctcgtgattgggactaagtcgtaacaaggtagccgtaccggaaggtgcggytggatcacctcctt

SEQ ID NO: 26 - DNA, 1525 bp, synthetic construct
  misc_feature 1..1525: note = Synthetic: FBI00044
  source 1..1525: mol_type = other DNA
tggagagtttgatcctggctcaggatgaacgctggcggcgtgcttaacacatgcaagtcgaacgggaawtayttyattgaracttcggtsgatttratytawttctagtggcggacgggtgagtaacgcgtgggtaacctgccttatacagggggataacagtcagaaatggctgctaataccgcataagcgcacagagctgcatggctcagtgtgaaaaactccggtggtataagatggacccgcgttggattagcttgttggtggggtaacggccyaccaaggcgacgatccatagccggcctgagagggtgaacggccacattgggactgagacacggcccagactcctacgggaggcagcagtggggaatattgcacaatgggggaaaccctgatgcagcgacgccgcgtgaaggaagaagtatctcggtatgtaaacttctatcagcagggaagatagtgacggtacctgactaagaagccccggctaactacgtgccagcagccgcggtaatacgtagggggcaagcgttatccggatttactgggtgtaaagggagcgtagacggtgtggcaagtctgatgtgaaaggcatgggctcaacctgtggactgcattggaaactgtcatacttgagtgccggaggggtaagcggaattcctagtgtagcggtgaaatgcgtagatattaggaggaacaccagtggcgaaggcggcttactggacggtaactgacgttgaggctcgaaagcgtggggagcaaacaggattagataccctggtagtccacgccgtaaacgatgaatactaggtgtcgggkrgcakrgcymttcggtgccgtcgcaaacgcagtaagtattccacctggggagtacgttcgcaagaatgaaactcaaaggaattgacggggacccgcacaagcggtggagcatgtggtttaattcgaagcaacgcgaagaaccttaccaartcttgacatccbyctgaccgrwmyttaaycgkwycttyycttcggrrcagrvgwgacaggtggtgcatggttgtcgtcagctcgtgtcgtgagatgttgggttaagtcccgcaacgagcgcaacccctatcctcagtagccagcatttaaggtgggcactctggggagactgccagggataacctggaggaaggcggggatgacgtcaaatcatcatgccccttatgatttgggctacacacgtgctacaatggcgtaaacaaagggaagcgagatcgtgagatggagcaaatcccaaaaataacgtcccagttcggactgtagtctgcaacccgactacacgaagctggaatcgctagtaatcgcggatcagaatgccgcggtgaatacgttcccgggtcttgtacacaccgcccgtcacaccatgggagtcagtaacgcccgaagtcagtgacctaactgcaaagaaggagctgccgaaggcgggaccgatgactggggtgaagtcgtaacaaggtagccgtatcggaaggtgcggytggatcacctcctt

SEQ ID NO: 27 - DNA, 1393 bp, synthetic construct
  misc_feature 1..1393: note = Synthetic: FBI00046
  source 1..1393: mol_type = other DNA
tgcagtcgaggggcatcagtttggtttgcttgcaaaccaaagctggcgaccggcgcacgggtgagtaacacgtatccaacctgcctcatactcggggatagcctttcgaaagaaagattaatatccgatagcatatatttcccgcatgggttttatattaaagaaattcggtatgagatggggatgcgttccattagtttgttggcggggtaacggcccaccaagactacgatggataggggttctgagaggaaggtcccccacattggaactgagacacggtccaaactcctacgggaggcagcagtgaggaatattggtcaatggacgcgagtctgaaccagccaagtagcgtgaaggatgactgccctatgggttgtaaacttcttttatatgggaataaagtggtccacgtgtggatttttgtatgtaccatatgaataaggatcggctaactccgtgccagcagccgcggtaatacggaggatccgagcgttatccggatttattgggtttaaagggagcgtaggcggattgttaagtcagttgtgaaagtttgcggctcaaccgtaaaattgcagttgatactggcagtcttgagtgcagtagaggtgggcggaattcgtggtgtagcggtgaaatgcttagatatcacgaagaactccgattgcgaaggcagctcactggagtgtaactgacgctgatgctcgaaagtgtgggtatcaaacaggattagataccctggtagtccacacagtaaacgatgaatactcgctgtttgcgatatacagtaagcggccaagcgaaagcattaagtattccacctggggagtacgccggcaacggtgaaactcaaaggaattgacgggggcccgcacaagcggaggaacatgtggtttaattcgatgatacgcgaggaaccttacccgggcttaaattgcaaatgaattatggggaaacccatacgccgcaaggcatttgtgaaggtgctgcatggttgtcgtcagctcgtgccgtgaggtgtcggcttaagtgccataacgagcgcaacccttatcttcagttactaacaggtcatgctgaggactctggagagactgccgtcgtaagatgtgaggaaggtggggatgacgtcaaatcagcacggcccttacgtccggggctacacacgtgttacaatggggggtacagaaggccgctacctggtgacaggatgccaatcccaaaaacctctctcagttcggatcgaagtctgcaacccgacttcgtgaagctggattcgctagtaatcgcgcatcagccatggcgcggtgaatacgttcccgggccttgtacacaccgcccgtcaagccatgaaagccgggggtacctgaagtacgtaaccgcaaggagc

SEQ ID NO: 28 - DNA, 1410 bp, synthetic construct
  misc_feature 1..1410: note = Synthetic: FBI00047
  source 1..1410: mol_type = other DNA
anncatgcagtcgaacgaagcatttangaacangattacttcggtgtntgaagttnnttatgactgagtggcggacgggtgagtaacgcgtgggtaacctgccttgtactgggggatagcagctggaaacggctggtaataccgcataagcgcacaatgttgcatgacatggtgtgaaaaactccggtggtataagatggacccgcgtctgattagctagttggtgagataacagcccaccaaggcgacgatcagtagccgacctgagagggtgaccggccacattgggactgagacacggcccagactcctacgggaggcagcagtggggaatattgcacaatggaggaaactctgatgcagcgacgccgcgtgagtgaagaagtaattcgttatgtaaagctctatcagcagggaagatagtgacggtacctgactaagaagctccggctaaatacgtgccagcagccgcggtaatacgtatggagcaagcgttatccggatttactgggtgtaaagggagtgtaggtggccatgcaagtcagaagtgaaaatccggggctcaaccccggaactgcttttgaaactgtaaggctggagtgcaggaggggtgagtggaattcctagtgtagcggtgaaatgcgtagatattaggaggaacaccagtggcgaaggcggctcactggactgtaactgacactgaggctcgaaagcgtggggagcaaacaggattagataccctggtagtccacgccgtaaacgatgaatactaggtgtcggggcccataagggcttcggtgccgcagcaaacgcaataagtattccacctggggagtacgttcgcaagaatgaaactcaaaggaattgacggggacccgcacaagcggtggagcatgtggtttaattcgaagcaacgcgaagaaccttaccaagtcttgacatcccactgaccggacagtaatgtgtcctttccttcgggacagtggtgacaggtggtgcatggttgtcgtcagctcgtgtcgtgagatgttgggttaagtcccgcaacgagcgcaacccctatccttagtagccagcagtaagatgggcactctagggagactgccagggataacctggaggaaggtggggatgacgtcaaatcatcatgccccttatgacttgggctacacacgtgctacaatggcgtaaacaaagtgaagcgaagtcgtgaggccaagcaaatcacaaaaataacgtctcagttcggattgtagtctgcaactcgactacatgaagctggaatcgctagtaatcgcagatcagaatgctgcggtgaatacgttcccgggtcttgtacacaccgcccgtcacaccatgggagtcgaaaatgcccgaagtcggtgacctaacgaaagaaggagccgccga

SEQ ID NO: 29 - DNA, 1528 bp, synthetic construct
  misc_feature 1..1528: note = Synthetic: FBI00048
  source 1..1528: mol_type = other DNA
tggagagtttgatcctggctcaggatgaacgctggcggcgtgcttaacacatgcaagtcgagcgaagcrgytargwtgatttcttcggattgattcttractgactgagcggcggacgggtgagtaacgcgtgggtgacctgccccataccgggggataacagctggaaacggctgctaataccgcataagcgcacagagctgcatggctcrgtgtgaaaaactccggtggtatgggatgggcccgcgtctgattaggcagttggcggggtaacggcccaccaaaccgacgatcagtagccggcctgagagggcgaccggccacattgggactgagacacggcccaaactcctacgggaggcagcagtggggaatattgcacaatgggggaaaccctgatgcagcgacgccgcgtgagcgaagaagtatttcggtatgtaaagctctatcagcagggaagataatgacggtacctgactaagaagccccggctaactacgtgccagcagccgcggtaatacgtagggggcaagcgttatccggatttaytgggygtaaagggmkcgtagrcggcaaggcaagtctgatgtgaaaacccagggcttaaccctgggactgcattggaaactgtctggctygagtgccggagaggtaagcggaattcctagtgtagcggtgaaatgcgtagatattaggaagaacaccagtggcgaaggcggcttactggacggtaactgacgttgaggctcgaaagcgtggggagcaaacaggattagataccctggtagtccacgccgtaaacgatgaatgctaggtgttggggagcaaagctcttcggtgccgccgcaaacgcattaagcattccacctggggagtacgttcgcaagaatgaaactcaaaggaattgacggggacccgcacaagcggtggagcatgtggtttaattcgaagcaacgcgaagaaccttaccaggtcttgacatcccgatgaccggyycgtaayggrrccttctcttcggagcattggagacaggtggtgcatggttgtcgtcagctcgtgtcgtgagatgttgggttaagtcccgcaacgagcgcaacccttatcctcagtagccagcaggtmaagctgggcactctgtggagactgccagggataacctggaggaaggtggggatgacgtcaaatcatcatgccccttatgatctgggctacacacgtgctacaatggcgtaaacaaagggaggcaaagccgcgaggtggagcaaatcccaaaaataacgtctcagttcggactgcagtctgcaactcgactgcacgaagctggaatcgctagtaatcgcgaatcagaatgtcgcggtgaatacgttcccgggtcttgtacacaccgcccgtcacaccatgggagttggtaacgcccgaagtcagtgacccaacctttyaggagggagctgccgaaggcgggactgataactggggtgaagtcgtaacaaggtagccgtatcggaaggtgcggytggatcacctcctt

SEQ ID NO: 30 - DNA, 1406 bp, synthetic construct
  misc_feature 1..1406: note = Synthetic: FBI00049
  source 1..1406: mol_type = other DNA
cgggaggaaaagagaagcttgcttctttttgaatctagtggcaaacgggtgagtaacacgtaaacaacctgccttcaggatggggacaacagacggaaacgactgctaataccgaatgtgttccggagaccgcatgatttccggaagaaaggatggcctctatttataagctatcgcctgaagaggggtttgcgtctgattaggcagttggtgaggtaacggcccaccaaacctacgatcagtagccggtctgagaggatgaacggccacactggaactgagacacggtccagactcctacgggaggcagcagtggggaatcttccgcaatggacgaaagtctgacggagcaacgccgcgtgagtgatgacggccttcgggttgtaaagctctgtgatcggggacgaatggctggtatgctaataccatatcagagtgacggtacccgaatagcaagccacggctaactacgtgccagcagccgcggtaatacgtaggtggcaagcgttgtccggaattattgggcgtaaagcgcgcgcaggcggcttcttaagtcccatcttaaaagtgcggggcttaaccccgtgatgggatggaaactgggaggctggagtatcggagaggaaagtggaattcctagtgtagcggtgaaatgcgtagagattaggaagaacaccggtggcgaaggcgactttctggacgacaactgacgctgaggcgcgaaagcgtggggagcaaacaggattagataccctggtagtccacgccgtaaacgatgaatactaggtgtaggaggtatcgaccccttctgtgccggagctaacacaataagtattccgcctgggaagtacgatcgcaagattaaaactcaaaggaattgacgggggcccgcacaagcggtggagtatgtggtttaattcgacgcaacgcgaagaaccttaccaggtcttgacattgactgcaatcccgagaaatcgggagttcccttcggggacaggaaaacaggtggtgcacggctgtcgtcagctcgtgtcgtgagatgttgggttaagtcccgcaacgagcgcaacccctatcttatgttgccagcacgtaatggtgggaactcatgagagaccgccgcggacaacgcggaggaaggcggggatgacgtcaagtcatcatgccccttatgacctgggctacacacgtactacaatgggtgtcaacaaagagaagcaatggggcgacccggagcaaacctcaaaaacacacccccagttcagattgcaggctgcaacccgcctgcatgaagcaggaatcgctagtaatcgcgggtcagcataccgcggtgaatacgttcccgggccttgtacacaccgcccgtcacactatgagagtcagaaacacccgaagccggtgaggta

SEQ ID NO: 31 - DNA, 1519 bp, synthetic construct
  misc_feature 1..1519: note = Synthetic: FBI00050
  source 1..1519: mol_type = other DNA
tggagagtttgatcctggctcaggatgaacgctagctacaggcttaacacatgcaagtcgaggggcatcaggatgtagcaatacattgctggcgaccggcgcacgggtgagtaacacgtatccaacctgccttttactcggggataggctttcgaaagaaagattaatacccgatggcataatattyccgcatgggrttattattaaagaatttcggtaaaagatggggatgcgttccattaggcagttggcggggtaacggcccaccaaaccttcgatggataggggttctgagaggaaggtcccccacattggaactgagacacggtccaaactcctacgggaggcagcagtgaggaatattggtcaatgggcgagagcctgaaccagccaagtagcgtgaaggatgactgccctatgggttgtaaacttcttttatatgggaataaagtgagccacgtgtggctttttgtatgtaccttatgaataaggatcggctaactccgtgccagcagccgcggtaatacggaggatccgagcgttatccggatttattgggtttaaagggagcgtaggtggacatgtaagtcagttgtgaaagtttgcggctcaaccgtaaaattgcagttgatactgtgtgtcttgagtacagtagaggtgggcggaattcgtggtgtagcggtgaaatgcttagatatcacgaagaactccgattgcgaaggcagctcactggactggtaactgacactgaggctcgaaagtgtgggtatcaaacaggattagataccctggtagtccacacagtaaacgatgaatactcgctgtttgcgatatacagcaagcggccaagcgaaagcattaagtattccacctggggagtacgccggcaacggtgaaactcaaaggaattgacgggggcccgcacaagcggaggaacatgtggtttaattcgatgatacgcgaggaaccttacccgggcttaaattgcatttgaataatttggaaacagattagccgcaaggcaaatgtgaaggtgctgcatggttgtcgtcagctcgtgccgtgaggtgtcggcttaagtgccataacgagcgcaacccttatctttagttactaacaggtcatgctgaggactctagagagactgccgtcgtaagatgtgaggaaggtggggatgacgtcaaatcagcacggcccttacgtccggggctacacacgtgttacaatggggggtacagaaggcagctacacggcgacgtgatgctaatcccaaaagcctctctcagttcggatcgaagtctgcaacccgacttcgtgaagctggattcgctagtaatcgcgcatcagccacggcgcggtgaatacgttcccgggccttgtacacaccgcccgtcaagccatgggagccgggggtacctgaagtacgtaaccgcaaggagcgtcctagggtaaaactggtgactggggctaagtcgtaacaaggtagccgtaccggaaggtgcggytggatcacctcctt

SEQ ID NO: 32 - DNA, 1526 bp, synthetic construct
  misc_feature 1..1526: note = Synthetic: FBI00051
  source 1..1526: mol_type = other DNA
tggagagtttgatcctggctcaggatgaacgctggcggcgtgcttaacacatgcaagtcgagcgaagcacwtaagtttgattcttcggatgaagacttttgtgactgagcggcggacgggtgagtaacgcgtgggtaacctgcctcatacagggggataacagttagaaatggctgctaataccgcataagaccacrgtactgcatggtacagtggtaaaaactccggtggtatgagatggacccgcgtctgattaggtagttggtgaggtaacggcycaccaagccgacgatcagtagccgacctgagagggtgaccggccacattgggactgagacacggcccagactcctacgggaggcagcagtggggaatattgcacaatgggcgaaagcctgatgcagcgacgccgcgtgaaggatgaagtatttcggtatgtaaacttctatcagcagggaagaaaatgacggtacctgactaagaagccccggctaactacgtgccagcagccgcggtaatacgtagggggcaagcgttatccggatttactgggtgtaaagggagcgtagacggctgtgcaagtctgaagtgaaaggcatgggctcaacctgtggactgctttggaaactgtgcagctagagtgtcggagaggtaagtggaattcctagtgtagcggtgaaatgcgtagatattaggaggaacaccagtggcgaaggcggcttactggacgatgactgacgttgaggctcgaaagcgtggggagcaaacaggattagataccctggtagtccacgccgtaaacgatgactgctaggtgtcgggtagcaaagctattcggtgccgcagctaaygcaataagcagtccacctggggagtacgttcgcaagaatgaaactcaaaggaattgacggggacccgcacaagcggtggagcatgtggtttaattcgaagcaacgcgaagaaccttacctgatcttgacatcccgatgaccgcttcgtaatggaagyttttcttcggaacatcggtgacaggtggtgcatggttgtcgtcagctcgtgtcgtgagatgttgggttaagtcccgcaacgagcgcaacccttatcttcagtagccagcattwaggatgggcactctggagagactgccagggataacctggaggaaggtggggatgacgtcaaatcatcatgccccttatgaccagggctacacacgtgctacaatggcgtaaacaaagggaagcagagccgcgaggccgagcaaatctcaaaaataacgtctcagttcggattgtagtctgcaactcgactacatgaagctggaatcgctagtaatcgcagatcagaatgctgcggtgaatacgttcccgggtcttgtacacaccgcccgtcacaccatgggagtcagtaacgcccgaagtcagtgacccaaccgaaaggagggagctgccgaaggtgggaccgataactggggtgaagtcgtaacaaggtagccgtatcggaaggtgcggytggatcacctcctt

SEQ ID NO: 33 - DNA, 1401 bp, synthetic construct
  misc_feature 1..1401: note = Synthetic: FBI00052
  source 1..1401: mol_type = other DNA
anncatgcagtcgaggggcagcattttagtttgcttgcaaactaaagatggcgaccggcgcacgggtgagtaacacgtatccaacctgccgataactcggggatagcctttcgaaagaaagattaatatccgatagtatattaaaaccgcatggttttactattaaagaatttcggttatcgatggggatgcgttccattagtttgttggcggggtaacggcccaccaagactacgatggataggggttctgagaggaaggtcccccacattggaactgagacacggtccaaactcctacgggaggcagcagtgaggaatattggtcaatggacgagagtctgaaccagccaagtagcgtgaaggatgactgccctatgggttgtaaacttcttttatatgggaataaagtattccacgtgtggaattttgtatgtaccatatgaataaggatcggctaactccgtgccagcagccgcggtaatacggaggatccgagcgttatccggatttattgggtttaaagggagcgtaggtggattgttaagtcagttgtgaaagtttgcggctcaaccgtaaaattgcagttgaaactggcagtcttgagtacagtagaggtgggcggaattcgtggtgtagcggtgaaatgcttagatatcacgaagaactccgattgcgaaggcagctcactagactgcaactgacactgatgctcgaaagtgtgggtatcaaacaggattagataccctggtagtccacacagtaaacgatgaatactcgctgtttgcgatatacagtaagcggccaagcgaaagcattaagtattccacctggggagtacgccggcaacggtgaaactcaaaggaattgacgggggcccgcacaagcggaggaacatgtggtttaattcgatgatacgcgaggaaccttacccgggcttaaattgcatttgaatatagtggaaacattatagccgcaaggcaaatgtgaaggtgctgcatggttgtcgtcagctcgtgccgtgaggtgtcggcttaagtgccataacgagcgcaacccttatctttagttactaacaggttatgctgaggactctagagagactgccgtcgtaagatgtgaggaaggtggggatgacgtcaaatcagcacggcccttacgtccggggctacacacgtgttacaatggggggtacagaaggcagctacctggcgacaggatgctaatcccaaaaacctctctcagttcggatcgaagtctgcaacccgacttcgtgaagctggattcgctagtaatcgcgcatcagccatggcgcggtgaatacgttcccgggccttgtacacaccgcccgtcaagccatgaaagccgggggtacctgaagtacgtaaccgcaaggagcnnc

SEQ ID NO: 34 - DNA, 1397 bp, synthetic construct
  misc_feature 1..1397: note = Synthetic: FBI00053
  source 1..1397: mol_type = other DNA
tgcagtcgancgaagcatttaagacggattctttcgggatgaagacttttatgactgagtggcggacgggtgagtaacgcgtgggtaacctgcctcacacagggggatagcagttggaaacggctgataataccgcataagcgcacagtaccgcatggtacagtgtgaaaaactccggtggtgtgagatggacccgcgtctgattagcttgttggcagggtaacggcctaccaaggcaacgatcagtagccgacctgagagggtgaccggccacattgggactgagacacggcccagactcctacgggaggcagcagtggggaatattgcacaatggaggaaactctgatgcagcgacgccgcgtgagtgaagaagtaattcgttatgtaaagctctatcagcagggaagatagtgacggtacctgactaagaagctccggctaaatacgtgccagcagccgcggtaatacgtatggagcaagcgttatccggatttactgggtgtaaagggagtgtaggtggcatcacaagtcagaagtgaaagcccggggctcaaccccgggactgcttttgaaactgtggagctggagtgcaggagaggcaagtggaattcctagtgtagcggtgaaatgcgtagatattaggaggaacaccagtggcgaaaggcggcttgctggactgtaactgacactgaggctcgaaagcgtggggagcaaacaggattagataccctggtagtccacgccgtaaacgatgaatactaggtgtcggggctcataagagcttcggtgccgcagcaaacgcaataagtattccacctggggagtacgttcgcaagaatgaaactcaaaggaattgacggggacccgcacaagcggtggagcatgtggtttaattcgaagcaacgcgaagaaccttaccaagtcttgacatcctcttgcccggtcagtaatgtgaccttttcttcggaacaagagtgacaggtggtgcatggttgtcgtcagctcgtgtcgtgagatgttgggttaagtcccgcaacgagcgcaacccctattcttagtagccagcatttaaggtgggcactctaggaagactgccagggataacctggaggaaggtggggatgacgtcaaatcatcatgccccttatgacttgggctacacacgtgctacaatggcgtaaacaaagtgaagcgagagtgtgagcttaagcaaatcacaaaaataacgtctcagttcggattgtagtctgcaactcgactacatgaagctggaatcgctagtaatcgcgaatcagaatgtcgcggtgaatacgttcccgggtcttgtacacaccgcccgtcacaccatgggagtcggaaatgcccgaagtcggtgacctaacgaaagaaggagc

SEQ ID NO: 35 - DNA, 1387 bp, synthetic construct
  misc_feature 1..1387: note = Synthetic: FBI00056
  source 1..1387: mol_type = other DNA
cntgcagtcgnncgaagcaatcnnaatgaagttttcggatggatttctgattgactgagtggcggacgggtgagtaacgcgtggataacctgcctcacactgggggataacagttagaaatggctgctaataccgcataagcgcacagtaccgcatggtacggtgtgaaaaactccggtggtgtgagatggatccgcgtctgattagttagttggcggggtaacggcccaccaagacagcgatcagtagccgacctgagagggtgaccggccacattgggactgagacacggcccaaactcctacgggaggcagcagtggggaatattgcacaatgggcgaaagcctgatgcagcgacgccgcgtgagtgaagaagtatttcggtatgtaaagctctatcagcagggaagaaactgacggtacctgactaagaagccccggctaactacgtgccagcagccgcggtaatacgtagggggcaagcgttatccggatttactgggtgtaaagggagcgtagacgcgaagcaagtctggagtgaaaacccagggctcaaccctgggactgctttggaaactgttttgctagagtgtcggagaggtaagtggaattcctagtgtagcggtgaaatgcgtagatattaggaggaacaccagtggcgaaggcggcttactggacgataactgacgttgaggctcgaaagcgtggggagcaaacaggattagataccctggtagtccacgccgtaaacgatgaatgctaggtgttggggggcaaagcccttcggtgccgtcgcaaacgcaataagcattccacctgggggagtacgttcgcaagaatgaaactcaaaggaattgacggggacccgcacaagcggtggagcatgtggtttaattcgaagcaacgcgaagaaccttaccaagtcttgacatccccctgaccggtcagtaaagtgacctttccttcgggacaggggagacaggtggtgcatggttgtcgtcagctcgtgtcgtgagatgttgggttaagtcccgcaacgagcgcaacccttatccttagtagccagcaggtaaagctgggcactctagggagactgccagggataacctggaggaaggtggggatgacgtcaaatcatcatgccccttatgatttgggctacacacgtgctacaatggcgtaaacaaagggaagcgaccctgcgaaggcaagcaaatcccaaaaataacgtcccagttcggactgtagtctgcaacccgactacacgaagctggaatcgctagtaatcgcgaatcagaatgtcgcggtgaatacgttcccgggtcttgtacacaccgcccgtcacaccatgggagtcagcaacgcccgaagtcagtgncccaantcg

SEQ ID NO: 36 - DNA, 1399 bp, synthetic construct
  misc_feature 1..1399: note = Synthetic: FBI00057
  source 1..1399: mol_type = other DNA
tgcagtcgagcgaagcacttnngtttgattcttcggatgaagacttttgtgactgagcggcggacgggtgagtaacgcgtgggtaacctgcctcatacagggggataacagttagaaatgactgctaataccgcataagaccacggtaccgcatggtacagtggtaaaaactccggtggtatgagatggacccgcgtctgattaggtagttggtggggtaacggcctaccaagccgacgatcagtagccgacctgagagggtgaccggccacattgggactgagacacggcccagactcctacgggaggcagcagtggggaatattgcacaatggaggaaactctgatgcagcgacgccgcgtgaaggatgaagtatttcggtatgtaaacttctatcagcagggaagaaaatgacggtacctgactaagaagccccggctaactacgtgccagcagccgcggtaatacgtagggggcaagcgttatccggatttactgggtgtaaagggagcgtagacggcacggcaagccagatgtgaaagcccggggctcaaccccgggactgcatttggaactgctgagctagagtgtcggagaggcaagtggaattcctagtgtagcggtgaaatgcgtagatattaggaggaacaccagtggcgaaggcggcttgctggacgatgactgacgttgaggctcgaaagcgtggggagcaaacaggattagataccctggtagtccacgccgtaaacgatgactgctaggtgtcgggtggcaaagccattcggtgccgcagctaacgcaataagcagtccacctggggagtacgttcgcaagaatgaaactcaaaggaattgacggggacccgcacaagcggtggagcatgtggtttaattcgaagcaacgcgaagaaccttacctgatcttgacatcccgatgaccgcttcgtaatggaagctttttcttcggaacatcggtgacaggtggtgcatggttgtcgtcagctcgtgtcgtgagatgttgggttaagtcccgcaacgagcgcaacccctatcttcagtagccagcaggttaagctgggcactctggagagactgccagggataacctggaggaaggtggggatgacgtcaaatcatcatgccccttatgaccagggctacacacgtgctacaatggcgtaaacaaagagaagcgaactcgcgagggtaagcaaatctcaaaaataacgtctcagttcggattgtagtctgcaactcgactacatgaagctggaatcgctagtaatcgcagatcagaatgctgcggtgaatacgttcccgggtcttgtacacaccgcccgtcacaccatgggagtcagtaacgcccgaagtcagtgacccaaccgtaaggnnnagctgc

SEQ ID NO: 37 - DNA, 1350 bp, synthetic construct
  misc_feature 1..1350: note = Synthetic: FBI00059
  source 1..1350: mol_type = other DNA
gntggcgaccggcgcacgggtgagtaacacgtatccaacctgccgattattccgggatagcctttctaaagaaagattaatactggatagtataacgagaaggcatcttcttgttattaaagaatttcgataattgatggggatgcgttccattagtttgttggcggggtaacggcccaccaagacatcgatggataggggttctgagaggaaggtcccccacattggaactgagacacggaccaaactcatacgggaggcagcagtgaggaatattggtcaatggacgagngnnnnaaccagccaagtagcgtgaaggatgactgccctatgggttgtaaacttcttttatatgggaataaagtgcagtatgtatactgttttgtatgtaccatatgaataaggatcggctaactccgtgccagcagccgcggtaatacggaggatccgagcgttatccggatttattgggtttaaagggagcgtaggcggaagcttaagtcagttgtgaaagtttgcggctcaaccgtaaaattgcagttgatactgggtttcttgagtgcagtagaggtaggcggaattcgtggtgtagcggtgaaatgcttagatatcacgaagaactccgattgcgaaggcagcttactggactgtaactgacgctgatgctcgaaagtgtgggtatcaaacaggattagataccctggtagtccacacagtaaacgatgaatactcgctgtttgcgatatacagcaagcggccaagcgaaagcattaagtattccacctggggagtacgccggcaacggtgaaactcaaaggaattgacgggggcccgcacaagcggaggaacatgtggtttaattcgatgatacgcgaggaaccttacccgggcttaaattgcaactgacgtaggtggaaacatctattccttcgggcagttgtgaaggtgctgcatggttgtcgtcagctcgtgccgtgaggtgtcggcttaagtgccataacgagcgcaacccttatctttagttactaacaggttatgctgaggactctagagagactgccgtcgtaagatgtgaggaaggtggggatgacgtcaaatcagcacggcccttacgtccggggctacacacgtgttacaatggggggtacagaaggcagctacacggcgacgtgatgctaatccccaaaacctctctcagttcggattggagtctgcaacccgactccatgaagctggattcgctagtaatcgcgcatcagccacggcgcggtgaatacgttcccgggccttgtacacaccgcccgtcaagccatgaaagccgggggtacctgaagtccgtaaccgtaagna

SEQ ID NO: 38 - DNA, 1384 bp, synthetic construct
  misc_feature 1..1384: note = Synthetic: FBI00060
  source 1..1384: mol_type = other DNA
tgcagtcgaacgggatccatcaggctttgcttggtggtgagagtggcgaacgggtgagtaatgcgtgaccgacctgccccatacaccggaatagctcctggaaacgggtggtaatgccggatgctccagttgatcgcatggtcttctgggaaagctttcgcggtatgggatggggtcgcgtcctatcagcttgacggcggggtaacggcccaccgtggcttcgacgggtagccggcctgagagggcgaccggccacattgggactgagatacggcccagactcctacgggaggcagcagtggggaatattgcacaatgggcgcaagcctgatgcagcgacgccgcgtgagggatggaggccttcgggttgtaaacctcttttatcggggagcaagcgagagtgagtttacccgttgaataagcaccggctaactacgtgccagcagccgcggtaatacgtagggtgcaagcgttatccggaattattgggcgtaaagggctcgtaggcggttcgtcgcgtccggtgtgaaagtccatcgcttaacggtggatccgcgccgggtacgggcgggcttgagtgcggtaggggagactggaattcccggtgtaacggtggaatgtgtagatatcgggaagaacaccaatggcgaaggcaggtctctgggccgttactgacgctgaggagcgaaagcgtggggagcgaacaggattagataccctggtagtccacgccgtaaacggtggatgctggatgtggggcccgttccacgggttccgtgtcggagctaacgcgttaagcatcccgcctggggagtacggccgcaaggctaaaactcaaagaaattgacgggggcccgcacaagcggcggagcatgcggattaattcgatgcaacgcgaagaaccttacctgggcttgacatgttcccgacggtcgtagagatacggcttcccttcggggcgggttcacaggtggtgcatggtcgtcgtcagctcgtgtcgtgagatgttgggttaagtcccgcaacgagcgcaaccctcgccccgtgttgccagcggattatgccgggaactcacgggggaccgccggggttaactcggaggaaggtggggatgacgtcagatcatcatgccccttacgtccagggcttcacgcatgctacaatggccggtacaacgggatgcgacgcggcgacgcggagcggatccctgaaaaccggtctcagttcggatcgcagtctgcaactcgactgcgtgaaggcggagtcgctagtaatcgcgaatcagcaacgtcgcggtgaatgcgttcccgggccttgtacacaccgcccgtcaagtcatgaaagtgggcagcacccgaagccggtggcctaaccccttgtggga

SEQ ID NO: 39 - DNA, 1361 bp, synthetic construct
  misc_feature 1..1361: note = Synthetic: FBI00061
  source 1..1361: mol_type = other DNA
agcantgcngggtggcgaccggcgcacgggtgcgtaacgcgtatgcaacctacccataacagggggataacactgagaaattggtactaataccccataacatcagaaccggcatcggttttggttgagaactccggtggttatggatgggcatgcgttgtattagctggttggtgaggtaacggctcaccaaggcaacgatacatagggggactgagaggttaaccccccacattggtactgagacacganccaactnnctacgggaggcagcagtgaggaatattggtcaatggacgcaagtctgaaccagccatgccgcgtgcaggaagacggctctatgagttgtaaactgcttttgtacgagggtaaacgcttctacgtgtaggagcctgaaagtatcgtacgaataaggatcggctaactccgtgccagcagccgcggtaatacggaggatccaagcgttatccggatttattgggtttaaagggtgcgtaggcggtttgataagttagaggtgaaataccggtgcttaacaccggaactgcctctaatactgttgaactagagagtagttgcggtaggcggaatgtatggtgtagcggtgaaatgcttagagatcatacagaacaccgattgcgaaggcagcttaccaaactatatctgacgttgaggcacgaaagcgtggggagcaaacaggattagataccctggtagtccacgcagtaaacgatgataactcgctgtcggcgatacacagtcggcggctaagcgaaagcgataagttatccacctggggagtacgttcgcaagaatgaaactcaaaggaattgacgggggcccgcacaagcggaggaacatgtggtttaattcgatgatacgcgaggaaccttacccgggcttgaaagttactgacgattctggaaacaggatttcccttcggggcaggaaactaggtgctgcatggttgtcgtcagctcgtgccgtgaggtgtcgggttaagtcccataacgagcgcaacccctaccgttagttgccatcaggtcaagctgggcactctggcgggactgccggtgtaagccgagaggaaggtggggatgacgtcaaatcagcacggcccttacgtccggggctacacacgtgttacaatggtaggtacagagggcagctacccagtgatgggatgcgaatctcgaaagcctatctcagttcggatcggaggctgaaacccgcctccgtgaagttggattcgctagtaatcgcgcatcagccatggcgcggtgaatacgttcccgggccttgtacacaccgcccgtcaagccatggaagctgggggtgcctgaagttcgtgaccgcaagna

SEQ ID NO: 40 - DNA, 1356 bp, synthetic construct
  misc_feature 1..1356: note = Synthetic: FBI00062
  source 1..1356: mol_type = other DNA
cggcacctatcttcggatggaagcgagtggcgaacggctgagtaacacgtggagaacctgccccctcccccgggatagccgcccgaaaggacgggtaataccggataccccggggtgccgcatggcaccccggctaaagccccgacgggaggggatggctccgcggcccatcaggtagacggcggggtgacggcccaccgtgccgacaacgggtagccgggttgagagaccgaccggccagattgggactgagacacggcccagactcctacgggaggcagcagtggggaatcttgcgcaatggggggaaccctgacgcagcgacgccgcgtgcgggacggaggccttcgggtcgtaaaccgctttcagcagggaagagtcaagactgtacctgcagaagaagccccggctaactacgtgccagcagccgcggtaatacgtagggggcgagcgttatccggattcattgggcgtaaagcgcgcgtaggcggcccggcaggccgggggtcgaagcggggggctcaaccccccgaagcccccggaacctccgcggcttgggtccggtaggggagggtggaacacccggtgtagcggtggaatgcgcagatatcgggtggaacaccggtggcgaaggcggccctctgggccgagaccgacgctgaggcgcgaaagctgggggagcgaacaggattagataccctggtagtcccagccgtaaacgatggacgctaggtgtggggggacgatccccccgtgccgcagccaacgcattaagcgtcccgcctggggagtacggccgcaaggctaaaactcaaaggaattgacgggggcccgcacaagcagcggagcatgtggcttaattcgaagcaacgcgaagaaccttaccagggcttgacatatgggtgaagcgggggagaccccgtggccgagaggagcccatacaggtggtggcatggctgtcgtcagctcgtgtcgtgagatgttgggttaagtcccgcaacgagcgcaacccccgccgcgtgttgccatcgggtgatgccgggaacccacgcgggaccgccgccgtcaaggcggaggagggcggggacgacgtcaagtcatcatgccccttatgccctgggctgcacacgtgctacaatggccggtacagagggatgccaccccgcgagggggagcggatcccggaaagccggccccagttcggattgggggctgcaacccgcccccatgaagtcggagttgctagtaatcgcggatcagcatgccgcggtgaatgcgttcccgggccttgtacacaccgcccgtcacaccacccgagtcgtctgcacccgaagtcgcncggcccaaccnnnaggg

SEQ ID NO: 41 - DNA, 1394 bp, synthetic construct
  misc_feature 1..1394: note = Synthetic: FBI00066
  source 1..1394: mol_type = other DNA
ctnncnntgcagtcgtacnggtaaccaagagaagcttgcttctctccggcgacgagtggcgaacgggtgagtaatacatcggaacgtgtccgctcgtgggggacaaccagccgaaaggttggctaataccgcatgagttctacggaagaaagagggggacccgcaagggcctctcgcgagcggagcggccgatgactgattagccggttggtgaggtaacggctcaccaaagcaacgatcagtagctggtctgagaggacgaccagccacactgggactgagacacggcccagactcctacgggaggcagcagtggggaattttggacaatgggcgcaagcctgatccagctattccgcgtgtgggatgaaggccctcgggttgtaaaccacttttgtagagaacgaaaagacaccttttaaaaaagggtgttgctgacggtactctaagaataagcaccggctaactacgtgccagcagccgcggtaatacgtagggtgcgagcgttaatcggaattactgggcgtaaagggtgcgcaggcggttgagtaagacagatgtgaaatccccgagcttaactcgggaatggcatatgtgactgctcgactagagtgtgtcagagggaggtggaattccacgtgtagcagtgaaatgcgtagatatgtggaagaacaccgatggcgaaggcagcctcctgggacataactgacgctcaggcacgaaagcgtggggagcaaacaggattagataccctggtagtccacgccctaaacgatgttaactagttgttgggaagtaaaattctcagtaacgcagccaacgcgagaagttaaccgcctgggaagtacggtcgcaagactaaaactcaaaggaattgacggggacccgcacaagcggtggatgatgtggattaattcgatgcaacgcgaaaaaccttacctacccttgacatgtcaggaagctcttgtaatgagagcgtgcccgcaagggagcctgaacacaggtgctgcatggctgtcgtcagctcgtgtcgtgagatgttgggttaagtcccgcaacgagcgcaacccttgtcactagttgctacgaaagggcactctagtgagactgccggtgacaaaccggaggaaggtggggatgacgtcaagtcctcatggcccttatgggtagggcttcacacgtcatacaatggtcggaacagagggcagcgaagtcgtgagacggagccaatcccagaaaaccgatcgtagtccggattgcagtctgcaactcgactgcatgaagtcggaatcgctagtaatcgcggatcagcatgccgcggtgaatacgttcccgggtcttgtacacaccgcccgtcaaacaatgggagtggtgtttaccagaagtnnntnnga

SEQ ID NO: 42 - DNA, 1375 bp, synthetic construct
  misc_feature 1..1375: note = Synthetic: FBI00067
  source 1..1375: mol_type = other DNA
tcgaacggcagcatgggggcaaccctgatggcgagtggcgaacgggtgagtaatatatcggaacgtacccaagagtgggggacaaccagtcgaaagattggctaataccgcatacgatctaaggatgaaagtgggggaccttcgggcctcacgctcctggagcggccgatagctgattagctagttggtggggtaaaggcctaccaaggcgatgatcagtagctggtctgagaggacgatcagccacactgggactgagacacggcccagactcctacgggaggcagcagtggggaattttggacaatgggggaaaccctgatccagcaatgccgcgtgagtgaagaaggccttcgggttgtaaagctcttttgtcagggaagaaattgattgggctaatatcccgattagatgacggtacctgaagaataagcaccggctaactacgtgccagcagccgcggtaatacgtagggtgcgagcgttaattcggaattactgggcgtaaagcgtgcgcaggcggttgtgtaagacagatgtgaaatgcccgggctcaacctgggaattgcatttgtgactgcacggctagagtgtgtcagaggggggtagaattccacgtgtagcagtgaaatgcgtagatatgtggaggaataccgatggcgaaggcagccccctgggataacactgacgctcatgcacgaaagcgtggggagcaaacaggattagataccctggtagtccacgccctaaacgatgtcgactagttgttgggtcttaattgacttagtaacgcagctaacgcgtgaagtcgaccgcctggggagtacggtcgcaagattaaaactcaaaggaattgacggggacccgcacaagcggtggatgatgtggattaattcgatgcaacgcgaaaaaccttacctacccttgacatgtcaggaatcccgaagagatttgggagtgctcgaaagagagcctgaacacaggtgctgcatggctgtcgtcagctcgtgtcgtgagatgttgggttaagtcccgcaacgagcgcaacccttgtcattagttgctacgaaagggcactctaatgaaactgccggtgacaaaccggaggaaggtggggatgacgtcaagtcctcatggcccttatgggtagggcttcacacgtcatacaatggtgcatacagagggttgccaacccgagagggggagctaatctcagaaagtgcatcgtagtccggattgaagtctgcaactcgacttcatgaagttggaatcgctagtaatcgcggatcagcatgtcgcggtgaatacgttcccgggtcttgtacccaccccccgtcacaccatgggagcgggttttnccagaagtaggtagntta

SEQ ID NO: 43 - DNA, 1380 bp, synthetic construct
  misc_feature 1..1380: note = Synthetic: FBI00068
  source 1..1380: mol_type = other DNA
tgcangtcgaacgagagaattgctagcttgctaataattctctagtggcgcacgggtgagtaacacgtgagtaacctgcccccgagagcgggatagccctgggaaactgggattaataccgcatagtatcgaaagattaaagcagcaatgcgcttggggatgggctcgcggcctattagttagttggtgaggtaacggctcaccaaggcgatgacgggtagccggtctgagaggatgtccggccacactggaactgagacacggtccagacacctacgggtggcagcagtcgagaatcattcacaatgggggaaaccctgatggtgcgacgccgcgtgggggaatgaaggtcttcggattgtaaacccctgtcatgtgggagcaaattaaaaagatagtaccacaagaggaagagacggctaactctgtgccagcagccgcggtaatacagaggtctcaagcgttgttcggaatcactgggcgtaaagcgtgcgtaggctgtttcgtaagtcgtgtgtgaaaggcgcgggctcaacccgcggacggcacatgatactgcgagactagagtaatggagggggaaccggaattctcggtgtagcagtgaaatgcgtagatntcgagaggaacactcgtggcgaaggcgggttcctggacattaactgacgctgaggcacgaaggccaggggagcgaaagggattagatacccctgtagtcctggcagtaaacggtgcacgcttggtgtgcggggaatcgaccccctgcgtgccggagctaacgcgttaagcgtgccgcctggggagtacggtcgcaagattaaaactcaaagaaattgacggggacccgcacaagcggtggagtatgtggcttaattcgatgcaacgcgaagaaccttacctgggcttgacatgtaatgaacaacatgtgaaagcatgcgactcttcggaggcgttacacaggtgctgcatggccgtcgtcagctcgtgtcgtgagatgtttggttaagtccagcaacgagcgcaacccctgttgccagttaccagcacgtgaaggtggggactctggcgagactgcccagatcaactgggaggaaggtggggacgacgtcaggtcagtatggcccttatgcccagggctgcacacgtactacaatgcccagtacagagggggccgaagccgcgaggcggaggaaatcctgaaaactgggcccagttcggactgtaggctgcaacccgcctacacgaagccggaatcgctagtaatggcgcatcagctacggcgccgtgaatacgttcccgggtcttgtacacaccgcccgtcacatcatggaagccggtcgcacccgnaagtatnnnnagccaaccgcaaggaggcannnc

SEQ ID NO: 44 - DNA, 1394 bp, synthetic construct
  misc_feature 1..1394: note = Synthetic: FBI00069
  source 1..1394: mol_type = other DNA
tgcngtcgaacggaactgttttgaaagatttcttcggaatgaatttgatttagtttagtggcggacgggtgagtaacgcgtgagtaacctgccttcaagagggggataacattctgaaaagaatgctaataccgcatgacatatcggaaccacatggttttgatatcaaagattttatcgcttgaagatggactcgcgtccgattagttagttggtgaggtaacggctcaccaagaccgcgatcggtagccggactgagaggttgaacggccacattgggactgagacacggcccagactcctacgggaggcagcagtgggggatattgcgcaatgggggcaaccctgacgcagcaacgccgcgtgaaggatgaaggttttcggattgtaaacttcttttattaaggacgaaaattgacggtacttaatgaataagctccggctaactacgtgccagcagccgcggtaatacgtagggagcaagcgttgtccggatttactgggtgtaaagggtgcgtaggcggctttgcaagtcagatgtgaaatctatgggctcaacccataaactgcatttgaaactgtagagcttgagtgaagtagaggcaggcggaattccccgtgtagcggtgaaatgcgtagagatggggaggaacaccagtggcgaaggcggcctgctgggctttaactgacgctgaggcacgaaagcgtgggtagcaaacaggattagataccctggtagtccacgctgtaaacgatgattactaggtgtggggggtctgaccccttccgtgccggagttaacacaataagtaatccacctggggagtacggccgcaaggttgaaactcaaaggaattgacgggggcccgcacaagcagtggagtatgtggtttaattcgaagcaacgcgaagaaccttaccaggtcttgacatccaactaacgaagtagagatacattaggtgcccttcggggaaagttgagacaggtggtgcatggttgtcgtcagctcgtgtcgtgagatgttgggttaagtcccgcaacgagcgcaacccttgctattagttgctacgcaagagcactctaataggactgccgttgacaaaacggaggaaggtggggacgacgtcaaatcatcatgccccttatgacctgggctacacacgtactacaatggatgttaacagagggaagcaagacagcgatgtggagcaaacccctaaaaacattctcagttcagattgcaggctgcaacccgcctgcatgaagatggaattgctagtaatcgcggatcagcatgccgcggtgaatacgttcccgggccttgtacacaccgcccgtcacaccatgggagccggtaatacccgaagtcagtagtccaacctcgtgaggacgc

SEQ ID NO: 45 - DNA, 1392 bp, synthetic construct
  misc_feature 1..1392: note = Synthetic: FBI00070
  source 1..1392: mol_type = other DNA
tgcagtcgaggggcagcattttagtttgcttgcaaactgaagatggcgaccggcgcacgggtgagtaacacgtatccaacctgccgataactccggaatagcctttcgaaagaaagattaataccggatggtatacgaatatcgcatgatatttttattaaagaatttcggttatcgatggggatgcgttccattagtttgttggcggggtaacggcccaccaagactacgatggataggggttctgagaggaaggtcccccacattggaactgagacacggtccaaactcctacgggaggcagcagtgaggaatattggtcaatgggcgagagcctgaaccagccaagtagcgtgaaggatgaaggctctatgggtcgtaaacttcttttatatgggaataaagttttccacgtgtggaattttgtatgtaccatatgaataaggatcggctaactccgtgccagcagccgcggtaatacggaggatccgagcgttatccggatttattgggtttaaagggagcgtaggtggattgttaagtcagttgtgaaagtttgcggctcaaccgtaaaattgcagttgaaactggcagtcttgagtacagtagaggtgggcggaattcgtggtgtagcggtgaaatgcttagatatcacgaagaactccgattgcgaaggcagctcactagactgttactgacactgatgctcgaaagtgtgggtatcaaacaggattagataccctggtagtccacacagtaaacgatgaatactcgctgtttgcgatatacagtaagcggccaagcgaaagcattaagtattccacctggggagtacgccggcaacggtgaaactcaaaggaattgacgggggcccgcacaagcggaggaacatgtggtttaattcgatgatacgcgaggaaccttacccgggcttaaattgcaacagaatatattggaaacagtatagccgtaaggctgttgtgaaggtgctgcatggttgtcgtcagctcgtgccgtgaggtgtcggcttaagtgccataacgagcgcaacccttatctttagttactaacaggtatgctgaggactctagagagactgccgtcgtaagatgtgaggaaggtggggatgacgtcaaatcagcacggcccttacgtccggggctacacacgtgttacaatggggggtacagaaggcggctacctggtgacaggatgctaatcccaaaaacctctctcagttcggatcgaagtctgcaacccgacttcgtgaagctggattcgctagtaatcgcgcatcagccatggcgcggtgaatacgttcccgggccttgtacacaccgcccgtcaagccatgaaagccgggggtacctgaagtacgtaaccgcaaggagc

SEQ ID NO: 46 - DNA, 1394 bp, synthetic construct
  misc_feature 1..1394: note = Synthetic: FBI00071
  source 1..1394: mol_type = other DNA
tgcagtcgnncgaagcactttattacaatttcttcggaatgacgatttagtgactgagtggcggacgggtgagtaacgcgtgggtaacctgccttatacagggggataacagttagaaatgactgctaataccgcataagcgcacaggattgcatgatctggtgtgaaaaactccggtggtataagatggacccgcgtctgattagctggttggtgaggtaacggcccaccaaggcgacgatcagtagccgacctgagagggtgaccggccacattgggactgagacacggcccaaactcctacgggaggcagcagtggggaatattgcacaatgggggaaaccctgatgcagcgacgccgcgtgagcgaagaagtatttcggtatgtaaagctctatcagcagggaagaaaaaaatgacggtacctgactaagaagccccggctaactacgtgccagcagcccggtaatacgtagggggcaagcgttatccggatttactgggtgtaaagggagcgcaggcggtgcggcaagtctgatgtgaaagcccggggctcaaccccgggactgcattggaaactgtcgtacttgagtatcggagaggtaagtggaattcctagtgtagcggtgaaatgcgtagatattaggaggaacaccagtggcgaaggcggcttactggacgataactgacgctgaggctcgaaagcgtggggagcaaacaggattagataccctggtagtccacgccgtaaacgatgaatactaggtgtcgggggacatagtccttcggtgccgcagcaaacgcaataagtattccacctgggggagtacgttcgcaagaatgaaactcaaaggaattgacggggacccgcacaagcggtggagcatgtggtttaattcgaagcaacgcgaagaaccttaccaagtcttgacatcccggtgacaaagtatgtaatgtactctttcttcggaacaccggtgacaggtggtgcatggttgtcgtcagctcgtgtcgtgagatgttgggttaagtcccgcaacgagcgcaacccctgttcttagtagccagcggttcggccgggcactctagggagactgccagggataacctggaggaaggcggggatgacgtcaaatcatcatgccccttatgacttgggctacacacgtgctacaatggcgtaaacaaagggaagcgaaaaggtgacttctagcaaatcccaaaaataacgtcccagttcggactgtagtctgcaactcgactacacgaagctggaatcgctagtaatcgcgaatcagaatgtcgcggtgaatacgttcccgggtcttgtacacaccgcccgtcacaccatgggagtcgggaatgcccgaagccggtgactcaancgaaaggag

SEQ ID NO: 47 - DNA, 1391 bp, synthetic construct
  misc_feature 1..1391: note = Synthetic: FBI00075
  source 1..1391: mol_type = other DNA
tgcagtcgaggggcagcatggacccagctttgctgggtttgatggcgaccggcgcacgggtgagtaacgcgtatccaacctgccctttactccgggatagtctcctgaaagggagtttaataccggatgtgtttgtctttccgcatgggagcgacaaataaagattaattggtaaaggatggggatgcgtcccattagcttgttggcggggtaacggcccaccaaggcgacgatgggtaggggttctgagaggaaggtcccccacattggaactgagacacggtccaaactcctacgggaggcagcagtgaggaatattggtcaatgggcgggagcctgaaccagccaagtagcgtgaaggacgacggccctacgggttgtaaacttcttttataagggaataaagttcgccacgtgtggtgttttgtatgtaccttatgaataagcatcggctaattccgtgccagcagccgcggtaatacggaagatgcgagcgttatccggatttattgggtttaaagggagcgtaggcgggcttttaagtcagcggtcaaatgtcgtggctcaaccatgtcaagccgttgaaactgtaagccttgagtctgcacagggcacatggaattcgtggtgtagcggtgaaatgcttagatatcacgaagaactccgatcgcgaaggcattgtgccggggcataactgacgctgaggctcgaaagtgcgggtatcaaacaggattagataccctggtagtccgcacggtaaacgatgaatgctcgctatgggcgatatattgtccgtggccaagcgaaagcgttaagcattccacctggggagtacgccggcaacggtgaaactcaaaggaattgacgggggcccgcacaagcggaggaacatgtggtttaattcgatgatacgcgaggaaccttacccgggcttgaattgcaggtgcatgagtcagagatgattctttccttcgggactcctgtgaaggtgctgcatggttgtcgtcagctcgtgccgtgaggtgtcggcttaagtgccataacgagcgcaacccttctccccagttgccatcgggtaatgccgggccctctggggacactgccatcgtaagatgcgaggaaggtggggatgacgtcaaatcagcacggcccttacgtccggggccacacacgtgttacaatggggggtacagagggccgctgtccggtgacggtcggccaatccctaaaacccctctcagttcggactggagtctgcaacccgactccacgaagctggattcgctagtaatcgcgcatcagccatggcgcggtgaatacgttcccgggccttgtacacaccgcccgtcaagccatgaaagccgggggtgcctgaagtccgtgaccgcgag

SEQ ID NO: 48 - DNA, 1525 bp, synthetic construct
  misc_feature 1..1525: note = Synthetic: FBI00076
  source 1..1525: mol_type = other DNA
tggagagtttgatcctggctcaggatgaacgctagctacaggcttaacacatgcaagtcgaggggcagcatttcagtttgcttgcaaactggagatggcgaccggcgcacgggtgagtaacacgtatccaacctgccgataactcggggatagcctttcgaaagaaagattaatacccgatggtataatyagaccgcatggtcttgttattaaagaatttcggttatcgatggggatgcgttccattaggcagttggtgaggtaacggctcaccaaaccttcgatggataggggttctgagaggaaggtcccccacattggaactgagacacggtccaaactcctacgggaggcagcagtgaggaatattggtcaatgggcgcaggcctgaaccagccaagtagcgtgaaggatgactgccctatgggttgtaaacttcttttatatgggaataaagttttccacgtgtggaattttgtatgtaccatatgaataaggatcggctaactccgtgccagcagccgcggtaatacggaggatccgagcgttatccggatttattgggtttaaagggagcgtaggtggacagttaagtcagttgtgaaagtttgcggctcaaccgtaaaattgcagttgatactggctgtcttgagtacagtagaggtgggcggaattcgtggtgtagcggtgaaatgcttagatatcacgaagaactccgattgcgaaggcagctcactggactgcaactgacactgatgctcgaaagtgtgggtatcaaacaggattagataccctggtagtccacacagtaaacgatgaatactcgctgtttgcgatatacagtaagcggccaagcgaaagcattaagtattccacctggggagtacgccggcaacggtgaaactcaaaggaattgacgggggcccgcacaagcggaggaacatgtggtttaattcgatgatacgcgaggaaccttacccgggcttaaattgcatttgaatatattggaaacagtatagccgtaaggcaaatgtgaaggtgctgcatggttgtcgtcagctcgtgccgtgaggtgtcggcttaagtgccataacgagcgcaacccttatctttagttactaacaggtcatgctgaggactctagagagactgccggtcgtaagatgtgaggaaggtggggatgacgtcaaatcagcacggcccttacgtccggggctacacacgtgttacaatggggggtacagaaggcagctacctggtgacaggatgctaatcccaaaagcctctctcagttcggatcgaagtctgcaacccgacttcgtgaagctggattcgctagtaatcgcgcatcagccatggcgcggtgaatacgttcccgggccttgtacacaccgcccgtcaagccatgaaagccgggggtacctgaagtacgtaaccgcaaggagcgtcctagggtaaaactggtaattggggctaagtcgtaacaaggtagccgtaccggaaggtgcggytggatcacctcctt

SEQ ID NO: 49 - DNA, 1397 bp, synthetic construct
  misc_feature 1..1397: note = Synthetic: FBI00077
  source 1..1397: mol_type = other DNA
gcaannnnaacggcagcgcggggagcttgctccctggcggcgagtggcgcacgggtgagtaatacatcggaacgtgtcttctagtgggggataactgcccgaaagggcagctaataccgcatgagacctgagggtgaaagcgggggatcgcaagacctcgcgctggaagagcggccgatgtccgattagctagttggtgaggtaaaggctcaccaaggcgacgatcggtagctggtctgagaggacgaccagccacactgggactgagacacggcccagactcctacgggaggcagcagtggggaattttggacaatgggggcaaccctgatccagccatgccgcgtgcaggatgaaggtcttcggattgtaaactgcttttgtcagggacgaaaagggatgcgataacaccgcattccgctgacggtacctgaagaataagcaccggctaactacgtgccagcagccgcggtaatacgtagggtgcaagcgttaatcggaattactgggcgtaaagcgtgcgcaggcggttctgtaagatagatgtgaaatccccgggctcaacctgggaattgcatatatgactgcaggacttgagtttgtcagaggagggtggaattccacgtgtagcagtgaaatgcgtagatatgtggaagaacaccgatggcgaaggcagccctctgggacatgactgacgctcatgcacgaaagcgtggggagcaaacaggattagataccctggtagtccacgccctaaacgatgtctactagttgttggggacgatagtcnttggtaacgcagctaacgcgtgaagtagaccgcctggggagtacggtcgcaagattaaaactcaaaggaattgacggggacccgcacaagcggtggatgatgtggattaattcgatgcaacgcgaaaaaccttacctagccttgacatgccaggaaggcctgagagatcaggccgtgcccgcaagggaatctggacacaggtgctgcatggctgtcgtcagctcgtgtcgtgagatgttgggttaagtcccgcaacgagcgcaacccttgtcattagttgctacgaaagggcactctaatgagactgccggtgacaaaccggaggaaggtggggatgacgtcaagtcctcatggcccttatggctagggcctcacacgtcatacaatggtcggaacagagggaagcgaagccgcgaggtgaagccaatcccagaaaaccgatcgtagtccggattgcagtctgcaactcgactgcatgaagtcggaatcgctagtaatcgcggatcagcatgccgcggtgaatacgttcccgggtcttgtacacaccgcccgtcacaccatgggagtggggttcaccagaagacgtttgcccaaccgaaaggaaggc

SEQ ID NO: 50 - DNA, 1393 bp, synthetic construct
  misc_feature 1..1393: note = Synthetic: FBI00078
  source 1..1393: mol_type = other DNA
tacnntgcagtcgnacgggaaaccttttattgaagcttcggcagatttggctggtttctagtggcggacgggtgagtaacgcgtgggtaacctgccttatacagggggataacaaccagaaatggttgctaataccgcataagcgcacaggaccgcatggtccggtgtgaaaaactccggtggtataagatggacccgcgttggattagctagttggcagggtaacggcctaccaaggcgacgatccatagccggcctgagagggtgaacggccacattgggactgagacacggcccagactcctacgggaggcagcagtggggaatattgcacaatgggggaaaccctgatgcagcgacgccgcgtgaaggaagaagtatctcggtatgtaaacttctatcagcagggaagatagtgacggtacctgactaagaagccccggctaactacgtgccagcagccgcggtaatacgtagggggcaagcgttatccggatttactgggtgtaaagggagcgtagacggactggcaagtctgatgtgaaaggcgggggctcaacccctggactgcattggaaactgttagtcttgagtgccggagaggtaagcggaattcctagtgtagcggtgaaatgcgtagatattaggaggaacaccagtggcgaaggcggcttactggacggtaactgacgttgaggctcgaaagcgtggggagcaaacaggattagataccctggtagtccacgccgtaaacgatgaatactaggtgttggggagcaaagctcttcggtgccgccgcaaacgcattaagtattccacctggggagtacgttcgcaagaatgaaactcaaaggaattgacggggacccgcacaagcggtggagcatgtggtttaattcgaagcaacgcgaagaaccttaccaagtcttgacatccctctgaccnnnccttaaccgantctttccttcgggacaggggagacaggtggtgcatggttgtcgtcagctcgtgtcgtgagatgttgggttaagtcccgcaacgagcgcaacccctatccccagtagccagcggttcggccgggcactctgaggagactgccagggataacctggaggaaggcggggatgacgtcaaatcatcatgccccttatgatttgggctacacacgtgctacaatggcgtaaacaaagggaagcaagcctgcgaaggtaagcaaatcccaaaaataacgtcccagttcggactgcagtctgcaactcgactgcacgaagctggaatcgctagtaatcgcggatcagaatgccgcggtgaatacgttcccgggtcttgtacacaccgcccgtcacaccatgggagtcagtaacgcccgaagtcagtgacctaactgcaaaga

SEQ ID NO: 51 - DNA, 1392 bp, synthetic construct
  misc_feature 1..1392: note = Synthetic: FBI00079
  source 1..1392: mol_type = other DNA
tgcagtcgaacgaagcaattaagatgaagttttcggatggaatcttgattgactgagtggcggacgggtgagtaacgcgtggataacctgcctcacactgggggataacagttagaaatgactgctaataccgcataagcgcacagtgccgcatggcagtgtgtgaaaaactccggtggtgtgagatggatccgcgtctgattagccagttggcggggtaacggcccaccaaagcgacgatcagtagccgacctgagagggtgaccggccacattgggactgagacacggcccaaactcctacgggaggcagcagtggggaatattgcacaatgggcgaaagcctgatgcagcgacgccgcgtgagtgaagaagtatttcggtatgtaaagctctatcagcagggaagaaaatgacggtacctgactaagaagccccggctaactacgtgccagcagccgcggtaatacgtagggggcaagcgttatccggatttactgggtgtaaagggagcgtagacggcgaagcaagtctgaagtgaaaacccagggctcaaccctgggactgctttggaaactgttttgctagagtgtcggagaggtaagtggaattcctagtgtagcggtgaaatgcgtagatattaggaggaacaccagtggcgaaggcggcttactggacgataactgacgttgaggctcgaaagcgtggggagcaaacaggattagataccctggtagtccacgccgtaaacgatgaatgctaggtgttggggggcaaagcccttcggtgccgccgcaaacgcagtaagcattccacctggggagtacgttcgcaagaatgaaactcaaaggaattgacggggacccgcacaagcggtggagcatgtggtttaattcgaagcaacgcgaagaaccttaccaagtcttgacatccccctgacgggccggtaacgcggcctttccttcgggacaggggagacaggtggtgcatggttgtcgtcagctcgtgtcgtgagatgttgggttaagtcccgcaacgagcgcaacccttatccttagtagccagcaggtagagccgggcactctagggagactgccagggataacctggaggaaggtggggatgacgtcaaatcatcatgccccttatgatttgggctacacacgtgctacaatggcgtaaacaaagggaagcgagacagtgatgtggagcaaatcccaaaaataacgtcccagttcggactgtagtctgcaacccgactacacgaagctggaatcgctagtaatcgcgaatcagaatgtcgcggtgaatacgttcccgggtcttgtacacaccgcccgtcacaccatgggagtcagcaacgcccgaagtcagtgacccaaccgaaaggagg

SEQ ID NO: 52 - DNA, 1389 bp, synthetic construct
  misc_feature 1..1389: note = Synthetic: FBI00080
  source 1..1389: mol_type = other DNA
cgaacggcagcacagggagcttgctcccgggtggcgagtggcgcacgggtgagtaatacatcggaacgtgtcctgttgtgggggataactgctcgaaagggtggctaataccgcatgagacctgagggtgaaagcgggggatcgcaagacctcgcgcaattggagcggccgatgcccgattagctagttggtgaggtaaaggctcaccaaggcgacgatcggtagctggtctgagaggacgaccagccacactgggactgagacacggcccagactcctacgggaggcagcagtggggaattttggacaatgggggcaaccctgatccagccatgccgcgtgcaggatgaaggccttcgggttgtaaactgcttttgtcaggacgaaaaggaccgtgttaataccatggtctgctgacggtacctgaagaataagcaccggctaactacgtgccagcagccgcggtaatacgtagggtgcaagcgttaatcggaattactgggcgtaaagcgtgcgcaggcggttctgtaagacagatgtgaaatccccgggctcaacctgggaattgcatttgtgactgcaggactagagttcatcagaggggggtggaattccaagtgtagcagtgaaatgcgtagatatttggaagaacaccaatggcgaaggcagccccctgggatgcgactgacgctcatgcacgaaagcgtggggagcaaacaggattagataccctggtagtccacgccctaaacgatgtctactggttgttggggttttttaaccttggtaacgaagctaacgcgtgaagtagaccgcctggggagtacggtcgcaagattaaaactcaaaggaattgacggggacccgcacaagcggtggatgatgtggattaattcgatgcaacgcgaaaaaccttacctagccttgacatgccaggaatcctgaagagattcgggagtgcccgcaagggaatctggacacaggtgctgcatggctgtcgtcagctcgtgtcgtgagatgttgggttaagtcccgcaacgagcgcaacccttgtcactagttgctacgcaagagcactctagtgagactgccggtgacaaaccggaggaaggtggggatgacgtcaagtcctcatggcccttatggctagggcctcacacgtcatacaatggtcggaacagagggcagcgaagccgcgaggtggagcaaatcccagaaaaccgatcgtagtccggattgcagtctgcaactcgactgcatgaagtcggaatcgctagtaatcgcggatcagcatgccgcggtgaatacgttcccgggtcttgtacacaccgcccgtcacaccatgggagtggggttcaccagaagacgtttgtttaaccgcaaggagga

SEQ ID NO: 53 - DNA, 1384 bp, synthetic construct
  misc_feature 1..1384: note = Synthetic: FBI00081
  source 1..1384: mol_type = other DNA
gtcgaggggcagcgagatgtagcaatacgtcgtcggcgaccggcgaatgggtgagtaacacgtatgcaacttacctcttagtggtgaataacccgatgaaagtcggactaatacaccatactctccttagatcacatgagaagaggaggaaagattaatcgctaagagatgggcctgcgttccattagctagttggtaaggtaacggcttaccaaggcaacgatggatagggggactgagaggttgaccccccacattgacactgagatacgggtcaaactcctacgggaggcagcagtgaggaatattggtcaatgggcgagagcctgaaccagccaagtcgcgtgaaggaagactgcccgcaagggttgtaaacttcttttgtgtgggattaaagtcacctacgtgtaggtgtttgcagttaccatacgaataagcatcggctaactccgtgccagcagccgcggtaatacggaggatgcgagcgttatccggaattattgggtttaaagggtgcgtaggttgcaagggaagtcaggggtgaaaagctgtagctcaactatggtcttgcctttgaaactctctagctagagtgtattggaggtacgtggaacgtgtggtgtagcggtgaaatgcatagatatcacacagaactccgattgcgcaggcagcgtactacattacaactgacactgaagcacgaaagcgtgggtatcaaacaggattagataccctggtagtccacgcagtaaacgatgaatactagatctatgcgatacacagtatgggtctaagcgaaagcgataagtattccacctgggggagtacgccggcaacggtgaaactcaaagagattggcgggggtccgcacaagcggaggaacatgtggtttaattcgatgatacgcgaggaaccttacccgggattgaaatgtagatgcatgaggctgagaggtctcttcccttcggggcttctatgtaggtgctgcatggttgtcgtcagctcgtgccgtgaggtgtcggcttaagtgccataacgagcgcaacccgcgtcgatagttactaacgagtcatgtcgaggactctatcgagacagccgtcgtaagacgtgaggaaggagcggatgacgtcaaatcagcacggcccttacatccggggcgacacacgtgttacaatggtagggacagcgagcagccatctggtgacagagagctaatctataaaccctatcccagttcggatcggagtctgcaactcgactctgtgaagctggattcgctagtaatcgcgcatcagccatggcgcggtgaatacgttcccggaccttgcacacaccgcccgtcaagccatgggagtcgggggtacctgaagagcgtgaccgtcac

SEQ ID NO: 54 - DNA, 1394 bp, synthetic construct
  misc_feature 1..1394: note = Synthetic: FBI00085
  source 1..1394: mol_type = other DNA
tgcnntcgaacggaactgttttgaaagatttcttcggaatgaatttgatttagtttagtggcggacgggtgagtaacgcgtgagtaacctgccttcaagagggggataacattctgaaaagaatgctaataccgcatgacatatcggaatcacatgattttgatatcaaagattttatcgcttgaagatggactcgcgtccgattagttagttggtgaggtaacggctcaccaagaccgcgatcggtagccggactgagaggttgaacggccacattgggactgagacacggcccagactcctacgggaggcagcagtgggggatattgcgcaatgggggcaaccctgacgcagcaacgccgcgtgaaggatgaaggttttcggattgtaaacttcttttattaaggacgaaacttgacggtacttaatgaataagctccggctaactacgtgccagcagccgcggtaatacgtagggagcaagcgttgtccggatttactgggtgtaaagggtgcgtaggcggctttgcaagtcagatgtgaaatctatgggctcaacccataaactgcatttgaaactgtagagcttgagtgaagtagaggcaggcggaattccccgtgtagcggtgaaatgcgtagagatggggaggaacaccagtggcgaaggcggcctgctgggctttaactgacgctgaggcacgaaagcgtgggtagcaaacaggattagataccctggtagtccacgctgtaaacgatgattactaggtgtggggggtctgaccccttccgtgccggagttaacacaataagtaatccacctggggagtacggccgcaaggttgaaactcaaaggaattgacgggggcccgcacaagcagtggagtatgtggtttaattcgaagcaacgcgaagaaccttaccaggtcttgacatccaactaacgaagtagagatacattaggtgcccttcggggaaagttgagacaggtggtgcatggttgtcgtcagctcgtgtcgtgagatgttgggttaagtcccgcaacgagcgcaacccttgctattagttgctacgcaagagcactctaataggactgccgttgacaaaacggaggaaggtggggacgacgtcaaatcatcatgccccttatgacctgggctacacacgtactacaatggatgttaacagagggaagcaagacagcgatgtggagcaaacccctaaaaacattctcagttcagattgcaggctgcaacccgcctgcatgaagatggaattgctagtaatcgcggatcagcatgccgcggtgaatacgttcccgggccttgtacacaccgcccgtcacaccatgggagccggtaatacccgaagtcagtagtccaacctcgtgaggacgc

SEQ ID NO: 55 - DNA, 1532 bp, synthetic construct
  misc_feature 1..1532: note = Synthetic: FBI00087
  source 1..1532: mol_type = other DNA
tggagagtttgatcctggctcaggatgaacgctggcggcgtgcctaacacatgcaagtcgaacgaagcgcttccgcctgattttcttcggagatgaaggcggctgcgactgagtggcggacgggtgagtaacgcgtgggcaacctgccttgcactgggggataacagccagaaatggctgctaataccgcataagaccgaagcgccgcatggcgcdgcggccaaagccccggcggtgcaagatgggcccgcgtctgattaggtrgttggcggggtaacggcccaccaagccgacgatcagtagccgacctgagagggtgaccggccacattgggactgagacacggcccagactcctacgggaggcagcagtggggaatattgcacaatgggggaaaccctgatgcagcgacgccgcgtgaaggatgaagtatttcggtatgtaaacttctatcagcagggaagaagatgacggtacctgactaagaagccccggctaactacgtgccagcagccgcggtaatacgtagggggcaagcgttatccggatttactgggtgtaaagggagcgtagacggcgatgcaagccagatgtgaaagcccggggctcaaccccgggactgcatttggaactgcgtggctggagtgtcggagaggcaggcggaattcctagtgtagcggtgaaatgcgtagatattaggaggaacaccagtggcgaaggcggcctgctggacgatgactgacgttgaggctcgaaagcgtggggagcaaacaggattagataccctggtagtccacgccgtaaacgatgactactaggtgtcgggtggcaaggccattcggtgccgcagcaaacgcaataagtagtccacctggggagtacgttcgcaagaatgaaactcaaaggaattgacggggacccgcacaagcggtggagcatgtggtttaattcgaagcaacgcgaagaaccttacctgatcttgacatcccgatgccaaagcgcgtaacgcgctctttcttcggaacatcggtgacaggtggtgcatggttgtcgtcagctcgtgtcgtgagatgttgggttaagtcccgcaacgagcgcaacccctatcttcagtagccagcatttyggatgggcactctggagagactgccagggacaacctggaggaaggtggggatgacgtcaaatcatcatgccccttatgaccagggctacacacgtgctacaatggcgtaaacaaagggaggcgaacccgcgagggtgggcaaatcccaaaaataacgtctcagttcggattgtagtctgcaactcgactacatgaagctggaatcgctagtaatcgcgaatcagaatgtcgcggtgaatacgttcccgggtcttgtacacaccgcccgtcacaccatgggagtcagtaacgcccgaagccggtgacccaacccgcaagggagggagccgtcgaaggtgggaccgataactggggtgaagtcgtaacaaggtagccgtatcggaaggtgcggytggatcacctcctt

SEQ ID NO: 56 - DNA, 1389 bp, synthetic construct
  misc_feature 1..1389: note = Synthetic: FBI00092
  source 1..1389: mol_type = other DNA
tgcagtcgagcgagaaatttttaacggattccttcggggagaagataaggatggaaagcggcggacgggtgagtaacgcgtgagtaacctgcctttaggagggggacaacattccgaaagggatgctaataccgcataaaattattgtatcgcatggtatgataatcaaagatttatcgcctaaagatggactcgcgtccgattagctagttggtggggtaaaagcctaccaaggcgacgatcggtagccgaactgagaggttgatcggccacattgggactgagacacggcccagactcctacgggaggcagcagtgggggatattgcgcaatgggggaaaccctgacgcagcaacgccgcgtgaaggaagaaggccttcgggttgtaaacttctttaagtgtggaagataatgacggtacacacagaataagccacggctaactacgtgccagcagccgcggtaatacgtaggtggcaagcgttgtccggatttactgggtgtaaagggcgtgtaggcgggtagacaagtcagatgtgaaataccggggctcaactccggggctgcatttgaaactgtatatcttgagtgtcggagaggaaagcggaattcctagtgtagcggtgaaatgcgtagatattaggaggaacaccagtggcgaaggcggctttctggacgataactgacgctgaggcgcgaaagcgtggggagcaaacaggattagataccctggtagtccacgccgtaaacgatggatactaggtgtaggaggtatcgaccccttctgtgccgcagttaacacaataagtatcccacctggggagtacggtcgcaagattgaaactcaaaggaattgacgggggcccgcacaagcagtggagtatgtggtttaattcgaagcaacgcgaagaaccttaccaggacttgacatcccacgcatagcctagagataggtgaagtcctacgggacgtggagacaggtggtgcatggttgtcgtcagctcgtgtcgtgagatgttgggttaagtcccgcaacgagcgcaacccttactgtcagttaccatcattaagttggggactctggcaggactgccggtgacaaatcggaggaaggtggggacgacgtcaaatcatcatgccccttatgtcctgggctacacacgtactacaatggctgtaaacagagtgaagcaaagcagtgatgtggagcaaaacacaaaaagcagtctcagttcagattgtaggctgaaactcgcctatatgaagtcggaattgctagtaatcgcagatcagcatgctgcggtgaatacgttcccgggccttgtacacaccgcccgtcacaccatgagagtcgataacacccgaagcctgtagcttaaccttaggga

SEQ ID NO: 57 - DNA, 1391 bp, synthetic construct
  misc_feature 1..1391: note = Synthetic: FBI00093
  source 1..1391: mol_type = other DNA
gcaagtcgaacgaagcactttaattgatttcttcggaatgaagtttttgtgactgagtggcggacgggtgagtaacgcgtgggtaacctgcctcatacagggggataacagttggaaacgactgctaataccgcataagcgcacaggattgcatgatccagtgtgaaaaactccggtggtatgagatggacccgcgtctgattagccagttggcggggtaacggcccaccaaagcgacgatcagtagcnnacctgagagggtgaccggccacattgggactgagacacggcccaaactcctacgggaggcagcagtggggaatattgcacaatgggggaaaccctgatgcagcgacgccgcgtgagcgaagaagtatttcggtatgtaaagctctatcagcagggaagaagaatgacggtacctgactaagaagcaccggctaaatacgtgccagcagccgcggtaatacgtatggtgcaagcgttatccggatttactgggtgtaaagggagcgcaggcggtacggcaagtctgatgtgaaatcccggggctcaaccccggtactgcattggaaactgtcggactagagtgtcggaggggtaagtggaattcctagtgtagcggtgaaatgcgtagatattaggaggaacaccagtggcgaaggcggcttactggacgattactgacgctgaggctcgaaagcgtggggagcaaacaggattagataccctggtagtccacgccgtaaacgatgaatactaggtgtcggggagcattgctcttcggtgccgcagcaaacgcaataagtattccacctggggagtacgttcgcaagaatgaaactcaaaggaattgacggggacccgcacaagcggtggagcatgtggtttaattcgaagcaacgcgaagaaccttaccaagtcttgacatcccactgaccaagtatgtaatgtacttttctcttcggagcagtggtgacaggtggtgcatggttgtcgtcagctcgtgtcgtgagatgttgggttaagtcccgcaacgagcgcaacccctattcttagtagccagcggttcggccgggcactctagggagactgccagggataacctggaggaaggtggggatgacgtcaaatcatcatgccccttatgacttgggctacacacgtgctacaatggcgtaaacaaagggaagcaatcccgcgagggggagcaaatctcaaaaataacgtctcagttcggactgtagtctgcaactcgactacacgaagctggaatcgctagtaatcgcgaatcagaatgtcgcggtgaatacgttcccgggtcttgtacacaccgcccgtcacaccatgggagttggtaatgcccgaagtcagtgacccaaccgcaagga

SEQ ID NO: 58 - DNA, 1371 bp, synthetic construct
  misc_feature 1..1371: note = Synthetic: FBI00096
  source 1..1371: mol_type = other DNA
anncntgcagtcgaacgatgaaaccgccctcgggcggacatgaagtggcgaacgggtgagtaacacgtgaccaacctgcccccctctccgggacaaccttgggaaaccgaggctaataccggatactccctcccctgctcctgcaggggtcgggaaagcccaggcggagggggatggggtcgcggcccattaggtagtaggcggggtaacggcccacctagcccgcgatgggtagccgggttgagagaccgaccggccacattgggactgagatacggcccagactcctacgggaggcagcagtggggaattttgcgcaatgggggcaaccctgacgcagcaacgccgcgtgcgggacgacggccttcgggttgtaaaccgctttcagcagggaagaaattcgacggtacctgcagaagaagctccggctaactacgtgccagcagccgcggtaatacgtagggagcgagcgttatccggattcattgggcgtaaagagcgcgtaggcggcctctcaagcgggatctctaatccgagggctcaacccccggccggatcccgaactgggaggctcgagttcggtagaggcaggcggaattcccggtgtagcggtggaatgcgcagatatcgggaagaacaccgatggcgaaggcagcctgctgggccgcaactgacgctgaggcgcgaaagctaggggagcgaacaggattagataccctggtagtcctagccgtaaacgatggatactaggtgtggggggctccgccctccgtgccgcagccaacgcattaagtatcccgcctggggagtacggccgcaaggctaaaactcaaaggaattgacgggggcccgcacaagcagcggagcatgtggcttaattcgaagcaacgcgaagaaccttaccagggcttgacatggacgtgaagccggggaaacccggtggccgagaggagcgtccgcaggtggtgcatggctgtcgtcagctcgtgtcgtgagatgttgggttaagtcccgcaacgagcgcaacccctgccccatgttgccagcattaggttggggactcatgggggactgccggcgtcaagccggaggaaggtggggacgacgtcaagtcatcatgccctttatgccctgggctgcacacgtgctacaatggccggtacaacgggctgcgagaccgcgaggtcgagcgaatccctcaaagccggccccagttcggatcggaggctgcaacccgcctccgtgaagtcggagttgctagtaatcgcggatcagcatgccgcggtgaatacgttcccgggccttgtacacaccgcccgtcacaccacccgagtcgtctgcacccgaagccgccggccgaacccgcaaggg

SEQ ID NO: 59 - DNA, 1384 bp, synthetic construct
  misc_feature 1..1384: note = Synthetic: FBI00097
  source 1..1384: mol_type = other DNA
tgcagtcgaacggagctattttggcggaacccttcggggggaagctggtttagcttagtggcggacgggtgagtaacacgtgagcaacctgcctttcagagggggataacgtttggaaacgaacgctaataccgcataacgcagcgggccggcatcggcctgctgccaaaggagcaatccgctgaaagatgggctcgcgtccgattagatagttggtgaggtaacggcccaccaagtcgacgatcggtagccggactgagaggttgaacggccacattgggactgagacacggcccagactcctacgggaggcagcagtgggggatattgcacaatgggcgaaagcctgatgcagcgacgccgcgtgagggaagacggccttcgggttgtaaacctctgtctttggggacgataatgacggtacccaaggaggaagctccggctaactacgtgccagcagccgcggtaatacgtagggagcgagcgttgtccggaattactgggtgtaaagggagcgtaggcgggagtgcaagttggatgtgaaaaccacgggctcaacccatggactgcattcaaaactgtatttcttgagtgatgtagaggcaggcggaattcctagtgtagcggtgaaatgcgtagatattaggaggaacaccagtggcgaaggcggcctgctgggcatttactgacgctgaggctcgaaagtgtggggagcaaacaggattagataccctggtagtccacaccgtaaacgatgattactaggtgtgggtgcgtcaagcatccgtgccggagttaacacaataagtaatccacctggggagtacgaccgcaaggttgaaactcaaaggaattgacgggggcccgcacaagcagtggagtatgtggtttaattcgaagcaacgcgaagaaccttaccaggtcttgacatcggatgcatagcttagagataagtgaagtccttcgggacatccagacaggtggtgcatggttgtcgtcagctcgtgtcgtgagatgttgggttaagtcccgcaacgagcgcaacccttatgtttagttgctacgcaagagcactctaaagagactgccgttgacaaaacggaggaaggtggggatgacgtcaaatcatcatgccccttatgacctgggccacacacgtactacaatggcgatcaacagagggaagcaaagccgcgaggcagagcaaacccctaaaagtcgtctcagttcggattgcaggctgcaacccgcctgcatgaagccggaattgctagtaatcgcggatcagcatgccgcggtgaatacgttcccgggccttgtacacaccgcccgtcacaccatggaagtcggtaacacccgaagccagtagcccaaccgcaaggag

SEQ ID NO: 60 - DNA, 1379 bp, synthetic construct
  misc_feature 1..1379: note = Synthetic: FBI00099
  source 1..1379: mol_type = other DNA
anncatgcagtcgancgattaagacgccttcgggcgtgcatagagtggcgaacgggtgagtaacacgtgaccaacctgccccctaccccgggataacgcaaggaaacctgcgctaataccggatactccttcccctgctcctgcaggggtcgggaaagcccagacggtaggggatggggtcgcggcccattaggtagacggcggggcaacggcccaccgtgcctgcgatgggtagccgggttgagagaccgaccggccacattgggactgagatacggcccagactcctacgggaggcagcagtggggaattttgcgcaatggggggaaccctgacgcagcaacgccgcgtgcgggatgaaggccttcgggttgtaaaccgctttcagcagggaagatagtgacggtacctgcagaagaagccccggctaactacgtgccagcagccgcggtaatacgtagggggcgagcgttatccggattcattgggcgtaaagcgcgcgtaggcggcccgtcaagcggaacctctaacccgagggctcaacccccggtcgggttccgaactggcaggctcgagtttggtagaggaagatggaattcccggtgtagcggtggaatgcgcagatatcgggaagaacaccgatggcgaaggcagtcttctgggccatcaactgacgctgaggcgcgaaagctgggggagcgaacaggattagataccctggtagtcccagccgtaaacgatgggtgctaggtgtggggggatcatccctccgtgccgcagccaacgcattaagcaccccgcctggggagtacggccgcaaggctaaaactcaaaggaattgacgggggcccgcacaagcagcggagcatgtggcttaattcgaagcaacgcgaagaaccttaccagggcttgacatgctagtgaagccggggaaacccggtggccgagaggagctagcgcaggtggtgcatggctgtcgtcagctcgtgtcgtgagatgttgggttaagtcccgcaacgagcgcaacccctatcgtatgttgccagcattgagttggggactcatacgagactgccggcgtcaagccggaggaaggtggggacgacgtcaagtcatcatgccctttatgccctgggctgcacacgtgctacaatggccggtacaacgggccgcgacctggcgacaggaagcgaatccctcaaagccggccccagttcggatcggaggctgcaacccgcctccgtgaagtcggagttgctagtaatcgcggatcagcatgccgcggtgaatacgttcccgggccttgtacacaccgcccgtcacaccacccgagtcgtctgcacccgaagccgccggccgaacccgcaaggggcggaggc

SEQ ID NO: 61 - DNA, 1408 bp, synthetic construct
  misc_feature 1..1408: note = Synthetic: FBI00102
  source 1..1408: mol_type = other DNA
acncntgcagtcgaacgaagcngatctagcggaagttttcggatggaagttannattgactgagtggcggacgggtgagtaacgcgtgggtaacctgcctcacactgggggacaacagttagaaatgactgctaataccgcataagcgcacaggaccgcatggtccggtgtgaaaaactccggtggtgtgagatggacccgcgtttgattagctagttggtggggtaacggcctaccaaggcgacgatcaatagccgacctgagagggtgaccggccacattgggactgagacacggcccaaactcctacgggaggcagcagtggggaatattgcacaatgggggaaaccctgatgcagcgacgccgcgtgagtgaagaagtatttcggtatgtaaagctctatcagcagggaagaaaatgacggtacctgactaagaagccccggctaactacgtgccagcagccgcggtaatacgtagggggcaagcgttatccggatttactgggtgtaaagggagcgtagacggtaaagcaagtctgaagtgaaagcccggggctcaaccccgggactgctttggaaactgtttaactagagtgctggagaggtaagcggaattcctagtgtagcggtgaaatgcgtagatattaggaggaacaccagtggcgaaggcggcttactggacagtaactgacgttgaggctcgaaagcgtggggagcaaacaggattagataccctggtagtccacgccgtaaacgatgaatactaggtgttgggggacaaagtccttcggtgccgtcgcaaacgcaataagtattccacctggggagtacgttcgcaagaatgaaactcaaaggaattgacggggacccgcacaagcggtggagcatgtggtttaattcgaagcaacgcgaagaaccttaccaaatcttgacatccctctgaccggcgagtaatgtcgcctttccttcgggacagaggagacaggtggtgcatggttgtcgtcagctcgtgtcgtgagatgttgggttaagtcccgcaacgagcgcaacccttatcctaagtagccagcagttcggctgggcactcttgggagactgccagggataacctggaggaaggtggggatgacgtcaaatcatcatgccccttatgatttgggctacacacgtgctacaatggcgtaaacaaagagaagcgagaccgcgaggtggagcaaatctcaaaaataacgtctcagttcggactgcaggctgcaactcgcctgcacgaagctggaatcgctagtaatcgcgaatcagaatgtcgcggtgaatacgttcccgggtcttgtacacaccgcccgtcacaccatgggagtcagtaatgcccgaagtcagtgacccaaccgtaaggagggagctgccga

SEQ ID NO: 62 - DNA, 1390 bp, synthetic construct
  misc_feature 1..1390: note = Synthetic: FBI00104
  source 1..1390: mol_type = other DNA
tgcagtcnnnnggaattatcttattgaaacttcggtcgatttaatttaattctagtggcggacgggtgagtaacgcgtgggtaacctgccttatacagggggataacagtcagaaatgactgctaataccgcataagcgcacagagctgcatggctcagtgtgaaaaactccggtggtataagatggacccgcgttggattagcttgttggtggggtaacggcccaccaaggcgacgatccatagccggcctgagagggtgaacggccacattgggactgagacacggcccagactcctacgggaggcagcagtggggaatattgcacaatgggggaaaccctgatgcagcgacgccgcgtgaaggaagaagtatctcggtatgtaaacttctatcagcagggaagataatgacggtacctgactaagaagccccggctaactacgtgccagcagccgcggtaatacgtagggggcaagcgttatccggatttactgggtgtaaagggagcgtagacggtgtggcaagtctgatgtgaaaggcatgggctcaacccgtggactgcattggaaactgtcatacttgagtgccggaggggtaagcggaattcctagtgtagcggtgaaatgcgtagatattaggaggaacaccagtggcgaaggcggcttactggacggtaactgacgttgaggctcgaaagcgtggggagcaaacaggattagataccctggtagtccacgccgtaaacgatgaatactaggtgtcgggnngcnnnctttcggtgccgtcgcaaacgcaataagtattccacctggggagtacgttcgcaagaatgaaactcaaaggaattgacggggacccgcacaagcggtggagcatgtggtttaattcgaagcaacgcgaagaaccttaccaagtcttgacatccccctgaccggaacttaaccgttcctttccttcgggacaggggagacaggtggtgcatggttgtcgtcagctcgtgtcgtgagatgttgggttaagtcccgcaacgagcgcaacccctatcctcagtagccagcagttcggctgggcactctgtggagactgccagggataacctggaggaaggcggggatgacgtcaaatcatcatgccccttatgatttgggctacacacgtgctacaatggcgtaaacaaagggaagcaagcctgtgaaggtaagcaaatcccaaaaataacgtcccagttcggactgcagtctgcaactcgactgcacgaagctggaatcgctagtaatcgcggatcagaatgccgcggtgaatacgttcccgggtcttgtacacaccgcccgtcacaccatgggagtcagtaacgcccgaagtcagtgacctaaccgcaagggaggag

SEQ ID NO: 63 - DNA, 1177 bp, synthetic construct
  misc_feature 1..1177: note = Synthetic: FBI00109
  source 1..1177: mol_type = other DNA
tgcagtcgaacgaagcacttnnnnntgattcttcggatgangnnnnttgtgactgagtggcggacgggtgagtaacgcgtgggtaacctgcctcatacagggggataacagttagaaatgactgctaataccgcataagaccacggagccgcatggctcggtgggaaaaactccggtggtatgagatggacccgcgtctgattaggtagttggtggggtaacggcctaccaagccaacgatcagtagccgacctgagagggtgaccggccacattgggactgagacacggcccaaactcctacgggaggcagcagtggggaatattgcacaatgggggaaaccctgatgcagcgacgccgcgtgagcgaagaagtatttcggtatgtaaagctctatcagcagggaagaaaatgacggtacctgactaagaagcaccggctaaatacgtgccagcagccgcggtaatacgtatggtgcaagcgttatccggatttactgggtgtaaagggagcgtagacggctgtgtaagtctgaagtgaaagcccggggctcaaccccgggactgctttggaaactatgcagctagagtgtcggagaggtaagtggaattcccagtgtagcggtgaaatgcgtagatattgggaggaacaccagtggcgaanggcggcttactggacgatgactgacgttgaggctcgaaagcgtggggagcaaacaggattagataccctggtagtccacgccgtaaacgatgactactaggtgtcggggagcaaagctcttcggtgccgcagcaaacgcaataagtagtccacctggggagtacgttcgcaagaatgaaactcaaaggaattgacggggacccgcacaagcggtggagcatgtggtttaattcgaagcaacgcgaagaaccttacctgctcttgacatcccggtgaccggcatgtaatgatgccttttcttcggaacaccggtgacaggtggtgcatggttgtcgtcagctcgtgtcgtgagatgttgggttaagtcccgcaacgagcgcaacccttatcttcagtagccagcattttggatgggcactctggagagactgccagggataacctggaggaaggtggggatgacgtcnaatcatcatgcnccttatgagcagggctacacacgtgctacaatggc

SEQ ID NO: 64 - DNA, 1386 bp, synthetic construct
  misc_feature 1..1386: note = Synthetic: FBI00110
  source 1..1386: mol_type = other DNA
tgcagtcgaacgaagcatctataggangttttcggatggaatatgggatgactgagtggcggacgggtgagtaacgcgtggataacctgcctcacactgggggataacagttagaaatggctgctaataccgcaaaagcgcacagtaccgcagggtacggtgtgaaaaacccaggtggtgtgagatggatccgcgtctgattagccagttggcggggtaacggcccaccaaagcgactatagtagccgacctgagagggtgaccggccacattgggactgagacacggcccaaactcctacgggaggcagcagtggggaatattgcacaatgggcgaaagcctgatgcagcgaccccgcgtgagtgaagaagtatctcggtatgtaaagctctatcagcagggaagaaaatgacggtacctgactaanaagccccggctaactacgtgccagcagccgcggtaatacgtagggggcaagcgttatccggatttactgggtgtaaagggagcgtagacggcgatgcaagtctgaagtgaaagcccggggctcaaccccgggactgctttggaaactgtgtggctggagtgcaggagaggtaagtggaattcctagtgtagcggtgaaatgcgtagatattaggaggaacaccagtggcgaaggcggcttactggactgtaactgacgttgaggctcgaaagcgtggggagcaaacaggattagataccctggtagtccacgccgtaaacgatgaatgctaggtgtcggggggcaaagcccctcggtgccgccgctaacgcaataagcattccacctggggagtacgttcgcaagaatgaaactcaaaggaattgacggggacccgcacaagcggtggagcatgtggtttaattcgaagcaacgcgaagaaccttaccaagtcttgacatccccctgaccggacagtaacgtgtcccttccttcgggacaggggagacaggtggtgcatggttgtcgtcagctcgtgtcgtgagatgttgggttaagtcccgcaacgagcgcaacccttatccttagtagccagcatttcgggtgggcactctagggagactgccagggataacctggaggaaggtggggatgacgtcaaatcatcatgccccttatgatttgggctacacacgtgctacaatggcgtaaacaaagggaagcggccctgcgaaggtgagcaaatcccaaaaataacgtcccagttcggactgtagtctgcaacccgactacacgaagctggaatcgctagtaatcgcggatcagaatgccgcggtgaatacgttcccgggtcttgtacacaccgcccgtcacaccatgggagtcagcaacgcccgaagtcagtgacccaancgaaagg

SEQ ID NO: 65 - DNA, 1395 bp, synthetic construct
  misc_feature 1..1395: note = Synthetic: FBI00111
  source 1..1395: mol_type = other DNA
tgcagtcgaggggcagcatggtcttagcttgctaaggccgatggcgaccggcgcacgggtgagtaacacgtatccaacctgccgtctactcttggacagccttctgaaaggaagattaatacaagatggcatcatgagtccacatgttcacatgattaaaggtattccggtagacgatggggatgcgttccattagatagtaggcggggtaacggcccacctagtcttcgatggataggggttctgagaggaaggtcccccacattggaactgagacacggtccaaactcctacgggaggcagcagtgaggaatattggtcaatgggcgcaggcctgaaccagccaagtagcgtgaaggatgactgccctatgggttgtaaacttcttttataaaggaataaagtcgggtatgcatacccgtttgcatgtactttatgaataaggatcggctaactccgtgccagcagccgcggtaatacggaggatccgagcgttatccggatttattgggtttaaagggagcgtagatggatgtttaagtcagttgtgaaagtttgcggctcaaccgtaaaattgcagttgatactggatatcttgagtgcagttgaggcaggcggaatttcgtggtgtagcggtgaaatgcttagatatcacgaagaactccgattgcgaaggcagcctgctaagctgcaactgacattgaggctcgaaagtgtgggtatcaaacaggattagataccctggtagtccacacggtaaacgatgaatactcgctgtttgcgatatacagcaagcggccaagcgaaagcgttaagtattccacctggggagtacgccggcaacggtgaaactcaaaggaattgacgggggcccgcacaagcggaggaacatgtggtttaattcgatgatacgcgaggaaccttacccgggcttaaattgcagatgaattacggtgaaagccgtaagccgcaaggcatctgtgaaggtgctgcatggttgtcgtcagctcgtgccgtgaggtgtcggcttaagtgccataacgagcgcaacccttgttgtcagttactaacaggttccgctgaggactctgacaagactgccatcgtaagatgtgaggaaggtggggatgacgtcaaatcagcacggcccttacgtccggggctacacacgtgttacaatggggggtacagagggccgctaccacgcgagtggatgccaatcccaaaaacctctctcagttcggactggagtctgcaacccgactccacgaagctggattcgctagtaatcgcgcatcagccacggcgcggtgaatacgttcccgggccttgtacacaccgcccgtcaagccatgggagccgggggtacctgaagtgcgtaaccgcgaggagcnc

SEQ ID NO: 66 - DNA, 1399 bp, synthetic construct
  misc_feature 1..1399: note = Synthetic: FBI00112
  source 1..1399: mol_type = other DNA
anncntgcagtcgaggggcagcatgaacttagcttgctaagtttgatggcgaccggcgcacgggtgagtaacacgtatccaacctgccgatgactcggggatagcctttcgaaagaaagattaatacccgatggcatagttcttccgcatggtagaactattaaagaatttcggtcatcgatggggatgcgttccattaggttgttggcggggtaacggcccaccaagccttcgatggataggggttctgagaggaaggtcccccacattggaactgagacacggtccaaactcctacgggaggcagcagtgaggaatattggtcaatggacgagagtctgaaccagccaagtagcgtgaaggatgactgccctatgggttgtaaacttcttttatacgggaataaagtgaggcacgtgtgcctttttgtatgtaccgtatgaataaggatcggctaactccgtgccagcagccgcggtaatacggaggatccgagcgttatccggatttattgggtttaaagggagcgtaggcggacgcttaagtcagttgtgaaagtttgcggctcaaccgtaaaattgcagttgatactgggtgtcttgagtacagtagaggcaggcggaattcgtggtgtagcggtgaaatgcttagatatcacgaagaactccgattgcgaaggcagcttgctggactgtaactgacgctgatgctcgaaagtgtgggtatcaaacaggattagataccctggtagtccacacagtaaacgatgaatactcgctgtttgcgatatacagtaagcggccaagcgaaagcgttaagtattccacctggggagtacgccggcaacggtgaaactcaaaggaattgacgggggcccgcacaagcggaggaacatgtggtttaattcgatgatacgcgaggaaccttacccgggcttgaattgcaactgaatgatgtggagacatgtcagccgcaaggcagttgtgaaggtgctgcatggttgtcgtcagctcgtgccgtgaggtgtcggcttaagtgccataacgagcgcaacccttatcgatagttaccatcaggttatgctggggactctgtcgagactgccgtcgtaagatgtgaggaaggtggggatgacgtcaaatcagcacggcccttacgtccggggctacacacgtgttacaatggggggtacagaaggcagctacacggtgacgtgatgctaatcccgaaagcctctctcagttcggattggagtctgcaacccgactccatgaagctggattcgctagtaatcgcgcatcagccacggcgcggtgaatacgttcccgggccttgtacacaccgcccgtcaagccatgaaagccgggggtacnnnnnagtgnntnnccgcaaggagcnc

SEQ ID NO: 67 - DNA, 1395 bp, synthetic construct
  misc_feature 1..1395: note = Synthetic: FBI00113
  source 1..1395: mol_type = other DNA
tgcagtcgaggggcagcatgatttgtagcaatacagattgatggcgaccggcgcacgggtgagtaacgcgtatgcaacttacctatcagagggggatagcccggcgaaagtcggattaataccccataaaacaggggtcccgcatgggaatatttgttaaagattcatcgctgatagataggcatgcgttccattaggcagttggcggggtaacggcccaccaaaccgacgatggataggggttctgagaggaaggtcccccacattggtactgagacacggaccaaactcctacgggaggcagcagtgaggaatattggtcaatggccgagaggctgaaccagccaagtcgcgtgaaggaagaaggatctatggtttgtaaacttcttttataggggaataaagtggaggacgtgtccttttttgtatgtaccctatgaataagcatcggctaactccgtgccagcagccgcggtaatacggaggatgcgagcgttatccggatttattgggtttaaagggtgcgtaggtggtgatttaagtcagcggtgaaagtttgtggctcaaccataaaattgccgttgaaactgggttacttgagtgtgtttgaggtaggcggaatgcgtggtgtagcggtgaaatgcatagatatcacgcagaactccgattgcgaaggcagcttactaaaccataactgacactgaagcacgaaagcgtggggatcaaacaggattagataccctggtagtccacgcagtaaacgatgattactaggagtttgcgatacaatgtaagctctacagcgaaagcgttaagtaatccacctggggagtacgccggcaacggtgaaactcaaaggaattgacgggggcccgcacaagcggaggaacatgtggtttaattcgatgatacgcgaggaaccttacccgggtttgaacgtagtctgaccggagtggaaacactctttctagcaatagcagattacgaggtgctgcatggttgtcgtcagctcgtgccgtgaggtgtcggcttaagtgccataacgagcgcaacccttatcactagttactaacaggtgaagctgaggactctggtgagactgccagcgtaagctgtgaggaaggtggggatgacgtcaaatcagcacggcccttacatccggggcgacacacgtgttacaatggcatggacaaagggcagctacctggtgacaggatgctaatctccaaaccatgtctcagttcggatcggagtctgcaactcgactccgtgaagctggattcgctagtaatcgcgcatcagccatggcgcggtgaatacgttcccgggccttgtacacaccgcccgtcaagccatgggagccgggggtacctgaagtccgtaaccgcaaggatc

SEQ ID NO: 68 - DNA, 1387 bp, synthetic construct
  misc_feature 1..1387: note = Synthetic: FBI00115
  source 1..1387: mol_type = other DNA
tgcagtcgagcgaagcacttaagttcgattcttcggatgaagacttttgtgactgagcggcggacgggtgagtaacgcgtgggtaacctgcctcatacagggggataacagttagaaatggctgctaataccgcataagaccacagtactgcatggtacagtggtaaaaactccggtggtatgagatggacccgcgtctgattaggtagttggtgaggtaacggcccaccaagccgacgatcagtagccgacctgagagggtgaccggccacattgggactgagacacggcccagactcctacgggaggcagcagtggggaatattgcacaatgggcgaaagcctgatgcagcgacgccgcgtgaaggatgaagtatttcggtatgtaaacttctatcagcagggaagaaaatgacggtacctgactaagaagccccggctaactacgtgccagcagccgcggtaatacgtagggggcaagcgttatccggatttactgggtgtaaagggagcgtagacggctgtgcaagtctgaagtgaaaggcatgggctcaacctgtggactgctttggaaactgtgcagctagagtgtcggagaggtaagtggaattcctagtgtagcggtgaaatgcgtagatattaggaggaacaccagtggcgaaggcggcttactggacgatgactgacgttgaggctcgaaagcgtggggagcaaacaggattagataccctggtagtccacgccgtaaacgatgactgctaggtgtcgggtagcaaagctattcggtgccgcagctaacgcaataagcagtccacctggggagtacgttcgcaagaatgaaactcaaaggaattgacggggacccgcacaagcggtggagcatgtggtttaattcgaagcaacgcgaagaaccttacctgatcttgacatcccgatgaccgcttcgtaatggaagtttttcttcggaacatcggtgacaggtggtgcatggttgtcgtcagctcgtgtcgtgagatgttgggttaagtcccgcaacgagcgcaacccttatcttcagtagccagcatttaagatgggcactctggagagactgccagggataacctggaggaaggtggggatgacgtcaaatcatcatgccccttatgaccagggctacacacgtgctacaatggcgtaaacaaagggaagcagagccgcgaggccgagcaaatctcaaaaataacgtctcagttcggattgtagtctgcaactcgactacatgaagctggaatcgctagtaatcgcagatcagaatgctgcggtgaatacgttcccgggtcttgtacacaccgcccgtcacaccatgggagtcagtaacgcccgaagtcagtgacccaacncgaaa

SEQ ID NO: 69 - DNA, 1526 bp, synthetic construct
  misc_feature 1..1526: note = Synthetic: FBI00116
  source 1..1526: mol_type = other DNA
tggagagtttgatcctggctcaggatgaacgctggcggcgtgcttaacacatgcaagtcgaacgaagcaccttgatttgattcttcggatgaaratcytggtgactgagtggcggacgggtgagtaacgcgtgggtaacctgcctcatacagggggataacagttagaaatgactgctaataccgcataagaccacagcaccgcatggtgcaggggtaaaaactccggtggtatgagatggacccgcgtctgattaggtagttggtggggtaacggcctaccaagccgacgatcagtagccgacctgagagggtgaccggccacattgggactgagacacggcccaaactcctacgggaggcagcagtggggaatattgcacaatgggggaaaccctgatgcagcgacgccgcgtgagcgatgaagtatttcggtatgtaaagctctatcagcagggaagaaaatgacggtacctgactaagaagcaccggctaaatacgtgccagcagccgcggtaatacgtatggtgcaagcgttatccggatttactgggtgtaaagggagcgtagacggagtggcaagtctgatgtgaaaacccggggctcaaccccgggactgcattggaaactgtcaatctrgagtaccggagaggtaagcggaattcctagtgtagcggtgaaatgcgtagatattaggaggaacaccagtggcgaaggcggcttactggacggtaactgacgttgaggctcgaaagcgtggggagcaaacaggattagataccctggtagtccacgccgtaaacgatgactactaggtgtcgggcagcaaagctgttcggtgccgcagcaaacgcaataagtagtccacctggggagtacgttcgcaagaatgaaactcaaaggaattgacggggacccgcacaagcggtggagcatgtggtttaattcgaagcaacgcgaagaaccttacctgctcttgacatctccctgaccggcaagtaatgttgcctttccttcgggacagggatgacaggtggtgcatggttgtcgtcagctcgtgtcgtgagatgttgggttaagtcccgcaacgagcgcaacccctatctttagtagccagcggyywggccgggcactctagagagactgccagggataacctggaggaaggtggggatgacgtcaaatcatcatgccccttatgagcagggctacacacgtgctacaatggcgtaaacaaagggaggcagaaccgcgaggtcgagcaaatcccaaaaataacgtctcagttcggattgtagtctgcaactcgactacatgaagctggaatcgctagtaatcgcgaatcagaatgtcgcggtgaatacgttcccgggtcttgtacacaccgcccgtcacaccatgggagtcagtaacgcccgaagtcagtgacccaaccgtaaggagggagctgccgaaggtgggaccgataactggggtgaagtcgtaacaaggtagccgtatcggaaggtgcggytggatcacctcctt

SEQ ID NO: 70 - DNA, 1526 bp, synthetic construct
  misc_feature 1..1526: note = Synthetic: FBI00117
  source 1..1526: mol_type = other DNA
tggagagtttgatcctggctcaggatgaacgctggcggcgtgcttaacacatgcaagtcgaacgggaaayayttyattgarrcttcggyvgatttrryytrtttctagtggcggacgggtgagtaacgcgtgggtaacctgccttatactgggggataacagccagaaatgrctgctaataccgcataagcgcacagaaccgcatggttcsgtgtgaaaaactccggtggtataagatggacccgcgttggattagctagttggcagggcarcggcctaccaaggcgacgatccatagccggcctgagagggtgaacggccacattgggactgagacacggcccagactcctacgggaggcagcagtggggaatattgcacaatgggggaaaccctgatgcagcgacgccgcgtgaaggaagaagtatctcggtatgtaaacttctatcagcagggaagataatgacggtacctgactaagaagccccggctaactacgtgccagcagccgcggtaatacgtagggggcaagcgttatccggatttactgggtgtaaagggagcgtagacggcgcagcaagtctgatgtgaaaggcaggggcttaacccctggactgcattggaaactgctgtgcttgagtgccggaggggtaagcggaattcctagtgtagcggtgaaatgcgtagatattaggaggaacaccagtggcgaaggcggcttactggacggtaactgacgttgaggctcgaaagcgtggggagcaaacaggattagataccctggtagtccacgccgtaaacgatgaatactaggtgtcwrggagcacagctcttwggtgccgccgcaaacgcattaagtattccacctggggagtacgttcgcaagaatgaaactcaaaggaattgacggggacccgcacaagcggtggagcatgtggtttaattcgaagcaacgcgaagaaccttaccaaatcttgacatccctctgaccgggacttaaccgtccctttycttcgggacaggggagacaggtggtgcatggttgtcgtcagctcgtgtcgtgagatgttgggttaagtcccgcaacgagcgcaacccctatccttagtagccagcrsgtmrkgsygggcactctgaggagactgccagggataacctggaggaaggcggggatgacgtcaaatcatcatgccccttatgatttgggctacacacgtgctacaatggcgtaaacaaagggaagcgaaccygygagggtgggcaaatctcaaaaataacgtcccagttcggactgcagtctgcaactcgactgcacraagctggaatcgctagtaatcgcggatcagaatgccgcggtgaatacgttcccgggtcttgtacacaccgcccgtcacaccatgggagtcagtaacgcccgaagtcagtgacctaacygcaargraggagctgccgaaggcgggaccgatgactggggtgaagtcgtaacaaggtagccgtaccggaaggtgcggytggatcacctcctt

SEQ ID NO: 71 - DNA, 1397 bp, synthetic construct
  misc_feature 1..1397: note = Synthetic: FBI00120
  source 1..1397: mol_type = other DNA
tgnnagtcgagcgaagcggntcttcggaagttttcggatggaanaggaattgacttagcggcggacgggtgagtaacgcgtgggtaacctgccttacactgggggataacagttagaaatgactgctaataccgcataagcgcacagggccgcatggcctggtgtgaaaaactccggtggtgtaagatggacccgcgtctgattaggtagttggtggggtaacggcccaccaagccgacgatcagtagccgacctgagagggtgaccggccacattgggactgagacacggcccaaactcctacgggaggcagcagtggggaatattggacaatgggcgaaagcctgatccagcgacgccgcgtgagtgaagaagtatttcggtatgtaaagctctatcagcagggaagaaaaatgacggtacctgactaagaagccccggctaactacgtgccagcagccgcggtaatacgtagggggcaagcgttatccggatttactgggtgtaaagggagcgtagacggtttagcaagtctgaagtgaaagcccggggctcaaccccggtactgctttggaaactgttagacttgagtgcaggagaggtaagtggaattcctagtgtagcggtgaaatgcgtagatattaggaggaacaccagtggcgaaggcggcttactggactgtaactgacgttgaggctcgaaagcgtggggagcaaacaggattagataccctggtagtccacgccgtaaacgatgaatactaggtgtcggggggcaaagcccttcggtgccgccgcaaacgcaataagtattccacctggggagtacgttcgcaagaatgaaactcaaaggaattgacggggacccgcacaagcggtggagcatgtggtttaattcgaagcaacgcgaagaaccttaccaagtcttgacatcccactgaaaacactttaaccggtgtccctcttcggagcagtggagacaggtggtgcatggttgtcgtcagctcgtgtcgtgagatgttgggttaagtcccgcaacgagcgcaacccttatccttagtagccagcgagtagagtcgggcactctggggagactgccagggataacctggaggaaggtggggatgacgtcaaatcatcatgccccttatgatttgggctacacacgtgctacaatggcgtaaacaaagggaggcaaaggagcgatctggagcaaaccccaaaaataacgtctcagttcggattgcaggctgcaactcgcctgcatgaagctggaatcgctagtaatcgcgaatcagaatgtcgcggtgaatacgttcccgggtcttgtacacaccgcccgtcacaccatgggagttggtaacgcccgaagtcagtgacccaaccgnaaggagggag

SEQ ID NO: 72 - DNA, 1396 bp, synthetic construct
  misc_feature 1..1396: note = Synthetic: FBI00123
  source 1..1396: mol_type = other DNA
tgcaagtcgaacgaagcactttaattgatttcttcggaatgaagtttttgtgactgagtggcggacgggtgagtaacgcgtgggtaacctgcctcatacagggggataacagttggaaacgactgctaataccgcataagcgcacaggattgcatgatccagtgtgaaaaactccggtggtatgagatggacccgcgtctgattagccagttggcggggtaacggcccaccaaagcgacgatcagtagccgacctgagagggtgaccggccacattgggactgagacacggcccaaactcctacgggaggcagcagtggggaatattgcacaatgggggaaaccctgatgcagcgacgccgcgtgagcgaagaagtatttcggtatgtaaagctctatcagcagggaagaagaatgacggtacctgactaagaagcaccggctaaatacgtgccagcagccgcggtaatacgtatggtgcaagcgttatccggatttactgggtgtaaagggagcgcaggcggtacggcaagtctgatgtgaaatcccggggctcaaccccggtactgcattggaaactgtcggactagagtgtcggaggggtaagtggaattcctagtgtagcggtgaaatgcgtagatattaggaggaacaccagtggcgaaggcggcttactggacgattactgacgctgaggctcgaaagcgtggggagcaaacaggattagataccctggtagtccacgccgtaaacgatgaatactaggtgtcggggagcattgctcttcggtgccgcagcaaacgcaataagtattccacctggggagtacgttcgcaagaatgaaactcaaaggaattgacggggacccgcacaagcggtggagcatgtggtttaattcgaagcaacgcgaagaaccttaccaagtcttgacatcccactgacaaagtatgtaatgtactttctcttcggagcagtggtgacaggtggtgcatggttgtcgtcagctcgtgtcgtgagatgttgggttaagtcccgcaacgagcgcaacccctattcttagtagccagcggttcggccgggcactctagggagactgccagggataacctggaggaaggtggggatgacgtcaaatcatcatgccccttatgacttgggctacacacgtgctacaatggcgtaaacaaagggaagcaatcccgcgagggggagcaaatctcaaaaataacgtctcagttcggactgtagtctgcaactcgactacacgaagctggaatcgctagtaatcgcgaatcagaatgtcgcggtgaatacgttcccgggtcttgtacacaccgcccgtcacaccatgggagttggtaatgcccgaagtcagtgacccaaccgcaaggagggag

SEQ ID NO: 73 - DNA, 1382 bp, synthetic construct
  misc_feature 1..1382: note = Synthetic: FBI00124
  source 1..1382: mol_type = other DNA
tcgaacgaaacaccttatttgattttcttcggaactgaagatttggtgattgagtggcggacgggtgagtaacgcgtgggtaacctgccctgtacagggggataacagtcagaaatgactgctaataccgcataagaccacagcaccgcatggtgcaggggtaaaaactccggtggtacaggatggacccgcgtctgattagctggttggtgaggtaacggctcaccaaggcgacgatcagtagccggcttgagagagtgaacggccacattgggactgagacacggcccaaactcctacgggaggcagcagtggggaatattgcacaatgggggaaaccctgatgcagcgacgccgcgtgagtgaagaagtatcttcggtatgtaaagctctatcagcagggaagaaaatgacggtacctgactaagaagccccggctaactacgtgccagcagccgcggtaatacgtagggggcaagcgttatccggaattactgggtgtaaagggtgcgtaggtggtatggcaagtcagaagtgaaaacccagggcttaactctgggactgcttttgaaactgtcagactggagtgcaggagaggtaagcggaattcctagtgtagcggtgaaatgcgtagatattaggaggaacatcagtggcgaaggcggcttactggactgaaactgacactgaggcacgaaagcgtggggagcaaacaggattagataccctggtagtccacgccgtaaacgatgaatactaggtgtcggggccgtagaggcttcggtgccgcagccaacgcagtaagtattccacctggggagtacgttcgcaagaatgaaactcaaaggaattgacggggacccgcacaagcggtggagcatgtggtttaattcgaagcaacgcgaagaaccttacctggtcttgacatccttctgaccggtccttaaccggacctttccttcgggacaggagagacaggtggtgcatggttgtcgtcagctcgtgtcgtgagatgttgggttaagtcccgcaacgagcgcaacccctatctttagtagccagcatataaggtgggcactctagagagactgccagggataacctggaggaaggtggggacgacgtcaaatcatcatgccccttatgaccagggctacacacgtgctacaatggcgtaaacagagggaagcagcctcgtgagagtgagcaaatcccaaaaataacgtctcagttcggattgtagtctgcaactcgactacatgaagctggaatcgctagtaatcgcgaatcagaatgtcgcggtgaatacgttcccgggtcttgtacacaccgcccgtcacaccatgggagtcagtaacgcccgaagtcagtgacccaaccgt

SEQ ID NO: 74 - DNA, 1379 bp, synthetic construct
  misc_feature 1..1379: note = Synthetic: FBI00125
  source 1..1379: mol_type = other DNA
gcaagtcgaggggcagcatcatcaaagcttgctttgatggatggcgaccggcgcacgggtgagtaacacgtatccaacctgccgacaacactgggatagcctttcgaaagaaagattaataccggatggcatagttttcccgcatgggataattattaaagaatttcggttgtcgatggggatgcgttccattaggcagttggcggggtaacggcccaccaaaccaacgatggataggggttctgagaggaaggtcccccacattggaactgagacacggtccaaactcctacgggaggcagcagtgaggaatattggtcaatggacgagagtctgaaccagccaagtagcgtgaaggatgactgccctatgggttgtaaacttcttttatacgggaataaagtgagccacgtgtggctttttgtatgtaccgtatgaataaggatcggctaactccgtgccagcagccgcggtaatacggaggatccgagcgttatccggatttattgggtttaaagggagcgtaggcgggttgttaagtcagttgtgaaagtttgcggctcaaccgtaaaattgcagttgatactggcgaccttgagtgcaacagaggtaggcggaattcgtggtgtagcggtgaaatgcttagatatcacgaagaactccgattgcgaaggcagcttactggattgtaactgacgctgatgctcgaaagtgtgggtatcaaacaggattagataccctggtagtccacacagtaaacgatgaatactcgctgttggcgatatacagtcagcggccaagcgaaagcattaagtattccacctggggagtacgccggcaacggtgaaactcaaaggaattgacgggggcccgcacaagcggaggaacatgtggtttaattcgatgatacgcgaggaaccttacccgggcttaaattgcaactgacggaaccggaaacggttctttcttcggacagttgtgaaggtgctgcatggttgtcgtcagctcgtgccgtgaggtgtcggcttaagtgccataacgagcgcaacccttacgggtagttaccatcaggttatgctggggactctacccggactgccgtcgtaagatgtgaggaaggtggggatgacgtcaaatcagcacggcccttacgtccggggctacacacgtgttacaatggggggtacagaaggcagctacacggcgacgtggtgctaatcccgaaagcctctctcagttcggattggagtctgcaacccgactccatgaagctggattcgctagtaatcgcgcatcagccacggcgcggtgaatacgttcccgggccttgtacacaccgcccgtcaagccatgaaagccgggggtacctnaagtacgta

SEQ ID NO: 75 - DNA, 1372 bp, synthetic construct
  misc_feature 1..1372: note = Synthetic: FBI00126
  source 1..1372: mol_type = other DNA
tgcagtcgaacgggancccaggagcttgctcctgggtgagagtggcgaacgggtgagtaatgcgtgaccgacctgccccatacaccggaatagctcctggaaacgggtggtaatgccggatgctccagttggatgcatgtccttctgggaaagattcatcggtatgggatggggtcgcgtcctatcagcttgatggcggggtaacggcccaccatggcttcgacgggtagccggcctgagagggcgaccggccacattgggactgagatacggcccagactcctacgggaggcagcagtggggaatattgcacaatgggcgcaagcctgatgcagcgacgccgcgtgcgggatgacggccttcgggttgtaaaccgcttttgactgggagcaagcccttcggggtgagtgtacctttcgaataagcaccggctaactacgtgccagcagccgcggtaatacgtagggtgcaagcgttatccggaattattgggcgtaaagggctcgtaggcggttcgtcgcgtccggtgtgaaagtccatcgcttaacggtggatccgcgccgggtacgggcgggcttgagtgcggtaggggagactggaattcccggtgtaacggtggaatgtgtagatatcgggaagaacaccaatggcgaaggcaggtctctgggccgtcactgacgctgaggagcgaaagcgtggggagcgaacaggattagataccctggtagtccacgccgtaaacggtggatgctggatgtggggaccattccacggtctccgtgtcggagccaacgcgttaagcatcccgcctggggagtacggccgcaaggctaaaactcaaagaaattgacgggggcccgcacaagcggcggagcatgcggattaattcgatgcaacgcgaagaaccttacctgggcttgacatgttcccgacagccccagagatggggcctcccttcggggcgggttcacaggtggtgcatggtcgtcgtcagctcgtgtcgtgagatgttgggttaagtcccgcaacgagcgcaaccctcgccctgtgttgccagcacgtcgtggtgggaactcacgggggaccgccggggtcaactcggaggaaggtggggatgacgtcagatcatcatgccccttacgtccagggcttcacgcatgctacaatggccggtacaacgggatgcgacactgtgaggtggagcggatcccttaaaaccggtttcagttcggattggagtttgcaacccgactccatgaaggcggagtcgctagtaatcgcggatcagcaacgccgcggtgaatgcgttcccgggccttgtacacaccgcccgtcaagtcatgaaagtgggtagcacccgaagccggtggcc

SEQ ID NO: 76 - DNA, 1190 bp, synthetic construct
  misc_feature 1..1190: note = Synthetic: FBI00127
  source 1..1190: mol_type = other DNA
nnnnnnnnnntgnaagtcgaacggcacctgccctcgggcagaagcgagtggcgaacggctgagtaacacgtggagaacctgccccctcccccgggatagccgcccgaaaggacgggtaataccggataccccggggtgccgcatggcaccccggctaaagccccgacgggaggggatggctccgcggcccatcaggtagacggcggggtgacggcccaccgtgccgacaacgggtagccgggttgagagaccgaccggccagattgggactgagacacggcccagactcctacgggaggcagcagtggggaatcttgcgcaatggggggaaccctgacgcagcgacgccgcgtgcgggacggaggccttcgggtcgtaaaccgctttcagcagggaagagtcaagactgtacctgcagaagaagccccggctaactacgtgccagcagccgcggtaatacgtagggggcgagcgttatccggattcattgggcgtaaagcgcgcgtaggcggcccggcaggccgggggtcgaagcggggggctcaaccccccgaagcccccggaacctccgcggcttgggtccggtaggggagggtggaacacccggtgtagcggtggaatgcgcagatatcgggtggaacaccggtggcgaaggcggccctctgggccgagaccgacgctgaggcgcgaaagctgggggagcgaacaggattagataccctggtagtcccagccgtaaacgatggacgctaggtgtgggggggggnnnatncccccctgncncncncnacncacttnnngncncncccgnggagnnnngncncnnngngtaaantctnannnnttnacngggggncnnncnaannnnngnagnatgtggnttnnntttnnannnnncgnannnnntttnnnnggtnnacatatgggngnnacgggggnnancccnngggnnnnnangnnnccctanngnnggngcanngctnnnncnannnnnnnnnngnnnnnnnnnnggnnnnnnccnnnnnnnnnnnnnnnccnnccncgnngnntnnnnnnnnnggngnnngnncnngnancnnnnnnnnncnnnnccnnnnnnnggnnnanannnnngnnnnnnnnnnntnnnnnnnnnnnnnnnnnnnnnnnnnnnnnnnannnnnnnnnnnnnncnncnnngggannnnnan

SEQ ID NO: 77 - DNA, 1402 bp, synthetic construct
  misc_feature 1..1402: note = Synthetic: FBI00128
  source 1..1402: mol_type = other DNA
annnntgcangtcgagcgaagcggtttcgatgaagttttcggatggatttgaaatcgacttagcggcggacgggtgagtaacgcgtgggtaacctgccttacactgggggataacagttagaaatgactgctaataccgcataagcgcacagggccgcatggtctggtgtgaaaaactccggtggtgtaagatggacccgcgtctgattaggtagttggtggggtaacggcccaccaagccgacgatcagtagccgacctgagagggtgaccggccacattgggactgagacacggcccaaactcctacgggaggcagcagtggggaatattggacaatgggcgaaagcctgatccagcgacgccgcgtgagtgaagaagtatttcggtatgtaaagctctatcagcagggaagaaaatgacggtacctgactaagaagccccggctaactacgtgccagcagccgcggtaatacgtagggggcaagcgttatccggatttactgggtgtaaagggagcgtagacggttaagcaagtctgaagtgaaagcccggggctcaaccccggtactgctttggaaactgtttgacttgagtgcaggagaggtaagtggaattcctagtgtagcggtgaaatgcgtagatattaggaggaacaccagtggcgaaggcggcttactggactgtaactgacgttgaggctcgaaagcgtggggagcaaacaggattagataccctggtagtccacgccgtaaacgatgaatactaggtgtcgggggacaaagtccttcggtgccgccgctaacgcaataagtattccacctggggagtacgttcgcaagaatgaaactcaaaggaattgacggggacccgcacaagcggtggagcatgtggtttaattcgaagcaacgcgaagaaccttaccaagtcttgacatcccattgaaaatcatttaaccgtgatccctcttcggagcaatggagacaggtggtgcatggttgtcgtcagctcgtgtcgtgagatgttgggttaagtcccgcaacgagcgcaacccttatccttagtagccagcaagtaatgttgggcactctggggagactgccagggataacctggaggaaggtggggatgacgtcaaatcatcatgccccttatgatttgggctacacacgtgctacaatggcgtaaacaaagggaagcaaaggagcgatctggagcaaaccccaaaaataacgtctcagttcggattgcaggctgcaactcgcctgcatgaagctggaatcgctagtaatcgcgaatcagaatgtcgcggtgaatacgttcccgggtcttgtacacaccgcccgtcacaccatgggagttggtaacgcccgaagtcagtgacccaaccgtaaggagggagc

SEQ ID NO: 78 - DNA, 1341 bp, synthetic construct
  misc_feature 1..1341: note = Synthetic: FBI00132
  source 1..1341: mol_type = other DNA
tcgaacgattaagacggctccggccgtgaatagagtggcgaacgggtgagtaacacgtgaccaacctgccccctaccccgggataacgcaaggaaacctgcgctaataccggatactccttcccctgctcctgcaggggtcgggaaagcccagacggtaggggatggggtcgcggcccattaggtagacggcggggcaacggcccaccgtgcctgcgatgggtagccgggttgagagaccgaccggccacattgggactgagatacggcccagactcctacgggaggcagcagtggggaattttgcgcaatggggggaaccctgacgcagcaacgccgcgtgcgggatgaaggccttcgggttgtaaaccgctttcagcagggaagatagtgacggtacctgcagaagaagccccggctaactacgtgccagcagctnnggtaatacgtagggggcgagcgttatccggattcattgggcgtaaagcgcgcgtaggcggcccgtcaagcggaacctctaacccgagggctcaacccccggtcgggttccgaactggcaggctcgagtttggtagaggaagatggaattcccggtgtagcggtggaatgcgcagatatcgggaagaacaccgatggcgaaggcagtcttctgggccatcaactgacgctgangcgcgaaagctgggggagcgaacaggattagataccctggtagtcccagccgtaaacgatgggtgctaggtgtggggggatcatccctccgtgccgcagccaacgcattaagcaccccgcctggggagtacggccgcaaggctaaaactcaaaggaattgacgggggcccgcacaagcagcggagcatgtggcttaattcgaagccaacgcgaagaaccttaccagggcttgacatgctagtgaagccgggaaacccggtggccgagaggagctagcgcaggtggtgcatggctgtcgtcagctcgtgtcgtgagatgttgggttaagtcccgcaacgagcgcaacccctatcgtatgttgccagcattgagttggggactcatacgagacngccggcgtcaagccggaggaaggtggggacgacgtcaagtcatcatgccctttatgccctgggctgcacacgtgctacaatggccggtacaacgggccgcgacctggcgacaggaagcgaatccctcaaagccggccccagttcggatcggaggctgcaacccgcctccgtgaagtcggagttgctagtaatcgcggatcagcatgccgcggtgaatacgttcccgggccttgtacacaccgcccgtcacaccacccgagtcgtctgcacccgaagcc

SEQ ID NO: 79 - DNA, 1395 bp, synthetic construct
  misc_feature 1..1395: note = Synthetic: FBI00133
  source 1..1395: mol_type = other DNA
agtcgaacggcagcatgggggcaaccctgatggcgagtggcgaacgggtgagtaatatatcggaacgtacccaagagtgggggacaaccagtcgaaagattggctaataccgcatacgatctaaggatgaaagtgggggaccttcgggcctcacgctcctggagcggccgatagctgattagctagttggtggggtaaaggcctaccaaggcgatgatcagtagctggtctgagaggacgatcagccacactgggactgagacacggcccagactcctacgggaggcagcagtggggaattttggacaatgggggaaaccctgatccagcaatgccgcgtgagtgaagaaggccttcgggttgtaaagctcttttgtcagggaagaaattgattgggctaatatcccgattagatgacggtacctgaagaataagcaccggctaactacgtgccagcagccgcggtaatacgtagggtgcgagcgttaatcggaattactgggcgtaaagcgtgcgcaggcggttgtgtaagacagatgtgaaatgcccgggctcaacctgggaattgcatttgtgactgcacggctagagtgtgtcagaggggggtagaattccacgtgtagcagtgaaatgcgtagatatgtggaggaataccgatggcgaaggcagccccctgggataacactgacgctcatgcacgaaagcgtggggagcaaacaggattagataccctggtagtccacgccctaaacgatgtcgactagttgttgggtcttaattgacttagtaacgcagctaacgcgtgaagtcgaccgcctggggagtacggtcgcaagattaaaactcaaaggaattgacggggacccgcacaagcggtggatgatgtggattaattcgatgcaacgcgaaaaaccttacctacccttgacatgtcaggaatcccgaagagatttgggagtgctcgaaagagagcctgaacacaggtgctgcatggctgtcgtcagctcgtgtcgtgagatgttgggttaagtcccgcaacgagcgcaacccttgtcattagttgctacgaaagggcactctaatgaaactgccggtgacaaaccggaggaaggtggggatgacgtcaagtcctcatggcccttatgggtagggcttcacacgtcatacaatggtgcatacagagggttgccaacccgagagggggagctaatctcagaaagtgcatcgtagtccggattgaagtctgcaactcgacttcatgaagttggaatcgctagtaatcgcggatcagcatgtcgcggtgaatacgttcccgggtcttgtacacaccgcccgtcacaccatgggagcgggttttaccagaagtaggtagcttaaccgtaaggagggcnntnc

SEQ ID NO: 80 - DNA, 1389 bp, synthetic construct
  misc_feature 1..1389: note = Synthetic: FBI00135
  source 1..1389: mol_type = other DNA
agtcgaacgggnnccatcaggctttgcttggtggtgagagtggcgaacgggtgagtaatgcgtgaccgacctgccccatacaccggaatagctcctggaaacgggtggtaatgccggatgctccgactcctcgcatggggtgtcgggaaagattatatcggtatgggatggggtcgcgtcctatcaggtagtcggcggggtaacggcccaccgagcctacgacgggtagccggcctgagagggcgaccggccacattgggactgagatacggcccagactcctacgggaggcagcagtggggaatattgcacaatgggcgcaagcctgatgcagcgacgccgcgtgcgggatgacggccttcgggttgtaaaccgcttttgatcgggagcaagccttcgggtgagtgtacctttcgaataagcaccggctaactacgtgccagcagccgcggtaatacgtagggtgcaagcgttatccggaattattgggcgtaaagggctcgtaggcggttcgtcgcgtccggtgtgaaagtccatcgcttaacggtggatctgcgccgggtacgggcgggctggagtgcggtaggggagactggaattcccggtgtaacggtggaatgtgtagatatcgggaagaacaccaatggcgaaggcaggtctctgggccgttactgacgctgaggagcgaaagcgtggggagcgaacaggattagataccctggtagtccacgccgtaaacggtggatgctggatgtggggcccgttccacgggttccgtgtcggagctaacgcgttaagcatcccgcctggggagtacggccgcaaggctaaaactcaaagaaattgacgggggcccgcacaagcggcggagcatgcggattaattcgatgcaacgcgaagaaccttacctgggcttgacatgttcccgacagccgtagagatatggcctcccttcggggcgggttcacaggtggtgcatggtcgtcgtcagctcgtgtcgtgagatgttgggttaagtcccgcaacgagcgcaaccctcgccctgtgttgccagcacgtcatggtgggaactcacgggggaccgccggggtcaactcggaggaaggtggggatgacgtcagatcatcatgccccttacgtccagggcttcacgcatgctacaatggccggtacaacgggatgcgacacggcgacgtggagcggatccctgaaaaccggtctcagttcggattggagtctgcaacccgactccatgaaggcggagtcgctagtaatcgcggatcagcaacgccgcggtgaatgcgttcccgggccttgtacacaccgcccgtcaagtcatgaaagtgggtagcacccgaagccggtggcctaaccctttgngganggagc

SEQ ID NO: 81 - DNA, 1384 bp, synthetic construct
  misc_feature 1..1384: note = Synthetic: FBI00137
  source 1..1384: mol_type = other DNA
tcgaggggcatcaggaagaaagcttgctttctttgctggcgaccggcgcacgggtgagtaacacgtatccaacctgccctttactcggggatagcctttcgaaagaaagattaatacccgatagcataatgattccgcatggtttcattattaaaggattccggtaaaggatggggatgcgttccattaggttgttggtgaggtaacggctcaccaagccttcgatggataggggttctgagaggaaggtcccccacattggaactgagacacggtccaaactcctacgggaggcagcagtgaggaatattggtcaatgggcgctagcctgaaccagccaagtagcgtgaaggatgaaggctctatgggtcgtaaacttcttttatataagaataaagtgcagtatgtatactgttttgtatgtattatatgaataaggatcggctaactccgtgccagcagccgcggtaatacggaggatccgagcgttatccggatttattgggtttaaagggagcgtaggtggactggtaagtcagttgtgaaagtttgcggctcaaccgtaaaattgcagttgatactgtcagtcttgagtacagtagaggtgggcggaattcgtggtgtagcggtgaaatgcttagatatcacgaagaactccgattgcgaaggcagctcactggactgcaactgacactgatgctcgaaagtgtgggtatcaaacaggattagataccctggtagtccacacagtaaacgatgaatactcgctgtttgcgatatacagtaagcggccaagcgaaagcattaagtattccacctggggagtacgccggcaacggtgaaactcaaaggaattgacgggggcccgcacaagcggaggaacatgtggtttaattcgatgatacgcgaggaaccttacccgggcttaaattgcagtggaatgatgtggaaacatgtcagtgagcaatcaccgctgtgaaggtgctgcatggttgtcgtcagctcgtgccgtgaggtgtcggcttaagtgccataacgagcgcaacccttatcttcagttactaacaggtcatgctgaggactctggagagactgccgtcgtaagatgtgaggaaggtggggatgacgtcaaatcagcacggcccttacgtccggggctacacacgtgttacaatggggggtacagaaggcagctagcgggtgaccgtatgctaatcccaaaatcctctctcagttcggatcgaagtctgcaacccgacttcgtgaagctggattcgctagtaatcgcgcatcagccacggcgcggtgaatacgttcccgggccttgtacacaccgcccgtcaagccatgggagccgggggtacctgaagtacgnaaccgcaag

SEQ ID NO: 82 - DNA, 1414 bp, synthetic construct
  misc_feature 1..1414: note = Synthetic: FBI00140
  source 1..1414: mol_type = other DNA
gcagtcgaacggagaattttatttcggtagaattcttagtggcgaacgggtgagtaacgcgtaggcaacctgccctttagacggggacaacattccgaaaggagtgctaataccggatgtgatcatcttgccgcatggcaggatgaagaaagatggcctctacaagtaagctatcgctaaaggatgggcctgcgtctgattagctagttggtagtgtaacggactaccaaggcgatgatcagtagccggtctgagaggatgaacggccacattgggactgagacacggcccaaactcctacgggaggcagcagtggggaatcttccgcaatggacgaaagtctgacggagcaacgccgcgtgagtgatgaaggatttcggtctgtaaagctctgttgtttatgacgaacgtgcagtgtgtgaacaatgcattgcaatgacggtagtaaacgaggaagccacggctaactacgtgccagcagccgcggtaatacgtaggtggcgagcgttgtccggaattattgggcgtaaagagcatgtaggcggcttaataagtcgagcgtgaaaatgcggggctcaaccccgtatggcgctggaaactgttaggcttgagtgcaggagaggaaaggggaattcccagtgtagcggtgaaatgcgtagatattgggaggaacaccagtggcgaaggcgcctttctggactgtgtctgacgctgagatgcgaaagccagggtagcgaacgggattagataccccggtagtcctggccgtaaacgatgggtactaggtgtaggaggtatcgaccccttctgtgccggagttaacgcaataagtaccccgcctggggagtacggccgcaaggttgaaactcaaaggaattgacgggggcccgcacaagcggtggagtatgtggtttaattcgacgcaacgcgaagaaccttaccaaggcttgacattgattgaacgctctagagatagagctttcccttcggggacaagaaaacaggtggtgcatggctgtcgtcagctcgtgtcgtgagatgttgggttaagtcccgcaacgagcgcaacccctatcctatgttaccagcaagtaaagttggggactcatgggagactgccagggacaacctggaggaaggcggggatgacgtcaagtcatcatgccccttatgtcttgggctacacacgtactacaatggtcggaaacagagggaagcgaagccgcgaggcagagcaaaccccagaaacccgatctcagttcggatcgcaggctgcaacccgcctgcgtgaagtcggaatcgctagtaatcgcaggtcagcatactgcggtgaatacgttcccgggccttgtacacaccgcccgtcacaccacgaaagttggtaacacccgaagccggtgaggtaanctattag

SEQ ID NO: 83 - DNA, 1230 bp, synthetic construct
  misc_feature 1..1230: note = Synthetic: FBI00145
  source 1..1230: mol_type = other DNA
nnnnnnnnnagtcgaacgggancccaggagcttgctcctgggtgagagtggcgaacgggtgagtaatgcgtgaccgacctgccccatacaccggaatagctcctggaaacgggtggtaatgccggatgctccagttggatgcatgtccttctgggaaagattcatcggtatgggatggggtcgcgtcctatcagcttgatggcggggtaacggcccaccatggcttcgacgggtagccggcctgagagggcgaccggccacattgggactgagatacggcccagactcctacgggaggcagcagtggggaatattgcacaatgggcgcaagcctgatgcagcgacgccgcgtgcgggatgacggccttcgggttgtaaaccgcttttgactgggagcaagcccttcggggtgagtgtacctttcgaataagcaccggctaactacgtgccagcagccgcggtaatacgtagggtgcaagcgttatccggaattattgggcgtaaagggctcgtaggcggttcgtcgcgtccggtgtgaaagtccatcgcttaacggtggatccgcgccgggtacgggcgggcttgagtgcggtaggggagactggaattcccggtgtaacggtggaatgtgtagatatcgggaagaacaccaatggcgaaggcaggtctctgggccgtcactgacgctgaggagcgaaagcgtggggagcgaacaggattagataccctggtagtccacgccgtaaacggtggatgctggatgtggggaccattccacggtctccgtgtcggagccaacgcgttaagcatcccgcctggggagtacggccgcaaggctaaaactcaaagaaattgacgggggcccgcacaagcggcggancatgcggattaattcgatgcaacgcgaagaaccttacctgggcttgacatgttcccgacagcnntagagatnnngtctcccttcggggcgggntcacagnggngcatggncgtcgtcagctcgtgtcgtgagangntgggttaagtcccgcaacgancgcaaccctcgccctnnnnnncnncacgtcnngnnggnnntcacgggggaccnnngggnnactnnnnaaggtggggntnacgtcnnatcatcatncnnnnncgtcaggnntnnncgcanncnacnannnnnnnnnnnnngnnnnngnnannnnnnnnnnccnnaacnngnnnncnnntnnnnnnntnnnnnan

SEQ ID NO: 84 - DNA, 1392 bp, synthetic construct
  misc_feature 1..1392: note = Synthetic: FBI00147
  source 1..1392: mol_type = other DNA
tgcagtcgnncgaagcaattaaaatgaagttttcggatggatttttgattgactgagtggcggacgggtgagtaacgcgtggataacctgcctcacactgggggataacagttagaaatgactgctaataccgcataagcgcacagtaccgcagggtacggtgtgaaaaactccggtggtgtgagatggatccgcgtctgattagccagttggcggggtaacggcccaccaaagcgacgatcagtagccgacctgagagggtgaccggccacattgggactgagacacggcccaaactcctacgggaggcagcagtggggaatattgcacaatgggcgaaagcctgatgcagcgacgccgcgtgagtgaagaagtatttcggtatgtaaagctctatcagcagggaagaaaatgacggtacctgactaagaagccccggctaactacgtgccagcagccgcggtaatacgtagggggcaagcgttatccggatttactgggtgtaaagggagcgtagacggcgaagcaagtctgaagtgaaaacccagggctcaaccctgggactgctttggaaactgttttgctagagtgtcggagaggtaagtggaattcctagtgtagcggtgaaatgcgtagatattaggaggaacaccagtggcgaaggcggcttactggacgataactgacgttgaggctcgaaagcgtggggagcaaacaggattagataccctggtagtccacgccgtaaacgatgaatgctaggtgttggggggcaaagcccttcggtgccgtcgcaaacgcagtaagcattccacctggggagtacgttcgcaagaatgaaactcaaaggaattgacggggacccgcacaagcggtggagcatgtggtttaattcgaagcaacgcgaagaaccttaccaagtcttgacatcctcttgaccggcgtgtaacggcgccttcccttcggggcaagagagacaggtggtgcatggttgtcgtcagctcgtgtcgtgagatgttgggttaagtcccgcaacgagcgcaacccttatccttagtagccagcaggtaaagctgggcactctagggagactgccagggataacctggaggaaggtggggatgacgtcaaatcatcatgccccttatgatttgggctacacacgtgctacaatggcgtaaacaaagggaagcaagacagtgatgtggagcaaatcccaaaaataacgtcccagttcggactgtagtctgcaacccgactacacgaagctggaatcgctagtaatcgcgaatcagaatgtcgcggtgaatacgttcccgggtcttgtacacaccgcccgtcacaccatgggagtcagcaacgcccgaagtcagtgacccaactcgcaagaga

SEQ ID NO: 85 - DNA, 1393 bp, synthetic construct
  misc_feature 1..1393: note = Synthetic: FBI00149
  source 1..1393: mol_type = other DNA
tgcagtcgagcgagaaatttttaacggattccttcggggagaagataaggatggaaagcggcggacgggtgagtaacgcgtgagtaacctgcctttaggagggggacaacattccgaaagggatgctaataccgcataaaattattgtatcgcatggtatgataatcaaagatttatcgcctaaagatggactcgcgtccgattagctagttggtggggtaaaagcctaccaaggcgacgatcggtagccgaactgagaggttgatcggccacattgggactgagacacggcccagactcctacgggaggcagcagtgggggatattgcgcaatgggggaaaccctgacgcagcaacgccgcgtgaaggaagaaggccttcgggttgtaaacttctttaagtgtggaagataatgacggtacacacagaataagccacggctaactacgtgccagcagccgcggtaatacgtaggtggcaagcgttgtccggatttactgggtgtaaagggcgtgtaggcgggtagacaagtcagatgtgaaataccggggctcaactccggggctgcatttgaaactgtatatcttgagtgtcggagaggaaagcggaattcctagtgtagcggtgaaatgcgtagatattaggaggaacaccagtggcgaaggcggctttctggacgataactgacgctgaggcgcgaaagcgtggggagcaaacaggattagataccctggtagtccacgccgtaaacgatggatactaggtgtaggaggtatcgaccccttctgtgccgcagttaacacaataagtatcccacctggggagtacggtcgcaagattgaaactcaaaggaattgacgggggcccgcacaagcagtggagtatgtggtttaattcgaagcaacgcgaagaaccttaccaggacttgacatcccacgcatagcctagagataggtgaagtcctacgggacgtggagacaggtggtgcatggttgtcgtcagctcgtgtcgtgagatgttgggttaagtcccgcaacgagcgcaacccttactgtcagttaccatcattaagttggggactctggcaggactgccggtgacaaatcggaggaaggtggggacgacgtcaaatcatcatgccccttatgtcctgggctacacacgtactacaatggctgtaaacagagtgaagcaaagcagtgatgtggagcaaaacacaaaaagcagtctcagttcagattgtaggctgaaactcgcctatatgaagtcggaattgctagtaatcgcagatcagcatgctgcggtgaatacgttcccgggccttgtacacaccgcccgtcacaccatgagagtcgataacacccgaagcctgtagcttaaccttagggagagc

SEQ ID NO: 86 - DNA, 1527 bp, synthetic construct
  misc_feature 1..1527: note = Synthetic: FBI00151
  source 1..1527: mol_type = other DNA
tggagagtttgatcctggctcaggatgaacgctggcggcgtgcctaacacatgcaagtcgaacgaagcgactggaggaagttttcggatggaatccggttgactgagtggcggacgggtgagtaacgcgtggataacctgcctcacactgggggataacagttagaaatggctgctaataccgcataagcgcacagtaccgcatggtacggtgtgaaaaacccaggtggtgtgagatggatccgcgtctgattagccagttggcggggtaacggcccaccaaagcgacgatcagtagccgacctgagagggtgaccggccacattgggactgagacacggcccaaactcctacgggaggcagcagtggggaatattgcacaatgggcggaagcctgatgcagcgacgccgcgtgagtgaagaagtatctcggtatgtaaagctctatcagcagggaagaaaatgacggtacctgactaagaagccccggctaactacgtgccagcagccgcggtaatacgtagggggcaagcgttatccggatttactgggtgtaaagggagcgtagacggcgacgcaagtctggagtgaaagcccggggcccaaccccgggactgctttggaaactgtgctgctggagtgcaggagaggtaagtggaattcctagtgtagcggtgaaatgcgtagatattaggaggaacaccagtggcgaaggcggcttactggactgtaactgacgttgaggctcgaaagcgtggggagcaaacaggattagataccctggtagtccacgccgtaaacgatgaatgctaggtgtcggggggcaaagcccttcggtgccgccgctaacgcaataagcattccacctggggagtacgttcgcaagaatgaaactcaaaggaattgacggggacccgcacaagcggtggagcatgtggtttaattcgaagcaacgcgaagaaccttaccaagtcttgacatccccctgaccggymcgtaacggkgcccttccttcgggacaggggagacaggtggtgcatggttgtcgtcagctcgtgtcgtgagatgttgggttaagtcccgcaacgagcgcaacccttatccttagtagccagcacgtsrtggtgggcactctagggagactgccagggataacctggaggaaggtggggatgacgtcaaatcatcatgccccttatgatttgggctacacacgtgctacaatggcgtaaacaaagggaggcgaccctgcgaaggcaagcaaatcccaaaaataacgtcccagttcggactgtagtctgcaacccgactacacgaagctggaatcgctagtaatcgcgratcagaatgycgcggtgaatacgttcccgggtcttgtacacaccgcccgtcacaccatgggagtcagcaacgcccgaagtcagtgacccaacctkaamrggagggagctgccgaaggcggggcaggtaactggggtgaagtcgtaacaaggtagccgtatcggaaggtgcggytggatcacctcctt

SEQ ID NO: 87 - DNA, 1429 bp, synthetic construct
  misc_feature 1..1429: note = Synthetic: FBI00152
  source 1..1429: mol_type = other DNA
tcgaacgaaaagagggaaagagcttgctctttncngaattgagtggcaaacgggtgagtaacacgtaaacaacctgccttcaggatggggacaacagacggaaacgactgctaataccgaataagttccaagagccgcatggcccatggaagaaaaggtggcctctacctgtaagctatcgcctgaagaggggtttgcgtctgattagctggttggaggggtaacggcccaccaaggcgacgatcagtagccggtctgagaggatgaacggccacactggaactgagacacggtccagactcctacgggaggcagcagtggggaatcttccgcaatgggcgaaagcctgacggagcaacgccgcgtgagtgatgacggccttcgggttgtaaaactctgtgatccgggacgaaaaggcagagtgcgaagaacaaactgcattgacggtaccggaaaagcaagccacggctaactacgtgccagcagccgcggtaatacgtaggtggcaagcgttgtccggaattattgggcgtaaagcgcgcgcaggcggcttcccaagtccctcttaaaagtgcggggcttaaccccgtgatgggaaggaaactgggaagctggagtatcggagaggaaagtggaattcctagtgtagcggtgaaatgcgtagagattaggaagaacaccggtggcgaaggcgactttctggacgaaaactgacgctgaggcgcgaaagcgtggggagcaaacaggattagataccctggtagtccacgccgtaaacgatggatactaggtgtaggaggtatcgacccctcctgtgccggagttaacgcaataagtatcccgcctgggaagtacgatcgcaagattaaaactcaaaggaattgacgggggcccgcacaagcggtggagtatgtggtttaattcgacgcaacgcgaagaaccttaccaggtcttgacattgatcgcgatctgcagaaatgcggagttcttcttcggaagacgagaaaacaggtggtgcacggctgtcgtcagctcgtgtcgtgagatgttgggttaagtcccgcaacgagcgcaacccctatcatttgttgccagcacgtaaaggtggggactcaaatgagaccgccgcggacaacgcggaggaaggcggggacgacgtcaagtcatcatgccccttatgacctgggctacacacgtactacaatgggtgtcaacaaagagaagcgaacccgcgagggagagcaaagctcaaaaacacacccccagttcagatcgcaggctgcaacccgcctgcgtgaagtaggaatcgctagtaatcgcgggtcagcataccgcggtgaatacgttcccgggccttgtacacaccgcccgtcacactatgagagtcagaaacacccgaagccggtgaggtaaccgcaaggagccnncc

SEQ ID NO: 88 - DNA, 1396 bp, synthetic construct
  misc_feature 1..1396: note = Synthetic: FBI00159
  source 1..1396: mol_type = other DNA
tgcagtcgaacgaagttagacagaggaagttttcggatggaatcggtataacttagtggcggacgggtgagtaacgcgtgggaaacctgccctgtaccgggggataacacttagaaataggtgctaataccgcataagcgcacggaaccgcatggttgtgtgtgaaaaactccggtggtacaggatggtcccgcgtctgattagccagttggcagggtaacggcctaccaaagcgacgatcagtagccggcctgagagggtgaacggccacattgggactgagacacggcccaaactcctacgggaggcagcagtggggaatattgcacaatgggggaaaccctgatgcagcgacgccgcgtgagtgaagaagtatttcggtatgtaaagctctatcagcagggaagaaaatgacggtacctgactaagaagccccggctaactacgtgccagcagccgcggtaatacgtagggggcaagcgttatccggatttactgggtgtaaagggagcgtagacggcatggcaagccagatgtgaaaacccagggctcaaccttgggattgcatttggaactgccaggctggagtgcaggagaggtaagcggaattcctagtgtagcggtgaaatgcgtagatattaggaggaacaccagtggcgaaggcggcttactggactgtaactgacgttgaggctcgaaagcgtggggagcaaacaggattagataccctggtagtccacgcggtaaacgatgattgctaggtgtaggtgggtatggacccatcggtgccgcagctaacgcaataagcaatccacctggggagtacgttcgcaagaatgaaactcaaaggaattgacggggacccgcacaagcggtggagcatgtggtttaattcgaagcaacgcgaagaaccttaccaagtcttgacatccccttgaagatcttgtaaagaggcattcccttcggggcaagggagacaggtggtgcatggttgtcgtcagctcgtgtcgtgagatgttgggttaagtcccgcaacgagcgcaacccttattcttagtagccagcaggtaaagctgggcactctaaggagactgccggggataacccggaggaaggcggggatgacgtcaaatcatcatgccccttatgatttgggctacacacgtgctacaatggcgtaaacaaagggaagcgagacagtgatgtggagcaaatcccagaaataacgtctcagttcggattgtagtctgcaactcgactacatgaagctggaatcgctagtaatcgcgaatcagcatgtcgcggtgaatacgttcccgggtcttgtacacaccgcccgtcacaccatgggagttggaaatgcccgaagtctgtgacctaaccgaaagggannagc

SEQ ID NO: 89 - DNA, 1395 bp, synthetic construct
  misc_feature 1..1395: note = Synthetic: FBI00162
  source 1..1395: mol_type = other DNA
tgcagtcgaacgggnnccnggcagcttgctgcctggtgagagtggcgaacgggtgagtaatgcgtgaccgacctgccccatacaccggaatagctcctggaaacgggtggtaatgccggatgctccgactcctcgcatggggtgtcgggaaagattatatcggtatgggatggggtcgcgtcctatcaggtagtcggcggggtaacggcccaccgagcctacgacgggtagccggcctgagagggcgaccggccacattgggactgagatacggcccagactcctacgggaggcagcagtggggaatattgcacaatgggcgcaagcctgatgcagcgacgccgcgtgcgggatgacggccttcgggttgtaaaccgcttttgatcgggagcaagccttcgggtgagtgtacctttcgaataagcaccggctaactacgtgccagcagccgcggtaatacgtagggtgcaagcgttatccggaattattgggcgtaaagggctcgtaggcggtttcgtcgcgtccggtgtgaaagtccatcgcttaacggtggatctgcgccgggtacgggcgggctggagtgcggtaggggagactggaattccccggtgtaacggtggaatgtgtagatatcgggaagaacaccaatggcgaaggcaggtctctgggccgttactgacgctgaggagcgaaagcgtggggagcgaacaggattagataccctggtagtccacgccgtaaacggtggatgctggatgtgggggcccgttccacgggttccgtgtcggagctaacgcgttaagcatcccgcctggggagtacggccgcaaggctaaaactcaaagaaattgacgggggcccgcacaagcggcggagcatgcggattaattcgatgcaacgcgaagaaccttacctgggcttgacatgttcccgacagccgtagagatgcggcctcccttcggggcgggttcacaggtggtgcatggtcgtcgtcagctcgtgtcgtgagatgttgggttaagtcccgcaacgagcgcaaccctcgccctgtgttgccagcacgtcatggtgggaactcacgggggaccgccggggtcaactcggaggaaggtggggatgacgtcagatcatcatgccccttacgtccagggcttcacgcatgctacaatggccggtacaacgggatgcgacatggcgacatggagcggatccctgaaaaccggtctcagttcggattggagtctgcaacccgactccatgaaggcggagtcgctagtaatcgcggatcagcaacgccgcggtgaatgcgttcccgggccttgtacacaccgcccgtcaagtcatgaaagtgggtagcacccgaagccggtggcctaaccccttgtggganggagc

SEQ ID NO: 90 - DNA, 1384 bp, synthetic construct
  misc_feature 1..1384: note = Synthetic: FBI00165
  source 1..1384: mol_type = other DNA
tcgaggggcagcatggtcttagcttgctaaggccgatggcgaccggcgcacgggtgagtaacgcgtatccaacctgccttacactcttggacagccttctgaaagggagattaatacaagatgttatcatgagtaagcattttcgcatgattaaaggtttaccggtgtaagatggggatgcgttccattagatagtaggcggggtaacggcccacctagtcttcgatggataggggttctgagaggaaggtcccccacattggaactgagacacggtccaaactcctacgggaggcagcagtgaggaatattggtcaatggacgagagtctgaaccagccaagtagcgtgaaggatgaaggttctatggattgtaaacttcttttatacgggaataaacgaatccacgcgtggatttttgcatgtaccgtatgaataaggatcggctaactccgtgccagcagccgcggtaatacggaggatccgagcgttatccggatttattgggtttaaagggagcgtagatgggttgttaagtcagttgtgaaagtttgcggctcaaccgtaaaattgcaattgatactggcagtcttgagtacagttgaggtaggcggaattcgtggtgtagcggtgaaatgcttagatatcacgaagaactccgattgcgaaggcagcttactaacctgtaactgacattgatgctcgaaagtgtgggtatcaaacaggattagataccctggtagtccacacggtaaacgatgaatactcgctgtaggcgatatacggtctgcggccaagcgaaagcattaagtattccacctggggagtacgccggcaacggtgaaactcaaaggaattgacgggggcccgcacaagcggaggaacatgtggtttaattcgatgatacgcgaggaaccttacccgggcttaaattgcaaccgaatatggcggaaacgccatagctagcaatagcggttgtgaaggtgctgcatggttgtcgtcagctcgtgccgtgaggtgtcggcttaagtgccataacgagcgcaacccttgccgatagttactaacaggttatgctgaggactctgtcgggactgccatcgtaagatgtgaggaaggtggggatgacgtcaaatcagcacggcccttacgtccggggctacacacgtgttacaatggggggtacagagggctgctaccacgcaagtggatgccaatcccaaaaacctctctcagttcggattgaagtctgcaacccgacttcatgaagctggattcgctagtaatcgcgcatcagccacggcgcggtgaatacgttcccgggccttgtacacaccgcccgtcaagccatgggagccgggggtacctgaagtgcgtaaccgcaa

SEQ ID NO: 91 - DNA, 1391 bp, synthetic construct
  misc_feature 1..1391: note = Synthetic: FBI00167
  source 1..1391: mol_type = other DNA
tcgagcgaagcacttnnggtnngattcttcggatgannnnntttgtgactgagcggcggacgggtgagtaacgcgtgggtaacctgcctcatacagggggataacagttagaaatgactgctaataccgcataagaccacggtaccgcatggtacagtggtaaaaactccggtggtatgagatggacccgcgtctgattaggtagttggtggggtaacggcctaccaagccgacgatcagtagccgacctgagagggtgaccggccacattgggactgagacacggcccagactcctacgggaggcagcagtggggaatattgcacaatggaggaaactctgatgcagcgacgccgcgtgaaggatgaagtatttcggtatgtaaacttctatcagcagggaagaaaatgacggtacctgactaagaagccccggctaactacgtgccagcagccgcggtaatacgtagggggcaagcgttatccggatttactgggtgtaaagggagcgtagacggcacggcaagccagatgtgaaagcccggggctcaaccccgggactgcatttggaactgctgagctagagtgtcggagaggcaagtggaattcctagtgtagcggtgaaatgcgtagatattaggaggaacaccagtggcgaaggcggcttgctggacgatgactgacgttgaggctcgaaagcgtggggagcaaacaggattagataccctggtagtccacgccgtaaacgatgactgctaggtgtcgggtggcaaagccattcggtgccgcagctaacgcaataagcagtccacctggggagtacgttcgcaagaatgaaactcaaaggaattgacggggacccgcacaagcggtggagcatgtggtttaattcgaagcaacgcgaagaaccttacctgatcttgacatcccgatgaccgcttcgtaatggaagcttttcttcggaacatcggtgacaggtggtgcatggttgtcgtcagctcgtgtcgtgagatgttgggttaagtcccgcaacgagcgcaacccctatcttcagtagccagcaggtantgttgggcactctggagagactgccagggataacctggaggaaggtggggatgacgtcaaatcatcatgccccttatgaccagggctacacacgtgctacaatggcgtaaacaaagagaagcgaactcgcgagggtaagcaaatctcaaaaataacgtctcagttcggattgtagtctgcaactcgactacatgaagctggaatcgctagtaatcgcagatcagaatgctgcggtgaatacgttcccgggtcttgtacacaccgcccgtcacaccatgggagtcagtaacgcccgaagtcagtgacccaaccgtaaggagggag

SEQ ID NO: 92 - DNA, 1374 bp, synthetic construct
  misc_feature 1..1374: note = Synthetic: FBI00170
  source 1..1374: mol_type = other DNA
tgcagtcnnnnatgaaaccgccctcgggcggacatgaagtggcgaacgggtgagtaacacgtgaccaacctgcccccctctccgggacaaccttgggaaaccgaggctaataccggatactccctcccctgctcctgcaggggtcgggaaagcccaggcggagggggatggggtcgcggcccattaggtagtaggcggggtaacggcccacctagcccgcgatgggtagccgggttgagagaccgaccggccacattgggactgagatacggcccagactcctacgggaggcagcagtggggaattttgcgcaatgggggaaaccctgacgcagcaacgccgcgtgcgggacgacggccttcgggttgtaaaccgctttcagcagggaagaaattcgacggtacctgcagaagaagctccggctaactacgtgccagcagccgcggtaatacgtagggagcgagcgttatccggaatcattgggcgtaaagagcgcgtaggcggcctctcaagcgggatctctaatccgagggctcaaccccccggccggatcccgaactgggaggctcgagttcggtagaggcaggcggaattcccggtgtagcggtggaatgcgcagatatcgggaagaacaccgatggcgaaggcagcctgctgggccgcaactgacgctgaggcgcgaaagctaggggagcgaacaggattagataccctggtagtcctagccgtaaacgatggatactaggtgtgggggggctccgccctccgtgccgcagcccaacgcattaagtatcccgcctggggagtacggccgcaaggctaaaactcaaaggaattgacgggggcccgcacaagcagcggagcatgtggcttaattcgaagcaacgcgaagaaccttaccagggcttgacatggacgtgaagccggggaaacccggtggccgagaggagcgtccgcaggtggtgcatggctgtcgtcagctcgtgtcgtgagatgttgggttaagtcccgcaacgagcgcaacccctgccccatgttgccagcattaggttggggactcatgggggactgccggcgtcaagccggaggaaggtggggacgacgtcaagtcatcatgccctttatgccctgggctgcacacgtgctacaatggccggtacaacgggctgcgagaccgcgaggtcgagcgaatccctcaaagccggccccagttcggatcggaggctgcaacccgcctccgtgaagtcggagttgctagtaatcgcggatcagcatgccgcggtgaatacgttcccgggccttgtacacaccgcccgtcacaccacccgagtcgtctgcacccgaagccgccggccgaacccgcaaggggcggag

SEQ ID NO: 93 - DNA, 1396 bp, synthetic construct
  misc_feature 1..1396: note = Synthetic: FBI00171
  source 1..1396: mol_type = other DNA
tcgaacgtgaaagtccttcgggacgagtaaagtggcgcacgggtgagtaacgcgtggataatctacccttaagatggggataacggctggaaacggtcgctaataccgaatacgctcccgattttatcattggggggaaagatggcctctgcttgcaagctatcgcttaaggatgagtccgcgtcccattagctagttggcggggtaacggcccaccaaggcaacgatgggtagccggtctgagaggatgaccggccacactggaactggaacacggtccagactcctacgggaggcagcagtggggaatattgcgcaatgggcgaaagcctgacgcagcgacgccgcgtgagggatgaaggttctcggatcgtaaacctctgtcaggggggaagaaaccccctcgtgtgaataatgcgagggcttgacggtacccccaaaggaagcaccggctaactccgtgccagcagccgcggtaatacggagggtgcaagcgttaatcggaatcactgggcgtaaagcgcacgtaggcggcttggtaagtcaggggtgaaatcccacagcccaactgtggaactgcctttgatactgccaggcttgagtaccggagagggtggcggaattccaggtgtaggagtgaaatccgtagatatctggaggaacaccggtggcgaaggcggccacctggacggtaactgacgctgaggtgcgaaagcgtgggtagcaaacaggattagataccctggtagtccacgctgtaaacgatgggtgctgggtgctgggatgtatgtctcggtgccgtagctaacgcgataagcaccccgcctggggagtacggtcgcaaggctgaaactcaaagaaattgacggggggcccgcacaagcggtggagtatgtggtttaattcgatgcaacgcgaagaaccttacccaggcttgacatctagggaacccttcggaaatgaaggggtgcccttcggggagccctaagacaggtgctgcatggctgtcgtcagctcgtgccgtgaggtgttgggttaagtcccgcaacgagcgcaacccctatcttcagttgccagcaggtaaggctgggcactctggagagaccgccccggtcaacggggaggaaggtggggacgacgtcaagtcatcatggcccttacgcctggggctacacacgtactacaatggcgcgcacaaagggtagcgagaccgcgaggtggagccaatcccaaaaaacgcgtcccagtccggattggagtctgcaactcgactccatgaagtcggaatcgctagtaattcgagatcagcatgctcgggtgaatgcgttcccgggccttgtacacaccgcccgtcacaccacgaaagtcggttttacccgaagccggtnagctaact

SEQ ID NO: 94 - DNA, 1389 bp, synthetic construct
  misc_feature 1..1389: note = Synthetic: FBI00174
  source 1..1389: mol_type = other DNA
gtcgaacgaagcatttnnnnacggattnnttcggnntgaagnnnnttatgactgagtggcggacgggtgagtaacgcgtgggtaacctgcctcacacagggggatagcagttggaaacggctgataataccgcataagcgcacagtaccgcatggtacagtgtgaaaaactccggtggtgtgagatggacccgcgtctgattagcttgttggcggggtaacggcccaccaaggcaacgatcagtagccgacctgagagggtgaccggccacattgggactgagacacggcccagactcctacgggaggcagcagtggggaatattgcacaatggaggaaactctgatgcagcgacgccgcgtgagtgaagaagtaattcgttatgtaaagctctatcagcagggaagatagtgacggtacctgactaagaagctccggctaaatacgtgccagcagccgcggtaatacgtatggagcaagcgttatccggatttactgggtgtaaagggagtgtaggtggcatcacaagtcagaagtgaaagcccggggctcaaccccgggactgcttttgaaactgtggagctggagtgcaggagaggcaagtggaattcctagtgtagcggtgaaatgcgtagatattaggaggaacaccagtggcgaaggcggcttgctggactgtaactgacactgaggctcgaaagcgtggggagcaaacaggattagataccctggtagtccacgccgtaaacgatgaatactaggtgtcggggctcataagagcttcggtgccgcagcaaacgcaataagtattccacctggggagtacgttcgcaagaatgaaactcaaaggaattgacggggacccgcacaagcggtggagcatgtggtttaattcgaagcaacgcgaagaaccttaccaagtcttgacatcctcttgaccggtcagtaatgtgaccttttcttcggaacaagagtgacaggtggtgcatggttgtcgtcagctcgtgtcgtgagatgttgggttaagtcccgcaacgagcgcaacccctattcttagtagccagcatataaggtgggcactctaggaagactgccagggataacctggaggaaggtggggatgacgtcaaatcatcatgccccttatgacttgggctacacacgtgctacaatggcgtaaacaaagtgaagcgagagtgtgagcttaagcaaatcacaaaaataacgtctcagttcggattgtagtctgcaactcgactacatgaagctggaatcgctagtaatcgcgaatcagaatgtcgcggtgaatacgttcccgggtcttgtacacaccgcccgtcacaccatgggagtcggaaatgcccgaagtcggtgacctaacgaaagaag

SEQ ID NO: 95 - DNA, 1386 bp, synthetic construct
  misc_feature 1..1386: note = Synthetic: FBI00175
  source 1..1386: mol_type = other DNA
gcagtcgnncgagaggaaggnaagcttgcttttctaaatctagtggcgaacgggtgagtaacacgtaggtaacctgcccatgtgcccgggataacttctggaaacggatgctaaaaccggataggtagcagacaagcatttgactgctattaaagtggctaaggccatgaacatggatggacctgcggtgcattagctagttggtgaggtaacggcccaccaaggcgacgatgcatagccggcctgagagggcggacggccacattgggactgagacacggcccaaactcctacgggaggcagcagtagggaattttcgtcaatggggggaaccctgaacgagcaatgccgcgtgagtgaggaaggtcttcggatcgtaaagctctgttgtaagagaaaaacggcactcatagggaatgatgagtgagtgatggtatcttaccagaaagtcacggctaactacgtgccagcagccgcggtaatacgtaggtggcgagcgttatccggaatgattgggcgtaaagggtgcgtaggtggcagatcaagtctggagtaaaaggtatgggctcaacccgtacttgctctggaaactgatcagctagagaacagaagaggacggcggaactccatgtgtagcggtaaaatgcgtagatatatggaagaacaccggtggcgaaggcggccgtctggtctggattctgacactgaagcacgaaagcgtggggagcaaataggattagataccctagtagtccacgccgtaaacgatgagaactaggtgttgggggaataactcagtgccgcagttaacgcaataagttctccgcctggggagtatgcacgcaagtgtgaaactcaaaggaattgacgggggcccgcacaagcggtggagtatgtggtttaattcgaagcaacgcgaagaaccttaccaggccttgacatcccttgcagaggtatagagatatatccgaggttatcaaggagacaggtggtgcatggttgtcgtcagctcgtgtcgtgagatgttgggttaagtcccgcaacgagcgcaacccttgtgatatgttactaacattgagttgaggactcatatcagactgccggtgataaaccggaggaaggtggggatgacgtcaaatcatcatgccccttatggcctgggctacacacgtactacaatggcgtctacaaagagcggcaagcctgtgaaggcaagcgaatctcataaaggacgtctcagttcggattgaagtctgcaacccgacttcatgaagctggaatcgctagtaatcgcgaatcagcatgtcgcggtgaatacgttctcgggccttgtacacaccgcccgtcaaaccatgggagttggtaatgcccgaagccggtngnnta

SEQ ID NO: 96 - DNA, 1386 bp, synthetic construct
  misc_feature 1..1386: note = Synthetic: FBI00176
  source 1..1386: mol_type = other DNA
gnnagtcgancggagctgttttctctgaagttttcggatggaagagagttcagcttagtggcgaacgggtgagtaacacgtgagcaacctgcctttcagtgggggacaacatttggaaacgaatgctaataccgcataagaccacagtgtcgcatggcacaggggtcaaaggatttatccgctgaaagatgggctcgcgtccgattagctagatggtgaggtaacggcccaccatggcgacgatcggtagccggactgagaggttgaacggccacattgggactgagacacggcccagactcctacgggaggcagcagtggggaatattgcacaatgggggaaaccctgatgcagcgacgccgcgtggaggaagaaggtcttcggattgtaaactcctgtcccaggggacgataatgacggtaccctgggaggaagcaccggctaactacgtgccagcagccgcggtaaaacgtagggtgcaagcgttgtccggaattactgggtgtaaagggagcgcaggcggattggcaagttgggagtgaaatctatgggctcaacccataaattgctttcaaaactgtcagtcttgagtggtgtagaggtaggcggaattcccggtgtagcggtggaatgcgtagatatcgggaggaacaccagtggcgaaggcggcctactgggcactaactgacgctgaggctcgaaagcatgggtagcaaacaggattagataccctggtagtccatgccgtaaacgatgattactaggtgtgggaggattgaccccttccgtgccgcagttaacacaataagtaatccacctgggggagtacgaccgcaaggttgaaactcaaaggaattgacgggggcccgcacaagcagtgggagtatgtggtttaattcgaagcaacgcgaagaaccttaccaggtcttgacatcggatgcatacctaagagattagggaagtccttcgggacatccagacaggtggtgcatggttgtcgtcagctcgtgtcgtgagatgttgggttaagtcccgcaacgagcgcaacccttatcgttagttactacgcaagaggactctagcgagactgccgttgacaaaacggaggaaggtggggatgacgtcaaatcatcatgccctttatgacctgggctacacacgtactacaatggctattaacagagagaagcgataccgcgaggtggagcaaacctcacaaaaatagtctcagttcggatcgcaggctgcaacccgcctgcgtgaagccggaattgctagtaatcgcggatcagcatgccgcggtgaatacgttcccgggccttgtacacaccgcccgtcacaccatgagagccggggggacccgaagtcggtagtctaaccgcaag

SEQ ID NO: 97 - DNA, 1359 bp, synthetic construct
  misc_feature 1..1359: note = Synthetic: FBI00177
  source 1..1359: mol_type = other DNA
agaagcttgcttctcnccggcgacgagtggcgaacgggtgagtaatacatcggaacgtgtccgctcgtgggggacaaccagccgaaaggttggctaataccgcatgagttctacggaagaaagagggggacccgcaagggcctctcgcgagcggagcggccgatgactgattagccggttggtgaggtaacggctcaccaaagcaacgatcagtagctggtctgagaggacgaccagccacactgggactgagacacggcccagactcctacgggaggcagcagtggggaattttggacaatgggcgcaagcctgatccagctattccgcgtgtgggatgaaggccctcgggttgtaaaccacttttgtagagaacgaaaagacaccttcgaataaagggtgttgctgacggtactctaagaataagcaccggctaactacgtgccagcagccgcggtaatacgtagggtgcgagcgttaatcggaattactgggcgtaaagggtgcgcaggcggttgagtaagacagatgtgaaatccccgagcttaactcgggaatggcatatgtgactgctcgactagagtgtgtcagagggaggtggaattccacgtgtagcagtgaaatgcgtagatatgtggaagaacaccgatggcgaaggcagcctcctgggacataactgacgctcaggcacgaaagcgtggggagcaaacaggattagataccctggtagtccacgccctaaacgatgttaactagttgttgggaagtaaaattctcagtaacgcagccaacgcgagaagttaaccgcctgggaagtacggtcgcaagactaaaactcaaaggaattgacggggacccgcacaagcggtggatgatgtggattaattcgatgcaacgcgaaaaaccttacctacccttgacatgtcaggaagctcttgtaatgagagcgtgcccgcaagggagcctgaacacaggtgctgcatggctgtcgtcagctcgtgtcgtgagatgttgggttaagtcccgcaacgagcgcaacccttgtcactagttgctacgaaagggcactctagtgagactgccggtgacaaaccggaggaaggtggggatgacgtcaagtcctcatggcccttatgggtagggcttcacacgtcatacaatggtcggaacagagggcagcgaagtcgtgagacggagccaatcccagaaaaccgatcgtagtccggattgcagtctgcaactcgactgcatgaagtcggaatcgctagtaatcgcggatcagcatgccgcggtgaatacgttcccgggtcttgtacacaccgcccgtcaaacaatgggagtggtgtttaccagaagtcg

SEQ ID NO: 98 - DNA, 1391 bp, synthetic construct
  misc_feature 1..1391: note = Synthetic: FBI00180
  source 1..1391: mol_type = other DNA
tgcnagtcngaggggcagcattcgggtagcaatacctgagatggcgaccggcgcacgggtgcgtaacgcgtatgcaacctacccataacaggggcataacactgagaaattggtactaattccccataacattcgaagaggcatcttttcgggttgaaaactccggtggttatggatgggcatgcgttgtattagctggttggtgaggtaacggctcaccaaggcgacgatacatagggggactgagaggttaaccccccacattggtactgagacacggaccaaactcctacgggaggcagcagtgaggaatattggtcaatggacgcaagtctgaaccagccatgccgcgtgcaggaagacggctctatgagttgtaaactgcttttgtacgagggtaaactcagatacgtgtatctgactgaaagtatcgtacgaataaggatcggctaactccgtgccagcagccgcggtaatacggaggattcaagcgttatccggatttattgggtttaaagggtgcgtaggcggtctgataagttagaggtgaaatcccggggcttaactccggaattgcctctgatactgttgggctagagagtagttgcggtaggcggaatgtatggtgtagcggtgaaatgcttagagatcatacagaacaccgattgcgaaggcagcttaccaaactatatctgacgttgaggcacgaaagcgtggggagcaaacaggattagataccctggtagtccacgcagtaaacgatgataactcgctgtcggcgatacacagtcggtggctaagcgaaagcgataagttatccacctggggagtacgttcgcaagaatgaaactcaaaggaattgacgggggcccgcacaagcggaggaacatgtggtttaattcgatgatacgcgaggaaccttacccgggcttgaaagttagcgacgattctggaaacaggatttcccttcggggcgcgaaactaggtgctgcatggttgtcgtcagctcgtgccgtgaggtgtcgggttaagtcccataacgagcgcaacccctaccgttagttgccatcaggtcaagctgggcactctggcgggactgccggtgtaagccgagaggaaggtggggatgacgtcaaatcagcacggcccttacgtccggggctacacacgtgttacaatggtaggtacagagggcagctaccctgcgaagggatgcgaatctcgaaagcctatctcagttcggattggaggctgaaacccgcctccatgaagttggattcgctagtaatcgcgcatcagccatggcgcggtgaatacgttcccgggccttgtacacaccgcccgtcaagccatggaagctgggggtgcctgaagttcgtgaccgncaagga

SEQ ID NO: 99 - DNA, 1392 bp, synthetic construct
  misc_feature 1..1392: note = Synthetic: FBI00182
  source 1..1392: mol_type = other DNA
angtcgaggggcagcatgaacttagctntgctaagtnttgatggcgaccggcgcacgggtgagtaacacgtatccaaccttccgtttactcagggatagcctttcgaaagaaagattaatacctgatagtatggtgagattgcatgatgacaccattaaagatttattggtaaacgatggggatgcgttccattaggtagtaggcggggtaacggcccacctagccggcgatggataggggttctgagaggaaggtcccccacattggaactgagacacggtccaaactcctacgggaggcagcagtgaggaatattggtcaatgggcgagagcctgaaccagccaagtagcgtgaaggatgaaggtcctacggattgtaaacttcttttatacgggaataaagtttcctacgtgtaggattttgtatgtaccgtatgaataagcatcggctaactccgtgccagcagccgcggtaatacggaggatgcgagcgttatccggatttattgggtttaaagggagcgcagacgggagattaagtcagttgtgaaagtttgcggctcaaccgtaaaattgcagttgatactggtttccttgagtgcagttgaggcaggcggaattcgtggtgtagcggtgaaatgcttagatatcacgaagaaccccgattgcgaaggcagcttgctaaactgtaactgacgttcatgctcgaaagtgtgggtatcaaacaggattagataccctggtagtccacacggtaaacgatggatactcgctgttggcgatatactgtcagcggccaagcgaaagcattaagtatcccacctggggagtacgccggcaacggtgaaactcaaaggaattgacgggggcccgcacaagcggaggaacatgtggtttaattcgatgatacgcgaggaaccttacccgggcttaaattgcagacgaattacgaggaaacttgtaagccgcaaggcgtctgtgaaggtgctgcatggttgtcgtcagctcgtgccgtgaggtgtcggcttaagtgccataacgagcgcaaccctcgtggtcagttactaacaggttaagctgaggactctggccagactgccatcgtaagatgtgaggaaggtggggatgacgtcaaatcagcacggcccttacgtccggggctacacacgtgttacaatgggaggtacagaaggccgctacccggcaacgggatgccaatccccaaaacctctctcagttcggactggagtctgcaacccgactccacgaagctggattcgctagtaatcgcgcatcagccacggcgcggtgaatacgttcccgggccttgtacacaccgcccgtcaagccatgaaagccgggggtacctgaagtgcgtaaccgcaaggagc

SEQ ID NO: 100 - DNA, 1386 bp, synthetic construct
  misc_feature 1..1386: note = Synthetic: FBI00184
  source 1..1386: mol_type = other DNA
gcagtcgaggggcatcatgcttatagcaatataagtgatggcgaccggcgcacgggtgagtaacacgtatccaacctgccgataactcggggatagcctttcgaaagaaagattaatacccgatggcataatagaaccgcacggtttgattattaaagaatttcggttatcgatggggatgcgttccattaggcagttggtggggtaacggcccaccaaaccttcgatggataggggttctgagaggaaggtcccccacattggaactgagacacggtccaaactcctacgggaggcagcagtgaggaatattggtcaatggacgagagtctgaaccagccaagtagcgtgaaggatgactgccctatgggttgtaaacttcttttatatgggaataaagtggtccacgtgtggatttttgtatgtaccatatgaataaggatcggctaactccgtgccagcagccgcggtaatacggaggatccgagcgttatccggatttattgggtttaaagggagcgtaggtggacagttaagtcagttgtgaaagtttgcggctcaaccgtaaaattgcagttgatactggctgtcttgagtacagtagaggtgggcggaattcgtggtgtagcggtgaaatgcttagatatcacgaagaactccgattgcgaaggcagctcactggactgcaactgacactgatgctcgaaagtgtgggtatcaaacaggattagataccctggtagtccacacagtaaacgatgaatactcgctgtttgcgatatacagtaagcggccaagcgaaagcattaagtattccacctggggagtacgccggcaacggtgaaactcaaaggaattgacgggggcccgcacaagcggaggaacatgtggtttaattcgatgatacgcgaggaaccttacccgggcttaaattgcatttgaatatattggaaacagtatagtcgtaagacaaatgtgaaggtgctgcatggttgtcgtcagctcgtgccgtgaggtgtcggcttaagtgccataacgagcgcaacccttatctttagttactaacaggtcatgctgaggactctagagagactgccgtcgtaagatgtgaggaaggtggggatgacgtcaaatcagcacggcccttacgtccggggctacacacgtgttacaatggggggtacagaaggcagctacctggtgacaggatgctaatcccaaaagcctctctcagttcggatcgaagtctgcaacccgacttcgtgaagctggattcgctagtaatcgcgcatcagccatggcgcggtgaatacgttcccgggccttgtacacaccgcccgtcaagccatgaaagccgggggtacctgaagtacgtaaccgcaanng

SEQ ID NO: 101 - DNA, 1386 bp, synthetic construct
  misc_feature 1..1386: note = Synthetic: FBI00189
  source 1..1386: mol_type = other DNA
gaggggcagcattttagtttgcttgcaaactgaagatggcgaccggcgcacgggtgagtaacacgtatccaacctgccgataactccggaatagcctttcgaaagaaagattaataccggatagcatacgaatatcgcatgatatttttattaaagaatttcggttatcgatggggatgcgttccattagtttgttggcggggtaacggcccaccaagactacgatggataggggttctgagaggaaggtcccccacattggaactgagacacggtccaaactcctacgggaggcagcagtgaggaatattggtcaatgggcgagagcctgaaccagccaagtagcgtgaaggatgaaggctctatgggtcgtaaacttcttttatatgggaataaagttttccacgtgtggaattttgtatgtaccatatgaataaggatcggctaactccgtgccagcagccgcggtaatacggaggatccgagcgttatccggatttattgggtttaaagggagcgtaggtggattgttaagtcagttgtgaaagtttgcggctcaaccgtaaaattgcagttgaaactggcagtcttgagtacagtagaggtgggcggaattcgtggtgtagcggtgaaatgcttagatatcacgaagaactccgattgcgaaggcagctcactagactgtcactgacactgatgctcgaaagtgtgggtatcaaacaggattagataccctggtagtccacacagtaaacgatgaatactcgctgtttgcgatatacagtaagcggccaagcgaaagcattaagtattccacctggggagtacgccggcaacggtgaaactcaaaggaattgacgggggcccgcacaagcggaggaacatgtggtttaattcgatgatacgcgaggaaccttacccgggcttaaattgcaacagaatatattggaaacagtatagccgtaaggctgttgtgaaggtgctgcatggttgtcgtcagctcgtgccgtgaggtgtcggcttaagtgccataacgagcgcaacccttatctttagttactaacaggtcatgctgaggactctagagagactgccgtcgtaagangtgaggaaggtggggatgacgtcaaatcagcacggcccttacgtccggggctacacacgtgttacaatggggggtacagaaggcggctacctggtgacaggatgctaatcccaaaaacctctctcagttcggatcgaagtctgcaacccgacttcgtgaagctggattcgctagtaatcgcgcatcagccatggcgcggtgaatacgttcccgggccttgtacacaccgcccgtcaagccatgaaagccgggggtacctgaagtacgtaaccgcaaggagc

SEQ ID NO: 102 - DNA, 1390 bp, synthetic construct
  misc_feature 1..1390: note = Synthetic: FBI00190
  source 1..1390: mol_type = other DNA
agtcgaggggcagcattttagtttgcttgcaaactaaagatggcgaccggcgcacgggtgagtaacacgtatccaacctgccgataactcagggatagcctttcgaaagaaagattaataccggatggcataggattatcgcatgatgatcctattaaagaatttcggttatcgatggggatgcgttccattagtttgttggcggggtaacggcccaccaagacagcgatggataggggttctgagaggaaggtcccccacattggaactgagacacggtccaaactcctacgggaggcagcagtgaggaatattggtcaatggacgagagtctgaaccagccaagtagcgtgaaggatgactgccctatgggttgtaaacttcttttatacgggaataaagtggtccacgtgtggatttttgtatgtaccgtatgaataaggatcggctaactccgtgccagcagccgcggtaatacggaggatccgagcgttatccggatttattgggtttaaagggagcgtaggtggattgttaagtcagttgtgaaagtttgcggctcaaccgtaaaattgcagttgatactggcagtcttgagtacagtagaggtgggcggaattcgtggtgtagcggtgaaatgcttagatatcacgaagaactccgattgcgaaggcagctcactggactgcaactgacactgatgctcgaaagtgtgggtatcaaacaggattagataccctggtagtccacacagtaaacgatgaatactcgctgtttgcgatatacggtaagcggccaagcgaaagcgttaagtattccacctggggagtacgccggcaacggtgaaactcaaaggaattgacgggggcccgcacaagcggaggaacatgtggtttaattcgatgatacgcgaggaaccttacccgggcttaaattacatttgaataatctggaaacaggttagccgtaaggcaaatgtgaaggtgctgcatggttgtcgtcagctcgtgccgtgaggtgtcggcttaagtgccataacgagcgcaacccttatcttcagttactaacagggtcatgctgaggactctggagagactgccgtcgtaagatgtgaggaaggtggggatgacgtcaaatcagcacggcccttacgtccggggctacacacgtgttacaatggggggtacagaaggcagctacctggtgacaggatgctaatcccaaaaacctctctcagttcggatcgaagtctgcaacccgacttcgtgaagctggattcgctagtaatcgcgcatcagccatggcgcggtgaatacgttcccgggccttgtacacaccgcccgtcaagccatgaaagccgggggtacctgaagtacgtaaccgcgaggag

SEQ ID NO: 103 - DNA, 1497 bp, synthetic construct
  misc_feature 1..1497: note = Synthetic: FBI00191
  source 1..1497: mol_type = other DNA
tggagagtttgatcctggctcaggacgaacgctggcggcgtgcctaacacatgcaagtcgagcggrsmwcttcrrwkbycagcggcggacgggtgagtaacacgtgggtaacctgcctyrtagagggggatagcctyccgaaaggragattaataccgcatawmmtykcrrtatcgcatgataytkmraksaaaggagyaatctrctayragatggacccgcggcgcattagctagttggtgaggtaagggctcaccaaggcaacgatgcgtagccggcctkagagggtgaacggccacattggaactgagacacggtccagactcctacgggaggcagcagtggggaatattgcacaatgggcgaaagcctgatgcagcaacgccgcgtgagtgatgaaggccttcgggttgtaaagctctgtcttctgggacgataatgacggtaccagaggaggaagccacggctaactacgtgccagcagccgcggtaatacgtaggtggcaagcgttgtccggatttactgggygtaaaggatgcgtaggcggatatttaagtgagatgtgaaaatcccgrgcttaacttgggaactgcatttcaaactgggtatctagagtgcaggagaggaaagcggaattcctagtgtagcggtgaaatgcgtagagattaggaagaacaccagtggcgaaggcggctttctggactgtaactgacgctgaggcatgaaagcgtggggagcaaacaggattagataccctggtagtccacgccgtaaacgatgaatactaggtgtgggrrytgtcatgayytccgtgccgcagttaacacaataagtattccgcctggggagtacggtcgcaagattaaaactcaaaggaattgacgggggcccgcacaagcagcggagcatgtggtttaattcgaagcaacgcgaagaaccttacctagacttgacatctcctgaattactcgtaacggaggaagcccttcggggcaggaagacaggtggtgcatggttgtcgtcagctcgtgtcgtgagatgttgggttaagtcccgcaacgagcgcaacccttgtcgttagttgctaccattaagttgagcactctagcgagactgccgcggttaacgcggaggaaggtggggatgacgtcaaatcatcatgccccttatgtctagggctacacacgtgctacaatggtgagtacaacgagaaccgataccgcaaggtggaggaaaactcaaaaactcatcccagttcggattgcaggctgcaactcgcctgcatgaagctggagttgctagtaatcgcaaatcagaatgttgcggtgaatacgttcccgggccttgtacacaccgcccgtcacaccatgagagttgccaacacccgaagtccgtgaggtaaccgtaaggagccagcggccgaaggcgggggcagcgattggggtgaagtcgtaacaaggtagccgtaggagaacctgcggytggatcacctcctt

SEQ ID NO: 104 - DNA, 1528 bp, synthetic construct
  misc_feature 1..1528: note = Synthetic: FBI00194
  source 1..1528: mol_type = other DNA
tggagagtttgatcctggctcaggatgaacgctggcggcgtgcttaacacatgcaagtcgaacgaagcacyttdwtytgatttcttcggaatgaarawyttrgtgactgagtggcggacgggtgagtaacgcgtgggtaacctgcctcatacagggggataacagttagaaatgactgctaataccgcataagaccacagcrccgcatggtgcaggggtaaaaactccggtggtatgagatggacccgcgtctgattaggtagttggtggggtaacggcctaccaagccgacgatcagtagccgacctgagagggtgaccggccacattgggactgagacacggcccaaactcctacgggaggcagcagtggggaatattgcacaatgggggaaaccctgatgcagcgacgccgcgtgagcgatgaagtatttcggtatgtaaagctctatcagcagggaagaaaatgacggtacctgactaagaagcaccggctaaatacgtgccagcagccgcggtaatacgtatggtgcaagcgttatccggatttactgggtgtaaagggagcgtagacggagtggcaagtctgatgtgaaaacccggggctcaaccccgggactgcattggaaactgtcaatctagagtaccggagaggtaagcggaattcctagtgtagcggtgaaatgcgtagatattaggaggaacaccagtggcgaaggcggcttactggacggtaactgacgttgaggctcgaaagcgtggggagcaaacaggattagataccctggtagtccacgcygtaaacgatgactactaggtgtcgggcagcaaagctgttcggtgccgcagcaaacgcaataagtagtccacctggggagtacgttcgcaagaatgaaactcaaaggaattgacggggacccgcacaagcggtggagcatgtggtttaattcgaagcaacgcgaagaaccttacctgctcttgacatctccctgaccggcaagtaatgttgcctttccttcgggacagggatgacaggtggtgcatggttgtcgtcagctcgtgtcgtgagatgttgggttaagtcccgcaacgagcgcaacccctatctttagtagccagcggttcggccgggcactctagagagactgccagggataacctggaggaaggtggggatgacgtcaaatcatcatgccccttatgagcagggctacacacgtgctacaatggcgtaaacaaagggargcagaaccgcgaggtcgagcaaatcccaaaaataacgtctcagttcggattgtagtctgcaactcgactacatgaagctggaatcgctagtaatcgcgaatcagaatgtcgcggtgaatacgttcccgggtcttgtacacaccgcccgtcacaccatgggagtcagtaacgcccgaagtcagtgacccaaccgtaaggagggagctgccgaaggtgggaccgataactggggtgaagtcgtaacaaggtagccgtatcggaaggtgcggytggatcacctcctt

SEQ ID NO: 105 - DNA, 1367 bp, synthetic construct
  misc_feature 1..1367: note = Synthetic: FBI00197
  source 1..1367: mol_type = other DNA
cggganccntcaagcttgcttggtggtgagagtggcgaacgggtgagtaatgcgtgaccgacctgccccatgctccggaatagctcctggaaacgggtggtaatgccggatgttccacatgatcgcatgtgattgtgggaaagattctatcggcgtgggatggggtcgcgtcctatcagcttgttggtgaggtaacggctcaccaaggcttcgacgggtagccggcctgagagggcgaccggccacattgggactgagatacggcccagactcctacgggaggcagcagtggggaatattgcacaatgggcgcaagcctgatgcagcgacgccgcgtgagggatggaggccttcgggttgtaaacctcttttgtttgggagcaagccttcgggtgagtgtacctttcgaataagcgccggctaactacgtgccagcagccgcggtaatacgtagggcgcaagcgttatccggatttattgggcgtaaagggctcgtaggcggctcgtcgcgtccggtgtgaaagtccatcgcttaacggtggatctgcgccgggtacgggcgggctggagtgcggtaggggagactggaattcccggtgtaacggtggaatgtgtagatatcgggaagaacaccgatggcgaaggcaggtctctgggccgtcactgacgctgaggagcgaaagcgtggggagcgaacaggattagataccctggtagtccacgccgtaaacggtggacgctggatgtggggcacgttccacgtgttccgtgtcggagctaacgcgttaagcgtcccgcctggggagtacggccgcaaggctaaaactcaaagaaattgacgggggcccgcacaagcggcggagcatgcggattaattcgatgcaacgcgaagaaccttacctgggcttgacatgttcccgacgacgccagagatggcgtttcccttcggggcgggttcacaggtggtgcatggtcgtcgtcagctcgtgtcgtgagatgttgggttaagtcccgcaacgagcgcaaccctcgccccgtgttgccagcacgttatggtgggaactcacgggggaccgccggggttaactcggaggaaggtggggatgacgtcagatcatcatgccccttacgtccagggcttcacgcatgctacaatggccggtacagcgggatgcgacatggcgacatggagcggatccctgaaaaccggtctcagttcggatcggagcctgcaacccggctccgtgaaggcggagtcgctagtaatcgcggatcagcaacgccgcggtgaatgcgttcccgggccttgtacacaccgcccgtcaagtcatgaaagtgggcagcacccgaagccggtggcctaaccc

SEQ ID NO: 106 - DNA, 1526 bp, synthetic construct
  misc_feature 1..1526: note = Synthetic: FBI00198
  source 1..1526: mol_type = other DNA
tggagagtttgatcctggctcaggatgaacgctggcggcgtgcctaacacatgcaagtcgaacgaagcatcyyataggaagttttcggatggaatatgggatgactgagtggcggacgggtgagtaacgcgtggataacctgcctcacactgggggataacagttagaaatggctgctaataccgcataagcgcacagtrccgcatggtacggtgtgaaaaacccaggtggtgtgagatggatccgcgtctgattagccagttggcggggtaacggcccaccaaagcgacgatcagtagccgacctgagagggtgaccggccacattgggactgagacacggcccaaactcctacgggaggcagcagtggggaatattgcacaatgggcgaaagcctgatgcagcgacgccgcgtgagtgaagaagtatctcggtatgtaaagctctatcagcagggaagaaaatgacggtacctgactaagaagccccggctaactacgtgccagcagccgcggtaatacgtagggggcaagcgttatccggatttactgggtgtaaagggagcgtagacggcgatgcaagtctgaagtgaaagcccggggctcaaccccgggactgctttggaaactgtgtggctggagtgcaggagaggtaagtggaattcctagtgtagyggtgaaatgcgtagatattaggaggaacaccagtggcgaaggcggcttactggactgtaactgacgttgaggctcgaaagcgtggggagcaaacaggattagataccctggtagtccacgccgtaaacgatgaatgctaggtgtcggggggcaaagcccctcggtgccgccgctaacgcaataagcattccacctggggagtacgttcgcaagaatgaaactcaaaggaattgacggggacccgcacaagcggtggagcatgtggtttaattcgaagcaacgcgaagaaccttaccaagtcttgacatccccctgaccggacagtaacgtgtcccttccttcgggacaggggagacaggtggtgcatggttgtcgtcagctcgtgtcgtgagatgttgggttaagtcccgcaacgagcgcaacccttatccttagtagccagcayytcgggtgggcactctagggagactgccagggataacctggaggaaggtggggatgacgtcaaatcatcatgccccttatgatttgggctacacacgtgctacaatggcgtaaacaaagggargcrgccctgcgaaggtgagcaaatcccaaaaataacgtcccagttcggactgtagtctgcaacccgactacacgaagctggaatcgctagtaatcgcggatcagaatgccgcggtgaatacgttcccgggtcttgtacacaccgcccgtcacaccatgggagtcagcaacgcccgaagtcagtgacccaaccgdaaggagggagctgccgaaggcggggcaggtaactggggtgaagtcgtaacaaggtagccgtatcggaaggtgcggytggatcacctcctt

SEQ ID NO: 107 - DNA, 1396 bp, synthetic construct
  misc_feature 1..1396: note = Synthetic: FBI00199
  source 1..1396: mol_type = other DNA
tgcagtcgaacgaagcaattaaaatgaagttttcggatggatttttgattgactgagtggcggacgggtgagtaacgcgtggataacctgcctcacactgggggataacagttagaaatgactgctaataccgcataagcgcacagtaccgcagggtacggtgtgaaaaactccggtggtgtgagatggatccgcgtctgattagccagttggcggggtaacggcccaccaaagcgacgatcagtagccgacctgagagggtgaccggccacattgggactgagacacggcccaaactcctacgggaggcagcagtggggaatattgcacaatgggcgaaagcctgatgcagcgacgccgcgtgagtgaagaagtatttcggtatgtaaagctctatcagcagggaagaaaaatgacggtacctgactaagaagccccggctaactacgtgccagcagccgcggtaatacgtagggggcaagcgttatccggatttactgggtgtaaagggagcgtagacggcgaagcaagtctgaagtgaaaacccagggctcaaccctgggactgctttggaaactgttttgctagagtgtcggagaggtaagtggaattcctagtgtagcggtgaaatgcgtagatattaggaggaacaccagtggcgaaggcggcttactggacgataactgacgttgaggctcgaaagcgtggggagcaaacaggattagataccctggtagtccacgccgtaaacgatgaatgctaggtgttgggggggcaaagcccttcggtgccgtcgcaaacgcagtaagcattccacctggggagtacgttcgcaagaatgaaactcaaaggaattgacggggacccgcacaagcggtggagcatgtggtttaattcgaagcaacgcgaagaaccttaccaagtcttgacatcctcttgaccggcgtgtaacggcgccttcccttcggggcaagagagacaggtggtgcatggttgtcgtcagctcgtgtcgtgagatgttgggttaagtcccgcaacgagcgcaacccttatccttagtagccagcaggtaaagctgggcactctagggagactgccagggataacctggaggaaggtggggatgacgtcaaatcatcatgccccttatgatttgggctacacacgtgctacaatggcgtaaacaaagggaagcaagacagtgatgtggagcaaatcccaaaaataacgtcccagttcggactgtagtctgcaacccgactacacgaagctggaatcgctagtaatcgcgaatcagaatgtcgcggtgaatacgttcccgggtcttgtacacaccgcccgtcacaccatgggagtcagcaacgcccgaagtcagtgacccaactcgcaagagagg

SEQ ID NO: 108 - DNA, 1397 bp, synthetic construct
  misc_feature 1..1397: note = Synthetic: FBI00200
  source 1..1397: mol_type = other DNA
tcgnacgaagtctttaggangcttgcttccaaagagacttagtggcgaacgggtgagtaacacgtaggtaacctgcccatgtgcccgggataactgctggaaacggtagctaaaaccggataggtatgagggaggcatcttcctcatattaaagcaccttcgggtgtgaacatggatggacctgcggcgcattagctggttggtgaggtaacggcccaccaaggcgatgatgcgtagccgacctgagagggtgaacggccacattgggactgagacacggcccaaactcctacgggaggcagcagtagggaattttcgtcaatggggggaaccctgaacgagcaatgccgcgtgtgtgaagaaggtcttcggatcgtaaagcactgttgtaagtgaagaatgccacatagaggaaatgctatgtgggtgacggtagcttaccagaaagccacggctaactacgtgccagcagccgcggtaatacgtaggtggcaagcgttatccggaatcattgggcgtaaagggtgcgtaggtggcacgataagtctgaagtaaaaggcaacagctcaactgttgtatgctttggaaactgtcgagctagagtgcagaagagggcgatggaattccatgtgtagcggtaaaatgcgtagatatatggaggaacaccagtggcgaaggcggtcgcctggtctgtaactgacactgatgcacgaaagcgtggggagcaaataggattagataccctagtagtccacgccgtaaacgatgagaactaagtgttggagagattcagtgctgcagttaacgcaataagttctccgcctggggagtatgcacgcaagtgtgaaactcaaaggaattgacgggggcccgcacaagcggtggagtatgtggtttaattcgaagcaacgcgaagaaccttaccaggccttgacatggatataaatgttctagagatagaaagatagctatatatcacacaggtggtgcatggttgtcgtcagctcgtgtcgtgagatgttgggttaagtcccgcaacgagcgcaacccttgtcttctgttaccagcattaggttggggactcaggagagactgccggtgacaaaccggaggaaggtggggatgacgtcaaatcatcatgccccttatggcctgggctacacacgtactacaatggcgcctacaaagagcagcgacaccgcgaggtggagcgaatctcataaagggcgtctcagttcggattgaagtctgcaactcgacttcatgaagtcggaatcgctagtaatcgcagatcagcatgctgcggtgaatacgttctcgggccttgtacacaccgcccgtcaaaccatgggagttggtaatacccgaagccggtggcataaccgcaaggagngagc

SEQ ID NO: 109 - DNA, 1368 bp, synthetic construct
  misc_feature 1..1368: note = Synthetic: FBI00201
  source 1..1368: mol_type = other DNA
tgcagtcgaacgatgaaaccgccctcgggcggacatgaagtggcgaacgggtgagtaacacgtgaccaacctgcccccctctccgggacaaccttgggaaaccgaggctaataccggatactccctcccctgctcctgcaggggtcgggaaagcccaggcggagggggatggggtcgcggcccattaggtagtaggcggggtaacggcccacctagcccgcgatgggtagccgggttgagagaccgaccggccacattgggactgagatacggcccagactcctacgggaggcagcagtggggaattttgcgcaatgggggaaaccctgacgcagcaacgccgcgtgcgggacgacggccttcgggttgtaaaccgctttcagcagggaagaaattcgacggtacctgcagaagaagctccggctaactacgtgccagcagccgcggtaatacgtagggagcgagcgttatccggattcattgggcgtaaagagcgcgtaggcggcctctcaagcgggatctctaatccgagggctcaacccccggccggatcccgaactgggaggctcgagttcggtagaggcaggcggaattcccggtgtagcggtggaatgcgcagatatcgggaagaacaccgatggcgaaggcagcctgctgggccgcaactgacgctgaggcgcgaaagctaggggagcgaacaggattagataccctggtagtcctagccgtaaacgatggatactaggtgtggggggctccgccctccgtgccgcagccaacgcattaagtatcccgcctggggagtacggccgcaaggctaaaactcaaaggaattgacgggggcccgcacaagcagcggagcatgtggcttaattcgaagcaacgcgaagaaccttaccagggcttgacatggacgtgaagccggggaaacccggtggccgagaggagcgtccgcaggtggtgcatggctgtcgtcagctcgtgtcgtgagatgttgggttaagtcccgcaacgagcgcaacccctgccccatgttgccagcattaggttggggactcatgggggactgccggcgtcaagccggaggaaggtggggacgacgtcaagtcatcatgccctttatgccctgggctgcacacgtgctacaatggccggtacaacgggctgcgagaccgcgaggtcgagcgaatccctcaaagccggccccagttcggatcggaggctgcaacccgcctccgtgaagtcggagttgctagtaatcgcggatcagcatgccgcggtgaatacgttcccgggccttgtacacaccgcccgtcacaccacccgagtcgtctgcacccgaagccgccggccgaacccgcaaggggc

SEQ ID NO: 110 - DNA, 1367 bp, synthetic construct
  misc_feature 1..1367: note = Synthetic: FBI00205
  source 1..1367: mol_type = other DNA
attgaancttcggcngatttnnnttnnnttctagtggcggacgggtgagtaacgcgtgggtaacctgccttatacagggggataacagtcagaaatgactgctaataccgcataagcgcacagggccgcatggctcggtgtgaaaaactccggtggtataagatggacccgcgttggattagctggttggtgaggtaacggcccaccaaggcgacgatccatagccggcctgagagggtgaacggccacattgggactgagacacggcccagactcctacgggaggcagcagtggggaatattgcacaatgggggaaaccctgatgcagcgacgccgcgtgaaggaagaagtatctcggtatgtaaacttctatcagcagggaagaaaatgacggtacctgactaagaagccccggctaactacgtgccagcagccgcggtaatacgtagggggcaagcgttatccggatttactgggtgtaaagggagcgtagacggatggacaagtctgatgtgaaaggctggggctcaaccccgggactgcattggaaactgcccgtcttgagtgccggagaggtaagcggaattcctagtgtagcggtgaaatgcgtagatattaggaggaacaccagtggcgaaggcggcttactggacggtaactgacgttgaggctcgaaagcgtggggagcaaacaggattagataccctggtagtccacgcggtaaacgatgaatgctaggtgtcggggagcaaagctcttcggtgccgccgcaaacgcattaagcattccacctggggagtacgttcgcaagaatgaaactcaaaggaattgacggggacccgcacaagcggtggagcatgtggtttaattcgaagcaacgcgaagaaccttaccaagtcttgacatccctctgaccgnnnnttaaccgnntctttccttcgggacagaggagacaggtggtgcatggttgtcgtcagctcgtgtcgtgagatgttgggttaagtcccgcaacgagcgcaacccctatcctcagtagccagcggntnagccgggcactctggggagactgccagggataacctggaggaaggcggggatgacgtcaaatcatcatgccccttatgatttgggctacacacgtgctacaatggcgtaaacaaagggaagcgaacctgcgagggtgggcaaatcccaaaaataacgtcccagttcggactgtagtctgcaacccgactacacgaagctggaatcgctagtaatcgcggatcagaatgccgcggtgaatacgttcccgggtcttgtacacaccgcccgtcacaccatgggagtcagtaacgcccgaagtcagtgacctaaccgcaaggaa

SEQ ID NO: 111 - DNA, 1364 bp, synthetic construct
  misc_feature 1..1364: note = Synthetic: FBI00206
  source 1..1364: mol_type = other DNA
gcagtcgaggggcagcattttagtttgcttgcaaactaaagatggcgaccggcgcacgggtgagtaacacgtatccaacctgccgataactcggggatagcctttcgaaagaaagattaatatccgatagtatattaaaaccgcatggttttactattaaagaatttcggttatcgatggggatgcgttccattagtttgttggcggggtaacggcccaccaagactacgatggataggggttctgagaggaaggtcccccacattggaactgagacacggtccaaactcctacgggaggcagcagtgaggaatattggtcaatggacgagagtctgaaccagccaagtagcgtgaaggatgactgccctatgggttgtaaacttcttttatatgggaataaagtattccacgtgtgggattttgtatgtaccatatgaataaggatcggctaactccgtgccagcagccgcggtaatacggaggatccgagcgttatccggatttattgggtttaaagggagcgtaggtggattgttaagtcagttgtgaaagtttgcggctcaaccgtaaaattgcagttgaaactggcagtcttgagtacagtagaggtgggcggaattcgtggtgtagcggtgaaatgcttagatatcacgaagaactccgattgcgaaggcagctcactagactgcaactgacactgatgctcgaaagtgtgggtatcaaacaggattagataccctggtagtccacacagtaaacgatgaatactcgctgtttgcgatatacagtaagcggccaagcgaaagcattaagtattccacctggggagtacgccggcaacggtgaaactcaaaggaattgacgggggcccgcacaagcggaggaacatgtggtttaattcgatgatacgcgaggaaccttacccgggcttaaattgcatttgaataatctggaaacaggttagccgcaagcaaatgtgaaggtgctgcatggttgtcgtcagctcgtgccgtgaggtgtcggcttaagtgccataacgagcgcaacccttatctttagttactaacaggtcatgctgaggactctagagagactgccgtcgtaagatgtgaggaaggtggggatgacgtcaaatcagcacggcccttacgtccggggctacacacgtgttacaatggggggtacagaaggcagctacctggcgacaggatgctaatcccaaaaacctctctcagttcggatcgaagtctgcaacccgacttcgtgaagctggattcgctagtaatcgcgcatcagccatggcgcggtgaatacgttcccgggccttgtacacaccgcccgtcaagccatgaaagccggggg

SEQ ID NO: 112 - DNA, 1378 bp, synthetic construct
  misc_feature 1..1378: note = Synthetic: FBI00208
  source 1..1378: mol_type = other DNA
tcgancggagtgttttcacggaagttttcggatggaagtggttacacttagtggcggacgggtgagtaacacgtgagcaacctgcctttcagagggggataacagttggaaacgactgctaataccgcatgatattaccgggtcacatggcctggcaatcaaaggagcaatccgctgaaagatgggctcgcgtccgattagccagttggcggggtaatggcccaccaaagcgacgatcggtagccggactgagaggttgaacggccacattgggactgagacacggcccagactcctacgggaggcagcagtgggggatattgcacaatgggcgaaagcctgatgcagcgacgccgcgtgagggaagacggtcttcggattgtaaacctctgtcttaggggaagaaaatgacggtaccctaagaggaagctccggctaactacgtgccagcagccgcggtaatacgtagggagcgagcgttgtccggaattactgggtgtaaagggagcgtaggcgggatgccaagtagaatgttaaatccatcggctcaactggtggcagcgttctaaactggcgttcttgagtgaggtagaggcaggcggaattcctagtgtagcggtgaaatgcgtagatattaggaggaacaccagtggcgaaggcggcctgctgggccttaactgacgctgaggctcgaaagcgtggggagcaaacaggattagataccctggtagtccacgccgtaaacgatgattactaggtgtggggggactgaccccttccgtgccgcagttaacacaataagtaatccacctggggagtacggccgcaaggttgaaactcaaaggaattgacgggggcccgcacaagcagtggagtatgtggtttaattcgaagcaacgcgaagaaccttaccaggtcttgacatcggatgcataccatagagatatgggaagcccttcggggcatccagacaggtggtgcatggttgtcgtcagctcgtgtcgtgagatgttgggttaagtcccgcaacgagcgcaacccttattattagttgctacgcaagagcactctaatgagactgccgttgacaaaacggaggaaggtggggatgacgtcaaatcatcatgccccttatgacctgggctacacacgtactacaatggcactcaaacagagggaagcgacaccgcgaggtgaagcggatcccaaaaaagtgtctcagttcggatcgcaggctgcaacccgcctgcgtgaagtcggaattgctagtaatcgcggatcagcatgccgcggtgaatacgttcccgggccttgtacacaccgcccgtcacaccatgggagtcggtaacacccgaagccagtagcctaaccgcaagga

SEQ ID NO: 113 - DNA, 1385 bp, synthetic construct
  misc_feature 1..1385: note = Synthetic: FBI00211
  source 1..1385: mol_type = other DNA
tcgaggggcagcatggtcttancttgctaaggccgatggcgaccggcgcacgggtgagtaacacgtatccaacctgccgtctactcttggacagccttctgaaaggaagattaatacaagatggcatcatgagtccgcatgttcacatgattaaaggtattccggtagacgatggggatgcgttccattagatagtaggcggggtaacggcccacctagtcttcgatggataggggttctgagaggaaggtcccccacattggaactgagacacggtccaaactcctacgggaggcagcagtgaggaatattggtcaatgggcganagcctgaaccagccaagtagcgtgaaggatgactgccctatgggttgtaaacttcttttataaaggaataaagtcgggtatgtatacccgtttgcatgtactttatgaataaggatcggctaactccgtgccagcagccgcggtaatacggaggatccgagcgttatccggatttattgggtttaaagggagcgtagatggatgtttaagtcagttgtgaaagtttgcggctcaaccgtaaaattgcagttgatactggatatcttgagtgcagttgaggcaggcggaattcgtggtgtagcggtgaaatgcttagatatcacgaagaactccgattgcgaaggcagcctgctaagctgcaactgacattgaggctcgaaagtgtgggtatcaaacaggattagataccctggtagtccacacggtaaacgatgaatactcgctgtttgcgatatactgcaagcggccaagcgaaagcgttaagtattccacctggggagtacgccggcaacggtgaaactcaaaggaattgacgggggcccgcacaagcggaggaacatgtggtttaattcgatgatacgcgaggaaccttacccgggcttaaattgcagatgaattacggtgaaagccgtaagccgcaaggcatctgtgaaggtgctgcatggttgtcgtcagctcgtgccgtgaggtgtcggcttaagtgccataacgagcgcaacccttgttgtcagttactaacaggttccgctgaggactctgacaagactgccatcgtaagatgtgaggaaggtggggatgacgtcaaatcagcacggcccttacgtccggggctacacacgtgttacaatggggggtacagagggccgctaccacgcgagtggatgccaatccccaaaacctctctcagttcggactggagtctgcaacccgactccacgaagctggattcgctagtaatcgcgcatcagccacggcgcggtgaatacgttcccgggccttgtacacaccgcccgtcaagccatgggagccgggggtacctgaagtgcgtaaccgcgagga

SEQ ID NO: 114 - DNA, 1379 bp, synthetic construct
  misc_feature 1..1379: note = Synthetic: FBI00212
  source 1..1379: mol_type = other DNA
tgcagtcgaacgaagcgattaggaggaagttttcggatggattcctggttgactgagtggcggacgggtgagtaacgcgtggataacctgcctcacactgggggataacagttagaaatggctgctaataccgcataagcgcacagtaccgcatggtacggtgtgaaaaacccaggtggtgtgagatggatccgcgtctgattagccagttggcggggtaacggcccaccaaagcgacgatcagtagccgacctgagagggtgaccggccacgttgggactgagacagggcccaaactcctaggggaggcagcagtggggaatattgcacaatgggcgcaagcctgatgcagcgacgccgcgtgagtgaagaagtatctcggtatgtaaagctctatcagcagggaagaaaatgacggtacctgactaagaagccccggctaactacgtgccagcagccgcggtaatacgtagggggcaagcgttatccggatttactgggtgtaaagggagcgtagacggcgacgcaagtctggagtgaaagcccggggcccaaccccgggactgctttggaaactgtgttgctggagtgcaggagaggtaagtggaattcctagtgtagcggtgaaatgcgtagatattaggaggaacaccagtggcgaaggcggcttactggactgtaactgacgttgaggctcgaaagcgtggggagcaaacaggattagataccctggtagtccacgccgtaaacgatgaatgctaggtgtcggggggcaaagcccttcggtgccgccgctaacgcaataagcattccacctggggagtacgttcgcaagaatgaaactcaaaggaattgacggggacccgcacaagcggtggagcatgtggtttaattcgaagcaacgcgaagaaccttaccaagtcttgacatcctnntgaccggcnngtaacggtgcctttccttcgggacaagagagacaggtggtgcatggttgtcgtcagctcgtgtcgtgagatgttgggttaagtcccgcaacgagcgcaacccttatccttagtagccagcacgtcgtggtgggcactctagggagactgccagggataacctggaggaaggtggggatgacgtcaaatcatcatgccccttatgatttgggctacacacgtgctacaatggcgtaaacaaagggaggcgaccctgcgaaggcaagcaaatcccaaaaataacgtcccagttcggactgtagtctgcaacccgactacacgaagctggaatcgctagtaatcgcggatcagaatgccgcggtgaatacgttcccgggtcttgtacacaccgcccgtcacaccatgggagtcagcaacgcccgaagtcnagtganc

SEQ ID NO: 115 - DNA, 1417 bp, synthetic construct
  misc_feature 1..1417: note = Synthetic: FBI00220
  source 1..1417: mol_type = other DNA
tcgancgagaagagnngagaagcttgcttcttatnnattcgagtggcaaacgggtgagtaacgcgtaagcaacctgcccttcagatggggacaacagctggaaacggctgctaataccgaatacgttctttccgccgcatgacggaatgaagaaagggaggccttcgggctttcgctggaggaggggcttgcgtctgattagctggttggaggggtaacggcccaccaaggcgacgatcagtagccggtctgagaggatgaacggccacattgggactgagacacggcccagactcctacgggaggcagcantggggaatcttccgcaatggacgaaagtctgacggagcaacgccgcgtgaacgatgacggccttcgggttgtaaagttctgttatatgggacgaacagggcatcggttaatacccggtgtctttgacggtaccgtaagagaaagccacggctaactacgtgccagcagccgcggtaatannnnngtggcaagcgttgtccggaattattgggcgtaaagggcgcgcaggcggcatcgcaagtcggtcttaaaagtgcgggggcttaaccccgtgaggggaccgaaactgtgaagctcgagtgtcggagaggaaagcggaattcctagtgtagcggtgaaatgcgtagatattaggaggaacaccagtggcgaaagcggctttctggacgacaactgacgctgaggcgcgaaagccaggggagcaaacgggattagataccccggtagtcctggccgtaaacgatggatactaggtgtaggaggtatcgactccttctgtgccggagttaacgcaataagtatcccgcctgggggagtacggccgcaaggctgaaactcaaaggaattgacgggggcccgcacaagcggtggagtatgtggtttaattcgacgcaacgcgaagaaccttaccaagccttgacattgattgctacggaaagagatttccggttcttcttcggaagacaagaaaacaggtggtgcacggctgtcgtcagctcgtgtcgtgagatgttgggttaagtcccgcaacgagcgcaacccctatcttctgttgccagcacctcgggtggggactcagaagagactgccgcagacaatgcggaggaaggcggggatgacgtcaagtcatcatgccccttatggcttgggctacacacgtactacaatggctcttaatagagggaagcgaaggagcgatccggagcaaaccccaaaaacagagtcccagttcggattgcaggctgcaactcgcctgcatgaagcaggaatcgctagtaatcgcaggtcagcatactgcggtgaatacgttcccgggccttgtacacaccgcccgtcacaccacgaaagtcattcacacccgaagccggtgaggcaaccgcaagga

SEQ ID NO: 116 - DNA, 1378 bp, synthetic construct
  misc_feature 1..1378: note = Synthetic: FBI00221
  source 1..1378: mol_type = other DNA
cgaggggcagcngnnngtagcaatacttcgccggcgaccggcgcacgggtgagtaacacgtgtgcaaccaaccccgtaccgggagataacccgcggaaacgtggactaacatcccataatactggaggatcgcatgatctttcatttaaaattccggtggtacgggacgggcacgcgcgacattaggtagttggcggggtaacggcccaccaagccgacgatgtctaggggttctgagaggaaggtcccccacactggaactgagacacggtccagactcctacgggaggcagcagtgaggaatattggtcaatgggcgagagcctgaaccagccaagtcgcgtgagggaagaatggtccatggcctgtaaacctcttttgtcaaggaagaataaanngcacgtgtgcnntcttgccagtacttgacgaataagcatcggctaactccgtgccagcagccgcggtaatacgggggatgcgagcgttatccggatttattgggtttaaagggcgcgtaggcgggacgccaagtcagcggtaaaagactgcagctaaactgtagcacgccgttgaaactggcgacctcgagacgagacgagggaggcggaacaagtgaagtagcggtgaaatgcttagatatcacttggaaccccgatagcgaaggcagcttcccaggctcgatctgacgctgatgcgcgagagcgtgggtagcgaacaggattagataccctggtagtccacgccgtaaacgatgctcactggatcttggcgatacactgccagggttcaagcgaaagtattaagtgagccacctggggagtacgtcggcaacgatgaaactcaaaggaattgacgggggcccgcacaagcggaggaacatgtggtttaattcgatgatacgcgaggaaccttacccgggtttaaatgtannntgcatgtnttggaaacaggaattcccttcggggctagatacaaggtgctgcatggttgtcgtcagctcgtgccgtgaggtgtcgggttaagtcccataacgagcgcaacccctatcgccagttgccatcggttgaagccgggcactctgtcgagactgccaccgtaaggtgcgaggaaggcggggatgacgtcaaatcagcacggcccttacacccggggcgacacacgtgttacaatggccggtacagagggcagccacggggtgacccggagcgaatctctaaagccggtcgtagttcggactggagtctgcaacccgactccacgaagttggattcgctagtaatcgcgcatcagccatggcgcggtgaatacgttcccgggccttgtacacaccgcccgtcaagccatggaagccgggagtacctgaagatcgtgaccgcga

SEQ ID NO: 117 - DNA, 1391 bp, synthetic construct
  misc_feature 1..1391: note = Synthetic: FBI00224
  source 1..1391: mol_type = other DNA
tcgaacggcagcgcggggagcttgctccctggcggcgagtggcgcacgggtgagtaatacatcggaacgtgtcttctagtgggggataactgcccgaaagggcagctaataccgcatgagacctgagggtgaaagcgggggatcgcaagacctcgcgctggaagagcggccgatgtccgattagctagttggtgaggtaaaggctcaccaaggcgacgatcggtagctggtctgagaggacgaccagccacactgggactgagacacggcccagactcctacgggaggcagcagtggggaattttggacaatgggggcaaccctgatccagccatgccgcgtgcaggatgaaggtcttcggattgtaaactgcttttgtcagggacgaaaagggatgcgataacaccgcattccgctgacggtacctgaagaataagcaccggctaactacgtgccagcagccgcggtaatacgtagggtgcaagcgttaatcggaattactgggcgtaaagcgtgcgcaggcggttctgtaagatagatgtgaaatccccgggctcaacctgggaattgcatatatgactgcaggacttgagtttgtcagaggagggtggaattccacgtgtagcagtgaaatgcgtagatatgtggaagaacaccgatggcgaaggcagccctctgggacatgactgacgctcatgcacgaaagcgtgggggagcaaacaggattagataccctggtagtccacgccctaaacgatgtctactagttgttggggacgatagtccttggtaacgcagctaacgcgtgaagtagaccgcctggggagtacggtcgcaagattaaaactcaaaggaattgacggggacccgcacaagcggtggatgatgtggattaattcgatgcaacgcgaaaaaccttacctagccttgacatgccaggaagncntgagagatcaggccgtgcccgcaagggaatctggacacaggtgctgcatggctgtcgtcagctcgtgtcgtgagatgttgggttaagtcccgcaacgagcgcaacccttgtcattagttgctacgaaagggcactctaatgagactgccggtgacaaaccggaggaaggtggggatgacgtcaagtcctcatggcccttatggctagggcctcacacgtcatacaatggtcggaacagagggaagcgaagccgcgaggtgaagccaatcccagaaaaccgatcgtagtccggattgcagtctgcaactcgactgcatgaagtcggaatcgctagtaatcgcggatcagcatgccgcggtgaatacgttcccgggtcttgtacacaccgcccgtcacaccatgggagtggggttcaccagaagacgtttgcccaaccgaaaggaag

SEQ ID NO: 118 - DNA, 1398 bp, synthetic construct
  misc_feature 1..1398: note = Synthetic: FBI00226
  source 1..1398: mol_type = other DNA
tgcagtcgacganttgctctttgggaagccctcgggtggaactgtgagtatacttagtggcggacgggtgagtaacgcgtgagcaatctgccctgcaatgggggacaacagttggaaacgactgctaataccgcataagaccacgaaaccgcatggtttagaggtaaaaggattcattcgatgcaggatgagctcgcgtcccattagatagttggtgaggtaacggcccaccaagtcaacgatgggtagccgacctgagagggtgatcggccacattggaactgagaaacggtccaaactcctacgggaggcagcagtggggaatattgggcaatgggggaaaccctgacccagcaacgccgcgtgaaggaagaaggccttcgggttgtaaacttttgtcctatgggacgaaaaaaatgacggtaccataggaggaagctccggctaactacgtgccagcagccgcggtaatacgtagggagcaagcgttgtccggaattactgggcgtaaagggtgcgtaggtggccatgtaagtcaggtgtgaaagaccggggctcaaccccggggttgcacttgaaactgtgtggcttgagtacaggagagggaagtggaattcctagtgtagcggtgaaatgcgtagatattaggaggaacaccagtggcgaaggcgactttctggactgtaactgacactgaggcacgaaagcgtggggagcaaacaggattagataccctggtagtccacgccgtaaacgatggatactaggtgtggggggcgatagtcctccgtgccgaagctaacgcattaagtatcccgcctggggagtacgatcgcaaggttgaaactcaaaggaattgacgggggcccgcacaagcagcggagcatgtggtttaattcgaagcaacgcgaagaaccttaccaaggcttgacatcctctgacgcatatagagatatatgttcccttcggggcagagagacaggtggtgcatggttgtcgtcagctcgtgtcgtgagatgttgggttaagtcccgcaacgagcgcaacccttgttgctagttgccagcacgtaaaggtgggaactctagtgagactgccggggacaactcggaggaaggtggggacgacgtcaaatcatcatgccccttatgtcttgggctacacacgtgctacaatggccggtacaaagggcagcgacctcgtaagagtaagcgaatctcaaaaagccggccccagttcggattgtgggctgcaacccgcccacatgaagtcggagttgctagtaatcgcgaatcagcatgtcgcggtgaatgcgttcccgggccttgtacacaccgcccgtcacaccacggaagttgggagcacccgaagccagtggcttaaccgtaaggagagagc

SEQ ID NO: 119 - DNA, 1395 bp, synthetic construct
  misc_feature 1..1395: note = Synthetic: FBI00229
  source 1..1395: mol_type = other DNA
tgcagtnngaggggcagcgnnnnngnngctgngcttcaattgccgnnnaccggcgcacgggtgcgtaacgcgtatgcgaccgtacccataacagggggataacactgagaaattggtactaatatcccataacatcatgaggggcatcccttttggttgaaaactccggtggttatggatgggcatgcgttgtattagctggttggtgaggtaacggctcaccaaggcgacgatacatagggggactgagaggttaaccccccacattggtactgagacacggaccaaactcctacgggaggcagcagtgaggaatattggtcaatggacgcaagtctgaaccagccatgccgcgtgcaggatgacggctctatgagttgtaaactgcttttgtacgagggtaaacccggatacgtgtatccggctgaaagtatcgtacgaataaggatcggctaactccgtgccagcagccgcggtaatacggaggattcaagcgttatccggatttattgggtttaaagggtgcgtaggcggtttgataagttagaggtgaaataccggtgcttaacaccggaactgcctctaatactgttgagctagagagtagttgcggtaggcggaatgtatggtgtagcggtgaaatgcttagagatcatacagaacaccgattgcgaaggcagcttaccaaactatatctgacgttgaggcacgaaagcgtggggagcaaacaggattagataccctggtagtccacgcagtaaacgatgataactcgctgtcggcgatacacagtcggtggctaagcgaaagcgataagttatccacctggggagtacgttcgcaagaatgaaactcaaaggaattgacgggggcccgcacaagcggaggaacatgtggtttaattcgatgatacgcgaggaaccttacccgggcttgaaagttactgacgattctggaaacaggatttcccttcggggcaggaaactaggtgntgcatggttgtcgtcagctcgtgccgtgaggtgtcgggttaagtcccataacgagcgcaacccctaccgttagttgccatcaggtcaagctgggcactctggcgggactgccggtgtaagccgagaggaaggtggggatgacgtcaaatcagcacggcccttacgtccggggctacacacgtgttacaatggtaggtacagagggcagctacccagtgatgggatgcaaatctcgaaagcctatctcagttcggattggaggctgaaacccgcctccatgaagttggattcgctagtaatcgcgcatcagccatggcgcggtgaatacgttcccgggccttgtacacaccgcccgtcaagccatggaagctgggggtgcctgaagttcgngaccgcaangagc

SEQ ID NO: 120 - DNA, 1377 bp, synthetic construct
  misc_feature 1..1377: note = Synthetic: FBI00232
  source 1..1377: mol_type = other DNA
gcagtcgaggggcagcatcatcaaagcttgctttgatggatggcgaccggcgcacgggtgagtaacacgtatccaacctgccgacaacactgggatagcctttcgaaagaaagattaataccggatggcatagttttcccgcatgggataattattaaagaatttcggttgtcgatggggatgcgttccattaggcagttggcggggtaacggcccaccaaaccaacgatggataggggttctgagaggaaggtcccccacattggaactgagacacggtccaaactcctacgggaggcagcagtgaggaatattggtcaatggacgagagtctgaaccagccaagtagcgtgaaggatgactgccctatgggttgtaaacttcttttatacgggaataaagttagccacgtgtggctttttgtatgtaccgtatgaataaggatcggctaactccgtgccagcagccgcggtaatacggaggatccgagcgttatccggatttattgggtttaaagggagcgtaggcgggttgttaagtcagttgtgaaagtttgcggctcaaccgtaaaattgcagttgatactggcgaccttgagtgcaacagaggtaggcggaattcgtggtgtagcggtgaaatgcttagatatcacgaagaactccgattgcgaaggcagcttactggattgtaactgacgctgatgctcgaaagtgtgggtatcaaacaggattagataccctggtagtccacacagtaaacgatgaatactcgctgttggcgatatacagtcagcggccaagcgaaagcattaagtattccacctggggagtacgccggcaacggtgaaactcaaaggaattgacgggggcccgcacaagcggaggaacatgtggtttaattcgatgatacgcgaggaaccttacccgggcttaaattgcaactgactgaaccggaaacggttctttcttcggacagttgtgaaggtgctgcatggttgtcgtcagctcgtgccgtgaggtgtcggcttaagtgccataacgagcgcaacccttatcgatagttactagcaggtcatgctgaggactctattgagactgccgtcgtaagatgtgaggaaggtggggatgacgtcaaatcagcacggcccttacgtccggggctacacacgtgttacaatggggggtacagaaggcagctacacggcgacgtggtgctaatcccgaaagcctctctcagttcggattggagtctgcaacccgactccatgaagctggattcgctagtaatcgcgcatcagccacggcgcggtgaatacgttcccgggccttgtacacaccgcccgtcaagccatgaaagccgggggtacctnaagtacgt

SEQ ID NO: 121 - DNA, 1417 bp, synthetic construct
  misc_feature 1..1417: note = Synthetic: FBI00233
  source 1..1417: mol_type = other DNA
gtcgaacggagacactctgttcactgaatggtagaaggcggcggaaccagaaagggagcgtcggttttctactgttgagtgaatagagtgtcttagtggcggacgggtgagtaacgcgtgaataacctgccttgcagtggggaataacacaccgaaaggtgtgctaataccgcatgatgtaaggggaaggcatcttccccttaccaaagatttatcgctgtgagatggattcgcgtccgattagatagttggtgaggtaacggcccaccaagtcgacgatcggtagccggactgagaggttgaacggccacattgggactgagacacggcccagactcctacgggaggcagcagtgggggatattgcacaatgggcgcaagcctgatgcagcgacgccgcgtgtgggaagacggccctcgggttgtaaaccactggctttggggacgataatgacggtacccaaggaggaagctccggctaactacgtgccagcagccgcggtaatacgtagggagcgagcgttgtccggaattactgggtgtaaagggagcgtaggcgggagtgcaagttgaatgtttaatctatgggctcaacccatatcagcgttcaaaactgcatttcttgagtgaagtagaggttggcggaattcctagtgtagcggtgaaatgcgtagatattaggaggaacaccagtggcgaaggcggccaactgggcttttactgacgctgaggctcgaaagcgtggggagcaaacaggattagataccctggtagtccacgccgtaaacgatgaatactaggtgtggggggactgaccccttccgtgccgcagttaacacaataagtattccacctggggagtacggccgcaaggctgaaactcaaaggaattgacgggggcccgcacaagcagtggattatgtggtttaattcgaagcaacgcgaagaaccttaccaggccttgacatctcctgagtagcctagagataggtgatgcccttcggggcaggaagacaggtggtgcatggttgtcgtcagctcgtgtcgtgagatgttgggttaagtcccgcaacgagcgcaacccttacggatagttgctacgcaagagcactctatcaggactgccgttgacaaaacggaggaaggtggggatgacgtcaaatcatcatgccccttatggcctgggctacacacgtaatacaatggcgtttaacagagggaagcaagaccgcgaggtggagcgaatcctcaaaaggcgtctcagttcagattgcaggctgcaacccgcctgcatgaagtcggaattgctagtaatcgcggatcagcatgccgcggtgaatacgttctcgggccttgtacacaccgcccgtcacaccatggaagtcggtaacacccgaagtcagtagcctaaccgcaa

SEQ ID NO: 122 - DNA, 1387 bp, synthetic construct
  misc_feature 1..1387: note = Synthetic: FBI00235
  source 1..1387: mol_type = other DNA
tgcngtcgaggggcagcacggtgtagcaatacactggtggcgaccggcgcacgggtgcgtaacgcgtatgcaacctacccataacagggggataacactgagaaattggtactaataccccataacatcaggaccggcatcggttctggttgaaaactccggtggttatggatgggcatgcgttgtattagctggttggtgaggtaacggctcaccaaggcaacgatacatagggggactgagaggttaaccccccacattggtactgagacacggaccaaactcctacgggaggcagcagtgaggaatattggtcaatggacgcaagtctgaaccagccatgccgcgtgcaggaagacggctctatgagttgtaaactgcttttgtacgagagtaaacgctcttacgtgtaagagcctgaaagtatcgtacgaataaggatcggctaactccgtgccagcagccgcggtaatacggaggatccaagcgttatccggatttattgggtttaaagggtgcgtaggcggtttgataagttagaggtgaaataccggtgcttaacaccgggactgcctctaatactgttgaactagagagtagttgcggtaggcggaatgtatggtgtagcggtgaaatgcttagagatcatacagaacaccgattgcgaaggcagcttaccaaactatatctgacgttgaggcacgaaagcgtggggagcaaacaggattagataccctggtagtccacgcagtaaacgatgataactcgctgtcggcgatacacagtcggcggctaagcgaaagcgataagttatccacctggggagtacgttcgcaagaatgaaactcaaaggaattgacgggggcccgcacaagcggaggaacatgtggtttaattcgatgatacgcgaggaaccttacccgggcttgaaagttactgacgattctggaaacaggatttcccttcggggcaggaaactaggtgctgcatggttgtcgtcagctcgtgccgtgaggtgtcgggttaagtcccataacgagcgcaacccctaccgttagttgccatcaggtcaagctgggcactctggcgggactgccggtgtaagccgagaggaaggtggggatgacgtcaaatcagcacggcccttacgtccggggctacacacgtgttacaatggtaggtacagagggcagctacccagtgatgggatgcgaatctcgaaagcctatctcagttcggatcggaggctgaaacccgcctccgtgaagttggattcgctagtaatcgcgcatcagccatggcgcggtgaatacgttcccgggccttgtacacaccgcccgtcaagccatggaagctgggggtgcctgaagttcgtgaccgcaaggag

SEQ ID NO: 123 - DNA, 1527 bp, synthetic construct
  misc_feature 1..1527: note = Synthetic: FBI00236
  source 1..1527: mol_type = other DNA
tggagagtttgatcctggctcaggatgaacgctggcggcgtgcctaacacatgcaagtcgaacgaagttacacagaggaagttttcggatggaatcrgtataacttagtggcggacgggtgagtaacgcgtgggaaacctgccctgtaccgggggataacacttagaaataggtgctaataccgcataagcgcacrgmwyyrcatgrwkchgtgtgaaaaactccggtggtacaggatggtcccgcgtctgattagccagttggcagggtaacggcctaccaaagcgacgatcagtagccggcctgagagggtgaacggccacattgggactgagacacggcccaaactcctacgggaggcagcagtggggaatattgcacaatgggggaaaccctgatgcagcgacgccgcgtgagtgaagaagtatttcggtatgtaaagctctatcagcagggaagaaaatgacggtacctgactaagaagccccggctaactacgtgccagcagccgcggtaatacgtagggggcaagcgttatccggatttactgggtgtaaagggagcgtagacggcatggcaagccagatgtgaaaacccagggctcaaccttgggattgcatttggaactgccaggctggagtgcaggagaggtaagcggaattcctagtgtagcggtgaaatgcgtagatattaggaggaacaccagtggcgaaggcggcttactggactgtaactgacgttgaggctcgaaagcgtggggagcaaacaggattagataccctggtagtccacgcggtaaacgatgattgctaggtgtaggtgggtatggacccatcggtgccgcagctaacgcaataagcaatccacctggggagtacgttcgcaagaatgaaactcaaaggaattgacggggacccgcacaagcggtggagcatgtggtttaattcgaagcaacgcgaagaaccttaccaagtcttgacatcccawtgacgyrcctgtaaagaggyrttcccttcggggcawtggagacaggtggtgcatggttgtcgtcagctcgtgtcgtgagatgttgggttaagtcccgcaacgagcgcaacccttattcttagtagccagcaggtaragctgggcactctaaggagactgccggggataacccggaggaaggcggggatgacgtcaaatcatcatgccccttatgatttgggctacacacgtgctacaatggcgtaaacaaagggaagcgagacagtgatgtggagcaaatcccagaaataacgtctcagttcggattgtagtctgcaactcgactacatgaagctggaatcgctagtaatcgcgaatcagcatgtcgcggtgaatacgttcccgggtcttgtacacaccgcccgtcacaccatgggagttggaaatgcccgaagtctgtgacctaaccgaaagggaggagcagccgaaggcaggtctgataactggggtgaagtcgtaacaaggtagccgtatcggaaggtgcggytggatcacctcctt

SEQ ID NO: 124 - DNA, 1529 bp, synthetic construct
  misc_feature 1..1529: note = Synthetic: FBI00237
  source 1..1529: mol_type = other DNA
tggagagtttgatcctggctcaggatgaacgctggcggcatgcctaatacatgcaagtcgaacgaagtgaagatagcttgctattggaacttagtggcgaacgggtgagtaacacgtagataacctgcctgtatgaccgggataacagttggaaacgactgctaataccggataggcagagaggaggcatctcttctctgttaaagttgggacacaacgcaaacagatggatctgcggtgcattagctagttggtgaggtaacggcccaccaaggcgatgatgcatagccggcctgagagggcgaacggccacattgggactgagacacggcccaaactcctacgggaggcagcagtagggaattttcggcaatgggggaaaccctgaccgagcaatgccgcgtgagtgaagacggccttcgggttgtaaagctctgttgtaagggaagaacggcatagagagggaatgctctatgagtgacggtaccttaccagaaagccacggctaactacgtgccagcagccgcggtaatacgtaggtggcaagcgttatccggaattattgggcgtaaagggtgcgtaggcggcgagataagtctgaggtaaaagcccgtggctcaaccacggtaagccttggaaactgtctggctggagtgcaggagaggacaatggaattccatgtgtagcggtaaaatgcgtagatatatggaggaacaccagtggcgaaggcggttgtctggcctgtaactgacgctgaagcacgaaagcgtggggagcaaataggattagataccctagtagtccacgccgtaaacgatgagaactaagtgttggggaaactcagtgctgcagttaacgcaataagttctccgcctggggagtatgcacgcaagtgtgaaactcaaaggaattgacgggggcccgcacaagcggtggagtatgtggtttaattcgacgcaacgcgaagaaccttaccaggccttgacatggtatcaaaggccctagagatagggagatagttatggtacacacaggtggtgcatggttgtcgtcagctcgtgtcgtgagatgttgggttaagtcccgcaacgagcgcaacccttgtttctagttaccaacagtaagatggggactctagagagactgccggtgacaaaccggaggaaggtggggatgacgtcaaatcatcatgccccttatggcctgggctacacacgtactacaatggcgtctacaaagagcagcgagcaggtgactgtaagcgaatctcataaaggacgtctcagttcggattgaagtctgcaactcgacttcatgaagtcggaatcgctagtaatcgcggatcagcatgccgcggtgaatacgttctcgggccttgtacacaccgcccgtcaaaccatgggagttgataatacccgaagccggtggcctaaccatttatggagggagccgtcgaaggtaggatcgatgactggggttaagtcgtaacaaggtatccctacgggaacgtgcggytggatcacctcctt

SEQ ID NO: 125 - DNA, 1395 bp, synthetic construct
  misc_feature 1..1395: note = Synthetic: FBI00238
  source 1..1395: mol_type = other DNA
anncntgcagtcgaggggcagcatgaagtagcaatactttgatggcgaccggcggacgggtgcgtaacgcgtatgcaacctaccttaaacagggggataacactgagaaattggtactaataccccatattattcgagacggcatcgtcttgggttgaaaactccggtggtttaagatgggcatgcgttgtattagctggttggtgaggtaacggctcaccaaggcgacgatacatagggggactgagaggtcaaccccccacattggtactgagacacggaccaaactcctacgggaggcagcagtgaggaatattggtcaatggacgcaagtctgaaccagccatgccgcgtgcaggaagacggctctatgagttgtaaactgcttttgtacgagggtaacagcatctacgcgtaggtgcatgaaagtatcgtacgaataaggatcggctaactccgtgccagcagccgcggtaatacggaggatccaagcgttatccggatttattgggtttaaagggtgcgtaggcggttttataagttagaggttaaatatcggagcttaactccgttatgcctctaatactgtaggactagagaatagttgcggtaggcggaatgtatggtgtagcggtgaaatgcttagagatcatacagaacaccgattgcgaaggcagcttaccaaactatgactgacgttgaggcacgaaagcgtggggagcaaacaggattagataccctggtagtccacgcagtaaacgatgataactcgttgtcggcgatacacagtcggtgactaagcgaaagcgataagttatccacctggggagtacgttcgcaagaatgaaactcaaaggaattgacgggggcccgcacaagcggaggaacatgtggtttaattcgatgatacgcgaggaaccttacccgggcttgaaagttatcgacggacctagaaataggtcttccctacggggcgagaaactaggtgctgcatggttgtcgtcagctcgtgccgtgaggtgtcgggttaagtcccataacgagcgcaacccctaccgttagttgccatcaggtcaagctgggcactctggcgggactgccggtgtaagccgagaggaaggtggggatgacgtcaaatcagcacggcccttacgtccggggccacacacgtgttacaatggtaggtacagagggcagctaccctgcgaagggatgcgaatctcgaaagcctatctcagttcggattggaggctgaaacccgcctccatgaagttggattcgctagtaatcgcgcatcagccatggcgcggtgaatacgttcccgggccttgtacacaccgcccgtcaagccatggaagctgggggtgcctgaagttcgtgaccgcaaggagcgac

SEQ ID NO: 126 - DNA, 1363 bp, synthetic construct
  misc_feature 1..1363: note = Synthetic: FBI00243
  source 1..1363: mol_type = other DNA
tgcagtcgancggtgaagaggagcttgctcctcggancagtggcggacgggtgagtaacacgtgagcaacctggctctaagagggggacaacagttggaaacgactgctaataccgcataacgtatcgggatggcatcttcctgataccaaagattttatcgcttagagatgggctcgcgtctgattagatagttggcggggtaacggcccaccaagtcgacgatcagtagccggactgagaggttgaacggccacattgggactgagacacggcccagactcctacgggaggcagcagtgggggatattggacaatgggggcaaccctgatccagcgacgccgcgtgagggaagaaggttttcggattgtaaacctctgttgacggagaaaaaaatgatggtatccgtttagaaagccacggctaactacgtgccagcagccgcggtaatacgtaggtggcaagcgttgtccggaattactgggtgtaaagggagtgtaggcgggatatcaagtcagaagtgaaaattacgggctcaactcgtaacctgcttttgaaactgacattcttgagtgaagtagaggcaagcggaattcctagtgtagcggtgaaatgcgtagatattaggaggaacaccagtggcgaaggcggcttgctgggcttttactgacgctgaggctcgaaagcgtggggagcaaacaggattagataccctggtagtccacgccgtaaacgatgattactaggtgtggggggattgaccccttccgtgccggagtaaacacaataagtaatccacctggggagtacgaccgcaaggttgaaactcaaaggaattgacgggggcccgcacaagcagtggagtatgtggtttaattcgacgcaacgcgaagaaccttaccaggtcttgacatcgagtgaccgcctaagagattaggctttcccttcggggacacaaagacaggtggtgcatggttgtcgtcagctcgtgtcgtgagatgttgggttaagtcccgcaacgagcgcaacccttatcattagttgctacgcaagagcactctaatgagactgccgttgacaaaacggaggaaggtggggatgacgtcaaatcatcatgccctttatgacctgggctacacacgtactacaatggcgtttaacaaagagaagcaaagccgcgaggcagagcaaatctccaaaaaacgtctcagttcggattgtaggctgcaactcgcctacatgaagtcggaattgctagtaatcgtaggtcagcatactacggtgaatacgttcccgggccttgtacacaccgcccgtcaaaccatgagagttggcaacacccgaagtcggtagtctaancgca

SEQ ID NO: 127 - DNA, 1371 bp, synthetic construct
  misc_feature 1..1371: note = Synthetic: FBI00244
  source 1..1371: mol_type = other DNA
tcgaacgagcgagagagagcttgctttctcgagcgagtggcgaacgggtgagtaacgcgtgaggaacctgcctcaaagagggggacaacagttggaaacgactgctaataccgcataagcccacggcccggcatcgggcagagggaaaaggagcaatccgctttgagatggcctcgcgtccgattagctagttggtgaggtaacggcccaccaaggcgacgatcggtagccggactgagaggttgaacggccacattgggactgagacacggcccagactcctacgggaggcagcagtggggaatattgcacaatgggggaaaccctgatgcagcgacgccgcgtggaggaagaaggtcttcggattgtaaactcctgttgttgaggaagataatgacggtactcaacaaggaagtgacggctaactacgtgccagcagccgcggtaaaacgtaggtcacaagcgttgtccggaattactgggtgtaaagggagcgcaggcgggaagacaagttggaagtgaaatccatgggctcaacccatgaactgctttcaaaactgtttttcttgagtagtgcagaggtaggcggaattcccggtgtagcggtggaatgcgtagatatcgggaggaacaccagtggcgaaggcggcctactgggcaccaactgacgctgaggctcgaaagtgtgggtagcaaacaggattagataccctggtagtccacactgtaaacgatgattactaggtgttggaggattgaccccttcagtgccgcagttaacacaataagtaatccacctggggagtacgaccgcaaggttgaaactcaaaggaattgacgggggcccgcacaagcagtggagtatgtggtttaattcgacgcaacgcgaagaaccttaccaagtcttgacatcctgcgacggtgctggaaacagtactttccttcgggacgcagagacaggtggtgcatggttgtcgtcagctcgtgtcgtgagatgttgggttaagtcccgcaacgagcgcaacccttatggtcagttactacgcaagaggactctggccagactgccgttgacaaaacggaggaaggtggggatgacgtcaaatcatcatgccctttatgacttgggctacacacgtactacaatggcgttaaacaaagagaagcaagaccgcgaggtggagcaaaactcagaaacaacgtcccagttcggactgcaggctgcaactcgcctgcacgaagtcggaattgctagtaatcgcagatcagcatgctgcggtgaatacgttcccgggccttgtacacaccgcccgtcacaccatgagagccggggggacccgaagtcggtagtctaaccgcaaggaggacncc

SEQ ID NO: 128 - DNA, 1410 bp, synthetic construct
  misc_feature 1..1410: note = Synthetic: FBI00245
  source 1..1410: mol_type = other DNA
agtcgaacggagaacttatttcggtaagttcttagtggcgaacgggtgagtaacgcgtgggcaacctgccctccagttggggacaacattccgaaagggatgctaataccgaatgtcctccctcctccgcatggaggagggaggaaagatggcctctgcttgcaagctatcgctggaagatgggcccgcgtctgattagctagttggtggggtaacggctcaccaaggcgatgatcagtagccggtctgagaggatgaacggccacattgggactgagacacggcccaaactcctacgggaggcagcagtggggaatcttccgcaatggacgaaagtctgacggagcaacgccgcgtgagtgatgaaggtcttcggattgtaaaactctgttgttagggacgaaagcaccgtgttcgaacaggtcatggtgttgacggtacctaacgaggaagccacggctaactacgtgccagcagccgcggtaatacgtaggtggcaagcgttgtccggaattattgggcgtaaagagcatgtaggcgggcttttaagtctgacgtgaaaatgcggggcttaaccccgtatggcgttggatactggaagtcttgagtgcaggagaggaaaggggaattcccagtgtagcggtgaaatgcgtagatattgggaggaacaccagtggcgaaggcgcctttctggactgtgtctgacgctgagatgcgaaagccagggtagcaaacgggattagataccccggtagtcctggccgtaaacgatggatactaggtgtaggaggtatcgaccccttctgtgccggagttaacgcaataagtatcccgcctggggactacgatcgcaagattgaaactcaaaggaattgacgggggcccgcacaagcggtggagtatgtggtttaattcgacgcaacgcgaagaaccttaccaaggcttgacattgagtgaaagacctagagataggtccctcccttcggggacacgaaaacaggtggtgcatggctgtcgtcagctcgtgtcgtgagatgttgggttaagtcccgcaacgagcgcaacccctatcctatgttaccagcgcgtaaaggcggggactcataggagactgccagggataacttggaggaaggcggggatgacgtcaagtcatcatgccccttatgtcttgggctacacacgtactacaatggtcggcaacaaagggcagcgaaaccgcgaggtggagcaaatcccagaaacccgaccccagttcggatcgtaggctgcaacccgcctacgtgaagttggaatcgctagtaatcgcaggtcagcatactgcggtgaatacgttcccgggccttgtacacaccgcccgtcacaccacgaaagttggtaacacccgaagccggtgagataaccttttag

SEQ ID NO: 129 - DNA, 1377 bp, synthetic construct
  misc_feature 1..1377: note = Synthetic: FBI00248
  source 1..1377: mol_type = other DNA
tcnagcgagaagccattgactganacttcggtnagaaggatatggtggaaagcggcggacgggtgagtaacgcgtaggcaacctgccccttacagagggatagccattggaaacgatgattaaaacctcataacgcatcccccccacatggaggggaagccaaagattcatcggtaagggatgggcctgcgtctgattagcttgttggcggggtaacggcccaccaaggcgacgatcagtagccgacctgagagggtgatcggccacattggaactgagacacggtccaaactcctacgggaggcagcagtggggaatattgcacaatgggcgaaagcctgatgcagcaacgccgcgtgagggatgaaggccttcgggtcgtaaacctctgtccttggggaagaaacaaatgacggtacccatggaggaagccccggctaactacgtgccagcagccgcggtaatacgtagggggcgagcgttatccggaattattgggcgtaaagagtgcgtaggtggttacctaagcgcagggtctaaggcaatggctcaaccattgttcgccctgcgaactgggctacttgagtgcaggagaggaaagcggaattcctagtgtagcggtgaaatgcgtagatattaggaggaacaccagtggcgaaggcggctttctggactgttactgacactgaggcacgaaagcgtggggagcaaacaggattagataccctggtagtccacgccgtaaacgatgagcactaggtgtcggggccgcaaggcttcggtgccgcagttaacgcattaagtgctccgcctggggagtacgcacgcaagtgtgaaactcaaaggaattgacggggacccgcacaagcagcggagcatgtggtttaattcgaagcaacgcgaagaaccttaccaggacttgacatcccactgacagatccctaaccggatccttcttcggacagtggagacaggtggtgcatggttgtcgtcagctcgtgtcgtgagatgttgggttaagtcccgcaacgagcgcaacccttgccattagttgccatcattcagttgggcactctaatgggactgccggggacaactcggaggaaggtggggatgacgtcaaatcatcatgccccttatgttctgggctacacacgtgctacaatggccggtacagcaagaagcaaaaccgcgaggtggagcaaatctcaaaaaccggtcccagttcggactgcaggctgaaacccgcctgcacgaagccggagttgctagtaatcgtggatcagaatgccacggtgaatgcgttcccgggtcttgtacacaccgcccgtcacaccatggaagttgggggtgcccgaagccggcagggaaatatgc

SEQ ID NO: 130 - DNA, 1378 bp, synthetic construct
  misc_feature 1..1378: note = Synthetic: FBI00251
  source 1..1378: mol_type = other DNA
gtcgnncgggatccatcaggctttgcttggtggtgagagtggcgaacgggtgagtaatgcgtgaccgacctgccccatacaccggaatagctcctggaaacgggtggtaatgccggatgctccgactcctcgcatggggtgtcgggaaagatttcatcggtatgggatggggtcgcgtcctatcaggtagtcggcggggtaacggcccaccgagcctacgacgggtagccggcctgagagggcgaccggccacattgggactgagatacggcccagactcctacgggaggcagcagtggggaatattgcacaatgggcgcaagcctgatgcagcgacgccgcgtgcgggatgacggccttcgggttgtaaaccgcttttgatcgggagcaagccttcgggtgagtgtacctttcgaataagcaccggctaactacgtgccagcagccgcggtaatacgtagggtgcaagcgttatccggaattattgggcgtaaagggctcgtaggcggttcgtcgcgtccggtgtgaaagtccatcgcttaacggtggatctgcgccgggtacgggcgggctggagtgcggtaggggagactggaattcccggtgtaacggtggaatgtgtagatatcgggaagaacaccaatggcgaaggcaggtctctgggccgttactgacgctgaggagcgaaagcgtggggagcgaacaggattagataccctggtagtccacgccgtaaacggtggatgctggatgtggggcccgttccacgggttccgtgtcggagctaacgcgttaagcatcccgcctggggagtacggccgcaaggctaaaactcaaagaaattgacgggggcccgcacaagcggcggagcatgcggattaattcgatgcaacgcgaagaaccttacctgggcttgacatgttcccgacagccgtagagatatggcctcccttcggggcgggttcacaggtggtgcatggtcgtcgtcagctcgtgtcgtgagatgttgggttaagtcccgcaacgagcgcaaccctcgccctgtgttgccagcacgtcatggtgggaactcacgggggaccgccggggtcaactcggaggaaggtggggatgacgtcagatcatcatgccccttacgtccagggcttcacgcatgctacaatggccggtacaacgggatgcgacacggcgacgtggagcggatccctgaaaaccggtctcagttcggattggagtctgcaacccgactccatgaaggcggagtcgctagtaatcgcggatcagcaacgccgcggtgaatgcgttcccgggccttgtacacaccgcccgtcaagtcatgaaagtgggtagcacccgaagccggtggcctaaccctttg

SEQ ID NO: 131 - DNA, 1202 bp, synthetic construct
  misc_feature 1..1202: note = Synthetic: FBI00254
  source 1..1202: mol_type = other DNA
gattcttcggatganngnnnggtgactgagtggcggacgggtgagtaacgcgtgggtaacctgccctgtacagggggataacagctggaaacggctgctaataccgcataagcgcacgaggagacatctccttgtgtgaaaaactccggtggtacaggatgggcccgcgtctgattagctggttggcagggtaacggcctaccaaggcaacgatcagtagccggtctgagaggatgaacggccacattggaactgagacacggtccaaactcctacgggaggcagcagtggggaatattgcacaatgggggaaaccctgatgcagcaacgccgcgtgagtgaagaagtatttcggtatgtaaagctctatcagcagggaagataatgacggtacctgactaagaagctccggctaaatacgtgccagcagccgcggtaatacgtatggagcaagcgttatccggatttactgggtgtaaagggtgcgtaggtggcagtgcaagtcagatgtgaaaggccggggctcaaccccggagctgcatttgaaactgctcgggctagagtacaggagaggcaggcggaattcctagtgtagcggtgaaatgcgtagatattaggaggaacaccagtggcgaaggcggcctgctggactgttactgacactgaggcacgaaagcgtggggagcaaacaggattagataccctggtagtccacgccgtaaacgatgaatactaggtgtcggggccgtataggcttcgggtgccgccgctaacgcagtaagtattccacctggggagtacgttcgcaagaatgaaactcaaaggaattgacggggacccgcacaagcggtggagcatgtggtttaattcgaagcaacgcgaaagaaccttaccaggtcttgacatccttctgaccactccgtaatggagttttccttcgggacagaagagacaggtggnntatggttgtcgtcagctcgtgtcgtgagatgttgggttaagtcccgcaacgagcgcaacccctatcttcagtagccagcaggtaaggctgggcactctggagagactgccagggataacctggaggaaggtggggacgacgtcaaatcatcatgccccttatgatctgggcgacacacgtgctacaatggcggtcacagagtgaggcgaacccgggagggggagcaaaccacaacaaggcc

SEQ ID NO: 132 - DNA, 1391 bp, synthetic construct
  misc_feature 1..1391: note = Synthetic: FBI00255
  source 1..1391: mol_type = other DNA
tgcagtcgagcgnngcggtttcnatgaagttttcggatggatttgaaattgacttagcggcggacgggtgagtaacgcgtgggtaacctgccttacactgggggataacagttagaaatgactgctaataccgcataagcgcacagggccgcatggtccggtgtgaaaaactccggtggtgtaagatggacccgcgtctgattaggtagttggcggggtaacggcccaccaagccgacgatcagtagccgacctgagagggtgaccggccacattgggactgagacacggcccaaactcctacgggaggcagcagtggggaatattggacaatgggcgaaagcctgatccagcgacgccgcgtgagtgaagaagtatttcggtatgtaaagctctatcagcagggaagaaaatgacggtacctgactaagaagccccggctaactacgtgccagcagccgcggtaatacgtagggggcaagcgttatccggatttactgggtgtaaagggagcgtagacggtttagcaagtctgaagtgaaagcccggggctcaaccccggtactgctttggaaactgttagacttgagtgcaggagaggtaagtggaattcctagtgtagcggtgaaatgcgtagatattaggaggaacaccagtggcgaaggcggcttactggactgtaactgacgttgaggctcgaaagcgtggggagcaaacaggattagataccctggtagtccacgccgtaaacgatgaatactaggtgtcggggggcaaagcccttcggtgccgccgcaaacgcaataagtattccacctggggagtacgttcgcaagaatgaaactcaaaggaattgacggggacccgcacaagcggtggagcatgtggtttaattcgaagcaacgcgaagaaccttaccaagtcttgacatcccattgaaannnnnttaaccggtgtccctcttcggagcaatggagacaggtggtgcatggttgtcgtcagctcgtgtcgtgagatgttgggttaagtcccgcaacgagcgcaacccttatccttagtagccagcgagtagagtcgggcactctggggagactgccagggataacctggaggaaggtggggatgacgtcaaatcatcatgccccttatgatttgggctacacacgtgctacaatggcgtaaacaaagggaagcaaaggagcgatccggagcaaaccccaaaaataacgtctcagttcggattgcaggctgcaactcgcctgcatgaagctggaatcgctagtaatcgcgaatcagaatgtcgcggtgaatacgttcccgggtcttgtacacaccgcccgtcacaccatgggagttggtaacgcccgaagtcagtgacccaaccgcaagnag

SEQ ID NO: 133 - DNA, 1374 bp, synthetic construct
  misc_feature 1..1374: note = Synthetic: FBI00258
  source 1..1374: mol_type = other DNA
tcgagcgaaccacttcggtggtgagcggcgaacgggtgagtaacacgtaggttatctgcccatcagacggggacaacgattggaaacgatcgctaataccggataggacgaaagtttaaaggtgcttcggcaccactgatggatgagcctgcggcgcattagctagttggtagggtaaaggcctaccaaggcgacgatgcgtagccgacctgagagggtgaacggccacactgggactgagacacggcccagactcctacgggaggcagcagtagggaatcttcggcaatgggcgaaagcctgaccgagcaacgccgcgtgaatgatgaaggccttcgggttgtaaaattctgttataagggaagaatggctctagtaggaaatggctagagtgtgacggtaccttatgagaaagccacggctaactacgtgccagcagccgcggtaatacgtaggtggcgagcgttatccggaattattgggcgtaaagagcgcgcaggtggttgattaagtctgatgtgaaagcccacggcttaacccgtggagggtcattggaaactggtcaacttgagtgcagaagagggaagtggaattccatgtgtagcggtgaaatgcgtagagatatggaggaacaccagtggcgaaggcggcttcctggtctgtaactgacactgaggcgcgaaagcgtggggagcaaacaggattagataccctggtagtccacgccgtaaacgatgagtgctaagtgttgggggtcgaacctcagtgctgaagttaacgcattaagcactccgcctggggagtacggtcgcaagactgaaactcaaaggaattgacggggacccgcacaagcggtggagcatgtggtttaattcgaagcaacgcgaagaaccttaccaggtcttgacataccagtgaccgtcctagagataggattttcccttcggggacaatggatacaggtggtgcatggttgtcgtcagctcgtgtcgtgagatgttgggttaagtcccgcaacgagcgcaacccctgtcgttagttgccagcattcagttggggactctaacgagactgccagtgacaaactggaggaaggtggggatgacgtcaaatcatcatgccccttatgacctgggctacacacgtgctacaatggttggtacaaagagaagcgaagcggtgacgtggagcaaacctcataaagccaatctcagttcggattgtaggctgcaactcgcctacatgaagttggaatcgctagtaatcgcgaatcagcatgtcgcggtgaatacgttcccgggtcttgtacacaccgcccgtcacaccacgagagtttacaacacccgaagtcagtggcctaaccgcaaggaggga

SEQ ID NO: 134 - DNA, 1389 bp, synthetic construct
  misc_feature 1..1389: note = Synthetic: FBI00260
  source 1..1389: mol_type = other DNA
tgcagtcgnacgaagcactttatttgatttccttcgggantgattattttgtgactgagtggcggacgggtgagtaacgcgtgggtaacctgccttgtacagggggataacagttggaaacggctgctaataccgcataagcgcacagcatcgcatgatgcagtgtgaaaaactccggtggtataagatggacccgcgttggattagctagttggtgaggtaacggcccaccaaggcgacgatccatagccgacctgagagggtgaccggccacattgggactgagacacggcccaaactcctacgggaggcagcagtggggaatattgcacaatgggcgaaagcctgatgcagcgacgccgcgtgagcgaagaagtatttcggtatgtaaagctctatcagcagggaagataatgacggtacctgactaagaagcaccggctaaatacgtgccagcagccgcggtaatacgtatggtgcaagcgttatccggatttactgggtgtaaagggagcgcaggcggtgcggcaagtctgatgtgaaagcccggggctcaaccccggtactgcattggaaactgtcgtactagagtgtcggaggggtaagcggaattcctagtgtagcggtgaaatgcgtagatattaggaggaacaccagtggcgaaggcggcttactggacgataactgacgctgaggctcgaaagcgtggggagcaaacaggattagataccctggtagtccacgccgtaaacgatgaatactaggtgttgggaagcattgcttctcggtgccgtcgcaaacgcagtaagtattccacctggggagtacgttcgcaagaatgaaactcaaaggaattgacggggacccgcacaagcggtggagcatgtggtttaattcgaagcaacgcgaagaaccttaccaagtcttgacatccttctgaccggtacttaaccgtaccttctcttcggagcaggagtgacaggtggtgcatggttgtcgtcagctcgtgtcgtgagatgttgggttaagtcccgcaacgagcgcaacccttatctttagtagccagcggtttggccgggcactctagagagactgccagggataacctggaggaaggcggggatgacgtcaaatcatcatgccccttatgacttgggctacacacgtgctacaatggcgtaaacaaagggaagcaaagctgtgaagccgagcaaatctcaaaaataacgtctcagttcggactgtagtctgcaacccgactacacgaagctggaatcgctagtaatcgcagatcagaatgctgcggtgaatacgttcccgggtcttgtacacaccgcccgtcacaccatgggagttgggaatgcccgaagccagtgacctaaccgaaag

SEQ ID NO: 135 - DNA, 1524 bp, synthetic construct
  misc_feature 1..1524: note = Synthetic: FBI00263
  source 1..1524: mol_type = other DNA
tggagagtttgatcctggctcaggatgaacgctagctacaggcttaacacatgcaagtcgaggggcatcagtttggtttgcttgcaaaccaaagctggcgaccggcgcacgggtgagtaacacgtatccaacctgcctcatactcggggatagcctttcgaaagaaagattaatatccgatagcatatatttcccgcatgggttttatattaaagaaattcggtatgagatggggatgcgttccattagtttgttgggggggtaacggcccaccaagactacgatggataggggttctgagaggaaggtcccccacattggaactgagacacggtccaaactcctacgggaggcagcagtgaggaatattggtcaatggacgcgagtctgaaccagccaagtagcgtgaaggatgactgccctatgggttgtaaacttcttttatatgggaataaagttgtccacgtgtggatttttgtatgtaccatatgaataaggatcggctaactccgtgccagcagccgcggtaatacggaggatccgagcgttatccggatttattgggtttaaagggagcgtaggcggattgttaagtcagttgtgaaagtttgcggctcaaccgtaaaattgcagttgatactggcagtcttgagtgcagtagaggtgggcggaattcgtggtgtagcggtgaaatgcttagatatcacgaagaactccgattgcgaaggcagctcactggagtgtaactgacgctgatgctcgaaagtgtgggtatcaaacaggattagataccctggtagtccacacagtaaacgatgaatactcgctgtttgcgatatacagtaagcggccaagcgaaagcattaagtattccacctggggagtacgccggcaacggtgaaactcaaaggaattgacgggggcccgcacaagcggaggaacatgtggtttaattcgatgatacgcgaggaaccttacccgggcttaaattgcaaatgaattatggggaaacccatacgccgcaaggcatttgtgaaggtgctgcatggttgtcgtcagctcgtgccgtgaggtgtcggcttaagtgccataacgagcgcaacccttatcttcagttactaacaggtcatgctgaggactctggagagactgccgtcgtaagatgtgaggaaggtggggatgacgtcaaatcagcacggcccttacgtccggggctacacacgtgttacaatggggggtacagaaggccgctacctggtgacaggatgccaatcccaaaaacctctctcagttcggatcgaagtctgcaacccgacttcgtgaagctggattcgctagtaatcgcgcatcagccatggcgcggtgaatacgttcccgggccttgtacacaccgcccgtcaagccatgaaagccgggggtacctgaagtacgtaaccgcaaggagcgtcctagggtaaaactggtaattggggctaagtcgtaacaaggtagccgtaccggaaggtgcggytggatcacctcctt

SEQ ID NO: 136 - DNA, 1514 bp, synthetic construct
  misc_feature 1..1514: note = Synthetic: FBI00267
  source 1..1514: mol_type = other DNA
tggagagtttgatcctggctcaggacgaacgctggcggcgtgcttaacacatgcaagtcgaacgagaagcttatgattgattcttcggaagatttcataagcggaaagtggcgaacgggtgagtaacgcgtaggtaaccaacctcatacagggggatagcccagggaaacttggattaaacccgcataagaccacgctatcgcatgataaagtggtaaaaactccggtggtatgagacggacctgcgtcttattaggtagttggtgaggtaacggctcaccaagccgacgatgagtagccgacctgagagggtgatcggccacattgggactgagacacggcccagactcctacgggaggcagcagtggggaatattgcgcaatgggggcaaccctgacgcagcaacgccgcgtgagcgatgaaggttttcggatcgtaaagctctgttctgagggaagataatgacggtacctcaggagaaagccccggctaactacgtgccagcagccgcggtaatacgtagggggcaagcgttgtccggattcactgggcgtaaagagcacgtaggcggttaattaagtcaggtgtgaaagttttcggctcaaccggaaaagtgcacttgaaactggataacttgagtatcggagaggtaagcggaattcctagtgtagcggtgaaatgcgtagagattaggaagaacaccggtggcgaaggcggcttactggacgataactgacgctgaggtgcgaaagcgtggggagcgaacaggattagataccctggtagtccacgccgtaaacgatgaatactaggtgttggggtaactcagtgccgcagttaacacattaagtattccgcctggggagtacgctcgcaagagtgaaactcaaaggaattgacgggggcccgcacaagcagcggagcatgtggtttaattcgaagcaacgcgaagaaccttaccaggtcttgacatcctttgaccgtctaagagattagactttccttcgggacaaagagacaggtggtgcatggttgtcgtcagctcgtgtcgtgagatgttgggttaagtcccgcaacgagcgcaacccttgtgtttagttactaacattcagttgaggactctagacagactgcccttgaaagagggaggaaggtggggacgacgtcaaatcatcatgccccttacgacctgggctacacacgtgctacaatggtctgtacaaagggttgcgaagcagtgatgccaagctaatctcaaaaagcagatctcagttcggattgcaggctgcaactcgcctgcatgaagtcggagttgctagtaatcgcgaatcagaatgtcgcggtgaatgcgttcccgggccttgtacacaccgcccgtcacaccacgagagttggtaacacccgaagccagtgagctaaccgtaaggaggcagctgtcgaaggtgggatcagtaattggggtgaagtcgtaacaaggtagccgtatcggaaggtgcggytggatcacctcctt

SEQ ID NO: 137 - DNA, 1384 bp, synthetic construct
  misc_feature 1..1384: note = Synthetic: FBI00269
  source 1..1384: mol_type = other DNA
tcgaggggcagcataatggatagcaatatctatggtggcgaccggcgcacgggtgcgtaacgcgtatgcaacctacctttaacagggggataacactgagaaattggtactaataccccataatatcatagaaggcatcttttatggttgaaaattccgatggttagagatgggcatgcgttgtattagctagttggtggggtaacggctcaccaaggcgacgatacatagggggactgagaggttaaccccccacactggtactgagacacggaccagactcctacgggaggcagcagtgaggaatattggtcaatggacgcaagtctgaaccagccatgccgcgtgcaggatgacggctctatgagttgtaaactgcttttgtacgagggtaaacgcagatacgtgtatctgtctgaaagtatcgtacgaataaggatcggctaactccgtgccagcagccgcggtaatacggaggattcaagcgttatccggatttattgggtttaaagggtgcgtaggcggtttgataagttagaggtgaaatttcggggctcaaccctgaacgtgcctctaatactgttgagctagagagtagttgcggtaggcggaatgtatggtgtagcggtgaaatgcttagagatcatacagaacaccgattgcgaaggcagcttaccaaactatatctgacgttgaggcacgaaagcgtggggagcaaacaggattagataccctggtagtccacgcagtaaacgatgataactcgttgtcggcgatacacagtcggtgactaagcgaaagcgataagttatccacctggggagtacgttcgcaagaatgaaactcaaaggaattgacgggggcccgcacaagcggaggaacatgtggtttaattcgatgatacgcgaggaaccttacccgggcttgaaagttagcgacgattcttgaaagaggatttcccttcggggcgcgaaactaggtgctgcatggttgtcgtcagctcgtgccgtgaggtgtcgggttaagtcccataacgagcgcaacccctaccgttagttgccatcaggtgaagctgggcactctggcgggactgccggtgtaagccgagaggaaggtggggatgacgtcaaatcagcacggcccttacgtccggggctacacacgtgttacaatggtaggtacagagggcagctacccagcgatgggatgcgaatctcgaaagcctatctcagttcggattggaggctgaaacccgcctccatgaagttggattcgctagtaatcgcgcatcagccatggcgcggtgaatacgttcccgggccttgtacacaccgcccgtcaagccatgggagccgggggtgcctgaagttcgtgaccgcaagna

SEQ ID NO: 138 - DNA, 1277 bp, synthetic construct
  misc_feature 1..1277: note = Synthetic: FBI00270
  source 1..1277: mol_type = other DNA
tgggananccctggganctggggataatactggataggcaattattcctgtaatggttttttgtttaaatgttttttcgcctaaggatgggtctgcggccgattaggtagttggttaggtaatggcttaccaagcctttgatcggtacgggttgtgagagcaagagcccggagatggaacctgagacaaggttccaggccctacggggtgcagcaggcgcgaaacctccgcaatgtgagaaatcgcgacggggggatcccaagtgccattcttaacgggatggcttttcattagtgtaaagagcttttggaataagagctgggcaagaccggtgccagccgccgcggtaacaccggcagctctagtggtagcagtttttattgggcctaaagcgtccgtagccggtttaataagtctctggtgaaatcctgcagcttaactgtgggaattgctggagatactattagacttgagatcgggagaggttagaggtactcccagggtagaggtgaaattctgtaatcctgggaggaccgcctgttgcgaaggcgtctgactggaacgattctgacggtgagggacgaaagctaggggcgcgaaccggattagatacccgggtagtcctagctgtaaacgatgcggacttggtgttggggtggctttgagctgtcccagtgccgaagggaagctgttaagtccgccgcctgggaagtacggtcgcaagactgaaacttaaaggaattggcgggggagcaccacaacgcgtggagcctgcggtttaattggattcaacgccggacatctcaccagaggcgacagctgtatgatagccaggttgatgactttgcttgactagctgagaggaggtgcatggccgccgtcagctcgtaccgtgaggcgtcctgttaagtcaggcaacgagcgagacccacgctcttagttaccagcggatccttttttggatgccgggcacactaaggggaccgcctatgataaataggaggaaggagtggacgacggtaggtccgtatgccccgaatcctctgggcaacacgcgggctacaatggctgagacaatgggttccgacgccgaaaggcggaggtaatcctctaaacttagtcgtagttcggattgaggactgtaactcgttctcatgaagctggaatgcgtagtaatcgcgtgtcacaatcgcgcggtgaatacgtccctgctccttgcacacaccgcccgtcacgccacccaaaaagggattggatgaggatgtaatgttttgtna

SEQ ID NO: 139 - DNA, 1390 bp, synthetic construct
  misc_feature 1..1390: note = Synthetic: FBI00271
  source 1..1390: mol_type = other DNA
gcaagtcgaggggcagcattttagtttgcgttgcaaacgtaaagatggcgaccggcgcacgggtgagtaacacgtatccaacctgccgataactcggggatagcctttcgaaagaaagattaatatccgatagtatattaaaaccgcatggttttactattaaagaatttcggttatcgatggggatgcgttccattagtttgttggcggggtaacggcccaccaagactacgatggataggggttctgagaggaaggtcccccacattggaactgagacacggtccaaactcctacgggaggcagcagtgaggaatattggtcaatggacgagagtctgaaccagccaagtagcgtgaaggatgactgccctatgggttgtaaacttcttttatatgggaataaagtattccacgtgtggaattttgtatgtaccatatgaataaggatcggctaactccgtgccagcagccgcggtaatacggaggatccgagcgttatccggatttattgggtttaaagggagcgtaggtggattgttaagtcagttgtgaaagtttgcggctcaaccgtaaaattgcagttgaaactggcagtcttgagtacagtagaggtgggcggaattcgtggtgtagcggtgaaatgcttagatatcacgaagaactccgattgcgaaggcagctcactagactgcaactgacactgatgctcgaaagtgtgggtatcaaacaggattagataccctggtagtccacacagtaaacgatgaatactcgctgtttgcgatatacagtaagcggccaagcgaaagcattaagtattccacctggggagtacgccggcaacggtgaaactcaaaggaattgacgggggcccgcacaagcggaggaacatgtggtttaattcgatgatacgcgaggaccntacccgggcttaaattgcaatgaatatctggaaacaggttagccgcaaggcatttgtgaaggtgctgcatggtggtcgtcagttcgtcccgtgaggtgtcggcttaagtgccataacgagcgcaacctttttctttagttattaccaggtcatgctgaggattttagagagactcccttcgtaagatgtgaggaaggtggggatgacgtcaaatcagcacggcccttacgtccggggttacacacgtgttccaatggggggtacagaaggcagctacctggtgacaggatgctaatcccaaaaacctctctcagttcggatcgaagtctgcaacccgacttcgtgaagctggattcgctagtaatcgcgcatcagccatggcgcggtgaatacgttcccgggccttgtacacaccgcccgtcaagccatgaaagccgggggtacctgaagtacgtaaccgcaagga

SEQ ID NO: 140 - DNA, 1404 bp, synthetic construct
  misc_feature 1..1404: note = Synthetic: FBI00273
  source 1..1404: mol_type = other DNA
tgnnagtcgaggggcagcggggaggtagcaatacctttgccggcgaccggcgcacgggtgagtaacacgtatgcaatccacctgtaacagggggataacccggagaaatccggactaataccccataatatgggcgctccgcatggagggttcattaaagagagcaattttggttacngacgagcatgcgctccattagccagttggcggggtaacggcccaccaaagcgacgatggataggggttctgagaggaaggtcccccacattggaactgagacacggtccaaactcctacgggaggcagcagtgaggaatattggtcaatggtcggcagactgaaccagccaagtcgcgtgagggaagacggccctacgggttgtaaacctcttttgtcggagagtaaagtgcgctacgtgtagcgtattgcaagtatccgaagaaaaagcatcggctaactccgtgccagcagccgcggtaatacggaggatgcgagcgttatccggatttattgggtttaaagggtgcgtaggcggcacgccaagtcagcggtgaaatttccgggctcaacccggagtgtgccgttgaaactggcgagctagagtgcacaagaggcaggcggaatgcgtggtgtagcggtgaaatgcatagatatcacgcagaaccccgattgcgaaggcagcctgctagggtgaaacagacgctgaggcacgaaagcgtgggtatcgaacaggattagataccctggtagtccacgcagtaaacgatgaatactaactgtttgcgatacaatgtaagcggtacagcgaaagcgttaagtattccacctggggagtacgccggcaacggtgaaactcaaaggaattgacggggggcccgcacaagcggaggaacatgtggtttaattcgatgatacgcgaggaaccttacccgggctcaaacgcagggggaatgccggtgaaagccggcagctagcaatagtcacctgcgaggtgctgcatggttgtcgtcagctcgtgccgtgaggtgtcggcttaagtgccataacgagcgcaacccctatcgacagttactaacgggtgaagccgaggactctgtcgagactgccggcgcaagccgcgaggaaggtggggatgacgtcaaatcagcacggcccttacgtccggggcgacacacgtgttacaatggcaggtacagaaggcagccagtcagcaatgacgcgcgantcccgaaaacctgtctcagttcggattggagtctgcaacccgactccatgaagctggattcgctagtaatcgcgcatcagccatggcgcggtgaatacgttcccgggccttgtacacaccgcccgtcaagccatggaagccgggagtacctgaagcatgcaaccgcaaggagcgtacga

SEQ ID NO: 141 - DNA, 1405 bp, synthetic construct
  misc_feature 1..1405: note = Synthetic: FBI00274
  source 1..1405: mol_type = other DNA
tgcagtcgaacgaagcacctctcccgaagattgacacagcttgctgtagattgattcatttgaggtgactgagtggcggacgggtgagtaacgcgtgggtaacctgcctcatagagggggacaacagttggaaacgactgctaataccgcatagtaagaaaaattcgcatgttttcttcttgaaagatttatcgctatgagatggacccgcgtctgattagctagttggtaaggtaacggcttaccaaggcgacgatcagtagccggcttgagagagtgaacggccacattgggactgagacacggcccaaactcctacgggaggcagcagtggggaatattgcacaatgggggaaaccctgatgcagcgacgccgcgtgagtgaagaagtatttcggtatgtaaagctctatcagcagggaagataatgacggtacctgactaagaagccccggctaactacgtgccagcagccgcggtaatacgtagggggcaagcgttatccggatttactgggtgtaaagggtgcgtaggtggcaaggcaagtcagatgtgaaagcccggggctcaaccccggtactgcatttgaaactgtctagctagagtgcaggagaggtaagcggaattcctagtgtagcggtgaaatgcgtagatattaggaggaacaccagtggcgaaggcggcttactggactgtaactgacactgaggcacgaaagcgtggggagcaaacaggattagataccctggtagtccacgccgtaaacgatgaatactaggtgtcggggcccacagggcttcggtgccgcagcaaacgcattaagtattccacctggggagtacgttcgcaagaatgaaactcaaaggaattgacggggacccgcacaagcggtggagcatgtggtttaattcgaagcaacgcgaagaaccttaccaagtcttgacatccttctgaccgttccttagccggaacttnccttcggagcagaagtgacaggtggtgcatggttgtcgtcagctcgtgtcgtgagatgttgggttaagtcccgcaacgagcgcaacccttatccttagtagccagcggttcggccgggcactctggggagactgccggagataatccggaggaaggtggggatgacgtcaaatcatcatgccccttatgacttgggctacacacgtgctacaatggcggtaacaaagggaagcagcctcgcgagagtgagcaaaccccaaaaatgccgtctcagttcggattgtagtctgcaactcgactacatgaagctggaatcgctagtaatcgcagatcagaatgctgcggtgaatacgttcccgggtcttgtacacaccgcccgtcacaccatgggagtcggatatgcccgaagccagtgacccaacnnnaagga

SEQ ID NO: 142 - DNA, 1388 bp, synthetic construct
  misc_feature 1..1388: note = Synthetic: FBI00277
  source 1..1388: mol_type = other DNA
tgcagtcgaggggcatcacgaggtagcaatactttggtggcgaccggcgcacgggtgcgtaacgcgtatgtaacctacctataacaggggcataacactgagaaattggtactaattccccataatattcggagaggcatctctccgggttgaaaactccggtggttatagatggacatgcgttgtattagctagttggtgaggtaacggctcaccaaggcaacgatacatagggggactgagaggttaaccccccacactggtactgagacacggaccagactcctacgggaggcagcagtgaggaatattggtcaatggacgcaagtctgaaccagccatgccgcgtgcaggaagacggctctatgagttgtaaactgcttttgtacgagggtaaactcacctacgcgtaggtgactgaaagtatcgtacgaataaggatcggctaactccgtgccagcagccgcggtaatacggaggattcaagcgttatccggatttattgggtttaaagggtgcgtaggcggtttgataagttagaggtgaaatcccggggcttaactccggaactgcctctaatactgttagactagagagtagttgcggtaggcggaatgtatggtgtagcggtgaaatgcttagagatcatacagaacaccgattgcgaaggcagcttaccaaactatatctgacgttgaggcacgaaagcgtggggagcaaacaggattagataccctggtagtccacgcagtaaacgatgataactcgttgtcggcgatacacagtcggtgactaagcgaaagcgataagttatccacctggggagtacgttcgcaagaatgaaactcaaaggaattgacgggggcccgcacaagcggaggaacatgtggtttaattcgatgatacgcgaggaaccttacccgggcttgaaagttactgacgattctggaaacaggatttcccttcggggcaggaaactaggtgctgcatggttgtcgtcagctcgtgccgtgaggtgtcgggttaagtcccataacgagcgcaacccctaccgttagttgccatcaggtcaagctgggcactctggcgggactgccggtgtaagccgagaggaaggtggggatgacgtcaaatcagcacggcccttacgtccggggctacacacgtgttacaatggtaggtacagagggtcgctactccgtgaggagatgccaatctcgaaagcctatctcagttcggattggaggctgaaacccgcctccatgaagttggattcgctagtaatcgcgcatcagccatggcgcggtgaatacgttcccgggccttgtacacaccgcccgtcaagccatggaagctgggggtgcctgaagttcgtgaccgcaangagc

SEQ ID NO: 143 - DNA, 1398 bp, synthetic construct
  misc_feature 1..1398: note = Synthetic: FBI00278
  source 1..1398: mol_type = other DNA
tgcagtcgancgaagcaccnttgngacagannnnttcgganggacagactntggtgacttagtggcggacgggtgagtaacgcgtgggtaacctgccctgtactgggggacaacagttggaaacgactgctaataccgcataagcgcacagcatcgcatgatgcagtgtgaaaaactccggtggtacaggatggacccgcgtctgattagctagttggtgaggtaacggcttaccaaggcaacgatcagtagccgacttgagagagtgatcggccacattgggactgagacacggcccaaactcctacgggaggcagcagtggggaatattgcacaatgggggaaaccctgatgcagcgacgccgcgtgaaggaagaagtatttcggtatgtaaacttctatcagcaaggaagaaaatgacggtacttgactaagaagccccggctaaatacgtgccagcagccgcggtaatacgtatggggcaagcgttatccggatttactgggtgtaaagggagcgtaggcggtaagacaagtcagaagtgaaaggctggggctcaaccctgggactgcttttgaaactgtctaactagagtgcaggagaggtaagtggaattcctagtgtagcggtgaaatgcgtagatattaggaggaacaccagtggcgaaggcggcttactggactgtaactgacgctgaggctcgaaagcgtggggagcaaacaggattagataccctggtagtccacgccgtaaacgatgaatactaggtgttgggggaacaatagttcttcggtgccgcagcaaacgcaataagtattccacctggggagtacgttcgcaagaatgaaactcaaaggaattgacggggacccgcacaagcggtggagcatgtggtttaattcgaagcaacgcgaagaaccttacctgctcttgacatcccactgaccggtcagtaatgtgaccttttcttcggaacagtggagacaggtggtgcatggttgtcgtcagctcgtgtcgtgagatgttgggttaagtcccgcaacgagcgcaacccttatctttagtagccagcatttaagatgggcactctagagagactgccagggataacctggaggaaggtggggatgacgtcaaatcatcatgccccttatgagcagggctacacacgtgctacaatggcgtaaacaaagggaagcgagaccgtgaggtggagccaatcccaaaaataacgtctcagttcggattgtagtctgcaactcgactacatgaagctggaatcgctagtaatcgcgaatcagaatgtcgcggtgaatacgttcccgggtcttgtacacaccgcccgtcacaccatgggagttggatatgcccgaagtcagtgacccaaccgtaaggagg

SEQ ID NO: 144 - DNA, 1379 bp, synthetic construct
  misc_feature 1..1379: note = Synthetic: FBI00281
  source 1..1379: mol_type = other DNA
tgcagtcgaacgatgaaaccnancctcgggcggacatgaagtggcgaacgggtgagtaacacgtgaccgacctgccccccgcccagggacagccccccgaaagggggattaatacctggtactccggaagcggcgcatgccgcggccgggaaagctccggcggcgggggatggggtcgcggcccatcaggtagacggcggggcagaggcccaccgtgccgacgacgggtagccgggttgagagaccgaccggccacattgggactgagatacggcccagactcctacgggaggcagcagtggggaattttgcgcaatgggggcaaccctgacgcagcaacgccgcgtgcgggacgaaggcgtccgcgtcgtaaaccgctttcagcggggaacacttgaacgagggtacccgcagaagaagccccggctaaatacgtgccagcagccgcggtaatacgtatggggcgagcgttatccggattcattgggcgtaaagcgcgcgtaggcggagcgctaagcgggacctctaacccgagggctcaacccccggccgggtcccgaactggcgctctcgagtgcggtaggggagagcggaattcccggtgtagcggtggaatgcgcagatatcgggaagaacaccgacggcgaaggcagctctctgggccgaaactgacgctgaggcgcgaaagctgggggagcgaacaggattagataccctggtagtcccagccgtaaacgatgggcgctaggtgtgggggagcagatcccccgtgccgcagccaacgcattaagcgccccgcctggggagtacggccgcaaggctaaaactcaaaggaattgacgggggcccgcacaagcagcggagcatgtggcttaattcgaagcaacgcgaagaaccttaccagggcttgacatgcaggtgaagcggcggagacgccgtggccgagaggagcctgcacaggtggtgcatggctgtcgtcagctcgtgtcgtgagatgttgggttaagtcccgcaacgagcgcaacccctgccccgtgttgccagcattcagttggggactcgcgggggactgccggcgtcaagccggaggaaggtggggacgacgtcaagtcatcatgccccttatgccctgggccgcacacgtgctacaatggccggtacagagggttgcgaccccgcgagggggagcggatcccgcaaagccggccccagttcggatcggaggctgcaacccgcctccgtgaagccggagttgctagtaatcgcggatcagcacgccgcggtgaatacgttcccgggccttgtacacaccgcccgtcacaccacccgagtcgtctgcacccgaagccgccnnnnnnaacccccctgggggcggaggc

SEQ ID NO: 145 - DNA, 1398 bp, synthetic construct
  misc_feature 1..1398: note = Synthetic: FBI00288
  source 1..1398: mol_type = other DNA
tgcagtcgaacgaagcgatggagaacggagatttcggttgaagttttctattgactgagtggcggacgggtgagtaacgcgtgggtaacctgccctatacagggggataacagttagaaatgactgctaataccgcataagcgcacagcttcgcatgaagcggtgtgaaaaactgaggtggtataggatggacccgcgttggattagctagttggtgaggtaacggcccaccaaggcgacgatccatagccggcctgagagggtgaacggccacattgggactgagacacggcccaaactcctacgggaggcagcagtggggaatattgcacaatgggggaaaccctgatgcagcgacgccgcgtgaaggaagaagtatctcggtatgtaaacttctatcagcagggaagaaagtgacggtacctgactaagaagccccggctaattacgtgccagcagccgcggtaatacgtaaggggcaagcgttatccggatttactgggtgtaaagggagcgtagacggtttggcaagtctgatgtgaaaggcatgggctcaacctgtggactgcattggaaactgtcagacttgagtgccggagaggcaagcggaattcctagtgtagcggtgaaatgcgtagatattaggaggaacaccagtggcgaaggcggcctgctggacggtaactgacgttgaggctcgaaagcgtggggagcaaacaggattagataccctggtagtccacgctgtaaacgatgaatactaggtgtcgggtggcaaagccattcggtgccgcagcaaacgcaataagtattccacctggggagtacgttcgcaagaatgaaactcaaaggaattgacggggacccgcacaagcggtggagcatgtggtttaattcgaagcaacgcgaagaaccttaccaaatcttgacatccctctgaccgggaagtaatgttcccttttcttcggaacagaggagacaggtggtgcatggttgtcgtcagctcgtgtcgtgagatgttgggttaagtcccgcaacgagcgcaacccttattcttagtagccagcaggtaaagctgggcactctagggagactgccagggataacctggaggaaggtggggatgacgtcaaatcatcatgccccttatgatttgggctacacacgtgctacaatggcgtaaacaaagggaagcgaaggggtgacctggagcaaatctcaaaaataacgtctcagttcggattgtagtctgcaactcgactacatgaagctggaatcgctagtaatcgcgaatcagaatgtcgcggtgaatacgttcccgggtcttgtacacaccgcccgtcacaccatgggagtcagtaacgcccgaagtcagtgacccaaccgaaagganggagc

SEQ ID NO: 146 - DNA, 1388 bp, synthetic construct
  misc_feature 1..1388: note = Synthetic: FBI00289
  source 1..1388: mol_type = other DNA
aacggcagcatgggggcaaccctgatggcgagtggcgaacgggtgagtaatatatcggaacgtacccaagagtgggggacaaccagtcgaaagattggctaataccgcatacgatctaaggatgaaagtgggggaccttcgggcctcacgctcctggagcggccgatagctgattagctagttggtggggtaaaggcctaccaaggcgatgatcagtagctggtctgagaggacgatcagccacactgggactgagacacggcccagactcctacgggaggcagcagtggggaattttggacaatgggggaaaccctgatccagcaatgccgcgtgagtgaagaaggccttcgggttgtaaagctcttttgtcagggaagaaattgattgggctaatatcccgattagatgacggtacctgaagaataagcaccggctaactacgtgccagcagccgcggtaatacgtagggtgcgagcgttaatcggaattactgggcgtaaagcgtgcgcaggcggttgtgtaagacagatgtgaaatgcccgggctcaacctgggaattgcatttgtgactgcacggctagagtgtgtcagaggggggtagaattccacgtgtagcagtgaaatgcgtagatatgtggaggaataccgatggcgaaggcagccccctgggataacactgacgctcatgcacgaaagcgtggggagcaaacaggattagataccctggtagtccacgccctaaacgatgtcgactagttgttgggtcttaattgacttagtaacgcagctaacgcgtgaagtcgaccgcctggggagtacggtcgcaagattaaaactcaaaggaattgacggggacccgcacaagcggtggatgatgtggattaattcgatgcaacgcgaaaaaccttacctacccttgacatgtcaggaatcccgaagagatttgggagtgctcgaaagagagcctgaacacaggtgctgcatggctgtcgtcagctcgtgtcgtgagatgttgggttaagtcccgcaacgagcgcaacccttgtcattagttgctacgaaagggcactctaatgaaactgccggtgacaaaccggaggaaggtggggatgacgtcaagtcctcatggcccttatgggtagggcttcacacgtcatacaatggtgcatacagagggttgccaacccgagagggggagctaatctcagaaagtgcatcgtagtccggattgaagtctgcaactcgacttcatgaagttggaatcgctagtaatcgcggatcagcatgtcgcggtgaatacgttcccgggtcttgtacacaccgcccgtcacaccatgggagcgggttttaccagaagtaggtagcttaaccgtaaggagggcgct

SEQ ID NO: 147 - DNA, 1381 bp, synthetic construct
  misc_feature 1..1381: note = Synthetic: FBI00290
  source 1..1381: mol_type = other DNA
ttannnntgcagtcgaacgaagacnntnctngagtgcttgcacttggannggactgagtggcggacgggtgagtaacgcgtgggtaacctgccttatacagggggataacagttggaaacgactgctaataccgcataagcgcacggtatcgcatgatacagtgtgaaaaactccggtggtataagatggacccgcgtctgattagcttgttggtgaggtaacggcccaccaaggcgacgatcagtagccggcctgagagggtgaacggccacattgggactgagacacggcccaaactcctacgggaggcagcagtggggaatattgcacaatgggggaaaccctgatgcagcgacgccgcgtgagtgatgaagtatttcggtatgtaaaactctatcagcagggaagataatgacggtacctgactaagaagcaccggctaaatacgtgccagcagccgcggtaatacgtatggtgcaagcgttatccggatttactgggtgtaaagggtgcgtaggtggtatggcaagtcagaagtgaaaggctggggctcaacccccgggactgcttttgaaactgtcaaactagagtacaggagaggaaaagcggaattcctagtgtagcggtgaaatgcgtagatattaggaggaacaccagtggcgaaggcggctttctggactgaaactgacactgaggcacgaaagcgtggggagcaaacaggattagataccctggtagtccacgccgtaaacgatgaatactaggtgtcgggcagcatagctgttcggtgccgcagcaaacgcattaagtattccacctggggagtacgttcgcaagaatgaaactcaaaggaattgacggggacccgcacaagcggtggagcatgtggtttaattcgaagcaacgcgaagaaccttaccaggtcttgacatcccgatgaccgcttcttaatcgaaacttttcttcggaacatcggtgacaggtggtgcatggttgtcgtcagctcgtgtcgtgagatgttgggttaagtcccgcaacgagcgcaacccctattttcagtagccagcattttggatgggcactctggagagactgcccgggataaccgggaggaaggtggggatgacgtcaaatcatcatgccccttatgatctgggctacacacgtgctacaatggctgttacagagagaagcgaatccgcgaggaagagcaaacctcaaaaaggcagtctcagttcggattgtagtctgcaactcgactacatgaagctggaatcgctagtaatcgcgaatcagaatgtcgcggtgaatacgttcccgggtcttgtacacaccgcccgtcacaccatgggagttggaaatgcccgaagtcagtgaccc

SEQ ID NO: 148 - DNA, 1262 bp, synthetic construct
  misc_feature 1..1262: note = Synthetic: FBI00292
  source 1..1262: mol_type = other DNA
ggnnngggatnnccctgggaactggggataatactggataggcaattattcctgtaatggttttttgtttaaatgttttttcgcctaaggatgggtctgcggccgattaggtagttggttaggtaatggcttaccaagcctttgatcggtacgggttgtgagagcaagagcccggagatggaacctgagacaaggttccaggccctacggggtgcagcaggcgcgaaacctccgcaatgtgagaaatcgcgacggggggatcccaagtgccattcttaacgggatggcttttcattagtgtaaagagcttttggaataagagctgggcaagaccggtgccagccgccgcggtaacaccggcagctctagtggtagcagtttttattgggcctaaagcgtccgtagccggtttaataagtctctggtgaaatcctgcagcttaactgtgggaattgctggagatactattagacttgagatcgggagaggttagaggtactcccagggtagaggtgaaattctgtaatcctgggaggaccgcctgttgcgaaggcgtctgactggaacgattctgacggtgagggacgaaagctaggggcgcgaaccggattagatacccgggtagtcctagctgtaaacgatgcggacttggtgttggggtggctttgagctgtcccagtgccgaagggaagctgttaagtccgccgcctgggaagtacggtcgcaagactgaaacttaaaggaattggcgggggagcaccacaacgcgtggagcctgcggtttaattggattcaacgccggacatctcaccagaggcgacagctgtatgatagccaggttgatgactttgcttgactagctgagaggaggtgcatggccgccgtcagctcgtaccgtgaggcgtcctgttaagtcaggcaacgagcgagacccacgcccttagttaccagcggatccttttggatgccgggcacactaaggggaccgcctatgataaataggaggaaggagtggacgacggtaggtccgtatgccccgaatcctctgggcaacacgcgggctacaatggctgagacaatgggttccgacgccgaaaggcggaggtaatcctctaaacttagtcgtagttcggattgaggactgtaactcgttctcatgaagctggaatgcgtagtaatcgcgtgtcacaatcgcgcggtgaatacgtccctgctccttgcacacaccgcccgtcacgccacccaaaaagggattgngatga